(19) United States
(12) Patent Application Publication
(10) Pub. No.: US 2010/0184033 A1
(43) Pub. Date: Jul. 22, 2010

(54) METHODS TO ACCELERATE THE ISOLATION OF NOVEL CELL STRAINS FROM PLURIPOTENT STEM CELLS AND CELLS OBTAINED THEREBY

(76) Inventors: Michael D. West, Mill Valley, CA (US); Geoffrey Sargent, San Lorenzo, CA (US); James T. Murai, San Bruno, CA (US); Steven Kessler, Belmont, CA (US); Karen Chapman, Mill Valley, CA (US); David Larocca, Encinitas, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 12/504,630

(22) Filed: Jul. 16, 2009

Related U.S. Application Data
(60) Provisional application No. 61/081,325, filed on Jul. 16, 2008.
(60) Provisional application No. 61/178,457, filed on May 14, 2009.

(51) Int. Cl. C12Q 1/68; C12N 5/07; C12N 5/071

(52) U.S. Cl. 435/6; 435/325; 435/366

(57) ABSTRACT

Aspects of the present invention relate to methods to differentiate pluripotent primordial stem cells, such as human embryonic stem (“hES”) cells, human embryonic germ (“hEG”) cells, human embryo-derived (“hED”) cells and human embryonal carcinoma (“hEC”) cells, to obtain subpopulations of cells from heterogeneous mixtures of cells, wherein the subpopulation of cells possess reduced differentiation potential compared to the original pluripotent stem cells and where the subpopulation is capable of being propagated 20 or more population doublings. This invention also provides novel compositions of such subpopulations of cells and methods to propagate and differentiate said cells.

### CROSS REFERENCE

This application claims the benefit under 35 U.S.C. §119(e) of the following provisional patent applications: Application Ser. No. 61/081,325, entitled “METHODS AND REAGENTS FOR THE IDENTIFICATION, ISOLATION AND PROPAGATION OF EMBRYONIC PROGENITOR CELL LINES” filed Jul. 16, 2008, and Application Ser. No. 61/178,457, entitled “METHODS TO ACCELERATE THE ISOLATION OF NOVEL CELL STRAINS FROM PLURIPOTENT STEM CELLS AND CELLS OBTAINED THEREBY” filed May 14, 2009. The entirety of both applications is incorporated herein by reference.

### TABLES PROVIDED IN ELECTRONIC FORM

This application includes Table XXI, Table XXII, Table XXIII, and Table XXIV. Table XXI is eight text files named “BIOT-013_Table_XXIA” 44 KB in size created on Jul. 16, 2009, “BIOT-013_Table XXIB” 115 KB in size created on Jul. 16, 2009, “BIOT-013_Table_XXIC” 104 KB in size created on Jul. 16, 2009, “BIOT-013_Table_XXID” 134 KB in size created on Jul. 16, 2009, “BIOT-013_Table_XXIE” 78 KB in size created on Jul. 16, 2009, “BIOT-013_Table_XXIF” 70 KB in size created on Jul. 16, 2009, “BIOT-013_Table_XXIG” 100 KB in size created on Jul. 16, 2009 and “BIOT-013_Table_XXIH” 39 KB in size created on Jul. 16, 2009. Table XXII is two text files named “BIOT-013_Table_XXIIA” 26 KB in size created on Jul. 16, 2009 and “BIOT-013_TableXXIIB” 12 KB in size created on Jul. 16, 2009. Table XXIII is eight text files named “BIOT-013_Table_XXIIIA” 121 KB in size created on Jul. 16, 2009, “1310T-013_Table_XXIIIB” 86 KB in size created on Jul. 16, 2009, “BIOT-013_Table_XXIIIC” 23 KB in size created on Jul. 16, 2009, “BIOT-013_Table_XXIIID” 135 KB in size created on Jul. 16, 2009, “BIOT-013_Table_XXIIIE” 61 KB in size created on Jul. 16, 2009, “BIOT-013_Table_XXIIIF” 42 KB in size created on Jul. 16, 2009, “BIOT-013_Table_XXIIIG” 64 KB in size created on Jul. 16, 2009 and “BIOT-013_Table_XXIIIH” 57 KB in size created on Jul. 16, 2009. Table XXIV is two text files named “BIOT-013_Table_XXIVA” 44 KB in size created on Jul. 16, 2009 and “BIOT-013_Table_XXIVB” 51 KB in size created on Jul. 16, 2009. The information contained in Tables XXI, XXII, XXIII and XXIV is hereby incorporated by reference in this application.

### FIELD OF THE INVENTION

This invention generally relates to methods to accelerate the isolation of novel cell strains from pluripotent stem cells and cells obtained by such methods. Specifically, this invention relates to methods to differentiate pluripotent primordial stem cells, such as human embryonic stem (“hES”) cells, human embryonic germ (“hEG”) cells, human embryo-derived (“hED”) cells and human embryonal carcinoma (“hEC”) cells, to obtain subpopulations of cells from heterogeneous mixtures of cells, wherein the subpopulation of cells possess reduced differentiation potential compared to the original pluripotent stem cells and where the subpopulation is capable of being propagated 20 or more population doublings. This invention also provides novel compositions of such subpopulations of cells and methods to propagate and differentiate said cells. More particularly, the invention relates to a two-step method wherein said pluripotent stem cells are first exposed to conditions that induce a heterogeneity of differentiation potential in said stem cells, and next a plating/propagation step allowing single cells or an oligoclonal cluster of similar cells with reduced breadth of differentiation potential than the original stem cells and that resulted from the original stem cells to expand in number while exposed to a combination of culture environments that determine conditions that promote propagation from one or a small cluster of cells. Said single cell or oligoclonal cell-derived populations of cells with a more restricted breadth of differentiation potential and cells capable of proliferation from the second step are characterized and formulated for use in research and therapy, and for the production of bioactive materials such as cell extracts, conditioned medium and extracellular matrix.

### BACKGROUND OF THE INVENTION

Advances in stem cell technology, such as the isolation and propagation in vitro of embryonic stem cells (“ES” cells including human ES cells (“hES” cells)) and related totipotent primordial stem cells including but not limited to, EG, EC, or ED cells (including human EG, EC, or ED cells), constitute an important new area of medical research. hES cells have a demonstrated potential to be propagated in the undifferentiated state and then to be induced subsequently to differentiate into any and all of the cell types in the human body, including complex tissues. In addition, many of these primordial stem cells are naturally telomerase positive in the undifferentiated state, thereby allowing the cells to be expanded extensively and subsequently genetically modified and clonally expanded. Since the telomere length of many of these cells is germ-line in length (approximately 15 kbp TRF length), differentiated cells derived from these immortal lines will naturally repress the expression of the catalytic component of telomerase (hTERT) and thereby become mortal, though the long initial telomere length allows for cells with long replicative capacity compared to fetal or adult-derived tissue. This has led to the suggestion that many diseases resulting from the dysfunction of cells may be amenable to treatment by the administration of hES-derived cells of various differentiated types (Thomson et al., Science 282:1145-1147 (1998)). Nuclear transfer studies have demonstrated that it is possible to transform a somatic differentiated cell back to a totipotent state such as that of embryonic stem (“ES”) cells (Cibelli et al., Nature Biotech 16:642-646 (1998)) or embryo-derived (“ED”) cells. The development of technologies to reprogram somatic cells back to a totipotent ES cell state, such as by the transfer of the genome of the somatic cell to an enucleated oocyte and the subsequent culture of the reconstructed embryo to yield ES cells, often referred to as somatic cell nuclear transfer (“SCNT”), offers a method to transplant ES-derived somatic cells with a nuclear genotype of the patient (Lanza et al., Nature Medicine 5:975-977 (1999)).

In addition to SCNT, other techniques exist to address the problem of transplant rejection, including the use of gynogenesis and androgenesis (see U.S. application Nos. 60/161,987, filed Oct. 28, 1999; Ser. Nos. 09/697,297, filed Oct. 27, 2000; 09/995,659, filed Nov. 29, 2001; 10/374,512, filed Feb. 27, 2003; PCT application no. PCT/US00/29551, filed Oct. 27, 2000; the disclosures of which are incorporated by reference in their entirety). In the case of a type of gynogenesis designated parthenogenesis, pluripotent stem cells may be manufactured without antigens foreign to the gamete donor and therefore useful in manufacturing cells that can be transplanted without rejection. In addition, parthenogenic stem cell lines can be assembled into a bank of cell lines homozygous or hemizygous in the HLA region to reduce the complexity of a stem cell bank in regard to HLA haplotypes.

Nevertheless, there remains a need for providing a means to direct the differentiation of totipotent or pluripotent stem cells into the many desired cell lineages present in the developing and developed mammalian body, under conditions which are compatible in either a general laboratory setting or in a good manufacturing processes (“GMP”) cell manufacturing facility where there is adequate documentation as to the purity and genetic normality of the cells.

Furthermore, there still remains a need to describe methods to identify cells derived from such pluripotent stem cells that are capable of being propagated in vitro, methods to identify culture conditions for propagating cells derived from pluripotent stem cells, precise definition relating to the materials that have come into physical contact with the cells, precise definition of the presence or absence of pathogens in such cells, and evidence as to whether any undifferentiated or other cell types, such as fibroblastic cells, contaminate the cell formulation derived from such cells planned for therapeutic use, and methods to identify such purified populations of cells that are capable of expansion in number in a target tissue and/or stable engraftment. Also, there is a need to derive cells from pluripotent stem cells, such derived cells being more differentiated than the parent pluripotent stem cells but still being progenitor cells that can differentiate further.

Furthermore, while there are numerous publications relating to the differential expression of genes, including but not limited to, differentiation-related genes such as homeobox-containing genes, in mouse and avian species, such data do not necessarily apply to other species such as hES-derived cells, and such published results often result from histological studies of limited tissues and whole tissues where it is not possible to determine precisely what cell types differentially express particular genes in the course of development. As a result, there is a need to determine what genes and combinations of genes provide useful markers of defined and clonal differentiation pathways in various species including avian species and mammalian species such as human. Such markers would allow the correct identification of cells derived from pluripotent stem cells such as hES cells. Furthermore, a database of collated gene expression patterns of numerous cell types differentiated from pluripotent stem cells such as hES cells allows the use of clustering algorithms to identify a novel cell type by displaying to what cell type in the existing database it is similar or essentially identical. Currently, numerous studies of hES-derived cells are problematic in that they are making poorly justified assumptions regarding the pattern of gene expression in early human development. Such a database is thus needed.

One of the major recurrent problems with culturing mammalian differentiated cell types in vitro is the preservation of a pure culture of the differentiated cell type without having the culture overgrown with fibroblastic or other contaminating cell types. See, Ian Freshney, Culture of Animal Cells: A Manual of Basic Technique (5th Ed.), New York: Wiley Publishing, 2005, p. 217. Because heterogeneous cultures of immortal organisms, such as bacteria or yeast cells, could be made homogeneous through means to isolate a population of cells from a single parent cell, efforts have been made to isolate populations of human and other mammalian cells of various types from a single parent cell (clonogenic growth). However, the traditional microbiological approach to the problem of culture heterogeneity, by isolating pure cell strains using cloning, has limited success in most primary cultures from fetal or adult tissue because of the poor cloning efficiencies. However, the cloning of primary cultures has been shown to be successful for certain cell types, for example, for Sertoli cells (Zwain et al., Mol Cell Endocrinol., 80(1-3):115-26 (1991)), juxtaglomerular (Muirhead et al., Methods Enzymol., 191:152-67 (1990)) and glomerular (Troyer & Kreisberg, Methods Enzymol., 191:141-52 (1990)) cells from kidney, oval cells from liver (Suh et al., Tissue Eng., 9(3):411-20 (2003)), and satellite cells from skeletal muscle (Zeng et al., Poult Sci., 81(8):1191-8 (2002); McFarland et al., Comp Biochem Physiol C Toxicol Pharmacol., 134(3):341-51 (2003); Hashimoto et al., Development, 131(21):5481-90 (2004)) and separation of different lineages from adult stem cell populations has been reported (Young et al., Anat Rec A Discov Mol Cell Evol Biol., 276(1):75-102 (2004)). Therefore, while the generation of clonogenic populations of cells has demonstrated its usefulness in generating a limited number of differentiated cell types free of contaminating cells, there still remains a need to describe methods for propagating cell types and culture systems, such as the early embryonic cell lineages derived from hES, hEG, hiPS, hEC or hED cells.

In addition, a further problem with culturing human cells is the inability to expand the number of cells in the cell cultures to generate enough cells to be of practical and therapeutic applicability. This stems from the observation that most human cell clones from fetal or adult tissue sources senesce relatively early, such as when still replicating in the original colony or shortly thereafter (i.e. can only survive for a limited number of generations, thereby limiting many applications such as scale-up in the manufacturing process) (see, e.g., Smith et al., Proc. Natl. Acad. Sci., USA, v. 75(3), pp. 1253-1356 (1978)).

In addition, most cells derived from fetal or adult sources are not capable of being propagated at low densities, such as when deriving cultures from a single parent cell or from a small number of similar cells (oligoclonal). At low densities, the cells do not receive sufficient mitogenic signals to allow for extensive propagation. Therefore, even if the cells had sufficient replicative lifespan to generate a useful culture of cells, the cultivation of many somatic cells at low density is nevertheless nonpermissive for growth. For uncharacterized cell types such as hES-derived cell lines, there is no way of knowing which, if any, hES-derived cells are capable of propagation clonally or oligoclonally in vitro. In some cases, growth of some cell types can nevertheless be achieved at clonal densities by culturing the cells under specific conditions, such as in low ambient oxygen, on mitotically inactivated feeder cells, or with the addition of conditioned medium. However, such techniques have only been reported useful in generating stable cell lines for a few cell types, and success for any novel cell type is still highly uncertain.

While methods have been described to accomplish genetic selection, by the introduction of transgenes into pluripotent stem cells, wherein the expression of said transgene is dependent upon a differentiation-specific promoter sequence and said transgene imparts an ability to select a particular differentiated cell type from a mixture of heterogeneous cells (see, e.g., U.S. Pat. Nos. 5,733,727 and 6,015,671), such genetic selection techniques do not in themselves necessarily lead to purified populations of cells capable of being propagated in vitro nor do they provide the methods to accomplish such propagation. In addition, novel methods that do not result in genetically modified cells would be useful in simplifying the development of cell-based therapies.

Furthermore, patterns for the expression of various growth factors, receptors, and extracellular matrix components in the developing animal have been described. For example, Ford-Perriss et al., Clinical & Experimental Pharm. & Physiol. 28:493-503 (2001) describe the expression of growth factors such as members of the FGF family of growth factors in the developing mammalian CNS, yet the role of these and many other factors in the differentiation of pluripotent stem cells in vitro, or in the cultivation of cells derived from a single cell or a small number of cells committed to a common cell fate that were themselves differentiated from or are in the process of differentiating from pluripotent stem cells has not been described.

Finally, while there are descriptions of numerous cell types obtained from pluripotent stem cells such as human embryonic stem cells, there has been no description of a method to obtain cells from hES, hEG, hiPS, hEC or hED cells, wherein said cells display a prenatal gene expression phenotype consistent with cells and tissues of animals in their embryonic stage of development, which are normally progressively lost in further fetal development and in the subsequent adult animal. While animals, models, and molecular studies have revealed that there are different gene expression patterns in fetal vs. adult tissues, prior attempts via gene therapy to alter the pattern of gene expression in cells to more closely mimic that of the early prenatal state have not resulted in satisfactory results. Therefore, there remains a need to describe a means for identifying and propagating such cells from pluripotent stem cells. The identification of the prenatal patterns of gene expression in such cells will provide useful markers for subsequent identification of these cells that may be capable of regenerating tissue, i.e., capable of stromal/epithelial interactions that can be organize tissue, including but not limited to, innervation (such as neural axon outgrowth) and vascularization.

In summary, while numerous techniques to increase the frequency of a desired cell type in a complex mixture of cell types differentiated from pluripotent stem cells have been reported, there remains a problem of the preservation of the culture of a particular cell type, in particular, properties useful in facilitating the transplantation of such cells into organs and tissues including, but not limited to, properties unique to embryonic cells and tissues. In addition, there remains a need to identify novel means of generating uniform populations of cells with limited or even unitary differentiation potential from pluripotent stem cells such as hES cells, means to identify said cells capable of being propagated in vitro, and methods of generating and propagating such a culture.

### SUMMARY OF THE INVENTION

This invention solves the problems described above. This invention generally relates to methods to differentiate pluripotent stem cells, such as human embryonic stem cells (“hES”), human embryonic germ (“hEG”) cells, human embryonal carcinoma (“hEC”) cells and human embryo-derived (“hED”) cells, to obtain subpopulations of cells from heterogeneous mixtures of cells, wherein the subpopulation of cells possess reduced differentiation potential compared to the original pluripotent stem cells and where the subpopulation is capable of being propagated. This invention also provides novel compositions of such subpopulations of cells and methods to propagate such cells.

More particularly, the invention relates to a two-step method wherein pluripotent stem cells are first exposed to conditions that induce a heterogeneity of differentiation potential in said stem cells, and next a plating/propagation step allowing single cells or an oligoclonal cluster of similar cells with reduced differentiation potential than the original stem cells and that resulted from the original stem cells to expand in number while exposed to a combination of culture environments. Said single cell-derived populations of cells with a more restricted breadth of differentiation potential and cells capable of proliferation from the second step are characterized and formulated for use in research and therapy, and for the production of cell extracts, conditioned medium, and extracellular matrix of said cells for formulation and use for research and therapy.

This invention provides a method for deriving desired cell types (“derived cells”) from pluripotent stem cells such as hES, hEG, hiPS, hEC or hED cells (parent population). The derived cells possess reduced differentiation potential when compared to the pluripotent stem cells from which they were derived (parent pluripotent stem cell population). The derived cells comprise cells that have the ability to differentiate further, i.e., they are not terminally differentiated cells. In certain embodiments, the method of this invention comprises the steps of:

(1)(a) selecting all or a subset of differentiation conditions that may result in the differentiation of said parent pluripotent stem cells into a heterogeneous population of cells, wherein a plurality of said cells may be more differentiated than said parent pluripotent stem cells; (1)(b) exposing said parent pluripotent stem cells to said all or a subset of differentiation conditions from step (1)(a) for various time periods resulting in a heterogeneous population of cells comprising cells with reduced differentiation potential than said parent pluripotent stem cells, wherein a plurality of said cells may have reduced differentiation potential than said parent pluripotent stem cells;
(2)(a) culturing said heterogeneous population of cells from step (1)(b) in culture conditions wherein said single cells proliferate and the single cells and/or their progeny may be isolated as a clonal or oligoclonal culture of cells; wherein said heterogeneous population of cells may optionally be disaggregated to single cells prior to culturing, and (2)(b) propagating said clonal population of cells of step (2)(a), resulting in said derived cells, wherein said cells are more uniform in differentiation potential and have reduced differentiation potential compared to the parent pluripotent stem cell population. In certain embodiments, the cells in steps (2)(a) and (2)(b) are grown in the same medium, including the differentiation conditions, as the medium used in step (1)(b) to differentiate the parent pluripotent stem cells. Using the same, or substantially the same medium and growth factors has the advantage that the cells capable of proliferating clonally or oligoclonally are expanded in step (1)(b), increasing the number of propagating clones in steps (2)(a) and (2)(b). The resulting cells are “derived cells.” In certain embodiments of this method, the heterogeneous population of cells from step (1)(b) are obtained by allowing said parent pluripotent stem cells to differentiate for various periods of time without disaggregation, i.e., for the cells to incubate in the differentiation conditions for various time periods before optionally disaggregating them. In a further embodiment of this method, the heterogeneous population of cells from step (1)(b) are obtained by allowing said parent pluripotent stem cells to differentiate for various periods of time without disaggregation, and further, comprising the step of producing embryoid bodies using a variety of culture conditions for various time periods. In further embodiments of this method, the embryoid bodies are differentiated for various time periods. In certain embodiments of this method, the disaggregating step is performed by trypsinizing the heterogeneous population of cells. In certain other embodiments of this method, the heterogeneous population of cells from step (1)(b) is plated in step (2)(a) at limiting dilution or at low density and subsequently removed using cloning cylinders, to arrive at individual cultures each of which originated from a single cell or small number of cells (oligoclonal). In further embodiments of this method, the limiting dilution is performed in multiwell dishes. In certain other embodiments of this method, the heterogeneous population of cells from step (1)(b) are plated in juxtaposition with feeder or inducer cells. In certain other embodiments of this method, the heterogeneous population of cells from step (1)(b) are plated as single isolated cells at low density in a semisolid media in step (2)(a). In certain other embodiments of this method, the heterogeneous population of cells from step (1)(b) are cultured in hanging drop culture. In certain other embodiments of this method, the heterogeneous population of cells from step (1)(b) are cultured as single isolated cells at low density in hanging drop culture in step (2)(a) and cultured in step (2)(b) as cell aggregates. In certain other embodiments of this method, the heterogeneous population of cells from step (1)(b) are cultured in step (2)(a) at low cellular density such that colonies of proliferating cells derived from a single cell can be easily identified and isolated using cloning cylinders or other similar means well known in the art and subsequently propagated in step (2)(b). In certain embodiments of this method, the pluripotent stem cells are differentiated in vitro, in vivo, or in ovo. In certain embodiments of this method, the heterogeneous population of cells forms a multicellular aggregate, such as an embryoid body. In certain embodiments of this method, the method of this invention further comprises the step of disaggregating the multicellular aggregate into single cells, by, for example, trypsinizing the multicellular aggregate. In certain embodiments of this method, the cells contained in a plurality of wells of step (1)(b) are documented by genotype or phenotype prior to step (2)(a), such as by photography, by immunocytochemistry or by hybridization of probes with RNA or cDNA transcript. In certain embodiments, the heterogeneous population of cells is not disaggregated prior to plating but clonal or oligoclonal growth originates from the original heterogeneous aggregate. In certain embodiments, the single cells and/or their progeny may be isolated as an oligoclonal population of cells, each of which have similar characteristics (as it is known that like cells often share morphology and have common cell adhesion molecules and adhere together). In certain embodiments, the pluripotent stem cells form embryoid bodies prior to being exposed to differentiation conditions. The parent cells may be pluripotent or may be totipotent.

This invention also provides a method for deriving desired cell types (“derived cells”) from parent pluripotent stem cells comprising the steps of:

(1) exposing said parent pluripotent stem cells in various differentiation conditions for various time periods resulting in a heterogeneous population of cells comprising cells with reduced differentiation potential than said parent pluripotent stem cells, wherein a plurality of said cells may have reduced differentiation potential than said parent pluripotent stem cells;
(2)(a) culturing said heterogeneous population of cells from step (1) in culture conditions wherein said single or small number of cells proliferate and the progeny of said single or small number of cells may be isolated as a clonal or oligoclonal culture of cells; wherein said heterogeneous population of cells comprising cells with reduced differentiation potential than the parent population may optionally be disaggregated to single cells prior to culturing, and
(2)(b) propagating said clonal population of cells of step (2)(a), resulting in said derived cells, wherein said cells are more uniform in differentiation potential and have reduced differentiation potential compared to the parent pluripotent stem cell population. The derived cells comprise cells that have the ability to differentiate further, i.e., they are not terminally differentiated cells. The parent cells may be pluripotent or may be totipotent. In certain embodiments, the cells in steps (2)(a) and (2)(b) are grown in the same medium, including the differentiation conditions, as the medium used in step (1) to differentiate the parent pluripotent stem cells. In certain embodiments of this method, the heterogeneous population of cells from step (1) are obtained by allowing said parent pluripotent stem cells to differentiate for various periods of time without disaggregation, i.e., for the cells to incubate in the differentiation conditions for various time periods before optionally disaggregating them. In a further embodiment of this method, the heterogeneous population of cells from step (1) is obtained by allowing said parent pluripotent stem cells to differentiate for various periods of time without disaggregation, and further, comprising the step of producing embryoid bodies using a variety of culture conditions for various time periods. In further embodiments of this method, the embryoid bodies are differentiated for various time periods. In certain embodiments of this method, the disaggregating step is performed by trypsinizing the heterogeneous population of cells. In certain other embodiments of this method, the heterogeneous population of cells from step (1) is plated in step (2)(a) at limiting dilution or at low density allowing isolation using cloning cylinders, to arrive at individual cultures each of which originated from a single cell or each of which originated from an oligoclonal number of cells. In further embodiments of this method, the limiting dilution is performed in multiwell dishes. In certain other embodiments of this method, the heterogeneous population of cells from step (2)(a) is plated in juxtaposition with feeder or inducer cells. In certain other embodiments of this method, the heterogeneous population of cells from step (1) are plated as single isolated cells at low density in a semisolid media in step (2)(a). In certain other embodiments of this method, the heterogeneous population of cells from step (1)(b) is cultured in hanging drop culture. In certain other embodiments of this method, the heterogeneous population of cells from step (1) is cultured as single isolated cells at low density in hanging drop culture in step (2)(a) and cultured in step (2)(b) as cell aggregates. In certain other embodiments of this method, the heterogeneous population of cells from step (1) is cultured in step (2)(a) at low cellular density such that colonies of proliferating cells derived from a single cell can be easily identified and isolated using cloning cylinders or other similar means well known in the art and subsequently propagated in step (2)(b). In certain embodiments of this method, the pluripotent stem cells are differentiated in vitro, in vivo, or in ovo. In certain embodiments of this method, the heterogeneous population of cells forms a multicellular aggregate, such as an embryoid body. In certain embodiments of this method, the method of this invention further comprises the step of disaggregating the multicellular aggregate into single cells, by, for example, trypsinizing the multicellular aggregate. In certain embodiments of this method, the cells contained in a plurality of wells of step (2)(a) are documented by genotype or phenotype prior to step (2)(b), such as by photography, by immunocytochemistry or by hybridization of probes with RNA or cDNA transcripts. In certain embodiments, the heterogeneous population of cells is not disaggregated prior to plating. In certain embodiments, the single cells and/or their progeny may be isolated as an oligoclonal population of cells, each of which have similar characteristics (as it is known that like cells stick together). In certain embodiments, the pluripotent stem cells first form embryoid bodies prior to being exposed to differentiation conditions.

In another embodiment of the invention, cells from the first differentiation step, but prior to the clonal or oligoclonal propagation step, are placed in growth media similar to or identical to that in which they will be clonally or oligoclonally expanded in order to increase the number of cells capable of propagating in the medium of the second step. This enrichment step allows an increased number and more predictable number of cells to proliferate in the final clonal or oligoclonal medium of the second step. In some cases where the medium of the initial differentiation step is identical to or similar to the medium in which the cells will be clonally or oligoclonally expanded, the enrichment step may also increase the number of proliferating cells such that the heterogeneous mixture may be cryopreserved, and in the event that the clonal or oligoclonal isolation yielded useful cell types, the cryopreserved heterogeneous mixture of cells may be thawed and used as a source of cells for clonal or oligoclonal isolation again. Therefore, in one embodiment, the enrichment step is part of the initial differentiation step in that the culture medium of the first differentiation step is identical to, or similar to, that of the second clonal or oligoclonal propagation step. Alternatively, the enrichment step may be a separate step. The cells may be initially differentiated in one medium, then the heterogeneous mixture of cells can be transferred at normal cell culture densities to a different medium of the second clonal or oligoclonal expansion step. The cells are cultivated in that medium in a separate step. After a period of time of 2-30 days (preferably 5-14 days) that allows for the percentage of cells capable of being propagated in the medium to be increased, the heterogeneous mixture of cells is then clonally or oligoclonally expanded as described herein.

The methods of this invention are to accelerate the isolation of novel cell strains (cell lines) from pluripotent stem cells. In certain embodiments, the methods of this invention are directed to the isolation of a large number of cell lines that are in various stages of differentiation or are differentiating. Some of these derived cells are terminally differentiated. Thus, it is an object of this invention to produce and isolate a large number of cell lines from pluripotent stem cells. Some of such cell lines are progenitor cells of various developmental lineages. Thus, in certain embodiments of this invention, it is a goal to isolate and propagate as many of the heterogeneous population of cells comprising cells with reduced differentiation potential than the starting parent pluripotent stem cells as possible.

In certain embodiments of this invention, the parent pluripotent stem cells or embryoid bodies derived therefrom are exposed to a variety of differentiating conditions. In certain embodiments of this invention, the plating step is performed at various time intervals after exposing said cells to the differentiating conditions.

In certain embodiments of this invention, the pluripotent stem cells are ES cells, EG cells, EC cells or ED cells. In certain embodiments, the starting pluripotent stem cells are teratomas. One way to form teratomas is as follows: human or non-human ES cells may be injected into an animal to induce three-dimensional growth, including but not limited to immunocompromised animals such as nude mice, or into SPF embryonated chick eggs. In certain embodiments of this invention, the pluripotent stem cells are human cells. In other embodiments, the pluripotent stem cells are non-human cells, such as mouse cells, non-human primate cells, rat cells, non-human mammalian cells such as bovine, porcine, equine, canine, or feline cells, etc.

In certain embodiments of this invention, the pluripotent stem cells are genetically modified such that the MHC genes are deleted (“nullizygotes” for MHC). In certain other embodiments of this invention, the pluripotent stem cells are genetically modified such that the MHC genes are first deleted and then alleles of the MHC gene family are restored such that these stem cells are hemizygous or homozygous for one allele of the MHC gene family.

In certain embodiments of this invention, the pluripotent stem cells are derived from the direct differentiation of embryonic cells (such as morula cells or inner mass cells) without the derivation of embryonic stem cell line.

In certain embodiments of this invention, the pluripotent stem cells are derived from blastomeres. For example, blastomere, morula, or ICM cells can be plated in step (1)(a) as are the other pluripotent stem cells of the present invention, and then clonal or oligoclonal cells can be isolated by following steps (1)(b) through (2)(b) as described herein where the pluripotent cells of the embryo yield clonal or oligoclonal cell lines without the intermediate step of ES cell line derivation.

In certain embodiments of this invention, the pluripotent stem cells are derived from the reprogramming of a somatic cell through the exposure of said somatic cell to the cytoplasm of an undifferentiated cell. In certain embodiments of this invention, the derived cells are endodermal cells, ectodermal cells or mesodermal cells, or cells of neural crest origin (the latter often designated ectodermal). In other embodiments of this invention, the derived cells are neuroglial precursor cells including definitive ectoderm and primitive neuroepithelium. In other embodiments of this invention, the derived cells are definitive endodermal cells such as hepatic cells or hepatic precursor cells, foregut, midgut, or hindgut endoderm, lung, pancreatic beta, or other endothermal precursor cells. In other embodiments of this method, the derived cells are chondrocyte, bone, or syovial precursor cells. In yet other embodiments of this invention, the derived cells are myocardial or myocardial precursor cells. In yet other embodiments of this invention, the derived cells are smooth muscle or skeletal muscle precursor cells including, but not limited to, somatic muscle precursor cells, muscle satellite stem cells and myoblast cells. In yet other embodiments of this invention, the derived cells are precursors of the branchial arches including those of the first branchial arch, such as mandibular mesenchyme, tooth, gingival fibroblast or gingival fibroblast precursor cells. In yet another embodiment of the invention, the derived cells are those of the intermediate mesoderm and precursors of kidney cells. In yet other embodiments of this invention, the derived cells are dermal fibroblasts with prenatal patterns of gene expression leading to scarless regeneration following wounding. In yet other embodiments of this invention, the derived cells are retinal precursor cells. In yet other embodiments of this invention, the derived cells are hemangioblasts.

This invention also provides isolated cells derived by the methods described above. This invention also contemplates genetically modifying these isolated cells.

In certain embodiments, the cells derived by the methods of this invention could be used as feeders or inducers on which other cells can be clonally expanded. In certain embodiments, the cell lines of this invention could be used as feeders or inducers in the first differentiation step (with or without the step of enrichment). One skilled in the art would know where particular factors are known to be useful in induction, and one can search for such factors in cell lines that express the mRNA for that factor.

In certain embodiments, the cell lines made by the methods of this invention may be incorporated into devices and this invention provides such devices. Many of the cell lines made by the methods of this invention secrete factor(s) that may be useful therapeutically. Such cells could be mitotically inactivated, and the mitotically inactivated cells may be applied to a number of matrices to make a tissue engineered construct where the cells survive for a period of time secreting the factor(s) and then die. In certain embodiments, the cells are irradiated to inactivate them. A typical irradiation protocol for this purpose (given cells in a free state) would involve exposing the cells to 20 to 50 Gy (2000 to 5000 rads; sometimes up to 100 Gy) from a Cs-137 or C0-60 source. In certain embodiments, a practical device configuration for releasing secreted factors would involve cell encapsulation. Another way to inactivate cells is by treating the cells with mitomycin C, as exemplified in Example 44. The cells can be encapsulated (or microencapsulated) collectively or as clusters or individually in porous implantable polymeric capsules. These can be made of a variety of substances, including but not limited to, polysaccharide hydrogels, chitosans, calcium or barium alginates, layered matrices of alginate and polylysine, poly(ethylene glycol) (PEG) polymers, polyacrylates (e.g., hydroxyethyl methacrylate methyl methacrylate), silicon, or polymembranes (e.g., acrylonitrile-co-vinyl chloride) in capillary-like, tube-like or bag-like configurations. Among the requirements for therapeutic utility are chemical definability, the ability to validate structure, stability, resistance to protein absorption, lack of toxicity, permeability to oxygen and nutrients as well as to the released therapeutic compounds, and resistance to antibodies or cellular attack. See, e.g., Orive et al. (2003) Nature Medicine 9(1):104-107 and Methods of Tissue Engineering, Eds Atalla, A. and Lanza, R. P. Academic Press, 2002.

Aspects of the present invention include a population of cells generated according to the methods described herein. In certain embodiments, the population of cells is a clonal progenitor cell line (e.g., a clonal embryonic progenitor cell line) that is capable of propagating in vitro for 20 doublings or more. In certain embodiments, the population of cells expresses a specific gene or gene subset (see, e.g., the cell lined described in Example 51, based on West et al., 2008, Regenerative Medicine vol. 3(3) pp. 287-308).

Aspects of the present invention include progenitor cell lines or groups of progenitor cell lines that exhibit specific gene expression patterns. The present invention provides a large number of such cells lines along with expression data for a large number of genes in each (see, e.g., Tables XX, XX1, XXII, XXIII, and XXIV). As such, the present invention provides progenitor cell lines that can be defined, categorized, and or grouped according to their gene expression pattern. The gene expression pattern is a term well known by those of ordinary skill in the art, and includes both relative gene expression (e.g., as compared to a control, e.g., a control gene in the same or different cell or cell line, or as compared to background detection as defined in the particular assay being employed (e.g., background fluorescence on a gene microarray)) or absolute gene expression (e.g., the amount of the gene product present in the cell). A gene expression pattern can include gene expression information for any number of genes, including 1, 2, 3, 5, 10, 20, 100, 1,000, 10,000, 100,000 or more genes. In certain embodiments, gene expression is based on mRNA levels present in the cells.

Aspects of the present invention include progenitor cell lines or groups of progenitor cell lines that produce specific factors (e.g., soluble growth factors) and/or inducing factors (e.g., factors that induce specific responses in cells, e.g., cell differentiation). As such, the present invention includes any specific progenitor cell line where the cell line can be defined by the specific factors it produces and/or does not produce. Cell lines may be categorized as producing specific factors by their gene expression pattern (e.g., mRNA levels as described above) and/or by direct analysis of the production of the factors themselves, e.g., ELISA assays for detecting the presence of soluble protein factors in culture supernatants or the use of flow cytometry to detect the presence of cell surface-associated factors. Any convenient method for the analysis of the production of factors by the cell lines according to aspects of the present invention may be used.

### BRIEF DESCRIPTION OF THE DRAWINGS

FIG. 1 shows a schematic drawing illustrating one experimental design for performing the differentiation step of pluripotent stem cells by subjecting said pluripotent stem cells to a variety or combination of differentiation conditions over time, leading to a heterogeneous population of cells, herein referred to as candidate cultures. In order to identify the individual candidate cultures (“CC”), each CC is assigned a reference position number (such as CC1-CC90).

FIG. 2 shows a schematic drawing illustrating one experimental design for performing the propagation step of the candidate cultures identified from FIG. 1. Under the propagation step, the individual candidate cultures are disaggregated to produce single cells and then subjected to an array of combinations of propagation conditions that promote cellular differentiation or propagation.

FIG. 3 shows colony growth visualized with crystal violet staining after two weeks of growth. FIG. 3A depicts the entire plate of colonies. Colonies that were removed from the plate with cloning cylinders were identified by the circular markings. FIG. 3B depicts colonies that were determined to be too close together to be separated. FIG. 3C depicts the typical colonies that were subsequently chosen for isolation. These discrete colonies were characterized as colonies with uniformly circular boundaries that were at this or greater distances apart from each other. See Example 13.

FIG. 4 depicts a representative phase contrast photograph of single cell-derived populations of cells (ACTC 2017, ACTC 2026 and ACTC 20230) in their primary colonies (P0) and after the fourth passage (P4). See Example 13.

FIG. 5 depicts a phase contrast photograph of dermal progenitor candidate Clone 8 (ACTC51/B2).

FIGS. 6A to 6F depict the relative pattern of gene expression of 17 different cell clones derived from Series 1 as described in Example 17. The cell clone numbers 1-17 along the horizontal axis represent the following cell lines: (1) ACTC61 or B30, (2) ACTC54 or B17, (3) ACTC52 or B29, (4) ACTC56 or B6, (5) 4-1, (6) 4-3, (7) B-10, (8) ACTC51 or B2, (9) ACTC53 or B7, (10) ACTC57 or B25, (11) ACTC58 or B11, (12) ACTC55 or B3, (13) ACTC50 or B26, (14) ACTC64 or 6-1, (15) ACTC62 or 2-2, (16) ACTC63 or 2-1, and (17) ACTC60 or 8-28. The cell clones in FIGS. 7-16, 18, 21 and 23 represent the same Series 1 cell lines. The expression of the following genes in each of the 17 cell clones was measured in FIG. 6: (a) dermo-1 (TWIST2), (b) dermatopontin (DPT), (c) PRRX2, (d) PEDF (SERPINF1), (e) AKR1C1, (f) collagen VI/alpha 3 (COL6A3), (g) microfibril-associated glycoprotein 2 (MAGP2), (h) GLUTS, (i) WISP2, (j) CHI3L1, (k) Odd-Skipped Related 2 (OSR2), (l) angiopoietin-like 2 (ANGPTL2), (m) RGMA, (n) EPHA5, (o) smooth muscle Actin Gamma 2 (ACTG2), (p) fibulin-1 (FBLN1), (q) LOXL4, (r) CD44 (the receptor for hyaluronic acid which promotes scarless wound repair), and (s) ADPRT (housekeeping gene for purposes of normalization). Values shown in the vertical axis of each of the histograms of the 17 cell clones of Series 1 represent the mean normalized relative fluorescent units (RFU) of the gene of interest. Values of approximately 100 RFU represent nonspecific background signal. The expression of these genes may be useful as markers to identify dermal fibroblast progenitor cells.

FIG. 7 depicts the relative expression of the SOX11 gene in the 17 different cell clones derived from Series 1 as described in Example 17. FIG. 7 illustrates that cell clone 1 of Series 1 as compared to some other cell clones of Series 1 express higher levels of the SOX11 gene. Values shown represent the normalized relative fluorescent units (RFU). See Example 18.

FIG. 8 depicts the relative expression of the CPE gene in the 17 different cell clones derived from Series 1 as described in Example 17. FIG. 8 illustrates that cell clones 1, 2, 4, 5, 6 and 7 of Series 1 express higher levels of the CPE gene as compared to some other cell clones of Series 1. Values shown represent the normalized relative fluorescent units (RFU). See Example 18.

FIG. 9 depicts the relative expression of the CPZ gene in the 17 different cell clones derived from Series 1 as described in Example 17. FIG. 9 illustrates that cell clones 8, 9, 10, 11, 13 and 14 of Series 1 express higher levels of the CPZ gene as compared to some other cell clones of Series 1. Values shown represent the normalized relative fluorescent units (RFU). See Example 18.

FIG. 10 depicts the relative expression of the C3 gene in the 17 different cell clones derived from Series 1 as described in Example 17. FIG. 10 illustrates that cell clones 8, 9, 10 and 12 of Series 1 express higher levels of the C3 gene compared to some other cell clones of Series 1. Values shown represent the normalized relative fluorescent units (RFU). See Example 18.

FIG. 11 depicts the relative expression of the MASP1 gene in the 17 different cell clones derived from Series 1 as described in Example 17. FIG. 11 illustrates that cell clones 8, 10, 11, 14, 15 and 16 of Series 1 express higher levels of the MASP1 gene as compared to some other clones of Series 1. Values shown represent the normalized relative fluorescent units (RFU). See Example 18.

FIG. 12 depicts the relative expression of the BF gene in the 17 different cell clones derived from Series 1 as described in Example 17. FIG. 12 illustrates that cell clones 10, 12, 13 and 14 of Series 1 express higher levels of the BF gene as compared to some other clones of Series 1. Values shown represent the normalized relative fluorescent units (RFU). See Example 18.

FIG. 13 depicts the relative expression of the FGFR3 gene in the 17 different cell clones derived from Series 1 as described in Example 17. FIG. 13 illustrates that cell clone 1 of Series 1 expresses higher levels of the FGFR3 gene as compared to some other cell clones of Series 1. Values shown represent the normalized relative fluorescent units (RFU). See Example 18.

FIG. 14 depicts the relative expression of the MYL4 gene in the 17 different cell clones derived from Series 1 as described in Example 17. FIG. 14 illustrates that cell clone 4 of Series 1 expresses higher levels of the MYL4 gene as compared to some other cell clones of Series 1. Values shown represent the normalized relative fluorescent units (RFU). See Example 18.

FIG. 15 depicts the relative expression of the MYH3 gene in the 17 different cell clones derived from Series 1 as described in Example 17. FIG. 15 illustrates that cell clone 9 of series 1 expresses higher levels of the MYH3 gene as compared to some other cell clones of Series 1. Values shown represent the normalized relative fluorescent units (RFU). See Example 18.

The clones referred to above are described in Example 17. Series 1 refers to the cell lines generated in Example 17.

FIGS. 16A to E depict the relative mRNA expression levels of various genes in the 17 cell clones derived from Series 1, as compared to the housekeeping ADPRT gene. The following gene markers were expressed: (a) actin gamma 2, (b) smooth muscle actin (ACTA2), (c) the endothelial receptor for angiopoietin-1 (TEK), (d) PLAP1, (e) tropomyosin-1 (TPM-1), (f) calponin-1 (CNN1), (g) dysferlin, (h) the unidentified gene LOC51063, (i) the oxidized low-density (lectin-like) receptor-1 (OLR1), (j) LRP2 binding protein (Lrp2bp), (k) MAGP2, (l) LOXL4, (m) MaxiK), and (n) ADPRT (shown for purposes of normalization). The expression of these genes may be useful as markers to identify smooth muscle progenitor cells. Based on the relative expression patterns illustrated in FIG. 16, cell clones 15-17 of Series 1 express unique markers of novel embryonic smooth muscle cell strains. Cell clones 15-17 and details relating to the markers are described in Example 21.

FIG. 17 depicts a phase contrast photographs of smooth muscle clonogenic cell lines produced from hES cell line ACT3. Clone 15 (ACTC62/2-2), clone 16 (ACTC63/2-1) and clone 17 (ACTC60/B-28) of Series 1 are shown after thawing at passage number 7. See Example 21.

FIGS. 18A to D depict the expression of HOX and other developmentally-regulated segmentation genes in identifying cell types in hES-derived cell clones 1-17 of Series 1. The expression of the following gene markers was measured in FIG. 18: (a) Dlx1, (b) Dlx2, (c) HOXD1, (d) HOXA2, (e) HOXA5, (f) HOXC6, (g) HOXD8, (h) HOXC10, (i) HOXA11 and (j) HOXD11. See Example 22.

FIG. 19 is a photograph of a representative clonogenic colony of candidate cells expressing a prenatal pattern of dermal fibroblast gene expression derived from embryoid bodies.

FIG. 20 is a photograph of a representative clonogenic colony of candidate epidermal keratinocyte cells expressing a prenatal pattern of gene expression derived from embryoid bodies as described in Example 24.

FIGS. 21A to F depict the relative pattern of gene expression of clone 8 as compared to the standard housekeeping ADPRT gene. The following genes were expressed in clone 8, consistent with clone 8 of series 1 being a dermal fibroblast progenitor cell: (a) dermo-1 (TWIST2), (b) dermatopontin (DPT), (c) PRRX2, (d) PEDF (SERPINF1), (e) AKR1C1, (f) collagen VI/alpha 3 (COL6A3), (g) microfibril-associated glycoprotein 2 (MAGP2), (h) fibulin-1 (FBLN1), (i) LOXL4, (j) CD44 (the receptor for hyaluronic acid which promotes scarless wound repair), (k) WISP2, (l) CHI3L1, (m) Odd-Skipped Related 2 (OSR2), (n) angiopoietin-like 2 (ANGPTL2), (o) RGMA, (p) EPHA5, (q) smooth muscle Actin Gamma 2 (ACTG2). The expression of the housekeeping ADPRT gene is depicted in (r) (the units for this gene on FIG. 21(r) are not relative units; they are absolute units on the y-axis). See Example 17.

FIG. 22 depicts a phase contrast photograph of dermal progenitor cells from clone 8 (ACTC51/132) of series 1. See Example 17.

FIGS. 23A to D depict the relative pattern of gene expression of 17 different cell clones derived from Series 1 as described in Example 17, as compared to the standard housekeeping ADPRT gene. The expression of the following genes was measured: (a) HOXA2, (b) HOXB-2, (c) SOX11, (d) ID4, (e) FOXC1, (f) Cadherin-6, (g) PTN, (h) SLITRK3 and (i) CRYAB. The expression of the housekeeping ADPRT gene is depicted in (j) (shown for purposes of normalization). The expression of these genes may be useful as markers to identify cranial neural crest progenitor cells. See Example 26.

FIG. 24 depicts a phase contrast photograph of single cell-derived cranial neural crest cells (clone 1; also referred to as ACTC61/B30) of Series 1 at passage 7 derived from the human ES cell line ACT3. See Example 26.

FIG. 25 depicts the relative expression of the VEGFC gene in the 17 different cell clones derived from Series 1 as described in Example 17.

FIG. 26 depicts the differential gene expression of prohormone convertase PCSK1N, PCSK5 and PCSK9 in 28 clones that are derived from hES cell lines, generated from series 2 as described in Example 26. RFU on the y-axis represents the relative fluorescent units. The 28 clones are shown in the x-axis.

In FIGS. 6-18, 21, 23 and 25, the y-axis represents relative units and clones 1-17 of Series 1 (see examples 17, 18, 21, 22, 25 and 26) are shown in the x-axis.

FIGS. 27A to J depict a table of the microRNA profiles of eleven cell lines generated according to the methods of this invention. The no template control (NTC) serves as the control. See Example 29.

FIG. 28 depicts the real-time quantitation method termed looped-primer RT-PCR used for sensitive and accurate detection of microRNAs present in a sample. The method involves two steps: stem-loop RT followed by real-time PCR. See Example 29.

FIGS. 29A to F depict a table of the microRNA profiles of summarizes the results of cellular miRNA levels in the H9 human embryonic stem cell line, the Fb-p1 fibroblast cell line and nine cell lines differentiated from parental human embryonic stem cells. The unique miRNA profiles (highlighted in bold) are apparent for all cell lines tested here. See Example 29.

FIG. 30 depicts a schematic representation of real-time PCR-based 330-plex microRNA expression profiling method as described in Example 30.

FIG. 31 illustrates a robotic platform which may be used to perform the methods of the invention.

FIGS. 32-42 and Supplementary tables are from West et al., 2008, Regenerative Medicine vol. 3(3) pp. 287-308, which is incorporated by reference herein in its entirety (See Example 51).

FIG. 32. Two-step multiplex hEP derivation protocol. (a) In the first step hES cells are exposed to an array of differentiation conditions to generate diverse and heterogeneous subpopulations of embryonic progenitor cell types designated candidate cultures (CCs); (b) In the second step each CC subpopulation is plated at clonal densities in another array of media and growth factors to identify EP cell clones capable of long-term propagation.

FIG. 33. Clonogenicity of hES-EPs derived by in situ colony differentiation. (a) Crystal violet stained 150 mm dish following the removal of selected clones; (b) Clones too close or lacking circular periphery and therefore not selected for subculture; (c) Minimum separation in colonies selected for subculture; (d) Clone B30 (ACTC61) in the original colony (P0) (100×); (c) Clone B30 (ACTC61) after four passages (P4) (100×).

FIG. 34. Genes with highly constitutive expression in diverse hES-derived cells. The relative expression of the genes RPL23 (yellow triangles), RPS10 (magenta squares), ATP5O (light green Xs), ATP5F1 (pink Xs), and PRDX5 (red squares) from the Illumina 1 data set (Supplementary Table I from West et al., 2008, Regenerative Medicine vol. 3(3) pp. 287-308, which is incorporated by reference herein in its entirety) displayed less variability among the isolated hEP cell lines compared to the expression of the commonly-used constitutive marker GAPD (purple trapezoids).

FIG. 35. hEP cells lack ES markers while retaining the expression on early developmentally-regulated genes. Histograms show the normalized, hierarchically clustered combined data expressed as relative fluorescence units (RFU) for select genes in the combined Illumina 1 and 2 data sets. The parental hES cell line H9 is included in biological replicate in the first two lanes.

FIG. 36: Abbreviated heat map of common gene sequences on Illumina 1 and 2 platforms for hierarchically clustered cell lines. hES cells and derived hEP cell clones, normalized and hierarchically clustered, with the resulting dendrogram and heat map. Relatively highly expressed genes are shown in red and genes not expressed are blue. The parental hES cell line H9 is included in biological replicate in the first two columns.

FIG. 37. Heat map of selected developmentally-regulated homeobox gene expression in hEP cell lines. Normalized and combined Illumina 1 and 2 data for select members of the DLX, MEOX, HOX, LIM, MSX, BAPX, PRRX, GSC, IRX, SOX, PITX, and FOX homeobox genes that were differentially expressed in the clones were hierarchically clustered and plotted as a heat map. Relatively highly expressed genes are shown in red and genes not expressed are blue.

FIG. 38: NMF plot of cell clones analyzed on the Illumina platform. Normalized and combined Illumina 1 and 2 gene expression data where k=140 is shown. Red squares correspond to cells placed in the same group. Blue squares show no correlation. Cell line group assignments and cell line identification is shown in Supplementary Table 1 (from West et al., 2008, Regenerative Medicine vol. 3(3) pp. 287-308, which is incorporated by reference herein in its entirety).

FIG. 39. Immunocytochemical confirmation of microarray gene expression data in cells lines displaying neural crest and endodermal markers. (a-f) Staining of the cell line 7PEND24 (ACTC283) with: (a) anti-NES antibody (100×); (b) anti-NES (400×); (c) isotype control antibody (400×); (d) anti-CNTN6 antibody (100×); (e) anti-CNTN6 (400×); (f) isotype control antibody (100×); (g-l) Staining of the cell line M10 (ACTC103) with: (g) anti-AFP antibody (100×); (h) anti-AFP antibody (400×); (i) isotype control antibody (100×); (j) anti-KRT20 antibody (100×); (k) anti-KRT20 (400×); and (l) isotype control antibody (100×). Scale bar=10 μm.

FIG. 40. Immunocytochemical confirmation of microarray gene expression data in cells lines displaying mesodermal and ectodermal markers. (a-f) Staining of the cell line SK17 (ACTC162) with: (a) anti-MYH3 antibody (100×); (b) anti-MYH3 (400×); (c) isotype control antibody (100×); (d) anti-NES antibody (100×); (e) anti-NES (400×); and (f) isotype control antibody (100×); (g-l) Staining of the cell line E68 (ACTC207) with: (g) anti-SNAP25 (100×); (h) anti-SNAP25 (400×); (i) isotype control antibody (100×); (j-l) Staining of the cell line E68 (ACTC) with: (j) anti-CNTN6 antibody (100×); (k) anti-CNTN6 (400×); and (l) isotype control antibody (100×). Scale bar=10 μm.

FIG. 41. Induction of neuronal differentiation. (a) Cell line E68 (ACTC207) at passage 19 in the derivation media (100×); (b) E68 at 57 days in neural induction medium (arrow: structures resembling compacted neuroepithelium) (200×); (c) E68 at 57 days in neural induction medium (arrow: structures resembling growth cones) (400×); (c) E68 at 57 days in neural induction medium (arrow: synapse-like structures) (400×).

FIG. 42. Proliferative potential of hEP cell lines. (a) Growth curves of the cell lines EN13 (filled diamond), SK17 (filled square), SM28 (filled triangle), and SM22 (cross), compared to neonatal foreskin fibroblasts (Xgene) (open circle). (b) TRF analysis of DNA from hES cells (H9), compared to the cell lines at various passage numbers; (c) Scatter plots of mean TRF length vs. population doubling number.

FIG. 43: Confirmation of select relative gene expression as measured by microarray by qPCR. Comparison of qPCR (light blue) and bead array values (gray) are displayed for A) FOXF1, B) FOXG1B, C) SOX4, and D) HOXC6 in selected cell lines.

FIG. 44: Stability scores for NMF analysis with k values of 100-145. The stability score (cophenetic correlation coefficient) is plotted against chosen partition numbers (k values) ranging 100-145. The arrow points to the highest stability score that did not break known biological and technical replicates into separate groups.

FIG. 45: TRAP assay results for select cell lines. TRAP ladders for telomerase positive hES cells (H9) are shown along with the hES-derived cell lines SM28, SK17, EN13, SM22, and the control dermal fibroblast Xgene. Cells are shown at different passage numbers. Controls include samples with no cell lysate (negative TRAP result), heat denatured telomerase positive sample (negative TRAP result), and RNAse-treated telomerase positive extract (negative TRAP result).

It is noted here that all Supplementary Information from West et al., 2008, Regenerative Medicine vol. 3(3) pp. 287-308 is incorporated by reference herein in its entirety. A brief list of is provided below.

Supplementary Figure A3: Dendrograms and heat map of all genes in common between Illumina 1 and 2 platforms. RFU values from the probe sequences identical in Illumina 1 and Illumina 2 microarrays were used to generate data quantile normalized values between the two platforms. The values were then hierarchically clustered and a heat map was generated to show cell lines that express similar relative levels of genes (horizontal axis), and gene families that show similar patterns of expression in the cell lines (vertical axis). Relatively high levels of expression are displayed red and relatively low levels of expression are blue.

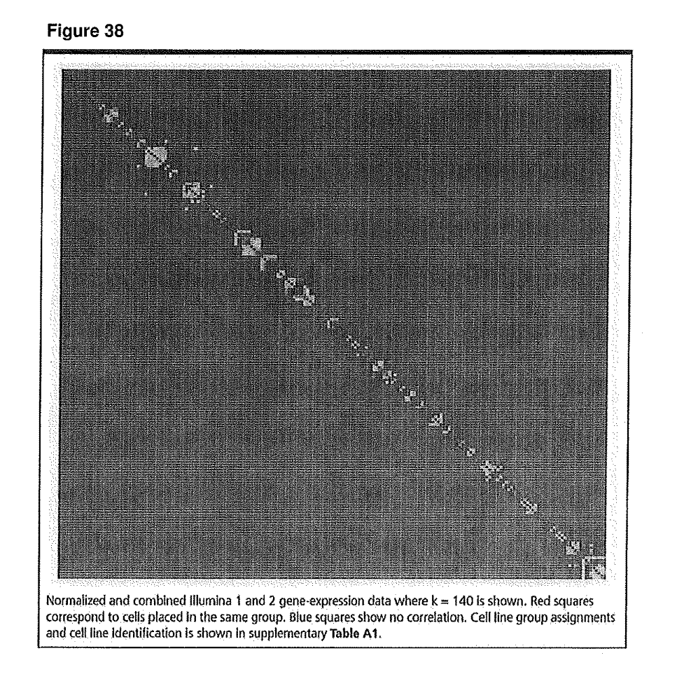

Supplementary Table I (from West et al., 2008, Regenerative Medicine vol. 3(3) pp. 287-308, which is incorporated by reference herein in its entirety): Collated data related to individual cell lines. Data relating to the parental hES cell line, ACTC number, common cell line name, methods of differentiation as either in situ differentiation or as embryoid bodies, medium used in the growth and differentiation of embryoid bodies, propagation medium (either one or two serial media), microarray analysis platform, and NMF group assignments as group identification number and order in FIG. 38 are tabulated.

Supplementary Table II (from West et al., 2008, Regenerative Medicine vol. 3(3) pp. 287-308, which is incorporated by reference herein in its entirety): Normalized annotated gene expression in cells analyzed on Illumina 1 microarrays. RFU values for cell lines analyzed on the Illumina 1 microarray platform were normalized by quantile normalization and rank ordered in decreasing values of (highest recorded RFU value for any cell line−lowest RFU value for any cell line)/mean RFU value for all cell lines. As a result, markers most differentially expressed are preferentially listed toward the top of the spreadsheet. Cells are displayed in a horizontal order corresponding to hierarchical clustering.

Supplementary Table III (from West et al., 2008, Regenerative Medicine vol. 3(3) pp. 287-308, which is incorporated by reference herein in its entirety): Normalized gene expression in cells analyzed on Illumina 2 microarrays. RFU values for cell lines analyzed on the Illumina 2 microarray platform are displayed as analyzed in the same manner as Supplementary Table I.

Supplementary Table IV (from West et al., 2008, Regenerative Medicine vol. 3(3) pp. 287-308, which is incorporated by reference herein in its entirety): Normalized gene expression in cells analyzed on Affymetrix microarrays. RFU values for cell lines analyzed on the Affymetrix microarray platform are displayed as analyzed in the same manner as Supplementary Table I.

Supplementary Table V (from West et al., 2008, Regenerative Medicine vol. 3(3) pp. 287-308, which is incorporated by reference herein in its entirety): Genes expressed at relatively high levels in individual hEP cell lines. Gene RFU values for the 45 most differentially expressed genes in individual cell lines were rank ordered in decreasing order with the ratio of RFU value of the gene in an individual cell line/mean RFU value of that gene in all cell lines analyzed on the same microarray platform. In addition to normalized RFU values, expression relative to GAPD are displayed as a standard of absolute levels of expression.

Supplementary Table VI (from West et al., 2008, Regenerative Medicine vol. 3(3) pp. 287-308, which is incorporated by reference herein in its entirety): CD Antigen genes expressed at relatively high or low values in individual hEP cell lines. RFU values for 20 CD antigen genes differentially expressed at relatively higher or lower levels than the mean RFU value of that gene in all cell lines analyzed on the same microarray platform. Ratios of the RFU value for a specific gene in a particular cell line/average RFU values for that gene in all cell lines are displayed under the heading Ave Ratio.

Supplementary Table VII (from West et al., 2008, Regenerative Medicine vol. 3(3) pp. 287-308, which is incorporated by reference herein in its entirety): Genes encoding secreted proteins expressed at relatively high levels in individual hEP cell lines. Gene RFU values for the most differentially expressed genes in individual cell lines were rank ordered in decreasing order with the ratio of (RFU value of the gene in an individual cell line−lowest RFU value observed in any cell line)/mean RFU value of that gene in all cell lines analyzed on the same microarray platform.

Supplementary Table VIII (from West et al., 2008, Regenerative Medicine vol. 3(3) pp. 287-308, which is incorporated by reference herein in its entirety): Confirmation of representative secreted factors by ELISA. Genes for selected secreted factors were assayed by ELISA showing that cell lines displaying relatively high levels of secreted protein RNA were also those that showed relatively high levels of assayable protein.

### DETAILED DESCRIPTION OF THE INVENTION

### Abbreviations

| | |
|---|---|
| AFP | Alpha fetoprotein |
| BMP | Bone Morphogenic Protein |
| BRL | Buffalo rat liver |
| BSA | Bovine serum albumin |
| CD | Cluster Designation |
| cGMP | Current Good Manufacturing Processes |
| CNS | Central Nervous System |
| DMEM | Dulbecco's modified Eagle's medium |
| DMSO | Dimethyl sulphoxide |
| DPBS | Dulbecco's Phosphate Buffered Saline |
| EC | Embryonal carcinoma |
| EC Cells | Embryonal carcinoma cells; hEC cells are human embryonal carcinoma cells |
| ECM | Extracellular Matrix |
| ED Cells | Embryo-derived cells; hED cells are human ED cells |
| EDTA | Ethylenediamine tetraacetic acid |
| EG Cells | Embryonic germ cells; hEG cells are human EG cells |
| ES Cells | Embryonic stem cells; hES cells are human ES cells |
| FACS | Fluorescence activated cell sorting |
| FBS | Fetal bovine serum |
| GMP | Good Manufacturing Practices |
| hED Cells | Human embryo-derived cells |
| hEG Cells | Human embryonic germ cells are stem cells derived from the primordial germ cells of fetal |
| | tissue. |
| hiPS Cells | Human induced pluripotent stem cells are cells with properties similar to hES cells obtained from |
| | somatic cells after exposure to hES-specific transcription factors such as SOX2, KLF4, OCT4, MYC, or NANOG, LIN28, |
| | OCT4, and SOX2. |
| HSE | Human skin equivalents are mixtures of cells and biological or synthetic matrices manufactured |
| | for testing purposes or for therapeutic application in promoting wound repair. |
| ICM | Inner cell mass of the mammalian blastocyst-stage embryo. |
| iPS Cells | Induced pluripotent stem cells are cells with properties similar to hES cells obtained from |
| | somatic cells after exposure to ES-specific transcription factors such as SOX2, KLF4, OCT4, MYC, or NANOG, LIN28, |
| | OCT4, and SOX2. |
| LOH | Loss of Heterozygosity |
| MEM | Minimal essential medium |
| NT | Nuclear Transfer |
| PBS | Phosphate buffered saline |
| PS fibroblasts | Pre-scarring fibroblasts are fibroblasts derived from the skin of early gestational skin or derived |
| | from ED cells that display a prenatal pattern of gene expression in that they promote the rapid healing of dermal wounds |
| | without scar formation. |
| RA | Retinoic acid |
| RFU | Relative Fluorescence Units |
| SCNT | Somatic Cell Nuclear Transfer |
| SFM | Serum-Free Medium |
| SPF | Specific Pathogen-Free |
| SV40 | Simian Virus 40 |
| Tag | Large T-antigen |
| T-EDTA | Trypsin EDTA |

### DEFINITIONS

The term “analytical reprogramming technology” refers to a variety of methods to reprogram the pattern of gene expression of a somatic cell to that of a more pluripotent state, such as that of an ES, ED, EC or EG cell, wherein the reprogramming occurs in multiple and discrete steps and does not rely simply on the transfer of a somatic cell into an oocyte and the activation of that oocyte (see U.S. application Nos. 60/332,510, filed Nov. 26, 2001; Ser. No. 10/304,020, filed Nov. 26, 2002; PCT application no. PCT/US02/37899, filed Nov. 26, 2003; U.S. application No. 60/705,625, filed Aug. 3, 2005; U.S. application No. 60/729,173, filed Aug. 20, 2005; U.S. application No. 60/818,813, filed Jul. 5, 2006, PCT/US06/30632, filed Aug. 3, 2006, the disclosure of each of which is incorporated by reference herein).

The term “cellular reconstitution” refers to the transfer of a nucleus of chromatin to cellular cytoplasm so as to obtain a functional cell.

The term “cytoplasmic bleb” refers to the cytoplasm of a cell bound by an intact or permeabilized but otherwise intact plasma membrane, but lacking a nucleus.

The term “pluripotent stem cells” refers to animal cells capable of differentiating into more than one differentiated cell type. Such cells include hES cells, hED cells, HIPS cells, hEG cells, hEC cells, and adult-derived cells including mesenchymal stem cells, neuronal stem cells, and bone marrow-derived stem cells. Pluripotent stem cells may be genetically modified or not genetically modified. Genetically modified cells may include markers such as fluorescent proteins to facilitate their identification within the egg.

The term “primordial stem cells” refers collectively to pluripotent stem cells capable of differentiating into cells of all three primary germ layers: endoderm, mesoderm, and ectoderm, as well as neural crest. Therefore, examples of primordial stem cells would include but not be limited by hES, hED, hiPS, and hEG cells.

The term “embryonic stem cells” (ES cells) refers to cells derived from the inner cell mass of blastocysts, blastomeres, or morulae that have been serially passaged as cell lines while maintaining an undifferentiated state (e.g. expressing TERT, OCT4, and SSEA and TRA antigens specific for ES cells of the species). The ES cells may be derived from fertilization of an egg cell with sperm or DNA, nuclear transfer, parthenogenesis, or by means to generate hES cells with hemizygosity or homozygosity in the MHC region. The term “human embryonic stem cells” (hES cells) refers to human ES cells.

The term “colony in situ differentiation” refers to the differentiation of colonies of hES, hEG, hiPS, human EC or hED cells in situ without removing or disaggregating the colonies from the culture vessel in which the colonies were propagated as undifferentiated stem cell lines. Colony in situ differentiation does not utilize the intermediate step of forming embryoid bodies, though embryoid body formation or other aggregation techniques such as the use of spinner culture may nevertheless follow a period of colony in situ differentiation.

The term “direct differentiation” refers to process of differentiating blastomere cells, morula cells, ICM cells, ED cells, or somatic cells reprogrammed to an undifferentiated state directly without the intermediate state of propagating undifferentiated stem cells such as hES cells as undifferentiated cell lines.

The term “human embryo-derived” (“hED”) cells refers to blastomere-derived cells, morula-derived cells, blastocyst-derived cells including those of the inner cell mass, embryonic shield, or epiblast, or other totipotent or pluripotent stem cells of the early embryo, including primitive endoderm, ectoderm, and mesoderm and their derivatives, but excluding hES cells that have been passaged as cell lines. The hED cells may be derived from fertilization of an egg cell with sperm or DNA, nuclear transfer, chromatin transfer, parthenogenesis, analytical reprogramming technology, or by means to generate hES cells with hemizygosity or homozygosity in the HLA region.

The term “human embryonic germ cells” (hEG cells) refer to pluripotent stem cells derived from the primordial germ cells of fetal tissue or maturing or mature germ cells such as oocytes and spermatogonial cells, that can differentiate into various tissues in the body. The hEG cells may also be derived from pluripotent stem cells produced by gynogenetic or androgenetic means, i.e., methods wherein the pluripotent cells are derived from oocytes containing only DNA of male or female origin and therefore will comprise all female-derived or male-derived DNA (see U.S. application Nos. 60/161,987, filed Oct. 28, 1999; Ser. Nos. 09/697,297, filed Oct. 27, 2000; 09/995,659, filed Nov. 29, 2001; 10/374,512, filed Feb. 27, 2003; PCT application no. PCT/US100/29551, filed Oct. 27, 2000; the disclosures of which are incorporated herein in their entirety).

The term human iPS cells refers to cells with properties similar to hES cells, including the ability to form all three germ layers when transplanted into immunocompromised mice wherein said iPS cells are derived from cells of varied somatic cell lineages following exposure to hES cell-specific transcription factors such as KLF4, SOX2, MYC, and OCT4 or the factors SOX2, OCT4, NANOG, and LIN28. Said iPS cells may be produced by the expression of these gene through vectors such as retrovial vectors as is known in the art, or through the introduction of these factors by permeabilization or other technologies as described in PCT application number PCT/US2006/030632, filed on Aug. 3, 2006; U.S. application Ser. No. 11/989,988; PCT Application PCT/US2000/018063, filed on Jun. 30, 2000; U.S. Application Ser. No. 09,736,268 filed on Dec. 15, 2000; U.S. Application Ser. No. 10/831,599, filed Apr. 23, 2004; and U.S. Patent Publication 20020142397 (application Ser. No. 10/015,824, entitled “Methods for Altering Cell Fate”); U.S. Patent Publication 20050014258 (application Ser. No. 10/910,156, entitled “Methods for Altering Cell Fate”); U.S. Patent Publication 20030046722 (application Ser. No. 10/032,191, entitled “Methods for cloning mammals using reprogrammed donor chromatin or donor cells”); and U.S. Patent Publication 20060212952 (application Ser. No. 11/439,788, entitled “Methods for cloning mammals using reprogrammed donor chromatin or donor cells” all of which are incorporated herein by reference in their entirety.

The term “histotypic culture” refers to cultured cells that are aggregated to create a three-dimensional structure with tissue-like cell density such as occurs in the culture of some cells over a layer of agar or such as occurs when cells are cultured in three dimensions in a collagen gel, sponge, or other polymers such as are commonly used in tissue engineering.

The term “clonal” refers to a population of cells obtained the expansion of a single cell into a population of cells all derived from that original single cells and not containing other cells.

The term “oligoclonal” refers to a population of cells that originated from a small population of cells, typically 2-1000 cells, that appear to share similar characteristics such as morphology or the presence or absence of markers of differentiation that differ from those of other cells in the same culture. Oligoclonal cells are isolated from cells that do not share these common characteristics, and are allowed to proliferate, generating a population of cells that are essentially entirely derived from the original population of similar cells.

The term “pooled clonal” refers to a population of cells obtained by combining two or more clonal populations to generate a population of cells with a uniformity of markers such as markers of gene expression, similar to a clonal population, but not a population wherein all the cells were derived from the same original clone. Said pooled clonal lines may include cells of a single or mixed genotypes. Pooled clonal lines are especially useful in the cases where clonal lines differentiate relatively early or alter in an undesirable way early in their proliferative lifespan.

The term “differentiated cells” when used in reference to cells made by methods of this invention from pluripotent stem cells refer to cells having reduced potential to differentiate when compared to the parent pluripotent stem cells. The differentiated cells of this invention comprise cells that could differentiate further (i.e., they may not be terminally differentiated).

The term “organotypic culture” refers to cultured cells that are aggregated to create a three-dimensional structure with tissue-like cell density such as occurs in the culture of some cells over a layer of agar, cultured as teratomas in an animal, otherwise grown in a three dimensional culture system but wherein said aggregated cells contain cells of different cell lineages, such as, by way of nonlimiting examples, the combination of epidermal keratinocytes and dermal fibroblasts, or the combination of parenchymal cells with their corresponding tissue stroma, or epithelial cells with mesenchymal cells.

The term embryonal carcinoma (“EC”) cells, including human EC cells, refers to embryonal carcinoma cells such as TERA-1, TERA-2, and NTera-2. EC cells are well known in the art.

The term “cell expressing gene X”, “gene X is expressed in a cell” (or cell population), or equivalents thereof, means that analysis of the cell using a specific assay platform provided a positive result. The converse is also true (i.e., by a cell not expressing gene X, or equivalents, is meant that analysis of the cell using a specific assay platform provided a negative result). Thus, any gene expression result described herein is tied to the specific probe or probes employed in the assay platform (or platforms) for the gene indicated.

This invention provides methods for the derivation of cells that are derived from a single cell (clonal) or a small number of similar cells (oligoclonal) differentiated, or in the process of differentiating, from pluripotent stem cells, wherein said single cells or oligoclonal cells are propagated to produce a population of cells, a population being two or more cells, under propagation conditions identified by means of screening a panel of conditions including, but not limited to, combinations of growth factors, extracellular components, conditioned media, hormones, ion concentrations, and co-culture with inducing or feeder cell types. This invention also provides formulation and use of the cells derived from the methods of this invention as well as engineered tissues made of such cells. Certain embodiments of this invention are described in the summary of the invention section and will not be repeated in this detailed description section.

The cells of this invention are differentiated from, or in the process of differentiating from, pluripotent stem cells, which could be any pluripotent stem cells. In some embodiments, the pluripotent stem cells include hES, hEG, hiPS, hEC and hED cells, as well as pluripotent stem cells derived from the developing embryo such as those of the first eight weeks of human embryonic development including, but not limited to, pluripotent endodermal, mesodermal, or ectodermal progenitor cells. In some embodiments, the pluripotent stem cells may be derived from human or nonhuman embryonic or fetal tissues.

While techniques to differentiate hES cells into several differentiated states have been described, and whereas the use of clonogenic assays have been described for use in assaying the proliferative potential of bone marrow hematopoietic and stromal cells, for purifying some mixtures of cells, or otherwise characterizing said cells, the present invention uniquely describes the novel method of deriving populations of two or more, preferably one hundred or more, cells, from a single cell (clonal) or a small number of similar cells (oligoclonal) differentiated from, or in the process of differentiating from, embryonic pluripotent stem cells such as hES, hEG, hiPS, hEC, hED cells or other pluripotent embryonic stem cells such as primitive endoderm, mesoderm, or ectodermal cells, wherein the resulting single cell-derived or oligoclonal population of cells can be documented not to have contaminating cells from the original pluripotent stem cells, wherein the resulting single cell-derived or oligoclonal population of cells is isolated from a heterogeneous population and can be used in cell therapy, research, for the isolation of novel extracts with therapeutic utility, or for the derivation of ligands that specifically bind to said cells.

The present invention also provides a means of identifying single cell-derived or oligoclonal populations of cells of this invention capable of scalability. This invention also provides methods for identifying conditions for the propagation of said cells, for characterizing the differentiated state of said cells, and for identifying single cell-derived or oligoclonal populations of cells capable of being stably engrafted after transplantation.

In one aspect of the invention, the method provides a means of identifying single cell-derived populations of cells of this invention with a pattern of gene expression corresponding to that of an animal of the same species in the prenatal state in vivo, as well as identifying conditions for the propagation of said cells.

In one aspect of the invention, the method provides a means of identifying the single cell-derived populations of cells of this invention using flow cytometry or analogous affinity-based cell sorting technology such as magnetic bead sorting, and the further characterization of these cells' gene expression, phenotype and stability. The resulting suspension of sorted cells may then be plated at a density of a single cell per well for colony formation and subsequent clonal expansion. In some case, the cell plating step may be accomplished using an automated cell deposition device (“ACDU”). The use of flow cytometry is particularly useful where said cell of this invention is rarely present in the original heterogenous mixture of cells or where said cell of this invention has only limited capacity to proliferate after clonal or oligoclonal isolation. Moreover, a larger number of starting cells can be isolated to increase the final yield.

In another aspect of the invention, the complexity of the initial heterogenous mixture of cells that results from the first step may be reduced to concentrate cell types of interest by sorting cells using antigens that are expected to be on the desired cell type or family of cell types or by genetically modifying the parent pluripotent stem cells with expression DNA constructs that comprise a promoter and a marker gene such as GFP, such that the particular gene is expressed in the cell type or family of cell types that is desired, allowing such cells to be identified and isolated.

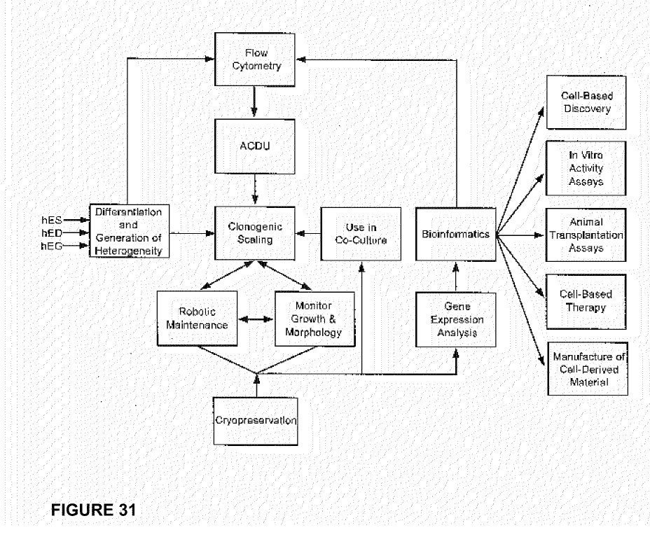

In another aspect of the invention, the methods of the invention may be automated, for example, by using robotic manipulation. In certain embodiments, cells may be expanded clonally or oligoclonally via robotic means in a variety of media, extracellular matrices, or co-cultured cells. In certain embodiments, robotic automation may also be used to monitor cell growth. In certain other embodiments, robotic automation may be used to culture and propagate cells made by methods of this invention, for example, passaging, feeding, and cryopreserving said cells, with generated information being stored in a computer database. This enables the reproducible production of desired cell types and may be useful in a research setting where a large number of culture conditions are assayed. Robotic automation of the methods of this invention may also be useful in personalized medicine where the robotic platform is combined with the cells from a patient and wherein each patient has customized differentiated cells produced. Components of such a robotic platform are illustrated in FIG. 31.

In one aspect of the invention, the method comprises the steps of deriving differentiated or differentiating cells by differentiating pluripotent stem cells for varying periods of time in vitro, in vivo, or in ovo, with or without an intermediate step of forming multicellular aggregates such as embryoid bodies, and distributing the differentiated cells in cell culture conditions wherein the cells are cultured attached to a substrate at such a low density that subsequent cultures are composed of colonies of cells derived from what was originally a single cell. In the case where multicellular aggregates such as embryoid bodies are formed, there may be a step to separate the aggregates into single cells, such as by trypsinizing the aggregates.

In another aspect of the invention, the method comprises the steps of deriving cells differentiated at various periods of time from pluripotent stem cells (such as hES cells), and culturing such differentiated or differentiating cells at low density in a semisolid media such that subsequent culture can identify colonies of cells derived from what was originally a single cell, wherein said differentiated or differentiating cells are cultured in combinations of various culture media (including, but not limited to, media conditioned in the presence of various cell types), growth factors, ambient gas concentrations, and extracellular matrices.

In certain embodiments, the differentiated cells or differentiating cells made by the methods of this invention are derived from a single cell that is documented by photography or other means of identification, such as immunocytochemical means or hybridization of probes with RNA or cDNA transcripts, to be a cell of a certain differentiated state such that it is not an ES cell in order to reduce the potential of transplanting undesired cells, such as undifferentiated cells including ES cells, into the animal or human in need of cell-based therapy. The lack of contaminating ES cells in the differentiated cell or differentiating cultures made by the methods of this invention eliminates the potential risk of tumor-forming ES cells. It has previously been known that ES-derived cells may have the capability to form tumors, as evidenced by the existence of cancer stem cells. In contrast, the lack of contaminating ES cells in the differentiated cell or differentiating cell cultures made by the methods of this invention eliminates such tumor-forming ES cells. To confirm this, for example, the tumor-forming ability of hES-derived clonal cell lines of Series 1 generated by the methods of this invention was compared with hES cells. When hES-derived clonal cell lines of Series 1 of the present invention or hES cells were injected intramuscularly or subcutaneously into the rear legs of SCID mice, large teratomas (approximately one cm) were observed only in hES-injected mice at the site of injection three months later. However, no evidence of tumors was observed in the animals injected with hES-derived clonal cell lines of Series 1 of the present invention. No signs of malignancy, edema, erythema, or other pathology were observed at the site of injection or in any of the analyzed tissues in animals injected with hES-derived clonal cell lines of Series 1 of the present invention.

In another aspect of the invention, the method comprises deriving 100 or more cells from a single differentiated cell, or a cell in the process of differentiating, said cell resulting from differentiating a pluripotent stem cell, such as a hES cell, wherein the pluripotent stem cell is genetically modified to delete genes from the MEW gene family or cells wherein genes of the MHC gene family are first removed and then alleles of the MHC gene family are restored such as to make hemizygous or homozygous stem cells (see U.S. application Ser. Nos. 10/445,195, filed May 27, 2003; 60/729,173, filed Oct. 20, 2005, the disclosures of which are incorporated by reference).

In another aspect of the invention, the method comprises the derivation of 100 cells or more from a single differentiated cell differentiated from a pluripotent stem cell, or from a cell in the process of differentiating from a pluripotent stem cell such as a hED cell, wherein the pluripotent stein cell is derived from the direct differentiation of an embryonic cell or cells without the derivation of a human ES cell line.

In another aspect of the invention, the method comprises the derivation of 100 cells or more from a single differentiated cell or a cell in the process of differentiating from a pluripotent stem cell such as a hES cell wherein the hES cell line is derived from a single blastomere. The pluripotent embryonic stem cells can also be generated from a single blastomere removed from an embryo without interfering with the embryo's normal development to birth. See U.S. application Nos. 60/624,827, filed Nov. 4, 2004; 60/662,489, filed Mar. 14, 2005; 60/687,158, filed Jun. 3, 2005; 60/723,066, filed Oct. 3, 2005; 60/726,775, filed Oct. 14, 2005; Ser. No. 11/267,555 filed Nov. 4, 2005; PCT application no. PCT/US05/39776, filed Nov. 4, 2005, 60/797,449, filed May 3, 2006 and 60/798,065, filed May 4, 2006, the disclosures of which are incorporated by reference; see also Chung et al., Nature, Oct. 16, 2005 (electronically published ahead of print) and Chung et al., Nature V. 439, pp. 216-219 (2006), the disclosures of each of which are incorporated by reference).

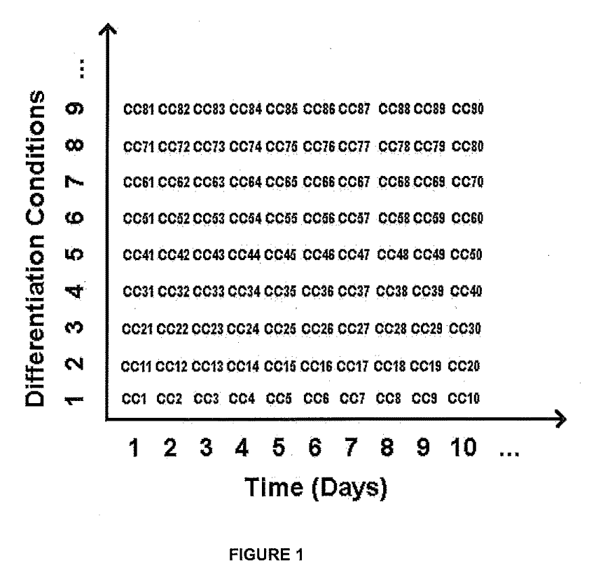

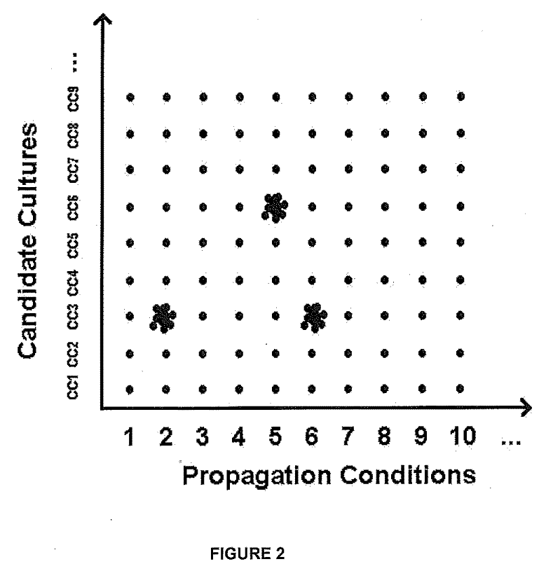

The present invention thus provides novel methods for the culture of mammalian pluripotent stem cell-derived cells from a single cell by first performing a differentiation step. In this differentiation step, pluripotent stem cells are differentiated under a variety or combination of different conditions leading to heterogeneous populations of cells herein referred to as candidate cultures (“CC”) (see FIG. 1). These candidate cultures may be identified, such as with bar coding, as candidate cultures (in the case of FIG. 1 as candidate cultures 1-90 (CC1-90)). In a second step (see FIG. 2), said candidate cultures are disaggregated so as to produce single cells that are separated such that when the cells from the candidate cultures are exposed to culture conditions that promote cellular proliferation or propagation, said single cells from the candidate culture may proliferate and expand in cell number in a manner allowing said proliferating cells to be later retrieved for use. To produce single cells, the cells may be plated at limiting dilution or at low density in cloning cylinders. To produce oligoclonal cells, the cells may be plated at a higher density such that clusters of related cells are isolated based on morphology or by sampling of the cluster and testing by PCR for markers of interest. Cells of interest may also be picked from among the cells plated at low density wherein clonal derivation is nearly certain. The conditions to promote differentiation in step (1) to generate candidate cultures and the conditions to promote propagation are chosen so as to make an array of combinations of conditions to screen for many possible candidate cultures and many possible propagation conditions.

The propagated single cell-derived cells of this invention have utility, for example, in research in cell biology, for the production of ligands for differentiation antigens, for the production of growth factors, for drug discovery, as feeder cells to obtain other such cells or as feeder cells for totipotent or pluripotent stem cells (such as hES cells), and for cell-based therapy and transplantation in human and veterinary medicine.

In one embodiment of the invention, the pluripotent stem cells are differentiated under a variety or combination of different conditions, such as those conditions listed, for example, in Table I. The differentiation conditions may include members of the EGF family of ligands; members of the EGF receptor/ErbB receptor family; members of the FGF ligand family; members of the FGF Receptor family; FGF regulators; Hedgehog family proteins; Hedgehog Regulators; members of the IGF family of ligands; IGF-I Receptor (CD221); members of the insulin growth factor-like binding protein (IGFBP) family of proteins; members of the Receptor Tyrosine Kinase family to sequester certain ligands; members of the proteoglycan family and proteoglycan regulators; members of the SCF, Flt-3 Ligand & M-CSF family; members of the Activin family; members of the BMP (Bone Morphogenetic Protein) family; members of the GDF (Growth Differentiation Factor) family; members of the GDNF Family of Ligands; members of the TGF-beta family of proteins; other TGF-beta Superfamily Ligands; members of the TGF-beta superfamily of receptors; modulators of the TGF-beta superfamily; members of the VEGF/PDGF family of factors; members of the family of Dickkopf proteins & Wnt inhibitors; members of the Frizzled family of factors and related proteins; members of the Wnt family of ligands; other Wnt-related Molecules; other factors known to influence the growth or differentiation of cells; members of the steroid family of hormones; members of the extracellular/membrane family of proteins; extracellular matrix proteins; ambient oxygen conditions; animal serum conditions; members of the interleukin family of proteins; members of the protease family of proteins; any one of the amino acids; members of the prostaglandin family; members of the retinoid receptor agonists/antagonists; a variety of different commercial cell culture media such as those listed in Table I; or miscellaneous inducers.

In another embodiment of the invention, the pluripotent stem cells are differentiated under a variety or combination of different conditions, such as any compounds or agents that belong to the family of teratogens listed, for example, but not limited to, those in Table IV. Tetratogens refer to any agents or compounds known to affect differentiation in vivo.

In certain embodiments of the invention, the various culture conditions that may be used in the first differentiation step or the subsequent propagation step include but are not limited to: plating the cells directly on a culture vessel wall, such as a dish, multiwell dish, flask, or roller bottle; attaching the cells to beads, microcarriers or disks, or solid or hollow fibers; encapsulating the cells in gels such as alginates; culturing the cells in semisolid media as is well known in the art for the culture of hematopoietic and other bone marrow-derived cells grown in suspension; culturing the cells in ovo, such as in juxtaposition with SPF chicken unfertilized eggs or fertilized SPF eggs in juxtaposition with avian embryonic cells; culturing the cells in microdrops, in hanging drops, as cell aggregates analogous to mammospheres and neurospheres; plating the cells on tissue culture substrates with added ECM components, incubating the cells to extracts in solution, in vesicles such as liposomes, or RNA extracts, including micro RNA extracts from differentiated cells such as, but not limited to, those listed in Table II, or differentiating cells such as, but not limited to, those listed in Table III; culturing the cells in various media including, but not limited to: defined media, media with animal sera, conditioned media with cells of defined cell types, including stromal cells, parenchymal cells, media conditioned with tissue, including embryonic and fetal anlagen or media conditioned in the heterogeneous culture from which the single cells were originally isolated, or conditioned medium obtained from the original culture of differentiated cells prior to trypsinization or such conditioned medium at 10% or 50% of the medium.

In another embodiment of the invention, the cells can be co-cultured with inducing cells on one layer, said inducing cells including stromal cells, parenchymal cells, embryonic and fetal anlagen or single cell-derived colonies on another layer.

In another embodiment of the invention, the single cell-derived or oligoclonal derived cells may be used as feeders or inducer cells for cell derivation of new cell types. The single cell or oligoclonal-derived feeder/inducer cell lines may be cultured in a variety of conditions and combined with a heterogenous mixture of candidate cells. The single cell or oligoclonal-derived feeder/inducer cells may also be mitotically inactivated using, for example, mitomycin C or ionizing radiation.

The complete media used in the isolation of single cell-derived cells may be defined medium without sera or other uncharacterized ingredient such as D-MEM/F-12 (1:1), and with insulin, transferrin, epidermal growth factor, leutinizing hormone or follicle stimulating hormone, somatomedin and growth hormone with HEPES buffer added to 15 mM to compensate for the loss of the buffering capacity of serum.

Conditions may be used to promote the growth of cells at clonal densities such as culturing the cells in an oxygen partial pressure less than that of the ambient atmosphere, such as 1-10% oxygen, preferably 3-5% oxygen, culturing the cells in media lacking phenol red, and/or culturing the cells with the addition of agents useful in metabolizing the toxic effects of oxygen such as the addition of 0.1 nM-10 μM selenium, preferably 1.0 nM-1 μM selenium, 10⁻⁵-10⁻⁷ M N-acetyl cysteine, (preferably 10⁻⁵M), and/or 500 U/mL of catalase.

In another embodiment of the invention, cells from the first differentiation step but prior to the clonal or oligoclonal propagation step, are placed in growth media similar to or identical to that in which they will be clonally or oligoclonally expanded in order to increase the number of cells capable of propagating in the medium of the second step. This enrichment step allows an increased number and more predictable number of cells to proliferate in the final clonal or oligoclonal medium of the second step. In some cases where the medium of the initial differentiation step is identical to or similar to the medium in which the cells will be clonally or oligoclonally expanded, the enrichment step may also increase the number of proliferating cells such that the heterogeneous mixture may be cryopreserved and in the event that the clonal or oligoclonal isolation yields useful cell types, the cryopreserved heterogeneous mixture of cells may be thawed and used as a source of cells for clonal or oligoclonal isolation again. Therefore, in one embodiment, the enrichment step is part of the initial differentiation step in that the culture medium of the first differentiation step is identical to, or similar to, that of the second clonal or oligoclonal propagation step. Alternatively, the enrichment step may be a separate step. The cells may be initially differentiated in one medium, then the heterogeneous mixture of cells can be transferred at normal cell culture densities to a different medium of the second clonal or oligoclonal expansion step. The cells are cultivated in that medium in a separate step. After a period of time of 2-30 days (preferably 5-14 days) that allows for the percentage of cells capable of being propagated in the medium to be increased, the heterogeneous mixture of cells is then clonally or oligoclonally expanded as described herein.

In another embodiment of the invention, the enrichment step may be effected or facilitated by physical separation of various subsets of the heterogeneous mixture of cells from the first differentiation step and/or the enrichment step. These subsets may, for example, represent cells of one or more lineages or at one or more stages of maturation or differentiation. One way to achieve this is to react the cells with a ligand or ligands such as, but not limited to, antibodies useful to positively select or purify specific cell types, or to delete the heterogeneous mixture of cells of specific cell types. A person of ordinary skill in the art can be guided in this effort by the gene expression profile of cells. This gene expression profile of the cells can yield useful information on the cell surface gene expression of antigens or other molecules such as differentiation or lineage markers for which antibodies or other ligands to such markers are available. For example, the isolation of RNA with subsequent gene expression analysis can yield a profile of the expression of transcripts related to cell surface antigens, and these can be useful in purifying the heterogeneous mixture of cells of step (1)(a) and (1)(b) using affinity methods known in the art to increase the frequency of cells of a desired type for subsequent clonal isolation in steps (2)(a) and (2)(b) or the direct use of the cells without clonal or oligoclonal isolation. Accordingly, such antigens and markers are useful in the identification and purification of cells made by the method of this invention as is understood by one skilled in the art.

In addition, where it is understood in the art that a desired cell type displays a particular cell surface antigen, those desired cell types can be obtained at an increased frequency using the methods of the present invention by first enriching a heterogeneous mixture containing the desired cells using ligands to said known cell surface antigens. Such separation techniques may include, without limitation, fluorescence activated cell sorting (FACS), immunomagnetic selection in a positive or negative (i.e., depletion) direction using paramagnetic or superparamagnetic beads or particles, or positive or negative immunoaffinity selection on bead or fiber matrix columns.

For FACS, these techniques can be done using the appropriate primary antibodies labeled directly or indirectly with any of a number of available fluorochromes with desired spectral properties, such as fluorescein or phycoerythrin. Indirect labeling can be achieved by interposing a fluorochrome labeled secondary, tertiary or higher order antibody specific for the immunoglobulin species, class or subclass of the primary or preceding antibody, or to a hapten-like tag on the primary or preceding antibody such as DNP, digoxin, FITC, or biotin, among many others known in the art. Alternatively, immunoglobulin-binding proteins such as protein A, G or L, or ligand-binding molecules such as avidin or streptavidin with affinity to biotin or like molecules can be employed in place of any secondary or higher order antibody. FACS instruments, primary and indirect secondary antibodies and related reagents for these purposes, and cell labeling and sorting protocols are well-known to those skilled in the art, such as Becton-Dickinson Immunocytometry Systemx (San Jose, Calif.), Pharmingen (San Diego, Calif.), and R&D Systems (Minneapolis, Minn.) and Southern Biotech (Birmingham, Ala.).

Similar labeling strategies can be employed using the primary antibody or antibodies directly or indirectly linked to magnetic particles or other matrix materials. Magnetic particles in a variety of configurations and modifications, along with antibodies and other accessory reagents, magnetic separators and matrix materials, and both specific and generic selection protocols that can be adapted for these purposes by those skilled in the art are available from numerous suppliers, such as MACS Microbeads from Miltenyi Biotec (Auburn, Calif.), DynaBeads from Invitrogen (Carlsbad, Calif.), MagCellect from R&D Systems (Minneapolis, Minn.), and RosetteSep from StemCell Technologies (Vancouver, BC, Canada). In addition, such CD antigens or other cell surface antigens can be employed in other direct or indirect labeling techniques similar to those described above to enrich said cell types from a mixture of cells by negatively selecting or depleting undesired cells using, without limitation, complement-mediated cell lysis. The cells to be depleted might be distinguished, for example, by one or more antigens associated with certain lineages or stage(s) of differentiation. In this technique, the undesired cells in the cell mixture are labeled directly or indirectly with antibodies that are able to activate or fix complement, and then incubated briefly (usually an hour or less) with a source of active complement at or near physiological temperature (e.g., 37 C) during which time these cells undergo lysis. A commonly used source of such complement, among others known to those in the art, is non-heat-inactivated newborn rabbit serum, available for example from Invitrogen (Carlsbad, Calif.).

In another embodiment of the invention, the first differentiation step may be mediated by siRNA or other similar techniques (i.e. ribozymes, antisense). The use of siRNA (including miRNAs that naturally regulate cell differentiation and are known in the art) in the first differentiation step may provide a means of steering the differentiation of the pluripotent stem cells to make a heterogeneous population of cells that are biased in some direction, for example, to become endoderm, mesoderm or ectoderm. For example, transfection of embryonic stem cells with OCT4- or Nanog-targeted RNAi is sufficient to induce differentiation towards extraembryonic lineages (Hough et al. Stem Cells. 2006 Feb. 2; Epub). RNAi has been shown to work in a number of cells, including mammalian cells, such as ES cells.

In another embodiment of the invention, the initial pluripotent stem cells may express the catalytic component of telomerase reverse transcriptase (hTERT) (such as when the cells are ES cell lines) and telomere length may be maintained in cultures of said stem cells such that differentiated derived cells made according to the present invention have relatively long proliferative lifespans allowing for clonal, even up to five serial clonal isolations. In addition, since the cells express TERT, telomere length may be increased through the addition of agents to the culture that increase mean telomere length in said cells. Telomerase activity is repressed when said cells undergo differentiation, but the derived cells are able to retain an increased proliferative lifespan when compared to normal somatic cells of that species. The increase in mean telomere length in the TERT-expressing pluripotent stem cells, such as ES cells, leads to an increased proliferative lifespan of the telomerase-negative derived cells.

Pluripotent stem cells that are naturally expressing the catalytic component of telomerase reverse transcriptase (hTERT) and normally repress that expression when the pluripotent stem cells differentiate may be treated with exogenous agents to increase the mean telomere length in the pluripotent stem cells. The differentiated cells from said stein cells will display an increased replicative lifespan when compared to their normal counterparts. Such agents may include, but are not limited to, inhibitors of DNA cytosine-C5-methyltransferase 3 beta (DNMT3B; accession number NM_{—}175849.1) using, for example, siRNA constructs targeting the mRNA transcripts of that gene, or small molecule inhibitors of the enzyme. The knockout of DNA3B in tumor cells has been reported to increase the mean telomere length in those cells, but the inhibition of that enzyme would not necessarily be expected in any normal cell type such as pluripotent stem cells with germ-line telomere length. Additional molecular targets to transiently increase mean telomere length include, for example, modulators of poly (ADP-ribose) polymerase (ADPRT; accession number NM_{—}001618.2), TERF1, TERF2, and the exogenous addition of estrogen or telomeric oligonucleotides.

In certain embodiments of the invention, the pluripotent stem cells may be transfected with a DNA construct such that hTERT or the TERT gene of another species is constitutively or inducibly activated by an extrinsic activator as is well known in the art. In some embodiments, the TERT gene may be derived from mammalian species other than human, including, but not limited to, equine, canine, porcine, bovine, and ovine sources; rodent sources such as mouse or rat; or avian sources. The differentiated cell clones generated according to the present invention may then be constitutively immortal or conditionally immortal. Such cells will be useful where the expansion of said cells would normally erode telomere length below a desired level.

In another embodiment of the invention, the first differentiation step may be mediated by reprogramming the expression profile of a cell to convert it into that of a desired cell type. For example, the pluripotent stem cells can be reprogrammed by incubating the nucleus or chromatin mass from said pluripotent stem cells with a reprogramming media (e.g., a cell extract) under conditions that allow nuclear or cytoplasmic components such as transcription factors to be added to, or removed from, the nucleus or chromatin mass (see U.S. application Ser. No. 10/910,156, filed Aug. 2, 2004 (US publication no. 20050014258, published Jan. 20, 2005); see also U.S. application No. 60/705,625, filed Aug. 3, 2005; U.S. application No. 60/729,173, filed Oct. 20, 2005; U.S. application No. 60/818,813, filed Jul. 5, 2006). The added transcription factors may promote the expression of mRNA or protein molecules found in cells of the desired cell type, and the removal of transcription factors that would otherwise promote expression of mRNA or protein molecules found in said pluripotent stem cells. If desired, the chromatin mass may then be incubated in an interphase reprogramming media (e.g., an interphase cell extract) to reform a nucleus that incorporates desired factors from either reprogramming media. The nucleus or chromatin mass is then inserted into a recipient cell or cytoplast, forming a reprogrammed cell of the desired cell type. In another embodiment, a permeabilized cell is incubated with a reprogramming media (e.g., a cell extract) to allow the addition or removal of factors from the cell, and then the plasma membrane of the permeabilized cell is resealed to enclose the desired factors and restore the membrane integrity of the cell. If desired, the steps of any of these methods may be repeated one or more times or different reprogramming methods may be performed sequentially to increase the extent of reprogramming, resulting in a greater alteration of the mRNA and protein expression profile in the reprogrammed cell. Furthermore, reprogramming medias may be made representing combinations of cell functions (e.g., medias containing extracts or factors from multiple cell types) to produce unique reprogrammed cells possessing characteristics of multiple cell types.

Although human cells are preferred for use in the invention, the cells to be used in the method of the invention are not limited to cells from human sources. Cells from other mammalian species including, but not limited to, equine, canine, porcine, bovine, and ovine sources; or rodent species such as mouse or rat; or cells from other species such as avian, in particular SPF chicken ES-derived or embryo-derived cells, may be used.

In addition, cells that are spontaneously, chemically or virally transfected or recombinant cells or genetically engineered cells may also be used in this invention. For those embodiments that incorporate more than one cell type, chimeric mixtures of normal cells from two or more sources; mixtures of normal and genetically modified or transfected cells; or mixtures of cells of two or more species or tissue sources may be used.

In addition, clonal or oligoclonal cells isolated according to the invention may be modified to artificially inhibit cell cycle inhibitory factors or otherwise stimulate the cells to replicate rapidly through means well known in the art. Said artificial stimulation of the cell cycle may be made reversible through means well known in the art, including but not limited to, the use of inducible promoters, temperature sensitive promoters, RNAi, transient delivery of proteins into the cells, or by other means known in the art. Any method known in the art to overcome cell cycle inhibition may be used with the invention. By way of nonlimiting example, the retinoblastoma and p53 pathways may be inhibited, such as by the use of T-antigen, the adenovirus proteins E1A and E1B, or the papillomavirus proteins E6 and E7 or the cell cycle can be induced by other means such as by the up-regulation of CDK4 as is known in the art to override p16 cell cycle checkpoint. In certain embodiments, protein agents may be modified with protein transduction domains as described herein. By way of nonlimiting example, pluripotent stem cells such as ES, EG, EC or ED cells may be transfected with a construct that leads to an inducible SV40 T-antigen or CDK4 such as a temperature sensitive T-antigen or CDK4. As a result, cells can be allowed to differentiate into an initial heterogeneity of cell types and then clonally or oligoclonally expanded under conditions wherein the SV40 T-antigen or CDK4 genes are induced to stimulate the proliferation of the cells. When sufficient numbers of cells are obtained, the expression of SV40 T-antigen or CDK4 may be downregulated by reversing the steps that led to the activation of the gene, or by the physical removal of the gene or genes using recombinase technology as is well known in the art, such as through the use of the CRE recombinase system or the use of FLP recombinase.

In certain embodiments, SV40 T-antigen or CDK4 may be added during the first differentiation step or at the beginning of the clonal or oligoclonal expansion/propagation step. In certain embodiments, the import of SV40 T-antigen or CDK4 may be improved by delivery with liposomes, electroporation, or by permeabilization (see U.S. Patent Application No. 20050014258, herein incorporated by reference). For example, cells may be permeabilized using any standard procedure, such as permeabilization with digitonin or Streptolysin O. Briefly, cells are harvested using standard procedures and washed with PBS. For digitonin permeabilization, cells are resuspended in culture medium containing digitonin at a concentration of approximately 0.001-0.1% and incubated on ice for 10 minutes. For permeabilization with Streptolysin O, cells are incubated in Streptolysin O solution (see, for example, Maghazachi et al., 1997) for 15-30 minutes at room temperature. After either incubation, the cells are washed by centrifugation at 400×g for 10 minutes. This washing step is repeated twice by resuspension and sedimentation in PBS. Cells are kept in PBS at room temperature until use. Alternatively, the cells can be permeabilized while placed on coverslips to minimize the handling of the cells and to eliminate the centrifugation of the cells, thereby maximizing the viability of the cells.

Delivery of T-antigen or other proteins may be accomplished indirectly by transfecting transcriptionally active DNA into living cells (such as the cells of this invention) where the gene is expressed and the protein is made by cellular machinery. Several methods are known to one of skill in the art to effectively transfect plasmid DNA including calcium phosphate coprecipitation, DEAE dextran facilitated transfection, electroporation, microinjection, cationic liposomes and retroviruses. Any method known in the art may be used with this invention to deliver T-antigen or other proteins into cells.

In certain embodiments, protein is delivered directly into cells of this invention, thereby bypassing the DNA transfection step. Several methods are known to one of skill in the art to effectively deliver proteins into cells including microinjection, electroporation, the construction of viral fusion proteins, and the use of cationic lipids.

Electroporation may be used to introduce foreign DNA into mammalian (Neumann, E. et al. (1982) EMBO J. 1, 841-845), plant and bacterial cells, and may also be used to introduce proteins (Marrero, M. B. et al. (1995) J. Biol. Chem. 270, 15734-15738; Nolkrantz, K. et al. (2002) Anal. Chem. 74, 4300-4305; Rui, M. et al. (2002) Life Sci. 71, 1771-1778). Cells (such as the cells of this invention) suspended in a buffered solution of the purified protein of interest are placed in a pulsed electrical field. Briefly, high-voltage electric pulses result in the formation of small (nanometer-sized) pores in the cell membrane. Proteins enter the cell via these small pores or during the process of membrane reorganization as the pores close and the cell returns to its normal state. The efficiency of delivery is dependent upon the strength of the applied electrical field, the length of the pulses, temperature and the composition of the buffered medium. Electroporation is successful with a variety of cell types, even some cell lines that are resistant to other delivery methods, although the overall efficiency is often quite low. Some cell lines remain refractory even to electroporation unless partially activated.

Microinjection was first used to introduce femtoliter volumes of DNA directly into the nucleus of a cell (Capecchi, M. R. (1980) Cell 22, 470-488) where it can be integrated directly into the host cell genome, thus creating an established cell line bearing the sequence of interest. Proteins such as antibodies (Abarzua, P. et al. (1995) Cancer Res. 55, 3490-3494; Theiss, C. and Meller, K. (2002) Exp. Cell Res. 281, 197-204) and mutant proteins (Naryanan, A. et al. (2003) J. Cell Sci. 116, 177-186) can also be directly delivered into cells via microinjection to determine their effects on cellular processes first band. Microinjection has the advantage of introducing macromolecules directly into the cell, thereby bypassing exposure to potentially undesirable cellular compartments such as low-pH endosomes. All of these techniques can be used on the cells of this invention or the parent pluripotent cells.

Several proteins and small peptides have the ability to transduce or travel through biological membranes independent of classical receptor- or endocytosis-mediated pathways. Examples of these proteins include the HIV-1 TAT protein, the herpes simplex virus 1 (HSV-1) DNA-binding protein VP22, and the Drosophila Antennapedia (Antp) homeotic transcription factor. The small protein transduction domains (PTDs) from these proteins can be fused to other macromolecules, peptides or proteins to successfully transport them into a cell (Schwarze, S. R. et al. (2000) Trends Cell Biol. 10, 290-295). Sequence alignments of the transduction domains from these proteins show a high basic amino acid content (Lys and Arg) which may facilitate interaction of these regions with negatively charged lipids in the membrane. Secondary structure analyses show no consistent structure between all three domains. The advantages of using fusions of these transduction domains is that protein entry is rapid, concentration-dependent and appears to work with difficult cell types (Fenton, M. et al. (1998) J. Immunol. Methods 212, 41-48.). All of these techniques can be used on the cells of this invention or the parent pluripotent cells.

Liposomes have been rigorously investigated as vehicles to deliver oligonucleotides, DNA (gene) constructs and small drug molecules into cells (Zabner, J. et al. (1995) J. Biol. Chem. 270, 18997-19007; Felgner, P. L. et al. (1987) Proc. Natl. Acad. Sci. USA 84, 7413-7417). Certain lipids, when placed in an aqueous solution and sonicated, form closed vesicles consisting of a circularized lipid bilayer surrounding an aqueous compartment. These vesicles or liposomes can be formed in a solution containing the molecule to be delivered. In addition to encapsulating DNA in an aqueous solution, cationic liposomes can spontaneously and efficiently form complexes with DNA, with the positively charged head groups on the lipids interacting with the negatively charged backbone of the DNA. The exact composition and/or mixture of cationic lipids used can be altered, depending upon the macromolecule of interest and the cell type used (Felgner, J. H. et al. (1994) J. Biol. Chem. 269, 2550-2561). The cationic liposome strategy has also been applied successfully to protein delivery (Zelphati, O. et al. (2001) J. Biol. Chem. 276, 35103-35110). Because proteins are more heterogeneous than DNA, the physical characteristics of the protein such as its charge and hydrophobicity will influence the extent of its interaction with the cationic lipids. All of these techniques can be used on the cells of this invention or the parent pluripotent cells.

In certain embodiments Pro-Ject Protein Transfection Reagent may be used. Pro-Ject Protein Transfection Reagent utilizes a unique cationic lipid formulation that is noncytotoxic and is capable of delivering a variety of proteins into numerous cell types. The protein being studied is mixed with the liposome reagent and is overlayed onto cultured cells. The liposome:protein complex fuses with the cell membrane or is internalized via an endosome. The protein or macromolecule of interest is released from the complex into the cytoplasm free of lipids (Zelphati, O. and Szoka, Jr., F. C. (1996) Proc. Natl. Acad. Sci. USA 93, 11493-11498) and escaping lysosomal degradation. The noncovalent nature of these complexes is a major advantage of the liposome strategy as the delivered protein is not modified and therefore is less likely to lose its activity. All of these techniques can be used on the cells of this invention or the parent pluripotent cells.

In certain embodiments, the nuclear localization sequence of SV40 T-antigen may be modified. Protein transduction domains (PTD), covalently or non-covalently linked to T-antigen, allow the translocation of T-antigen across the cell membranes so the protein may ultimately reach the nuclear compartments of the cells. PTDs that may be fused with a Tag protein include the PTD of the HIV transactivating protein (TAT) (Tat 47-57) (Schwarze and Dowdy (2000) Trends Pharmacol. Sci. 21: 45-48; Krosl et al. (2003) Nature Medicine 9:1428-1432). For the HIV TAT protein, the amino acid sequence conferring membrane translocation activity 5 corresponds to residues 47-57 (YGRKKRRQRRR) (Ho et al. (2001) Cancer Research 61: 473-477; Vives et al. (1997) J. Biol Chem. 272: 16010-16017). This sequence alone can confer protein translocation activity. The TAT PTD may also be the nine amino acids peptide sequence RKKRRQRRR (Pauk et al. Mol Cells (2002) 30:202-8). The TAT PTD sequences may be any of the peptide sequences disclosed in Ho et al. (2001) Cancer Research 61: 473-477, including YARKARRQARR, YARZLAARQARA, YARAARRAARR, and RARAARRAARA. Other proteins that contain PTDs that may be fused with Tag include the herpes simplex virus 1 (HSV-1) DNA-binding protein VP22 and the Drosophila Antennapedia (Antp) transcription factor (Schwarze et al. (2000) Trends Cell Biol 10:290-295). For Antp, amino acids 43-58 (RQIKIWFQNRRMKWM) represent the protein transduction domain, and for HSV VP22 the PTD is represented by the residues DAATATRGRSAASRPTERPRAPARSASRPRRPVE. Alternatively, HeptaARG (RRRRRRR) or artificial peptides that confer transduction activity may be used as a PTD. The PTD may be a PTD peptide that is duplicated or multimerized; including one or more of the TAT PTD peptide YARAAARQARA, or a multimer consisting of three of the TAT PTD peptide YARARARQARA. Techniques for making fusion genes encoding fusion proteins are well known in the art. The joining of various DNA fragments coding for different polypeptide sequences may be performed in accordance with conventional techniques. The fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed to generate a chimeric gene sequence (see, for example, Current Protocols in Molecular Biology, eds. Ausubel et al., John Wiley & 20 Sons: 1992). A fusion gene coding for a purification leader sequence, such as a poly-(His) sequence, may be linked to the N-terminus or C-terminus of the desired portion of the Tag polypeptide or Tag-fusion protein allowing the fusion protein be purified by affinity chromatography using a metal resin. The purification leader sequence can then be subsequently removed by treatment with enterokinase to provide the purified Tag polypeptide (e.g., see Hochuli, E., et al (1987) J. Chromatog. 411:177-184). T antigen that is provided in the media may be excreted by another cell type. The other cell type may be a feeder layer, such as a mouse stromal cell layer transduced to express secretable T antigen. For example, T antigen may be fused to or engineered to comprise a signal peptide, or a hydrophobic sequence that facilitates export and secretion of the protein. Alternatively, T antigen, as a fusion protein covalently or linked to a PTD or as a protein or a fusion protein non-covalently linked to a PTD, may be added directly to the media. In certain embodiments, cell lines are created that secrete the TAT-T antigen fusion protein (see Derer, W. et al. (2001) The FASEB Journal, Published online). Conditioned medium from TAT-T antigen secreting cell lines is subsequently added to recipient cell lines to promote cell growth.

Human embryo-derived (hED) cells are cells that are derived from human embryos such as human preimplantation embryos, postimplantation embryos (such as aborted embryonic tissue) or pluripotent cell lines such as ES cell lines derived from human preimplantation embryos. Human zygotes, 2 or more cell premorula stage such as blastomeres, morula stage, compacting morula, blastocyst embryo inner cell masses, or cells from developing embryos all contain pluripotent cells. Such cells may be differentiated using techniques described herein to yield the initial heterogeneous population of cells of the first step. Because such culture conditions may induce the direct differentiation of the cells without allowing the propagation of a hES cell line, the probability of a hES cell contaminating the resulting clonal or oligoclonal cultures is reduced.

The single cells of this invention (made by the methods of this invention) may be used as the starting point for deriving various differentiated cell types. The single cells of this invention may be the precursors of any cell or tissue lineage.

In another embodiment of the invention, the clonal or oligoclonal populations may be derived from embryonic tissues. For example, embryonic tissue may be dissected and the cells disaggregated. Such disaggregated cells may then be used as the starting parent pluripotent cells of the methods of this invention.

There have been numerous attempts in the prior art to differentiate embryonic stem cells, embryonal carcinoma cells, and embryonic germ cells into various cell types. These methods have been only marginally successful due to problems with culturing and characterizing the complex mixture of cell types originating out of differentiating ES, EC, and EG cultures in vitro. It has not been possible to preserve a pure culture of the differentiated cell type without having the culture overgrown with fibroblastic or other contaminating cell types. See, Ian Freshney, Culture of Animal Cells: A Manual of Basic Technique (5th Ed.), New York: Wiley Publishing, 2005, p. 217. The methods of the present application can overcome those difficulties due in part to the unexpected clonogenicity of ES, EC, EG, and ED-derived cells. In addition, while ES cell lines such as human ES cell lines originate from cultures of ICM cells, it is not therefore obvious that observations made with ES cell lines apply to ED cells, especially those made by direct differentiation from the embryo without the generation of an ES cell line. For example, while the ICM of the preimplantation embryo contains totipotential cells capable of differentiating into all somatic cell lineages and the germ-line, many efforts have been made in the past to generate ES cell lines that retain the totipotency of the ICM and can still contribute to the germ-line. Such ES cell lines would therefore, like mouse ES cells, be useful in introducing heritable genetic modifications into animals. Nevertheless, other than mouse ES cells, mammalian cultured ICM cells generally lose the ability to contribute to the germ-line when introduced into the blastocyst and are therefore not equivalent to the ICM. Therefore, it would not be obvious to one skilled in the art that ED cells cultured without the generation of an ES cell line would differentiate or propagate in the same manner as ES cells. However, in the present invention, it is disclosed that totipotential cells of preimplantation embryos, including zygotes, blastomeres, cells from the morula staged embryo, cells from the inner cell mass, and cells from the embryonic disc are in fact equivalent to ES cell lines and can simply be substituted for ES cell in the present invention.

In one embodiment of the application, any methods of differentiating, propagating, identifying, isolating, or using stem cells known in the art (for example, U.S. Pat. Nos. 6,953,799, 7,029,915, 7,101,546, 7,129,034, 6,887,706, 7,033,831, 6,989,271, 7,132,286, 7,132,287, 6,844,312, 6,841,386, 6,565,843, 6,908,732, 6,902,881, 6,602,680, 6,719,970, 7,112,437, 6,897,061, 6,506,574, 6,458,589, 6,774,120, 6,673,606, 6,602,711, 6,770,478, 6,610,535, 7,045,353, 6,903,073, 6,613,568, 6,878,543, 6,670,397, 6,555,374, 6,261,841, 6,815,203, 6,967,019, 7,022,666, 6,423,681, 6,638,765, 7,041,507, 6,949,380, 6,087,168, 6,919,209, 6,676,655, 6,761,887, 6,548,299, 6,280,718, 6,656,708, 6,255,112, 6,413,773, 6,225,119, 6,056,777, 6,962,698, 6,936,254, 6,942,995, 6,924,142, 6,165,783, 6,093,531, 6,379,953, 6,022,540, 6,586,243, 6,093,557, 5,968,546, 6,562,619, 5,914,121, 6,251,665, 6,228,640, 5,948,623, 5,766,944, 6,783,775, 6,372,262, 6,147,052, 5,928,945, 6,096,540, 6,709,864, 6,322,784, 5,827,740, 6,040,180, 6,613,565, 5,908,784, 5,854,292, 6,790,826, 5,677,139, 5,942,225, 5,736,396, 5,648,248, 5,610,056, 5,695,995, 6,248,791, 6,051,415, 5,939,529, 5,922,572, 6,610,656, 6,607,913, 5,844,079, 6,686,198, 6,033,906, 6,340,668, 6,020,197, 5,766,948, 5,369,030, 6,001,654, 5,955,357, 5,700,691, 5,498,698, 5,733,878, 5,384,331, 5,981,165, 6,464,983, 6,531,445, 5,849,686, 5,197,985, 5,246,699, 6,177,402, 5,488,040, 6,667,034, 5,635,386, 5,126,325, 5,994,518, 5,032,507, 5,847,078, 6,004,548, 5,529,982, 4,342,828, 7,105,344, 7,078,230, 7,074,911, 7,053,187, 7,041,438, 7,030,292, 7,015,037, 7,011,828, 6,995,011, 6,969,608, 6,967,102, 6,960,444, 6,929,948, 6,878,542, 6,867,035, 6,866,843, 6,833,269, 6,828,144, 6,818,210, 6,800,480, 6,787,355, 6,777,231, 6,777,230, 6,749,847, 6,737,054, 6,706,867, 6,677,306, 6,667,391, 6,642,048, 6,638,501, 6,607,720, 6,576,464, 6,555,318, 6,545,199, 6,534,052, RE37,978, 6,461,865, 6,432,711, 6,399,300, 6,372,958, 6,369,294, 6,342,356, 6,337,184, 6,331,406, 6,271,436, 6,245,566, 6,235,970, 6,235,969, 6,215,041, 6,204,364, 6,194,635, 6,171,824, 6,090,622, 6,015,671, 5,955,290, 5,945,577, 5,914,268, 5,874,301, 5,866,759, 5,865,744, 5,843,422, 5,830,510, 5,795,569, 5,766,581, 5,733,727, 5,725,851, 5,712,156, 5,688,692, 5,656,479, 5,602,301, 5,370,870, 5,366,888, and 5,332,672, and U.S. patent publication nos. 20060251642, 20060217301, 20060216820, 20060193769, 20060161996, 20060134784, 20060134782, 20060110828, 20060104961, 20060088890, 20060079488, 20060078989, 20060068496, 20060062769, 20060024280, 20060015961, 20060009433, 20050244969, 20050244386, 20050233447, 20050221483, 20050164377, 20050153425, 20050149998, 20050142102, 20050130147, 20050118228, 20050106211, 20050054102, 20050032207, 20040260079, 20040228899, 20040193274, 20040152189, 20040151701, 20040141946, 20040121464, 20040110287, 20040052768, 20040028660, 20040028655, 20040018178, 20040009595, 20030203003, 20030175680, 20030161819, 20030148510, 20030082155, 20030040111, 20030040023, 20030036799, 20030032187, 20030032183, 20030031657, 20020197240, 20020164307, 20020098584, 20020098582, 20020090714, 20020022259, 20020019018, 20010046489, 20010024824, and 20010016203) are used in combination with the methods of the present application in differentiating, propagating, identifying, isolating, or using directly differentiated embryo-derived cells (i.e., substituting ED cells for ES cells and directly differentiating the ED cells). In certain embodiments, only the initial differentiation procedure from the prior art is used in combination with the present methods. In certain embodiments, ED cells are directly differentiated in the manner disclosed in the art for ES cells, and following differentiation, cells are plated resulting in isolating a number of individual cultures of cells or a number of individual cultures of cells that are oligoclonal, wherein one or more of said cultures comprise cells with reduced differentiation potential than the starting pluripotent stem cells and wherein each of said individual cultures having only one cell may be propagated into a pure clonal culture of cells and wherein each of said individual cultures of cells having cells that are oligoclonal may be propagated into a larger number of cells, and one or more (or all) of said individual cultures of cells is propagated. To summarize, ED cells are differentiated in step 1 of this invention according to the methods in the art and then the heterogenous population of cells so generated are cultured and propagated according to step 2 of this invention.

In another aspect of the invention, the methods of this invention result in the derivation of endodermal cells from a single cell differentiated or in the process of differentiating from pluripotent stem cells such as, but not limited to, hES, hEG, hiPS, hEC or hED cells.

In another aspect of the invention, the methods of this invention result in the derivation of mesodermal cells from a single cell differentiated or in the process of differentiating from pluripotent stem cells such as, but not limited to, hES, hEG, hiPS, hEC or hED cells.

In another aspect of the invention, the methods of this invention result in the derivation of ectodermal cells from a single cell differentiated or in the process of differentiating from pluripotent stem cells such as, but not limited to, hES, hEG, hiPS, hEC or hED cells.

In another aspect of the invention, the methods of this invention result in the derivation of neuroglial precursor cells from a single cell differentiated or in the process of differentiating from pluripotent stem cells such as, but not limited to, hES, hEG, hiPS, hEC or hED cells.

In another aspect of the invention, the methods of this invention result in the derivation of hepatic cells or hepatic precursor cells from a single cell differentiated or in the process of differentiating from pluripotent stem cells such as, but not limited to, hES, hEG, hiPS, hEC or hED cells.

In another aspect of the invention, the methods of this invention result in the derivation of chondrocyte or chondrocyte precursor cells from a single cell differentiated or in the process of differentiating from pluripotent stem cells such as, but not limited to, hES, hEG, hiPS, hEC or hED cells.

In another aspect of the invention, the methods of this invention result in the derivation of myocardial or myocardial precursor cells from a single cell differentiated or in the process of differentiating from pluripotent stem cells such as, but not limited to, hES, hEG, hiPS, hEC or hED cells. Such myocardial precursor cells may also be produced by direct differentiation as described herein. An example of the production of myocardial precursors from hES cells is described in Example 31 and production from hED cells is shown in Example 38.

In another aspect of the invention, the methods of this invention result in the derivation of gingival fibroblast or gingival fibroblast precursor cells from a single cell differentiated or in the process of differentiating from pluripotent stem cells such as, but not limited to, hES, hEG, hiPS, hEC or hED cells.

In another aspect of the invention, the methods of this invention result in the derivation of pancreatic beta cells or pancreatic beta precursor cells from a single cell differentiated or in the process of differentiating from pluripotent stem cells such as, but not limited to, hES, hEG, hiPS, hEC or hED cells.

In another aspect of the invention, the methods of this invention result in the derivation of retinal precursor cells with from a single cell differentiated or in the process of differentiating from pluripotent stem cells such as, but not limited to, hES, hEG, hiPS, hEC or hED cells.

In another aspect of the invention, the methods of this invention result in the derivation of hemangioblasts from a single cell differentiated or in the process of differentiating from pluripotent stein cells such as, but not limited to, hES, hEG, hiPS, hEC or hED cells.

In another aspect of the invention, the methods of this invention result in the derivation of dermal fibroblasts with prenatal patterns of gene expression from a single cell differentiated or in the process of differentiating from pluripotent stem cells such as, but not limited to, hES, hEG, hiPS, hEC or hED cells.

Dermal fibroblasts derived according to the invention can be grown on a biocompatible substratum and engrafted on the neodermis of artificial skin covering a wound. Autologous keratinocytes may also be cultivated on a commercially available membrane such as Laserskin™ using the methods provided in this invention.

In another embodiment of the present invention, it is possible to simplify burn treatment further and to save lives of patients having extensive burns where sufficient autologous skin grafts cannot be repeatedly harvested in a short period of time. The dead skin tissue of a patient with extensive burns can be excised within about three to seven days after injury. The wound can be covered with any artificial skin, for example Integra™, or any dermal equivalent thereof, and dermal keratinocytes or dermal fibroblasts produced according to the methods of this invention or derived from said cells may thereafter be engrafted on the neodermis of the artificial skin, with resultant lower rejection and infection incidences.

Epidermolysis bullosa (“EB”) is a group of heritable diseases that result in a loss of mechanical strength in the skin, in particular, separation of the epidermis from the dermis (blistering). EB patients have fragile skin which can blister even from mild, such as skin-to-skin, contact. These patients suffer from constant pain and scarring, which, in the worse forms, leads to eventual disfigurement, disability and often early death. EB patients lack anchors that hold the layers of their skin together and as a consequence, any activity that rubs or causes pressure produces a painful sore that has been compared to a second-degree burn. One of the forms of EB is lethal in the first weeks or months of life. Some are more long-term and cause pain and mutilation throughout the patient's lifetime. Infection is a serious, ongoing concern and no treatment for EB has been effective. To date, parents' only hope has been to attempt to protect the child's skin with gauze and ointments, to prevent and protect the wounds and healthy skin. The manifestation of the disease is highly variable depending on the locus of the mutation. Traditionally, there are three categories: the simplex form with separation within the keratinocytes, the junctional forms with separation the lamina lucida of the basement membrane, and the dystrophic forms with separation in the papillary dermis. There is now evidence of another variant at the level of hemidesmosomes and the basal cell/lamina lucida interface (Uitto et al., Am J Med Genet C Semin Med Genet 131C:61-74 (2004)). Accordingly, dermal keratinocytes or dermal fibroblasts produced according to the methods of this invention or derived from said cells may be engrafted onto wound sites of EB patients to lower the incidence of infection and prevent further blistering.

The cells produced according to the methods of this invention or derived from said cells may also be combined with biological or synthetic matrices as is well known in the art. For example, dermal fibroblasts may be combined with collagen, including collagen that has been cross-linked by chemical or physical methods, and/or with other extracellular matrix components such as fibronectin, fibrin, proteoglycans, among others. The cells may be used in combination with hyaluronan (HA).

Some embodiments of the invention provide a matrix for implantation into a patient. In some embodiments, the matrix is seeded with a population of keratinocytes or dermal fibroblast cells derived according to methods of this invention. The matrix may contain or be pre-treated with one or more bioactive factors including, for example, drugs, anti-inflammatory agents, antiapoptotic agents, and growth factors. The seeded or pre-treated matrices can be introduced into a patient's body in any way known in the art, including but not limited to, implantation, injection, surgical attachment, transplantation with other tissue, injection, and the like. The matrices of the invention may be configured to the shape and/or size of a tissue or organ in vivo. The scaffolds of the invention may be flat or tubular or may comprise sections thereof. The scaffolds of the invention may also be multilayered.

To form a bilayer tissue construct comprising a cell-matrix construct and a second cell layer thereon, the method of this invention additionally comprises the step of: culturing cells of a second type on a surface of the formed tissue-construct to produce a bilayered or multilayered tissue construct.

An extracellular matrix-producing cell type for use in the invention may be any cell type capable of producing and secreting extracellular matrix components and organizing the extracellular matrix components to form a cell-matrix construct. More than one extracellular matrix-producing cell type may be cultured to form a cell-matrix construct. Cells of different cell types or tissue origins may be cultured together as a mixture to produce complementary components and structures similar to those found in native tissues. For example, the extracellular matrix-producing cell type may have other cell types mixed with it to produce an amount of extracellular matrix that is not normally produced by the first cell type. Alternatively, the extracellular matrix-producing cell type may also be mixed with other cell types that form specialized tissue structures in the tissue but do not substantially contribute to the overall formation of the matrix aspect of the cell-matrix construct, such as in certain skin constructs of the invention. All cells are either produced by methods of this invention or derived from said cells.

While any extracellular matrix-producing cell type may be used in accordance with this invention, the preferred cell types for use in this invention are derived from mesenchyme. More preferred cell types are fibroblasts, stromal cells, and other supporting connective tissue cells, most preferably human dermal fibroblasts found in human dermis for the production of a human dermal construct. Fibroblast cells, generally, produce a number of extracellular matrix proteins, primarily collagen. There are several types of collagens produced by fibroblasts, however, type I collagen is the most prevalent in vivo. Human fibroblast cell strains can be derived from a number of sources, including, but not limited to, neonate male foreskin, dermis, tendon, lung, umbilical cords, cartilage, urethra, corneal stroma, oral mucosa, and intestine. The human cells may include, but need not be limited to, fibroblasts, but may include: smooth muscle cells, chondrocytes and other connective tissue cells of mesenchymal origin. It is preferred, but not required, that the origin of the matrix-producing cell used in the production of a tissue construct be derived from a tissue type that it is to resemble or mimic after employing the culturing methods of the invention. For instance, in the embodiment where a skin-construct is produced, the preferred matrix-producing cell is a fibroblast, preferably of dermal origin. In another preferred embodiment, fibroblasts isolated by microdissection from the dermal papilla of hair follicles can be used to produce the matrix alone or in association with other fibroblasts. In the embodiment where a corneal-construct is produced, the matrix-producing cell is derived from corneal stroma. Cell donors may vary in development and age. Cells may be derived from donor tissues of embryos, neonates, or older individuals including adults. Embryonic progenitor cells such as mesenchymal stem cells may be used in the invention and induced to differentiate to develop into the desired tissue. All cells are either produced by methods of this invention or derived from said cells.

Recombinant or genetically-engineered cells may be used in the production of the cell-matrix construct to create a tissue construct that acts as a drug delivery graft for a patient needing increased levels of natural cell products or treatment with a therapeutic. The cells may produce and deliver to the patient via the graft recombinant cell products, growth factors, hormones, peptides or proteins for a continuous amount of time or as needed when biologically, chemically, or thermally signaled due to the conditions present in the patient. Either long or short-term gene product expression is desirable, depending on the use indication of the cultured tissue construct. Long term expression is desirable when the cultured tissue construct is implanted to deliver therapeutic products to a patient for an extended period of time. Conversely, short term expression is desired in instances where the cultured tissue construct is grafted to a patient having a wound where the cells of the cultured tissue construct are to promote normal or near-normal healing or to reduce scarification of the wound site. Once the wound has healed, the gene products from the cultured tissue construct are no longer needed or may no longer be desired at the site. Cells may also be genetically engineered to express proteins or different types of extracellular matrix components which are either “normal” but expressed at high levels or modified in some way to make a graft device comprising extracellular matrix and living cells that is therapeutically advantageous for improved wound healing, facilitated or directed neovascularization, or minimized scar or keloid formation. These procedures are generally known in the art, and are described in Sambrook et al., Molecular Cloning, A Laboratory Manual, Cold Spring Harbor Press, Cold Spring Harbor, N.Y. (1989), incorporated herein by reference. All of the above-mentioned types of cells are included within the definition of a “matrix-producing cell” as used in this invention.

Human skin equivalents (“HSE”) using biological matrices are well known in the art and may include the use of hydrated collagen gels as described by Smola et al., J Cell Biol, 122:417-29 (1993). In brief, 4 mg/mL collagen solutions are mixed at 4° C. with fibroblasts to reach a final density of 1×10⁵ cells/mL. The collagen/cell suspension is then placed on a membrane such as a filter membrane and incubated for 15 min at 37° C. in a humidified incubator to allow polymerization. Then the gel is placed in culture media of various compositions known in the art and allowed to contract and stabilize over time. All cells are either produced by methods of this invention or derived from said cells.

In addition, synthetic matrices comprising synthetic polymers may be used. Synthetic polymers include polyether urethane and polyglycan, co-polymers such as Polyactive â, Isotis N V, Bilthoven, the Netherlands), consisting of poly(ethyleneglycol-terephthatlate) (55%)/poly(butylene-terephthalate) (45%) (PEGT/PBT) copolymer and polyethylene glycol. All cells are either produced by methods of this invention or derived from said cells.

Pre-scarring (“PS”) fibroblasts may be seeded into biological or synthetic matrices at a concentration that promotes the rapid healing of wounds and/or reduces scar formation. Such concentrations range from 1.0×10⁵ to 1×10⁷ cells/cm². All cells are either produced by methods of this invention or derived from said cells.

Other tissue such as diaphragmatic tissue may also be used. All cells and tissues are either produced by methods of this invention or derived from said cells.

In another aspect of the invention, the methods of this invention result in the derivation of neural crest cells from a single cell differentiated or in the process of differentiating from pluripotent stem cells such as, but not limited to, hES, hEG, hEC, hiPS, or hED cells.

Neural crest cells derived according to the invention include neural crest cells of the forebrain or midbrain origin with no Hox gene expression as well as neural crest cells with Hox gene expression including Hoxa-1 through Hoxa-13, Hoxb-1 through Hoxb9, Hoxc-4 through Hoxc-13, and Hoxd-1 through Hoxd-13 corresponding to regions in the hindbrain, cervical, thoracic, and lumbar regions such as hindbrain cranial, vagal, cardiac, and trunk neural crest. Such varieties of neural crest cells may be pluripotent stem cells that have a propensity to differentiate into a unique constellation of cell types, though there is some plasticity here, so that given the right environmental cues, neural crest cells of one type can differentiate into the cell types normally formed by another neural crest cell type. For example, cranial neural crest cells with no Hox gene expression normally become cells and tissues including: dental mesenchyme, detal papilla, odontoblasts, dentine matrix, pulp, cementum, periodontal ligaments, chondrocytes in Meckel's cartilage, the bone of the mandible, the articulating disk of the termporomandibular joint and the branchial arch nerve ganglion, the meningens and frontal bones and suture mesenchyme of the cranium.

Generally, cranial neural crest cells have the potential to differentiate into melanocytes, nerve ganglia such as peripheral nerve ganglia such as sensory nerves and the cranial nerves, glia including Schwann cells, smooth muscle cells, cells of the ear including the bones of the middle ear, and connective tissues of the face and neck including the dermis and cells of the anterior chamber of the eye such as the endothelial cells of the cornea and cells of the lens, thymus, and parathyroid gland. The migratory nature of neural crest progenitors makes the cells particularly useful in integrating into diseased dermis such as that of EB and producing normal COL7A1 useful in the treatment of the disease.

Cardiac neural crest cells are capable of differentiating into aorticopulmonary septum, conotruncal cushions, SA node, AV node, and other conduction fibers of the heart, and derivatives of the 3rd, 4th, and 6th branchial arches.

Neural crest cells from the trunk are capable of differentiating into many of the cell types observed in cranial neural crest cells, but can also become adrenomedullary cells.

In another aspect of the invention, the methods of this invention result in the derivation of elastogenic fibroblasts with prenatal patterns of gene expression from a single cell differentiated or in the process of differentiating from pluripotent stem cells such as, but not limited to, hES, hEG, hiPS, hEC or hED cells. Such cells may be useful, for example, for the treatment of aging and sagging skin, vocal cords and the lung where age-related elastolysis may lead to disease or dysfunction.

In another aspect of the invention, the methods of this invention result in the derivation of lung connective tissue cells with prenatal patterns of gene expression that are highly elastogenic from a single cell differentiated or in the process of differentiating from pluripotent stem cells such as, but not limited to, hES, hEG, hiPS, hEC or hED cells.

In another aspect of the invention, the method comprises the derivation of 100 cells or more from a single differentiated cell or a cell in the process of differentiating from a pluripotent stem cell such as a hES cell, wherein the pluripotent stem cell is derived from the reprogramming of a somatic cell through the exposure of the somatic cell to the transcription factors to reprogram that cell to create iPS cells, or exposure of the somatic cell to cytoplasm of an undifferentiated cell (see U.S. application Nos. 60/624,827, filed Jun. 30, 1999; Ser. Nos. 09/736,268, filed Dec. 15, 2000; 10/831,599, filed Apr. 30, 2004; PCT application no. PCT/US02/18063, filed Jun. 30, 2000; U.S. application Nos. 60/314,657, filed Aug. 27, 2001; Ser. Nos. 10/228,316, filed Aug. 27, 2002; 10/487,963, filed Feb. 26, 2004; 11/055,454, filed Feb. 9, 2005; PCT application no. PCT/US02/26798, filed Aug. 27, 2002; the disclosures of which are incorporated by reference; see also U.S. application No. 60/705,625, filed Aug. 3, 2005; U.S. application No. 60/729,173, filed Oct. 20, 2005; U.S. application No. 60/818,813, filed Jul. 5, 2006; and PCT/US06/30632, filed Aug. 3, 2006, the disclosures of which are incorporated by reference).

In particular, the reprogrammed cells may be differentiated into cells with a dermatological prenatal pattern of gene expression that is highly elastogenic or capable of regeneration without causing scar formation, by methods of this invention. Dermal fibroblasts of mammalian fetal skin, especially corresponding to areas where the integument benefits from a high level of elasticity, such as in regions surrounding the joints, are responsible for synthesizing de novo the intricate architecture of elastic fibrils that function for many years without turnover. In addition, early embryonic skin is capable of regenerating without scar formation. Cells from this point in embryonic development made from the reprogrammed cells of the present invention are useful in promoting scarless regeneration of the skin including forming normal elastin architecture. This is particularly useful in treating the symptoms of the course of normal human aging, or in actinic skin damage, where there can be a profound elastolysis of the skin resulting in an aged appearance including sagging and wrinkling of the skin.

In another embodiment of the invention, the reprogrammed cells are exposed to inducers of differentiation to yield other therapeutically-useful cells such as retinal pigment epithelium, hematopoietic precursors and hemangioblastic progenitors as well as many other useful cell types of the endoderm, mesoderm, and endoderm, by methods of this invention. While some molecular pathways regulating the differentiation of embryonic progenitor cell types are understood in rudimentary form, published data demonstrates that embryonic progenitors can display a surprising plasticity in transdifferentiating into terminally differentiated cell types that would not be expected based upon their normal differentiation pathways. Therefore, the clonal purity of the cell types of the present invention, combined with their relative stability following scale up and cryopreservation, allows for the first time screens to explore the range of differentiated cell types that can be obtained from the cells of the present invention. An example of the stability of the cell lines of the present invention can be seen in the case of the cell line 4D20.8 described in Example 56. This line, after extended passage, continues to express markers of an undifferentiated embryonic mesenchymal cell and site-specific homeobox markers such as LHX8. Such differentiated cell types obtained by such screens that are more differentiated than the embryonic progenitor lines of the present invention, would have great usefulness for basic research relating to developmental biology and regenerative medicine, including drug discovery and toxicity studies, as well as in clinical transplant medicine. Such screens of differentiation potential take the basic form of thawing and culturing the cells of the present invention, exposing said cells to an array of differentiation conditions such as altered substrates, culture densities, and extracellular signals such as growth factors, cytokines, extracellular matrix components, hormones, and other factors listed in Tables I and IV herein. Such inducers include but are not limited to: cytokines such as interleukin-alpha A, interferon-alpha A/D, interferon-beta, interferon-gamma, interferon-gamma-inducible protein-10, interleukin-1-17, keratinocyte growth factor, leptin, leukemia inhibitory factor, macrophage colony-stimulating factor, and macrophage inflammatory protein-1 alpha, 1-beta, 2, 3 alpha, 3 beta, and monocyte chemotactic protein 1-3, 6kine, activin A, amphiregulin, angiogenin, B-endothelial cell growth factor, beta cellulin, brain-derived neurotrophic factor, C10, cardiotrophin-1, ciliary neurotrophic factor, cytokine-induced neutrophil chemoattractant-1, eotaxin, epidermal growth factor, epithelial neutrophil activating peptide-78, erythropoietin, estrogen receptor-alpha, estrogen receptor-beta, fibroblast growth factor (acidic and basic), heparin, FLT-3/FLK-2 ligand, glial cell line-derived neurotrophic factor, Gly-His-Lys, granulocyte colony stimulating factor, granulocyte macrophage colony stimulating factor, GRO-alpha/MGSA, GRO-beta, GRO-gamma, HCC-1, heparin-binding epidermal growth factor, hepatocyte growth factor, heregulin-alpha, insulin, insulin growth factor binding protein-1, insulin-like growth factor binding protein-1, insulin-like growth factor, insulin-like growth factor II, nerve growth factor, neurotophin-3,4, oncostatin M, placenta growth factor, pleiotrophin, rantes, stem cell factor, stromal cell-derived factor 1B, thrombopoietin, transforming growth factor-(alpha, beta1,2,3,4,5), tumor necrosis factor (alpha and beta), vascular endothelial growth factors, and bone morphogenic proteins, enzymes that alter the expression of hormones and hormone antagonists such as 17B-estradiol, adrenocorticotropic hormone, adrenomedullin, alpha-melanocyte stimulating hormone, chorionic gonadotropin, corticosteroid-binding globulin, corticosterone, dexamethasone, estriol, follicle stimulating hormone, gastrin 1, glucagons, gonadotropin, L-3,3′,5′-triiodothyronine, leutinizing hormone, L-thyroxine, melatonin, MZ-4, oxytocin, parathyroid hormone, PEC-60, pituitary growth hormone, progesterone, prolactin, secretin, sex hormone binding globulin, thyroid stimulating hormone, thyrotropin releasing factor, thyroxin-binding globulin, and vasopressin, extracellular matrix components such as fibronectin, proteolytic fragments of fibronectin, laminin, tenascin, thrombospondin, and proteoglycans such as aggrecan, heparan sulphate proteoglycan, chontroitin sulphate proteoglycan, and syndecan. Other inducers include cells or components derived from cells from defined tissues used to provide inductive signals to the differentiating cells derived from the reprogrammed cells of the present invention. Such inducer cells may derive from human, nonhuman mammal, or avian, such as specific pathogen-free (SPF) embryonic or adult cells.

After periods of time, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 days or more, the cells are analyzed for markers including but not limited to gene expression markers by microarray or PCR analysis, or immunocytochemistry for markers of differentiated cell types. Such markers are well known in the art and are displayed on web sites such as www.genepaint.org. By way of nonlimiting example, the cells of the present invention may be screened for chondrogenic potential by concentrating the cells at high density using centrifugation or micromass culture and media known to induce chondrogenesis in mesenchymal stem cells. Such screens yield surprising results with a small subset of the cells of the present invention displaying markers of cartilage formation at levels exceeding mesenchymal stem cells and normal cartilage chondrocytes (Examples 55 and 56 herein). In addition, such screens also cause the novel cell lines of the present invention to differentiate in surprising ways not previously understood. For example, the cell line 7SMOO7 responds to conditions that induce cartilage formation in mesenchymal stem cells by inducing instead the markers PAGE2, PAGE2B, PAGES, MAGEC1, MAGEC2, MAGEA1, and MAGEA10. Other differentiation condition useful in discovering additional differentiation pathways of the cells of the present invention include but are not limited to: plating cells with 10 mM β-glycerol phosphate (Sigma), 0.1 μM dexamtethasone, and 200 μM AA in αMEM medium with 10% FBS for >3 weeks; culturing cells with FGF2/EGF as a growth medium then placing the cells in medium that contains BDNF (20 ng/ml) (R&D Systems), GDNF (10 ng/ml), NGF (10 ng/ml), and 1 mM dbcAMP; expanding the cells in FGF2/EGF-containing medium than changing the medium to that which contains CNTF (10 ng/ml), neuregulin (20 ng/ml), βFGF (10 ng/ml) and 1 mM dbcAMP; the culture of the cells of the present invention with added Retinoic acid (RA) or biologically-active agonists or antagonist analogs of RA that have a wide variety of effects on different cells and appears to recapitulate embryo development and is an effective differentiation agent. Retinoic acid has been reported to differentiate “progenitors” into a wide variety of cell types including beta cells, cardiomyocytes and neural cells in a concentration dependent fashion. The most commonly used concentrations are between 10-1,000×10-9 M. For the purposes of the screen described herein, 1×10-6 M for 4-7 days may be used to ensure a differentiation effect; Phorbol esters are tumor promoters and act through protein kinase C, which, in turn, is mediated by the second messenger diacylglycerol (DAG). Phorbol esters may affect physiological cell processes more than as a differentiating agent on progenitor cells. Phorbol ester in combination with stem cell factor and endothelin-3 has been well documented to differentiate neural crest stem cells into melanocytes. The concentration range used for the present invention is 1-100×10-9 M; Cyclic AMP is a second messenger that appears to be a physiological regulator of cell processes more than as a differentiation agent. However, cAMP in conjunction with other factors, such as retinoic acid, differentiates ES cells, EG cells, and umbilical stem cells into neuronal cells. The concentrations used for the present invention are 0.1 to 1 mM; The literature on chick embryo extract is relatively old and CEE is generally used as a growth supplement for cell culture rather than as a differentiation agent. The concentrations used in the present invention are typically 1%-5% with the extracts including that made from the head, eyes, dorsal trunk, and internal organs only. An additional functional assay are conditions that promote neurosphere formation and propagation in brain-derived cells, such as:

1. Plating the cells at 50-100 cells/μl).
2. Add 0.5 ml of SFM (The medium used is SFM which is DMEM/F12 (1:1)+L glutamine & 15 mM HEPES. SFM is filtered with a 0.22 μm pore size filter after the addition of the components, with the exception of the growth factors (EGF, FGF), B-27 and ITSS which are added to the sterile SFM. Dissolve 0.096 g of Putrescine (100× stock) (1,4-Diaminobutane dihydro-chloride) in 100 ml dH2O and filter with a 0.22 μm pore size filter (store at 4° C.). Dissolve 0.00629 g of Progesterone (1000× stock) in 100 ml of dH2O and filter with a 0.22 μm pore size filter (store at 4° C.). Add 1.0M Hepes Buffer and B-27 Supplement. Add one out of hundred aliquots of Insulin-Transferrin-Sodium Selenite Supplement (ITSS) dissolved in 5.0 ml sterile dH2O (1000× stock)) containing the cells to each well of a 24 multi-well plate.
3. Incubate at 37° C. with 95% air and 5% CO.

### Passage of Neurospheres:

1. Transfer the neurospheres and medium from all wells to a 15 ml conical tube.
2. Centrifuge for 5 minutes at 200 g.
3. Remove the supernatant and add 2.0 ml of TrypLETM to the tube.
4. Use a Pasteur pipette to mix the neurospheres with the TrypLETM.
5. Place the tube in the water bath for 20 minutes at 37° C.
6. Centrifuge for 5 minutes at 500 g.
7. Remove the supernatant and re-suspend the cells in 0.5 ml of SFM.
8. Triturate with a Pasteur pipette (60-70 times)

In another embodiment of the invention, the cells with a prenatal pattern of gene expression made by methods of this invention are genetically modified to enhance a therapeutic effect, either before or after going through methods of this invention (i.e., either the parent pluripotent stem cells or the cells derived from methods of this invention). Such modifications may include the upregulation of expression of platelet-derived growth factor (PDGF) to improve wound repair when the modified cells are introduced into a wound. Such modifications may also include the up or down-regulation of one of a number of extracellular signaling molecules including, but not limited to, growth factors, cytokines, extracellular matrix components, nucleic acids encoding the foregoing, steroids, and morphogens or neutralizing antibodies to such factors. Such inducers include but are not limited to: cytokines such as interleukin-alpha A, interferon-alpha A/D, interferon-beta, interferon-gamma, interferon-gamma-inducible protein-10, interleukin-1-17, keratinocyte growth factor, leptin, leukemia inhibitory factor, macrophage colony-stimulating factor, and macrophage inflammatory protein-1 alpha, 1-beta, 2, 3 alpha, 3 beta, and monocyte chemotactic protein 1-3, 6kine, activin A, amphiregulin, angiogenin, B-endothelial cell growth factor, beta cellulin, brain-derived neurotrophic factor, C10, cardiotrophin-1, ciliary neurotrophic factor, cytokine-induced neutrophil chemoattractant-1, eotaxin, epidermal growth factor, epithelial neutrophil activating peptide-78, erythropioetin, estrogen receptor-alpha, estrogen receptor-beta, fibroblast growth factor (acidic and basic), heparin, FLT-3/FLK-2 ligand, glial cell line-derived neurotrophic factor, Gly-His-Lys, granulocyte colony stimulating factor, granulocyte macrophage colony stimulating factor, GRO-alpha/MGSA, GRO-beta, GRO-gamma, HCC-1, heparin-binding epidermal growth factor, hepatocyte growth factor, heregulin-alpha, insulin, insulin growth factor binding protein-1, insulin-like growth factor binding protein-1, insulin-like growth factor, insulin-like growth factor II, nerve growth factor, neurotophin-3,4, oncostatin M, placenta growth factor, pleiotrophin, rantes, stem cell factor, stromal cell-derived factor 1B, thrombopoietin, transforming growth factor-(alpha, beta1,2,3,4,5), tumor necrosis factor (alpha and beta), vascular endothelial growth factors, and bone morphogenic proteins, enzymes that alter the expression of hormones and hormone antagonists such as 17B-estradiol, adrenocorticotropic hormone, adrenomedullin, alpha-melanocyte stimulating hormone, chorionic gonadotropin, corticosteroid-binding globulin, corticosterone, dexamethasone, estriol, follicle stimulating hormone, gastrin 1, glucagons, gonadotropin, L-3,3′,5′-triiodothyronine, leutinizing hormone, L-thyroxine, melatonin, MZ-4, oxytocin, parathyroid hormone, PEC-60, pituitary growth hormone, progesterone, prolactin, secretin, sex hormone binding globulin, thyroid stimulating hormone, thyrotropin releasing factor, thyroxin-binding globulin, and vasopressin, extracellular matrix components such as fibronectin, proteolytic fragments of fibronectin, laminin, tenascin, thrombospondin, and proteoglycans such as aggrecan, heparan sulphate proteoglycan, chontroitin sulphate proteoglycan, and syndecan.

The present invention also provides for methods for direct differentiation of these cells from embryos without making ES cell lines (ED cells). Direct differentiation refers, for example, to methods of making downstream stem cells from an embryo without making ES cells (see U.S. patent publication no. 20050265976, published Dec. 1, 2005, and international patent publication no. WO0129206, published Apr. 26, 2001, the disclosures of which are hereby incorporated by reference). Also, direct differentiation may be accomplished from other pluripotent cells such as NT-derived, parthenote-derived, morula or blastomere-derived, cells that are homozygous in the HLA, those put into the gene trap system (see U.S. application Ser. Nos. 10/227,282, filed Aug. 26, 2002 and 10/685,693, filed October 2003, the disclosures of which are incorporated herein by reference), those made by dedifferentiating using cytoplasmic transfer (see U.S. application Ser. Nos. 10/831,599, filed Apr. 23, 2004; 10/228,316, filed Aug. 27, 2002; and 10/228,296, filed Aug. 27, 2002, the disclosures of which are incorporated herein by reference). All of these pluripotent cells may be used as the starting cells of the methods of this invention.

The present invention also provides for methods for the treatment of dermatological diseases or disorders, and one such method is the derivation of dermal cells with prenatal patterns of gene expression which may be derived according to the methods of this invention. Specifically this may be done by culturing embryo-derived cells, NT-derived, parthenote-derived, morula or blastomere-derived cells according to the methods of this invention.

The present invention also provides for a method of conducting a pharmaceutical business by establishing regional centers comprising the cells of the present invention. In one aspect of the invention, the method comprises the derivation from a subject of populations of two or more, preferably one hundred or more cells from a single cell differentiated or in the process of differentiating from pluripotent stem cells such as, but not limited to, hES, hEG, hiPS, hEC or hED cells, wherein the resulting single cell-derived population of cells can be documented not to have contaminating cells from the original parent pluripotent stem cells (such as ES, EG, EC or ED cells), wherein the resulting single cell-derived population of cells are isolated from a heterogeneous population from said subject and can be used in cell therapy in said subject.

The present invention also provides for a method of conducting a pharmaceutical business wherein the single or oligoclonal-derived populations of cells generated by the methods of the invention are marketed to healthcare providers, researchers or directly to subjects in need of such cells. One aspect provides a method for conducting a pharmaceutical business, comprising marketing to healthcare providers, researchers or to patients in need of such single or oligoclonal-derived populations of cells, the benefits of using any of the cells described herein in the treatment of a disease or disorder. A related aspect provides a method for conducting a pharmaceutical business, comprising: (a) manufacturing any of the cells described herein; and (b) marketing to healthcare providers, researchers or to patients in need of such cells the benefits of using the cells in the treatment of a disease or disorder. In some embodiments, the rights to develop and market such single or oligoclonal-derived populations of cells or to conduct such manufacturing steps may be licensed to a third party for consideration. In certain embodiments of the invention, the cells are marketed along with other factors including, but not limited to, the extracellular matrix and the gene expression profile of said cells as well as information which displays the relation of the marketed cells with other cells manufactured using the present invention and other cells used by researchers.

Other aspects of the invention include methods of doing business. Thus, this invention provides a method of doing business of identifying cell lineage by comparison of gene expression data of a cell sample of unknown cell lineage to a proprietary database of gene expression data of cell samples of known cell lineage. Example 29 describes one way of practicing this method, including a method for determining the similarity of a cell line of unknown lineage with the cell lines in the database.

In certain embodiments, the methods of the invention could be performed in a high throughput format using techniques known to one skilled in the art (see, e.g., Meldrum (2000) Genome Research Vol. 10, Issue 8, 1081-1092). The automation of the steps of the procedure using robotics could further enhance the number of conditions that can be tested. For example, 96-well microtiter plates or higher well densities such as 384- and 1536-well formats can be utilized for tissue culture techniques. Also of potential use in this invention are automated spotting, colony-picking robots or liquid handling devices. Most of these devices use an X-Y-Z robot arm (one that can move in three dimensions) mounted on an anti-vibration table. The robot arm may hold nozzles in case of non-contact spotting. In contact spotting, the robot arm may hold pins. Nozzles or pins are dipped into a first microtiter plate to pick up the test media component or cells to be delivered. The tips in case of pins are then moved to the solid support surface and allowed to touch the surface only minimally; the solution is then transferred. The pins are then washed and moved to the next set of wells and test media. This process is repeated until hundreds or thousands of test conditions are tested. One example of a robotic platform is the CellMate robotic platform.

In certain embodiments, to obtain cultures with single cells or oligoclonal clusters of multiple cells, the cells (such as the population or heterogeneous population of cells) are plated at limiting dilution. Limiting dilution may be performed as is known to one skilled in the art (Moretta et al., J Immunol. (1985) 134(4):2299-304). In certain embodiments, limiting dilution is performed such that most wells have a single cell. In other embodiments, limiting dilution is performed such that most wells have a single oligoclonal clusters of multiple cells.

Cells and compositions obtained from the methods of this invention may be tested for the capacity to be scaled up in roller bottles before being designated a product candidate.

### Applications

The disclosed methods for the culture of animal cells and tissues are useful in generating cells or progeny thereof in mammalian and human cell therapy, such as, but not limited to, generating human cells useful in treating dermatological, retinal, cardiac, neurological, endocrinological, muscular, skeletal, articular, hepatic, neurological, renal, gastrointestinal, pulmonary, and blood and vascular cell disorders in humans and nonhuman animals.

In certain embodiments of the invention, single cell-derived and oligoclonal cell-derived cells, derived by methods of this invention, are utilized in research and treatment of disorders relating to cell biology, cell-based drug discovery and in cell therapy. The single cell-derived cell populations derived using the methods of the present invention may already have received the requisite signals to be directed down a differentiation pathway. For example, some paraxial or somatopleuric single cell-derived populations of cells may express genes consistent with dermal fibroblast gene expression, in particular, a prenatal pattern of gene expression useful in promoting scarless wound repair and in promoting elastogenesis. Such cells include, for example, those cells listed in Table II, including but not limited to: cells of the heart; cells of the musculo-skeletal system; cells of the nervous tissue; cells of the respiratory system; cells of the endocrine system; cells of the vascular system; cells of the hematopoietic system; cells of the integumentary system; cells of the urinary system; or cells of the gastrointestinal system. Such cells may be stably grafted in a histocompatible host when the cells are grafted into the tissue into which the cells would normally differentiate. Such final differentiated tissues are well known from the art of embryology and by way of nonlimiting example, some are listed in Table III. Such tissues include for example (as listed in Table III), but not limited to: endoderm-embryonic tissues; mesoderm-embryonic tissues; ectoderm-embryonic tissues; or extraembryonic cells.

In certain embodiments of the invention, single cell-derived and oligoclonal cell-derived cells are introduced into the tissues in which they normally reside in order to exhibit therapeutic utility. For example, the clonogenic populations of cells derived by methods of this invention may be introduced into the tissues including but not limited to the tissues listed in Table II.

In certain embodiments of the invention, single cell-derived and oligoclonal cell-derived cells, derived by methods of this invention, are utilized in inducing the differentiation of other pluripotent stem cells. The generation of single cell-derived populations of cells capable of being propagated in vitro while maintaining an embryonic pattern of gene expression is useful in inducing the differentiation of other pluripotent stem cells. Cell-cell induction is a common means of directing differentiation in the early embryo. Many potentially medically-useful cell types are influenced by inductive signals during normal embryonic development, including spinal cord neurons, cardiac cells, pancreatic beta cells, and definitive hematopoietic cells. Single cell-derived populations of cells capable of being propagated in vitro while maintaining an embryonic pattern of gene expression can be cultured in a variety of in vitro, in ovo, or in vivo culture conditions to induce the differentiation of other pluripotent stem cells to become desired cell or tissue types.

Induction may be carried out in a variety of methods that juxtapose the inducer cell with the target cell. By way of nonlimiting examples, the inducer cells may be plated in tissue culture and treated with mitomycin C or radiation to prevent the cells from replicating further. The target cells are then plated on top of the mitotically-inactivated inducer cells. Alternatively, single cell-derived inducer cells may be cultured on a removable membrane from a larger culture of cells or from an original single cell-derived colony and the target cells may be plated on top of the inducer cells or a separate membrane covered with target cells may be juxtaposed so as to sandwich the two cell layers in direct contact. The resulting bilayer of cells may be cultured in vitro, transplanted into a SPF avian egg, or cultured in conditions to allow growth in three dimensions while being provided vascular support (see, for example, international patent publication number WO2005068610, published Jul. 28, 2005, the disclosure of which is hereby incorporated by reference). The inducer cells may also be from a source of pluripotent stem cells, including hES or hED cells, in which a suicide construct has been introduced such that the inducer cells can be removed at will. Cell types useful in single cell-derived and oligoclonal cell-derived induction may include cases of induction well known in the art to occur naturally in normal embryonic development.

In certain embodiments of the invention, single cell-derived cells and oligoclonal cell-derived cells, derived by methods of this invention, are used as “feeder cells” to support the growth of other cell types, including pluripotent stem cells. The use of single cell-derived cells and oligoclonal cell-derived cells of the present invention as feeder cells alleviates the potential risk of transmitting pathogens from feeder cells derived from other mammalian sources to the target cells. The feeder cells may be inactivated, for example, by gamma ray irradiation or by treatment with mitomycin C, to limit replication and then co-cultured with the pluripotent stem cells.

In certain embodiments of the invention, the extracellular matrix (ECM) of single cell-derived and oligoclonal cell-derived cells, derived by methods of this invention, may be used to support less differentiated cells (see Stojkovic et al., Stem Cells (2005) 23(3):306-14). Certain cell types that normally require a feeder layer can be supported in feeder-free culture on a matrix (Rosier et al., Dev Dyn. (2004) 229(2):259-74). The matrix can be deposited by preculturing and lysing a matrix-forming cell line (see WO 99/20741), such as the STO mouse fibroblast line (ATCC Accession No. CRL-1503), or human placental fibroblasts.

In certain embodiments of the invention, the conditioned media of single cell-derived and oligoclonal cell-derived cell cultures may be collected, pooled, filtered and stored as conditioned medium. This conditioned medium may be formulated and used for research and therapy. Such conditioned medium may contribute to maintaining a less differentiated state and allow propagation of cells such as pluripotent stem cells. In certain embodiments of the invention, conditioned medium of single cell-derived and oligoclonal cell-derived cell cultures derived by the methods of this invention can be used to induce differentiation of other cell types, including pluripotent stem cells. The use of conditioned medium of single cell-derived and oligoclonal cell-derived cell cultures may be advantageous in reducing the potential risk of exposing cultured cells to non-human animal pathogens derived from other mammalian sources (i.e. xenogeneic free).

In another embodiment of the invention, single cell-derived and oligoclonal cell-derived paraxial mesoderm, neural crest mesenchyme, or somatopleuric mesoderm, derived by methods of this invention, can be used to induce embryonic ectoderm or single cell-derived embryonic ectoderm into keratinocytes for use in skin research and grafting for burns, wound repair, and drug discovery.

In another embodiment of the invention, the use of single cell-derived and oligoclonal cell-derived prechordal plate mesoderm, derived by methods of this invention, to induce embryonic ectoderm or single cell-derived or oligoclonal cell-derived embryonic ectoderm into neuroectodermal cells capable of generating CNS cells, may be useful in neuron research and grafting for neurodegenerative diseases, as well as drug discovery. The single cell-derived and oligoclonal cell-derived prechordal plate mesoderm can be identified by transcript analysis as described herein through the expression of, for example, lim-1.

In another embodiment of the invention, the single cell-derived and oligoclonal cell-derived notochord mesodermal cells, derived by methods of this invention, are identified by their expression of brachyury. In normal development, notochordal cells induce the floor of the neural plate mesoderm (which induces the spinal chord) to make sonic hedgehog (“SHH”), a ventralizing signal, that induces the floor of the neural tube to express SHH as well, which induces the expression of FP1, FP2, and SC1 by the floor plate of the neural tube. Therefore, notochordal mesodermal cells can be used to induce neural plate ectodermal cells or neural tube neuroepithelial cells to differentiate into spinal cord neurons. Such neurons may be identified and confirmed by assaying the gene expression assays described herein for cells expressing FP1, FP2, or SC1. These cells expressing one or more of these markers could be useful in spinal cord regeneration.

Our discovery that various single cell-derived and oligoclonal cell-derived cells in early embryonic lineages may be propagated without the loss of their embryonic phenotype allows numerous types of mesodermal inducer cells to induce differentiation in embryonic ectoderm or endoderm. However, single cell-derived and oligoclonal cell-derived cells from endoderm and ectodermal lineages, derived by methods of this invention, may be useful in induction as well. For example, surface ectoderm and notochord express Shh and thereby induce somites to become sclerotome mesodermal cells that express M-twist and Pax-1 and surface ectoderm. Also, as another example, notochord expresses extracellular proteins of the Wnt family and thereby induces other somite mesodermal cells to become dermatome mesodermal cells that express gMHox, and dermo-1. Meanwhile, the myotome expresses N-myc and myogenin.

The juxtaposition of the inducer and target cells provides a useful in vitro model of differentiation that can be used for research into early embryonic differentiation, for drug screening, and for studies of teratology. The target cells differentiated by the single cell-derived inducer cells may also be used for research, drug discovery, and cell-based therapy.

In certain embodiments of the invention, the single cell-derived and oligoclonal cell-derived cells, derived by methods of this invention, may be used to generate skin equivalents, as well as to reconstitute full-thickness human skin, according to the methods described in U.S. application Ser. No. 09/037,191, filed Mar. 9, 1998 (U.S. publication no. 20010048917, published Dec. 6, 2001); 10/013,124, filed Dec. 7, 2001 (U.S. publication no. 20020120950, published Aug. 29, 2002); 10/982,186, filed Nov. 5, 2004 (U.S. publication no. 20050118146, published Jun. 2, 2005); the disclosure of each of which is incorporated herein by reference. For example, the single cell-derived and oligoclonal cell-derived cells may be incorporated into a layered cell sorted tissue that includes a discrete first cell layer and a discrete second cell layer that are formed in vitro by the spontaneous sorting of cells from a homogenous cell mixture. The first cell layer may include any cell type, but preferably includes epithelial cells, in particular, keratinocytes. Other cell types that may be used in the first cell layer are CaCo2 cells, A431 cells, and HUC18 cells. The second cell layer may also include cells of any type, but preferably includes mesenchymal cells, in particular, fibroblasts. The layered cell sorted tissue possesses an epidermal-dermal junction that is substantially similar in structure and function to its native counterpart. That is, the tissue expresses the necessary integral proteins such as hemidesmosomes and collagen I, collagen IV, and collagen VII, to attach the epidermal and dermal layers with the proper basement membrane morphology. The single cell-derived and oligoclonal cell-derived cells may then sort to form an epidermal layer that contacts the connective tissue component. The layered cell sorted tissues comprising the single cell-derived and oligoclonal cell-derived cells may be used as a skin graft that could be used on graft sites such as traumatic wounds and burn injury.

In another embodiment of the invention, single cell-derived and oligoclonal cell-derived cells of this invention may be used as a means to identify and characterize genes that are transcriptionally activated or repressed as the cells undergo differentiation. For example, libraries of gene trap single cell-derived or oligoclonal cell-derived cells may be made by methods of this invention, and assayed to detect changes in the level of expression of the gene trap markers as the cells differentiate in vitro and in vivo. The methods for making gene trap cells and for detecting changes in the expression of the gene trap markers as the cells differentiate are reviewed in Durick et al. (Genome Res. (1999) 9:1019-25), the disclosure of which is incorporated herein by reference). The vectors and methods useful for making gene trap cells and for detecting changes in the expression of the gene trap markers as the cells differentiate are also described in U.S. Pat. No. 5,922,601 (Baetscher et al.), U.S. Pat. No. 6,248,934 (Tessier-Lavigne) and in U.S. patent publication No. 20040219563 (West et al.), the disclosures of which are also incorporated herein by reference. Methods for genetically modifying cells, inducing their differentiation in vitro, and using them to generate chimeric or nuclear-transfer cloned embryos and cloned mice are developed and known in the art. To facilitate the identification of genes and the characterization of their physiological activities, large libraries of gene trap cells having gene trap DNA markers randomly inserted in their genomes may be prepared. Efficient methods have been developed to screen and detect changes in the level of expression of the gene trap markers as the cells differentiate in vitro or in vivo. In vivo methods for inducing single cell-derived or oligoclonal cell-derived cells to differentiate further include injecting one or more cells into a blastocyst to form a chimeric embryo that is allowed to develop; fusing a stem cell with an enucleated oocyte to form a nuclear transfer unit (NTU), and culturing the NTU under conditions that result in generation of an embryo that is allowed to develop; and implanting one or more clonogenic differentiated cells into an immune-compromised or a histocompatible host animal (e.g., a SCID mouse, or a syngeneic nuclear donor) and allowing teratomas comprising differentiated cells to form. In vitro methods for inducing single cell-derived or oligoclonal cell-derived cells to differentiate further include culturing the cells in a monolayer, in suspension, or in three-dimensional matrices, alone or in co-culture with cells of a different type, and exposing them to one of many combinations of chemical, biological, and physical agents, including co-culture with one or more different types of cells, that are known to capable of induce or allow differentiation.

In another embodiment of the invention, cell types that do not proliferate well under any known cell culture conditions may be induced to proliferate such that they can be isolated clonally or oligoclonally according to the methods of this invention through the regulated expression of factors that overcome inhibition of the cell cycle, such as regulated expression of SV40 virus large T-antigen (Tag), or regulated E1a and/or E1b, or papillomavirus E6 and/or E7. To artificially stimulate the proliferation of such cell lines produced using the methods of the present invention, pluripotent stem cells such as hES cells may be transfected with a plasmid construct containing a temperature sensitive mutant of SV40 Tag regulated by a gamma-interferon promoter (Jat et al., Proc Natl Acad Sci USA 88:5096-5100 (1991)). The inducible Tag hES cells are then allowed to undergo a first round of differentiation with Tag in the uninduced state at the nonpermissive temperature of 37° C. and in medium lacking exogenous gamma-interferon in six differing conditions. For some cells that have potential for therapeutic or other commercial applications it may be desirable to remove the ectopic SV40 Tag DNA sequences. This may be accomplished by flanking the Tag and other undesirable DNA sequences with the recognition sequences for the Cre or FLP site specific recombinases (Sargent and Wilson, Recombination and Gene Targeting in Mammalian Cells. Current Research in Molecular Therapeutics (1998) 1:584-590). When these recombinases are expressed in cells they efficiently catalyze recombination at a high frequency, specifically between DNA containing their respective recognition sequences. For example, genes flanked by the loxp recognition sequence for the Cre recombinase may be specifically deleted on intracellular transient expression of Cre recombinase.

For example, construction of H-2Kb-tsA58/neo and H-2Kb-tsA58/neo/loxp vectors may involve the 5′ flanking promoter sequences and the transcriptional initiation site of the mouse H-2Kb classl gene being fused to the SV40 tsA58 early region coding sequences. The 4.2-kilobase (kb) EcoRI-Nru I fragment encompassing the H-2Kb promoter sequences are ligated to the 2.7-kb Bgl I-BamHI fragment derived from the tsA58 early region gene and pUC19 double-digested with EcoRI and BamHI. The Bgl I site is blunted by using the Klenow fragment of Escherichia coli DNA polymerase Ito allow fusion to the Nru I site to generate the Tag expression vector pH-2Kb-tsA58 (Jat et al., Proc Natl Acad Sci USA 88:5096-5100 (1991)). To create a drug selectable Tag vector, the MC1NeoPolA expression cassette is isolated from the pMC1NeoPolA vector as a XhoI/SalI fragment and subcloned into Sail linearized H-2Kb-tsA58 vector to generate pH-2Kb-tsA58/neo. To create a pH-2Kb-tsA58/neo vector which has the pH-2Kb-tsA58/neo cassettes flanked by loxp site-specific recombination sequences, two loxp oligonucleotide duplexes are synthesized and ligated into pH-2Kb-tsA58/neo vector in the unique EcoRI and SalI sites that flank the expression cassettes and in an orientation that allow deletion of the expression cassettes on recombination. Each oligonucleotide duplex reconstructs a functional restriction site and an inactive restriction site such that the entire loxpH-2Kb-tsA58/neoloxp cassette can be removed intact by restriction endonuclease digestion with EcoRI and SalI. To construct this vector, a DNA oligonucleotide duplex molecule containing the loxp recognition sequence (Hoess et al., Proc Natl Acad Sci USA (1982) 79(11): 3398-402) and single stranded ends complementary to restriction endonuclease EcoRI-cut DNA is ligated into EcoRI digested pH-2Kb-tsA58/neo vector to create the ploxpH-2Kb-tsA58/neo vector. A similar loxp oligonucleotide duplex containing single stranded ends complementary to restriction endonuclease SalI-cut DNA is ligated into Sail digested ploxpH-2Kb-tsA58/neo vector to create the ploxpH-2 Kb-tsA58/neoloxp vector. Prior to transfection into H9 hES cells the pH-2Kb-tsA58/neo vector or ploxpH-2Kb-tsA58/neoloxp vector is linearized by restriction endonuclease digestion with EcoRI.

Transfection and establishment of transgenic cell lines may be performed by creating H9 hES cell lines or other ES cells with stably integrated temperature sensitive Tag by transfecting linearized plasmid vector by electroporation or using the chemical transfection reagent Exgene 500 transfection system (Frementas) as previously described (Eiges et al., Current Biol, 11:514-518 (2001), Zwaka and Thomson, Nat. Biotechnol. 21:319-321 (2003) and stable transfectants selected in the presence of the neomycin analog G418.

Transfection and establishment of transgenic cell lines may also be performed by chemical transfection. Human H9 ES cells or other ES cells are transfected with linearized pH-2Kb-tsA58/neo using the ExGen 500 transfection system (Fermentas). Transfection of human ES cells is carried out in 6-well tissue culture plates two days after plating on MEFs, using established conditions described above, and is performed as described by the manufacturer's protocol. Specifically, 2 ug of plasmid DNA plus 10 ul of the transfecting agent ExGen 500 is added to about 3×10⁵ cells/well in a final volume of 1 ml medium per well. The 6-well tissue culture plates are centrifuged at 280×g for 5 minutes and incubated at 37° C. in a humidified low oxygen incubator for an additional 45 min. Residual transfection agent is removed by washing the cells twice with PBS. The following day, cells are trypsinized and approximately 5×10⁵ cells are replated per 10 cm culture dish containing inactivated neomycin resistant MEF cells. Two days following replating, the neomycin analog G418 (200 ng/ml) is added to the growth medium. After approximately 10-14 days, G418 resistant colonies are observed. Single transgenic colonies are picked by a micropipette, dissociated into small clumps of cells, and transferred into a 24-well culture dish containing neomycin resistant MEF cells. The G418 resistant H9 cells are expanded before storage in liquid nitrogen or used for differentiation.

Transfection and establishment of transgenic cell lines may also be performed by electroporation. H9 hES cells or other ES cells are harvested by gentle trypsinization (0.05% mg/ml; Invitrogen, Carlsbad, Calif.), taking care to minimize dissociation into single cell suspensions. Cells are washed with MEF medium, and resuspended in 0.5 ml hES culture medium, not containing antibiotics, at a concentration of 1.5-3.0×10⁷ cells/ml. Immediately prior to electroporation, 40 μg of linearized vector DNA is added in a volume less than 80 ul, and 0.8 ml of the DNA/cell suspension is added to each electroporation cuvette (0.4 cm gap cuvette; BioRad, Hercules, Calif.). Cells are electroporated with a single 320 V, 200 uF pulse at room temperature using the BioRad Gene Pulser II. Electroporated cells are incubated for 10 minutes at room temperature and the contents of each cuvette plated at high density on a 10 cm culture dish seeded with neomycin resistant MEF cells. G418 selection (50 μg/ml, Invitrogen) is started 48 hours after electroporation. After approximately two weeks of G418 selection, surviving colonies are picked using a micropipette to dissociate nascent colonies into small cell clumps and transferred into 24-well tissue culture plates seeded with neomycin resistant MEF cells in hES medium containing 50 ug/ml G418. The G418 resistant colonies are expanded before individual analysis by PCR using primers specific for the neomycin resistance cassette and for the SV40 large T antigen, storage in liquid nitrogen, or used for differentiation. PCR positive clones are rescreened by Southern blot analysis for confirmation using genomic DNA isolated from G418 resistant clones and hybridizing with radiolabelled probes from the neomycin cassette or the SV40 large T antigen.

Inducible Tag-expressing cells are plated in a standard 6 well tissue culture plate on a feeder layer of mouse embryonic fibroblasts and allowed to grow for 9 days to confluence. The hES cell growth medium is replaced by any of the combinations of specialized media or other culture conditions described herein (see Table I) and the hES cells are allowed to differentiate under a variety of conditions and for variable periods of time as described herein.

The resulting heterogeneous mixture of cells is then rinsed with phosphate buffered saline, dissociated into single cells such as with trypsin (0.25% trypsin) and the differentiated cells plated out so as to allow clonal or oligoclonal growth as described herein. The differentiated cells are allowed to proliferate for 14-20 days under permissive temperature and the resulting colonies are cloned and plated in 24 well plates containing the same medium supplemented with gamma-interferon under the permissive temperature of 32.5° C. and extracellular matrix from which they were derived. The cloned colonies are expanded to obtain a stock of cells and the cell line stocks are cryopreserved. To determine the pattern of gene expression, the cells are shifted to the same medium reduced in serum concentration by 20-fold, free of gamma interferon, and at the nonpermissive temperature of 37° C. for five days.

Removal of H-2Kb-tsA58/neo Vector Sequences from Cell Lines

To remove the H-2Kb-tsA58/neo expression cassettes from cells, cells are transfected with an expression cassette for the Cre, FLP, or equivalent recombinase, for example the pCX-NLS-Cre expression vector containing a nuclear localization signal fused in frame with Cre recombinase. Cells are transfected with Cre expression vector by electroporation or chemical transfection reagents, for example the ExGen 500 transfection system (Fermentas). Transfection of human ES-derived cells is carried out in 6-well tissue culture plates, using established conditions described above, and is performed as described by the manufacturer's protocol. Specifically, 2 μg of Cre expression vector DNA plus 10 μl of the transfecting agent ExGen 500 is added to about 3×10⁵ cells/well in a final volume of 1 ml medium per well. The 6-well tissue culture plates are centrifuged at 280×g for 5 minutes and incubated at 37° C. in a humidified low oxygen incubator for an additional 45 min. Residual transfection agent is removed by washing the cells twice with PBS. The following day, cells are trypsinized and replated at a density of approximately 1000 cells/10 cm culture dish or at a density of approximately 1 cell/well of a 96-well tissue culture plate. Each colony growing on 10 cm tissue culture plates are picked into individual wells of a 96-well plate several weeks after replating. Cells are screened by PCR for loss of H-2Kb-tsA58/neo sequences and by sensitivity to the drug G418. Loss of H-2Kb-tsA58/neo sequences are confirmed by southern analysis using ³²P labeled probes from the H-2Kb-tsA58/neo cassette (Sambrook and Russell, Molecular Cloning: A Laboratory Manual, 3^{rd} Edition, 2001, Cold Spring Harbor Press).

In another embodiment of the invention, the factors that override cell cycle arrest may be fused with additional proteins or protein domains and delivered to the cells. For example, factors that override cell cycle arrest may be joined to a protein transduction domain (PTD). Protein transduction domains, covalently or non-covalently linked to factors that override cell cycle arrest, allow the translocation of said factors across the cell membranes so the protein may ultimately reach the nuclear compartments of the cells. PTDs that may be fused with factors that override cell cycle arrest include the PTD of the HIV transactivating protein (TAT) (Tat 47-57) (Schwarze and Dowdy 2000 Trends Pharmacol. Sci. 21: 45-48; Krosl et al. 2003 Nature Medicine (9): 1428-1432). For the HIV TAT protein, the amino acid sequence conferring membrane translocation activity corresponds to residues 47-57 (Ho et al., 2001, Cancer Research 61: 473-477; Vives et al., 1997, J. Biol. Chem. 272: 16010-16017). These residues alone can confer protein translocation activity.

In another embodiment of the invention, the PTD and the cycle arrest factor may be conjugated via a linker. The exact length and sequence of the linker and its orientation relative to the linked sequences may vary. The linker may comprise, for example, 2, 10, 20, 30, or more amino acids and may be selected based on desired properties such as solubility, length, steric separation, etc. In particular embodiments, the linker may comprise a functional sequence useful for the purification, detection, or modification, for example, of the fusion protein.

In another embodiment of the invention, single cell-derived or oligoclonal cell-derived cells of this invention may be reprogrammed to an undifferentiated state through novel reprogramming technique, as described in U.S. application No. 60/705,625, filed Aug. 3, 2005, U.S. application No. 60/729,173, filed Oct. 20, 2005; U.S. application No. 60/818,813, filed Jul. 5, 2006, the disclosures of which are incorporated herein by reference. Briefly, the cells may reprogrammed to an undifferentiated state using at least a two, preferably three-step process involving a first nuclear remodeling step, a second cellular reconstitution step, and finally, a third step in which the resulting colonies of cells arising from step two are characterized for the extent of reprogramming and for the normality of the karyotype and quality. In certain embodiments, the single cell-derived or oligoclonal cell-derived cells of this invention may be reprogrammed in the first nuclear remodeling step of the reprogramming process by remodeling the nuclear envelope and the chromatin of a differentiated cell to more closely resemble the molecular composition of an undifferentiated or a germ-line cell. In the second cellular reconstitution step of the reprogramming process, the nucleus, containing the remodeled nuclear envelope of step one, is then fused with a cytoplasmic bleb containing requisite mitotic apparatus which is capable, together with the transferred nucleus, of producing a population of undifferentiated stem cells such as ES or ED-like cells capable of proliferation. In the third step of the reprogramming process, colonies of cells arising from one or a number of cells resulting from step two are characterized for the extent of reprogramming and for the normality of the karyotype and colonies of a high quality are selected. While this third step is not required to successfully reprogram cells and is not necessary in some applications, the inclusion of the third quality control step is preferred when reprogrammed cells are used in certain applications such as human transplantation. Finally, colonies of reprogrammed cells that have a normal karyotype but not sufficient degree of programming may be recycled by repeating steps one and two or steps one through three.

In another embodiment of the invention, the single cell-derived and oligoclonal cell-derived cells may be used to generate ligands using phage display technology (see U.S. application No. 60/685,758, filed May 27, 2005, and PCT US2006/020552, filed May 26, 2006, the disclosures of which are hereby incorporated by reference).

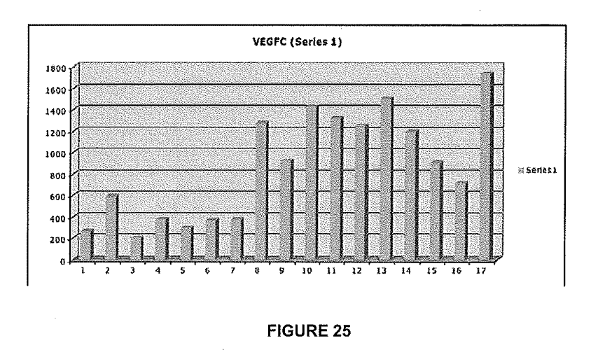

In another embodiment of the invention, the single cell-derived or oligoclonal cell-derived cells of this invention may exhibit unique patterns of gene expression such as high levels of angiogenic and neurotrophic factors. Such cells may be useful for the delivery of these factors to tissues to promote vascularization or innervation where those responses are therapeutic. For example, in the case of the angiogenic factors, cell lines that express high levels of such factors including VEGFA, B, C, or D or angiopoietin-1 or -2 can be transplanted using delivery technologies appropriate to the target tissue to deliver cells that express said angiogenic factor(s) to induce angiogenesis for therapeutic effect. As an example, FIG. 25 depicts the relative gene expression of the angiogenic factor VEGFC in the cells derived from clones 1-17 of Series 1.

The expression of genes of the cells of this invention may be determined. Measurement of the gene expression levels may be performed by any known methods in the art, including but not limited to, microarray gene expression analysis, bead array gene expression analysis and Northern analysis. The gene expression levels may be represented as relative expression normalized to the ADPRT or GAPD housekeeping genes. Based on the gene expression levels, one would expect the expression of the corresponding proteins by the cells of the invention. For example, in the case of cell clone ACTC60 (or B-28) of Series 1, relatively high levels of DKK1, VEGFC and IL1R1 were observed. Therefore, the ability to measure the bioactive or growth factors produced by said cells may be useful in research and in the treatment of disease.

The formulation and dosage of said cells will vary with the tissue and the disease state but in the case of humans and most veterinary animals species, the dosage will be between 10²-10⁶ cells and the formulation can be, by way of nonlimiting example, a cell suspension in isosmotic buffer or a monolayer of cells attached to an layer of extracellular matrix such as contracted gelatin.

In the case of neutrophic factors, the cells made by the methods of this invention may be used to induce the innervation of tissue such as to improve the sensory innervation of the skin in wound repair or regeneration, or other sensory or motor innervation. For example, the cell clone number 1 (ACTC61/B30) described in Example 32 displays a high level of expression of pleiotrophin (PTN) and may therefore be formulated for this use using delivery and formulation technologies well known in the art including by way of nonlimiting example, humans and veterinary animal applications where the dosage will be between 10²-10⁶ cells and the formulation can be, by way of nonlimiting example, a cell suspension in isosmotic buffer or a monolayer of cells attached to an layer of extracellular matrix such as contracted gelatin.

Such use of cells that promote angiogenesis or neurite outgrowth may further be combined with an adjunct therapy that includes young hemangioblasts or angioblasts in the case of angiogenesis or neuronal precursors of various kinds in the case of neurite outgrowth. Such combined therapy may have particular utility where the mere administration of angiogenic factors or neurite outgrowth promoting factors by themselves are not sufficient to generate a response due to the fact that there is a paucity of cells capable of responding to the stimulus.

In the case of angiogenesis, the senescence of the vascular endothelium or circulating endothelial precursor cells such as hemangioblasts may blunt the response to angiogenic stimulus. The co-administration of young hemangioblasts by various modalities known in the art based on the size of the animal and the target tissue along with cells capable of delivering an angiogenic stimulus will provide an improved angiogenic response. Such an induction of angiogenesis can be useful in promoting wound healing, the vascularization of tissues prone to ischemia such as aged myocardium, skeletal, or smooth muscle, skin (as in the case of nonhealing skin ulcers such as decubitus or stasis ulcers), intestine, kidney, liver, bone, or brain. Measurement of the gene expression levels may be performed by any known methods in the art, including but not limited to, microarray gene expression analysis, bead array gene expression analysis and Northern analysis. The gene expression levels may be represented as relative expression normalized to the ADPRT (Accession number NM_{—}001618.2), GAPD (Accession number NM_{—}002046.2), or other housekeeping genes known in the art. The gene expression data may also be normalized by a median of medians method. In this method, each array gives a different total intensity. Using the median value is a robust way of comparing cell lines (arrays) in an experiment. As an example, the median was found for each cell line and then the median of those medians became the value for normalization. The signal from the each cell line was made relative to each of the other cell lines.

In another embodiment of the invention, the single cell-derived or oligoclonal cell-derived cells of this invention may express unique patterns of CD antigen gene expression, which are cell surface antigens. The differential expression of CD antigens on the cell surface may be useful as a tool, for example, for sorting cells using commerically available antibodies, based upon which CD antigens are expressed by the cells. The expression profiles of CD antigens of some cells of this invention are shown in Table X and XI. H9-B1 and H9-B2 cell lines shown in Table X or Table XI are ES cells. The rest of the cells shown in Tables X or XI are clonal cell lines derived according to the methods of this invention. For example, there are CD antigens that are expressed in ES cells and not (or in some cases, at reduced levels) in the relatively more differentiated cell lines of this invention. This could be a very useful tool for selecting, sorting, purifying and/or characterizing ES cells. Since the CD antigens are expressed on the cell surface and antibodies to them are, generally speaking, commercially available, antibodies (or specific combinations of them) can be used to purify pure populations of ES cells or cells of this invention out of a heterogeneous mixture of cells. This could be useful in various strategies to grow ES cells or cells of this invention, or prepare these cells for various commercial purposes.

As shown in Table X, the CD antigens that show expression in ES cells (H9-B1 and H9-B2 are ES cells in Table X) and reduced or no expression in the relatively more differentiated cells of this invention include: CD41, CD100, CD107b, CD133, CD184, CD225, CD317, CD321, CD324, CD326, CD333, CD334 (see Table X). Conversely, there are several CD antigens that are robustly expressed in the relative more differentiated cells of this invention, but are not expressed in ES cells (or in some cases at markedly reduced levels). The antigens that fall into this category include: CD73, CD97, CD140B, CD151, CD172A, CD230, CD280, CDw210b (see Table X). These antigens may be useful in a negative selection strategy to grow ES cells.

Table XI shows unique “signature” of gene expression for some cell lines of this invention (Table X shows a signature for human ES cells). For example, looking at cell line 4, it is CD24 positive, CD133 positive, CD142 positive and CD339 positive (see Table XI for the signature for cell line 4). This combination of antibodies could then be used to purify or enrich for populations of cell line 4. Also, cell line 4 is the only cell line expressing CD133 (besides the ES cells in the last two columns; i.e., H9-B1 and H9-B2). The fact that the cell lines look different from each other (with respect to their CD antigen expression profile) means that there should be a unique (or semi-unique) combination of CD antibodies that can be used to enrich and/or purify these cell types from a heterogeneous mixture.

In Tables X and XI, the first three columns indicate the CD designation, its corresponding gene name and corresponding accession number, respectively. The other columns show expression levels of either cell lines of this invention (CM10-1, B-1, 4, CM50-4, B-16, 2-2, 2-1, B-28, B-7, 6-1, B-25, B-26, B-3, B-11, B-2, B-29, B-6, B-17, B-30, CM30-2, CM0-2, 2-3, CM10-4, CM20-4, CM30-5, CM50-5, CM0-5, CM0-3, B-14) or ES cells (H9-B1 and H9-B2). All the cells in Tables X and XI are human cells.

In another embodiment of the invention, the single cell-derived and oligoclonal cell-derived cells, derived by methods of this invention, may be injected into mice to raise antibodies to differentiation antigens. Antibodies to differentiation antigens would be useful for both identifying the cells to document the purity of populations for cell therapies, for research in cell differentiation, as well as for documenting the presence and fate of the cells following transplantation. In general, the techniques for raising antibodies are well known in the art.

A cell produced by the methods of this invention could produce large amounts of BMP3b, and this cell could therefore be useful in inducing bone.

In another embodiment of the invention, cells may produce large quantities of PTN (Accession number NM_{—}002825.5), MDK (Accession number NM_{—}002391.2), or ANGPT2 (Accession number NM_{—}001147.1), or other angiogenesis factors and therefore may be useful in inducing angiogenesis when injected in vivo as cell therapy, when mitotically inactivated and then injected in vivo, or when combined with a matrix in either a mitotically-inactivated or native state for use in inducing angiogenesis. PTN-producing cells described in the present invention are also useful when implanted in vivo in either a native or mitotically-inactivated state for delivering neuro-active factors, such as in preventing the apoptosis of neurons following injury to said neurons.

In another embodiment of the invention, the single cell-derived and oligoclonal cell-derived cells may be used for the purpose of generating increased quantities of diverse cell types with less pluripotentiality than the original stem cell type, but not yet fully differentiated cells. mRNA or miRNA can then be prepared from these cell lines and microarrays of their relative gene expression can be performed as described herein.

In another embodiment of the invention, the single cell-derived and oligoclonal cell-derived cells may be used in animal transplant models, e.g. transplanting escalating doses of the cells with or without other molecules, such as ECM components, to determine whether the cells proliferate after transplantation, where they migrate to, and their long-term differentiated fate in safety studies.

In another embodiment of the invention, the single cell-derived and oligoclonal cell-derived cells generated according to the methods of the present invention are useful for harvesting mRNA, microRNA, and cDNA from either single cells or a small number of cells (i.e., clones) to generate a database of gene expression information. This database allows researchers to identify the identity of cell types by searching for which cell types in the database express or do not express genes at comparable levels of the cell type or cell types under investigation. For example, the relative expression of mRNA may be determined using microarray analysis as is well known in the art. The relative values may be imported into a software such as Microsoft Excel and gene expression values from the different cell lines normalized using various techniques well known in the art such as mean, mode, median, and quantile normalization. Hierarchical clustering with the single linkage method may be performed with the software such as The R Project for Statistical Computing as is well known in the art. An example of such documentation may be found at http(colon)//sekhon(dot)berkeley(dot)edu/stats/html/hclust.html.

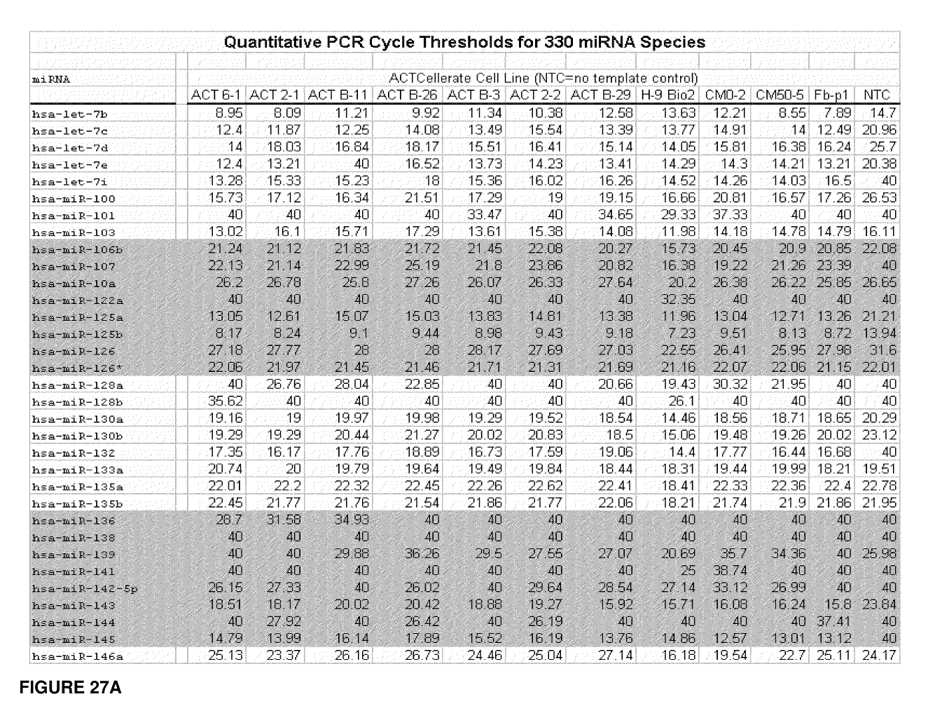

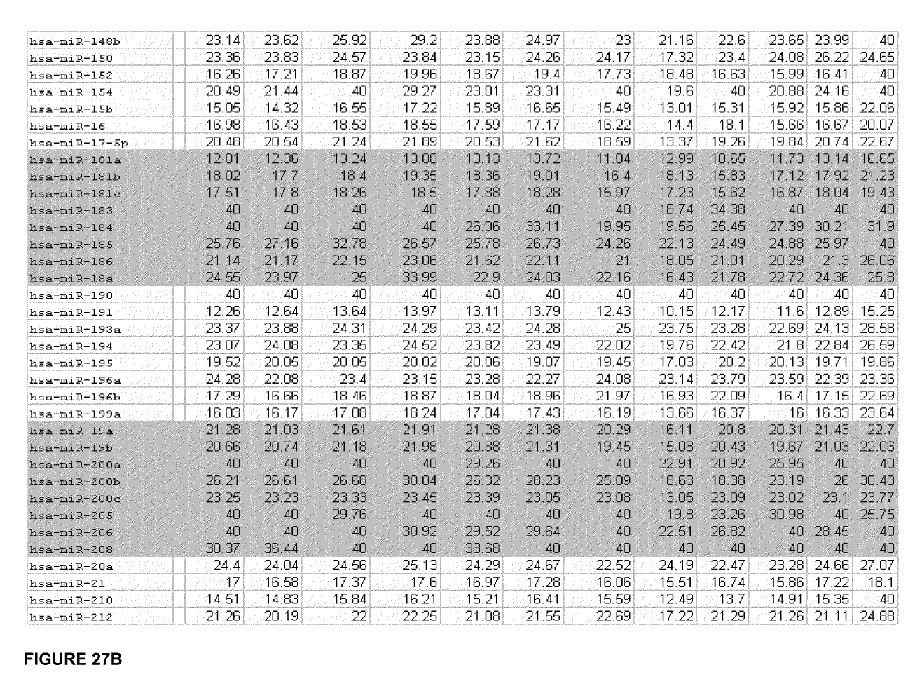

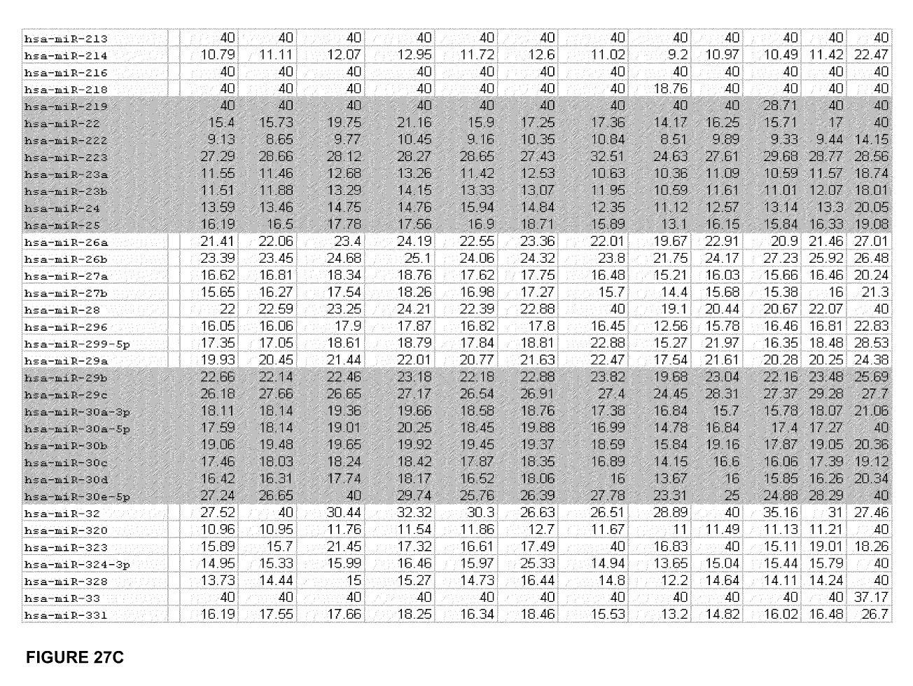

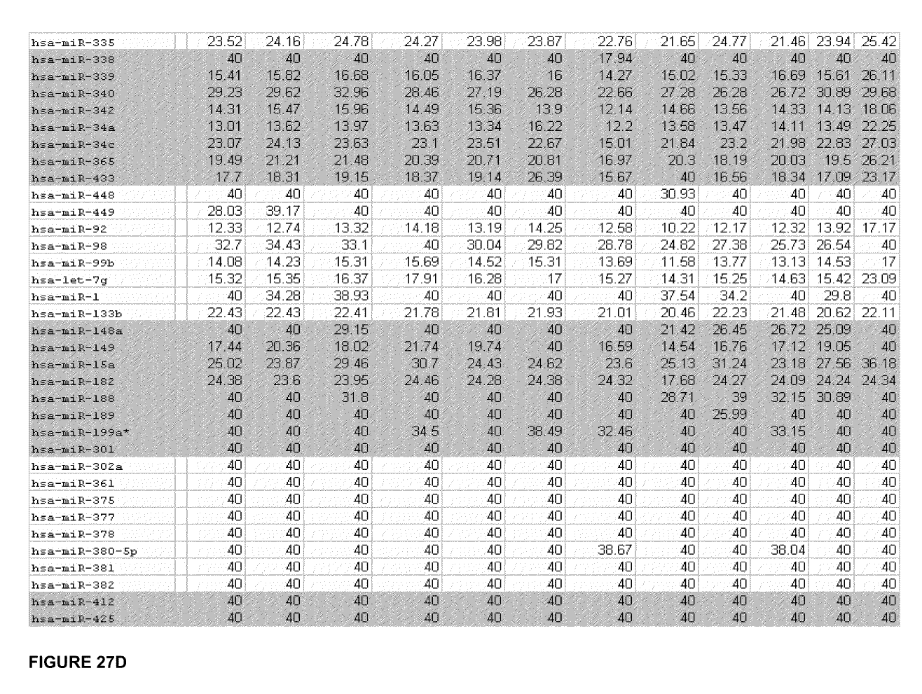

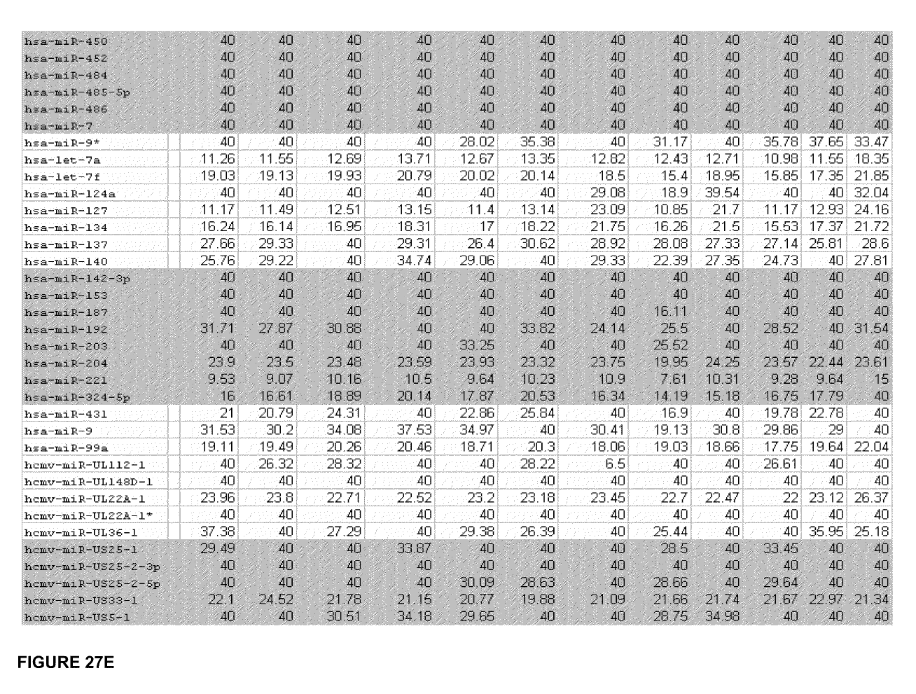

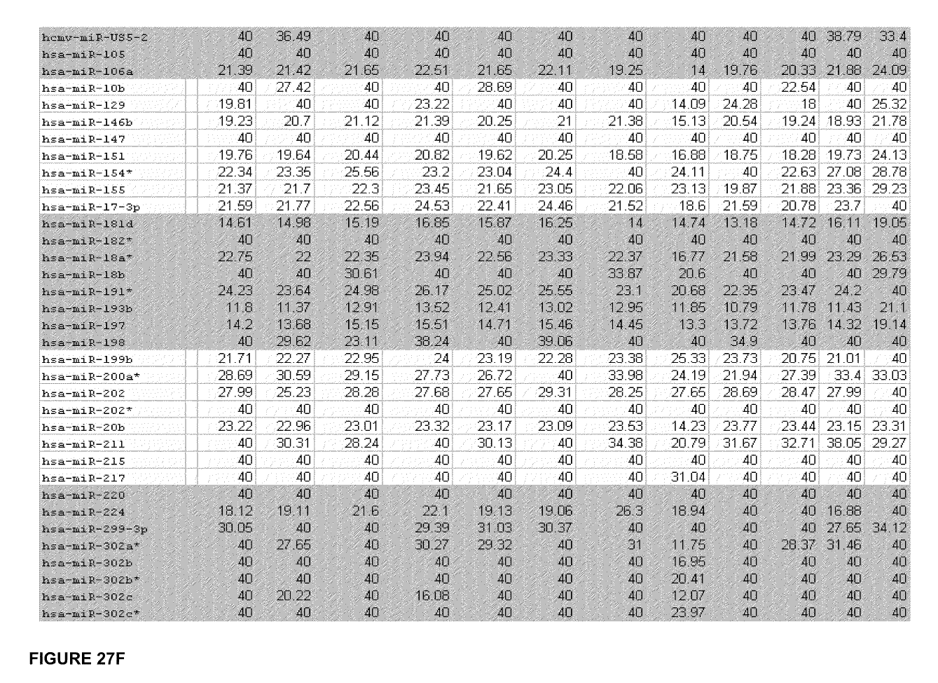

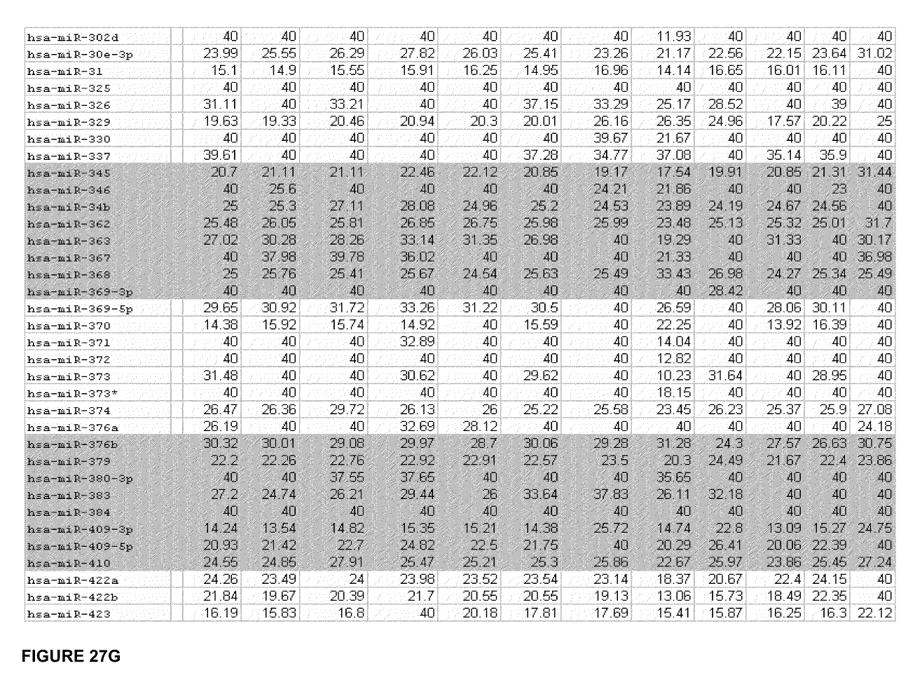

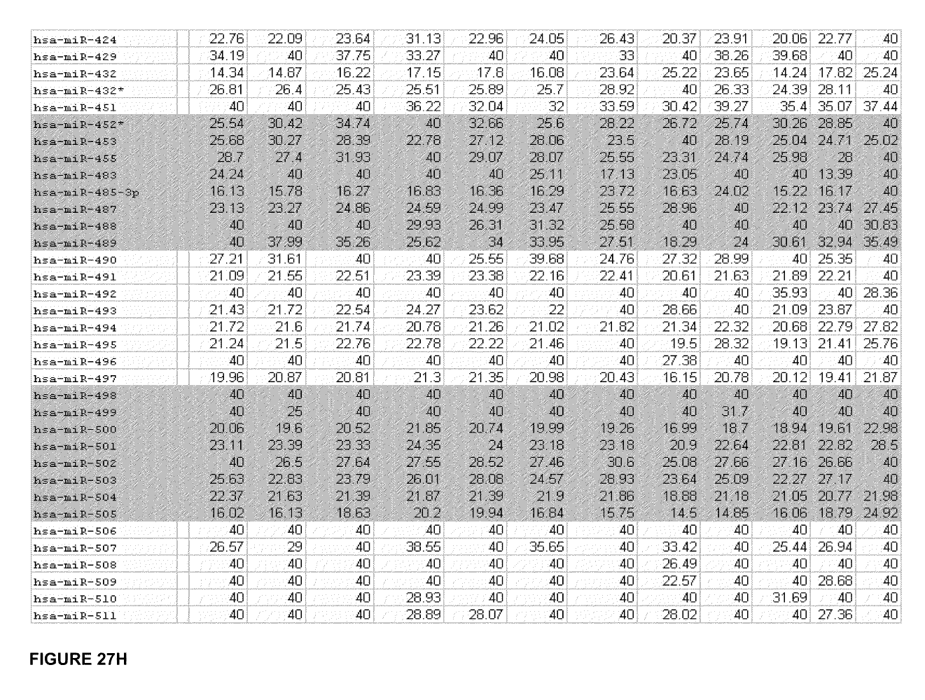

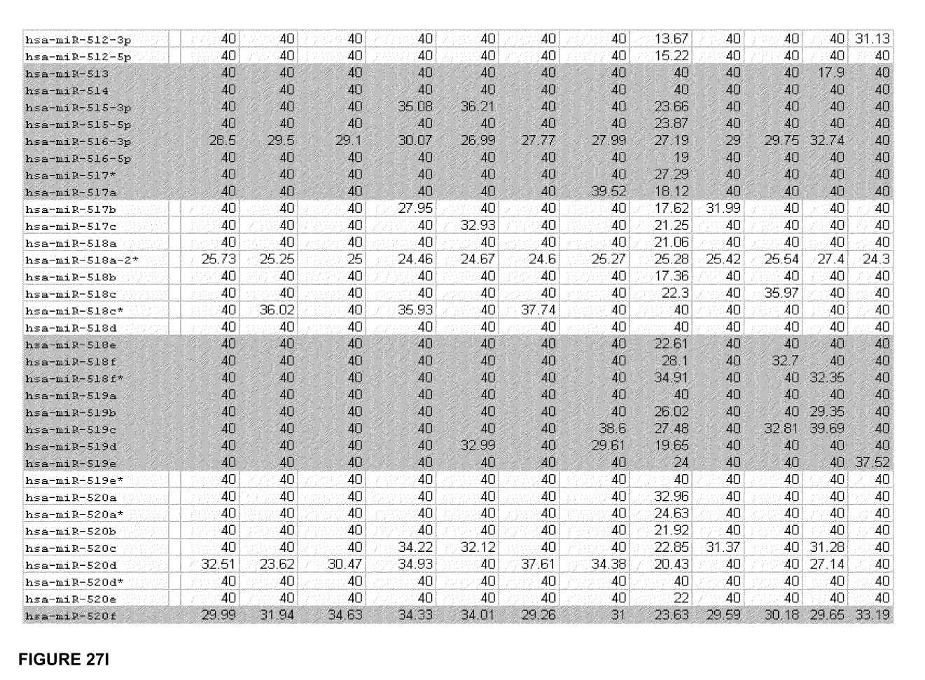

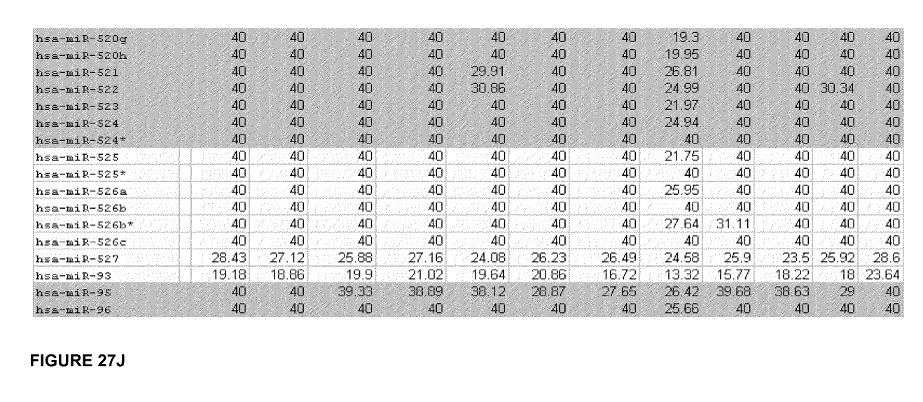

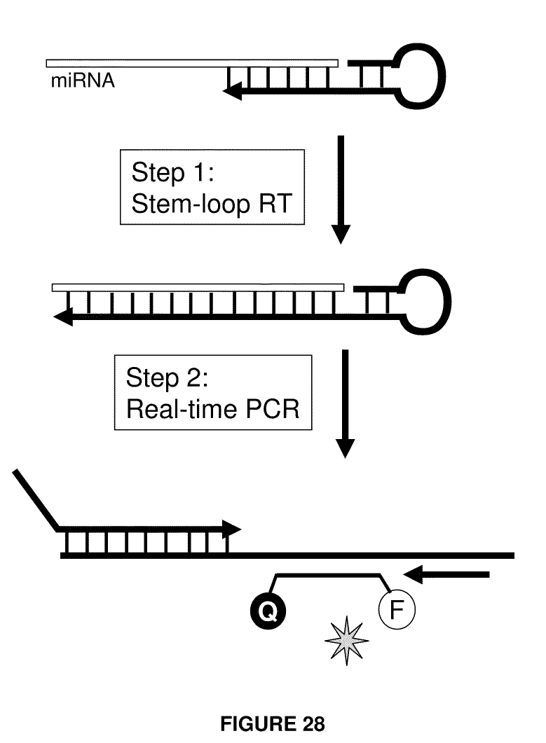

A hierarchical clustering analysis can then be performed as is well known in the art. These software programs perform a hierarchical cluster analysis using a group of dissimilarities for the number of objects being clustered. At first, each object is put in its own cluster, then iteratively, each similar cluster is joined until there is one cluster. Distances between clusters are computed by Lance-Williams dissimilarity update formula (Becker, R. A., Chambers, J. M. and Wilks, A. R. (1988) The New S Language. Wadsworth & Brooks/Cole. (S version.); Everitt, B. (1974). Cluster Analysis. London: Heinemann Educ. Books). As an illustration, Example 29 describes colored dendrograms in FIGS. 27, 28a and 28b which show the global correlation of different clones. The vertical axis of the dendrograms displays the extent of similarity of the gene expression profiles of the cell clones. That is, the farther down they branch apart, the more similar they are. The vertical axis is a set of n−1 non-decreasing real values. The clustering height is the value of the criterion associated with the clustering method for the particular agglomeration. In order to determine if a new cell line is identical to existing cell lines, two types of replicates are performed: biological and technical replicates. Biological replicates require that new cell lines be grown, mRNA harvested, and then the analysis compared. Technical replicates, on the other hand, analyze the same RNA twice. A line cutoff is then drawn just above where the replicates branch such that cells branching below the cutoff line are considered the same cell type.

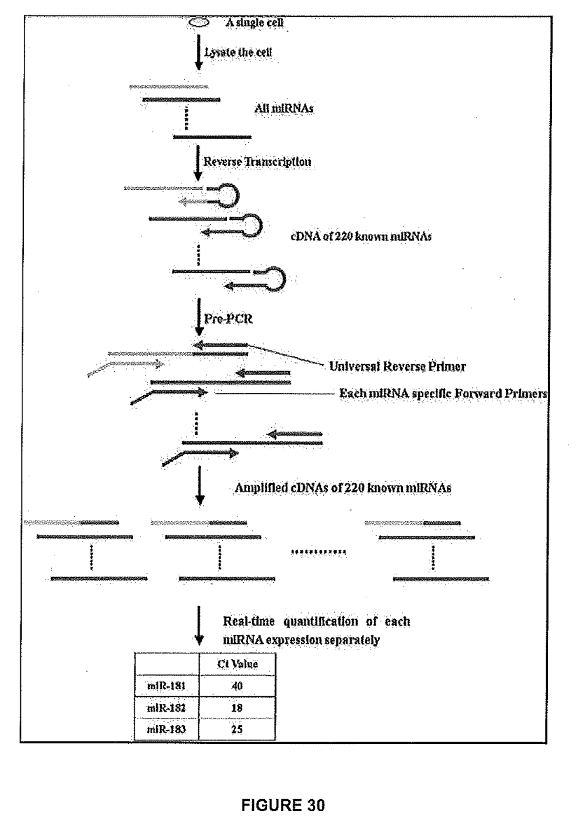

Another source of data for the database described above may be microRNA profiles of the single cell-derived and oligoclonal cell-derived cells generated according to the methods of the present invention. MicroRNAs (miRNA) are endogenous RNAs of ˜22 nucleotides that play important regulatory roles in animals & plants by targeting mRNAs for cleavage or translational repression. More than 700 miRNAs have been identified across species. Their expression levels vary among species and tissues. Low abundant miRNAs have been difficult to detect based on current technologies such as cloning, Northern hybridization, and the modified Invader® assay. In the present invention, an alternative approach using a new real-time quantitation method termed looped-primer RT-PCR was used for accurate and sensitive detection of miRNAs as well as other non-coding RNA (ncRNA) molecules present in human embryonic stem cells and in cell lines differentiated from human embryonic stem cells. As an illustration, FIG. 27 is a table displaying the microRNA profiles of eleven cell lines generated according to the methods of this invention (ACT 6-1, ACT 2-1, ACT B-11, ACT B-26, ACT B-3, ACT 2-2, ACTB-29, H9 Bio2, CM0-2, CM50-5 and Fb-p1). The NTC or no template control serves as the control for each of the amplified miRNAs. Another illustration is provided in Example 30 and FIG. 30, which describes the methodology of generating the microRNA profiles of human embryonic stem cells and differentiated progeny cells generated according to the methods of this invention.

In another embodiment of the invention, microRNA analysis may be used to identify the developmental pathways and cell types for in vitro differentiated hES cells. Dissected tissues are typically composed of many different cell populations, some of which have cellular activities characteristic of specialized tissue functions and other cells types providing support roles, for example, blood vessels and fibroblasts. Thus, gene expression analysis on whole tissues provides composite or average values for the levels of gene expression, which can obscure the gene expression profile for specialized individual cell types. On the other hand, microRNA expression analysis of single cells or a small number of cells from human or nonhuman embryonic or fetal tissues provides a means to generate a database of unique microRNA profiles for distinct populations of cells at different stages of differentiation. As described in Example 31, single cell analysis of microRNA expression may be determined as previously described by Tang, F., Hajkova, P., Barton, S. C., Lao, K., and Surani, M. A. (2006) MicroRNA expression profiling of single whole embryonic stem cells Nucleic Acids Res, 34, e9).

In another embodiment of the invention, gene expression analysis may be used to identify the developmental pathways and cell types for in vitro differentiated hES cells. Gene expression analysis of single cells or a small number of cells from human or nonhuman embryonic or fetal tissues provides another means to generate a database of unique gene expression profiles for distinct populations of cells at different stages of differentiation. As described in Example 32, gene expression analysis on single cells isolated from specific tissues may be performed as previously described by Kurimoto et al., Nucleic Acids Research (2006) Vol. 34, No. 5, e42.

Thus, cellular miRNA profiles on their own or in conjunction with gene expression profiles, immunocytochemistry, and proteomics provide molecular signatures that can be used to identify the tissue and developmental stage of differentiating cell lines.

This technique illustrates that the database may be used to accurately identify cell types and distinguish them from other cell types.

The cells of the present invention are also useful in providing a subset of gene expression markers that are expressed at relatively high levels in some cell lines while not be expressed at all in other cell lines as opposed to genes expressed in all cell lines but at different levels of expression. This subset of “all-or none” markers can be easily identified by comparing the levels of expression as measured for instance through the use of oligonucleotide probes or other means know in the art, and comparing the level of a gene's expression in one line compared to all the other lines of the present invention. Those genes that are expressed at relatively high levels in a subset of lines, and not at all in other lines, are used to generate a short list of gene expression markers. When applied to the cells and gene expression data described herein, where negative expression in Illumina 1 is <170 RFU and positive expression is >500 RFU, negative expression in Illumina 2 is <160 RFU and positive expression is >300 RFU, and negative expression in Affy is <50 RFU and positive expression is >250 RFU, a nonlimiting example of such genes is ACTC, AGC1, AKR1C1, ALDH1A1, ANXA8, APCDD1, AQP1, AREG, ATP8B4, BEX1, CFB, BMP4, C3, C6, C7, PRSS35, C20orf103, CCDC3, CD24, CDH3, CDH6, CLDN11, CNTNAP2, COL15A1, COL21A1, COMP, COP1, CRIP1, CRLF1, CRYAB, CXADR, DIO2, METTL7A, DKK2, DLK1, DPT, EGR2, EMID1, FGFR3, TMEM100, FMO1, FMO3, FOXF1, FOXF2, FST, GABRB1, GAP43, GDF5, GDF10, GJB2, GSC, HOXA5, HSD11B2, HSD17B2, HSPA6, HSPB3, HTRA3, ICAM5, ID4, IFI27, IFIT3, IGF2, IGFBP5, IL1R1, INA, KCNMB1, KIAA0644, KRT14, KRT17, KRT19, KRT34, LAMC2, TMEM119, IGFL3, LOC92196, MFAP5, MASP1, MEOX1, MEOX2, MGP, MMP1, MSX1, MSX2, MX1, MYBPH, MYH3, MYH11, MYL4, IL32, NLGN4X, TAGLN3, NPAS1, NPPB, OGN, OLR1, OSR2, PAX2, PAX9, PDE1A, PENK, PITX2, PODN, POSTN, PRELP, PRG4, PROM1, PRRX1, PRRX2, PTGS2, PTN, PTPRN, RARRES1, RASD1, RELN, RGMA, RGS1, RPS4Y2, S100A4, SERPINA3, SFRP2, SLITRK6, SMOC1, SMOC2, SNAP25, SOD3, SOX11, SRCRB4D, STMN2, SYT12, TAC1, TFPI2, RSPO3, THY1, TNFSF7, TNNT2, TRH, TSLP, TUBB4, UGT2B7, WISP2, ZD52F10, ZIC1, and ZIC2.

When applied to the identification of the cells of the present invention, cultured in the media in which they were expanded, and synchronized in quiescence as described in Example 29 at 18-21 doublings from the originally plated cell, and assayed using the microarray chips described herein, such markers are as shown in Table XX, below.

TABLE XX
Gene expression in exemplary progenitor cell lines

The group of cell lines X2.1, X2.2Rep1 and X2.2Rep2 are positive for the markers: CFB, CLDN11, COMP, CRLF1,
EGR2, FST, KRT14, KRT19, KRT34, MFAP5, MGP, PENK, PITX2, POSTN, PTGS2, RARRES1, S100A4, SOD3,
TFPI2, THY1 and ZIC1 and are negative for the markers: AGC1, ALDH1A1, APCDD1, AREG, ATP8B4, C6, C7,
C20orf103, CCDC3, CDH3, CDH6, CNTNAP2, COP1, CXADR, DIO2, METTL7A, DKK2, DLK1, EMID1, FGFR3,
FMO3, FOXF1, FOXF2, GABRB1, GDF10, GSC, HSD11B2, HSD17B2, HSPA6, HSPB3, ID4, IGF2, IGFBP5, INA,
KCNMB1, IGFL3, LOC92196, MEOX1, MSX2, MX1, MYBPH, MYH11, MYL4, NLGN4X, NPPB, PAX2, PAX9,
PDE1A, PRELP, PROM1, RASD1, RELN, RGS1, RPS4Y2, SFRP2, SMOC1, SMOC2, SNAP25, SYT12, TAC1,
RSPO3, TUBB4, UGT2B7, WISP2, ZD52F10 and ZIC2.
The cell line B1 is positive for the markers: CD24, CDH6, HTRA3, INA, KRT17, KRT19, LAMC2, MMP1, IL32,
TAGLN3, PAX2, RELN, UGT2B7 and ZIC2 and is negative for the markers: ACTC, AGC1, ALDH1A1, APCDD1,
ATP8B4, BEX1, CFB, C3, C6, C7, PRSS35, C20orf103, CCDC3, CDH3, CNTNAP2, COL15A1, COL21A1, COP1,
CRLF1, DIO2, METTL7A, DKK2, DLK1, DPT, EGR2, EMID1, FGFR3, TMEM100, FMO1, FMO3, FOXF1, FOXF2,
FST, GABRB1, GAP43, GDF10, GSC, HOXA5, HSD11B2, HSD17B2, HSPA6, ID4, IFI27, IGF2, KCNMB1,
KIAA0644, KRT14, TMEM119, IGFL3, LOC92196, MFAP5, MASP1, MEOX2, MGP, MYBPH, MYH3, MYH11,
MYL4, NPAS1, OGN, OLR1, OSR2, PAX9, PDE1A, PENK, POSTN, PRELP, PRG4, PROM1, PRRX1, PRRX2, PTN,
PTPRN, RARRES1, RASD1, RGMA, RGS1, SERPINA3, SLITRK6, SMOC1, SMOC2, SNAP25, SOD3, STMN2,
TAC1, RSPO3, TNNT2, TRH, TSLP, TUBB4, WISP2 and ZIC1.
The group of cell lines X4.1, X4.3 and B10 are positive for the markers: MMP1, AQP1, CDH6, HTRA3, INA, KRT19,
LAMC2, IL32, TAGLN3, NPPB and UGT2B7 and are negative for the markers: AGC1, ALDH1A1, APCDD1, AREG,
ATP8B4, CFB, C3, C6, C7, C20orf103, CNTNAP2, COL21A1, COMP, COP1, CRLF1, DIO2, METTL7A, DKK2,
DLK1, DPT, EMID1, TMEM100, FMO1, FMO3, FOXF1, FOXF2, GABRB1, GAP43, GSC, HOXA5, HSD11B2,
HSD17B2, HSPA6, ID4, IFI27, IFIT3, IGF2, KRT14, TMEM119, LOC92196, MASP1, MEOX2, MGP, MYBPH,
MYH3, MYL4, OGN, OSR2, PAX9, PDE1A, PENK, PRELP, PRRX2, PTN, RARRES1, RGMA, RGS1, RPS4Y2,
SERPINA3, SLITRK6, SMOC1, SMOC2, TAC1, RSPO3, TNNT2, TRH, TUBB4 and WISP2.
The group of cell lines B11, B25, B26 and B3 are positive for the markers: AKR1C1, CFB, BMP4, CLDN11, FST,
GDF5, HTRA3, IL1R1, KRTI4, KRT19, KRT34, MGP, MMP1, PODN, POSTN, PRG4, RARRES1, S100A4, THY1
and ZIC1 and are negative for the markers: ACTC, ALDH1A1, APCDD1, C6, C7, C20orf103, CCDC3, CD24, CXADR,
DIO2, DKK2, DLK1, EMID1, FGFR3, FMO1, FMO3, FOXF1, FOXF2, GABRB1, GDF10, HSD11B2, HSD17B2,
HSPA6, HSPB3, ID4, IGF2, INA, KCNMB1, IGFL3, LOC92196, MEOX1, MSX1, MYBPH, MYH3, MYH11, MYL4,
NLGN4X, TAGLN3, NPPB, OLR1, PAX2, PAX9, PROM1, RASD1, RGS1, RPS4Y2, SLITRK6, SMOC1, SMOC2,
SNAP25, TAC1, RSPO3, TUBB4, UGT2B7, ZD52F10 and ZIC2.
The group of cell lines B12 and B4 are positive for the markers: CLDN11, FST, GDF5, HTRA3, KRT19, KRT34,
MFAP5, MGP, MMP1, POSTN, PTGS2, S100A4, THY1 and ZIC1 and are negative for the markers: AGC1, ALDH1A1,
APCDD1, AREG, ATP8B4, C3, C6, C7, C20orf103, CCDC3, CDH3, CNTNAP2, COP1, CXADR, DIO2, DKK2,
DLK1, DPT, EMID1, FMO1, FMO3, FOXF1, FOXF2, GABRB1, GDF10, HOXA5, HSD11B2, HSD17B2, HSPA6,
HSPB3, IGFBP5, IGFL3, LOC92196, MEOX1, MYBPH, MYH3, MYH11, MYL4, NPAS1, NPPB, OLR1, PAX2,
PAX9, PITX2, PROM1, RGS1, SLITRK6, SMOC1, SMOC2, SNAP25, TAC1, RSPO3, TNNT2, TRH, TUBB4,
ZD52F10 and ZIC2.
The group of cell lines B20 and B15 are positive for the markers: BMP4, CD24, CRIP1, HTRA3, KRT19, LAMC2,
MGP, MMP1, POSTN, RELN, S100A4, THY1 and UGT2B7 and are negative for the markers: AGC1, ALDH1A1,
ANXA8, AREG, ATP8B4, CFB, C6, C7, C20orf103, CNTNAP2, DIO2, METTL7A, DLK1, DPT, EMID1, TMEM100,
FMO1, FMO3, FOXF2, GABRB1, GSC, HOXA5, HSD11B2, HSD17B2, HSPA6, ID4, IFI27, KRT14, KRT34, IGFL3,
MASP1, MEOX1, MEOX2, MYBPH, MYH3, MYL4, NPAS1, NPPB, OGN, OLR1, OSR2, PAX9, PDE1A, PENK,
PROM1, PRRX2, RGS1, SLITRK6, SMOC1, SMOC2, STMN2, TAC1, TNNT2, TRH, TUBB4, WISP2 and ZIC1.
The group of cell lines B16Bio1b, B16Bio2b, E72 and E75 are positive for the markers: AKR1C1, BMP4, CLDN11,
FST, GDF5, HTRA3, IL1R1, KRT19, KRT34, MFAP5, MGP, MMP1, OSR2, PODN, POSTN, PRG4, PRRX1,
RARRES1, S100A4, SOD3, THY1 and ZIC1 and are negative for the markers: ACTC, AGC1, ALDH1A1, AREG, C6,
C7, C20orf103, CCDC3, CDH3, CNTNAP2, DKK2, EMID1, FGFR3, FMO3, FOXF1, FOXF2, GABRB1, GDF10,
HSD11B2, HSD17B2, HSPA6, ID4, IGF2, INA, LAMC2, IGFL3, LOC92196, MEOX1, MSX1, MYBPH, MYH11,
MYL4, NLGN4X, NPAS1, NPPB, OLR1, PAX2, PAX9, PROM1, PTPRN, RASD1, RGS1, SLITRK6, SMOC1,
SMOC2, SNAP25, TAC1, RSPO3, TNNT2, TUBB4, ZD52F10 and ZIC2.
The group of cell lines B17Bio1b, B17Bio2c and B17Bio3c are positive for the markers: BEX1, COL15A1, CRIP1,
CRYAB, HTRA3, KCNMB1, KRT19, MGP, POSTN, S100A4, SFRP2, THY1 and TNFSF7 and are negative for the
markers:, AGC1, ALDH1A1, APCDD1, AREG, ATP8B4, C6, C7, CNTNAP2, METTL7A, DLK1, DPT, EMID1,
FMO1, FMO3, FOXF1, GABRB1, GSC, HOXA5, HSD11B2, HSD17B2, HSPA6, IFI27, KRT14, KRT34, IGFL3,
MASP1, MEOX1, MEOX2, MYBPH, MYH3, MYL4, NPPB, OGN, PAX9, PDE1A, PENK, PROM1, RASD1, RGS1,
SLITRK6, SMOC1, SMOC2, STMN2, TAC1, TRH, TSLP, TUBB4 and ZIC1.
The group of cell lines B2, B7 and X6.1 are positive for the markers: AKR1C1, CFB, BMP4, C3, CLDN11, COL21A1,
FST, GDF5, HTRA3, ICAM5, IL1R1, KRT19, MGP, MMP1, PENK, PODN, POSTN, PRG4, RARRES1, RGMA,
S100A4, SERPINA3, SOD3, STMN2, THY1 and WISP2 and are negative for the markers: ACTC, AGC1, ALDH1A1,
C6, C7, C20orf103, CCDC3, CD24, CDH3, CXADR, DIO2, DLK1, EMID1, FGFR3, FMO3, FOXF1, FOXF2,
GABRB1, GDF10, HSD11B2, HSD17B2, HSPA6, HSPB3, ID4, IGF2, INA, IGFL3, LOC92196, MEOX1, MYH11,
MYL4, NLGN4X, TAGLN3, NPAS1, NPPB, OLR1, PAX2, PAX9, PITX2, PROM1, PTPRN, RASD1, RGS1, RPS4Y2,
SLITRK6, SMOC1, SMOC2, SNAP25, SOX11, TAC1, RSPO3, TUBB4, UGT2B7, ZD52F10 and ZIC2.
The group of cell lines B22, CM30.2 and X6 are positive for the markers: BMP4, CLDN11, CRIP1, CRYAB, HTRA3,
KRT19, S100A4, SFRP2, SRCRB4D, THY1 and UGT2B7 and are negative for the markers: AGC1, ALDH1A1,
APCDD1, AREG, ATP8B4, C3, C6, C7, C20orf103, CDH3, CNTNAP2, COL21A1, COP1, DIO2, METTL7A, DKK2,
DLK1, DPT, FMO1, FMO3, FOXF1, FOXF2, GABRB1, GSC, HOXA5, HSD11B2, HSPA6, IFI27, IFIT3, IGF2,
KRT14, MASP1, MEOX2, MYBPH, MYH3, MYH11, NPPB, OGN, OLR1, OSR2, PAX9, PDE1A, PENK, PROM1,
RGS1, SMOC1, SNAP25, STMN2, TAC1, TRH, TSLP, TUBB4 and WISP2.
The group of cell lines B27, B9, CM10.1, X2, X4.2 and X4.4 are positive for the markers: HTRA3, KRT19, LAMC2,
IL32, TAGLN3, PAX2, RELN and UGT2B7 and are negative for the markers: AGC1, ALDH1A1, APCDD1, AREG,
ATP8B4, CFB, C3, C6, C7, C20orf103, CCDC3, CDH3, CNTNAP2, COL21A1, COP1, CRLF1, DIO2, METTL7A,
DLK1, DPT, EMID1, TMEM100, FMO1, FMO3, FOXF1, FOXF2, GABRB1, GAP43, GSC, HOXA5, HSD11B2,
HSD17B2, HSPA6, IFI27, IGF2, KIAA0644, KRT14, IGFL3, LOC92196, MASP1, MEOX2, MGP, MYH3, MYH11,
MYL4, NPAS1, OGN, OLR1, OSR2, PAX9, PDE1A, PENK, PRELP, PTN, RARRES1, RGMA, RGS1, SERPINA3,
SLITRK6, SMOC1, SMOC2, SNAP25, SOD3, STMN2, TAC1, RSPO3, TNNT2, TRH, TUBB4 and WISP2.
The cell line B28 is positive for the markers: CFB, BMP4, COL15A1, CRIP1, CRYAB, FST, GAP43, IL1R1, KCNMB1,
KRT14, KRT19, KRT34, MFAP5, MGP, MMP1, IL32, PODN, POSTN, S100A4, THY1 and ZIC1 and are negative for
the markers: ACTC, ALDH1A1, ANXA8, AREG, ATP8B4, BEX1, C3, C6, C7, C20orf103, CCDC3, CNTNAP2,
CXADR, DIO2, METTL7A, DKK2, DLK1, EMID1, FGFR3, FMO1, FMO3, FOXF1, FOXF2, GABRB1, GDF10,
HOXA5, HSD11B2, HSD17B2, HSPA6, ID4, IFI27, IGF2, IGFBP5, INA, IGFL3, LOC92196, MASP1, MEOX1,
MYBPH, MYH3, MYL4, NLGN4X, NPAS1, NPPB, OLR1, PAX9, PDE1A, PITX2, PROM1, PTPRN, RASD1, RGS1,
RPS4Y2, SLITRK6, SMOC1, SMOC2, SNAP25, STMN2, TAC1, TRH, TSLP, TUBB4, ZD52F10 and ZIC2.
The cell line B29 is positive for the markers: ANXA8, AQP1, CD24, CDH6, CRIP1, GJB2, HTRA3, KRT17, KRT19,
LAMC2, IL32, TAGLN3, PAX2, RELN, S100A4, SFRP2, SRCRB4D, THY1, TNFSF7, UGT2B7, ZD52F10 and ZIC2
and are negative for the markers: AGC1, ALDH1A1, APCDD1, AREG, ATP8B4, BEX1, C3, C6, C7, C20orf103,
CCDC3, CLDN11, CNTNAP2, COL21A1, COP1, CRLF1, DIO2, METTL7A, DLK1, DPT, EMID1, TMEM100, FMO1,
FMO3, FOXF1, FOXF2, GABRB1, GAP43, GDF10, GSC, HOXA5, HSD11B2, HSD17B2, HSPA6, HSPB3, IFI27,
IFIT3, IGF2, KRT14, KRT34, IGFL3, MFAP5, MASP1, MEOX2, MMP1, MSX1, MYBPH, MYH3, MYL4, NPAS1,
NPPB, OGN, OLR1, OSR2, PAX9, PDE1A, PENK, PITX2, POSTN, PRG4, PROM1, PRRX2, PTPRN, RARRES1,
RASD1, RGS1, RPS4Y2, SERPINA3, SLITRK6, SMOC1, SMOC2, SNAP25, SOD3, STMN2, TAC1, RSPO3, TRH,
TSLP, TUBB4, WISP2 and ZIC1.
The cell line B30 is positive for the markers: PRSS35, CDH6, COL21A1, CRIP1, CRYAB, DKK2, GAP43, KCNMB1,
KRT17, KRT19, PRRX1, PTN, RGMA, S100A4, SOX11 and ZIC2 and are negative for the markers: ACTC, AGC1,
AKR1C1, ALDH1A1, ANXA8, APCDD1, AQP1, AREG, ATP8B4, CFB, C3, C6, C7, C20orf103, CD24, CDH3,
CLDN11, CNTNAP2, COL15A1, COMP, COP1, CRLF1, METTL7A, DPT, EGR2, EMID1, TMEM100, FMO1, FMO3,
FOXF1, FOXF2, GABRB1, GDF10, GJB2, GSC, HOXA5, HSD11B2, HSD17B2, HSPA6, HSPB3, IFI27, IFIT3, IGF2,
KRT34, LAMC2, IGFL3, LOC92196, MFAP5, MASP1, MEOX1, MEOX2, MSX1, MYBPH, MYH3, MYL4,
NLGN4X, NPPB, OGN, OLR1, PAX2, PAX9, PDE1A, PENK, PITX2, PRG4, PROM1, PTPRN, RARRES1, RASD1,
RELN, RGS1, RPS4Y2, SFRP2, SLITRK6, SMOC1, SNAP25, STMN2, TAC1, TFPI2, TNFSF7, TNNT2, TRH, TSLP,
TUBB4, UGT2B7, WISP2, ZD52F10 and ZIC1.
The cell line B6 is positive for the markers: CCDC3, CDH6, COL15A1, CRIP1, DKK2, FST, GDF10, HTRA3, KRT19,
LOC92196, MYL4, NLGN4X, S100A4, SOX11, SRCRB4D, THY1, ZIC1 and ZIC2 and are negative for the markers:
AGC1, AKR1C1, ALDH1A1, AREG, ATP8B4, BEX1, CFB, C3, C6, C7, CNTNAP2, COMP, COP1, DIO2, METTL7A,
DLK1, DPT, EMID1, TMEM100, FMO3, FOXF1, FOXF2, GABRB1, GSC, HOXA5, HSD11B2, HSPA6, HSPB3, ID4,
IFI27, IFIT3, KRT14, TMEM119, MFAP5, MASP1, MEOX1, MEOX2, MGP, MMP1, MSX2, MYBPH, MYH3,
NPAS1, NPPB, OGN, OLR1, OSR2, PAX2, PAX2, PAX9, PDE1A, PENK, PRG4, PROM1, PTPRN, RASD1, RGS1, RPS4Y2,
SLITRK6, SMOC1, SNAP25, STMN2, TAC1, TRH, TSLP, TUBB4, UGT2B7, WISP2 and ZD52F10.
The cell line C4ELS5.1 is positive for he markers: AKR1C1, C7, CDH6, COL15A1, DIO2, FMO1, FMO3, FOXF2,
IGF2, IL1R1, KRT19, LAMC2, TMEM119, PODN, PRRX1, PRRX2, RGMA, SFRP2, TAC1, TFPI2 and RSPO3 and
are negative for the markers: ACTC, AGC1, ALDH1A1, ANXA8, APCDD1, AQP1, AREG, ATP8B4, BEX1, CFB,
BMP4, C3, C20orf103, CCDC3, CDH3, CLDN11, CNTNAP2, COMP, COP1, CRLF1, CRYAB, CXADR, DKK2,
DLK1, EGR2, EMID1, FGFR3, FOXF1, GABRB1, GAP43, GDF10, GJB2, HOXA5, HSD17B2, HSPA6, HSPB3,
ICAM5, ID4, IFI27, KRT14, KRT17, KRT34, IGFL3, LOC92196, MFAP5, MEOX1, MEOX2, MGP, MMP1, MSX1,
MSX2, MX1, MYBPH, MYH3, MYH11, MYL4, IL32, NLGN4X, TAGLN3, NPAS1, NPPB, OLR1, PAX2, PAX9,
PENK, PITX2, POSTN, PRELP, PROM1, PTPRN, RARRES1, RELN, RGS1, RPS4Y2, SMOC1, SMOC2, STMN2,
THY1, TNFSF7, TNNT2, TRH, TUBB4, UGT2B7, ZD52F10, ZIC1 and ZIC2.
The cell line C4ELS5.5 is positive for the markers: BEX1, BMP4, C7, PRSS35, CDH6, DKK2, FMO3, FOXF2, FST,
GDF10, HSD17B2, IGF2, TMEM119, PITX2, PODN, PRRX1, SERPINA3, SFRP2, TFPI2 and ZIC2 and are negative
for the markers: AGC1, ALDH1A1, APCDD1, AQP1, AREG, ATP8B4, C3, C6, C20orf103, CD24, CDH3, CNTNAP2,
COMP, COP1, CRLF1, CXADR, DLK1, DPT, EMID1, FGFR3, TMEM100, FOXF1, GJB2, HOXA5, HSD11B2,
HSPA6, HSPB3, ID4, IFI27, KCNMB1, KRT14, KRT17, KRT34, IGFL3, MFAP5, MEOX1, MEOX2, MGP, MMP1,
MSX2, MX1, MYBPH, MYH3, MYH11, IL32, NLGN4X, TAGLN3, NPPB, OGN, OLR1, OSR2, PAX2, PAX9,
PDE1A, PENK, PRELP, PRG4, PTPRN, RARRES1, RASD1, RELN, RGS1, SMOC2, STMN2, TAC1, THY1, TNFSF7,
TNNT2, TRH, TSLP, TUBB4, WISP2, ZD52F10 and ZIC1.
The cell line C4ELSR.12 is positive for the markers: C7, CDH6, COL21A1, DIO2, FMO1, FMO3, FOXF2, FST, IGF2,
IL1R1, TMEM119, PRRX1, PRRX2, PTN, RGMA, SFRP2, SRCRB4D, TAC1, TFPI2, RSPO3, UGT2B7 and ZIC2 and
are negative for the markers: ACTC, ACC1, ALDH1A1, ANXA8, APCDD1, AQP1, ATP8B4, C3, C20orf103, CD24,
CDH3, CNTNAP2, COMP, COP1, CRLF1, CXADR, DPT, EMID1, FGFR3, TMEM100, FOXF1, GABRB1, GAP43,
GJB2, HOXA5, HSPA6, HSPB3, ICAM5, IFI27, INA, KRT14, KRT17, KRT34, IGFL3, MFAP5, MEOX1, MEOX2,
MGP, MMP1, MX1, MYBPH, MYH11, MYL4, IL32, NLGN4X, NPAS1, NPPB, OLR1, OSR2, PAX2, PAX9, PENK,
POSTN, PRELP, PROM1, PTPRN, RARRES1, RASD1, RELN, RGS1, SLITRK6, SMOC2, STMN2, SYT12, THY1,
TNFSF7, TNNT2, TRH, TSLP, TUBB4, WISP2, ZD52F10 and ZIC1.
The group of cell lines C4ELSR2, C4ELSR2Bio2 and C4ELSR2Bio2.1 are positive for the markers: C7, CDH6,
COL21A1, DKK2, FMO3, FST, GSC, IGF2, TMEM119, PITX2, SFRP2, TFPI2 and ZIC2 and are negative for the
markers: ACTC, AGC1, ALDH1A1, APCDD1, AQP1, ATP8B4, CFB, C3, C6, CCDC3, CD24, CDH3, CLDN11,
CNTNAP2, COMP, COP1, CRLF1, CRYAB, DLK1, DPT, EMID1, FGFR3, TMEM100, FOXF1, GABRB1, GJB2,
HOXA5, HSD11B2, HSD17B2, HSPA6, HSPB3, ID4, IFI27, KIAA0644, KRT14, KRT17, KRT34, IGFL3, MFAP5,
MEOX1, MGP, MSX2, MX1, MYBPH, MYH3, MYH11, IL32, NLGN4X, NPAS1, NPPB, OLR1, PAX2, PAX9,
PDE1A, PENK, POSTN, PRELP, PROM1, PTPRN, RARRES1, RASD1, RELN, RGS1, SMOC1, SMOC2, STMN2,
THY1, TNFSF7, TRH, TSLP, TUBB4, ZD52F10 and ZIC1.
The group of cell lines CMO.2 and E31 are positive for the markers: AQP1, CD24, CDH6, HTRA3, KRT19, KRT34,
TAGLN3, RELN, S100A4, SFRP2, SRCRB4D and UGT2B7 and are negative for the markers: AGC1, ALDH1A1,
APCDD1, AREG, ATP8B4, CFB, C3, C6, C7, C20orf103, CDH3, CNTNAP2, COMP, COP1, CRLF1, DIO2,
METTL7A, DLK1, DPT, EMID1, TMEM100, FMO1, FMO3, FOXF1, FOXF2, GABRB1, GAP43, GSC, HOXA5,
HSD11B2, HSPA6, HSPB3, IFI27, IFIT3, IGF2, KRT14, MFAP5, MASP1, MEOX2, MYH3, NPAS1, OGN, OLR1,
OSR2, PAX9, PDE1A, PENK, PRG4, PROM1, PTPRN, RARRES1, RASD1, RGS1, SERPINA3, SLITRK6, SMOC1,
SMOC2, SNAP25, SOD3, STMN2, TAC1, TRH, TSLP, TUBB4 and WISP2.
The group of cell lines CMO.2, CMO.5 and CM50.5 are positive for the markers: PRSS35, CLDN11, CRIP1, CRYAB,
FST, KRT19, KRT34, MFAP5, MEOX2, MGP, MMP1, PODN, POSTN, PRRX1, S100A4, THY1 and ZIC1 and are
negative for the markers: ACTC, ALDH1A1, APCDD1, AREG, ATP8B4, BEX1, C3, C6, C7, C20orf103, CCDC3,
CDH3, CNTNAP2, CXADR, DIO2, DKK2, DLK1, EMID1, TMEM100, FMO1, FMO3, FOXF1, FOXF2, GABRB1,
GDF10, GJB2, GSC, HSD11B2, HSD17B2, HSPA6, IGF2, IGFBP5, INA, LAMC2, IGFL3, LOC92196, MEOX1, MX1,
MYBPH, MYL4, NLGN4X, TAGLN3, NPAS1, NPPB, PAX2, PAX9, PDE1A, PENK, PITX2, PROM1, PTPRN,
RASD1, RGS1, SLITRK6, SMOC1, SMOC2, SNAP25, STMN2, TAC1, RSPO3, TRH, TSLP, TUBB4, ZD52F10 and
ZIC2.
The group of cell lines CM10.4, CM20.4, CM30.5 and X2.3 are positive for the markers: CLDN11, COMP, CRIP1, FST,
KRT19, KRT34, MFAP5, MGP, PITX2, POSTN, S100A4 and THY1 and are negative for the markers: ACTC,
ALDH1A1, AQP1, ATP8B4, C6, C7, C20orf103, CCDC3, CDH3, CNTNAP2, COP1, CXADR, METTL7A, DLK1,
DPT, EMID1, FGFR3, TMEM100, FMO1, FMO3, FOXF1, FOXF2, GABRB1, GDF10, HSD11B2, HSD17B2, HSPA6,
HSPB3, IGF2, IGFL3, LOC92196, MEOX1, MX1, MYBPH, MYH3, MYH11, MYL4, NLGN4X, TAGLN3, NPPB,
PAX2, PAX9, PDE1A, PRELP, PROM1, PTPRN, RASD1, RELN, RGS1, SLITRK6, SMOC2, SNAP25, STMN2,
TAC1, RSPO3, TUBB4, UGT2B7, WISP2, ZD52F10 and ZIC2.
The group of cell lines E111 and E111Bio2 are positive for the markers: CD24, CDH6, CRIP1, HTRA3, INA, TAGLN3,
SFRP2, SRCRB4D, UGT2B7 and ZIC2 and are negative for the markers: AGC1, AKR1C1, ALDH1A1, APCDD1,
AREG, ATP8B4, CFB, C3, C6, C7, C20orf103, CDH3, CNTNAP2, COP1, CRLF1, DIO2, METTL7A, DLK1, DPT,
EMID1, TMEM100, FMO1, FMO3, FOXF1, FOXF2, GABRB1, GAP43, GSC, HOXA5, HSD11B2, HSD17B2, HSPA6,
HSPB3, ID4, IFI27, IFIT3, IGF2, KRT14, LAMC2, MASP1, MEOX2, MX1, MYBPH, MYH3, MYH11, NPAS1, OGN,
OLR1, PAX9, PDE1A, PENK, PRG4, PROM1, PRRX2, PTPRN, RARRES1, RASD1, RGMA, RGS1, SLITRK6,
SMOC1, SMOC2, SNAP25, STMN2, TAC1, TNNT2, TRH, TUBB4 and WISP2.
The cell line E120 is positive for the markers: ACTC, BEX1, CLDN11, COL15A1, CRIP1, CRYAB, FST, GDF10,
GJB2, HTRA3, IGFL3, MGP, MX1, IL32, POSTN, S100A4, SERP2, THY1, TNFSF7, ZD52F10 and ZIC2 and are
negative for the markers: AGC1, AKR1C1, ALDH1A1, APCDD1, AQP1, AREG, ATP8B4, BMP4, C3, C6, C7,
PRSS35, C20orf103, CD24, CDH3, CNTNAP2, COL21A1, COMP, COP1, CRLF1, CXADR, DIO2, METTL7A, DKK2,
DLK1, EMID1, FGFR3, FMO1, FMO3, FOXF1, FOXF2, GABRB1, GAP43, GDF5, GSC, HOXA5, HSD11B2,
HSD17B2, HSPA6, HSPB3, IFI27, IGF2, INA, KRT14, LAMC2, TMEM119, MASP1, MEOX2, MMP1, MSX2,
MYBPH, MYH3, MYH11, NLGN4X, TAGLN3, NPAS1, NPPB, OGN, OLR1, OSR2, PAX2, PAX9, PDE1A, PENK,
PITX2, PODN, PRG4, PROM1, RASD1, RELN, RGMA, RGS1, SLITRK6, SMOC1, SMOC2, SNAP25, STMN2,
SYT12, TAC1, RSPO3, TNNT2, TRH, TUBB4, UGT2B7 and WISP2.
The cell line E15 is positive for the markers: ACTC, BEX1, PRSS35, CRIP1, CRYAB, GAP43, GDF5, HTRA3, KRT19,
MGP, MMP1, POSTN, PRRX1, S100A4, SOX11, SRCRB4D and THY1 and are negative for the markers: AGC1,
AKR1C1, ALDH1A1, ANXA8, APCDD1, AQP1, AREG, ATP8B4, CFB, C3, C6, C7, C20orf103, CDH3, CNTNAP2,
COP1, CXADR, METTL7A, DLK1, DPT, EGR2, EMID1, TMEM100, FMO1, FMO3, FOXF1, FOXF2, GABRB1,
GDF10, GJB2, GSC, HOXA5, HSD11B2, HSD17B2, HSPA6, HSPB3, IFI27, IFIT3, IGF2, INA, KRT14, TMEM119,
IGFL3, LOC92196, MFAP5, MASP1, MEOX1, MEOX2, MSX1, MX1, MYBPH, MYH3, MYL4, NLGN4X, TAGLN3,
NPAS1, NPPB, OGN, OLR1, PAX2, PAX9, PDE1A, PENK, PITX2, PRG4, PROM1, PTPRN, RARRES1, RASD1,
RELN, RGS1, SLITRK6, SMOC1, SMOC2, SNAP25, STMN2, TAC1, TFPI2, RSPO3, TNFSF7, TNNT2, TRH, TSLP,
TUBB4, UGT2B7, WISP2, ZD52F10 and ZIC1.
The cell line E164 is positive for the markers: AQP1, CD24, CDH6, CRIP1, HTRA3, KRT17, KRT19, IL32, TAGLN3,
PAX2, RELN, S100A4, SFRP2, SRCRB4D, THY1, TNFSF7, UGT2B7, ZD52F10 and ZIC2 and are negative for the
markers: ACTC, AGC1, ALDH1A1, ANXA8, APCDD1, AREG, ATP8B4, C3, C6, C7, C20orf103, CCDC3, CDH3,
CLDN11, CNTNAP2, COL15A1, COL21A1, COMP, COP1, CRLF1, DIO2, METTL7A, DKK2, DLK1, DPT, EGR2,
EMID1, TMEM100, FMO1, FMO3, FOXF1, FOXF2, GABRB1, GAP43, GDF5, GSC, HOXA5, HSD11B2, HSD17B2,
HSPA6, HSPB3, ID4, IFI27, KCNMB1, KRT14, KRT34, TMEM119, MFAP5, MASP1, MEOX2, MGP, MSX2,
MYBPH, MYH3, MYH11, MYL4, NPAS1, NPPB, OGN, OLR1, PAX9, PDE1A, PENK, PITX2, POSTN, PRELP,
PRG4, PRRX1, PRRX2, PTGS2, PTPRN, RARRES1, RASD1, RGMA, RGS1, SERPINA3, SLITRK6, SMOC1,
SMOC2, SNAP25, SOD3, STMN2, TAC1, TNNT2, TRH, TUBB4 and WISP2.
The group of cell lines E69 and E169 are positive for the markers: BEX1, CDH6, CRIP1, FST, GDF5, HTRA3, MMP1,
POSTN, PTN, S100A4 and ZIC2 and are negative for the markers: AGC1, ALDH1A1, APCDD1, AQP1, AREG,
ATP8B4, BMP4, C3, C6, C7, C20orf103, CDH3, CNTNAP2, COMP, CRLF1, CXADR, DLK1, DPT, EGR2, EMID1,
FMO1, FMO3, FOXF1, FOXF2, GABRB1, GJB2, GSC, HOXA5, HSD11B2, HSD17B2, HSPA6, HSPB3, IFI27, IGF2,
INA, KRT14, IGFL3, LOC92196, MASP1, MEOX1, MEOX2, MYBPH, MYH3, MYH11, MYL4, NLGN4X, TAGLN3,
NPAS1, NPPB, OGN, OLR1, PAX2, PAX9, PDE1A, PENK, PITX2, PROM1, RARRES1, RASD1, RELN, RGS1,
SLITRK6, SMOC1, SMOC2, SNAP25, STMN2, SYT12, TAC1, RSPO3, TNNT2, TRH, TUBB4, UGT2B7 and
ZD52F10.
The cell line E19 is positive for the markers: ACTC, BEX1, PRSS35, CLDN11, CRIP1, CRYAB, DKK2, HTRA3,
ICAM5, KRT17, KRT19, KRT34, MX1, POSTN, THY1, ZIC1 and ZIC2 and are negative for the markers: AGC1,
AKR1C1, ALDH1A1, APCDD1, AQP1, AREG, ATP8B4, CFB, BMP4, C3, C6, C7, C2orf103, CDH3, CNTNAP2,
COL21A1, COP1, CXADR, METTL7A, DLK1, DPT, EGR2, EMID1, TMEM100, FMO1, FMO3, FOXF1, FOXF2,
GABRB1, GAP43, GDF10, GJB2, GSC, HOXA5, HSD11B2, HSD17B2, HSPA6, IGF2, IL1R1, KIAA0644, TMEM119,
IGFL3, LOC92196, MASP1, MEOX1, MEOX2, MGP, MYBPH, MYH3, NLGN4X, TAGLN3, OGN, PAX2, PAX9,
PDE1A, PENK, PRG4, PROM1, PRRX2, RARRES1, RASD1, RELN, RGMA, RGS1, SFRP2, SLITRK6, SMOC1,
SMOC2, SNAP25, SOD3, STMN2, SYT12, TAC1, TFPI2, RSPO3, TNFSF7, TNNT2, TRH, TSLP, TUBB4, UGT2B7,
WISP2 and ZD52F10.
The group of cell lines E3, E30, E20Bio2, E67, E73, E57 and E84 are positive for the markers: KRT19, KRT34, MFAP5,
MGP, MMP1, S100A4, THY1 and ZIC1 and are negative for the markers: ALDH1A1, AREG, ATP8B4, C7, C20orf103,
CDH3, CNTNAP2, DKK2, DLK1, DPT, FMO1, FMO3, FOXF1, FOXF2, GDF10, GSC, HOXA5, HSD17B2, IGF2,
MEOX1, TAGLN3, NPPB, PAX9, PROM1, PTPRN, RGS1, SMOC1, SNAP25, STMN2, TAC1, TUBB4 and ZIC2.
The cell line E33 is positive for the markers: AQP1, PRSS35, CD24, CDH6, CLDN11, CRIP1, CRYAB, DKK2,
HTRA3, KRT17, KRT19, KRT34, LOC92196, MFAP5, MGP, MYH11, TAGLN3, POSTN, S100A4, SRCRB4D,
UGT2B7, ZIC1 and ZIC2 and are negative for the markers: AGC1, AKR1C1, ALDH1A1, APCDD1, AREG, ATP8B4,
CFB, C3, C6, C7, C20orf103, CDH3, CNTNAP2, COMP, COP1, CRLF1, DIO2, METTL7A, DLK1, DPT, EMID1,
TMEM100, FMO1, FMO3, FOXF1, FOXF2, GABRB1, GDF5, GJB2, GSC, HOXA5, HSD11B2, HSPA6, HSPB3,
IFI27, IFIT3, IGF2, TMEM119, IGFL3, MASP1, MX1, MYBPH, NPAS1, NPPB, OGN, OLR1, OSR2, PAX9, PDE1A,
PENK, PITX2, PRG4, PROM1, PTPRN, RARRES1, RASD1, RGMA, RGS1, SERPINA3, SFRP2, SLITRK6, SMOC1,
SMOC2, SNAP25, STMN2, TAC1, RSPO3, TRH, TSLP, TUBB4, WISP2 and ZD52F10.
The cell line E40 is positive for the markers: BEX1, CDH6, CLDN11, CRIP1, CRYAB, DKK2, FST, HTRA3, KRT17,
KRT19, MMP1, POSTN, S100A4, SRCRB4D and ZIC2 and are negative for the markers: AGC1, AKR1C1, ALDH1A1,
APCDD1, AQP1, AREG, ATP8B4, CFB, BMP4, C3, C6, C7, C20orf103, CDH3, CNTNAP2, COMP, COP1, CRLF1,
CXADR, METTL7A, DLK1, DPT, EGR2, EMID1, TMEM100, FMO1, FMO3, FOXF1, FOXF2, GABRB1, GJB2, GSC,
HOXA5, HSD11B2, HSD17B2, HSPA6, HSPB3, IFI27, IFIT3, IGF2, KIAA0644, KRT14, IGFL3, LOC92196, MASP1,
MEOX1, MEOX2, MGP, MX1, MYBPH, MYH3, NLGN4X, TAGLN3, NPAS1, NPPB, OGN, OLR1, OSR2, PAX2,
PAX9, PDE1A, PENK, PITX2, PRG4, PROM1, PRRX2, PTPRN, RARRES1, RASD1, RELN, RGS1, SLITRK6,
SMOC1, SMOC2, SNAP25, STMN2, SYT12, TAC1, TFPI2, RSPO3, TNFSF7, TNNT2, TRH, TSLP, TUBB4, WISP2,
ZD52F10 and ZIC1.
The cell line E44 is positive for the markers: BEX1, CLDN11, CRIP1, FST, GDF5, HTRA3, IFI27, IFIT3, MGP, MMP1,
MSX1, MX1, IL32, PRRX2, PTN, S100A4, SOD3 and ZIC2 and are negative for the markers: ACTC, AGC1,
ALDH1A1, AQP1, AREG, ATP8B4, BMP4, C6, C7, C20orf103, CDH3, CDH6, CNTNAP2, COL21A1, COMP,
CRLF1, DKK2, DPT, EGR2, EMID1, FGFR3, FMO1, FMO3, FOXF2, GABRB1, GDF10, GSC, HOXA5, HSD11B2,
HSD17B2, HSPA6, HSPB3, IGF2, INA, KCNMB1, KRT14, KRT34, TMEM119, IGFL3, LOC92196, MFAP5, MEOX1,
MEOX2, MYBPH, MYH3, MYH11, MYL4, NLGN4X, NPAS1, NPPB, OGN, OLR1, PAX2, PAX9, PDE1A, PENK,
PITX2, POSTN, PRELP, PRG4, PROM1, RASD1, RELN, RGMA, RGS1, RPS4Y2, SFRP2, SLITRK6, SMOC1,
SMOC2, SNAP25, SRCRB4D, STMN2, SYT12, TAC1, RSPO3, TNNT2, TRH, TUBB4, UGT2B7, ZD52F10 and ZIC1.
The cell line E45 is positive for the markers: AQP1, CD24, CDH6, COL21A1, CRIP1, DKK2, HTRA3, KRT17, KRT19,
MGP, TAGLN3, PRRX1, S100A4, SOX11, UGT2B7, ZIC1 and ZIC2 and are negative for the markers: AGC1,
ALDH1A1, ANXA8, APCDD1, AREG, ATP8B4, BEX1, BMP4, C3, C6, C7, C20orf103, CDH3, CNTNAP2,
COL15A1, COMP, COP1, CRLF1, METTL7A, DLK1, DPT, EMID1, TMEM100, FMO1, FMO3, FOXF1, FOXF2,
GABRB1, GAP43, GJB2, GSC, HOXA5, HSD11B2, HSPA6, HSPB3, ID4, IFI27, KRT14, LAMC2, IGFL3, MFAP5,
MASP1, MEOX1, MEOX2, MMP1, MYBPH, MYH3, MYH11, NPAS1, NPPB, OGN, OLR1, OSR2, PAX9, PDE1A,
PENK, PITX2, PRG4, PROM1, PTPRN, RARRES1, RASD1, RELN, RGS1, SERPINA3, SFRP2, SLITRK6, SMOC1,
SMOC2, SNAP25, STMN2, TAC1, RSPO3, TRH, TSLP, TUBB4, WISP2 and ZD52F10.
The cell line E50 is positive for the markers: ACTC, BEX1, CD24, CDH6, COL21A1, CRIP1, CRYAB, DKK2, FST,
KRT17, KRT19, LOC92196, POSTN, PTN, S100A4, SFRP2, SRCRB4D, ZIC1 and ZIC2 and are negative for the
markers: AGC1, AKR1C1, ALDH1A1, APCDD1, AQP1, AREG, ATP8B4, CFB, BMP4, C6, C7, CDH3, CLDN11,
CNTNAP2, COMP, COP1, CRLF1, METTL7A, DLK1, DPT, EMID1, TMEM100, FMO3, FOXF1, FOXF2, GABRB1,
GSC, HOXA5, HSD11B2, HSD17B2, HSPA6, HSPB3, IFI27, IFIT3, KRT14, KRT34, LAMC2, TMEM119, IGFL3,
MFAP5, MASP1, MEOX1, MEOX2, MMP1, MYH3, NLGN4X, NPAS1, NPPB, OGN, OLR1, PAX2, PAX9, PENK,
PRG4, PROM1, PTGS2, PTPRN, RARRES1, RASD1, RELN, RGS1, SERPINA3, SLITRK6, SMOC1, SMOC2,
STMN2, SYT12, TAC1, TFPI2, RSPO3, TRH, TSLP, TUBB4, UGT2B7, WISP2 and ZD52F10.
The cell line E51 is positive for the markers: PRSS35, CCDC3, CDH6, CRIP1, CRYAB, DIO2, DKK2, HTRA3, ID4,
KCNMB1, KRT17, KRT19, KRT34, MGP, MYH11, POSTN, PRRX1, S100A4, SOX11 and ZIC2 and are negative for
the markers: AGC1, AKR1C1, ALDH1A1, APCDD1, AREG, ATP8B4, BMP4, C3, C6, C7, C20orf103, CDH3,
CNTNAP2, COP1, CRLF1, CXADR, METTL7A, DLK1, DPT, EMID1, FMO1, FMO3, FOXF1, FOXF2, GABRB1,
GSC, HOXA5, HSD17B2, HSPA6, HSPB3, IFI27, IFIT3, IGF2, IGFBP5, TMEM119, IGFL3, LOC92196, MASP1,
MEOX1, MEOX2, MX1, MYBPH, MYH3, MYL4, NLGN4X, TAGLN3, NPAS1, NPPB, OGN, OLR1, PAX2, PAX9,
PDE1A, PENK, PRG4, PROM1, PTPRN, RARRES1, RASD1, RELN, RGS1, SFRP2, SMOC1, SMOC2, SNAP25,
STMN2, SYT12, TAC1, TFPI2, TNFSF7, TNNT2, TRH, TUBB4, UGT2B7, WISP2 and ZD52F10.
The group of cell lines E68 and E68Bio2 are positive for the markers: CD24, CRIP1, CRYAB, HTRA3, KRT17, KRT19,
TAGLN3, UGT2B7, ZIC1 and ZIC2 and are negative for the markers: AGC1, AREG, ATP8B4, C6, C7, CDH3, COP1,
CRLF1, DLK1, DPT, TMEM100, FMO1, FMO3, FOXF1, FOXF2, GSC, HOXA5, HSD11B2, HSPA6, HSPB3, IGF2,
LAMC2, IGFL3, MEOX1, MEOX2, MMP1, MYBPH, MYH3, NPAS1, OGN, PAX9, PITX2, PRG4, PROM1,
RARRES1, RGS1, SMOC2, TAC1, RSPO3, TRH, TSLP and WISP2.
The group of cell lines C4ELS5.6 and C4ELS5.6Bio2 are positive for the markers: BMP4, COP1, METTL7A,
TMEM100, FOXF1, HSD17B2, HTRA3, IGF2, IGFBP5, IL1R1, KRT19, MASP1, OLR1, PITX2, PODN and TSLP and
are negative for the markers: ACTC, AGC1, ALDH1A1, AQP1, CFB, C6, C7, C20orf103, CDH3, CDH6, CLDN11,
CNTNAP2, COL21A1, COMP, CRLF1, DKK2, DPT, EGR2, EMID1, FMO3, FOXF2, GABRB1, GAP43, GDF10,
GSC, HOXA5, HSPA6, HSPB3, ID4, IFI27, INA, KRT17, KRT34, LAMC2, TMEM119, IGFL3, LOC92196, MFAP5,
MEOX1, MEOX2, MGP, MSX1, MYH3, MYH11, MYL4, IL32, NLGN4X, TAGLN3, NPAS1, NPPB, OGN, PAX2,
PAX9, PDE1A, PENK, PRG4, PROM1, PRRX1, PRRX2, PTPRN, RARRES1, RASD1, RELN, RGMA, RGS1, SFRP2,
SMOC1, SMOC2, SNAP25, SOD3, SYT12, TAC1, RSPO3, THY1, TNFSF7, TNNT2, TRH, TUBB4, UGT2B7, WISP2,
ZD52F10, ZIC1 and ZIC2.
The cell line C4ELS5.8 is positive for the markers: AKR1C1, ALDH1A1, BMP4, C3, COP1, METTL7A, TMEM100,
FOXF1, HSD17B2, HTRA3, ICAM5, IFIT3, IGF2, IGFBP5, IL1R1, KRT19, MASP1, MX1, OLR1, PODN, STMN2,
TFPI2 and THY1 and are negative for the markers: ACTC, AGC1, APCDD1, BEX1, C6, C7, PRSS35, C20orf103,
CCDC3, CD24, CDH3, CLDN11, CNTNAP2, COL21A1, COMP, CRIP1, CRLF1, DKK2, DLK1, DPT, EMID1,
FGFR3, FMO3, FOXF2, GABRB1, GAP43, GDF10, GSC, HOXA5, HSD11B2, HSPA6, HSPB3, ID4, INA, KCNMB1,
KRT14, KRT17, TMEM119, IGFL3, LOC92196, MFAP5, MEOX1, MEOX2, MGP, MSX2, MYH3, MYH11, MYL4,
IL32, NLGN4X, TAGLN3, NPPB, OGN, PAX2, PAX9, PDE1A, PENK, POSTN, PRRX1, PRRX2, PTPRN, RARRES1,
RASD1, RELN, RGMA, RGS1, SLITRK6, SMOC1, SMOC2, SOD3, SOX11, SYT12, TAC1, RSPO3, TNFSF7,
TNNT2, TRH, TUBB4, UGT2B7, WISP2, ZD52F10, ZIC1 and ZIC2.
The cell line C4ELSR13 is positive for the markers: AKR1C1, ANXA8, AREG, BMP4, C3, COP1, METTL7A, FMO3,
FOXF1, HTRA3, IFI27, IFIT3, IGF2, IL1R1, KRT19, MASP1, MX1, MYBPH, OLR1, PITX2, PODN, S100A4 and
TFPI2 and are negative for the markers: AGC1, APCDD1, AQP1, ATP8B4, C6, C20orf103, CD24, CDH3, CDH6,
CLDN11, CNTNAP2, COL15A1, COL21A1, COMP, CRIP1, CRLF1, CRYAB, DKK2, DLK1, DPT, EGR2, EMID1,
FGFR3, TMEM100, FMO1, FOXF2, GABRB1, GAP43, GDF10, GSC, HOXA5, HSD11B2, HSD17B2, HSPA6,
HSPB3, ID4, INA, KIAA0644, KRT14, KRT17, IGFL3, LOC92196, MFAP5, MEOX1, MEOX2, MGP, MSX1, MSX2,
MYH3, MYH11, MYL4, IL32, NLGN4X, TAGLN3, NPAS1, NPPB, OGN, OSR2, PAX2, PAX9, PDE1A, PENK,
POSTN, PROM1, PRRX1, PTPRN, RARRES1, RASD1, RELN, RGMA, RGS1, RPS4Y2, SERPINA3, SLITRK6,
SMOC2, SNAP25, SOD3, SOX11, STMN2, SYT12, TAC1, RSPO3, THY1, TNNT2, TRH, TUBB4, UGT2B7,
ZD52F10, ZIC1 and ZIC2.
The cell line C4ELSR18 is positive for the markers: AQP1, BEX1, BMP4, C20orf103, CDH6, FST, HOXA5, IGF2,
IGFBP5, OLR1, OSR2, PDE1A, PRRX2, S100A4, SFRP2, SLITRK6, TFPI2 and ZIC2 and are negative for the markers:
AGC1, ALDH1A1, ANXA8, APCDD1, ATP8B4, CFB, C6, CCDC3, CD24, CDH3, CLDN11, CNTNAP2, COL15A1,
COMB, COP1, CRLF1, CRYAB, DLK1, DPT, EGR2, EMID1, TMEM100, FOXF1, GABRB1, GAP43, GDF10, GSC,
HSD11B2, HSD17B2, HSPA6, HSPB3, ID4, IFI27, IFIT3, KCNMB1, KRT14, KRT17, KRT34, TMEM119, IGFL3,
LOC92196, MFAP5, MASP1, MEOX1, MEOX2, MSX1, MSX2, MX1, MYH3, MYH11, MYL4, IL32, NPAS1, NPPB,
OGN, PAX2, PAX9, PENK, PITX2, PODN, PRG4, PTPRN, RARRES1, RASD1, RELN, RGS1, SERPINA3, SMOC1,
SMOC2, SOD3, SOX11, STMN2, SYT12, TAC1, THY1, TNFSF7, TNNT2, TRH, TUBB4, UGT2B7, ZD52F10 and
ZIC1.
The group of cell lines EN11 and W10 are positive for the markers: DLK1, FOXF1, FST, GABRB1, GDF5, HTRA3,
IGF2, IGFBP5, IL1R1, POSTN, PTN, SOX11, SRCRB4D and TFPI2 and are negative for the markers: ACTC, AGC1,
ALDH1A1, ANXA8, APCDD1, AQP1, AREG, CFB, BMP4, C3, C6, C7, CCDC3, CD24, CDH6, CLDN11, CNTNAP2,
COL15A1, COMP, COP1, CRYAB, DKK2, DPT, EGR2, EMID1, FGFR3, FMO1, FMO3, FOXF2, GAP43, GDF10,
GSC, HSD11B2, HSD17B2, HSPA6, HSPB3, ID4, IFI27, INA, KCNMB1, KRT14, KRT17, KRT34, IGFL3,
LOC92196, MEOX1, MEOX2, MX1, MYBPH, MYH3, MYH11, MYL4, IL32, NLGN4X, NPAS1, NPPB, OLR1,
PAX2, PAX9, PENK, PITX2, PRELP, PROM1, RARRES1, RASD1, RELN, RGS1, SMOC1, SMOC2, STMN2, SYT12,
TAC1, THY1, TNFSF7, TNNT2, TRH, TUBB4, UGT2B7, WISP2, ZIC1 and ZIC2.
The group of cell lines EN7, EN13Bio1b, EN13Bio2c and EN13Bio3c are positive for the markers: CDH6, DLK1,
FOXF1, FST, HTRA3, IGF2, IL1R1, MSX1, POSTN, SOD3, ZIC1 and ZIC2 and are negative for the markers: ACTC,
ALDH1A1, ANXA8, ATP8B4, BMP4, C3, C20orf103, CCDC3, CD24, CDH3, CLDN11, CNTNAP2, COMP, CRYAB,
DIO2, DKK2, GSC, HOXA5, HSD11B2, HSD17B2, HSPA6, HSPB3, IFI27, INA, KRT14, KRT17, KRT34, IGFL3,
LOC92196, MFAP5, MEOX1, MEOX2, MGP, MMP1, MX1, MYH3, MYH11, MYL4, IL32, NPAS1, NPPB, OLR1,
PAX2, PAX9, PDE1A, PENK, PITX2, PROM1, RELN, SFRP2, SMOC2, STMN2, TAC1, RSPO3, THY1, TNFSF7,
TNNT2, TRH, TUBB4 and ZD52F10.
The cell line EN16 is positive for the markers: COL15A1, DIO2, DPT, FMO3, FOXF1, FOXF2, FST, HSPB3, HTRA3,
IGF2, IL1R1, TMEM119, MGP, MMP1, PODN and PRRX2 and are negative for the markers: ACTC, AGC1, AKR1C1,
ALDH1A1, ANXA8, AQP1,, AREG, ATP8B4, BEX1, CFB, C3, C6, C7, C20orf103, CCDC3, CD24, CDH3, CLDN11,
CNTNAP2, COMP, CRIP1, CRLF1, DKK2, EMID1, FGFR3, TMEM100, GABRB1, GAP43, GDF5, GDF10, GJB2,
GSC, HOXA5, HSD11B2, HSD17B2, HSPA6, ID4, IFI27, KCNMB1, KRT14, KRT17, KRT34, LAMC2, IGFL3,
LOC92196, MFAP5, MEOX1, MEOX2, MYBPH, MYH3, MYH11, MYL4, IL32, NLGN4X, TAGLN3, NPAS1, NPPB,
PAX2, PAX9, PENK, PITX2, POSTN, PTGS2, PTPRN, RARRES1, RASD1, RGS1, SMOC1, SMOC2, SNAP25,
STMN2, TAC1, RSPO3, THY1, TNFSF7, TNNT2, TRH, TUBB4, UGT2B7, ZD52F10, ZIC1 and ZIC2.
The group of cell lines EN1, EN1Bio2 and EN18 are positive for the markers: DIO2, DLK1, FOXF1, GDF5, HTRA3,
IGF2, IL1R1, MGP, POSTN, PRRX2 and SRCRB4D and are negative for the markers: ACTC, AGC1, ALDH1A1,
ANXA8, AQP1, CFB, C20orf103, CCDC3, CD24, CLDN11, CNTNAP2, CRYAB, CXADR, DKK2, GABRB1, GAP43,
GDF10, GSC, HSD11B2, HSD17B2, HSPA6, IFI27, INA, KCNMB1, KRT14, KRT17, KRT34, IGFL3, LOC92196,
MFAP5, MEOX1, MEOX2, MX1, MYH3, MYH11, MYL4, NPAS1, NPPB, PAX2, PAX9, PENK, PITX2, PROM1,
RASD1, RGS1, SMOC1, SMOC2, STMN2, TAC1, RSPO3, THY1, TNFSF7, TNNT2, TRH, TUBB4, UGT2B7,
ZD52F10, ZIC1 and ZIC2.
The cell line EN19 is positive for the markers: CDH6, COL15A1, COL21A1, DLK1, FOXF1, FST, GDF5, IGF2,
TMEM119, MSX1, RGMA, SERPINA3, SOD3, ZIC1 and ZIC2 and are negative for the markers: ACTC, AGC1,
ANXA8, AQP1, ATP8B4, C3, C6, C7, C20orf103, CD24, CDH3, CLDN11, CNTNAP2, CRIP1, CXADR, DIO2, DKK2,
EMID1, TMEM100, GABRB1, GAP43, GJB2, GSC, HOXA5, HSD11B2, HSD17B2, HSPA6, HSPB3, IFI27, INA,
KCNMB1, KRT14, KRT17, KRT19, KRT34, IGFL3, LOC92196, MFAP5, MEOX1, MEOX2, MGP, MX1, MYH3,
MYH11, MYL4, IL32, NLGN4X, NPPB, OLR1, OSR2, PAX2, PAX9, PDE1A, PENK, PROM1, RARRES1, RASD1,
RELN, RGS1, SLITRK6, SMOC1, SMOC2, SNAP25, STMN2, SYT12, TAC1, RSPO3, THY1, TNFSF7, TNNT2, TRH,
TUBB4, UGT2B7 and ZD52F10.
The cell line EN2 is positive for the markers: FST, GDF5, HTRA3, IGF2, IGFBP5, IL1R1, PRRX2, PTN, SFRP2,
SOX11, SRCRB4D, TFPI2 and RSPO3 and are negative for the markers: ACTC, AGC1, AKR1C1, ALDH1A1, ANXA8,
APCDD1, AREG, ATP8B4, CFB, C3, C6, C7, PRSS35, C20orf103, CCDC3, CD24, CDH6, CLDN11, COMP, COP1,
CRLF1, CXADR, DKK2, DPT, EGR2, EMID1, TMEM100, FMO1, FOXF2, GAP43, GDF10, GJB2, GSC, HOXA5,
HSD11B2, HSD17B2, HSPA6, HSPB3, ICAM5, IFI27, INA, KRT14, KRT17, KRT19, KRT34, TMEM119, IGFL3,
LOC92196, MFAP5, MEOX1, MEOX2, MX1, MYBPH, MYH3, MYH11, MYL4, NLGN4X, TAGLN3, NPAS1, NPPB,
OGN, OLR1, PAX2, PAX9, PDE1A, PENK, PITX2, POSTN, PRELP, PRG4, PTGS2, RARRES1, RASD1, RELN,
RGS1, SMOC1, SMOC2, SNAP25, STMN2, SYT12, TAC1, THY1, TNFSF7, TNNT2, TRH, TSLP, TUBB4, UGT2B7,
ZD52F10, ZIC1 and ZIC2.
The cell line EN25 is positive for the markers: CDH6, CNTNAP2, COL15A1, COL21A1, DLK1, FOXF1, FST, HTRA3,
IGF2, SERPINA3, SRCRB4D, TFPI2, ZIC1 and ZIC2 and are negative for the markers: ACTC, AGC1, AKR1C1,
ALDH1A1, AQP1, ATP8B4, C3, C6, C7, C20orf103, CCDC3, CD24, CDH3, CLDN11, CRIP1, DIO2, DKK2, EMID1,
FOXF2, GSC, HOXA5, HSD11B2, HSD17B2, HSPA6, HSPB3, IFI27, IFIT3, INA, KCNMB1, KRT14, KRT17,
KRT34, IGFL3, LOC92196, MFAP5, MEOX1, MEOX2, MGP, MMP1, MX1, MYBPH, MYH3, MYH11, MYL4, IL32,
NLGN4X, NPPB, OLR1, PAX2, PAX9, PENK, PITX2, PRELP, PROM1, PRRX1, PTN, RARRES1, RASD1, RELN,
SFRP2, SLITRK6, SMOC2, STMN2, TAC1, RSPO3, THY1, TNFSF7, TNNT2, TRH, TUBB4, UGT2B7 and ZD52F10.
The cell line EN26 is positive for the markers: DIO2, DPT, FMO3, FOXF1, FOXF2, FST, GDF5, HTRA3, IGF2, IL1R1,
TMEM119, PODN, PRRX1, PRRX2, SFRP2, SOD3 and SRCRB4D and are negative for the markers: ACTC, AGC1,
AKR1C1, ALDH1A1, ANXA8, AQP1, ATP8B4, BEX1, C3, C6, C7, C20orf103, CCDC3, CD24, CLDN11, CNTNAP2,
COL21A1, COMP, CRIP1, CXADR, DKK2, GABRB1, GAP43, GDF10, GJB2, GSC, HOXA5, HSD11B2, HSD17B2,
HSPA6, ID4, IFI27, INA, KCNMB1, KRT14, KRT17, KRT19, KRT34, LAMC2, IGFL3, LOC92196, MFAP5, MEOX1,
MEOX2, MMP1, MX1, MYBPH, MYH3, MYH11, MYL4, NLGN4X, NPAS1, NPPB, PAX2, PAX9, PENK, PITX2,
PROM1, PTGS2, PTPRN, RARRES1, RASD1, RELN, RGS1, SLITRK6, SMOC1, SMOC2, STMN2, TAC1, RSPO3,
THY1, TNFSF7, TNNT2, TRH, TUBB4, UGT2B7, ZD52F10, ZIC1 and ZIC2.
The cell line EN27 is positive for the markers: DIO2, FMO3, FOXF1, FOXF2, FST, HSPB3, HTRA3, IGF2, IL1R1,
TMEM119, MSX2, OGN, PODN, PRELP, PRRX2, SERPINA3 and SLITRK6 and are negative for the markers:, ACTC,
AGC1, ALDH1A1, ANXA8, AQP1, AREG, ATP8B4, CFB, C3, C6, C7, C20orf103, CCDC3, CD24, CDH3, CDH6,
CLDN11, CNTNAP2, CRIP1, CRLF1, DKK2, EMID1, FGFR3, TMEM100, GABRB1, GAP43, GDF10, GJB2, GSC,
HOXA5, HSD11B2, HSD17B2, HSPA6, ICAM5, ID4, IFI27, IFIT3, IGFBP5, INA, KCNMB1, KRT14, KRT17,
KRT19, KRT34, LAMC2, IGFL3, LOC92196, MFAP5, MASP1, MEOX1, MEOX2, MMP1, MX1, MYBPH, MYH3,
MYH11, MYL4, IL32, NLGN4X, NPAS1, NPPB, OLR1, PAX2, PAX9, PENK, PITX2, PROM1, RARRES1, RASD1,
RELN, RGS1, SFRP2, SMOC1, SMOC2, STMN2, TAC1, RSPO3, THY1, TNFSF7, TNNT2, TRH, TUBB4, UGT2B7,
ZD52F10, ZIC1 and ZIC2.
The cell line EN28 is positive for the markers: COL15A1, COL21A1, DIO2, FOXF1, FOXF2, FST, HSPB3, HTRA3,
IGF2, IGFBP5, IL1R1, TMEM119, PODN, PRRX1, PTN, SFRP2 and SOX11 and are negative for the markers: ACTC,
AGC1, AKR1C1, ALDH1A1, ANXA8, AQP1, AREG, ATP8B4, CFB, BMP4, C3, C6, C7, C20orf103, CCDC3, CD24,
CDH3, CDH6, CLDN11, CNTNAP2, COP1, CRIP1, DKK2, EMID1, TMEM100, GAP43, GDF10, GJB2, GSC,
HOXA5, HSD11B2, HSD17B2, HSPA6, ID4, IFI27, INA, KCNMB1, KIAA0644, KRT14, KRT17, KRT34, IGFL3,
LOC92196, MFAP5, MEOX1, MEOX2, MGP, MMP1, MX1, MYBPH, MYH3, MYH11, MYL4, IL32, NLGN4X,
NPPB, OLR1, OSR2, PAX2, PAX9, PDE1A, PENK, PITX2, POSTN, PRELP, PRG4, PROM1, PTGS2, RARRES1,
RELN, RGS1, SLITRK6, SMOC1, SMOC2, STMN2, SYT12, TAC1, RSPO3, TNFSF7, TNNT2, TRH, TSLP, TUBB4,
UGT2B7, ZD52F10, ZIC1 and ZIC2.
The cell line EN31 is positive for the markers: CDH6, COL21A1, DLK1, FMO3, FOXF1, FST, GDF5, HTRA3, IGF2,
IL1R1, MSX1, MSX2, OGN, OSR2, PRRX2, SERPINA3, SLITRK6, SOD3, TSLP, ZIC1 and ZIC2 and are negative for
the markers: ACTC, AGC1, ALDH1A1, ANXA8, AQP1, ATP8B4, BEX1, BMP4, C3, C6, C7, PRSS35, C20orf103,
CCDC3, CD24, CDH3, CLDN11, CNTNAP2, COMP, CRIP1, CRLF1, CRYAB, CXADR, DIO2, DKK2, EMID1,
TMEM100, GAP43, GDF10, GJB2, GSC, HOXA5, HSD11B2, HSD17B2, HSPA6, HSPB3, ICAM5, ID4, IFI27, INA,
KRT14, KRT17, KRT19, KRT34, LAMC2, IGFL3, LOC92196, MFAP5, MEOX1, MEOX2, MGP, MMP1, MX1,
MYBPH, MYH3, MYH11, MYL4, IL32, NLGN4X, TAGLN3, NPAS1, NPPB, OLR1, PAX2, PAX9, PENK, PITX2,
PROM1, PTGS2, RARRES1, RASD1, RELN, SFRP2, SMOC2, SNAP25, STMN2, SYT12, TAC1, RSPO3, TNFSF7,
TNNT2, TRH, TUBB4, UGT2B7 and ZD52F10.
The cell line EN38 is positive for the markers: BEX1, CDH6, COL21A1, DLK1, FOXF1, FST, GDF5, HTRA3, IGF2,
IL1R1, TMEM119, MGP, MSX1, OGN, PODN, POSTN, PRRX1, PRRX2, RGMA, SERPINA3, SOD3 and TSLP and
are negative for the markers: ACTC, AGC1, AKR1C1, ALDH1A1, ANXA8, AQP1, AREG, ATP8B4, BMP4, C3, C6,
C7, C20orf103, CCDC3, CD24, CDH3, CLDN11, CNTNAP2, CRIP1, DIO2, DKK2, DPT, GABRB1, GAP43, GDF10,
GJB2, GSC, HOXA5, HSD11B2, HSD17B2, HSPA6, HSPB3, ID4, IFI27, INA, KCNMB1, KRT14, KRT17, KRT34,
IGFL3, LOC92196, MFAP5, MEOX1, MEOX2, MX1, MYBPH, MYH3, MYH11, MYL4, IL32, NLGN4X, NPPB,
OLR1, PAX2, PAX9, PDE1A, PENK, PITX2, PRELP, PRG4, PROM1, RASD1, RELN, RGS1, SFRP2, SLITRK6,
SMOC1, SMOC2, SNAP25, STMN2, SYT12, TAC1, RSPO3, THY1, TNFSF7, TNNT2, TRH, TUBB4, ZD52F10, ZIC1
and ZIC2.
The cell line EN4 is positive for the markers: COL21A1, DLK1, FMO1, FMO3, FOXF1, FOXF2, FST, GDF5, HTRA3,
IGF2, IGFBP5, IL1R1, TMEM119, MGP, MSX1, OGN, PODN, PRRX1, PRRX2, PTN, RGMA, SOD3 and TSLP and
are negative for the markers: ACTC, AGC1, AKR1C1, ALDH1A1, ANXA8, AQP1, AREG, CFB, BMP4, C3, C6, C7,
C20orf103, CCDC3, CD24, CDH3, CLDN11, CNTNAP2, CRIP1, DIO2, DKK2, DPT, EMID1, FGFR3, TMEM100,
GABRB1, GAP43, GDF10, GJB2, GSC, HOXA5, HSD11B2, HSD17B2, HSPA6, HSPB3, ID4, IFI27, INA, KCNMB1,
KRT14, KRT17, KRT34, LAMC2, IGFL3, LOC92196, MFAP5, MASP1, MEOX1, MEOX2, MX1, MYBPH, MYH3,
MYH11, MYL4, IL32, NLGN4X, NPAS1, NPPB, OLR1, PAX2, PAX9, PENK, PROM1, PTGS2, RARRES1, RASD1,
RGS1, SFRP2, SMOC1, SMOC2, SNAP25, STMN2, TAC1, RSPO3, THY1, TNFSF7, TNNT2, TRH, TUBB4, UGT2B7
and ZD52F10.
The cell line EN42 is positive for the markers: COL15A1, COL21A1, FMO3, FOXF1, FST, GDF5, HTRA3, IGF2,
IL1R1, TMEM119, MGP, OGN, PODN, PRRX1, PRRX2, PTN, RGMA, SERPINA3, SNAP25 and SOD3 and are
negative for the markers: ACTC, AGC1, AKR1C1, ALDH1A1, ANXA8, AQP1, ATP8B4, BMP4, C3, C6, C7,
C20orf103, CCDC3, CD24, CDH3, CLDN11, CNTNAP2, COMP, CXADR, DIO2, DKK2, DPT, EMID1, FGFR3,
TMEM100, GAP43, GDF10, GSC, HOXA5, HSD11B2, HSD17B2, HSPA6, HSPB3, ID4, IFI27, INA, KCNMB1,
KRT14, KRT17, KRT19, KRT34, LAMC2, IGFL3, LOC92196, MFAP5, MASP1, MEOX1, MEOX2, MMP1, MX1,
MYBPH, MYH3, MYH11, MYL4, IL32, NLGN4X, NPAS1, NPPB, OLR1, PAX9, PENK, PITX2, PRG4, PROM1,
RARRES1, RASD1, RELN, RGS1, SMOC1, SMOC2, STMN2, RSPO3, THY1, TNFSF7, TNNT2, TRH, TUBB4,
UGT2B7, ZD52F10, ZIC1 and ZIC2.
The cell line EN47 is positive for the markers; CDH6, COP1, DLK1, FMO3, FOXF1, FST, HTRA3, IGF2, IL1R1,
MSX1, POSTN, PTPRN, RGS1, SOD3, TFPI2, TSLP, ZIC1 and ZICZ and are negative for the markers: AGC1,
ALDH1A1, APCDD1, BMP4, C3, C20orf103, CCDC3, CD24, CDH3, DIO2, DKK2, FOXF2, GSC, HOXA5,
HSD11B2, HSD17B2, HSPA6, HSPB3, IFI27, INA, KCNMB1, KRT14, KRT17, KRT34, LAMC2, TMEM119, IGFL3,
LOC92196, MFAP5, MEOX1, MEOX2, MX1, MYH3, MYH11, MYL4, IL32, NLGN4X, NPAS1, NPPB, OLR1, PAX2,
PAX9, PENK, PITX2, PRELP, PROM1, RARRES1, SFRP2, SMOC2, STMN2, TAC1, RSPO3, THY1, TNFSF7,
TNNT2, TRH, TUBB4, UGT2B7 and ZD52F10.
The cell line EN5 is positive for the markers: COL21A1, DLK1, FMO3, FOXF1, FOXF2, FST, HTRA3, IGF2, IL1R1,
KIAA0644, TMEM119, MGP, MSX1, MSX2, OGN, PRRX1 and PRRX2 and are negative for the markers: ACTC,
AGC1, AKR1C1, ALDH1A1, ANXA8, AQP1, AREG, BMP4, C3, C6, C7, C20orf103, CCDC3, CD24, CDH3,
CLDN11, CNTNAP2, COMP, CRIP1, CRLF1, CRYAB, CXADR, DKK2, GABRB1, GAP43, GDF10, GJB2, GSC,
HOXA5, HSD11B2, HSD17B2, HSPA6, HSPB3, ID4, IFI27, INA, KCNMB1, KRT14, KRT17, KRT34, LAMC2,
IGFL3, LOC92196, MFAP5, MEOX1, MEOX2, MMP1, MX1, MYH3, MYH11, MYL4, IL32, NLGN4X, NPAS1,
NPPB, PAX2, PAX9, PENK, PITX2, PRELP, PRG4, PROM1, RASD1, RELN, RGS1, SMOC1, SMOC2, STMN2,
SYT12, TAC1, TFPI2, RSPO3, THY1, TNFSF7, TNNT2, TRH, TUBB4, UGT2B7, ZD52F10 and ZIC1.
The cell line EN50 is positive for the markers: BEX1, CDH6, COL21A1, DIO2, FMO1, FOXF1, FOXF2, FST, GDF5,
HTRA3, IGF2, IGFBP5, IL1R1, KRT19, TMEM119, MASP1, MGP, MSX1, PODN, PRRX2, PTPRN, SERPINA3,
SOD3, WISP2, ZIC1 and ZIC2 and are negative for the markers: ACTC, AGC1, ALDH1A1, APCDD1, AQP1, BMP4,
C3, C6, C20orf103, CDH3, CLDN11, CNTNAP2, COMP, DKK2, DPT, EGR2, EMID1, TMEM100, GABRB1, GAP43,
GDF10, GSC, HOXA5, HSD11B2, HSPA6, HSPB3, IFI27, KIAA0644, KRT17, KRT34, IGFL3, LOC92196, MFAP5,
MEOX1, MEOX2, MX1, MYBPH, MYH3, MYH11, NLGN4X, NPPB, OGN, OSR2, PAX2, PAX9, PDE1A, PENK,
PITX2, PRELP, PROM1, PRRX1, RARRES1, RASD1, RGS1, SMOC2, SNAP25, STMN2, SYT12, TAC1, RSPO3,
TNFSF7, TNNT2, TRH, TUBB4, UGT2B7 and ZD52F10.
The cell line EN51 is positive for the markers: CDH6, DLK1, FMO1, FMO3, FOXF1, FST, HTRA3, IGF2, IL1R1,
MSX1, MSX2, OGN, SERPINA3, SOD3, TSLP, ZIC1 and ZIC2 and are negative for the markers: ACTC, AGC1,
AKR1C1, ALDH1A1, ANXA8, APCDD1, AQP1, ATP8B4, CFB, C3, C6, C20orf103, CCDC3, CD24, CDH3, CLDN11,
CRIP1, CRYAB, CXADR, DIO2, DKK2, DPT, EMID1, TMEM100, FOXF2, GABRB1, GSC, HOXA5, HSD11B2,
HSD17B2, HSPA6, HSPB3, ID4, IFI27, INA, KCNMB1, KRT14, KRT17, KRT19, KRT34, LAMC2, IGFL3,
LOC92196, MFAP5, MEOX1, MEOX2, MGP, MMP1, MX1, MYH3, MYH11, MYL4, IL32, NLGN4X, NPAS1, NPPB,
OLR1, PAX2, PAX9, PDE1A, PENK, PITX2, PRELP, PROM1, PTGS2, RARRES1, RASD1, RELN, RGS1, SFRP2,
SMOC2, STMN2, TAC1, RSPO3, THY1, TNFSF7, TNNT2, TRH, TUBB4, UGT2B7 and ZD52F10.
The cell line EN53 is positive for the markers: BEX1, COL21A1, FST, GDF5, HTRA3, ICAM5, KRT19, TMEM119,
PTPRN, SERPINA3, SOD3 and ZIC2 and are negative for the markers: ACTC, AGC1, ALDH1A1, APCDD1, AQP1,
ATP8B4, BMP4, C3, C6, C7, C20orf103, CCDC3, CDH3, CLDN11, CNTNAP2, COP1, CRYAB, DIO2, DKK2, DPT,
EMID1, FGFR3, TMEM100, FMO3, FOXF2, GABRB1, GAP43, GJB2, GSC, HOXA5, HSPA6, HSPB3, ID4, IFI27,
INA, KCNMB1, KIAA0644, KRT14, KRT17, KRT34, IGFL3, LOC92196, MFAP5, MEOX1, MEOX2, MGP, MMP1,
MX1, MYBPH, MYH3, MYH11, MYL4, IL32, NLGN4X, NPPB, OGN, OLR1, OSR2, PAX2, PAX9, PDE1A, PENK,
PITX2, POSTN, PRELP, PROM1, PTN, RASD1, RELN, RGS1, SLITRK6, SMOC2, STMN2, SYT12, TAC1, RSPO3,
THY1, TNFSF7, TNNT2, TRH, TUBB4, UGT2B7, ZD52F10 and ZIC1.
The cell line EN55 is positive for the markers: DIO2, FOXF1, FOXF2, FST, GDF5, HTRA3, IGF2, IL1R1, KIAA0644,
MGP, MSX2, PODN, PRRX2, PTN, SLITRK6 and SRCRB4D and are negative for the markers: ACTC, AGC1,
AKR1C1, ALDH1A1, ANXA8, AQP1, ATP8B4, CFB, BMP4, C6, C7, C20orf103, CCDC3, CD24, CDH3, CLDN11,
CNTNAP2, CRIP1, CRYAB, DKK2, FGFR3, FMO1, GABRB1, GAP43, GDF10, GSC, HOXA5, HSD11B2, HSD17B2,
HSPA6, HSPB3, ICAM5, ID4, IFI27, INA, KCNMB1, KRT14, KRT17, KRT34, LAMC2, IGFL3, LOC92196, MFAP5,
MEOX1, MEOX2, MX1, MYBPH, MYH3, MYH11, MYL4, IL32, NLGN4X, NPAS1, NPPB, OLR1, PAX2, PAX9,
PENK, PITX2, POSTN, PROM1, PRRX1, PTGS2, RARRES1, RASD1, RELN, RGS1, SFRP2, SMOC1, SMOC2,
SOD3, STMN2, SYT12, TAC1, RSPO3, THY1, TNFSF7, TNNT2, TRH, TUBB4, UGT2B7, ZD52F10, ZIC1 and ZIC2.
The group of cell lines H9.Bio1 and H9.Bio2 are positive for the markers: ACTC, BEX1, CD24, CDH3, CNTNAP2,
CXADR, METTL7A, FGFR3, FST, GAP43, INA, KRT19, NLGN4X, PROM1, PTN, PTPRN, RGMA, SFRP2, SOX11,
SRCRB4D, ZD52F10 and ZIC2 and are negative for the markers: AGC1, ALDH1A1, ANXA8, APCDD1, AQP1,
AREG, ATP8B4, CFB, C6, C7, PRSS35, C20orf103, CDH6, CLDN11, COL15A1, COL21A1, COP1, DIO2, DKK2,
DPT, EGR2, TMEM100, FMO1, FMO3, FOXF1, FOXF2, GABRB1, GDF10, GJB2, HSD17B2, HSPA6, HSPB3, IFI27,
IFIT3, IGF2, IL1R1, KRT14, KRT17, KRT34, TMEM119, IGFL3, LOC92196, MEOX1, MEOX2, MGP, MMP1,
MSX1, MSX2, MX1, MYBPH, MYH3, MYH11, OGN, OLR1, OSR2, PAX2, PAX9, PDE1A, PENK, POSTN, PRELP,
PRG4, PRRX1, PTGS2, RARRES1, RELN, RGS1, SERPINA3, SLITRK6, SMOC1, SNAP25, RSPO3, TNFSF7,
TNNT2, TRH, TUBB4, UGT2B7 and WISP2.
The cell line J13 is positive for the markers: CDH6, CLDN11, FST, GDF5, IGF2, MMP1, PRRX1, PRRX2, RGMA,
SLITRK6, TFPI2 and ZIC2 and are negative for the markers: ACTC, AGC1, ALDH1A1, ANXA8, AQP1, AREG,
ATP8B4, CFB, C3, C6, PRSS35, C20orf103, CCDC3, CD24, CDH3, CNTNAP2, COL15A1, COMP, COP1, CRLF1,
CRYAB, DIO2, METTL7A, DKK2, DLK1, DPT, EGR2, EMID1, FGFR3, TMEM100, FMO1, FOXF1, GABRB1,
GAP43, GDF10, GSC, HOXA5, HSD11B2, HSD17B2, HSPA6, HSPB3, ID4, IFI27, IGFBP5, KCNMB1, KIAA0644,
KRT14, KRT17, KRT34, LAMC2, IGFL3, LOC92196, MFAP5, MEOX1, MEOX2, MGP, MYBPH, MYH3, MYH11,
MYL4, IL32, NPAS1, NPPB, OGN, OLR1, PAX2, PAX9, PENK, PITX2, POSTN, PRELP, PRG4, PROM1, PTGS2,
PTPRN, RARRES1, RASD1, RELN, RGS1, RPS4Y2, SFRP2, SMOC1, SMOC2, SRCRB4D, STMN2, TAC1, RSPO3,
THY1, TNFSF7, TNNT2, TRH, TUBB4, UGT2B7, ZD52F10 and ZIC1.
The cell line J16Bio2 is positive for the markers: BEX1, BMP4, CCDC3, CDH6, CLDN11, COL21A1, CRYAB, FMO3,
FST, ICAM5, IGF2, KRT17, TMEM119, POSTN, SERPINA3, SFRP2, SYT12, TFPI2, UGT2B7 and ZIC2 and are
negative for the markers: AGC1, ALDH1A1, APCDD1, AQP1, AREG, ATP8B4, C3, C6, C20orf103, CD24, CDH3,
CNTNAP2, COMP, CRLF1, METTL7A, DLK1, DPT, EMID1, FGFR3, TMEM100, FMO1, FOXF1, FOXF2, GABRB1,
GAP43, GDF10, GJB2, GSC, HOXA5, HSD11B2, HSPA6, HSPB3, HTRA3, ID4, IFI27, KIAA0644, KRT14, KRT34,
IGFL3, LOC92196, MEOX1, MEOX2, MSX1, MYBPH, MYH3, NLGN4X, NPPB, OGN, PAX2, PAX9, PDE1A,
PENK, PITX2, PRELP, PRG4, PROM1, PTPRN, RARRES1, RASD1, RELN, RGS1, SMOC1, SMOC2, STMN2,
TAC1, THY1, TNFSF7, TRH, TUBB4, WISP2 and ZD52F10.
The cell line J8 is positive for the markers: BEX1, BMP4, CLDN11, CRYAB, IGF2, INA, KRT19, MX1, IL32,
TAGLN3, SFRP2, TSLP and UGT2B7 and is negative for the markers: AGC1, ALDH1A1, ANXA8, APCDD1,
ATP8B4, CFB, C3, C6, C7, C20orf103, CCDC3, CDH3, CNTNAP2, COL15A1, COL21A1, COMP, COP1, CRLF1,
DIO2, METTL7A, DKK2, DLK1, DPT, EGR2, EMID1, FGFR3, TMEM100, FMO1, FMO3, FOXF1, FOXF2,
GABRB1, GAP43, GSC, HOXA5, HSD11B2, HSPA6, HSPB3, ID4, IFI27, IGFBP5, KCNMB1, KIAA0644, KRT14,
KRT34, IGFL3, LOC92196, MFAP5, MASP1, MEOX1, MEOX2, MGP, MMP1, MSX1, MYH3, MYH11, MYL4,
NPAS1, NPPB, OGN, OLR1, OSR2, PAX2, PAX9, PENK, PITX2, PRELP, PROM1, PRRX1, PTGS2, PTN, PTPRN,
RARRES1, RGMA, RGS1, SLITRK6, SMOC1, SMOC2, SNAP25, STMN2, TAC1, TNNT2, TRH, TUBB4, WISP2 and
ZD52F10.
The cell line MW1 is positive for the markers: APCDD1, BEX1, BMP4, C3, CD24, CDH3, CRLF1, CRYAB, DIO2,
METTL7A, TMEM100, FOXF1, FST, GJB2, IGF2, IGFBP5, IL1R1, KIAA0644, KRT19, TMEM119, OLR1, PODN,
PROM1, SERPINA3, SNAP25, SRCRB4D, STMN2, TFPI2 and THY1 and are negative for the markers: ACTC, AGC1,
AKR1C1, ALDH1A1, AQP1, AREG, ATP8B4, C6, C7, PRSS35, C20orf103, CCDC3, CDH6, CLDN11, CNTNAP2,
COL15A1, COL21A1, COMP, COP1, CXADR, DKK2, DLK1, DPT, EGR2, EMID1, FGFR3, FMO1, FMO3, FOXF2,
GABRB1, GAP43, GDF5, GDF10, GSC, HOXA5, HSD11B2, HSD17B2, HSPA6, HSPB3, HTRA3, ICAM5, ID4,
IFI27, INA, KCNMB1, KRT14, KRT17, KRT34, IGFL3, LOC92196, MFAP5, MASP1, MEOX1, MEOX2, MGP,
MMP1, MSX2, MYBPH, MYH3, MYH11, MYL4, IL32, NLGN4X, TAGLN3, NPAS1, NPPB, OGN, OSR2, PAX2,
PAX9, PENK, POSTN, PRELP, PRG4, PRRX1, PRRX2, PTGS2, PTPRN, RARRES1, RELN, RGS1, SFRP2,
SLITRK6, SMOC1, SMOC2, SOD3, SYT12, TAC1, RSPO3, TNFSF7, TNNT2, TRH, TSLP, TUBB4, UGT2B7,
WISP2, ZD52F10, ZIC1 and ZIC2.
The cell line MW2 is positive for the markers: C6, C7, CRLF1, DIO2, METTL7A, FMO1, FMO3, FOXF1, FOXF2,
HTRA3, IGF2, IL1R1, TMEM119, MGP, OGN, PRRX2, RGMA, SFRP2, SYT12 and TFPI2 and are negative for the
markers: ACTC, AGC1, AKR1C1, ALDH1A1, ANXA8, AQP1, AREG, CFB, C3, C20orf103, CCDC3, CD24, CDH3,
CNTNAP2, COMP, COP1, CRYAB, CXADR, DKK2, DLK1, EMID1, FGFR3, GABRB1, GAP43, GDF5, GDF10,
GSC, HOXA5, HSD17B2, HSPA6, HSPB3, ICAM5, ID4, IFI27, INA, KCNMB1, KRT14, KRT17, KRT34, LAMC2,
IGFL3, LOC92196, MFAP5, MEOX1, MEOX2, MMP1, MSX1, MX1, MYBPH, MYH3, MYH11, MYL4, IL32,
NPAS1, NPPB, OLR1, OSR2, PAX2, PAX9, PENK, PITX2, POSTN, PROM1, PRRX1, PTPRN, RASD1, RELN,
RGS1, SMOC1, SMOC2, STMN2, THY1, TNFSF7, TNNT2, TRH, TUBB4, UGT2B7, ZD52F10, ZIC1 and ZIC2.
The cell line MW6 is positive for the markers: BEX1, C6, C7, DIO2, DPT, FOXF1, FST, HTRA3, IGF2, IL1R1,
TMEM119, PITX2, POSTN, PRRX2, SERPINA3, SFRP2, SRCRB4D and SYT12 and are negative for the markers:
AGC1, ALDH1A1, ANXA8, AQP1, ATP8B4, CFB, BMP4, C20orf103, CCDC3, CDH3, CNTNAP2, COP1, CXADR,
DKK2, DLK1, EMID1, FGFR3, TMEM100, GABRB1, GDF10, GSC, HSD11B2, HSD17B2, HSPA6, HSPB3, ID4,
IFI27, IFIT3, INA, KCNMB1, KRT14, KRT17, KRT34, IGFL3, LOC92196, MFAP5, MEOX1, MEOX2, MMP1,
MSX1, MX1, MYH3, MYH11, MYL4, IL32, NLGN4X, NPPB, OLR1, PAX2, PAX9, PENK, PRELP, PROM1, PRRX1,
RARRES1, RASD1, RELN, RGS1, SLITRK6, SMOC1, SMOC2, SNAP25, TAC1, TFPI2, THY1, TNFSF7, TNNT2,
TRH, TSLP, TUBB4, UGT2B7, ZIC1 and ZIC2.
The cell line Q4 is positive for the markers: AREG, BEX1, CRYAB, FMO1, FST, HTRA3, ICAM5, IGF2, IL1R1,
KRT19, TMEM119, PTPRN, SERPINA3, SOD3, SRCRB4D, ZD52F10 and ZIC2 and are negative for the markers:
ACTC, AGC1, ALDH1A1, ANXA8, APCDD1, ATP8B4, CFB, BMP4, C20orf103, CCDC3, CDH3, CDH6, CLDN11,
CNTNAP2, COL15A1, COMP, COP1, DIO2, DKK2, DPT, EGR2, EMID1, FMO3, GAP43, GDF10, GJB2, GSC,
HOXA5, HSD17B2, HSPA6, HSPB3, ID4, IFIT3, INA, KCNMB1, KIAA0644, KRT17, KRT34, IGFL3, LOC92196,
MEOX1, MEOX2, MGP, MMP1, MSX2, MX1, MYBPH, MYH3, MYH11, NLGN4X, NPPB, OGN, OLR1, OSR2,
PAX2, PAX9, PENK, PROM1, PRRX2, PTGS2, RARRES1, RELN, RGMA, RGS1, SLITRK6, SMOC1, SMOC2,
STMN2, SYT12, TAC1, RSPO3, THY1, TNFSF7, TNNT2, TRH, TSLP, TUBB4 and UGT2B7.
The cell line Q6 is positive for the markers: AREG, BEX1, COL21A1, DLK1, FMO1, FST, GDF10, ICAM5, IL1R1,
TMEM119, MYL4, OGN, POSTN, SERPINA3, SFRP2, SOD3, SRCRB4D, ZIC1 and ZIC2 and are negative for the
markers: AGC1, ALDH1A1, ANXA8, AQP1, ATP8B4, CFB, C3, C6, C20orf103, CD24, CDH3, CDH6, CLDN11,
CNTNAP2, COMP, COP1, CXADR, DIO2, DKK2, DPT, EMID1, FGFR3, FMO3, FOXF1, FOXF2, GABRB1, GJB2,
GSC, HOXA5, HSD11B2, HSD17B2, HSPA6, IFI27, INA, KCNMB1, KIAA0644, KRT17, KRT19, KRT34, IGFL3,
LOC92196, MFAP5, MASP1, MEOX1, MEOX2, MMP1, MX1, MYBPH, MYH3, MYH11, IL32, NLGN4X, NPPB,
OLR1, OSR2, PAX2, PAX9, PENK, PITX2, PRELP, PROM1, PTN, PTPRN, RARRES1, RASD1, RELN, RGS1,
SMOC1, SMOC2, SYT12, TAC1, TFPI2, RSPO3, THY1, TNFSF7, TNNT2, TRH, TUBB4 and WISP2.
The cell line Q7 is positive for the markers: AREG, BEX1, COL15A1, COL21A1, COMP, EGR2, FST, GDF10,
HSD17B2, IGF2, SERPINA3, ZIC1 and ZIC2 and is negative for the markers: ACTC, AGC1, AKR1C1, ALDH1A1,
AQP1, ATP8B4, CFB, C3, C6, C7, PRSS35, C20orf103, CCDC3, CD24, CDH3, CLDN11, CNTNAP2, DIO2, DKK2,
DLK1, EMID1, FGFR3, TMEM100, FMO1, FMO3, GABRB1, GDF5, GJB2, GSC, HOXA5, HSD11B2, HSPA6,
HSPB3, ID4, IFI27, KCNMB1, KIAA0644, KRT14, KRT17, KRT34, IGFL3, LOC92196, MFAP5, MASP1, MEOX1,
MEOX2, MGP, MMP1, MSX2, MX1, MYBPH, MYH3, MYH11, IL32, NLGN4X, NPAS1, NPPB, OGN, OLR1, OSR2,
PAX2, PAX9, PDE1A, PENK, PITX2, PODN, POSTN, PRELP, PROM1, PRRX2, PTGS2, PTN, RARRES1, RASD1,
RELN, RGMA, RGS1, SLITRK6, SMOC2, SNAP25, STMN2, TAC1, RSPO3, THY1, TNFSF7, TNNT2, TRH, TUBB4,
UGT2B7 and WISP2.
The cell line RAD20.16 is positive for the markers: ACTC, CD24, CRIP1, CRYAB, FST, HOXA5, HTRA3, KRT19,
LAMC2, MFAP5, MASP1, MGP, MMP1, MSX1, POSTN, S100A4, SRCRB4D and THY1 and is negative for the
markers: AGC1, ALDH1A1, AQP1, AREG, ATP8B4, CFB, C6, C7, C20orf103, CCDC3, CDH3, CLDN11, CNTNAP2,
COL15A1, COL21A1, CRLF1, DLK1, DPT, TMEM100, FMO1, FMO3, FOXF2, GABRB1, GDF10, GJB2, GSC,
HSD11B2, HSD17B2, HSPA6, HSPB3, IFI27, IGF2, KCNMB1, KRT14, TMEM119, IGFL3, LOC92196, MEOX1,
MEOX2, MSX2, MX1, MYH3, MYH11, NLGN4X, NPPB, OGN, OSR2, PAX2, PAX9, PDE1A, PENK, PRG4,
PROM1, PRRX1, RARRES1, RASD1, RGS1, SFRP2, SMOC1, SMOC2, SOD3, STMN2, TAC1, TFPI2, RSPO3, TRH,
TSLP, TUBB4, UGT2B7, WISP2, ZIC1 and ZIC2.
The cell line RAD20.19 is positive for the markers: ACTC, BEX1, CD24, CRIP1, CRYAB, FST, HOXA5, INA, KRT19,
KRT34, LAMC2, MFAP5, MASP1, MMP1, MSX1, NPPB, PTPRN and THY1 and is negative for the markers: AGC1,
ALDH1A1, APCDD1, AQP1, AREG, ATP8B4, CFB, C6, C7, C20orf103, CDH3, CNTNAP2, COL15A1, COL21A1,
COP1, CRLF1, DIO2, METTL7A, DKK2, DLK1, DPT, EGR2, EMID1, TMEM100, FMO1, FMO3, FOXF2, GABRB1,
GDF10, GJB2, GSC, HSD11B2, HSD17B2, HSPA6, HSPB3, ID4, IFI27, IGF2, KIAA0644, KRT14, KRT17, IGFL3,
LOC92196, MEOX1, MEOX2, MGP, MX1, MYBPH, MYH3, NLGN4X, OGN, OSR2, PAX2, PAX9, PDE1A, PENK,
PROM1, PRRX1, PTN, RARRES1, RASD1, RGMA, RGS1, SFRP2, SLITRK6, SMOC1, SMOC2, SNAP25, STMN2,
SYT12, TAC1, RSPO3, TNFSF7, TRH, TSLP, TUBB4, WISP2, ZIC1 and ZIC2.
The cell line RAD20.5 is positive for the markers: AKR1C1, CRIP1, METTL7A, FOXF1, HOXA5, HTRA3, KIAA0644,
KRT19, MASP1, MMP1, MSX1, POSTN, PTPRN, S100A4, SRCRB4D and THY1 and is negative for the markers:
AGC1, ALDH1A1, ANXA8, APCDD1, AQP1, AREG, ATP8B4, BEX1, CFB, C6, C7, PRSS35, C20orf103, CCDC3,
CDH3, CLDN11, CNTNAP2, COL15A1, COL21A1, COMP, CRLF1, CNTNAP2, DKK2, DLK1, DPT, EGR2, EMID1,
TMEM100, FMO1, FMO3, FOXF2, GAP43, GDF10, GSC, HSD11B2, HSD17B2, HSPA6, HSPB3, ID4, IGF2,
KCNMB1, KRT14, KRT34, IGFL3, LOC92196, MEOX1, MEOX2, MGP, MSX2, MYBPH, MYH3, MYH11, MYL4,
IL32, NLGN4X, NPAS1, NPPB, OGN, PAX2, PAX9, PDE1A, PENK, PRELP, PRG4, PROM1, RARRES1, RGMA,
RGS1, SFRP2, SLITRK6, SMOC1, SMOC2, SOD3, STMN2, SYT12, TAC1, TRH, TSLP, TUBB4, UGT2B7, WISP2,
ZIC1 and ZIC2.
The cell line RAPEND17 is positive for the markers: ANXA8, BEX1, C3, CD24, CRIP1, CRYAB, METTL7A, FST,
HOXA5, HTRA3, ICAM5, IFIT3, IGF2, IL1R1, KRT19, LAMC2, MFAP5, MASP1, OLR1, POSTN, PTN, PTPRN and
TFPI2 and is negative for the markers: ACTC, AGC1, APCDD1, AQP1, ATP8B4, CFB, C6, C7, PRSS35, C20orf103,
CCDC3, CDH3, CDH6, CLDN11, CNTNAP2, COL15A1, COL21A1, DKK2, DLK1, DPT, EGR2, EMID1, TMEM100,
FMO1, FMO3, FOXF2, GABRB1, GAP43, GDF10, GSC, HSD11B2, HSD17B2, HSPA6, HSPB3, ID4, KCNMB1,
KRT14, KRT17, IGFL3, LOC92196, MEOX1, MEOX2, MGP, MSX2, MYH3, MYH11, NLGN4X, OGN, OSR2,
PAX2, PAX9, PDE1A, PENK, PRELP, PROM1, PRRX1, PRRX2, RARRES1, RELN, RGMA, RGS1, SFRP2,
SLITRK6, SMOC1, SMOC2, SOD3, SYT12, TAC1, RSPO3, THY1, TNFSF7, TNNT2, TRH, TSLP, TUBB4, UGT2B7,
WISP2, ZD52F10, ZIC1 and ZIC2.
The cell line RASKEL18 is positive for the markers: AREG, CD24, CRYAB, METTL7A, DPT, FST, GJB2, HTRA3,
IGF2, IGFBP5, IL1R1, PTN, PTPRN, SERPINA3, SOX11, SRCRB4D and RSPO3 and is negative for the markers:
ACTC, AKR1C1, ALDH1A1, ANXA8, AQP1, CFB, C7, PRSS35, C20orf103, CDH6, CLDN11, CNTNAP2, COMP,
COP1, DIO2, DKK2, DLK1, EGR2, EMID1, FGFR3, FMO1, FMO3, GAP43, GDF10, GSC, HSD11B2, HSD17B2,
HSPA6, HSPB3, IFI27, INA, KCNMB1, KRT14, KRT17, KRT34, TMEM119, IGFL3, LOC92196, MFAP5, MASP1,
MEOX1, MEOX2, MMP1, MSX2, MYBPH, MYH3, MYH11, MYL4, IL32, NLGN4X, TAGLN3, NPAS1, NPPB,
OGN, OLR1, PAX2, PAX9, PENK, PRELP, PRG4, PROM1, PRRX1, PRRX2, PTGS2, RARRES1, RASD1, RELN,
RGMA, RGS1, SLITRK6, SMOC1, SMOC2, STMN2, SYT12, TAC1, TFPI2, THY1, TNFSF7, TNNT2, TRH, TSLP,
TUBB4, WISP2, ZIC1 and ZIC2.
The cell line RASKEL6 is positive for the markers: AREG, BEX1, C3, CRLF1, CRYAB, METTL7A, FST, HTRA3,
IGF2, IL1R1, TMEM119, PITX2, SERPINA3 and TFPI2 and is negative for the markers: ACTC, AKR1C1, ALDH1A1,
ANXA8, AQP1, CFB, BMP4, C6, CCDC3, CDH3, CDH6, CLDN11, CNTNAP2, COL15A1, COMP, COP1, CXADR,
DKK2, DLK1, EGR2, EMID1, FMO1, FMO3, FOXF2, GAP43, GDF10, GSC, HSD17B2, HSPA6, ID4, IFI27, IFIT3,
IGFBP5, INA, KIAA0644, KRT17, KRT34, LAMC2, IGFL3, LOC92196, MFAP5, MASP1, MEOX1, MEOX2, MMP1,
MSX2, MYBPH, MYH3, MYH11, IL32, NLGN4X, TAGLN3, NPAS1, NPPB, OGN, OLR1, OSR2, PAX2, PAX9,
PENK, POSTN, PRELP, PROM1, PRRX1, PRRX2, RARRES1, RELN, RGMA, RGS1, SLITRK6, SMOC1, SMOC2,
STMN2, SYT12, TAC1, RSPO3, THY1, TNFSF7, TRH, TUBB4, UGT2B7, WISP2, ZIC1 and ZIC2.
The cell line RASKEL8 is positive for the markers: AREG, BEX1, C7, CRIP1, CRLF1, CRYAB, FST, HOXA5,
HTRA3, ICAM5, IGF2, IL1R1, KRT19, LAMC2, PITX2, POSTN, PTPRN, SERPINA3 and TFPI2 and is negative for
the markers: ACTC, AGC1, ALDH1A1, AQP1, ATP8B4, CFB, C6, PRSS35, C20orf103, CCDC3, CDH3, CDH6,
CLDN11, CNTNAP2, COMP, COP1, DKK2, DLK1, DPT, EMID1, FMO1, FMO3, FOXF2, GABRB1, GAP43, GDF10,
GSC, HSD11B2, HSD17B2, HSPA6, HSPB3, IFI27, IGFBP5, KCNMB1, KIAA0644, KRT14, KRT17, KRT34, IGFL3,
LOC92196, MEOX1, MEOX2, MGP, MMP1, MSX2, MX1, MYH3, MYH11, NLGN4X, TAGLN3, NPPB, OGN,
OSR2, PAX2, PAX9, PDE1A, PENK, PRELP, PRG4, PROM1, PRRX1, PRRX2, PTN, RARRES1, RELN, RGMA,
RGS1, SFRP2, SLITRK6, SMOC1, SMOC2, SNAP25, STMN2, SYT12, TAC1, RSPO3, TNFSF7, TNNT2, TRH,
TSLP, TUBB4, WISP2, ZIC1 and ZIC2.
The cell line SK1 is positive for the markers: AKR1C1, BEX1, C6, C7, COL21A1, CRIP1, METTL7A, DLK1,
TMEM100, FMO1, FMO3, FOXF2, FST, HSD11B2, HTRA3, ICAM5, IGF2, IL1R1, TMEM119, MGP, MSX1, PRG4,
PTN, PTPRN, S100A4, SERPINA3, SFRP2, SOD3, SOX11, WISP2 and ZIC1 and is negative for the markers: AGC1,
ALDH1A1, ANXA8, AQP1, ATP8B4, BMP4, C20orf103, CD24, CDH3, CDH6, CLDN11, CNTNAP2, COMP, COP1,
CRLF1, DKK2, EGR2, EMID1, FGFR3, GABRB1, GAP43, GDF10, GJB2, GSC, HOXA5, HSD17B2, HSPA6, ID4,
IFI27, IFIT3, INA, KCNMB1, KRT14, KRT17, KRT19, KRT34, LAMC2, IGFL3, LOC92196, MFAP5, MASP1,
MEOX1, MEOX2, MMP1, MSX2, MX1, MYBPH, MYH11, IL32, NLGNHX, NPAS1, NPPB, OLR1, PAX2, PAX9,
PENK, PITX2, POSTN, PRELP, PROM1, RARRES1, RGS1, SMOC2, SYT12, TFPI2, RSPO3, THY1, TNNT2, TRH,
TSLP, TUBB4 and ZIC2.
The group of cell lines SK10Bio1 and SK10Bio2 are positive for the markers: BEX1, COL21A1, FST, ICAM5, IL1R1,
TMEM119, SERPINA3 and ZIC2 and are negative for the markers: ACTC, AGC1, ALDH1A1, AQP1, CFB, BMP4, C3,
C6, C20orf103, CDH3, CLDN11, CNTNAP2, DKK2, DPT, EMID1, TMEM100, FMO3, GABRB1, GAP43, GSC,
HOXA5, HSPA6, ID4, IFI27, KIAA0644, KRT14, KRT34, IGFL3, LOC92196, MFAP5, MEOX1, MEOX2, MX1,
MYBPH, MYH3, MYH11, NLGN4X, NPPB, OLR1, PAX2, PAX9, PDE1A, PENK, PROM1, RARRES1, RASD1,
RELN, RGS1, SLITRK6, SMOC1, SMOC2, STMN2, SYT12, TAC1, RSPO3, THY1, TNNT2 and TUBB4.
The group of cell lines SK11, SK44, SK50 and SK52 are positive for the markers: BEX1, COL21A1, FST, ICAM5,
IL1R1, TMEM119, PTPRN, SERPINA3, SFRP2 and ZIC1 and are negative for the markers: ACTC, AGC1, ALDH1A1,
AQP1, ATP8B4, C6, C20orf103, CCDC3, CDH3, CLDN11, CNTNAP2, DIO2, DKK2, EMID1, GABRB1, GSC,
HOXA5, HSPA6, IFI27, INA, KRT14, KRT34, IGFL3, LOC92196, MEOX1, MEOX2, MMP1, MX1, MYH3, MYH11,
IL32, NLGN4X, NPPB, OLR1, PAX2, PAX9, PDE1A, PENK, PROM1, PTN, RARRES1, RASD1, RELN, RGS1,
SMOC1, SMOC2, STMN2, TAC1, TFPI2, RSPO3, TNFSF7, TNNT2, TRH and TUBB4.
The group of cell lines SK14, SK53, SK60 and SK61 are positive for the markers: C7, COL21A1, CRYAB, HTRA3,
IL1R1, MGP, PTPRN, RGMA, SERPINA3 and SFRP2 and are negative for the markers: ACTC, AGC1, ALDH1A1,
ANXA8, AQP1, ATP8B4, CFB, BMP4, CCDC3, CDH3, CNTNAP2, COP1, CXADR, DKK2, GABRB1, GAP43,
GDF10, GJB2, GSC, HOXA5, HSD17B2, IFI27, IFIT3, KRT14, KRT17, KRT34, IGFL3, LOC92196, MFAP5,
MEOX1, MEOX2, MMP1, MX1, MYBPH, MYH3, MYH11, IL32, NLGN4X, NPPB, OLR1, PAX2, PAX9, PENK,
PROM1, RASD1, RELN, RGS1, SLITRK6, SMOC1, SMOC2, STMN2, TAC1, RSPO3, TNNT2, TRH, TUBB4,
UGT2B7, ZIC1 and ZIC2.
The cell line SK17 is positive for the markers: ACTC, APCDD1, BEX1, COL21A1, METTL7A, DLK1, FST, HOXA5,
HSPB3, HTRA3, IGF2, IL1R1, KIAA0644, MASP1, MGP, MYBPH, MYH3, NLGN4X, PDE1A, PTN, RGMA,
SRCRB4D, STMN2, RSPO3 and TNNT2 and is negative for the markers: AGC1, AKR1C1, ALDH1A1, ANXA8,
AQP1, CFB, C6, C20orf103, CCDC3, CDH3, CDH6, CLDN11, CNTNAP2, COL15A1, COMP, COP1, CRLF1, DKK2,
DPT, TMEM100, FMO1, FMO3, FOXF2, GABRB1, GDF10, GSC, HSD17B2, HSPA6, ID4, IFI27, INA, KCNMB1,
KRT14, KRT34, LAMC2, TMEM119, IGFL3, LOC92196, MFAP5, MEOX1, MEOX2, MMP1, MX1, MYH11, IL32,
NPAS1, NPPB, OLR1, PAX2, PAX9, PENK, PITX2, PRELP, RASD1, RELN, RGS1, S100A4, SLITRK6, SMOC1,
SMOC2, TAC1, THY1, TNFSF7, TRH, TSLP, TUBB4, UGT2B7, WISP2, ZIC1 and ZIC2.
The cell line SK18 is positive for the markers: APCDD1, COL21A1, METTL7A, FMO1, FOXF1, FST, HTRA3, IGF2,
IL1R1, TMEM119, OGN, PITX2, PRRX1, RGMA, SERPINA3, SFRP2, SOD3 and TSLP and is negative for the
markers: ACTC, AGC1, AKR1C1, ALDH1A1, ANXA8, AQP1, AREG, ATP8B4, CFB, BMP4, C3, C6, C7, C20orf103,
CCDC3, CD24, CDH3, CNTNAP2, COP1, CXADR, DIO2, DKK2, DLK1, DPT, EMID1, TMEM100, GABRB1,
GAP43, GDF5, GDF10, GJB2, GSC, HOXA5, HSD17B2, HSPA6, HSPB3, ID4, IFI27, INA, KIAA0644, KRT14,
KRT17, KRT19, KRT34, LAMC2, IGFL3, LOC92196, MFAP5, MEOX1, MEOX2, MMP1, MSX1, MX1, MYBPH,
MYH3, MYH11, MYL4, IL32, NLGN4X, NPPB, OLR1, OSR2, PAX2, PAX9, PDE1A, PENK, PRELP, PROM1,
RARRES1, RASD1, RELN, RGS1, SLITRK6, SMOC1, SMOC2, STMN2, TAC1, TFPI2, RSPO3, THY1, TNFSF7,
TNNT2, TRH, TUBB4, UGT2B7, ZIC1 and ZIC2.
The cell line SK26 is positive for the markers: APCDD1, BEX1, COL21A1, CRYAB, FMO1, FOXF2, FST, HTRA3,
ICAM5, IL1R1, TMEM119, PRRX1, PTPRN, SERPINA3 and SFRP2 and is negative for the markers: ACTC, AGC1,
ALDH1A1, ANXA8, AQP1, AREG, ATP8B4, CFB, BMP4, C3, C6, C7, C20orf103, CCDC3, CD24, CDH3, CLDN11,
CNTNAP2, COP1, CXADR, DKK2, DLK1, DPT, EGR2, EMID1, FGFR3, GABRB1, GAP43, GDF10, GJB2, GSC,
HOXA5, HSD17B2, HSPA6, IFI27, IFIT3, KIAA0644, KRT14, KRT17, KRT34, IGFL3, LOC92196, MFAP5, MEOX1,
MEOX2, MMP1, MX1, MYBPH, MYH3, MYH11, MYL4, IL32, NLGN4X, NPPB, OLR1, OSR2, PAX2, PAX9,
PDE1A, PENK, PITX2, POSTN, PROM1, PTN, RARRES1, RASD1, RELN, RGS1, SLITRK6, SMOC1, SMOC2,
SNAP25, STMN2, TAC1, TFPI2, RSPO3, THY1, TNFSF7, TNNT2, TRH, TUBB4, UGT2B7 and ZIC1.
The group of cell lines SK27 and T7 are positive for the markers: BEX1, PRSS35, CCDC3, CDH6, COL21A1, CRIP1,
CRYAB, GAP43, IGF2, KRT19, LAMC2, POSTN, S100A4, SFRP2, SOX11 and ZIC2 and are negative for the markers:
AGC1, ALDH1A1, APCDD1, AREG, ATP8B4, CFB, C3, C7, C20orf103, CDH3, CLDN11, CNTNAP2, COP1,
CXADR, DLK1, DPT, EGR2, EMID1, GDF10, GJB2, GSC, HOXA5, HSD11B2, HSD17B2, HSPA6, HSPB3, IFI27,
INA, KRT14, IGFL3, LOC92196, MFAP5, MEOX1, MEOX2, MMP1, MYBPH, MYH3, MYL4, NLGN4X, NPPB,
OLR1, PAX2, PAX9, PDE1A, PENK, PRG4, PROM1, RARRES1, RASD1, RELN, RGS1, SLITRK6, SMOC1,
SMOC2, SNAP25, STMN2, TAC1, TFPI2, RSPO3, TNNT2, TRH, TUBB4 and ZIC1.
The group of cell lines SK28 and SK57 are positive for the markers: BEX1, COL21A1, CRYAB, HTRA3, ICAM5,
IGF2, IL1R1, PTPRN and SERPINA3 and are negative for the markers: AGC1, ALDH1A1, AQP1, ATP8B4, CFB,
BMP4, C20orf103, CCDC3, CDH3, CDH6, CLDN11, CNTNAP2, COP1, CXADR, DIO2, DKK2, EMID1, GABRB1,
GAP43, GDF10, GSC, HOXA5, HSD17B2, HSPA6, HSPB3, ID4, IFI27, KCNMB1, KIAA0644, KRT14, KRT17,
KRT34, IGFL3, LOC92196, MFAP5, MEOX1, MEOX2, MMP1, MSX2, MX1, MYH3, MYH11, MYL4, IL32,
NLGN4X, NPPB, OLR1, OSR2, PAX2, PAX9, PENK, PROM1, PTN, RARRES1, RASD1, RELN, RGS1, SLITRK6,
SMOC1, SMOC2, STMN2, TAC1, TFPI2, RSPO3, TNFSF7, TNNT2, TRH, TUBB4 and UGT2B7.
The group of cell lines SK30 and W4 are positive for the markers: BEX1, FST, HTRA3, IGF2, TMEM119, POSTN,
SOX11, SRCRB4D, ZIC1 and ZIC2 and are negative for the markers: AGC1, ALDH1A1, ANXA8, AQP1, ATP8B4, C3,
C6, C7, C20orf103, CCDC3, CDH3, CLDN11, CRYAB, DIO2, METTL7A, EGR2, EMID1, FMO3, FOXF2, GABRB1,
GSC, HOXA5, HSD11B2, HSPA6, HSPB3, ID4, IFI27, INA, KRT14, KRT17, KRT34, IGFL3, LOC92196, MFAP5,
MASP1, MEOX1, MBOX2, MMP1, MX1, MYH3, MYH11, NPPB, OLR1, OSR2, PAX2, PAX9, PDE1A, PENK,
PRELP, PROM1, RARRES1, RASD1, RELN, SMOC2, STMN2, SYT12, TAC1, RSPO3, TNFSF7, TNNT2 and
TUBB4.
The group of cell lines SK31 and SK54 are positive for the markers: BEX1, COL21A1, CRIP1, CRYAB, TMEM100,
FMO1, FMO3, FOXF1, FOXF2, IGF2, IGFBP5, IL1R1, KRT19, LAMC2, TMEM119, NPAS1, PDE1A, PRRX2,
S100A4, SERPINA3, SNAP25, SOX11, SRCRB4D and WISP2 and are negative for the markers: ACTC, AGC1,
AKR1C1, ALDH1A1, ANXA8, AQP1, AREG, ATP8B4, CFB, BMP4, C3, CCDC3, CD24, CDH3, CLDN11,
CNTNAP2, COMP, COP1, CXADR, DKK2, DLK1, DPT, EMID1, FGFR3, GABRB1, GAP43, GDF10, GSC,
HSD17B2, HSPA6, HTRA3, ID4, IFI27, INA, KCNMB1, KRT14, KRT17, KRT34, IGFL3, LOC92196, MFAP5,
MASP1, MEOX1, MEOX2, MMP1, MYH3, MYH11, MYL4, IL32, NLGN4X, NPPB, OGN, OLR1, OSR2, PAX2,
PAX9, PENK, PITX2, PRELP, PROM1, PRRX1, RELN, RGS1, SLITRK6, SMOC1, SMOC2, SOD3, STMN2, SYT12,
TAC1, TFPI2, RSPO3, TNFSF7, TNNT2, TRH, TSLP, TUBB4, ZIC1 and ZIC2.
The cell line SK32 is positive for the markers: AKR1C1, BEX1, C6, C7, C20orf103, COL21A1, CRYAB, METTL7A,
DPT, GDF5, HTRA3, ICAM5, IL1R1, TMEM119, MGP, OGN, POSTN, PTPRN, RGMA, SERPINA3, SFRP2, SOD3,
WISP2 and ZIC1 and is negative for the markers: ACTC, AGC1, ALDH1A1, ANXA8, AQP1, AREG, ATP8B4, CFB,
BMP4, C3, CCDC3, CD24, CDH3, CDH6, CLDN11, CNTNAP2, COL15A1, COMP, COP1, CXADR, DIO2, DKK2,
EGR2, EMID1, FGFR3, FMO3, FOXF1, FOXF2, GABRB1, GAP43, GDF10, GSC, HOXA5, HSD17B2, HSPA6,
HSPB3, ID4, IFI27, IFIT3, INA, KIAA0644, KRT14, KRT17, KRT19, KRT34, IGFL3, LOC92196, MFAP5, MASP1,
MEOX1, MEOX2, MMP1, MX1, MYBPH, MYH3, MYH11, MYL4, IL32, NLGN4X, NPPB, OLR1, OSR2, PAX2,
PAX9, PENK, PITX2, PRELP, PROM1, PTGS2, RASD1, RELN, RGS1, SLITRK6, SMOC1, SMOC2, STMN2,
SYT12, TFPI2, RSPO3, THY1, TNFSF7, TNNT2, TRH, TSLP, TUBB4 and ZIC2.
The group of cell lines SK40 and SK40Bio2 are positive for the markers: BEX1, COL21A1, CRYAB, FMO1, FST,
ICAM5, IGFBP5, TMEM119, MSX1, MYL4, PTPRN, SERPINA3, SOD3, ZIC1 and ZIC2 and are negative for the
markers: AGC1, AKR1C1, ALDH1A1, AQP1, ATP8B4, BMP4, C3, C20orf103, CCDC3, CD24, CDH3, CLDN11,
CNTNAP2, COP1, DIO2, DKK2, DPT, TMEM100, FMO3, GABRB1, GAP43, GSC, HOXA5, HSPA6, HSPB3, ID4,
IFI27, INA, KCNMB1, KIAA0644, KRT14, KRT17, KRT34, IGFL3, LOC92196, MEOX1, MEOX2, MX1, MYBPH,
MYH11, NLGN4X, NPPB, OGN, OLR1, PAX2, PAX9, PDE1A, PENK, PITX2, PRELP, PROM1, RARRES1, RASD1,
RELN, RGS1, SMOC2, SNAP25, SYT12, TAC1, TFPI2, RSPO3, THY1, TNFSF7, TRH, TSLP and TUBB4
The cell line SK46 is positive for the markers: APCDD1, COL21A1, DIO2, METTL7A, FMO1, FMO3, FOXF1, FOXF2,
FST, HTRA3, IGF2, IL1R1, TMEM119, OGN, PRRX1, PRRX2, SERPINA3, SFRP2, SLITRK6, TSLP and ZIC2 and is
negative for the markers: ACTC, AGC1, ALDH1A1, ANXA8, AQP1, ATP8B4, CFB, BMP4, C3, C6, C7, C20orf103,
CCDC3, CD24, CDH3, CLDN11, CNTNAP2, COP1, CRIP1, CXADR, DKK2, DPT, EMID1, FGFR3, GABRB1,
GAP43, GDF5, GDF10, GJB2, GSC, HOXA5, HSD17B2, HSPA6, HSPB3, IFI27, INA, KRT14, KRT17, KRT19,
KRT34, LAMC2, IGFL3, LOC92196, MFAP5, MEOX1, MEOX2, MMP1, MX1, MYBPH, MYH3, MYH11, MYL4,
IL32, NLGN4X, TAGLN3, NPAS1, NPPB, OLR1, OSR2, PAX2, PAX9, PDE1A, PENK, PITX2, POSTN, PRELP,
PROM1, RARRES1, RASD1, RELN, RGS1, SMOC1, SMOC2, STMN2, TFPI2, RSPO3, THY1, TNFSF7, TNNT2,
TRH, TUBB4, UGT2B7 and ZIC1.
The cell line SK47 is positive for the markers: BEX1, COL21A1, METTL7A, FMO1, FOXF1, FOXF2, FST, HTRA3,
ICAM5, IGF2, IL1R1, KRT19, TMEM119, MSX1, PRRX2, PTPRN, SERPINA3, SOD3 and ZIC1 and is negative for
the markers: AGC1, ALDH1A1, AQP1, ATP8B4, CFB, BMP4, C3, C6, C7, C20orf103, CCDC3, CD24, CDH3,
CLDN11, CNTNAP2, COL15A1, COP1, CRLF1, DKK2, DPT, EGR2, EMID1, FGFR3, GABRB1, GAP43, GDF10,
GJB2, GSC, HOXA5, HSD17B2, HSPA6, HSPB3, ID4, IFI27, INA, KCNMB1, KRT14, KRT17, KRT34, IGFL3,
LOC92196, MFAP5, MEOX1, MEOX2, MGP, MMP1, MX1, MYBPH, MYH3, MYH11, IL32, NLGN4X, NPPB,
OLR1, PAX2, PAX9, PDE1A, PENK, PITX2, POSTN, PRELP, PROM1, RARRES1, RASD1, RELN, RGS1, SLITRK6,
SMOC1, SMOC2, STMN2, SYT12, TAC1, TFPI2, RSPO3, THY1, TNFSF7, TNNT2, TRH, TUBB4 and ZD52F10.
The group of cell lines SK5.Bio1, SK5.Bio2, SK5Bio3 and SK5BioUT are positive for the markers: ACTC, C7, CRLF1,
CRYAB, FST, HTRA3, IL1R1, TMEM119, MGP, PTPRN, SERPINA3, SFRP2 and ZIC1 and are negative for the
markers: ALDH1A1, ANXA8, CFB, BMP4, C3, C20orf103, CDH3, CLDN11, CNTNAP2, COP1, DKK2, EMID1,
FMO3, GABRB1, GDF10, GSC, HSD17B2, HSPB3, IFI27, KRT14, KRT17, KRT34, IGFL3, LOC92196, MFAP5,
MEOX1, MEOX2, MYH11, IL32, NPPB, OLR1, OSR2, PAX2, PAX9, PENK, PRELP, PROM1, RARRES1, RELN,
RGS1, SLITRK6, SMOC1, SMOC2, STMN2, RSPO3, TNFSF7, TNNT2, TRH, TUBB4 and ZIC2.
The cell line SK8 is positive for the markers: APCDD1, BEX1, COL21A1, CRLF1, FMO1, FMO3, FOXF2, FST,
HTRA3, ICAM5, IGF2, IL1R1, TMEM119, MASP1, PTPRN, SERPINA3 and SFRP2 and is negative for the markers:
ACTC, AGC1, ALDH1A1, ANXA8, AQP1, ATP8B4, CFB, BMP4, C7, PRSS35, C20orf103, CD24, CDH3, CDH6,
CLDN11, CNTNAP2, COP1, DKK2, EMID1, GABRB1, GAP43, GDF10, GJB2, GSC, HOXA5, HSD17B2, HSPA6,
HSPB3, IFI27, IFIT3, INA, KIAA0644, KRT14, KRT17, KRT34, IGFL3, LOC92196, MFAP5, MEOX1, MEOX2,
MMP1, MX1, MYBPH, MYH3, MYH11, MYL4, IL32, NLGN4X, NPPB, OLR1, OSR2, PAX2, PAX9, PDE1A, PENK,
PRELP, PROM1, PTN, RARRES1, RASD1, RELN, RGS1, SMOC1, SMOC2, STMN2, TAC1, RSPO3, THY1,
TNFSF7, TNNT2, TRH, TUBB4, ZIC1 and ZIC2.
The cell line SM17 is positive for the markers: BEX1, CD24, CRYAB, EGR2, FOXF1, FST, GDF5, HTRA3, IGFBP5,
KRT19, MMP1, MSX1, MSX2, IL32, PODN, POSTN, PRELP, PRRX2, SRCRB4D, TFPI2, TSLP and ZIC1 and is
negative for the markers: AGC1, AKR1C1, ALDH1A1, ANXA8, APCDD1, AQP1, AREG, ATP8B4, CFB, BMP4, C6,
C7, C20orf103, CCDC3, CDH3, CLDN11, CNTNAP2, COL15A1, DIO2, METTL7A, DKK2, DLK1, DPT, FGFR3,
TMEM100, FMO1, FMO3, GABRB1, GAP43, GDF10, GSC, HOXA5, HSD11B2, HSD17B2, HSPA6, IFI27, IGF2,
KCNMB1, KRT14, KRT17, KRT34, IGFL3, LOC92196, MFAP5, MEOX1, MEOX2, MYBPH, MYH3, MYH11,
NLGN4X, NPPB, OLR1, OSR2, PAX2, PAX9, PDE1A, PENK, PRG4, PROM1, RARRES1, RASD1, RELN, RGS1,
SMOC1, SMOC2, SNAP25, STMN2, TAC1, RSPO3, TNFSF7, TNNT2, TRH, TUBB4, UGT2B7, WISP2 and ZIC2.
The cell line SM19 is positive for the markers: BEX1, CNTNAP2, CRYAB, FST, GDF5, MMP1, POSTN, PRRX2,
SERPINA3 and SFRP2 and is negative for the markers: ACTC, AGC1, AKR1C1, ALDH1A1, ANXA8, AQP1, AREG,
ATP8B4, CFB, BMP4, C3, C6, C7, C20orf103, CDH3, CDH6, CLDN11, COL21A1, COMP, COP1, CRLF1, DIO2,
METTL7A, DKK2, DLK1, DPT, EMID1, FGFR3, TMEM100, FMO1, FMO3, FOXF2, GABRB1, GDF10, GSC,
HOXA5, HSD11B2, HSD17B2, HSPA6, ID4, IFI27, IGF2, IGFBP5, IL1R1, KCNMB1, KIAA0644, KRT14, KRT17,
KRT34, IGFL3, LOC92196, MFAP5, MASP1, MEOX1, MEOX2, MGP, MX1, MYBPH, MYH3, MYH11, NLGN4X,
NPPB, OGN, OLR1, OSR2, PAX2, PAX9, PDE1A, PENK, PITX2, PRG4, PROM1, RARRES1, RASD1, RGS1,
SLITRK6, SMOC1, SMOC2, SNAP25, STMN2, SYT12, TAC1, TFPI2, RSPO3, THY1, TNFSF7, TNNT2, TRH,
UGT2B7, WISP2, ZIC1 and ZIC2.
The cell line SM2 is positive for the markers: CDH6, CNTNAP2, COL15A1, COL21A1, FST, GDF5, TMEM119,
MMP1, MSX1, POSTN, PRRX1, SOD3, ZIC1 and ZIC2 and is negative for the markers: ACTC, AGC1, AKR1C1,
ALDH1A1, ANXA8, APCDD1, AQP1, AREG, ATP8B4, BEX1, BMP4, C3, C6, C7, PRSS35, C20orf103, CCDC3,
CD24, CDH3, CLDN11, COMP, CRIP1, CRYAB, DIO2, DPT, EMID1, FGFR3, TMEM100, FMO3, GDF10, GJB2,
GSC, HOXA5, HSD11B2, HSD17B2, HSPA6, HSPB3, ID4, IFI27, INA, KCNMB1, KIAA0644, KRT14, KRT17,
KRT19, KRT34, IGFL3, LOC92196, MFAP5, MASP1, MEOX1, MEOX2, MGP, MX1, MYBPH, MYH3, MYH11,
MYL4, IL32, NLGN4X, NPAS1, NPPB, OLR1, OSR2, PAX2, PAX9, PDE1A, PENK, PITX2, PROM1, RARRES1,
RASD1, RELN, RGS1, SFRP2, SLITRK6, SMOC1, SMOC2, STMN2, SYT12, TAC1, TFPI2, RSPO3, TNFSF7,
TNNT2, TRH, TUBB4 and UGT2B7.
The cell line SM22 is positive for the markers: CDH6, CRLF1, DLK1, FOXF1, FST, GDF5, HTRA3, IGFBP5, IL1R1,
MGP, MMP1, MSX1, MSX2, OGN, POSTN, PRRX2, PTN, RGMA, SOD3, SRCRB4D, STMN2, TSLP, ZD52F10 and
ZIC1 and is negative for the markers: AGC1, ALDH1A1, ANXA8, APCDD1, AQP1, AREG, BMP4, C3, C6, C7,
C20orf103, CCDC3, CDH3, CLDN11, CNTNAP2, COL15A1, CRIP1, CXADR, DIO2, DKK2, DPT, TMEM100,
FMO1, FOXF2, GDF10, GJB2, GSC, HOXA5, HSD11B2, HSD17B2, HSPA6, HSPB3, ICAM5, IFI27, INA, KRTI4,
KRT17, KRT34, LAMC2, TMEM119, IGFL3, LOC92196, MFAP5, MASP1, MEOX1, MEOX2, MX1, MYBPH,
MYH3, MYH11, MYL4, IL32, NLGN4X, NPAS1, NPPB, OLR1, OSR2, PAX2, PAX9, PENK, PITX2, PRG4, PROM1,
PTPRN, RARRES1, RASD1, RELN, RGS1, SFRP2, SMOC1, SMOC2, SNAP25, TAC1, RSPO3, TNFSF7, TNNT2,
TRH, TUBB4, UGT2B7 and ZIC2.
The group of cell lines SM25 and Z8 are positive for the markers: FOXF1, FST, GDF5, HTRA3, MSX1, MSX2, PRRX2
and SRCRB4D and are negative for the markers: ACTC, AGC1, AKR1C1, ALDH1A1, ANXA8, AQP1, AREG,
ATP8B4, BMP4, C6, C7, C20orf103, CD24, CDH3, CLDN11, CNTNAP2, METTL7A, DKK2, EMID1, TMEM100,
FMO1, GABRB1, GDF10, GSC, HOXA5, HSD11B2, HSD17B2, HSPA6, ID4, IFI27, KCNMB1, KRT14, KRT17,
KRT34, IGFL3, LOC92196, MFAP5, MEOX1, MEOX2, MYBPH, MYH3, MYH11, MYL4, NLGN4X, NPPB, OLR1,
OSR2, PAX2, PAX9, PDE1A, PENK, PITX2, PROM1, RARRES1, RASD1, RGS1, RPS4Y2, SFRP2, SLITRK6,
SMOC1, SMOC2, TAC1, RSPO3, TNFSF7, TNNT2, TRH, TUBB4 and UGT2B7.
The cell line SM28 is positive for the markers: COMP, CRLF1, DIO2, EGR2, FOXF1, FOXF2, FST, HSPB3, INA,
TMEM119, MGP, MMP1, MSX2, POSTN, PRELP, PRRX2, PTN and SYT12 and is negative for the markers: ACTC,
AGC1, AKR1C1, ALDH1A1, ANXA8, APCDD1, AQP1, AREG, ATP8B4, BEX1, CFB, C3, C6, C7, C20orf103, CD24,
CDH6, CLDN11, CNTNAP2, COL21A1, CXADR, METTL7A, DKK2, DLK1, FGFR3, TMEM100, FMO1, GABRB1,
GAP43, GDF10, GJB2, GSC, HOXA5, HSD11B2, HSD17B2, HSPA6, ID4, IFI27, IFIT3, KCNMB1, KRT14, KRT17,
KRT19, KRT34, LAMC2, IGFL3, LOC92196, MFAP5, MEOX1, MEOX2, MX1, MYBPH, MYH3, MYH11, IL32,
NLGN4X, TAGLN3, NPPB, OGN, OLR1, OSR2, PAX2, PAX9, PDE1A, PENK, PITX2, PRG4, PROM1, PTGS2,
PTPRN, RARRES1, RASD1, RGS1, RPS4Y2, SERPINA3, SFRP2, SMOC1, SMOC2, SNAP25, STMN2, TAC1,
RSPO3, TNFSF7, TNNT2, TRH, TUBB4, UGT2B7, WISP2, ZD52F10, ZIC1 and ZIC2.
The cell line SM29 is positive for the markers: FOXF1, FOXF2, FST, HTRA3, IGF2, IGFBP5, IL1R1, MASP1, MGP,
MMP1, MSX2, OGN, PODN, POSTN, PRELP, PRRX2, PTN, SRCRB4D and TSLP and is negative for the markers:
ACTC, AKR1C1, ALDH1A1, ANXA8, APCDD1, AQP1, CFB, C6, C7, CCDC3, CDH3, CLDN11, CNTNAP2,
COL15A1, COL21A1, CRIP1, CRLF1, CRYAB, DKK2, DPT, FGFR3, TMEM100, GDF10, GSC, HOXA5, HSD11B2,
HSD17B2, HSPA6, ID4, IFI27, INA, KCNMB1, KRT14, KRT17, KRT34, LAMC2, IGFL3, LOC92196, MFAP5,
MEOX1, MEOX2, MX1, MYBPH, MYH3, MYH11, MYL4, IL32, NLGN4X, NPPB, OLR1, OSR2, PAX9, PDE1A,
PENK, PITX2, PROM1, RARRES1, RASD1, RELN, RGS1, S100A4, SMOC1, SMOC2, SNAP25, TAC1, RSPO3,
TNFSF7, TNNT2, TRH, TUBB4, UGT2B7, WISP2, ZIC1 and ZIC2.
The cell line SM30 is positive for the markers: COL15A1, CRYAB, DYSF, FST, GDF5, HTRA3, TMEM119, MMP1,
MSX1, MSX2, MYL4, POSTN, SERPINA3, SRCRB4D and ZIC2 and is negative for the markers: ACTC, AGC1,
AKR1C1, ALDH1A1, ANXA8, APCDD1, AQP1, ATP8B4, CFB, C3, C6, C7, C20orf103, CD24, CDH3, CLDN11,
CNTNAP2, COMP, DIO2, METTL7A, DKK2, DLK1, DPT, FGFR3, TMEM100, FMO1, FMO3, FOXF2, GABRB1,
GJB2, GSC, HOXA5, HSD11B2, HSPA6, ID4, IFI27, IL1R1, KCNMB1, KIAA0644, KRT14, KRT17, KRT34, IGFL3,
LOC92196, MEOX1, MEOX2, MGP, MYBPH, MYH3, MYH11, NLGN4X, NPPB, OGN, OLR1, OSR2, PAX2, PAX9,
PDE1A, PENK, PRG4, PROM1, PRRX1, PTN, RARRES1, RASD1, RELN, RGS1, SLITRK6, SMOC1, SMOC2,
SNAP25, STMN2, TAC1, RSPO3, TNFSF7, TNNT2, TRH, TUBB4, UGT2B7 and WISP2.
The cell line SM33 is positive for the markers: BEX1, CDH6, CRLF1, EGR2, FOXF1, FST, IGFBP5, MSX1, MSX2,
PRELP, SERPINA3, SRCRB4D, SYT12, TSLP and ZIC2 and is negative for the markers: ACTC, AGC1, AKR1C1,
ALDH1A1, ANXA8, APCDD1, AQP1, AREG, ATP8B4, CFB, BMP4, C3, C6, C7, C20orf103, CD24, CDH3, CLDN11,
CNTNAP2, COL21A1, CRIP1, DIO2, METTL7A, DLK1, DPT, EMID1, FGFR3, TMEM100, FMO1, GABRB1,
GAP43, GSC, HOXA5, HSD11B2, HSPA6, HSPB3, ID4, IFI27, IL1R1, KCNMB1, KRT14, KRT17, KRT34, IGFL3,
LOC92196, MFAP5, MEOX1, MEOX2, MX1, MYBPH, MYH3, MYH11, NLGN4X, NPPB, OGN, OSR2, PAX2,
PAX9, PDE1A, PENK, PRG4, PROM1, PTGS2, RARRES1, RASD1, RELN, RGS1, RPS4Y2, SFRP2, SMOC1,
SMOC2, SNAP25, STMN2, TAC1, RSPO3, THY1, TNFSF7, TRH, TUBB4, UGT2B7, WISP2 and ZIC1.
The cell line SM4 is positive for the markers: BEX1, CCDC3, CDH6, CRLF1, EGR2, FST, GABRB1, GAP43, GDF5,
HSPB3, HTRA3, MMP1, MSX1, MSX2, PRELP, PRRX1, PRRX2 and SRCRB4D and is negative for the markers:
AGC1, ALDH1A1, ANXA8, APCDD1, AQP1, AREG, ATP8B4, CFB, BMP4, C3, C6, C7, PRSS35, C20orf103, CD24,
CDH3, CLDN11, CNTNAP2, COL15A1, COL21A1, COP1, CXADR, METTL7A, DKK2, DLK1, DPT, EMID1,
FGFR3, TMEM100, FMO1, FMO3, GDF10, GJB2, GSC, HOXA5, HSD11B2, HSD17B2, HSPA6, ICAM5, ID4, IFI27,
IGF2, KRT14, KRT17, KRT19, KRT34, IGFL3, LOC92196, MFAP5, MASP1, MEOX1, MEOX2, MX1, MYBPH,
MYH3, MYH11, MYL4, IL32, NLGN4X, TAGLN3, NPAS1, NPPB, OLR1, OSR2, PAX2, PAX9, PDE1A, PENK,
PITXZ, PRG4, PROM1, RARRES1, RASD1, RELN, RGS1, SFRP2, SLITRK6, SMOC1, SMOC2, SNAP25, STMN2,
TAC1, RSPO3, TNFSF7, TNNT2, TRH, TSLP, TUBB4, UGT2B7, WISP2, ZD52F10 and ZIC1.
The cell line SM40 is positive for the markers: BEX1, CD24, CRYAB, FST, HSPB3, IGFBP5, KRT19, MMP1, MYL4,
POSTN, PRELP, SRCRB4D and ZD52F10 and is negative for the markers: AGC1, AKR1C1, ALDH1A1, ANXA8,
APCDD1, AQP1, AREG, CFB, C6, C7, CDH3, CDH6, CLDN11, CNTNAP2, COL15A1, COL21A1, COMP, CRLF1,
DIO2, METTL7A, DKK2, DLK1, DPT, EMID1, FGFR3, TMEM100, FMO1, FMO3, GABRB1, GAP43, GDF10, GJB2,
GSC, HOXA5, HSD11B2, HSD17B2, HSPA6, ID4, IFI27, IGF2, KRT14, KRT17, KRT34, IGFL3, LOC92196,
MEOX1, MEOX2, MGP, MX1, MYBPH, MYH3, MYH11, NLGN4X, NPPB, OGN, OSR2, PAX2, PAX9, PDE1A,
PENK, PITX2, PROM1, PRRX1, RARRES1, RASD1, RELN, RGMA, RGS1, RPS4Y2, SFRP2, SLITRK6, SMOC1,
SMOC2, SOX11, STMN2, TAC1, RSPO3, TNFSF7, TNNT2, TRH, TUBB4, UGT2B7, WISP2, ZIC1 and ZIC2.
The cell line SM42 is positive for the markers: COL15A1, EGR2, FST, GDF5, TMEM119, MMP1, MSX1, MSX2,
PRELP, PRRX1, PRRX2, SFRP2, SRCRB4D, ZIC1 and ZIC2 and is negative for the markers: ACTC, AGC1, AKR1C1,
ALDH1A1, ANXA8, APCDD1, AQP1, ATP8B4, CFB, BMP4, C3, C6, C7, C20orf103, CCDC3, CD24, CDH3,
CLDN11, CNTNAP2, CRIP1, CRYAB, DIO2, METTL7A, DKK2, DLK1, DPT, EMID1, FGFR3, TMEM100, FOXF2,
GABRB1, GAP43, GJB2, GSC, HOXA5, HSD11B2, HSPA6, ID4, IFI27, KIAA0644, KRT14, KRT17, KRT19, KRT34,
IGFL3, LOC92196, MFAP5, MEOX1, MEOX2, MGP, MX1, MYBPH, MYH3, MYH11, NLGN4X, NPPB, OGN,
OLR1, PAX2, PAX9, PDE1A, PENK, PITX2, PRG4, PROM1, RARRES1, RASD1, RELN, RGS1, SLITRK6, SMOC1,
SMOC2, SNAP25, STMN2, TAC1, RSPO3, TNFSF7, TNNT2, TRH, TUBB4 and UGT2B7.
The cell line SM44 is positive for the markers: CDH6, COMP, CRLF1, CRYAB, EGR2, FOXF1, FST, GDF5, HTRA3,
MGP, MMP1, MSX2, POSTN, PRELP, PRRX2, SYT12 and TSLP and is negative for the markers: ACTC, AGC1,
AKR1C1, ALDH1A1, ANXA8, APCDD1, AQP1, AREG, ATP8B4, CFB, BMP4, C3, C6, C7, C20orf103, CD24, CDH3,
CLDN11, CNTNAP2, COL15A1, COL21A1, COP1, CXADR, METTL7A, DKK2, DLK1, DPT, EMID1, FGFR3,
TMEM100, FMO1, FMO3, FOXF2, GABRB1, GDF10, GJB2, GSC, HOXA5, HSD11B2, HSD17B2, HSPA6, ID4,
IFI27, IFIT3, IGF2, KRT14, KRT17, KRT19, KRT34, IGFL3, LOC92196, MFAP5, MEOX1, MEOX2, MX1, MYBPH,
MYH3, MYH11, MYL4, NLGN4X, NPPB, OGN, OLR1, OSR2, PAX2, PAX9, PDE1A, PENK, PRG4, PROM1, PTN,
PTPRN, RARRES1, RASD1, RELN, RGS1, SFRP2, SLITRK6, SMOC1, SMOC2, SNAP25, STMN2, TAC1, RSPO3,
TNFSF7, TNNT2, TRH, TUBB4, UGT2B7, WISP2, ZD52F10, ZIC1 and ZIC2.
The cell line SM49 is positive for the markers: FOXF1, FOXF2, FST, GAP43, GDF5, HSPB3, HTRA3, IGFBP5, MGP,
MMP1, MSX2, POSTN, PRELP, PRRX2, PTN, RGMA, SOD3, SRCRB4D and SYT12 and is negative for the markers:
ACTC, AGC1, AKR1C1, ALDH1A1, ANXA8, APCDD1, AQP1, AREG, CFB, BMP4, C6, C7, C20orf103, CD24,
CDH3, CLDN11, CNTNAP2, COL15A1, COL21A1, DIO2, METTL7A, DPT, EMID1, FGFR3, TMEM100, FMO1,
GABRB1, GDF10, GJB2, GSC, HOXA5, HSD11B2, HSD17B2, HSPA6, ID4, IFI27, IFIT3, KIAA0644, KRT14,
KRT17, KRT19, KRT34, LAMC2, IGFL3, LOC92196, MFAP5, MEOX1, MEOX2, MYBPH, MYH3, MYH11, MYL4,
NLGN4X, TAGLN3, NPAS1, NPPB, OGN, OLR1, OSR2, PAX2, PAX9, PDE1A, PENK, PITX2, PRG4, PROM1,
RARRES1, RELN, RGS1, SMOC1, SMOC2, SNAP25, TAC1, RSPO3, THY1, TNFSF7, TNNT2, TRH, TUBB4,
UGT2B7, WISP2, ZIC1 and ZIC2
The cell line SM8 is positive for the markers: BEX1, CDH6, FOXF1, FST, GDF5, GDF10, IGF2, IGFBP5, MMP1,
MSX1, TFPI2, TSLP and ZIC2 and is negative for the markers: ACTC, AGC1, AKR1C1, ALDH1A1, ANXA8,
APCDD1, AQP1, ATP8B4, CFB, BMP4, C3, C6, C7, PRSS35, C20orf103, CCDC3, CDH3, CLDN11, COL21A1,
COMP, CRYAB, DIO2, METTL7A, DKK2, DLK1, DPT, EMID1, FGFR3, TMEM100, FMO1, FMO3, FOXF2,
GABRB1, GJB2, GSC, HOXA5, HSD11B2, HSD17B2, HSPA6, HSPB3, ICAM5, ID4, IFI27, KCNMB1, KIAA0644,
KRT14, KRT17, KRT34, TMEM119, IGFL3, LOC92196, MFAP5, MASP1, MEOX1, MEOX2, MGP, MX1, MYBPH,
MYH3, MYH11, MYL4, NLGN4X, NPAS1, NPPB, OGN, OLR1, OSR2, PAX2, PAX9, PDE1A, PENK, PITX2,
POSTN, PRELP, PRG4, PROM1, PRRX1, PTGS2, RGMA, RGS1, S100A4, SFRP2, SLITRK6, SMOC2, STMN2,
TAC1, RSPO3, TNFSF7, TNNT2, TRH, TUBB4, UGT2B7, WISP2 and ZD52F10.
The cell line T14 is positive for the markers: BEX1, PRSS35, CCDC3, COL15A1, CRIP1, CRYAB, FST, HTRA3, IGF2,
KCNMB1, KRT17, KRT19, LAMC2, PITX2, POSTN, S100A4, SOX11, THY1 and TNNT2 and is negative for the
markers: AGC1, ALDH1A1, AQP1, AREG, ATP8B4, CFB, C3, C6, C7, C20orf103, CDH3, CLDN11, CNTNAP2,
COP1, CXADR, METTLT7A, DLK1, DPT, EGR2, EMID1, TMEM100, FMO1, FMO3, FOXF1, FOXF2, GABRB1,
GDF10, GJB2, GSC, HOXA5, HSD11B2, HSD17B2, HSPA6, HSPB3, IFI27, IGFBP5, KIAA0644, KRT14, IGFL3,
LOC92196, MASP1, MEOX1, MEOX2, MGP, MX1, MYH3, IL32, NLGN4X, TAGLN3, NPPB, OGN, OLR1, OSR2,
PAX2, PAX9, PDE1A, PENK, PRG4, PROM1, PTGS2, PTPRN, RARRES1, RASD1, RELN, RGS1, SLITRK6,
SMOC1, SMOC2, SNAP25, SOD3, STMN2, TAC1, TFPI2, RSPO3, TNFSF7, TRH, TUBB4, WISP2, ZD52F10, ZIC1
and ZIC2.
The group of cell lines T4 and T23 are positive for the markers: BEX1, CCDC3, DKK2, KRT19 and LAMC2 and are
negative for the markers: ALDH1A1, APCDD1, AQP1, CFB, C3, C6, C20orf103, CDH3, CLDN11, CNTNAP2,
COL15A1, COMP, CRLF1, METTL7A, DPT, EMID1, TMEM100, FMO3, FOXF2, GDF10, GJB2, GSC, HOXA5,
HSD11B2, HSPA6, IFI27, IL1R1, KRT14, IGFL3, LOC92196, MASP1, MEOX1, MEOX2, MGP, MX1, MYBPH,
MYH3, MYH11, NLGN4X, NPAS1, OGN, OLR1, PAX2, PAX9, PDE1A, PENK, PROM1, PRRX2, PTPRN,
RARRES1, RASD1, RGMA, RGS1, RPS4Y2, SFRP2, SLITRK6, SMOC1, SMOC2, SNAP25, STMN2, SYT12, TAC1,
RSPO3, TNFSF7, TRH, WISP2, ZD52F10 and ZIC1.
The group of cell lines T36 and T42 are positive for the markers: BEX1, CCDC3, CDH6, CRIP1, FST, HTRA3, KRT17,
PTN, S100A4, SRCRB4D, THY1 and ZIC2 and are negative for the markers: AGC1, ALDH1A1, APCDD1, AREG,
ATP8B4, C3, C6, C7, PRSS35, C20orf103, CDH3, CLDN11, CNTNAP2, CRLF1, METTL7A, DLK1, DPT, EMID1,
FMO1, FMO3, FOXF2, GJB2, GSC, HOXA5, HSD11B2, HSD17B2, HSPA6, HSPB3, IFI27, KRT14, IGFL3,
LOC92196, MFAP5, MASP1, MEOX1, MEOX2, MGP, MMP1, MYBPH, MYH3, NLGN4X, TAGLN3, NPAS1, NPPB,
OGN, OLR1, PAX9, PDE1A, PENK, PRG4, PROM1, PTPRN, RARRES1, RASD1, RELN, RGS1, SLITRK6, SMOC2,
SNAP25, STMN2, TAC1, RSPO3, TRH, TUBB4 and WISP2.
The group of cell lines T43 and T44 are positive for the markers: BEX1, PRSS35, CCDC3, CDH6, COL21A1, CRIP1,
CRYAB, ICAM5, KRT17, LAMC2, POSTN, S100A4, SFRP2 and THY1 and are negative for the markers:
AGC1, ALDH1A1, APCDD1, AQP1, AREG, ATP8B4, C3, C6, C7, C20orf103, CDH3, CNTNAP2, COP1, METTL7A,
DLK1, DPT, EMID1, FMO1, FMO3, FOXF1, FOXF2, GABRB1, GDF10, GJB2, GSC, HOXA5, HSD11B2, HSD17B2,
HSPA6, IFI27, IGFBP5, IGFL3, LOC92196, MEOX1, MEOX2, MGP, NLGN4X, TAGLN3, NPPB, OGN, OLR1,
OSR2, PAX2, PAX9, PDE1A, PRG4, PROM1, RARRES1, RASD1, RELN, RGS1, SLITRK6, SMOC1, SMOC2,
SNAP25, STMN2, TAC1, TRH, TUBB4, UGT2B7, WISP2, ZD52F10 and ZIC2.
The cell line U18 is positive for the markers: ANXA8, BEX1, PRSS35, CCDC3, CDH6, CRYAB, DKK2, KRT19,
MYH11, NPPB, TNNT2 and ZIC2 and is negative for the markers: ACTC, AGC1, ALDH1A1, APCDD1, AQP1, AREG,
ATP8B4, CFB, C3, C6, C7, C20orf103, CD24, CDH3, CLDN11, CNTNAP2, COL15A1, COP1, CRLF1, DIO2,
METTL7A, DPT, EGR2, EMID1, TMEM100, FMO1, FMO3, FOXF1, FOXF2, GABRB1, GDF10, GJB2, GSC,
HOXA5, HSD11B2, HSD17B2, HSPA6, HSPB3, IFI27, IGF2, IGFBP5, KIAA0644, KRT14, TMEM119, IGFL3,
LOC92196, MEOX1, MEOX2, MGP, MX1, MYBPH, MYH3, NLGN4X, OGN, OLR1, PAX2, PAX9, PDE1A, PENK,
PROM1, PTPRN, RARRES1, RASD1, RELN, RGS1, SFRP2, SLITRK6, SMOC1, SMOC2, SNAP25, SOD3, STMN2,
TAC1, TFPI2, RSPO3, THY1, TNFSF7, TRH, TUBB4, WISP2 and ZIC1.
The group of cell lines U30, U30 and U31 are positive for the markers: BEX1, CDH6, CRYAB, KCNMB1, KRT17,
MYH11, ZIC1 and ZIC2 and are negative for the markers: ALDH1A1, ATP8B4, C3, C7, C20orf103, CD24, CDH3,
CLDN11, CNTNAP2, COP1, CRLF1, METTL7A, DPT, FMO1, FMO3, FOXF1, FOXF2, GABRB1, GSC, HOXA5,
HSD11B2, HSD17B2, HSPA6, IFI27, KIAA0644, KRT14, MEOX2, MGP, MYH3, OGN, OLR1, PAX2, PAX9,
PDE1A, PROM1, PTPRN, RASD1, RGS1, SFRP2, SMOC1, SNAP25, TAC1, TNNT2, TRH, TUBB4 and WISP2.
The cell line W11 is positive for the markers: COL15A1, COL21A1, DIO2, DLK1, FMO1, FOXF1, FOXF2, FST,
HTRA3, IGF2, IL1R1, TMEM119, OGN, PRRX2, PTN, SERPINA3, SLITRK6, SOD3, TFPI2 and WISP2 and is
negative for the markers: ACTC, AGC1, AKR1C1, ALDH1A1, ANXA8, APCDD1, AQP1, ATP8B4, CFB, C3, C6, C7,
C20orf103, CCDC3, CD24, CDH3, CLDN11, CNTNAP2, CRIP1, CRYAB, CXADR, DKK2, EMID1, FGFR3, GAP43,
GDF10, GJB2, GSC, HOXA5, HSD11B2, HSD17B2, HSPA6, HSPB3, ID4, IFI27, INA, KRT14, KRT17, KRT19,
KRT34, LAMC2, IGFL3, LOC92196, MFAP5, MEOX1, MEOX2, MGP, MMP1, MX1, MYBPH, MYH3, MYH11,
MYL4, IL32, NLGN4X, NPAS1, NPPB, OLR1, PAX2, PAX9, PENK, PITX2, POSTN, PRG4, PROM1, RASD1,
RELN, RGS1, SMOC1, SMOC2, STMN2, TAC1, RSPO3, THY1, TNFSF7, TNNT2, TRH, TUBB4, UGT2B7,
ZD52F10, ZIC1 and ZIC2.
The cell line W2 is positive for the markers: BEX1, CD24, COL21A1, FST, HTRA3, ICAM5, IGF2, IGFBP5, IL1R1,
KRT19, LAMC2, TMEM119, MSX1, MSX2, PTN, SERPINAB, SFRP2, SOD3, SOX11, SRCRB4D and ZIC2 and is
negative for the markers: AGC1, AKR1C1, ALDH1A1, APCDD1, ATP8B4, BMP4, C6, C7, C20orf103, CCDC3,
CDH3, CLDN11, CNTNAP2, COL15A1, COMP, COP1, CRLF1, DKK2, DLK1, DPT, EGR2, EMID1, TMEM100,
FMO3, FOXF2, GAP43, GDF10, GSC, HOXA5, HSD11B2, HSPA6, ID4, IFI27, INA, KCNMB1, KIAA0644, KRT14,
KRT17, IGFL3, LOC92196, MEOX1, MEOX2, MGP, MYBPH, MYH3, MYH11, NLGN4X, NPPB, OGN, OLR1,
OSR2, PAX2, PAX9, PDE1A, PENK, PITX2, PRG4, PROM1, PTGS2, RARRES1, RASD1, RELN, RGMA, RGS1,
SLITRK6, SMOC1, SMOC2, STMN2, SYT12, TAC1, TNFSF7, TNNT2, TRH, TSLP, TUBB4 and ZIC1.
The cell line W3 is positive for the markers: BEX1, CRIP1, FOXF1, FST, GDF5, HSPA6, HTRA3, IGF2, IGFBP5,
KRT19, LAMC2, MMP1, MSX1, POSTN, PTPRN and TFPI2 and is negative for the markers: ACTC, AGC1,
ALDH1A1, ANXA8, APCDD1, AQP1, ATP8B4, CFB, BMP4, C6, C7, PRSS35, C20orf103, CCDC3, CDH3, CLDN11,
CNTNAP2, COL15A1, COL21A1, COMP, DIO2, METTL7A, DKK2, DLK1, DPT, EGR2, EMID1, FGFR3, FMO1,
FMO3, FOXF2, GAP43, GDF10, GJB2, GSC, HOXA5, HSD11B2, HSD17B2, IFI27, IFIT3, INA, KIAA0644, KRT14,
KRT17, IGFL3, LOC92196, MEOX1, MEOX2, MGP, MX1, MYBPH, MYH3, MYH11, MYL4, IL32, NLGN4X,
NPPB, OGN, OSR2, PAX2, PAX9, PDE1A, PENK, PRELP, PRG4, PROM1, PRRX1, RARRES1, RELN, RGMA,
RGS1, SLITRK6, SMOC1, SMOC2, SOX11, SYT12, TAC1, RSPO3, THY1, TNFSF7, TNNT2, TRH, TUBB4,
UGT2B7, ZIC1 and ZIC2.
The cell line W8 is positive for the markers: AQP1, CDH6, DIO2, DLK1, EMID1, FOXF1, FOXF2, FST, HTRA3,
IL1R1, MSX1, MSX2, PRRX2, PTN, SLITRK6, SRCRB4D, TSLP and ZIC2 and is negative for the markers: ACTC,
AGC1, AKR1C1, ALDH1A1, ANXA8, APCDD1, BMP4, C6, C7, C20orf103, CCDC3, CD24, CDH3, CLDN11,
CNTNAP2, CRLF1, CRYAB, CXADR, DKK2, DPT, EGR2, FGFR3, TMEM100, GABRB1, GAP43, GDF10, GJB2,
GSC, HOXA5, HSD11B2, HSD17B2, HSPA6, HSPB3, ID4, IFI27, IFIT3, INA, KCNMB1, KRT14, KRT17, KRT34,
IGFL3, LOC92196, MFAP5, MEOX1, MEOX2, MX1, MYBPH, MYH3, MYH11, MYL4, NLGN4X, NPPB, OLR1,
PAX2, PAX9, PENK, PITX2, POSTN, PRELP, PROM1, PRRX1, RARRES1, RASD1, RGMA, RGS1, SMOC1,
SMOC2, STMN2, SYTE2, TAC1, RSPO3, THY1, TNFSF7, TNNT2, TRH, TUBB4, UGT2B7, WISP2, ZD52F10 and
ZIC1.
The cell line X4 is positive for the markers: ACTC, AQP1, BEX1, BMP4, CD24, CDH6, CLDN11
CRYAB, CXADR, HTRA3, INA, KRT17, KRT19, LAMC2, MMP1, IL32, NLGN4X, TAGLN3, NPPB, PAX2,
PROM1, RASD1, RELN and UGT2137 and is negative for the markers: AGC1, ALDH1A1, APCDD1, ATP8B4, CFB,
C3, C6, C7, C20orf103, CCDC3, CDH3, CNTNAP2, COL15A1, COL21A1, COMP, COP1, CRLF1, DIO2, METTL7A,
DKK2, DLK1, DPT, EGR2, EMID1, TMEM100, FMO1, FMO3, FOXF1, FOXF2, FST, GABRB1, GAP43, GDF10,
GJB2, GSC, HOXA5, HSD11B2, HSD17B2, HSPA6, ID4, IFI27, IFIT3, IGF2, IL1R1, KCNMB1, KIAA0644,
TMEM119, IGFL3, LOC92196, MFAP5, MASP1, MEOX1, MEOX2, MGP, MX1, MYBPH, MYH3, MYL4, OGN,
OSR2, PAX9, PDE1A, PENK, PITX2, PRELP, PRRX1, PRRX2, PTGS2, PTN, RARRES1, RGMA, RGS1, SERPINA3,
SLITRK6, SMOC1, SMOC2, SOD3, TAC1, RSPO3, TNNT2, TRH, TUBB4, WISP2, ZD52F10, ZIC1 and ZIC2.
The cell line X5.4 is positive for the markers: ACTC, CD24, CLDN11, CRIP1, CRYAB, HTRA3, KRT19, KRT34,
LAMC2, MMP1, IL32, NLGN4X, TAGLN3, NPPB, PAX2, POSTN, RELN, S100A4, SFRP2, SRCRB4D, THY1 and
UGT2B7 and is negative for the markers: AGC1, ALDH1A1, APCDD1, AREG, ATP8B4, CFB, C3, C6, C7, C20orf103,
CNTNAP2, COL21A1, COMP, COP1, CRLF1, DIO2, METTLTA, DKK2, DLK1, DPT, EMID1, TMEM100, FMO1,
FMO3, FOXF1, FOXF2, GABRB1, GAP43, GDF10, GJB2, GSC, HOXA5, HSD11B2, HSD17B2, HSPA6, ID4, IFI27,
IFIT3, IGF2, KIAA0644, TMEM119, IGFL3, MASP1, MEOX2, MSX1, MX1, MYBPH, MYH3, MYL4, NPAS1, OGN,
OSR2, PAX9, PDE1A, PENK, PRELP, PRRX1, PRRX2, PTPRN, RARRES1, RGMA, RGS1, SLITRK6, SMOC1,
SMOC2, SNAP25, SOD3, TAC1, RSPO3, TNNT2, TRH, TUBB4, WISP2, ZD52F10, ZIC1 and ZIC2.
The cell line X5 is positive for the markers: ACTC, AKR1C1, BEX1, CLDN11, COMP, CRIP1, CRYAB, GDF5,
HTRA3, KIAA0644, KRT14, KRT19, KRT34, LAMC2, MFAP5, MEOX2, MGP, MMP1, PENK, PITX2, POSTN,
PTGS2, S100A4 and THY1 and is negative for the markers: AGC1, ALDH1A1, ANXA8, APCDD1, AQP1, AREG,
ATP8B4, C6, C7, C20orf103, CCDC3, CDH6, CNTNAP2, COL15A1, COL21A1, COP1, CXADR, DIO2, DKK2,
DLK1, DPT, EMID1, FGFR3, TMEM100, FMO1, FMO3, FOXF1, FOXF2, GAP43, GDF10, HSD11B2, HSD17B2,
HSPA6, IFI27, IFIT3, IGF2, IGFL3, LOC92196, MEOX1, MSX1, MSX2, MYBPH, MYH3, MYH11, MYL4, NLGN4X,
NPPB, OGN, OLR1, PAX2, PAX9, PDE1A, PROM1, PTPRN, RASD1, RELN, RGS1, SERPINA3, SFRP2, SMOC2,
SNAP25, STMN2, SYT12, TAC1, RSPO3, TNNT2, TRH, TUBB4, UGT2B7, WISP2, ZD52F10, ZIC1 and ZIC2.
The group of cell lines X7PEND12 and X7PEND24 are positive for the markers: AQP1, BEX1, CDH3, DIO2, DLK1,
FOXF1, FST, GABRB1, IGF2, IGFBP5, IL1R1, KIAA0644, MSX1, PODN, PRRX2, SERPINA3, SOX11, SRCRB4D
and TFPI2 and are negative for the markers: ACTC, AGC1, AKR1C1, ALDH1A1, ANXA8, APCDD1, AREG, CFB, C3,
C6, C7, PRSS35, CCDC3, CD24, CLDN11, COMP, COP1, CXADR, DKK2, EMID1, FGFR3, FMO1, FMO3, GAP43,
GDF10, GSC, HOXA5, HSD11B2, HSPA6, HTRA3, ICAM5, ID4, IFI27, IFIT3, INA, KCNMB1, KRT14, KRT17,
KRT34, IGFL3, LOC92196, MFAP5, MASP1, MEOX1, MEOX2, MMP1, MX1, MYBPH, MYH3, MYH11, MYL4,
IL32, NLGN4X, NPPB, OGN, OSR2, PAX2, PAX9, PENK, PITX2, PRELP, PRG4, PRRX1, RARRES1, RELN,
RGMA, SFRP2, SMOC1, SMOC2, SOD3, SYT12, TAC1, TNFSF7, TRH, TSLP, TUBB4, UGT2B7, WISP2, ZD52F10,
ZIC1 and ZIC2.
The group of cell lines X7PEND9 and X7PEND16 are positive for the markers: BEX1, CDH6, DLK1, TMEM100,
FOXF1, FOXF2, IGF2, IGFBP5, IL1R1, KIAA0644, TMEM119, MGP, MSX1, MSX2, PDE1A, PODN, PRRX2, PTN,
S100A4, SERPINA3, SNAP25, SOX11 and SRCRB4D and are negative for the markers: ACTC, AGC1, AKR1C1,
ALDH1A1, ANXA8, AREG, ATP8B4, BMP4, C3, C20orf103, CCDC3, CD24, CDH3, CNTNAP2, COP1, CRYAB,
CXADR, METTL7A, DKK2, EMID1, FGFR3, FMO1, GDF10, GSC, HOXA5, HSD11B2, HSD17B2, HSPA6, HSPB3,
ICAM5, ID4, IFI27, INA, KCNMB1, KRT14, KRT17, KRT34, IGFL3, LOC92196, MFAP5, MASP1, MEOX1,
MEOX2, MMP1, MYBPH, MYH3, MYH11, MYL4, IL32, NLGN4X, NPAS1, NPPB, OLR1, OSR2, PAX2, PAX9,
PENK, PITX2, PRELP, PRG4, PROM1, PTPRN, RASD1, RELN, RGS1, SFRP2, SMOC1, SMOC2, SOD3, SYT12,
TAC1, RSPO3, THY1, TNFSF7, TNNT2, TRH, TSLP, TUBB4, UGT2B7, ZD52F10, ZIC1 and ZIC2.
The cell line X7PEND30 is positive for the markers: BEX1, PRSS35, CDH6, COL15A1, DIO2, DLK1, DPT,
TMEM100, FMO1, FMO3, FOXF1, FOXF2, FST, HSPB3, IGF2, IGFBP5, IL1R1, KIAA0644, KRT19, LAMC2,
TMEM119, MGP, MSX1, PDE1A, PODN, PRRX2, S100A4, SERPINA3, SOX11 and SRCRB4D and is negative for the
markers: ACTC, AGC1, AKR1C1, ALDH1A1, ANXA8, APCDD1, AQP1, AREG, ATP8B4, C3, C7, C20orf103,
CCDC3, CD24, CDH3, CLDN11, CNTNAP2, COP1, CXADR, DKK2, EMID1, FGFR3, GAP43, GDF5, GDF10, GJB2,
GSC, HOXA5, HSD11B2, HSD17B2, HSPA6, HTRA3, ICAM5, ID4, IFI27, INA, KCNMB1, KRT14, KRT17, KRT34,
IGFL3, LOC92196, MFAP5, MASP1, MEOX1, MEOX2, MMP1, MYBPH, MYH3, MYH11, MYL4, IL32, NLGN4X,
NPPB, OSR2, PAX2, PAX9, PENK, PITX2, PRELP, PRRX1, PTGS2, PTPRN, RELN, RGS1, SFRP2, SMOC1,
SMOC2, SOD3, STMN2, SYT12, TAC1, RSPO3, THY1, TNFSF7, TNNT2, TRH, TSLP, TUBB4, UGT2B7, WISP2,
ZD52F10, ZIC1 and ZIC2.
The cell line X7SKEL2 is positive for the markers: APCDD1, BEX1, C6, C7, PRSS35, COL21A1, CRIP1, CRLF1,
CRYAB, DLK1, TMEM100, FMO1, FOXF2, GDF5, HSD11B2, IGF2, IGFBP5, KRT19, LAMC2, TMEM119, MGP,
NPAS1, PRRX2, PTPRN, RGMA, S100A4, SERPINA3, SNAP25 and SOX11 and is negative for the markers: ACTC,
AGC1, AKR1C1, ALDH1A1, ANXA8, AQP1, AREG, ATP8B4, CFB, BMP4, C3, C20orf103, CCDC3, CD24, CDH3,
CDH6, CLDN11, CNTNAP2, COMP, COP1, CXADR, DIO2, METTL7A, DKK2, DPT, EGR2, EMID1, FGFR3,
FOXF1, GABRB1, GDF10, GJB2, GSC, HOXA5, HSD17B2, HSPA6, HTRA3, ID4, IFI27, IFIT3, KCNMB1,
KIAA0644, KRT14, KRT17, KRT34, IGFL3, LOC92196, MFAP5, MASP1, MEOX1, MEOX2, MMP1, MSX2, MX1,
MYBPH, MYH3, MYH11, MYL4, IL32, NLGN4X, NPPB, OGN, OLR1, OSR2, PAX2, PAX9, PENK, PITX2, POSTN,
PRELP, PROM1, PRRX1, PTGS2, PTN, RARRES1, RELN, RGS1, SLITRK6, SMOC1, SMOC2, SOD3, STMN2,
SYT12, TAC1, TFPI2, RSPO3, THY1, TNFSF7, TRH, TSLP, TUBB4, UGT2B7, ZIC1 and ZIC2.
The cell line X7SKEL22 is positive for the markers: ACTC, BEX1, C7, PRSS35, COL21A1, CRIP1, CRYAB, DIO2,
DPT, EGR2, FMO3, FOXF1, FOXF2, FST, GJB2, HSPB3, IGF2, IGFBP5, IL1R1, KRT19, LAMC2, TMEM119, MGP,
NPAS1, PODN, PRRX2, SERPINA3, SOX11 and SRCRB4D and is negative for the markers: AGC1, AKR1C1,
ALDH1A1, ANXA8, AQP1, AREG, ATP8B4, CFB, BMP4, C3, C20orf103, CCDC3, CD24, CDH3, CDH6, CLDN11,
CNTNAP2, COL15A1, COMP, COP1, CXADR, METTL7A, DKK2, DLK1, EMID1, FGFR3, TMEM100, GABRB1,
GAP43, GDF5, GDF10, GSC, HOXA5, HSD17B2, HSPA6, HTRA3, ICAM5, ID4, IFI27, IFIT3, KCNMB1, KRT14,
KRT17, KRT34, IGFL3, LOC92196, MFAP5, MASP1, MEOX1, MEOX2, MMP1, MSX1, MSX2, MX1, MYBPH,
MYH3, MYH11, IL32, NLGN4X, NPPB, OGN, OLR1, OSR2, PAX2, PAX9, PENK, PITX2, POSTN, PRELP, PRG4,
PROM1, PRRX1, PTN, RARRES1, RASD1, RELN, RGS1, SFRP2, SLITRK6, SMOC1, SMOC2, SOD3, STMN2,
SYT12, TAC1, TFPI2, RSPO3, TNFSF7, TNNT2, TRH, TSLP, TUBB4, UGT2B7, ZD52F10, ZIC1 and ZIC2.
The group of cell lines X7SKEL4, X7SKEL6 and X7SKEL7 are positive for the markers: BEX1, COL21A1, CRLF1,
DLK1, FMO1, FMO3, FOXF1, FOXF2, HSD11B2, IGF2, IGFBP5, IL1R1, TMEM119, PRRX2, RGMA, SERPINA3,
SNAP25, SOX11 and SRCRB4D and are negative for the markers: ACTC, AGC1, AKR1C1, ALDH1A1, ANXA8,
AQP1, AREG, ATP8B4, CFB, BMP4, C3, C20orf103, CCDC3, CD24, CDH3, CLDN11, CNTNAP2, COL15A1,
COMP, COP1, CXADR, DKK2, EMID1, FGFR3, GDF10, GJB2, GSC, HOXA5, HSD17B2, HSPA6, HTRA3, ID4,
IFI27, IFIT3, INA, KCNMB1, KRT14, KRT17, KRT34, IGFL3, LOC92196, MFAP5, MASP1, MEOX1, MEOX2,
MMP1, MYBPH, MYH3, MYH11, MYL4, IL32, NLGN4X, NPPB, OLR1, OSR2, PAX2, PENK, PITX2, POSTN,
PRELP, PROM1, RELN, RGS1, SFRP2, SLITRK6, SMOC1, SMOC2, SOD3, STMN2, SYT12, TAC1, TFPI2, RSPO3,
THY1, TNFSF7, TNNT2, TRH, TSLP, TUBB4 and ZIC1.
The cell line X7SMOO12 is positive for the markers: BEX1, CDH6, COL21A1, CRIP1, DIO2, DLK1, EGR2, FOXF1,
FOXF2, FST, IGF2, IGFBP5, TMEM119, MSX1, MSX2, MX1, PODN, POSTN, PRRX2, PTN, S100A4, SERPINA3,
SOX11, TFPI2, WISP2 and ZIC2 and is negative for the markers: ACTC, AGC1, AKR1C1, ALDH1A1, ANXA8,
APCDD1, AQP1, AREG, CFB, C3, C6, C7, C20orf103, CCDC3, CD24, CLDN11, CNTNAP2, COMP, COP1, CRYAB,
CXADR, METTL7A, DKK2, EMID1, FGFR3, TMEM100, GABRB1, GAP43, GDF10, GJB2, GSC, HOXA5,
HSD11B2, HSD17B2, HSPA6, HSPB3, HTRA3, ICAM5, ID4, IFI27, IL1R1, KCNMB1, KRT14, KRT17, KRT34,
IGFL3, LOC92196, MFAP5, MASP1, MEOX1, MEOX2, MGP, MMP1, MYBPH, MYH3, MYH11, MYL4, IL32,
NLGN4X, NPPB, OGN, OSR2, PAX2, PAX9, PDE1A, PENK, PITX2, PRELP, PRG4, PTGS2, RARRES1, RGS1,
SFRP2, SMOC1, SMOC2, SOD3, SYT12, TAC1, RSPO3, TNFSF7, TRH, TSLP, TUBB4, UGT2B7, ZD52F10 and
ZIC1.
The cell line X7SMOO19 is positive for the markers: BEX1, CDH6, COL15A1, COL21A1, COMP, CRIP1, DLK1,
EGR2, FMO1, FMO3, FOXF1, FOXF2, FST, HSPA6, IGF2, IGFBP5, KIAA0644, KRT19, LAMC2, TMEM119,
MSX1, MSX2, OGN, PODN, PRRX2, RGMA, S100A4, SERPINA3, SNAP25, SOX11, SRCRB4D, TNNT2 and ZIC2
and is negative for the markers: ACTC, AGC1, AKR1C1, ALDH1A1, ANXA8, APCDD1, AREG, ATP8B4, C3, C6, C7,
C20orf103, CCDC3, CD24, CLDN11, COP1, CXADR, DIO2, METTL7A, DKK2, DPT, EMID1, TMEM100, GABRB1,
GJB2, GSC, HOXA5, HSD11B2, HSD17B2, HTRA3, ICAM5, ID4, IFI27, IL1R1, INA, KCNMB1, KRT14, KRT17,
KRT34, IGFL3, LOC92196, MFAP5, MASP1, MEOX1, MEOX2, MMP1, MYBPH, MYH3, MYH11, MYL4, IL32,
NLGN4X, NPPB, OLR1, OSR2, PAX2, PAX9, PENK, PITX2, PRG4, PROM1, PTPRN, RARRES1, RELN, RGS1,
SFRP2, SLITRK6, SMOC1, SMOC2, SOD3, STMN2, SYT12, TAC1, RSPO3, TNFSF7, TRH, TSLP, TUBB4,
UGT2B7, WISP2, ZD52F10 and ZIC1.
The cell line X7SMOO25 is positive for the markers: AQP1, ATP8B4, BEX1, CDH3, COL21A1, CRIP1, DLK1,
FOXF1, FOXF2, FST, GABRB1, HSPB3, IGF2, IGFBP5, IL1R1, KRT19, LAMC2, TMEM119, MSX1, MSX2, PODN,
POSTN, PRRX2, PTN, RGMA, S100A4, SERPINA3, SLITRK6, SOX11, SRCRB4D, TFPI2, RSPO3 and THY1 and is
negative for the markers: ACTC, AGC1, AKR1C1, ANXA8, APCDD1, AREG, CFB, BMP4, C3, C6, C7, PRSS35,
C20orf103, CCDC3, CLDN11, COL15A1, COP1, CXADR, METTL7A, DKK2, EGR2, EMID1, FGFR3, TMEM100,
FMO1, FMO3, GDF10, GJB2, GSC, HOXA5, HSD11B2, HSD17B2, HSPA6, HTRA3, ICAM5, ID4, IFI27, INA,
KCNMB1, KRT14, KRT17, KRT34, IGFL3, LOC92196, MASP1, MEOX1, MEOX2, MGP, MYBPH, MYH3, MYH11,
MYL4, IL32, NLGN4X, NPPB, OGN, OLR1, OSR2, PAX2, PAX9, PDE1A, PENK, PITX2, PRELP, PRG4, PROM1,
PRRX1, PTPRN, RASD1, RELN, RGS1, SFRP2, SMOC1, SMOC2, SOD3, SYT12, TAC1, TNFSF7, TRH, TSLP,
TUBB4, UGT2B7, WISP2, ZD52F10, ZIC1 and ZIC2.
The cell line X7SMOO26 is positive for the markers: BEX1, CCDC3, CDH6, COL15A1, COL21A1, COMP, CRIP1,
CRLF1, CRYAB, DIO2, EGR2, FOXF1, FOXF2, FST, GDF10, HSPB3, IGF2, IGFBP5, KRT19, LAMC2, TMEM119,
MSX1, MSX2, NPAS1, PODN, POSTN, PRRX2, S100A4, SERPINA3, SOX11, SRCRB4D, TNNT2 and ZIC2 and is
negative for the markers: ACTC, AGC1, AKR1C1, ALDH1A1, ANXA8, APCDD1, AREG, ATP8B4, CFB, BMP4, C3,
C6, C7, C20orf103, CD24, CDH3, CLDN11, COP1, METTL7A, DLK1, DPT, EMID1, FGFR3, TMEM100, FMO1,
FMO3, GJB2, GSC, HOXA5, HSD11B2, HSPA6, HTRA3, ICAM5, ID4, IFI27, IL1R1, KCNMB1, KIAA0644, KRT14,
KRT34, IGFL3, LOC92196, MFAP5, MASP1, MEOX1, MEOX2, MGP, MMP1, MX1, MYBPH, MYH3, IL32,
NLGN4X, OGN, OLR1, OSR2, PAX2, PAX9, PDE1A, PENK, PITX2, PRELP, PRG4, PROM1, PTGS2, PTN, PTPRN,
RARRES1, RASD1, RELN, RGS1, SFRP2, SLITRK6, SMOC1, SMOC2, SNAP25, SOD3, STMN2, SYT12, TAC1,
TFPI2, RSPO3, THY1, TNFSF7, TRH, TSLP, TUBB4, UGT2B7, WISP2, ZD52F10 and ZIC1.
The group of cell lines X7SMOO9 and X7SMOO29 are positive for the markers; BEX1, COL21A1, CRIP1, CRLF1,
DIO2, DLK1, FOXF1, FOXF2, FST, IGF2, IGFBP5, KIAA0644, TMEM119, MSX1, PODN, POSTN, PRRX2, RGMA,
S100A4, SERPINA3, SNAP25, SOX11 and SRCRB4D and are negative for the markers: ACTC, AGC1, AKR1C1,
ALDH1A1, ANXA8, APCDD1, AQP1, AREG, ATP8B4, C3, C6, C7, PRSS35, C20orf103, CCDC3, CD24, CDH3,
CLDN11, COP1, CXADR, METTL7A, DKK2, EMID1, GDF10, GJB2, GSC, HOXA5, HSD11B2, HSD17B2, HSPA6,
HTRA3, ICAM5, ID4, IFI27, IL1R1, INA, KCNMB1, KRT14, KRT17, KRT19, KRT34, IGFL3, LOC92196, MFAP5,
MASP1, MEOX1, MEOX2, MYH3, MYH11, MYL4, IL32, NLGN4X, NPPB, OLR1, OSR2, PAX2, PAX9, PENK,
PITX2, PRELP, PROM1, PTPRN, RASD1, RELN, RGS1, SMOC1, SMOC2, SYT12, TAC1, TNFSF7, TRH, TSLP,
TUBB4, UGT2B7, ZD52F10 and ZIC1.
The cell line X7SMOO32 is positive for the markers: ACTC, BEX1, CDH6, COL21A1, CRIP1, CRLF1, DIO2, DLK1,
EGR2, FGFR3, FOXF1, FOXF2, FST, GABRB1, IGFBP5, KIAA0644, KRT19, LAMC2, TMEM119, MGP, MMP1,
MSX1, MSX2, PODN, POSTN, PRG4, PRRX2, PTN, RGMA, S100A4, SERPINA3, SOX11 and SRCRB4D and is
negative for the markers: AGC1, AKR1C1, ALDH1A1, ANXA8, APCDD1, AREG, ATP8B4, BMP4, C3, C6, C7,
PRSS35, C20orf103, CCDC3, CD24, CLDN11, CNTNAP2, COL15A1, COP1, CXADR, METTL7A, DKK2, DPT,
EMID1, TMEM100, FMO1, FMO3, GDF5, GDF10, GJB2, GSC, HOXA5, HSD11B2, HSD17B2, HSPA6, HSPB3,
HTRA3, ICAM5, ID4, IFI27, IL1R1, INA, KCNMB1, KRT14, KRT17, KRT34, IGFL3, LOC92196, MFAP5, MASP1,
MEOX1, MEOX2, MYBPH, MYH3, MYH11, MYL4, IL32, NLGN4X, NPPB, OGN, OLR1, OSR2, PAX2, PAX9,
PDE1A, PITX2, PRELP, PROM1, PTPRN, RASD1, RGS1, SFRP2, SMOC1, SMOC2, SOD3, STMN2, SYT12, TAC1,
RSPO3, TNFSF7, TNNT2, TRH, TSLP, TUBB4, UGT2B7, WISP2, ZD52F10, ZIC1 and ZIC2.
The cell line X7SMOO6 is positive for the markers: ACTC, BEX1, CNTNAP2, COL15A1, COL21A1, CRIP1, CRLF1,
CRYAB, DLK1, EGR2, FMO1, FMO3, FOXF1, FOXF2, FST, HSPB3, IGF2, IGFBP5, KRT19, LAMC2, TMEM119,
MGP, MSX1, MSX2, NPAS1, OGN, PODN, POSTN, PRRX2, RGMA, S100A4, SERPINA3, SNAP25, SOX11,
SRCRB4D, STMN2 and TNNT2 and is negative for the markers: AGC1, AKR1C1, ALDH1A1, ANXA8, APCDD1,
AQP1, AREG, ATP8B4, C3, C6, C7, C20orf103, CCDC3, CD24, CLDN11, COP1, CXADR, DIO2, METTL7A, DKK2,
EMID1, TMEM100, GAP43, GDF10, GJB2, GSC, HOXA5, HSD11B2, HSD17B2, HSPA6, HTRA3, ICAM5, ID4,
IFI27, IL1R1, INA, KCNMB1, KRT14, KRT17, KRT34, IGFL3, LOC92196, MFAP5, MASP1, MEOX1, MEOX2,
MYBPH, MYH3, MYH11, MYL4, IL32, NLGN4X, TAGLN3, NPPB, OSR2, PAX2, PAX9, PDE1A, PENK, PITX2,
PRG4, PRRX1, PTGS2, PTPRN, RASD1, RELN, RGS1, SFRP2, SMOC1, SMOC2, SYT12, TAC1, RSPO3, TNFSF7,
TRH, TSLP, TUBB4, UGT2B7, ZD52F10, ZIC1 and ZIC2.
The cell line X7SMOO7 is positive for the markers: ACTC, BEX1, CDH6, CRIP1, CRLF1, CRYAB, DLK1, EGR2,
FOXF1, FOXF2, FST, HSPA6, IGF2, IGFBP5, INA, LAMC2, MMP1, MSX1, MSX2, TAGLN3, POSTN, PRRX2,
PTGS2, PTPRN, RASD1, RELN, S100A4, SNAP25, SOX11, SRCRB4D, TAC1, TFPI2 and RSPO3 and is negative for
the markers: AGC1, AKR1C1, ALDH1A1, ANXA8, APCDD1, AQP1, AREG, CFB, BMP4, C3, C6, C7, C20orf103,
CCDC3, CDH3, CLDN11, CNTNAP2, COL15A1, COL21A1, COP1, CXADR, METTL7A, DKK2, DPT, EMID1,
FMO3, GAP43, GDF5, GDF10, GSC, HOXA5, HSD11B2, HSD17B2, HSPB3, HTRA3, ID4, IFI27, IFIT3, KCNMB1,
KIAA0644, KRT14, KRT17, IGFL3, LOC92196, MFAP5, MASP1, MEOX1, MEOX2, MGP, MYBPH, MYH3,
MYH11, MYL4, IL32, NLGN4X, NPPB, OGN, OLR1, OSR2, PAX2, PAX9, PDE1A, PENK, PITX2, PRELP, PRG4,
PROM1, PRRX1, PTN, RGMA, RGS1, SFRP2, SLITRK6, SMOC2, SOD3, STMN2, SYT12, TNNT2, TRH, TSLP,
TUBB4, WISP2 and ZIC1.
The group of cell lines Z1, Z6 and Z7 are positive for the markers: FST, GDF5, MMP1, MSX1, SRCRB4D, ZIC1 and
ZIC2 and are negative for the markers: ACTC, AGC1, AKR1C1, ALDH1A1, ANXA8, APCDD1, AQP1, AREG,
ATP8B4, CFB, BMP4, C3, C6, C7, C20orf103, CDH3, CLDN11, CNTNAP2, CRLF1, DIO2, METTL7A, DKK2,
DLK1, DPT, EMID1, FGFR3, TMEM100, FMO1, FMO3, FOXF2, GABRB1, GJB2, GSC, HOXA5, HSD11B2, HSPA6,
HSPB3, ID4, IFI27, IGF2, KCNMB1, KIAA0644, KRT14, IGFL3, LOC92196, MFAP5, MASP1, MEOX1, MEOX2,
MGP, MYBPH, MYH3, MYH11, NLGN4X, NPPB, OGN, OLR1, PAX2, PAX9, PDE1A, PENK, PITX2, PRG4,
PROM1, RARRES1, RASD1, RELN, RGS1, SFRP2, SMOC1, SMOC2, SNAP25, STMN2, SYT12, TAC1, RSPO3,
TNFSF7, TNNT2, TRH, TUBB4 and WISP2.
The group of cell lines Z11Rep1 and Z11Rep2 are positive for the markers: ATP8B4, CD24, DLK1, FOXF1, FST,
HTRA3, IGF2, IGFBP5, IL1R1, MSX1, NLGN4X, OSR2, PODN, PROM1, PRRX2, PTN, SOD3, SOX11, SRCRB4D,
STMN2 and TFPI2 and are negative for the markers: ACTC, AGC1, AKR1C1, ALDH1A1, ANXA8, APCDD1, AREG,
CFB, C6, C7, PRSS35, CCDC3, CDH3, CLDN11, CNTNAP2, COMP, CRIP1, CRLF1, DIO2, DKK2, DPT, EMID1,
FMO1, FMO3, GAP43, GDF10, GJB2, GSC, HOXA5, HSD11B2, HSPA6, HSPB3, IFI27, INA, KCNMB1, KIAA0644,
KRT14, KRT17, KRT34, LAMC2, IGFL3, LOC92196, MFAP5, MEOX1, MEOX2, MX1, MYBPH, MYH3, MYH11,
MYL4, IL32, NPPB, OLR1, PAX2, PITX2, RARRES1, RASD1, RGS1, SMOC1, SMOC2, SNAP25, TAC1, TNFSF7,
TNNT2, TRH, TUBB4, UGT2B7, WISP2, ZIC1 and ZIC2.
The cell line Z2 is positive for the markers: BEX1, CCDC3, EGR2, FOXF1, FOXF2, FST, GDF5, HSPB3, IGFBP5,
INA, TMEM119, MASP1, MMP1, MSX2, POSTN, PRELP, PRRX2, PTN, SRCRB4D, TFPI2 and TSLP and is negative
for the markers: ACTC, AGC1, AKR1C1, ALDH1A1, ANXA8, APCDD1, AQP1, AREG, CFB, BMP4, C3, C6, C7,
C20orf103, CD24, CDH3, CLDN11, CNTNAP2, COL21A1, DIO2, DKK2, DLK1, DPT, FGFR3, TMEM100, FMO1,
FMO3, GABRB1, GAP43, GDF10, GJB2, GSC, HOXA5, HSD11B2, HSD17B2, HSPA6, ID4, IFI27, KCNMB1,
KIAA0644, KRT14, KRT17, KRT34, IGFL3, LOC92196, MFAP5, MEOX1, MEOX2, MYBPH, MYH3, MYH11,
NLGN4X, NPPB, OGN, OSR2, PAX2, PAX9, PDE1A, PENK, PITX2, PRG4, PROM1, RARRES1, RASD1, RGS1,
SMOC1, SMOC2, SNAP25, STMN2, TAC1, RSPO3, TNFSF7, TNNT2, TRH, TUBB4, WISP2, ZIC1 and ZIC2.
The cell line MEL2 is positive for the markers: AKR1C1, AQP1, COL21A1, CRYAB, CXADR, DIO2, METTL7A,
DKK2, DLK1, HSD17B2, HSPB3, MGP, MMP1, MSX2, PENK, PRRX1, PRRX2, S100A4, SERPINA3, SFRP2,
SNAP25, SOX11, TFPI2 and THY1 and is negative for the markers: ACTC, ALDH1A1, AREG, CFB, C3, C20orf103,
CD24, CDH3, CDH6, CNTNAP2, COL15A1, COMP, COP1, CRLF1, FGFR3, FMO1, FMO3, FOXF2, FST, GABRB1,
GAP43, GDF5, GDF10, GJB2, GSC, HOXA5, HSD11B2, HSPA6, ICAM5, KCNMB1, KRT14, KRT17, KRT19,
KRT34, MASP1, MEOX1, MEOX2, MYBPH, MYH3, MYH11, TAGLN3, NPAS1, NPPB, OLR1, PAX2, PDE1A,
PITX2, PRG4, PTN, PTPRN, RASD1, RELN, RGS1, SMOC1, STMN2, TAC1, TNFSF7, TRH, TUBB4, WISP2, ZIC1
and ZIC2.
The cell line C4ELSR10 is positive for the markers: AKR1C1, ALDH1A1, ANXA8, AREG, CDH6, COP1, DIO2,
METTL7A, EGR2, FOXF1, HSD17B2, IGFBP5, KIAA0644, KRT19, KRT34, OLR1, PITX2, S100A4, STMN2 and
TFPI2 and is negative for the markers: ACTC, AQP1, C7, C20orf103, CD24, CDH3, CLDN11, CNTNAP2, COMP,
CRIP1, CRLF1, DKK2, DLK1, DPT, FGFR3, FMO1, GABRB1, GAP43, GDF10, GJB2, GSC, HSD11B2, HSPA6,
HSPB3, ICAM5, ID4, KRT14, KRT17, LAMC2, MPAP5, MASP1, MEOX1, MEOX2, MGP, MMP1, MSX1, MYBPH,
MYH3, MYH11, TAGLN3, NPAS1, NPPB, OGN, PAX2, PAX9, PENK, PRELP, PRG4, PRRX1, PRRX2, PTN, RELN,
RGS1, SERPINA3, SFRP2, SMOC1, SNAP25, SOX11, TAC1, TNNT2, TUBB4, WISP2, ZIC1 and ZIC2.
The cell line Z3 is positive for the markers: BEX1, CDH6, COL21A1, CXADR, EGR2, FOXF1, FST, HSD17B2,
LAMC2, MMP1, MSX1, MSX2, SERPINA3, ZIC1 and ZIC2 and is negative for the markers: ACTC, ALDH1A1,
AQP1, ATP8B4, CFB, C3, C7, C20orf103, CDH3, CLDN11, CNTNAP2, COMP, CRIP1, CRLF1, DIO2, METTL7A,
DKK2, DLK1, DPT, FGFR3, FMO1, FMO3, GABRB1, GJB2, GSC, HOXA5, HSD11B2, HSPA6, HSPB3, ICAM5,
ID4, IFI27, IGF2, KCNMB1, KIAA0644, KRT14, KRT17, MFAP5, MASP1, MEOX1, MEOX2, MGP, MX1, MYBPH,
MYH3, MYH11, NPAS1, OGN, OLR1, PAX2, PAX9, PDE1A, PRG4, PROM1, PRRX2, PTN, PTPRN, RARRES1,
RASD1, RGS1, S100A4, SFRP2, SMOC1, SNAP25, STMN2, TAC1, TNFSF7, TUBB4 and WISP2.
The cell line SK15 is positive for the markers: AREG, BEX1, FOXF1, KRT19, LAMC2, MSX1, PITX2, S100A4,
SERPINA3 and THY1 and is negative for the markers: AGC1, ALDH1A1, AQP1, ATP8B4, CFB, C3, C7, C20orf103,
CD24, CDH3, CDH6, CLDN11, CNTNAP2, COL15A1, COMP, CRIP1, CRLF1, DLK1, DPT, FMO1, FMO3,
GABRB1, GDF10, GJB2, GSC, HOXA5, HSD11B2, HSD17B2, HSPA6, HSPB3, ICAM5, ID4, IGF2, IGFBP5,
KCNMB1, KIAA0644, KRT14, KRT17, MFAP5, MASP1, MEOX1, MEOX2, MGP, MSX2, MX1, MYBPH, MYH3,
MYH11, OGN, OLR1, PAX2, PAX9, PDE1A, PRG4, PROM1, PRRX2, PTN, RARRES1, RGS1, SFRP2, SMOC1,
SNAP25, STMN2, TAC1, TNNT2, TRH, TUBB4, WISP2, ZIC1 and ZIC2.
The cell line W8Rep2a is positive for the markers: AQP1, AREG, BEX1, CDH6, COL21A1, COP1, DIO2, METTL7A,
DLK1, FMO1, FMO3, FOXF1, FOXF2, MMP1, MSX1, MSX2, PDE1A, PRRX2, SERPINA3, SNAP25, SOX11, TFPI2
and ZIC2 and is negative for the markers: ALDH1A1, ATP8B4, C3, C7, C20orf103, CD24, CLDN11, CNTNAP2,
COMP, CRIP1, CRLF1, CXADR, DKK2, DPT, EGR2, GAP43, GDF10, GJB2, GSC, HOXA5, HSD11B2, HSD17B2,
HSPA6, HSPB3, ICAM5, ID4, IFI27, KCNMB1, KRT14, KRT17, KRT34, MFAP5, MASP1, MEOX1, MEOX2, MGP,
MX1, MYBPH, MYH3, MYH11, NPAS1, NPPB, OLR1, PAX2, PAX9, PITX2, PRG4, PROM1, PRRX1, PTGS2, PTN,
PTPRN, RGS1, SFRP2, STMN2, TAC1, THY1, TNNT2, TRH, TUBB4 and ZIC1.
The cell line E55 is positive for the markers: AKR1C1, BEX1, CDH6, COL21A1, DIO2, DKK2, EGR2, GAP43, KRT19,
MSX2, PRRX1, S100A4, SOX11, THY1, TNNT2 and ZIC2 and is negative for the markers: ALDH1A1, AQP1, AREG,
ATP8B4, C3, C7, C20orf103, CLDN11, CNTNAP2, COMP, CRLF1, CXADR, DLK1, DPT, FMO1, FMO3, FOXF2,
GABRB1, GDF10, GJB2, GSC, HOXA5, HSD11B2, HSD17B2, HSPA6, HSPB3, IFI27, IGF2, KRT14, KRT34,
LAMC2, MFAP5, MASP1, MEOX1, MEOX2, MGP, MYBPH, MYH3, NPAS1, NPPB, OGN, OLR1, PAX2, PAX9,
PDE1A, PENK, PITX2, PRG4, PROM1, PRRX2, PTN, PTPRN, RARRES1, RGS1, SFRP2, SMOC1, SNAP25,
STMN2, TAC1, TRH, TUBB4, WISP2 and ZIC1.
The cell line T20 is positive for the markers: ACTC, AKR1C1, BEX1, CDH6, COL21A1, CRYAB, DKK2, EGR2,
GAP43, LAMC2, MMP1, MSX2, PITX2, SOX11, THY1 and ZIC2 and is negative for the markers: ALDH1A1, AREG,
ATP8B4, CFB, C3, C7, C20orf103, CD24, CDH3, CLDN11, CNTNAP2, COMP, CRLF1, METTL7A, DPT, FMO1,
FMO3, FOXF2, GDF10, GJB2, GSC, HOXA5, HSD11B2, HSD17B2, HSPA6, HSPB3, ICAM5, IFI27, IGF2,
KIAA0644, KRT14, MASP1, MEOX2, MGP, MX1, MYBPH, MYH3, TAGLN3, NPAS1, NPPB, OGN, OLR1, PAX2,
PDE1A, PRG4, PROM1, PRRX2, PTN, PTPRN, RARRES1, RASD1, RGS1, SFRP2, SMOC1, SNAP25, STMN2,
TAC1, TFPI2, TNFSF7, TRH, TUBB4, WISP2 and, ZIC1.
The cell line X4D20.8 is positive for the markers: BEX1, CDH6, CNTNAP2, COL21A1, CRIP1, CRYAB, DIO2, DKK2,
GAP43, ID4, LAMC2, MMP1, MSX2, S100A4, SOX11 and THY1 and is negative for the markers: AGC1, ALDH1A1,
AREG, ATP8B4, CFB, C3, C7, C20orf103, CDH3, CLDN11, COP1, CRLF1, DLK1, DPT, FMO1, FMO3, GDF10,
GJB2, GSC, HOXA5, HSD11B2, HSD17B2, HSPA6, HSPB3, ICAM5, IFI27, IGF2,
KRT14, KRT17, KRT34, MASP1,
MEOX2, MSX1, MX1, MYBPH, MYH3, MYH11, TAGLN3, NPAS1, NPPB, OGN, OLR1, PAX2, PDE1A, PRG4,
PROM1, PTN, PTPRN, RARRES1, RGS1, SNAP25, STMN2, TAC1, TNNT2, TRH, TUBB4, WISP2, ZIC1 and ZIC2.
The cell line X4D20.3 is positive for the markers: ACTC, AKR1C1, AQP1, BEX1, CDH6, COL21A1, CRYAB, DKK2,
DLK1, GJB2, HSD17B2, KRT17, LAMC2, MYL4, PITX2, S100A4, SOX11, THY1, TNNT2, ZIC1 and ZIC2 and is
negative for the markers: AGC1, ALDH1A1, AREG, ATP8B4, CFB, C3, C7, C20orf103, CDH3, CLDN11, CNTNAP2,
COMP, COP1, CRLF1, METTL7A, DPT, FGFR3, FMO1, FMO3, FOXF1, GABRB1, GSC, HOXA5, HSD11B2,
HSPA6, HSPB3, ICAM5, ID4, IFI27, IGF2, IGFBP5, KIAA0644, KRT14, KRT34, MASP1, MEOX2, MGP, MSX2,
MX1, MYBPH, MYH3, MYH11, NPAS1, OGN, OLR1, PAX9, PDE1A, PENK, PRG4, PROM1, PRRX2, PTN,
RARRES1, RGS1, SFRP2, SNAP25, STMN2, TAC1, TRH, TUBB4 and WISP2.
The cell line E132 is positive for the markers: ACTC, AKR1C1, AQP1, CD24, CDH6, COL21A1, CRYAB, DKK2,
KRT19, TAGLN3, RELN, S100A4, SFRP2, SOX11, THY1 and ZIC2 and is negative for the markers: AGC1,
ALDH1A1, AREG, ATP8B4, CFB, C3, C7, C20orf103, CLDN11, CNTNAP2, COL15A1, COMP, COP1, CRLF1,
DIO2, METTL7A, DLK1, DPT, FMO1, FMO3, FOXF1, FOXF2, FST, GABRB1, GDF10, GJB2, GSC, HOXA5,
HSD11B2, HSD17B2, HSPA6, HSPB3, ID4, IFI27, IGF2, KCNMB1, KRT14, MFAP5, MASP1, MEOX2, MGP,
MYBPH, MYH3, MYH11, NPAS1, NPPB, OGN, OLR1, PDE1A, PRG4, PROM1, PRRX2, PTGS2, PTN, PTPRN,
RARRES1, RASD1, RGS1, SERPINA3, SMOC1, SNAP25, STMN2, TAC1, TRH, TUBB4, WISP2 and ZIC1.
The cell line M13 is positive for the markers: ACTC, ANXA8, BEX1, CDH6, COL15A1, EGR2, GDF10, GJB2, KRT19,
LAMC2, MYL4, TAGLN3, S100A4, SFRP2, SOX11, THY1, ZIC1 and ZIC2 and is negative for the markers:
ALDH1A1, AREG, ATP8B4, CFB, C3, C7, C20orf103, CDH3, CLDN11, CNTNAP2, COMP, COP1, CRLF1, DIO2,
DLK1, DPT, FGFR3, FMO1, FMO3, FOXF1, GABRB1, GAP43, GSC, HOXA5, HSD11B2, HSD17B2, HSPA6,
HSPB3, ICAM5, ID4, IFI27, IGF2, KIAA0644, KRT14, MFAP5, MEOX2, MGP, MMP1, MSX2, MYBPH, MYH3,
NPAS1, OGN, OLR1, PDE1A, PRELP, PRG4, PROM1, PRRX2, PTN, PTPRN, RARRES1, RASD1, RELN, RGS1,
SMOC1, SNAP25, STMN2, TAC1, TRH, TUBB4 and WISP2.
The cell line M10 is positive for the markers: ACTC, BEX1, CDH6, COL21A1, DIO2, DKK2, EGR2, IGFBP5, PRRX1,
S100A4, SFRP2, THY1 and ZIC2 and is negative for the markers: AKR1C1, ALDH1A1, AQP1, AREG, ATP8B4, CFB,
C3, C7, C20orf103, CD24, CDH3, CLDN11, CNTNAP2, COMP, COP1, CRLF1, CXADR, METTL7A, DPT, FMO1,
FMO3, FOXF1, GABRB1, GJB2, GSC, HOXA5, HSD11B2, HSD17B2, HSPA6, HSPB3, ICAM5, IFI27, IGF2,
KIAA0644, KRT14, MEOX1, MEOX2, MGP, MYBPH, MYH3, MYH11, TAGLN3, NPAS1, OGN, OLR1, PAX2,
PAX9, PDE1A, PITX2, PRG4, PROM1, PRRX2, PTN, PTPRN, RELN, RGS1, SERPINA3, SMOC1, SNAP25, STMN2,
TAC1, TNFSF7, TNNT2, TRH, TUBB4, WISP2 and ZIC1.
The cell line E109 is positive for the markers: ACTC, AKR1C1, BEX1, CDH6, COL15A1, COL21A1, CRIP1, CRYAB,
DIO2, DKK2, GAP43, GDF5, ID4, KRT14, KRT19, KRT34, MFAP5, MEOX2, MGP, MMP1, MYH11, S100A4,
TFPI2, THY1 and ZIC1 and is negative for the markers: ALDH1A1, AQP1, AREG, ATP8B4, C3, C7, C20orf103, CD24,
CDH3, CLDN11, CNTNAP2, COMP, CRLF1, CXADR, METTL7A, DLK1, DPT, FMO1, FMO3, FOXF1, FOXF2,
GDF10, GJB2, GSC, HSD11B2, HSD17B2, HSPA6, ICAM5, IGF2, KIAA0644, MASP1, MEOX1, MYBPH, MYH3,
TAGLN3, NPAS1, NPPB, OGN, PAX2, PAX9, PDE1A, PITX2, PRG4, PROM1, PRRX2, PTN, RARRES1, RASD1,
RGS1, SFRP2, SMOC1, SNAP25, STMN2, TAC1, TRH, TUBB4 and WISP2.
The cell line E34 is positive for the markers: ACTC, AGC1, AQP1, CDH6, COL15A1, COL21A1, CRYAB, DKK2,
GAP43, KRT14, KRT17, KRT19, KRT34, MFAP5, MEOX1, MEOX2, MGP, MYH11, TAGLN3, S100A4, THY1,
TNNT2, ZIC1 and ZIC2 and is negative for the markers: ALDH1A1, AREG, ATP8B4, C3, C7, C20orf103, CDH3,
CLDN11, CNTNAP2, COMP, COP1, CRLF1, CXADR, DIO2, METTL7A, DPT, FMO1, FMO3, FOXF1, FOXF2, FST,
GABRB1, GDF10, GJB2, GSC, HOXA5, HSD11B2, HSPA6, IFI27, IGF2, KIAA0644, LAMC2, MASP1, MSX2, MX1,
MYBPH, MYH3, NPAS1, OLR1, PAX9, PDE1A, PRG4, PROM1, PRRX2, PTN, RARRES1, RASD1, RGS1,
SERPINA3, SFRP2, SMOC1, SNAP25, STMN2, TAC1, TFPI2, TRH, TUBB4 and WISP2.
The cell line E122 is positive for the markers: ACTC, AGC1, AKR1C1, BEX1, CDH6, COL21A1, CRIP1, CRYAB,
DIO2, DKK2, GAP43, ID4, KRT19, MFAP5, MYH11, MYL4, OGN, PRRX1, PTGS2, S100A4, SOX11 and THY1 and
is negative for the markers: ALDH1A1, AREG, ATP8B4, CFB, C3, C7, C20orf103, CD24, CDH3, CLDN11,
CNTNAP2, COL15A1, COP1, CRLF1, METTL7A, DLK1, DPT, FMO1, FMO3, FOXF2, GABRB1, GDF10, GJB2,
GSC, HOXA5, HSD11B2, HSD17B2, HSPA6, HSPB3, ICAM5, IFI27, IGF2, KIAA0644, KRT14, KRT17, KRT34,
LAMC2, MASP1, MEOX1, MEOX2, MYBPH, NPAS1, NPPB, OLR1, PAX2, PAX9, PDE1A, PRG4, PROM1,
RARRES1, RASD1, RGS1, SERPINA3, SFRP2, SMOC1, SNAP25, STMN2, TAC1, TUBB4, WISP2 and ZIC2.
The cell line E65 is positive for the markers: ACTC, AKR1C1, AQP1, BEX1, CD24, CDH6, COL21A1, CRYAB,
DKK2, GAP43, KRT17, KRT19, KRT34, TAGLN3, RELN, S100A4, SFRP2, SOX11, THY1 and ZIC2 and is negative
for the markers: AGC1, ALDH1A1, ATP8B4, CFB, C3, C7, C20orf103, CDH3, CLDN11, CNTNAP2, COMP, COP1,
CRIP1, CRLF1, CXADR, METTL7A, DLK1, DPT, FMO1, FMO3, FOXF2, FST, GABRB1, GDF10, GJB2, GSC,
HOXA5, HSD11B2, HSD17B2, HSPA6, HSPB3, ICAM5, IFI27, IGF2, KIAA0644, KRT14, MFAP5, MASP1, MEOX2,
MGP, MYBPH, MYH3, NPAS1, OGN, OLR1, PAX9, PDE1A, PITX2, PRG4, PROM1, PRRX2, PTGS2, PTN,
RARRES1, RASD1, RGS1, SMOC1, SNAP25, STMN2, TAC1, TRH, TUBB4, WISP2 and ZIC1.
The cell line E76 is positive for the markers: ACTC, BEX1, COL21A1, CRIP1, CRYAB, DIO2, DKK2, EGR2, GAP43,
KRT17, KRT19, MMP1, MSX2, PTGS2, S100A4 and THY1 and is negative for the markers: ALDH1A1, AREG,
ATP8B4, CFB, C3, C7, C20orf103, CDH3, CLDN11, CNTNAP2, COP1, CRLF1, METTL7A, DPT, FMO1, FMO3,
FOXF1, GABRB1, GDF10, GJB2, GSC, HOXA5, HSD11B2, HSD17B2, HSPA6, ICAM5, IFI27, IGF2, KRT14,
MBOX2, MGP, MYBPH, MYH3, NPAS1, NPPB, OGN, OLR1, PAX2, PAX9, PDE1A, PENK, PITX2, PRG4, PROM1,
PRRX2, PTN, PTPRN, RARRES1, RGS1, SFRP2, SMOC1, SNAP25, STMN2, TAC1, TFPI2, TNNT2, TRH, TUBB4,
WISP2 and ZIC1.
The cell line E108 is positive for the markers: ACTC, BEX1, CDH6, COL21A1, CRIP1, CRYAB, DIO2, DKK2,
IGFBP5, KRT17, KRT19, MYH11, S100A4, SOX11, THY1 and ZIC2 and is negative for the markers: ALDH1A1,
AQP1, AREG, ATP8B4, CFB, C3, C7, C20orf103, CD24, CDH3, CLDN11, CNTNAP2, COMP, COP1, CRLF1,
CXADR, METTL7A, DLK1, DPT, FMO1, FMO3, FOXF1, FOXF2, GABRB1, GDF10, GJB2, GSC, HOXA5,
HSD11B2, HSD17B2, HSPA6, ICAM5, IFI27, IGF2, KRT14, KRT34, MASP1, MEOX1, MEOX2, MGP, MYBPH,
MYH3, NPAS1, NPPB, OGN, OLR1, PAX2, PAX9, PDE1A, PRG4, PROM1, PTN, PTPRN, RARRES1, RASD1,
RGS1, SERPINA3, SFRP2, SMOC1, SNAP25, STMN2, TAC1, TFPI2, TNNT2, TRH, TUBB4 and WISP2.
The cell line E85 is positive for the markers: ACTC, BEX1, CDH6, COL21A1, CRYAB, DIO2, DKK2, EGR2, FGFR3,
ID4, KRT17, KRT19, MFAP5, MGP, MMP1, MYH11, PRELP, S100A4, SOX11, THY1, TNNT2, ZIC1 and ZIC2 and is
negative for the markers: ALDH1A1, AQP1, AREG, ATP8B4, CFB, C3, C7, C20orf103, CD24, CDH3, CNTNAP2,
COMP, COP1, CRLF1, METTL7A, DPT, FMO1, FMO3, GABRB1, GDF5, GDF10, GJB2, GSC, HOXA5, HSD11B2,
HSD17B2, HSPA6, ICAM5, IFI27, IGF2, KRT14, MASP1, MEBOX1, MEOX2, MYBPH, MYH3, NPAS1, OGN, OLR1,
PAX9, PDE1A, PITX2, PRG4, PROM1, PRRX2, PTN, RARRES1, RASD1, RGS1, SFRP2, SMOC1, STMN2, TAC1,
TFPI2, TRH, TUBB4 and WISP2.
The cell line M11 is positive for the markers: BEX1, CDH6, COL21A1, CRYAB, DKK2, GAP43, ID4, MMP1, MYH11,
SOX11, THY1 and ZIC1 and is negative for the markers: AGC1, ALDH1A1, AREG, ATP8B4, C3, C7, C20orf103,
CD24, CDH3, CLDN11, CNTNAP2, COMP, COP1, CRLF1, CXADR, METTL7A, DLK1, DPT, FMO1, FMO3,
FOXF2, FST, GABRB1, GDF10, GJB2, GSC, HOXA5, HSD11B2, HSD17B2, HSPA6, ICAM5, IGF2, IGFBP5,
KCNMB1, KIAA0644, KRT14, MASP1, MEOX1, MEOX2, MSX2, MX1, MYBPH, MYH3, TAGLN3, NPAS1, OLR1,
PAX2, PAX9, PDE1A, PENK, PITX2, PRG4, PROM1, PRRX2, PTN, PTPRN, RARRES1, RELN, RGS1, SFRP2,
SMOC1, SNAP25, STMN2, TAC1, TFPI2, TNFSF7, TNNT2, TRH, TUBB4, WISP2 and ZIC2.
The cell line E8 is positive for the markers: ACTC, BEX1, CDH6, COL21A1, CRIP1, CRYAB, DIO2, DKK2, ID4
KCNMB1, KRT14, KRT17, KRT19, KRT34, MFAP5, MGP, MYH11, PTGS2, S100A4, SOX11 and THY1 and is
negative for the markers: ALDH1A1, AREG, ATP8B4, C3, C7, C20orf103, CDH3, CNTNAP2, COMP, COP1, CXADR,
METTL7A, DPT, FMO1, FMO3, FOXF1, FOXF2, GABRB1, GDF10, GJB2, GSC, HOXA5, HSD11B2, HSD17B2,
HSPA6, HSPB3, ICAM5, IFI27, IGF2, IGFBP5, KIAA0644, LAMC2, MASP1, MEOX1, MSX2, MX1, MYBPH,
TAGLN3, NPAS1, NPPB, OLR1, PAX2, PAX9, PDE1A, PRELP, PRG4, PROM1, PRRX2, PTN, PTPRN, RARRES1,
RASD1, RGS1, SFRP2, SMOC1, SNAP25, STMN2, TAC1, TFPI2, TNFSF7, TRH, WISP2, ZIC1 and ZIC2.
The cell line E80 is positive for the markers: ACTC, BEX1, CDH6, COL21A1, CRYAB, DKK2, ID4, KRT19, MMP1,
MYH11, TAGLN3, SOX11 and THY1 and is negative for the markers: ALDH1A1, AQP1, AREG, ATP8B4, CFB, C3,
C7, C20orf103, CDH3, CLDN11, CNTNAP2, COMP, CRIP1, CRLF1, METTL7A, DLK1, DPT, FMO1, FMO3,
FOXF1, FOXF2, GABRB1, GDF10, GSC, HOXA5, HSD11B2, HSD17B2, HSPA6, ICAM5, IFI27, IGF2, KIAA0644,
KRT14, KRT34, MASP1, MEOX2, MGP, MYBPH, MYH3, NPAS1, OGN, OLR1, PAX9, PDE1A, PRELP, PRG4,
PROM1, PRRX2, PTN, RARRES1, RASD1, RGS1, SERPINA3, SMOC1, SNAP25, STMN2, TAC1, TNNT2, TRH,
WISP2, ZIC1 and ZIC2.
The cell line RA.D20.24 is positive for the markers: ACTC, BEX1, CRYAB, CXADR, DKK2, FOXF1, GAP43,
HOXA5, IGFBP5, KRT19, LAMC2, MFAP5, MMP1, MSX1, MYL4, PITX2, PTGS2, RELN, THY1 and TNNT2 and is
negative for the markers: AGC1, ALDH1A1, AQP1, AREG, ATP8B4, CFB, C7, C20orf103, CDH3, CNTNAP2,
COL15A1, COMP, COP1, CRLF1, DLK1, DPT, FGFR3, FMO1, FMO3, FOXF2, GDF10, GJB2, GSC, HSD11B2,
HSD17B2, HSPA6, HSPB3, ICAM5, ID4, IFI27, IGF2, KCNMB1, KRT14, MASP1, MEOX1, MEOX2, MGP, MSX2,
MX1, MYBPH, MYH3, MYH11, NPAS1, OGN, PAX2, PAX9, PDE1A, PRG4, PROM1, PRRX2, PTN, PTPRN,
RARRES1, RGS1, SFRP2, SMOC1, SNAP25, STMN2, TAC1, TUBB4, WISP2, ZIC1 and ZIC2.
The cell line RA.D20.6 is positive for the markers: ACTC, CRYAB, CXADR, DKK2, FOXF1, GAP43, HOXA5,
IGFBP5, KRT19, LAMC2, MFAP5, MMP1, MSX1, PITX2, PTGS2, SOX11 and THY1 and is negative for the markers:
ALDH1A1, ATP8B4, CFB, C3, C7, C20orf103, CDH3, CNTNAP2, COL15A1, COMP, COP1, CRLF1, DIO2, DLK1,
DPT, FMO1, FMO3, FOXF2, GDF10, GSC, HSD11B2, HSD17B2, HSPA6, HSPB3, ICAM5, ID4, IGF2, KRT14,
MASP1, MEOX1, MEOX2, MGP, MSX2, MX1, MYBPH, MYH3, MYH11, NPAS1, OGN, PAX2, PAX9, PDE1A,
PRG4, PROM1, PRRX2, PTN, PTPRN, RARRES1, RGS1, SERPINA3, SFRP2, SMOC1, STMN2, TAC1, TRH,
TUBB4, WISP2, ZIC1 and ZIC2.
The cell line RA.SMO10 is positive for the markers: ALDH1A1, BEX1, C3, CDH3, COL21A1, CXADR, METTL7A,
EGR2, FMO3, FOXF1, HOXA5, KIAA0644, MGP, RARRES1, SOX11 and STMN2 and is negative for the markers:
ACTC, AGC1, ANXA8, AQP1, CFB, C7, C20orf103, CD24, CDH6, CNTNAP2, COL15A1, COMP, COP1, CRIP1,
CRLF1, DPT, FOXF2, GAP43, GDF10, GSC, HSD11B2, HSD17B2, HSPA6, HSPB3, ICAM5, ID4, IFI27, KRT14,
KRT17, KRT34, MASP1, MEOX1, MEOX2, MMP1, MSX2, MYBPH, MYH3, MYH11, TAGLN3, NPAS1, NPPB,
OGN, PAX2, PAX9, PDE1A, PITX2, PRELP, PRG4, PROM1, PRRX2, PTN, PTPRN, RGS1, S100A4, SERPINA3,
SFRP2, SMOC1, TAC1, TFPI2, THY1, TNFSF7, TRH, TUBB4, WISP2, ZIC1 and ZIC2.
The cell line RA.SMO14 is positive for the markers: ACTC, BEX1, CD24, CXADR, FOXF1, GDF5, GJB2, HOXA5,
IGFBP5, KRT19, LAMC2, MFAP5, MMP1, RELN, SOX11 and STMN2 and is negative for the markers: AGC1,
ALDH1A1, AQP1, ATP8B4, CFB, C3, C7, CDH6, CLDN11, CNTNAP2, COL15A1, COL21A1, COMP, COP1, CRIP1,
CRLF1, DIO2, DLK1, DPT, FGFR3, FMO1, FMO3, FOXF2, GABRB1, GDF10, GSC, HSD11B2, HSD17B2, HSPA6,
HSPB3, ICAM5, ID4, IFI27, IGF2, KCNMB1, KRT14, KRT17, KRT34, MASP1, MEOX1, MEOX2, MGP, MSX2,
MYBPH, MYH3, MYH11, NPAS1, NPPB, OGN, PAX2, PAX9, PDE1A, PITX2, PRELP, PRG4, PROM1, PRRX1,
PRRX2, PTN, PTPRN, RGS1, SERPINA3, SFRP2, SMOC1, TAC1, TNFSF7, TUBB4, WISP2, ZIC1 and ZIC2.
The cell line RA.PEND18 is positive for the markers: C3, CDH3, COL21A1, METTL7A, DLK1, EGR2, FOXF1,
GABRB1, HOXA5, IGF2, KIAA0644, KRT19, MSX1, PITX2, PROM1, PTGS2, SNAP25 and SOX11 and is negative
for the markers: ACTC, AGC1, ALDH1A1, AQP1, BEX1, CFB, C20orf103, CDH6, CNTNAP2, COL15A1, COMP,
CRIP1, CRLF1, CXADR, DPT, FMO1, FOXF2, GAP43, GDF10, GSC, HSD11B2, HSD17B2, HSPA6, HSPB3,
ICAM5, ID4, IFI27, KCNMB1, KRT14, KRT34, MFAP5, MASP1, MEOX1, MEOX2, MGP, MMP1, MSX2, MYBPH,
MYH3, MYH11, TAGLN3, NPAS1, NPPB, PAX2, PAX9, PENK, PRELP, PRG4, PRRX2, PTN, PTPRN, RARRES1,
RELN, RGS1, SFRP2, SMOC1, STMN2, TAC1, TNFSF7, TRH, TUBB4, WISP2, ZIC1 and ZIC2.
The cell line RA.PEND10 is positive for the markers: AREG, C3, CDH3, CDH6, COL21A1, METTL7A, DLK1, EGR2,
FOXF1, FST, GDF5, HOXA5, IGF2, IGFBP5, KRT19, PDE1A, PITX2, RELN and SOX11 and is negative for the
markers: ACTC, AGC1, ALDH1A1, ATP8B4, CFB, C7, C20orf103, CLDN11, CNTNAP2, COL15A1, COMP, CRIP1,
CRLF1, CRYAB, DPT, FOXF2, GAP43, GDF10, GSC, HSD11B2, HSD17B2, HSPA6, HSPB3, ICAM5, ID4, IFI27,
KCNMB1, KRT14, KRT17, KRT34, MASP1, MEOX1, MEOX2, MMP1, MSX2, MYBPH, MYH3, MYH11, TAGLN3,
NPAS1, NPPB, OGN, PAX2, PAX9, PRELP, PRG4, PROM1, PRRX1, PRRX2, PTN, PTPRN, RGS1, S100A4,
SERPINA3, SFRP2, SMOC1, STMN2, TAC1, THY1, TNFSF7, TRH, TUBB4, WISP2, ZIC1 and ZIC2.
The cell line RA.SKEL21 is positive for the markers: AREG, BEX1, C3, CD24, COL21A1, COP1, METTL7A, FOXF1,
KRT19, MSX1, PITX2, SERPINA3, SOX11 and THY1 and is negative for the markers: ACTC, AGC1, ALDH1A1,
AQP1, ATP8B4, CFB, C7, C20orf103, CDH6, CLDN11, CNTNAP2, COL15A1, COMP, CRIP1, CRLF1, DKK2, DPT,
FGFR3, FMO1, FMO3, FOXF2, GAP43, GDF10, GSC, HSD11B2, HSD17B2, HSPA6, HSPB3, ICAM5, ID4, IFI27,
KCNMB1, KRT14, KRT17, KRT34, MASP1, MEOX1, MEOX2, MGP, MMP1, MSX2, MX1, MYBPH, MYH3,
TAGLN3, NPAS1, NPPB, OGN, OLR1, PAX2, PAX9, PDE1A, PENK, PRELP, PRG4, PRRX2, PTGS2, PTN, PTPRN,
RARRES1, RASD1, RELN, RGS1, SFRP2, SMOC1, STMN2, TAC1, TNFSF7, TRH, TUBB4 and ZIC2.
The cell line RA.SKEL18Rep2a is positive for the markers: AREG, C3, CD24, CDH3, COL21A1, METTL7A, DPT,
GJB2, SERPINA3, SNAP25 and SOX11 and is negative for the markers: ALDH1A1, ATP8B4, CFB, C7, C20orf103,
CDH6, CLDN11, CNTNAP2, COMP, COP1, CRIP1, DIO2, DKK2, DLK1, FGFR3, FMO1, FMO3, GDF10, GSC,
HSD11B2, HSD17B2, HSPA6, HSPB3, ICAM5, ID4, IFI27, IGF2, KCNMB1, KRTL4, KRT17, KRT19, KRT34,
MASP1, MEOX1, MEOX2, MGP, MMP1, MSX2, MYBPH, MYH3, MYH11, TAGLN3, NPAS1, NPPB, OGN, OLR1,
PAX2, PAX9, PRELP, PRG4, PROM1, PRRX1, PRRX2, PTGS2, PTN, PTPRN, RARRES1, RELN, RGS1, SFRP2,
SMOC1, STMN2, TAC1, THY1, TNFSF7, TNNT2, TRH, WISP2, ZIC1 and ZIC2.
The cell line C4.4 is positive for the markers: AKR1C1, BEX1, CDH6, COP1, DIO2, METTL7A, DKK2, DPT, EGR2,
FOXF1, FST, KIAA0644, MMP1, MSX1, RELN, S100A4, TAC1 and THY1 and is negative for the markers: AGC1,
ALDH1A1, ANXA8, AQP1, AREG, ATP8B4, CFB, C3, C7, C20orf103, CD24, CDH3, CLDN11, CNTNAP2,
COL21A1, COMP, CRIP1, CRLF1, CXADR, FGFR3, FMO1, GAP43, GDF10, GJB2, GSC, HOXA5, HSD11B2,
HSD17B2, HSPA6, HSPB3, ICAM5, ID4, IFI27, IGF2, KCNMB1, KRT14, KRT17, KRT19, KRT34, LAMC2, MFAP5,
MASP1, MEOX1, MEOX2, MGP, MYBPH, MYH3, MYH11, TAGLN3, NPAS1, NPPB, OGN, PAX2, PAX9, PDE1A,
PENK, PITX2, PRG4, PROM1, PTGS2, PTN, PTPRN, RARRES1, RASD1, RGS1, SERPINA3, SFRP2, SMOC1,
SNAP25, STMN2, TNFSF7, TNNT2, TRH, TUBB4, ZIC1 and ZIC2.
The cell line W7 is positive for the markers: AREG, C3, COL15A1, COL21A1, COP1, CXADR, DIO2, DLK1, EGR2,
FMO1, FOXF1, GDF5, HOXA5, KIAA0644, METTL7A, PITX2, PROM1, S100A4, SERPINA3 and SOX11 and is
negative for the markers: AGC1, ALDH1A1, AQP1, ATP8B4, C20orf103, C7, CD24, CDH3, CDH6, CFB, CLDN11,
CNTNAP2, COMP, CRIP1, DKK2, DPT, FMO3, GABRB1, GAP43, GDF10, GSC, HSD11B2, HSD17B2, HSPA6,
ICAM5, ID4, IFI27, KCNMB1, KRT14, KRT17, KRT19, KRT34, MASP1, MEOX1, MEOX2, MGP, MMP1, MYBPH,
MYH11, MYH3, NPAS1, NPPB, OGN, PAX2, PAX9, PRG4, PRRX2, PTN, PTPRN, RARRES1, RASD1, RELN,
RGS1, SFRP2, SMOC1, STMN2, TAC1, TNFSF7, TRH, TUBB4, ZIC1 and ZIC2.
The cell line X4SKEL20 is positive for the markers: AREG, BEX1, C3, C7, COP1, CXADR, FOXF1, FST, KRT19,
METTL7A, MGP, MSX1, PITX2, SERPINA3 and TFPI2 and is negative for the markers: ALDH1A1, AQP1, ATP8B4,
C20orf103, CD24, CDH3, CDH6, CFB, CLDN11, CNTNAP2, COL15A1, COMP, DKK2, DLK1, DPT, EGR2, FGFR3,
FMO1, FOXF2, GABRB1, GAP43, GDF10, GDF5, GJB2, GSC, HOXA5, HSD11B2, HSD17B2, HSPA6, HSPB3,
ICAM5, ID4, IFI27, IGF2, IGFBP5, KCNMB1, KRT14, KRT34, MASP1, MEOX1, MEOX2, MFAP5, MMP1, MSX2,
MX1, MYBPH, MYH11, MYH3, NPAS1, NPPB, OGN, OLR1, PAX2, PENK, PRG4, PROM1, PRRX1, PRRX2, PTN,
PTPRN, RARRES1, RELN, RGS1, SFRP2, SMOC1, SOX11, STMN2, TAC1, TAGLN3, THY1, TNFSF7, TNNT2,
TRH, WISP2, ZIC1 and ZIC2.
The cell line C4ELSR6 is positive for the markers: ACTC, BEX1, C7, CDH6, COL21A1, DIO2, METTL7A, DKK2,
FOXF1, FOXF2, LAMC2, PITX2, PRRX1, S100A4, SFRP2, SNAP25, SOX11, TAC1 and TFPI2 and is negative for the
markers: AGC1, ALDH1A1, AREG, ATP8B4, CFB, C3, C20orf103, CD24, CLDN11, CNTNAP2, COMP, CRIP1,
CRLF1, CRYAB, DLK1, DPT, FGFR3, FMO3, GAP43, GDF5, GDF10, GJB2, GSC, HOXA5, HSD11B2, HSD17B2,
HSPA6, HSPB3, ICAM5, ID4, IFI27, IGF2, KCNMB1, KRT14, KRT17, KRT34, MFAP5, MASP1, MEOX1, MEOX2,
MGP, MMP1, MYBPH, MYH3, MYH11, NPAS1, NPPB, PAX2, PAX9, PENK, PRG4, PTN, PTPRN, RARRES1,
RASD1, RGS1, SMOC1, STMN2, TNFSF7, TRH, TUBB4, WISP2 and ZIC1.
The cell line J2 is positive for the markers: ACTC, AKR1C1, BEX1, CDH6, COL15A1, COL21A1, DIO2, METTL7A,
DKK2, DLK1, FOXF1, KIAA0644, MGP, PDE1A, PRRX1, SFRP2, SNAP25, TNNT2 and ZIC2 and is negative for the
markers: AGC1, ALDH1A1, ATP8B4, CFB, C3, C20orf103, CD24, CNTNAP2, COMP, CRIP1, CRLF1, DPT, FGFR3,
GABRB1, GDF10, GSC, HOXA5, HSD11B2, HSD17B2, HSPA6, ICAM5, ID4, IFI27, KCNMB1, KRT14, KRT17,
KRT19, KRT34, LAMC2, MFAP5, MASP1, MEOX1, MMP1, MSX1, MYBPH, MYH3, MYH11, NPAS1, NPPB,
OGN, OLR1, PAX2, PAX9, PENK, PROM1, PRRX2, PTN, PTPRN, RARRES1, RGS1, SMOC1, STMN2, TAC1,
TNFSF7, TRH and TUBB4.
The cell line F15 is positive for the markers: BEX1, CDH6, COL15A1, COL21A1, DKK2, DLK1, FOXF1, FST, GDF5,
KRT19, MGP, MMP1, PRRX1, SERPINA3, SNAP25, SOX11, ZIC1 and ZIC2 and is negative for the markers: ACTC,
AGC1, ALDH1A1, AQP1, AREG, ATP8B4, CFB, C3, C7, C20orf103, CD24, CDH3, CNTNAP2, COMP, CRLF1,
DIO2, DPT, FGFR3, FMO1, FMO3, FOXF2, GABRB1, GDF10, GJB2, GSC, HOXA5, HSD11B2, HSD17B2, HSPA6,
HSPB3, ICAM5, ID4, IFI27, IGF2, KCNMB1, KIAA0644, KRT14, KRT17, MASP1, MEOX1, MEOX2, MYBPH,
MYH3, MYH11, NPAS1, NPPB, OGN, OLR1, PAX2, PDE1A, PENK, PITX2, PRG4, PROM1, PRRX2, PTN, PTPRN,
RGS1, SFRP2, SMOC1, STMN2, TFPI2, TNNT2, TRH and TUBB4.
The cell line X4SKEL4 is positive for the markers: ANXA8, AREG, BEX1, C3, COL21A1, COP1, CXADR,
METTL7A, EGR2, FOXF1, FST, KRT19, LAMC2, MYL4, PITX2 and SERPINA3 and is negative for the markers:
ALDH1A1, AQP1, ATP8B4, CFB, C7, C20orf103, CD24, CDH3, CDH6, CLDN11, CNTNAP2, COL15A1, COMP,
CRLF1, DKK2, DLK1, DPT, FGFR3, FMO3, FOXF2, GABRB1, GAP43, GDF5, GDF10, GJB2, GSC, HOXA5,
HSD11B2, HSD17B2, HSPA6, HSPB3, ICAM5, ID4, IFI27, IGF2, IGFBP5, KIAA0644, KRT14, KRT17, KRT34,
MASP1, MEOX1, MEOX2, MGP, MMP1, MSX2, MX1, MYBPH, MYH3, NPAS1, NPPB, OGN, OLR1, PAX2, PAX9,
PDE1A, PENK, PRELP, PRG4, PROM1, PRRX2, PTN, PTPRN, RARRES1, RASD1, RGS1, SFRP2, SMOC1, SOX11,
STMN2, TAC1, TNNT2, TRH, TUBB4, WISP2 and ZIC1.
The cell line X4SKEL19 is positive for the markers: AREG, COL21A1, COP1, DIO2, METTL7A, EGR2, FOXF1, FST,
KIAA0644, KRT19, MGP, PDE1A, PITX2, SERPINA3 and TFPI2 and is negative for the markers: ACTC, AGC1,
ALDH1A1, AQP1, ATP8B4, CFB, C20orf103, CD24, CDH3, CDH6, CLDN11, CNTNAP2, COL15A1, COMP, CRIP1,
CRLF1, CXADR, DKK2, DLK1, DPT, FGFR3, FMO1, FOXF2, GABRB1, GAP43, GDF5, GDF10, GJB2, GSC,
HOXA5, HSD11B2, HSD17B2, HSPA6, HSPB3, ICAM5, ID4, IFI27, IGF2, KCNMB1, KRT14, KRT17, KRT34,
MFAP5, MASP1, MEOX1, MEOX2, MMP1, MSX2, MX1, MYBPH, MYH3, MYH11, TAGLN3, NPAS1, NPPB,
OGN, OLR1, PAX2, PAX9, PRELP, PRG4, PRRX2, PTN, PTPRN, RELN, SFRP2, SMOC1, SOX11, STMN2, TAC1,
THY1, TRH, WISP2, ZIC1 and ZIC2.
The cell line X4SKEL8 is positive for the markers: AREG, BEX1, COL21A1, DIO2, METTL7A, DKK2, EGR2, FMO3,
FOXF1, FST, MYL4, PITX2, PTGS2, S100A4 and SERPINA3 and is negative for the markers: ALDH1A1, AQP1,
ATP8B4, CFB, C3, C20orf103, CD24, CDH3, CLDN11, CNTNAP2, COMP, CRIP1, CRLF1, DLK1, DPT, FGFR3,
FOXF2, GABRB1, GDF5, GDF10, GJB2, GSC, HOXA5, HSD11B2, HSD17B2, HSPA6, HSPB3, ICAM5, ID4, IFI27,
IGF2, KRT14, KRT17, KRT34, MFAP5, MASP1, MEOX1, MEOX2, MGP, MMP1, MSX2, MX1, MYBPH, MYH3,
MYH11, TAGLN3, NPAS1, NPPB, OGN, OLR1, PAX2, PAX9, PDE1A, PENK, PRG4, PRRX1, PRRX2, PTN,
PTPRN, RARRES1, RASD1, RELN, RGS1, SFRP2, SMOC1, STMN2, TAC1, THY1, TNFSF7, TNNT2, TRH, TUBB4,
ZIC1 and ZIC2.
The cell line RA.PEND17Bio2a is positive for the markers: AREG, BEX1, CDH6, COL15A1, COL21A1, COP1,
METTL7A, DPT, EGR2, FOXF1, FST, GJB2, LAMC2, MSX2, PTGS2, SERPINA3 and SFRP2 and is negative for the
markers: ACTC, ALDH1A1, AQP1, ATP8B4, CFB, C20orf103, CD24, CDH3, CNTNAP2, COMP, CRIP1, CXADR,
FGFR3, FMO1, GABRB1, GAP43, GDF10, GSC, HOXA5, HSD11B2, HSD17B2, HSPA6, HSPB3, ID4, IFI27, IGF2,
KCNMB1, KRT14, KRT17, KRT34, MFAP5, MASP1, MEOX1, MEOX2, MGP, MMP1, MX1, MYBPH, MYH3,
MYH11, NPAS1, NPPB, OLR1, PAX2, PAX9, PDE1A, PRELP, PRG4, PROM1, PRRX2, PTN, PTPRN, RELN, RGS1,
SMOC1, STMN2, TAC1, THY1, TNFSF7, TNNT2, TRH, TUBB4, ZIC1 and ZIC2.
The cell line W9 is positive for the markers: AKR1C1, C7, CDH6, COL21A1, METTL7A, DLK1, EGR2, FOXF1,
GDF5, GJB2, HOXA5, IGFBP5, KIAA0644, KRT19, MGP, OGN, PITX2, SERPINA3, SOX11, TFPI2 and ZIC2 and is
negative for the markers: AGC1, ALDH1A1, AQP1, CFB, C3, C20orf103, CD24, CDH3, CLDN11, CNTNAP2,
COL15A1, COMP, CRIP1, CRLF1, CRYAB, DKK2, FGFR3, FMO1, FMO3, FOXF2, GDF10, GSC, HSD11B2,
HSD17B2, HSPA6, HSPB3, ICAM5, ID4, IFI27, IGF2, KCNMB1, KRT14, KRT17, KRT34, MFAP5, MASP1,
MEOX1, MEOX2, MSX2, MX1, MYBPH, MYH3, MYH11, NPAS1, NPPB, OLR1, PAX2, PAX9, PDE1A, PENK,
PRG4, PROM1, PRRX2, PTN, PTPRN, RARRES1, RASD1, RGS1, SFRP2, SNAP25, STMN2, TAC1, THY1, TNFSF7,
TNNT2, TRH, TUBB4 and ZIC1.
The cell line MW4 is positive for the markers: AKR1C1, AREG, BEX1, C7, COL15A1, COL21A1, DIO2, METTL7A,
DKK2, EGR2, FMO3, FOXF1, FOXF2, PITX2, PRELP, SERPINA3, SFRP2 and TFPI2 and is negative for the markers:
ALDH1A1, AQP1, ATP8B4, CFB, C3, C20orf103, CD24, CDH3, CLDN11, CNTNAP2, CRIP1, CXADR, DLK1,
GABRB1, GDF5, GDF10, GJB2, GSC, HOXA5, HSD11B2, HSD17B2, HSPA6, HSPB3, ICAM5, ID4, IFI27, IGF2,
KCNMB1, KRT14, KRT17, KRT19, KRT34, MFAP5, MASP1, MEOX1, MEOX2, MGP, MMP1, MSX1, MX1,
MYBPH, MYH3, MYH11, NPAS1, NPPB, OLR1, PAX2, PAX9, PDE1A, PENK, PRG4, PROM1, PRRX1, PTN,
PTPRN, RARRES1, RELN, RGS1, SMOC1, STMN2, TAC1, TNNT2, TUBB4, ZIC1 and ZIC2,.
The cell line SK58 is positive for the markers: AKR1C1, AREG, BEX1, C7, COL15A1, COL21A1, METTL7A, EGR2,
FMO1, FOXF1, PTGS2, SERPINA3, SFRP2, TAC1 and TFPI2 and is negative for the markers: ACTC, AGC1,
ALDH1A1, AQP1, ATP8B4, CFB, C3, C20orf103, CD24, CDH3, CDH6, CLDN11, CNTNAP2, COP1, CRIP1, DIO2,
DLK1, DPT, GABRB1, GDF5, GDF10, GSC, HOXA5, HSD11B2, HSD17B2, HSPB3, ID4, IFI27, IGP2, KCNMB1,
KRT14, KRT17, KRT19, KRT34, MFAP5, MASP1, MEOX1, MEOX2, MMP1, MSX2, MX1, MYBPH, MYH3,
MYH11, NPAS1, NPPB, OLR1, PAX2, PAX9, PDE1A, PRG4, PROM1, PRRX2, PTN, PTPRN, RARRES1, RELN,
RGS1, SMOC1, STMN2, TNNT2, TRH, TUBB4, ZIC1 and ZIC2,.
The cell line SK25 is positive for the markers: BEX1, COL21A1, METTL7A, FMO1, FOXF1, LAMC2, SERPINA3,
SFRP2 and WISP2 and is negative for the markers: ACTC, ALDH1A1, ANXA8, AQP1, ATP8B4, CFB, C3, C20orf103,
CD24, CDH3, CLDN11, CNTNAP2, COMP, CRIP1, CRLF1, CXADR, DIO2, DKK2, DPT, EGR2, FGFR3, GABRB1,
GAP43, GDF10, GJB2, GSC, HOXA5, HSD11B2, HSD17B2, HSPA6, HSPB3, ICAM5, ID4, IFI27, IGF2, KCNMB1,
KIAA0644, KRT14, KRT17, KRT34, MFAP5, MASP1, MEOX1, MEOX2, MGP, MMP1, MSX2, MYBPH, MYH3,
MYH11, NPAS1, NPPB, OGN, OLR1, PAX2, PAX9, PDE1A, PITX2, PRELP, PRG4, PROM1, PTN, RARRES1,
RASD1, RGS1, SMOC1, STMN2, TAC1, TFPI2, TNFSF7, TNNT2, TRH, ZIC1 and ZIC2.
The cell line SK16 is positive for the markers: AREG, BEX1, COL15A1, COL21A1, METTL7A, EGR2, FMO1,
FOXF1, LAMC2, MSX1, PITX2, SERPINA3, ZIC1 and ZIC2 and is negative for the markers: AGC1, ALDH1A1,
AQP1, ATP8B4, CFB, C3, C20orf103, CD24, CDH3, CLDN11, CNTNAP2, COMP, CRIP1, CXADR, DIO2, DKK2,
DPT, FGFR3, GABRB1, GDF10, GSC, HSD11B2, HSD17B2, HSPA6, HSPB3, ID4, IFI27, IGF2, KIAA0644, KRT14,
KRT17, KRT19, KRT34, MFAP5, MASP1, MEOX1, MEOX2, MGP, MMP1, MSX2, MX1, MYBPH, MYH3, MYH11,
TAGLN3, NPAS1, NPPB, OLR1, PAX2, PAX9, PENK, PRELP, PRG4, PROM1, PRRX2, PTN, RARRES1, RELN,
RGS1, STMN2, TAC1, TFPI2, THY1, TNTSF7, TNNT2, TRH and TUBB4,.
The cell line EN20 is positive for the markers: BEX1, COL21A1, METTL7A, DLK1, FMO1, FOXF1, FST, GDF5,
LAMC2, MGP, PRRX1, S100A4, SERPINA3, SOX11, TFPI2 and WISP2 and is negative for the markers: ALDH1A1,
AQP1, ATP8B4, C3, C7, C20orf103, CD24, CDH3, CNTNAP2, COL15A1, COMP, CRIP1, CXADR, DIO2, DKK2,
FGFR3, GABRB1, GAP43, GDF10, GSC, HOXA5, HSD11B2, HSD17B2, HSPA6, HSPB3, ICAM5, ID4, IFI27,
KCNMB1, KRT14, KRT17, KRT34, MFAP5, MASP1, MEOX1, MEOX2, MMP1, MX1, MYBPH, MYH3, MYH11,
NPAS1, NPPB, OLR1, PAX2, PDE1A, PITX2, PRELP, PRG4, PROM1, PTN, PTPRN, RASD1, RGS1, SFRP2,
SMOC1, SNAP25, STMN2, TAC1, TNFSF7, TNNT2, TRH, TUBB4, ZIC1 and ZIC2,.
The cell line EN43 is positive for the markers: AKR1C1, BEX1, C7, CDH6, COL21A1, DIO2, METTL7A, DLK1,
FMO1, FMO3, FOXF1, FOXF2, FST, GDF5, MMP1, MSX1, OGN, PRRX1, S100A4, SERPINA3 and SOX11 and is
negative for the markers: ALDH1A1, ANXA8, AQP1, ATP8B4, C3, C20orf103, CD24, CDH3, CLDN11, CNTNAP2,
COMP, CRIP1, CRLF1, DKK2, DPT, GABRB1, GAP43, GDF10, GJB2, GSC, HOXA5, HSD11B2, HSD17B2, HSPA6,
ID4, IFI27, IGF2, KCNMB1, KRT14, KRT17, KRT19, KRT34, MFAP5, MASP1, MEOX1, MEOX2, MGP, MYBPH,
MYH3, MYH11, NPAS1, NPPB, OLR1, PAX2, PAX9, PDE1A, PITX2, PRG4, PROM1, PTN, PTPRN, RASD1, RGS1,
SFRP2, SMOC1, STMN2, THY1, TNNT2, TRH, TUBB4, ZIC1 and ZIC2.

The gene expression markers for novel human embryonic progenitor lines described herein are understood in the art to refer to RNA transcript quantitation assays that are dependent on the use of probe sequences, and the choice of probe sequence can, in the case for instance of splice variants, alter the result of the assay. Therefore, reference is made herein to the manufacturer and version number of microarrays used to determine the level of expression of genes which allows one skilled in the art to determine the associated probe sequences from the accession numbers provided herein.

The cell lines produced according to aspects of the present invention have been shown to have significant in vitro growth potential (e.g., being able to go through 20 or more doublings). As such, these populations find use in a number of research and clinical applications, some of which are described below.

The present invention uniquely describes novel methods for the in vitro production of numerous distinct populations of cells differentiated from, or in the process of differentiating from, embryonic pluripotent stem cells such as hES, hEG, hiPS, hEC, hED cells or other pluripotent embryonic stem cells such as primitive endoderm, mesoderm, or ectodermal cells. These resulting populations of cells can be documented not to have contaminating cells from the original pluripotent stem cells from which they are derived and have significant growth potential. Moreover, analysis of the gene expression patterns in these cells, as well as their growth and differentiation characteristics under different culture conditions, allows for their use in numerous applications, including for in vivo cell therapy, for the isolation of novel extracts with therapeutic or research utility, for use as induction agents for cell differentiation, and for the derivation of ligands that specifically bind to the genes expressed in the cells (e.g., cell surface receptors).

In certain embodiments, the cell populations of this invention can be used for the production of specific ligands, growth factors, differentiation factors, inhibitors, etc., that can be used in basic research applications as well as for in vivo therapies. For example, a cell population of the invention that produces significant levels of WNT may be used as a cell source to purify this factor. This can be especially important for factors that have specific modifications, e.g., lipidation, that impact the function of these factors and that are not present when they are produced in alternative cells (e.g., bacteria).

In certain embodiments, the cell populations of this invention can be used as feeder cells or inducer cells for the propagation and/or differentiation of certain cell types based on their gene expression patterns. For example, a cell that produces a specific growth or differentiation factor can be employed as a feeder cell line that will maintain a population of cells (i.e., to facilitate propagation). A cell population that produces one or more specific differentiation factors may be used to induce in-vitro differentiation of cells. When the cell population produces specific soluble factors in the culture media, culture supernatants from these cells (i.e., conditioned media) may be obtain and used to propagate/differentiate other cells.

In certain embodiments, the cell populations of the present invention can be used as model cell lines for cells specific to a developmental stage and/or location in a developing animal. For example, cell populations that exhibit gene expression patterns indicative of cells at particular developmental stages/location in an animal can be used to identify additional markers for that cell type. Regents for identifying cells expressing these genetic markers, either previously available of produced using the cell population itself, can then be employed to identify and/or isolate cells from an animal having a particular phenotype.

Moreover, populations of cells that express genes associated with specific diseases or developmental defects or conditions find use as candidates for therapeutic agents. For example, defects in the LHX8 gene, which is reported to be expressed only in the medical ganglionic eminence and perioral mesenchyme of the mouse in the middle embryonic to early postnatal development, are known to lead to cleft palate. A cell line expresses LHX8 would thus be a candidate for not only studying the activity of this gene but also as a potential therapeutic agent (see Example 51, below).

Therefore, this invention contemplates using the cells derived from the methods of this invention in a number of ways, giving them a substantial and specific credible utility. These cells (or their progeny or cell differentiated from them) may be used for research therapeutically (e.g., for transplantation purposes), for the growth factors/including agents they secrete (e.g., as purified factors or as conditioned media), as feeder cells for the derivation, production or maintenance of other cells (e.g., ES cells). The culture media from these cells may be used to induce differentiation of pluripotent stem cells in methods of this invention.

Unless otherwise defined, all technical and scientific terms used herein have the same meaning as commonly understood by one of ordinary skill in the art. In case of conflict, the present specification, including definitions, will control.

Further, unless otherwise required by context, singular terms shall include pluralities and plural terms shall include the singular.

Generally, nomenclatures used in connection with, and techniques of, cell and tissue culture, molecular biology, immunology, microbiology, genetics, developmental biology, cell biology described herein are those well-known and commonly used in the art.

Exemplary methods and materials are described below, although methods and materials similar or equivalent to those described herein can also be used in the practice or testing of the present invention.

All publications, patents, patent publications and other references mentioned herein are incorporated by reference in their entirety.

Throughout this specification and claims, the word “comprise,” or variations such as “comprises” or “comprising” will be understood to imply the inclusion of a stated integer or group of integers but not the exclusion of any other integer or group of integers.

### Biological Deposits

Cell lines described in this application have been deposited with the American Type Culture Collection (“ATCC”; P.O. Box 1549, Manassas, Va. 20108, USA) under the Budapest Treaty. The B-28 cell line, also referred to as ACTC60 or clone 17 of Series 1, was deposited On Jun. 8, 2006 and has ATCC Accession No. PTA-7654, as described in Example 21 below. The CM0-2 cell line (also known as ACTC77) was also deposited on Jun. 8, 2006 and has ATCC Accession No. PTA-7655. Another clone (cell line) described in this application, designated the Z11 cell clone, was deposited with the ATCC on Aug. 30, 2006 and has ATCC Accession No. PTA-7848. The cell line SK17, another clone (cell line) described herein, was deposited at the ATCC on Oct. 6, 2006 and has ATCC Accession No. PTA-7911. The 8-30 cell line (also known as ACTC61) of Series 1, was deposited at the ATCC on Jan. 3, 2007 and has ATCC Accession No. ______. The U31 cell line, was deposited at the ATCC on Jan. 3, 2007 and has ATCC Accession No. ______. The C5 E68 cell line, was deposited at the ATCC on Jan. 3, 2007 and has ATCC Accession No. ______.

### EXAMPLES

### Example 1

hES cells are grown to form embryoid bodies (EB) (see U.S. application Nos. 60/538,964, filed Jan. 23, 2004; Ser. No. 11/186,720, filed Jul. 20, 2005; PCT application nos. PCT/US05/002273, filed Jan. 24, 2005; PCT/US05/25860, filed Jul. 20, 2005, the disclosures of which are hereby incorporated by reference) and said embryoid bodies are plated in standard tissue culture vessels in the presence of DMEM media supplemented with 10% fetal bovine serum to obtain a heterogeneous population of cells. The media of said cultures is collected after 24 hours and the cultures are refed. The collected media are pooled, filtered through a 0.2 micron sterile filter and stored at 4° C. as conditioned medium. After a total of 10 days of differentiation, the differentiated cells are plated at limiting dilution, photographed to document the cell number in each well as well as the differentiated state of the cell, and fed the conditioned medium with biweekly refeeding, and cultured for two weeks in low ambient oxygen (5%), then microscopically analyzed for colony formation. The observed single cell-derived colonies, or clones, can then be expanded, cryopreserved, quality controlled, and their pattern of gene expression tested using gene expression arrays as is well known in the art.

In this example, colonies with a pattern of gene expression consistent with that of paraxial mesoderm and scarless skin repair are used as marker of cells useful in scarless skin repair. Alternatively, dermal fibroblasts can be isolated that express proteins for elastogenesis useful in inducing elastogenesis when transplanted in vivo.

### Example 2

hES cells are grown to form embryoid bodies (EB) (see U.S. application Nos. 60/538,964, filed Jan. 23, 2004; Ser. No. 11/186,720, filed Jul. 20, 2005; PCT application nos. PCT/US05/002273, filed Jan. 24, 2005; PCT/US05/25860, filed Jul. 20, 2005, the disclosures of which are hereby incorporated by reference) and said embryoid bodies are plated in standard tissue culture vessels in the presence of DMEM media supplemented with 10% fetal bovine serum to obtain a heterogeneous population of cells. The media of said cultures is collected after 24 hours and the cultures are refed. The collected media is pooled, filtered through a 0.2 micron sterile filter and stored at 4° C. as conditioned medium. After a total of 10 days of differentiation, the differentiated cells are plated at limiting dilution, photographed to document the cell number in each well as well as the differentiated state of the cell, and fed the conditioned medium with biweekly refeeding, and cultured for two weeks in low ambient oxygen (5%), then microscopically analyzed for colony formation. The observed single cell-derived colonies, or clones, can then be expanded, cryopreserved, quality controlled, and their pattern of gene expression tested using gene expression arrays as is well known in the art.

In this example, colonies with a pattern of gene expression consistent with that of endodermal cells are identified for use in liver cell, pancreatic beta cell, and intestinal cell transplantation.

### Example 3

hES cells are grown to form embryoid bodies (EB) (see U.S. application Nos. 60/538,964, filed Jan. 23, 2004; Ser. No. 11/186,720, filed Jul. 20, 2005; PCT application nos. PCT/US05/002273, filed Jan. 24, 2005; PCT/US05/25860, filed Jul. 20, 2005, the disclosures of which are hereby incorporated by reference) and said embryoid bodies are plated in standard tissue culture vessels in the presence of DMEM media supplemented with 10% fetal bovine serum to obtain a heterogeneous population of cells. The media of said cultures is collected after 24 hours and the cultures are refed. The collected media is pooled, filtered through a 0.2 micron sterile filter and stored at 4° C. as conditioned medium. After a total of 10 days of differentiation, the differentiated cells are plated at limiting dilution, photographed to document the cell number in each well as well as the differentiated state of the cell, and fed the conditioned medium with biweekly refeeding, and cultured for two weeks in low ambient oxygen (5%), then microscopically analyzed for colony formation. The observed single cell-derived colonies, or clones, can then be expanded, cryopreserved, quality controlled, and their pattern of, gene expression tested using gene expression arrays as is well known in the art.

In this example, colonies with a pattern of gene expression consistent with that of ectodermal cells are identified for use in neuronal, and epidermal transplantation.

### Example 4

hES cells are grown to form embryoid bodies (EB) (see U.S. application Nos. 60/538,964, filed Jan. 23, 2004; Ser. No. 11/186,720, filed Jul. 20, 2005; PCT application nos. PCT/US05/002273, filed Jan. 24, 2005; PCT/US05/25860, filed Jul. 20, 2005, the disclosures of which are hereby incorporated by reference) and said embryoid bodies are plated in standard tissue culture vessels in the presence of DMEM media supplemented with 10% fetal bovine serum to obtain a heterogeneous population of cells. The media of said cultures is collected after 24 hours and the cultures are refed. The collected media is pooled, filtered through a 0.2 micron sterile filter and stored at 4° C. as conditioned medium. After a total of 10 days of differentiation, the differentiated cells are plated at limiting dilution, photographed to document the cell number in each well as well as the differentiated state of the cell, and fed the conditioned medium with biweekly refeeding, and cultured for two weeks in low ambient oxygen (5%), then microscopically analyzed for colony formation. The observed single cell-derived colonies, or clones, can then be expanded, cryopreserved, quality controlled, and their pattern of gene expression tested using gene expression arrays as is well known in the art.

In this example, colonies with a pattern of gene expression consistent with that of cardiac progenitors, stromal fibroblasts including but not limited to cardiac, liver, pancreatic, lung, dermal, renal, AGM region, and intestinal stromal cells are used for transplantation.

### Example 5

hED cells are allowed to differentiate without forming ES cell lines and without forming embryoid bodies and are differentiated for 10 days in DMEM media supplemented with 10% fetal bovine serum to obtain a heterogeneous population of cells. The media of said cultures is collected after 24 hours and the cultures are refed. The collected media is pooled, filtered through a 0.2 micron sterile filter and stored at 4° C. as conditioned medium. After a total of 10 days of differentiation, the differentiated cells are trypsinized to form a single cell suspension, the trypsin is neutralized with serum, and the cells are incubated for 15 minutes while gently agitating cells to keep them in suspension while allowing the re-expression of cell surface antigens that may have been removed by trypsin. The cells are then sorted by flow cytometry to select cells positive for endosialin (CD248) using antibody to the antigen. CD248 positive cells and/or other cells are dispersed one cell per well in a multiwell tissue culture plate. The cells are fed the conditioned medium with biweekly refeeding, and cultured for two weeks in low ambient oxygen (5%), then microscopically analyzed for colony formation. The observed single cell-derived colonies, or clones, can then be expanded, cryopreserved, quality controlled, and their pattern of gene expression tested using gene expression arrays as is well known in the art.

In this example, the fibroblasts are used for cell induction, and for transplantation in dermal applications such as for promoting scarless wound healing.

### Example 6

hED cells are allowed to differentiate without forming ES cell lines and without forming embryoid bodies and are differentiated for 10 days in DMEM media supplemented with 10% fetal bovine serum to obtain a heterogeneous population of cells. The media of said cultures is collected after 24 hours and the cultures are refed. The collected media is pooled, filtered through a 0.2 micron sterile filter and stored at 4° C. as conditioned medium. Candidate cells differentiated for 4-8 days in 10% fetal bovine serum are trypsinized, the trypsin is neutralized. And the resulting single cell suspension is sorted by flow cytometry using techniques well known in the art using an antibody to AC4, an antigen known to sort neural crest cells. Single cells are plated at a density of a single cell per well using an automated cell deposition device (“ACDU”). The single cell-derived cultures that result are used for a number of research and therapeutic modalities that use neural crest cells, including the identification of cell cultures that display a dermal prenatal embryonic pattern of gene expression useful for transplantation into the face for regenerating elastic architecture in the dermis and for promoting scarless wound repair.

### Example 7

hED cells are allowed to differentiate without forming ES cell lines and without forming embryoid bodies and are differentiated for 10 days in DMEM media supplemented with 10% fetal bovine serum to obtain a heterogeneous population of cells. The media of said cultures is collected after 24 hours and the cultures are refed. The collected media is pooled, filtered through a 0.2 micron sterile filter and stored at 4° C. as conditioned medium. After a total of 10 days of differentiation, the differentiated cells are trypsinized to form a single cell suspension. The trypsin is then neutralized with serum. And the cells are then incubated for 15 minutes while gently agitating to keep them in suspension, while allowing the re-expression of cell surface antigens that may have been removed by trypsin. The cells are then sorted by flow cytometry to select cells positive for endosialin (CD248) using antibody to the antigen. And the CD248 positive cells and/or other cells are dispersed one cell per well in a multiwell tissue culture plate. The cells are fed the conditioned medium with biweekly refeeding, and cultured for two weeks in low ambient oxygen (5%), then microscopically analyzed for colony formation. The observed single cell-derived colonies, or clones, can then be expanded, cryopreserved, quality controlled, and their pattern of gene expression tested using gene expression arrays as is well known in the art.

In this example, the fibroblasts with a dermal progenitor pattern of gene expression are used to generate conditioned medium which is concentrated and applied topically in promoting scarless wound healing.

### Example 8

hES cells are grown to form embryoid bodies (EB) (see U.S. application Nos. 60/538,964, filed Jan. 23, 2004; Ser. No. 11/186,720, filed Jul. 20, 2005; PCT application nos. PCT/US05/002273, filed Jan. 24, 2005; PCT/US05/25860, filed Jul. 20, 2005, the disclosures of which are hereby incorporated by reference) and said embryoid bodies are plated in standard tissue culture vessels in the presence of DMEM media supplemented with 10% fetal bovine serum to obtain a heterogeneous population of cells. The media of said cultures is collected after 24 hours and the cultures are refed. The collected media is pooled; filtered through a 0.2 micron sterile filter and stored at 4° C. as conditioned medium. After a total of 10 days of differentiation, the differentiated cells are plated at limiting dilution, photographed to document the cell number in each well as well as the differentiated state of the cell, and fed the conditioned medium with biweekly refeeding, and cultured for two weeks in low ambient oxygen (5%), then microscopically analyzed for colony formation. The observed single cell-derived colonies expressing pigment, or pigmented clones, can then be expanded, cryopreserved, quality controlled, and their pattern of gene expression tested using gene expression arrays as is well known in the art.

In this example, colonies with a pattern of gene expression consistent with that of retinal pigment epithelial cells (“RPE”) are identified by examining the extracellular matrix of the cultured RPE cells for proteins of Bruch's membrane. This can be performed by techniques well known in the art, including, but not limited to, extracting the cells from the culture substrate with a detergent such as deoxycholate, and detecting the proteins that remain on said substrate using antibodies to the proteins of Bruch's membrane. The RPE cells that display a prenatal pattern of gene expression such that they deposit embryonic Bruch's membrane proteins can be identified in this manner, cryopreserved, and subsequently injected into the retina in association with degenerative diseases of the retina that have dysfunctional Bruch's membrane such that the injected RPE cells deposit new Bruch's membrane proteins and regenerate the membrane.

### Example 9

hES cells are grown to form embryoid bodies (EB) (see U.S. application Nos. 60/538,964, filed Jan. 23, 2004; Ser. No. 11/186,720, filed Jul. 20, 2005; PCT application nos. PCT/US05/002273, filed Jan. 24, 2005; PCT/US05/25860, filed Jul. 20, 2005, the disclosures of which are hereby incorporated by reference) and said embryoid bodies are plated in standard tissue culture vessels in the presence of DMEM media supplemented with 10% fetal bovine serum and pooled members of the FGF family FGF-2, FGF-8, FGF-15, FGF-17 at concentrations at the ED50 for each factor as is well known in the art to obtain a heterogeneous population of cells enriched in neuronal cell types. The media of said cultures is collected after 24 hours and the cultures are refed. The collected media is pooled, filtered through a 0.2 micron sterile filter and stored at 4° C. as conditioned medium. After a total of 10 days of differentiation, the differentiated cells are plated at limiting dilution, photographed to document the cell number in each well as well as the differentiated state of the cell, and fed the conditioned medium with biweekly refeeding, and cultured for two weeks in low ambient oxygen (5%), then microscopically analyzed for colony formation. The observed single cell-derived colonies, or clones, can then be expanded, cryopreserved, quality controlled, and their pattern of gene expression tested using gene expression arrays as is well known in the art.

In this example, colonies with a pattern of gene expression consistent with that of neuronal cells are useful in research and cell transplantation.

### Example 10

### Identification of Differentiated Tissues and Cells from Genetically Modified hES Cell Lines for Therapeutic Purposes

Master libraries of differentiated tissues and cell types from hES cells modified to prevent or reduce the severity of rejection by the host immune system may be ultimately used for therapeutic purposes. For example, dopaminergic neurons may be used to treat patients suffering from Parkinson's disease.

In this example, hES cells derived from 0 negative donors are first modified by gene targeting to delete the Major histocompatibility group loci HLA-A, HLA-B and HLA-D.

The same strategy for characterizing master libraries of differentiated hES cells is used to characterize cells that have been derived by directed differentiation. In this example, growth and analysis of dopaminergenic neurons are performed similar to Zeng et al., Stem Cells 22: 925-940 (2004). In brief, high throughput characterization of differentiated cells is performed by visually characterizing cell morphology and by microarray analysis of RNA transcripts to identify expression signatures specific for differentiated cells and tissues. Expression signatures by microarray analysis from differentiated cells and tissues are compared to existing microarray, SAGE, MPSS, and EST databases (Gene Expression Atlas, Affymetrix human Genechip U95A, http://expression.gnf.org; SAGEmap, http://www.ncbi.nlm.nih.gov/SAGE/; TissueInfo, http://icb.mssm.edu/crthissueinfowebservice.xml; UniGene, http://www.ncbi.nlm.nih.gov/UniGene/) to determine the cell or tissue type. Further additional characterization of differentiated cells and tissues may include immunocytochemistry for specific cell surface antigens, production of specific cell products, and 2D PAGE.

Growth of hESCs. Briefly, hESCs are maintained on inactivated mouse embryonic fibroblast (MEF) feeder cells in Dulbecco's modified Eagle's medium/Ham's F12 (DMEM/F12, 1:1) supplemented with 15% fetal bovine serum (FBS), 5% knockout serum replacement (KSR), 2 mM nonessential amino acids, 2 mM L-glutamine, 50 μg/ml Penn-Strep (Invitrogen, Carlsbad, Calif., http://www.invitrogen.com), 0.1 mM β-mercaptoethanol (Specialty Media, Phillipsburg, N.J., http://www.specialtymedia.com), and 4 ng/ml basic fibroblast growth factor (bFGF; Sigma, St. Louis, http://www.sigmaaldrich.com). Cells are passaged by incubation in Cell Dissociation Buffer (Invitrogen), dissociated, and then seeded at approximately 20,000 cells/cm². Under such culture condition, the ES cells are passaged every 4-5 days.

ECM components are applied to the culture substrate either to promote the generation of a heterogeneous mixture of differentiated cell types (candidate cultures) and/or for the propagation step. Many ECM components include: Gelatin, or Collagens V, VI, VII, VIII, IX, X, XI, XII, XIII, XIV, XV, XVI, XVII, XVIII and XIX.

Gelatin or specific collagens I-IX may be used to coat the culture substrate as follows. For short-term cultures of two days or less, the collagen solution is simply applied to the substrate and allowed to dry. The collagen solution is diluted 1:20 with 30% ethanol, spread over surface of sterile glass coverslip, and dried in a tissue culture hood. For long-term cultures or greater than two days, such as when culturing cell in the propagation step from a single cell or a small colony (oligoclonal propagation), the substrate can be first coated with polylysine or polyornithine. In this case, polylysine or polyornithine (MW or 30,000-70,000) at 0.1-1 mg/ml in 0.15 M borate buffer (pH 8.3) is filter sterilized and spread over the culture substrate. The covered substrate is incubated 2-24 hours at room temperature. The solution is then aspirated, washed three times with sterile water, and gelatin or specific collagens in solution (100 ug/ml in water) are added and incubated 4-16 hours. The solution is then aspirated, rinsed once with the medium to be used, and then seeded with cells in the medium used.

An alternative technique for long-term cultures generates a double layered collagen coating. The collagen solution as described above is spread on the substrate. This solution is immediately neutralized for 2 minutes with ammonium hydroxide vapors by placing the substrate in a covered dish containing filter paper wet with concentrated ammonium hydroxide. This will cause the collagen to gel. The substrate is then rinsed twice with sterile water and a thin film of the same solution is gently over the surface of the gelled collagen and air dried. The double layered collagen substrate is then used the same day for cell culture.

A polylysine-coated culture substrate can also be used as follows. A 0.01% solution of 150,000-300,000 molecular weight poly-D-lysine (Sigma P4832) is added to the culture vessel at about 0.5 mL per 25 cm² of surface area, incubated at 37° C. for 2-24 hours, removed, the substrate is rinsed twice with DPBS, and used immediately, or stored at 4° C.

Fibronectin may also be applied to the culture substrate. Fibronectin is an extracellular matrix constituent used for the culture of endothelial cells, fibroblasts, neurons and CHO cells. Briefly, stock solutions of fibronectin can be prepared by dissolving 1 mg/ml fibronectin in PBS, which is then filter sterilized and frozen in aliquots. The stock solution is diluted to 50-100 μg/ml in basal medium or PBS. Then, enough solution is added to pool over the surface of sterile glass coverslip. The coverslips can be incubated for 30-45 minutes at room temperature. The fibronectin solution is then aspirated to remove the excess fibronectin solution and the coverslips are then rinsed with media or PBS. Immediately thereafter, either cell suspension or growth media is added to prevent the fibronectin coating from drying.

Alternatively, laminin may be applied to the culture substrate. Laminin is an extracellular matrix constituent used for the culture of neurons, epithelial cells, leukocytes, myoblasts and CHO cells. Briefly, stock solutions of laminin can be prepared by dissolving 1 mg/ml laminin in PBS, which is then filter sterilized and frozen in aliquots. The stock solution is diluted to 10-100 μg/ml in basal medium or PBS. Then, enough solution is added to pool over the surface of sterile glass coverslip. The coverslips can be incubated for several hours at room temperature. The laminin solution is then aspirated to remove the excess laminin solution and the coverslips are then rinsed with media or PBS. Immediately thereafter, either cell suspension or growth media is added to prevent the fibronectin coating from drying. Furthermore, coating the glass coverslip first with polylysine or polyornithine followed by coating with laminin may increase the concentration of laminin applied using this method.

Neural Differentiation. Neural differentiation of ES cells is induced by the mouse stromal cell line PA6 as described by Kawasaki et al., Neuron, 28:31-40 (2000), with some modifications. hESCs are cultured to form colonies on PA6 feeder cells in Glasgow minimum essential media (Invitrogen) supplemented with 10% KSR (Invitrogen), 1 mM pyruvate (Sigma), 0.1 mM nonessential amino acids, and 0.1 mM b-mercaptoethanol. ES cell colonies are grown at a density of 1,000 colonies per 3-cm dish. The medium is changed on days 4 and 6 and every day thereafter.

Immunocytochemistry. Expression of stem cell and neuronal markers is examined by immunocytochemistry, and staining procedures are as described previously Zeng et al., Stem Cells, 21:647-653 (2003). Briefly, the ES cells are fixed with 4% paraformaldehyde and permeabilized with 0.1% Triton X-100. After blocking, the cells are incubated with primary antibody. The primary antibodies and the dilution used are as follows: Nestin and bromodeoxyurindine (BrdU [BD Pharmingen, San Diego, Calif., http://www.bdscience.com], 1:500 and 1:200); neural cell adhesion molecule (NCAM), synapsin, synaptophysin, and dopamine beta hydroxylase (DBH [Chemicon, Temecula, Ca, http://www.chemicon.com], 1:200, 1:20, 1:100, and 1:200); and neuron-specific class III beta tubulin (TuJ1) and tyrosine hydroxylase (TH [Sigma], 1:2000 and 1:2000, respectively). Localization of antigens is visualized by using respective secondary antibodies (Alexa fluor 594 or 488; Molecular Probes, Eugene, Oreg., http://www.probes.com).

Reverse Transcription-Polymerase Chain Reaction. Total RNA is extracted from undifferentiated or differentiated cells using RNA STAT-60 (Tel-Test Inc., Friendswood, Tex.). cDNA is synthesized using a reverse transcription kit (RETROscript, Ambion, Austin, Tex.) with 100 ng total RNA in a 20-μl reaction according to the manufacturer's recommendations. RNase H 1 μl (Invitrogen) is added to each tube and incubated for 20 minutes at 37° C. before proceeding to the reverse transcription-polymerase chain reaction (RTPCR) analysis. For each PCR reaction, 0.5-μl cDNA template is used in a 50-μl reaction volume with the RedTaq DNA polymerase (Sigma). The cycling parameters are as follows: 94° C., 1 minute; 55° C., 1 minute; 72° C., 1 minute for 30 cycles. The PCR cycle is preceded by an initial denaturation of 3 minutes at 94° C. and followed by a final extension of 10 minutes at 72° C. Real-time PCR is used to quantify the levels of mRNA expression of Nurr1. PCR reactions are carried out using an Opticon instrument (MJ Research, Waltham, Mass.) and SYBR Green reagents (Roche Molecular Biochemicals, Indianapolis) according to the manufacturer's instructions. The content of Nurr1 is normalized to the content of the housekeeping gene cyclophilin. Standard curves are generated by cloning amplified products, using human cDNA as a template, into the PCR4 vector (TOPO TA cloning kit [Invitrogen]). The purified fragment solution is measured in a spectrophotometer, and the molecular number is calculated. Plasmid solutions are then used to generate serial dilutions. PCR analyses are conducted in triplicate for each sample. The primer pairs used for real-time PCR analyses are sequence verified. The acquisition temperature for each primer pair is 3° C. below the determined melting point for the PCR product being analyzed.

Detection of Dopamine. hES cells are cultured on a PA6 cell layer for 3 weeks and rinsed twice with Hanks' balanced salt solution (HBSS). To induce depolarization, 56 mM KCl is added into the cells for 15 minutes. The medium is then collected and stabilized with 0.1 mM EDTA and analyzed for dopamine and DOPAC. Dopamine and DOPAC levels are measured using an HPLC coupled to an ESA Coulochem II Detector (Model 5200, ESA, Inc., Chelmsford, Mass.) with a dual-electrode microdialysis cell. Data are analyzed using an ESA data station (Model 501). Samples (20 μl) are injected by an autosampler (CMA 280) into a C-18 reverse-phase column (3 μm; particle size, 3μ 150 mm; Analytical MD-150 [ESA, Inc.]). The mobile phase for dopamine separation consists of 75 mM NaH2PO4, 1.5 mM 1-octanesulfonic acid-sodium salt, 10 μM EDTA, and 7% acetonitrile (pH 3.0, adjusted with H3PO4). Dopamine and DOPAC are quantified using the reducing (−250 mV) and oxidizing electrodes (350 mV), respectively, and then calculated as nanomolar concentration. The limit of detection is approximately 0.3 μg per injection.

Focused Microarray Analysis. The nonradioactive GEArray™ Q series cDNA expression array filters for human stem cell genes pathway genes and mouse cytokine genes (Hs601 and MM-003N, SuperArray Inc, http://superarray.com) (Luo et al., Stem Cells, 21:575-587 (2003)) are used according to the manufacturer's protocol. The biotin 2′-deoxyuridine-5′-triphosphate (dUTP)-labeled cDNA probes are specifically generated in the presence of a designed set of gene-specific primers using total RNA (4 μg per filter) and 200 U MMLV reverse transcriptase (Promega, San Luis Obispo, Calif., http://www.promega.com). The array filters are hybridized with biotin-labeled probes at 60° C. for 17 hours. After that, the filters are washed twice with 2× standard saline citrate (SSC)/1% SDS and then twice with 0.1×SSC/1% SDS at 60° C. for 15 minutes each. Chemiluminescent detection steps are performed by incubation of the filters with alkaline phosphatase-conjugated streptavidin and CDP-Star substrate. Array membranes are exposed to X-ray film. Quantification of gene expression on the array is performed with ScionImage software. cDNA microarray experiments are done twice with new filters and RNA isolated at different times. Results from the focused array are independently confirmed, and the array itself is validated (Wang et al., Exp Neurol 136:98-106 (1995)).

Of the 266 genes represented by the array, 50 genes are expressed in the induced neurons but not detected in undifferentiated cells. These include 14 markers for stem and differentiated cells, 22 growth factors and receptors, adhesion molecules, and cytokines, six extracellular matrix molecules, and eight others. In particular, Sox1, Map2, TrkC, and NT3 are expressed at higher levels in the differentiated cultures, which is consistent with results obtained by RT-PCR.

The expression of markers for dopaminergic neurons, as well as other neuronal markers, in hESC-derived differentiated cells is examined by immunocytochemistry, RT-PCR, and microarrays. The markers associated with the mature dopaminergic neuronal phenotype: TH, DAT, AADC, GTPCH, PCD, DHPR, and VMAT2 are expressed. The growth factor receptors TrkA, TrkB, TrkC, GFRA1, GFRA2, GFRA3, p75R, and c-ret and the Shh receptors Ptch and Smo are also present. Transcription factors Nurr1, Ptx3, Lmx1b, and Sox-1 associated with dopaminergic and neuronal differentiation are expressed by the PA6 cell-induced cells. Nurr1 is detectable in both undifferentiated hESCs and PA6-differentiated cells, but quantitative RT-PCR verified that a threefold increase in expression was associated with differentiation. DBH was not expressed in the TH-positive cells by immunostaining or RTPCR, and little or no NA was released by KCl stimulation, supporting the conclusion that PA6-induced hESC-differentiated cells are dopaminergic rather than noradrenergic. In addition to dopaminergic markers, cholinergic (ChAT and VAChT) and glutamatergic (GAC and KGA) markers were detected in the induced neurons, indicating the potential for generation of multiple neuronal types by this method. On the other hand, undifferentiated ES cell markers (hTERT, Oct3/4, Dppa5, and UTF-1) are not expressed in the differentiated cultures, indicating that undifferentiated hESCs do not persist in hESC cultures differentiated on PA6 cells.

### Example 11

Any pluripotent stem cells, such as ES cell lines and embryos, ICMs or blastomeres directly differentiated without making lines, may be used as the source of generating the cells of the present invention. Direct differentiation refers, for example, to methods of making downstream stem cells from an embryo without making ES cells (see U.S. patent publication no. 20050265976, published Dec. 1, 2005, and international patent publication no. WO0129206, published Apr. 26, 2001, the disclosures of which are hereby incorporated by reference). The resulting cells are eEmbryo-derived” (“ED”) cells, meaning cells made from embryos by directly differentiating them in vitro without making ES cell lines.

In this example; hES cells are derived from a single blastomere of a cryopreserved embryo wherein the original embryo is cryopreserved again and the blastomere is used to generate a female O-hES cell line with the HLA knockout. These hES cell colonies are differentiated using in situ colony differentiation by culturing them in conditions that induce differentiation without removing the colonies from their culture vessel, such as conditions that occur in the differentiation matrix shown in FIG. 1, in this example, condition #456 which is removal of LIF and the addition of 10% FBS. After various periods of time (1-100 days) in this example, 6 days, the cells are trypsinized and plated at limiting dilution such that most wells have a single cell. The wells are photodocumented to demonstrate a single cell is resident and that it does not have the morphological parameters of an ES cell. The plates are incubated in low ambient oxygen (5%) for ten days and microscopically analyzed for the presence of cell colonies. Colonies are photographed, trypsinized and passaged in the same media and characterized by gene expression as described below. Based on the type of tissue, the cells are lapelled by lentivirus carrying GFP or other markers such as beta galactosidase and injected into the corresponding tissue in an immunocompromised mouse to test engraftment.

### Example 12

Human blastocyst ICMs are isolated by immunosurgery and ICMs are plated in conditions to promote the direct differentiation of the ICM. Direct differentiation refers, for example, to methods of making downstream stem cells from an embryo without making ES cells (see U.S. patent publication no. 20050265976, published Dec. 1, 2005, and international patent publication no. WO0129206, published Apr. 26, 2001, the disclosures of which are hereby incorporated by reference). The resulting cells are “embryo-derived” (“ED”) cells, meaning cells made from embryos by directly differentiating them in vitro without making ES cell lines. In this example, ICM-derived cells are from a nuclear transfer embryo that is female O- and HLA knockout. They are differentiated by culturing them in conditions that induce ICM in situ differentiation, such as conditions that occur in the differentiation matrix shown in FIG. 1, in this example, condition #456 which is removal of LIF and the addition of 10% FBS. After various periods of time (1-100 days) in this example, 6 days, the cells are trypsinized and plated at limiting dilution such that most wells have a single cell. The wells are photodocumented to demonstrate a single cell is resident and that it does not have the morphological parameters of an ES cell. The plates are incubated in low ambient oxygen (5%) for ten days and microscopically analyzed for the presence of cell colonies. Colonies are photographed, trypsinized and passaged in the same media and characterized by gene expression as described below. Based on the type of tissue, the cells are lapelled by lentivirus carrying GFP or other markers such as beta galactosidase and injected into the corresponding tissue in an immunocompromised mouse to test engraftment.

### Example 13

Colonies from the hES cell line ACT3 were differentiated using in situ colony differentiation by culturing the cells in conditions that induce differentiation without removing the colonies from their initial culture vessel, such as conditions that occur in the differentiation matrix shown in FIG. 1. In this example, the condition used was #456 which is removal of LIF-containing medium and the addition of DMEM medium containing 10% FBS. At various intervals of time (5, 7, and 9 days of exposure to differentiation medium), the cells are trypsinized, and plated onto 15 cm gelatinized plates and cultured for an additional 20 days to further induce differentiation into a heterogeneous mixture of early embryonic cell types as the final candidate culture. Therefore, in this example, the cells were differentiated into candidate cultures of heterogeneous differentiated cell types using two sequential differentiation-inducing conditions, one being condition #456 (removal of LIF and the addition of 10% FBS), and the second being #339 (grown in media without LIF with 10% FBS and grown on gelatin ECM).

The cells appeared largely fibroblastic, though heterogeneous in appearance and were then trypsinized and counted with a Coulter counter, and a volume containing 2,500 cells, 5,000 cells and 25,000 cells was introduced into gelatinized 15 cm tissue culture plates containing DMEM medium supplemented with 10% FBS, rocked twice counterclockwise, twice clockwise, twice vertically, twice horizontally to disperse the cells and subsequently incubated in 5% ambient oxygen undisturbed for two weeks.

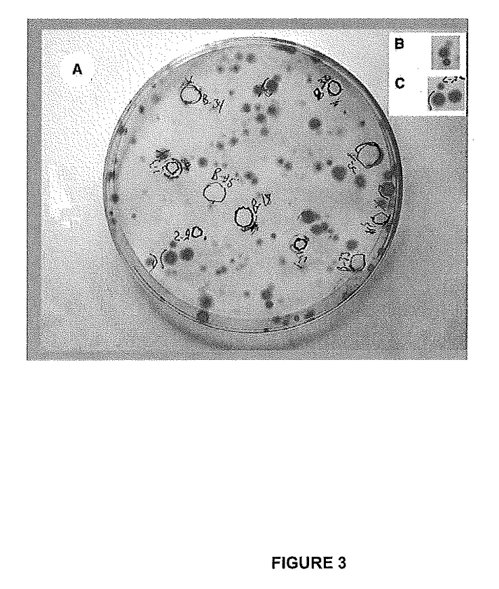

Clonal colonies were identified by phase contrast microscopy and those that are uniformly circular and well separated from surrounding colonies were marked for removal using cloning cylinders as is well known in the art. The dish of colonies at day 9 of in situ differentiation followed by 20 days of in vitro differentiation on gelatin and plated at 2,500 cell per dish was stained with crystal violet solution for 10 minutes, rinsed with water and is shown in FIG. 3.

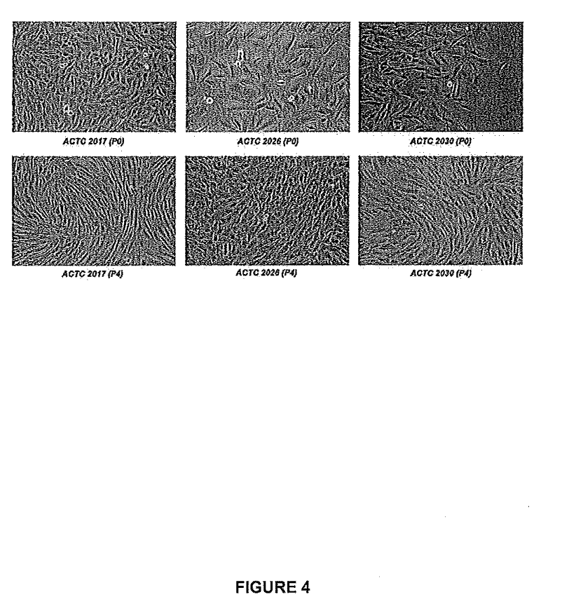

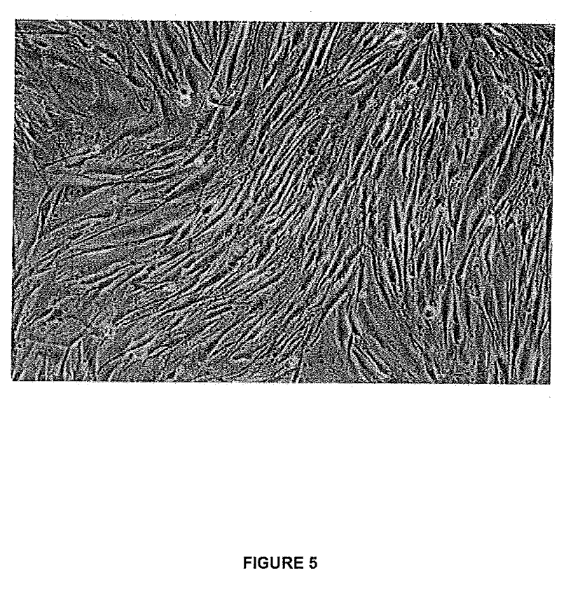

The trypsinized cells from within 61 cloning cylinders (P0) were then replated into gelatinized 24 well plates and incubated. Of 61 colonies isolated, 45 clonal populations became confluent in the 24 well plates (P1) and were then trypsinized and plated in 12 well gelatinized plates (P2). Of these, 44 wells became confluent and these were in turn trypsinized and plated in 6 well gelatinized plates (P3). Of these, 40 became confluent and were transferred to two six well gelatinized plates (P4). Of these, 34 became confluent and were trypsinized and plated in a 100 mm gelatinized tissue culture dish (P5). Of these, 16 became confluent and were trypsinized and transferred to gelatinized T75 flasks (P6). Representative phase contrast photographs of cells in the original clonal colony (P0) and after the fourth passage (P4) are shown in FIG. 4.

The cell cultures tested displayed a normal human karyotype. RNA was harvested from the cells in order to characterize the cell strains and the nature of their differentiated state. Other aliquots of cells were plated onto glass coverslips for immunocytochemical characterization of their differentiated state using antibodies to antigens such as are listed in Table V.

### Example 14

Colonies from the hES cell line ACT3 were differentiated using in situ colony differentiation by culturing the cells in conditions that induce differentiation without removing the colonies from their initial culture vessel, such as conditions that occur in the differentiation matrix shown in FIG. 1. In this example, the condition used was #456, which is removal of LIF-containing medium and the addition of DMEM medium containing 10% FBS. The cells were differentiated for 7 days by exposure to differentiation medium, and viable, day 7 differentiated cells were determined via trypan blue exclusion method.

Day 7 differentiated cells were used in this experiment because the dermal progenitor clone B-2 (ACTC #59) was isolated from these differentiated cells. The cells were cultured in either DMEM with various concentrations of FBS or in specialized media.

For the culturing of cells in DMEM media with 3 different FBS concentrations, approximately 1,000 day 7 differentiated cells were plated in 15 cm gelatin-coated tissue culture plates containing DMEM media with either 5% FBS, 10% FBS or 20% FBS. Each media tested was carried out in replicates of 5 dishes per data point.

For the culturing of cells in specialized media, approximately 2,500 and 10,000 of day 7 differentiated cells were plated in 15-cm gelatin-coated tissue culture plates containing any one of the following cell selection/growth media in Table VI:

TABLE VI
Cell Selection and Growth Media

| | Media | Manufacturer | Catalog Number | Addition |
|---|---|---|---|---|
| 1 | Airway | PromoCell | C-21260 | Manufacturer |
| | Epithelial | | | Supplement |
| | Growth | | | |
| | Medium | | | |
| 2 | Epi-Life | Cascade | M-EPIcf/PRF-500 | LSGS (Low |
| | (LSGS) | | | Serum Growth |
| | Medium. | | | Supplement) |
| 3 | Neurobasal | Gibco | 12348-017 | B27 |
| | Medium - B27 | | | |
| 4 | Neurobasal | Gibco | 12348-017 | N2 |
| | Medium - N2 | | | |
| 5 | HepatoZyme- | Gibco | 17705-021 | None |
| | SFM | | | |
| 6 | Epi-Life | Cascade | M-EPIcf/PRF-500 | HKGS (Human |
| | (HKGS) | | | Keratinocyte |
| | Medium. | | | Growth |
| | | | | Supplement) |
| 7 | Endothelial | PromoCell | C-22221 | Manufacturer |
| | Cell Growth | | | Supplement |
| | Medium | | | |
| 8 | Endothelial | Gibco | 11111-044 | Epithelial |
| | Cell SFM | | | Growth |
| | | | | Factor, Basic |
| | | | | Fibroblast |
| | | | | Growth Factor |
| 9 | Skeletal | PromoCell | C-23260 | Manufacturer |
| | Muscle Growth | | | Supplement |
| | Medium | | | |
| 10 | Smooth Muscle | PromoCell | C-22262 | Manufacturer |
| | Basal Medium | | | Supplement |
| 11 | MesenCult | Stem Cell | 05041 | Manufacturer |
| | | Technologies | | Supplement |
| 12 | Melanocyte | PromoCell | C-24010 | Manufacturer |
| | Growth | | | Supplement |
| | Medium | | | |

The cell selection/growth media may preferentially select and sustain growth of particular cell phenotypes for which they were designed. Each media tested was carried out with one plate of each cell concentration. The day 7 differentiated cells cultured in either the DMEM/FBS or cell selection/growth media were allowed to grow for 7-10 days to form colonies, the colonies cloned and plated in 24-well gelatin-coated plates containing the same medium in which they were grown. The individual colonies are expanded to obtain a stock of cells and the cell line stocks are cryopreserved. During the clonal expansion protocol, samples of the cell lines are taken for gene expression and immunophenotype analysis.

### Example 15

Cells from human ES (hES) cell line H-9 passage #48 were plated in a standard 6 well tissue culture plate on a feeder layer of mouse embryonic fibroblasts and allowed to grow for 9 days to confluence. The hES cell growth medium was replaced by 6 differentiation media as shown in Table VII, and the hES cells were allowed to differentiate for 3 days.

TABLE VII
Differentiation Media

| hES | | | | |
|---|---|---|---|---|
| Cell | | | | |
| Well | Differentiation | Manu- | Catalog | |
| Number | Medium Addition | facturer | Number | Addition |
| 1 | Airway Eiphelial | PromoCell | C-21260 | Manufacturer |
| | Growth Medium | | | Supplement |
| 2 | Neurobasal | Gibco | 12348- | B-27 |
| | Medium - B27 | | 017 | |
| 3 | Epi-Life | Cascade | M- | LSGS (Low |
| | Medium - | | EPIcf/PRF- | Serum Growth |
| | LSGS | | 500 | Supplement) |
| 4 | Endothelial Cell | PromoCell | C-22221 | Manufacturer |
| | Growth Medium | | | Supplement |
| 5 | Skeletal Muscle | PromoCell | C-23260 | Manufacturer |
| | Cell Growth | | | Supplement |
| | Medium | | | |
| 6 | DMEM + | Hyclone | SH302285- | 10% fetal |
| | 10% FBS | | 03 | bovine serum |

The cells were trypsinized using 0.05% trypsin and transferred to Corning 6-well, ultra low attachment tissue culture plates containing 12 embryoid body media as shown in Table VIII, and allowed to form embryoid bodies.

TABLE VIII
Embryoid Body Media

| | | Embryoid | | | |
|---|---|---|---|---|---|
| hES Cell | | Body Well | | | |
| Well | Differentiation | (Ultra Low | | | |
| (Original | Medium (Original | Attachment | Embyoid Body | | Catalog |
| Plate) | Plate) | Plate) | Media | Manufacturer | Number |
| Well 1 | Airway Eiphelial | 1 | Airway | PromoCell | C-21260 |
| | Medium | | Eiphelial | | |
| | | | Growth Medium | | |
| | | 2 | Epi-Life | Cascade | M- |
| | | | (LSGS) Medium | | EPIcf/PRF- |
| | | | | | 500 |
| Well 2 | Neurobasal | 3 | Neurobasal | Gibco | 12348-017 |
| | Medium - B27 | | Medium - B27 | | |
| | | 4 | Neurobasal | Gibco | 12348-017 |
| | | | Medium - N2 | | |
| Well 3 | Epi-Life (LSGS) | 5 | HepatoZyme- | Gibco | 17705-021 |
| | Medium. | | SFM | | |
| | | 6 | Epi-Life | Cascade | M- |
| | | | (HKGS) Medium | | EPIcf/PRF- |
| | | | | | 500 |
| Well 4 | Endothelial Cell | 7 | Endothelial | PromoCell | C-22221 |
| | Medium | | Cell Growth | | |
| | | | Medium | | |
| | | 8 | Endothelial | Gibco | 11111-044 |
| | | | Cell SFM | | |
| Well 5 | Skeletal Muscle | 9 | Skeletal | PromoCell | C-23260 |
| | Cell Medium | | Muscle Cell | | |
| | | | Growth Medium | | |
| | | 10 | Smooth Muscle | PromoCell | C-22262 |
| | | | Basal Medium | | |
| Well 6 | DMEM + 10% FBS | 11 | DMEM + 20% | Hyclone | SH302285- |
| | | | FBS | | 03 |
| | | 12 | Melanocyte | PromoCell | C-24010 |
| | | | Growth Media | | |

One well of differentiated hES cells were divided equally between 2 wells containing 2 different media and allowed to form embryoid bodies. For example, well number 1 of the original 6 well plate in which the hES cells were allowed to differentiate in Airway Eiphelial Medium for 3 days and then were trypsinized and half the cells are placed in a well of an ultra low attachment plate containing the same Airway Eiphelial Medium and the other half of the cells transferred to a second well of the ultra low attachment plate containing Epi-Life LSGS Medium.

The embryoid bodies were allowed to differentiate for 7-10 days, collected, washed in phosphate buffered saline, dissociated into single cells with trypsin (0.25% trypsin) and the differentiated cells plated out in extra cellular matrix coated 15 cm plates (see Table IX). The differentiated cells are allowed to proliferate for 7-20 days and the resulting colonies are cloned and plated in 24 well plates containing the same medium and extra cellular matrix from which they were derived. The cloned colonies are expanded to obtain a stock of cells and the cell line stocks are cryopreserved.

TABLE IX
Extracellular Matrix & Growth Medium

| | | Extra |
|---|---|---|
| | | Cellular |
| 15 cm Plate | Selection & Growth Media | Matrix |
| 1 | Airway Eiphelial Growth Medium | Gelatin |
| 2 | Epi-Life (LSGS) Medium. | Collagen IV |
| 3 | Neurobasal Medium - B27 | Poly-lysine - |
| | | BioCoat |
| 4 | Neurobasal Medium - N2 | Poly-lysine - |
| | | BioCoat |
| 5 | HepatoZyme-SFM | Collagen IV |
| 6 | Epi-Life (HKGS) Medium. | Collagen IV |
| 7 | Endothelial Cell Growth Medium | Gelatin |
| 8 | Endothelial Cell SFM | Gelatin |
| 9 | Skeletal Muscle Cell Growth Medium | Gelatin |
| 10 | Smooth Muscle Basal Medium | Gelatin |
| 11 | DMEM + 20% FBS | Gelatin |
| 12 | Melanocyte Growth Medium | Gelatin |

During the clonal expansion protocol, samples of the cell lines are taken for gene expression and immunophenotype analysis.

### Example 16

Colonies from the hES cell line ACTS were differentiated using in situ colony differentiation by culturing them in conditions that induce differentiation without removing the colonies from their culture vessel, such as conditions that occur in the differentiation matrix shown in FIG. 1. In this example, the condition used was #456, which is removal of LIF and the addition of 10% FBS. At intervals of 5, 7, and 9 days after the colonies had begun to differentiate, the cells were trypsinized, and 25,000 cells were plated onto 15 cm gelatinized plates and cultured for an additional 20 days to further induce differentiation into a heterogeneous mixture of early embryonic cell types as the final candidate culture. These cells were then cryopreserved using DMSO as is well known in the art. The cells were subsequently thawed, cultured for two days on different ECMs (gelatin, plasma fibronectin, poly-D-lysine, and tenscin-C) and in chemically-defined, serum-free medium (Lifeline Fibrolife Medium LM-0001). The cells were then trypsinized and counted with a Coulter counter, and a volume containing 5,000 cells in the case of day 5, and 1,000 cells in the case of days 7 and were introduced into 150 mm tissue culture dishes with the same medium and array of ECMs and subsequently incubated in 5% ambient oxygen undisturbed for two weeks with the exception of feeding after one week. Colonies are then identified by phase contrast microscopy, isolated, expanded, and characterized as described above in Example 13.

### Example 17

### Single Cell-Derived Cell Lines of Series 1 and 2

To derive the cells of the two series designated Series 1 and 2, colonies from the hES cell line ACTS were routinely cultured in hES medium (KO-DMEM, 1× nonessential amino acids, 1× Glutamax-1, 55 uM beta-mercaptoethanol, 10% Serum Replacement, 10% Plasmanate, 10 ng/ml LIF, 4 ng/ml bFGF) and passaged by trypsinization. hES cells were plated at 500-10,000 cells per 15 cm dish. Three days after passaging, the cells were differentiated using colony in situ differentiation by the removal of LIF-containing medium and the addition of DMEM medium containing 10% FBS (Table I, conditions #456 and #1103). After various periods of time (5, 7, and 9 days of exposure to differentiation medium), the cells were trypsinized and plated onto 15 cm plates at low density of approximately 1,000 cells per cm² coated with the extracellular matrix protein Type I collagen (gelatin) (Table I, condition #339), and cultured for an additional 20 days to further induce differentiation in the same conditions in which they will subsequently be clonally expanded (the enrichment step). In the case of the Series 1 cells, the cells were then trypsinized and counted with a Coulter counter, and the cells were plated at increasing dilutions with a volume containing 2,500 cells, 5,000 cells and 25,000 cells introduced into the 15 cm tissue culture plates and subsequently incubated in 5% ambient oxygen (Table I, condition #449) undisturbed for two weeks.

Clonal colonies were identified by phase contrast microscopy and those that are uniformly circular and well separated from surrounding colonies were marked for removal using cloning cylinders.

The trypsinized cells from within each cloning cylinder were then replated into collagen coated 24 well plates and incubated. Of 61 colonies isolated, 54 grew at a relatively rapid rate of approximately one doubling a day. The cells were karyotyped and determined to be normal human. Colonies were serially grown in gelatinized 24 well, 12 well, 6 well tissue culture plates, T25, T75, T150 flasks, and in some cases to 2 liter Roller Bottles (850 cm² surface area) before freezing and storing in liquid nitrogen. Of 61 colonies isolated from the cells of Series 1, 43 grew at a relatively rapid rate of approximately one doubling a day. Of these colonies, 19 cultures propagated to 150 cm² flasks and were then cryopreserved using 10% tissue grade DMSO in ethanol chambers and were assigned ACTC numbers (see Table XII). All of those cell lines described in the present invention assigned ACTC numbers displayed the capacity for propagation in vitro. Those cell lines not given an ACTC number displayed a capacity for propagation from one cell to approximately 5×10⁵ cells but may or may not show the capacity for long-term propagation in vitro beyond that point. The cells were karyotyped and determined to be normal human. Cell morphologies and cell growth were monitored by phase contrast microscopy and recorded by photomicroscopy. Cells were cultured in 6 well tissue culture plates or 6 cm tissue culture Petri dishes prior to freezing to harvest mRNA for gene expression analysis using the Illumina human sentra-6 platform. The cell lines isolated are shown in the table below.

In the case of Series 2, Day 9 cells that had been cryopreserved were thawed, cultured for five days in 10% FBS supplemented DMEM medium, then trypsinized, counted, and 2,000 cells were plated onto gelatinized 15 cm dishes in 10% FBS supplemented DMEM medium but with 0%, 10%, 20%, 30% or 50% of the same medium that was previously conditioned for 48 hours on the same starting population of heterogeneous cells, clarified by centrifugation at 10,000×g, and stored at 4 deg C. until use. The cell clones were then isolated as described in the case of series 1, and the lines isolated in the various conditioned media are shown in the table below.

| Series 1 Exp. | | | Series 2 Exp. | | |
|---|---|---|---|---|---|
| Line | ACTC | | Line | ACTC | |
| Name | No. | Medium | Name | No. | Medium |
| 1 | | DMEM 10% Fetal | CM0-1 | | DMEM |
| 2 | | Bovine Serum | CM0-2 | 77 | 10% Fetal |
| 3 | | | CM0-3 | 73 | Bovine Serum |
| 4 | | | CM0-4 | | |
| 5 | | | CM0-5 | 74 | |
| 6 | | | CM10-1 | | |
| B-1 | | | CM10-2 | | |
| B-2 | 51 | | CM10-3 | | |
| B-3 | 55 | | CM10-4 | | |
| B-4 | 66 | | CM20-1 | | |
| B-5 | | | CM20-2 | | |
| B-6 | 56 | | CM20-3 | | |
| B-7 | 53 | | CM20-4 | 79 | |
| B-9 | | | CM20-5 | | |
| B-10 | | | CM30-1 | | |
| B-11 | 58 | | CM30-2 | 78 | |
| B-12 | 65 | | CM30-3 | | |
| B-13 | | | CM30-4 | | |
| B-14 | 67 | | CM30-5 | | |
| B-15 | 71 | | CM50-1 | | |
| B-16 | 59 | | CM50-2 | 76 | |
| B-17 | 54 | | CM50-3 | | |
| B-18 | | | CM50-4 | 72 | |
| B-19 | | | CM50-5 | 75 | |

| | |
|---|---|
| B-20 | TOTAL COLONIES |
| B-21 | SERIES 2 = 24 |

| | |
|---|---|
| B-22 | |
| B-23 | |
| B-24 | |
| B-25 | 57 |
| B-26 | 50 |
| B-27 | |
| B-28 | 60 |
| B-29 | 52 |
| B-30 | 61 |
| B-31 | |
| B-32 | |
| B-33 | |
| B-34 | |
| B-35 | |
| 2-1 | 63 |
| 2-2 | 62 |
| 2-3 | 70 |
| 2-4 | |
| 4-1 | |
| 4-2 | 69 |
| 4-3 | |
| 4-4 | |
| 5-1 | |
| 5-2 | |
| 5-3 | |
| 5-4 | 68 |
| 5-5 | |
| 6-1 | 64 |

TOTAL COLONIES
SERIES 1 = 54

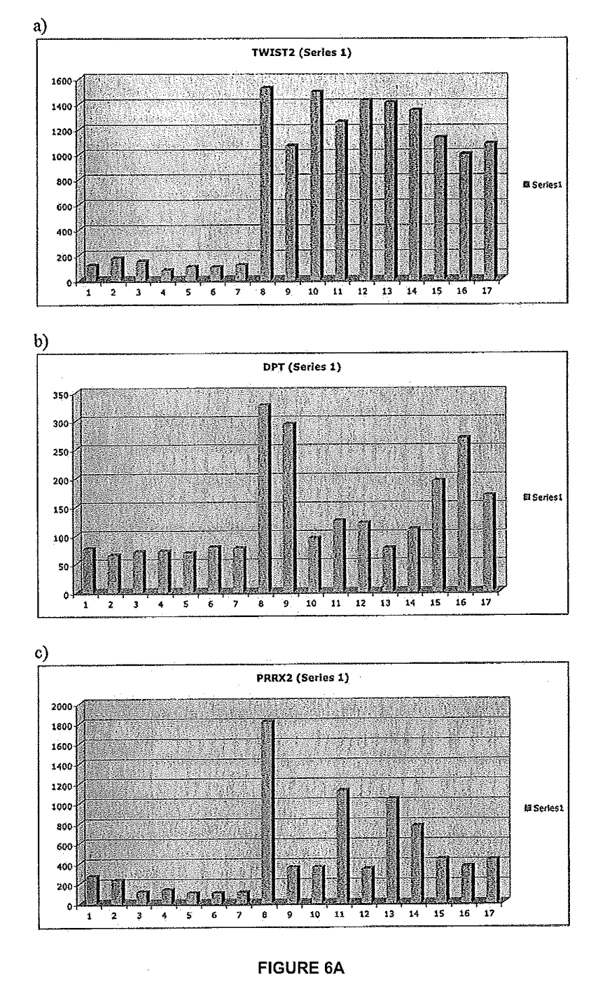

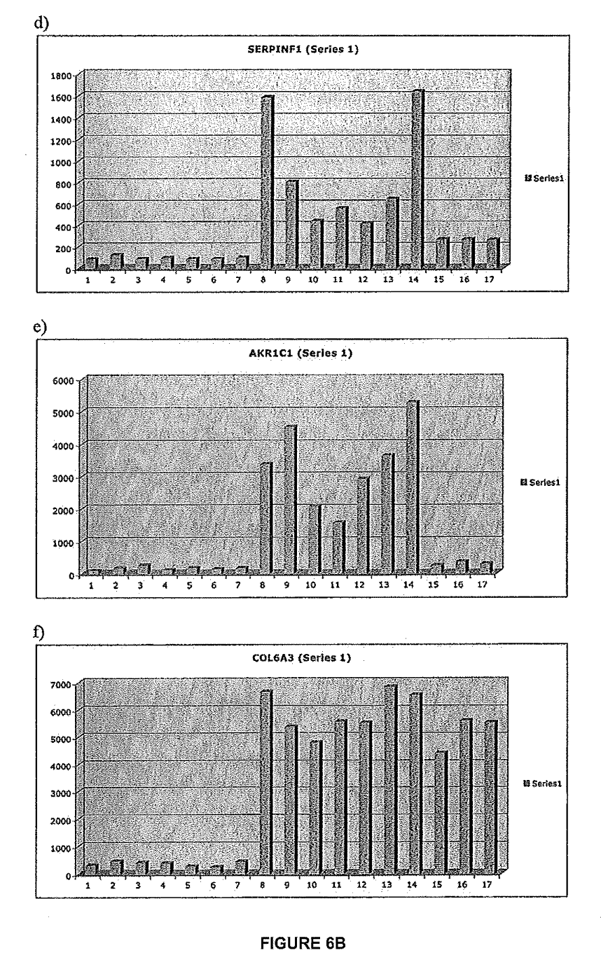

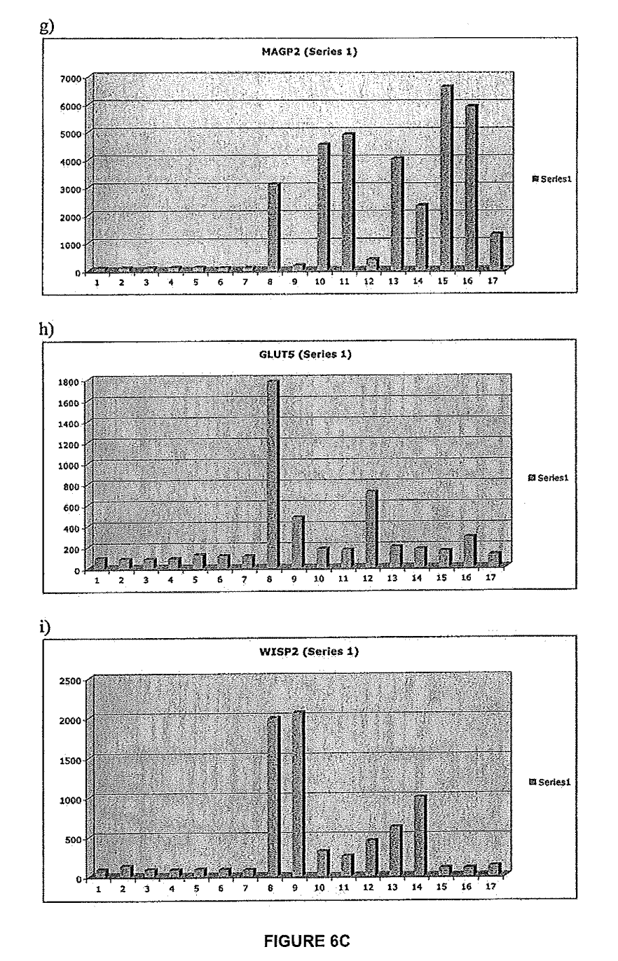

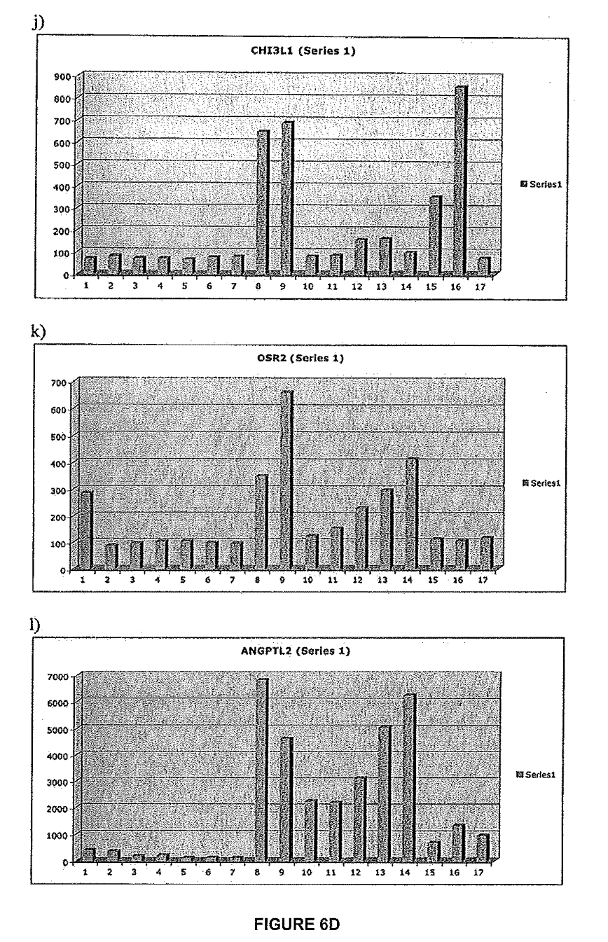

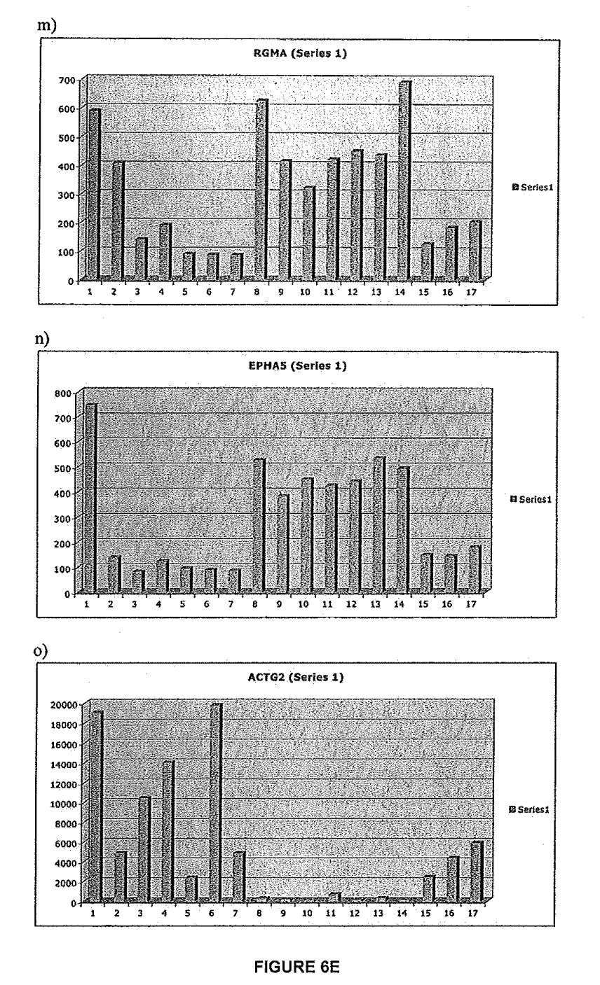

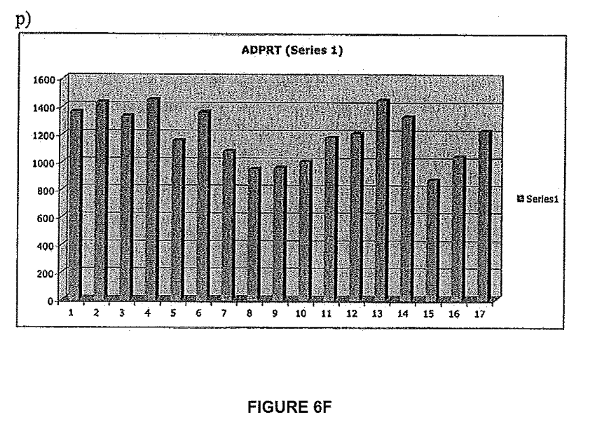

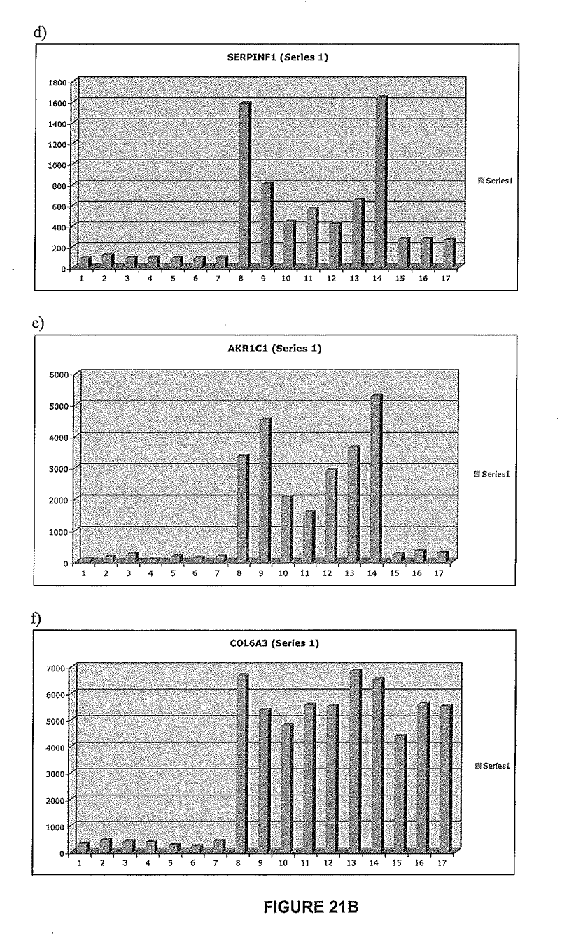

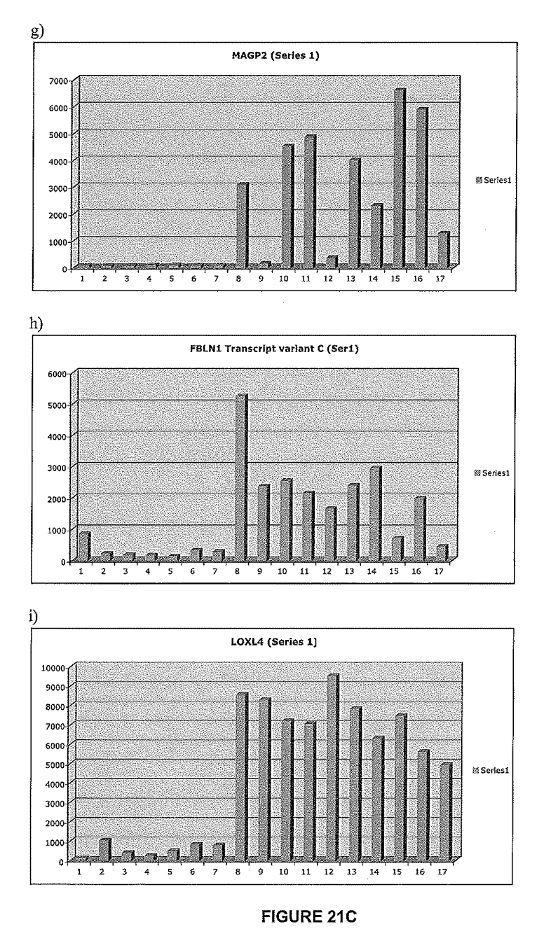

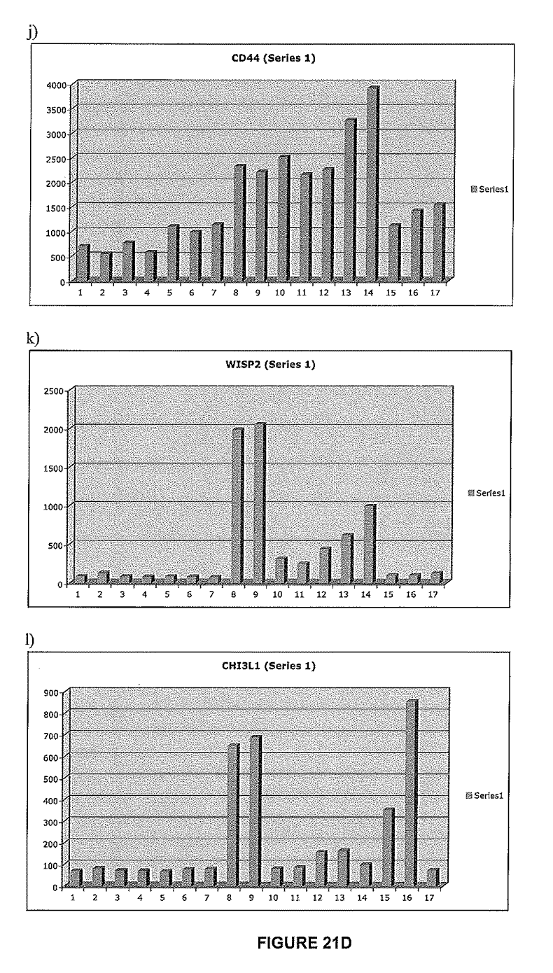

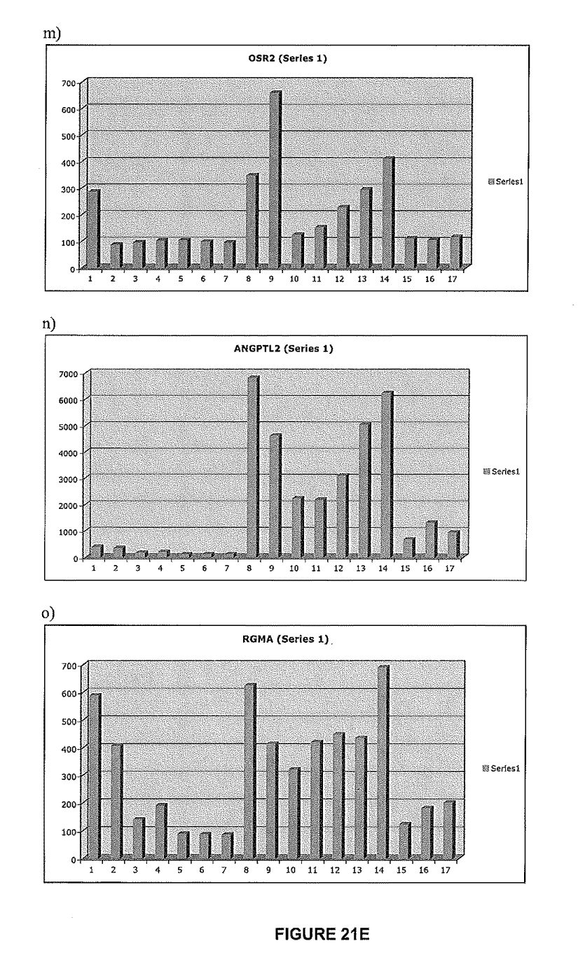

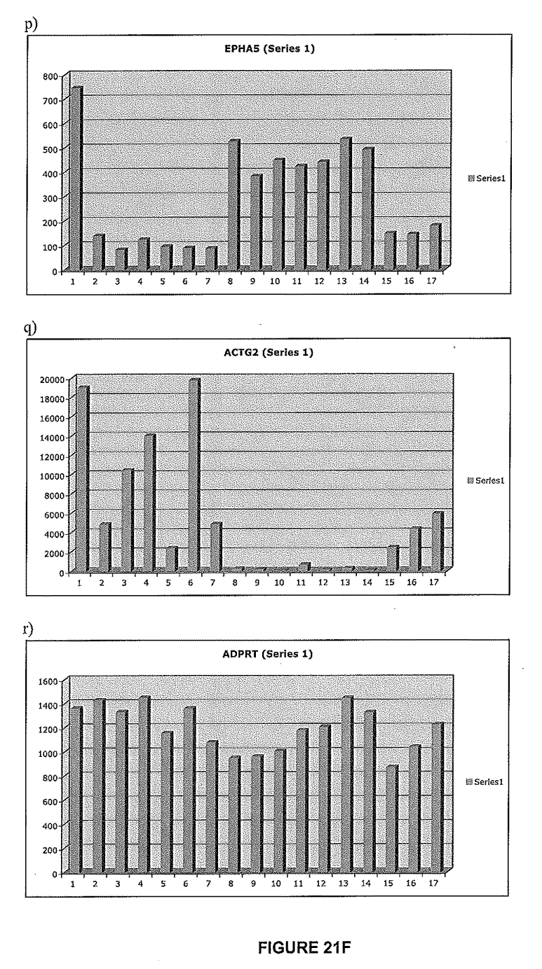

Of the first 17 colonies for which gene expression analysis was performed, clone 8 (132 or ACTC51) of Series 1 displayed a pattern of gene expression consistent with dermal fibroblast progenitors with its expression of dermo-1 (TWIST2), dermatopontin (DPT), PRRX2 (which is a marker of fetal scarless wound repair (J Invest Dermatol 111(1):57-63 1998)), PEDF (SERPINF1), AKR1C1, collagen VI/alpha 3 (COL6A3), microfibril-associated glycoprotein 2 (MAGP2), which is a component of elastin-associated microfibrils, a component associated with elastogenesis Fibulin-1 (FBLN1). In developing prenatal skin, the MAGP2 protein is detected in the deep dermis and around hair follicles. The expression of MAGP2 has been reported to be up to six-fold higher in the prenatal state than postnatal and its expression precedes elastin synthesis in development (Gibson et al., J. Histochem. Cytochem. 46(8): 871-886 (1998)), GLUTS, WISP2, CHI3L1, Odd-Skipped Related 2 (OSR2), angiopoietin-like 2 (ANGPTL2), RGMA, EPHA5, the receptor for hyaluronic acid which promotes scarless wound repair (CD44), and a relative lack of the smooth muscle actins of a myofibroblast such as Actin Gamma 2 (ACTG2) (see FIGS. 6 and 21).

In developing prenatal skin, the MAGP2 protein is detected in the deep dermis and around hair follicles. The expression of MAGP2 has been reported to be up to six-fold higher in the prenatal state than postnatal and its expression precedes elastin synthesis in development (Gibson et al., 1998).

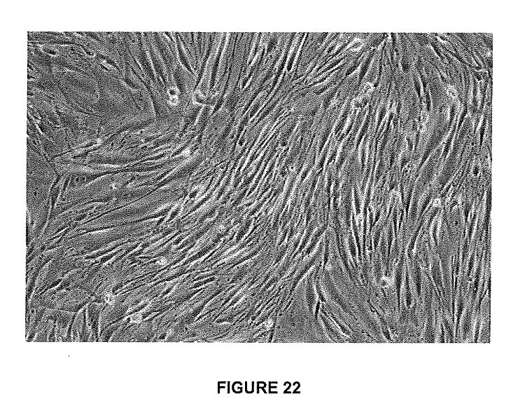

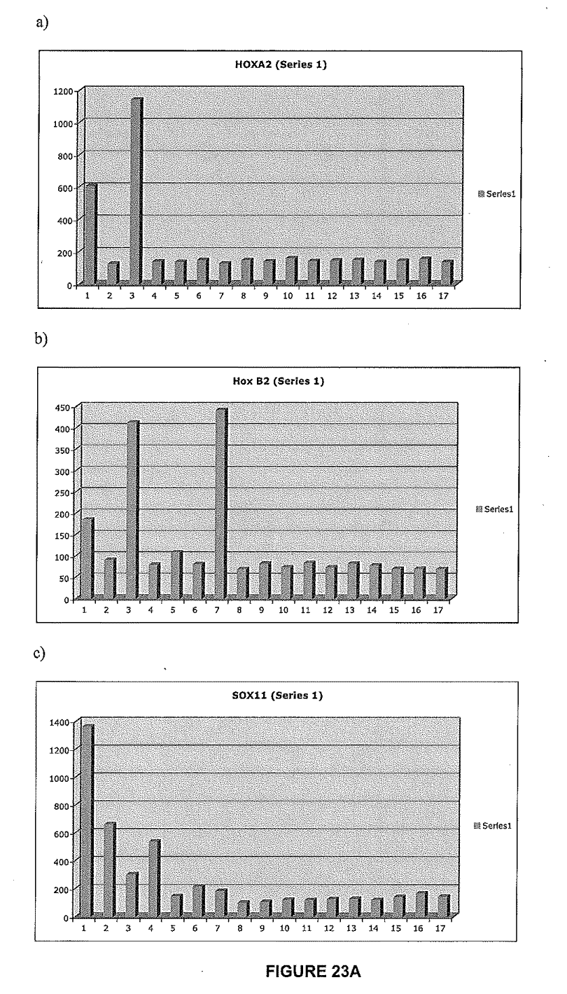

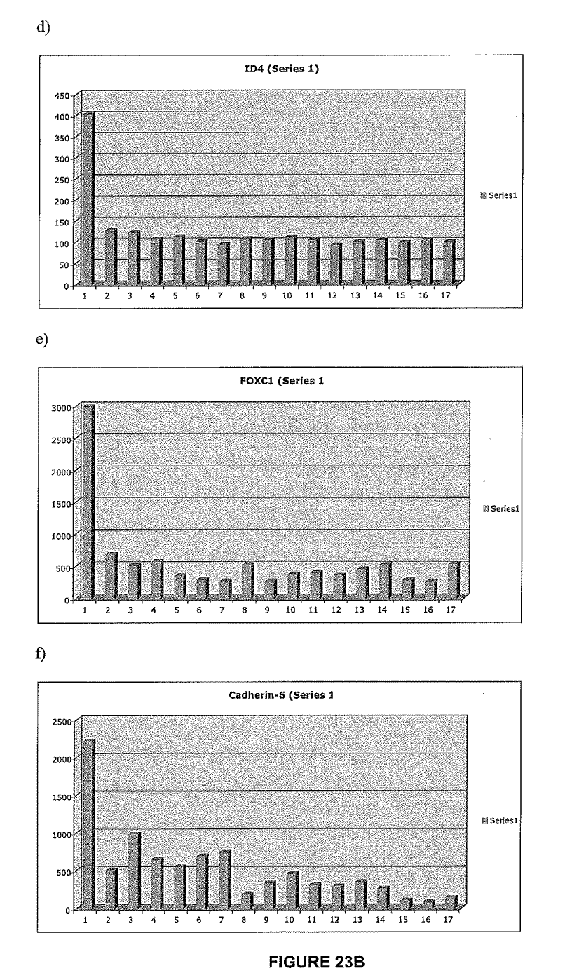

Markers that uniquely identify dermal progenitors from this region of the developing dermis include the positive expression of TWIST2, DPT, PRRX2, MAGP2, and WISP2 at levels comparable to ADPRT as shown in FIGS. 6 and 21, and the relative lack of expression of ACTG2 in relation to ADPRT as shown. A phase contrast photograph of the dermal fibroblast progenitors is shown in FIG. 22. All levels of gene expression were compared to the internal reference expression of the housekeeping ADPRT gene.

The relatively abundant expression of EPHA5 and RGMA in these dermal progenitors promote neuronal outgrowth and innervation of the forming tissues, are therefore useful in regenerating skin while promoting the innervation of the skin graft with sensory neurons and is an example of genes not expressed at comparable levels postnatally. The relatively abundant expression of angiopoietin-like2 (ANGPTL2) is another example of dermal cells with a prenatal pattern of gene expression, able to promote vascularization.

### Example 18

According to the methods described in Example 17, a number of other genes that are normally expressed more broadly in the embryo than postnatally were observed to be expressed by the clonogenic cells derived in this invention. The following markers were uniquely expressed in our other cell lines that are normally expressed more broadly in the embryo than postnatally:

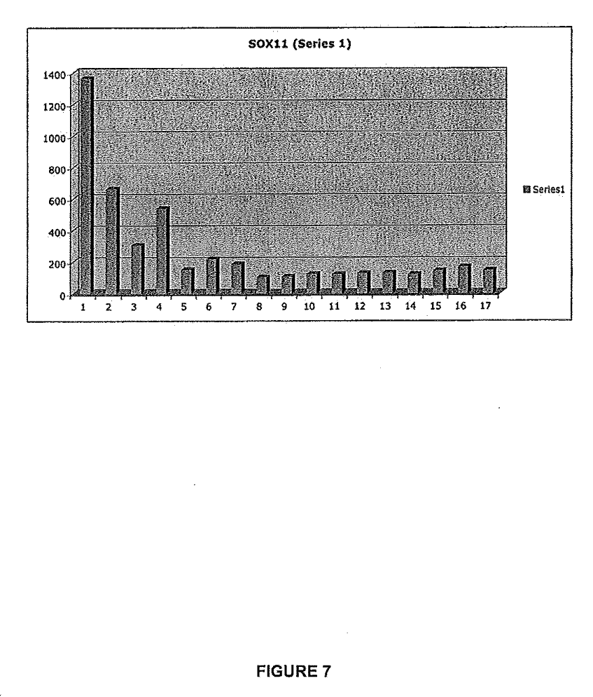

The SOX11 gene was expressed by the cells derived from clone 1 (B30 or ACTC61) of Series 1 (see FIG. 7 and Example 17). SOX11 is a gene which is largely expressed only in the CNS in adults, but has also been reported to be expressed in other places in the embryo, including the neural crest, mammary anlagen, ear fold, nose, and limb buds.

Some complement components, such as C3, MASP1, carboxypeptidases such as CPE and CPZ, like Furin activate prohormones and other proteins in early embryogenesis, but in the later fetal and adult stages of development, these complement components and other embryonic proteases are largely used only for the complement cascade or digestion. CPE (carboxypeptidase E) is a prohormone convertase like furin and is primarily CNS, neural crest, and expressed in the embryonic ribs, ganglia, in first branchial arch, embryonic heart, cartilage, primordial cells of cephalic bones, developing vertebral bodies, dorsal surface of tongue, and olfactory epithelium.

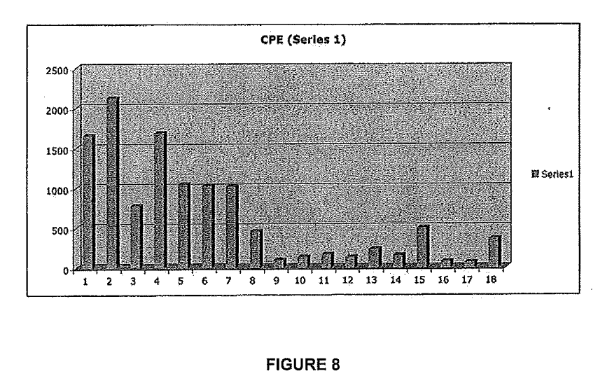

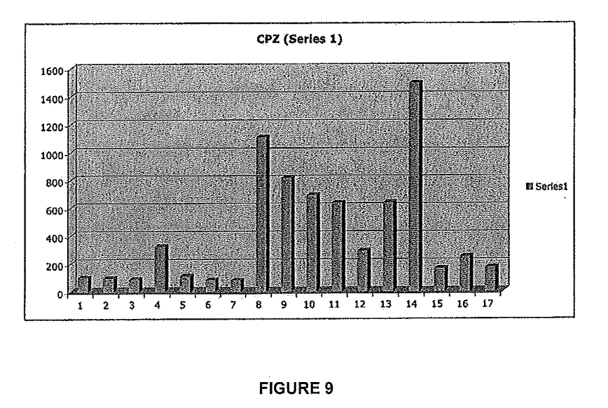

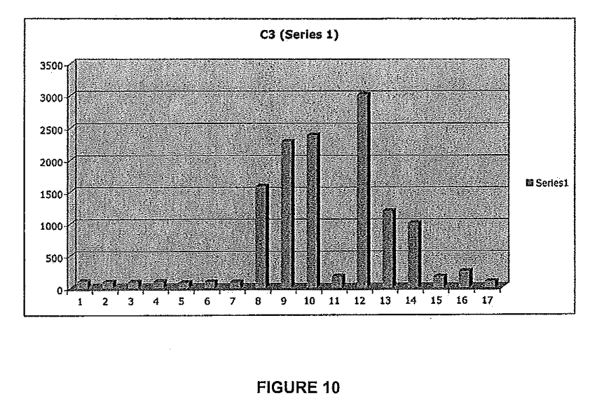

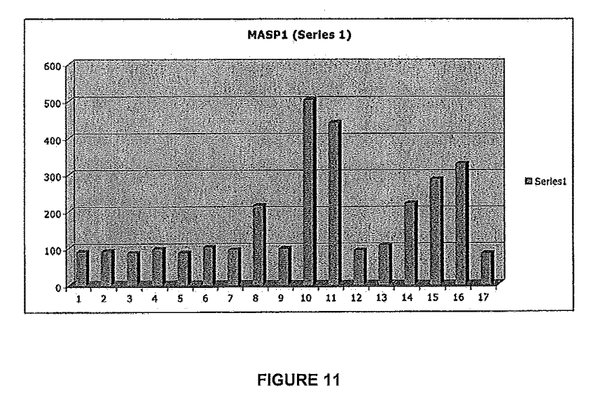

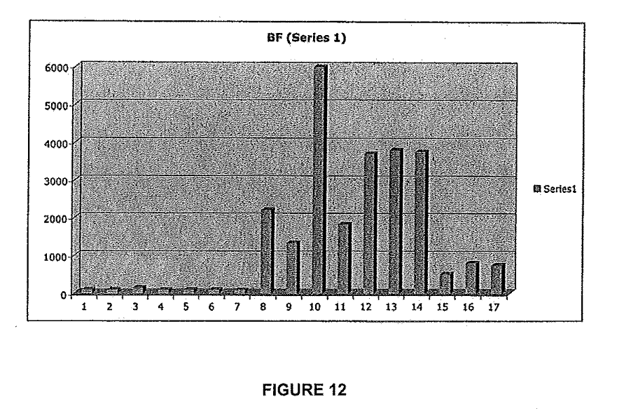

Examples of cells displaying this embryonic pattern of complement proteases and thereby capable of inducing tissue generation and regeneration were observed. The CPE gene was expressed by the cells derived from clones 1 (B30 or ACTC61), 2 (B17 or ACTC54), 4 (B6 or ACTC56), 5 (4-1), 6 (4-3) and 7 (B-10) of Series 1 (see FIG. 8). The CPZ gene was expressed by the cells derived from clones 8 (b2 or ACTC51), 9 (B7 or ACTC53), 10 (B25 or ACTC57), 11 (B11 or ACTC58), 13 (B26 or ACTC50) and 14 (6-1 or ACTC64) of Series 1 (see FIG. 9). The C3 gene was expressed by the cells derived from clones 8 (B2 or ACTC51), 9 (B7 or ACTC53), 10 (B25 or ACTC57) and 12 (B3 or ACTC55) of Series 1 (see FIG. 10). The MASP1 gene was expressed by the cells derived from clones 8 (B2 or ACTC51), 10 (B25 or ACTC57), 11 (B11 or ACTC58), 14 (6-1 or ACTC64), 15 (2-2 or ACTC62) and 16 (2-1 or ACTC63) of Series 1 (see FIG. 11). Finally, the BF gene was expressed by the cells derived from clones 10 (1325 or ACTC57), 12 (B3 or ACTC55), 13 (B26 or ACTC50) and 14 (6-1 or ACTC64) of Series 1 (see FIG. 12).

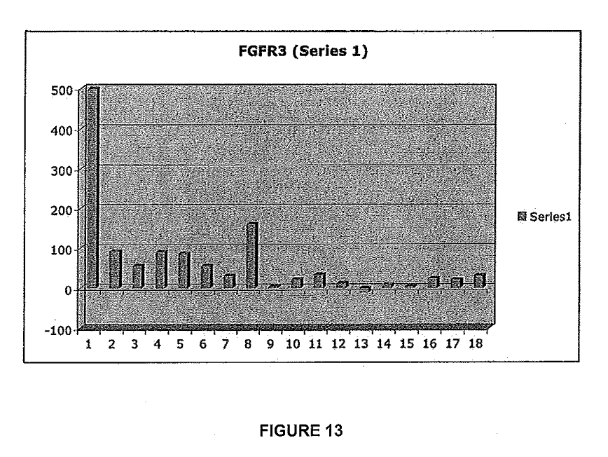

The FGFR3 (FGF Receptor 3) gene was expressed by the cells derived from clone 1 (b30 or ACTC61) of Series 1 (see FIG. 13). The FGFR3 (FGF Receptor 3) gene is expressed primarily in the CNS but also in other tissues during embryogenesis.

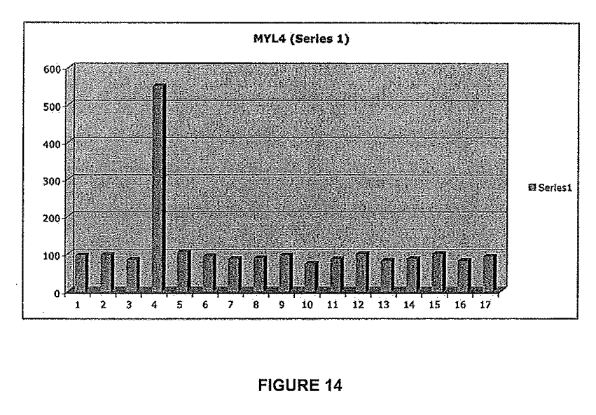

The MYL4 (myosin light chain 1) gene was also specifically expressed by the cells derived from clone 4 (B6 or ACTC56) of Series 1 (see FIG. 14). MYL4 is an atrial/fetal isoform of the protein, indicating a muscle precursor of the first branchial arch that may be useful in research and for regenerating muscles of the derivatives of the first branchial arch such as muscles of the mandible.

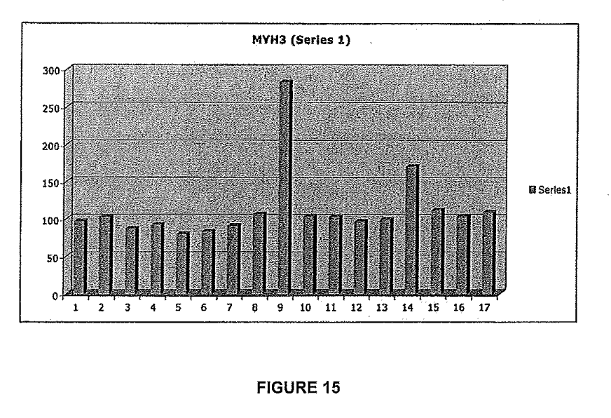

The MYH3 (myosin heavy chain polypeptide 3) gene was expressed by the cells derived from clone 9 (B7 or ACTC53) of Series 1 (see FIG. 15). Since the MYH2 gene is normally expressed in embryonic skeletal muscle, the overexpression of this gene by the cells derived from clone 9 suggests that these cells may be embryonic muscle precursor cells.

### Example 19

One of the important aspects of the clonogenic differentiated cell lines generated according to the methods of this invention is the observation that the original cell can be photo-documented not to have the morphology of an ES cell, and the resulting colony and subsequent cultures have vanishingly small likelihood of harboring undifferentiated ES cells. Since hES cells can only grow as colonies and as such, have unique and easily-recognized morphology as well as requiring special growth conditions, the likelihood for hES cells existing within the clonogenic differentiated cell lines is highly unlikely.

Since the characterization of cell formulations for therapy will require extensive documentation that the formulation does not include ES cells, the clonogenic differentiated cell lines with reduced or no contaminating ES cells can be used to determine the threshold concentrations of contaminating ES (or EC) cells tolerable in hES-based therapeutics.

A gradient of doses of hES cells (which lead to benign teratomas) and human EC (hEC) cells (EC being a malignant version of ES called teratocarcinoma cells) will be transplanted into SCID mice. The amount of hES and hEC cells will be transplanted at a gradient dose, with smaller and smaller doses of the ES and EC cells transplanted with the clonogenic differentiated cells generated according to the methods of this invention, until at the end of the gradient spectrum, only the clonogenic differentiated cells are being administered.

First, for the transplantation of hES, two SCID mice will be injected with 3×10⁶ hES cells (GFP-H1) in one leg quadricep muscle. The animals will be sacrificed after 60 days and histology will be performed on teratoma. The human cells can be identified by means of fluorescence and antibodies directed to human Class I HLA.

Second, for the transplantation of hES-derived clonogenic cells, two SCID mice will be transplanted with 3×10⁶ cells obtained from Example 13 or Example 17. The animals will then be sacrificed after 60 days and histology will be performed on teratoma, identifying human cells by means of fluorescence and antibody to human Class I HLA.

Finally, a gradient of doses of hES or hEC will be mixed with the clonogenic differentiated cells generated by the present invention at 0.01%, 0.1%, 1%, and 10% of the total cell number. The sensitivity of the assay to detect ES cells will be determined in the mass of tissue. Evidence of benign or malignant growth or metastasis will be determined.

Furthermore, the clonogenic differentiated cell lines can be mixed with GFP hES to allow visualization of the interaction of the cells with differentiating cells and tissues in a teratoma, thereby giving more insight into the nature and uses of the differentiated cell lines.

### Example 20

### Whole Body Imaging of Human Embryonic Stem Cells and Differentiated Progeny Cells in Mice

The locations and migration of human embryonic stem cells, and their differentiated progeny, in mouse tissues and cavities are identified by whole body imaging of mice injected with genetically modified hES cells, or their differentiated progeny, by technologies well know to those versed in the art. In this approach, cells that are genetically modified to express reporter genes are introduced into mice by injection directly into the target tissue, or introduced by intravenous or intraperitoneal injection. Cells may be genetically modified with a transgene encoding the Green Fluorescent protein (Yang, M., et al. (2000) Proc. Natl. Acad. Sci. USA, 97:1206-1211), or one of its derivatives, or modified with a transgene constructed from the Firefly (Photinus pyralis) luciferase gene (Fluc) (Sweeney, T. J. et al. (1999) Proc. Natl. Acad. Sci. USA, 96: 12044-12049), or with a transgene constructed from the Sea Pansey (Renilla reniformis) luciferase gene (Rkuc) (Bhaumik, S., and Ghambhir, S. S. (2002) Proc. Natl. Acad. Sci. USA, 99:377-382). The reporter transgenes may be constitutively expressed using a “house-keeping gene” promoter such that the reporter genes are expressed in many or all cells at a high level, or the reporter transgenes may be expressed using a tissue specific or developmental stage specific gene promoter such that only cells that have located into particular niches and developed into specific tissues or cell types may be visualized.

Creation of Luciferase or GFP Expressing Clonogenic Cell Lines. Human ES cells or their differentiated progeny are first genetically modified with expression vectors containing reporter genes encoding the Firefly luciferase gene (FLuc), Renilla luciferase gene (RLuc), or green fluorescence protein (GFP), or similar fluorescence proteins. These reporter gene vectors are available from commercial vendors as plasmid or retroviral vectors ready-for-use, or are engineered as proprietary expression vectors. There are several advantages to engineering proprietary reporter vectors for the applications described herein: tissue specific or developmental stage-specific promoters can be used to mark and identify specific classes or types of differentiated cells in vitro and in vivo; choice of plasmid or viral vector allows optimizing delivery of the reporter vector to cells; and construction of vectors with proprietary reporter genes not commercially available.

In this example, we describe the procedure for generating hES cells, or their differentiated progeny, including the dermal progenitor cells ACTC 59 (B2), containing the pFB-Luc retroviral vector (Stratagene, La Jolla, Calif.) stably integrated into the cellular genomic DNA. Luciferase levels and cell transduction efficiencies are determined by measuring luciferase activity in lysates of virus infected cells, by immunocytochemically staining cells for Luciferase expression, and by direct detection of luminescent cells in culture.

Transduction of Target Cells with a Viral Supernatant. This transduction is performed to demonstrate that cell lines are able to be transduced, that the viral supernatants are able to be transduced, and to assess the quality of the viral supernatants.

Day 1: Preparing for Transduction

1. For both NIH3T3 positive control cells and target cells, including the dermal progenitor cells ACTC 59 (B2), seed 6 wells using 6-well tissue culture plates with 1×105 cells per well. This seeding density may vary with the target cell line; ˜20% confluency at the time of infection is desirable.

2. Return the plates to the 37° C. incubator overnight.

Day 2: Transducing the Target Cells

Prior to thawing the viral supernatant, the area around the cap should be carefully inspected for any sign of leakage, and thoroughly wiped with 70% ethanol. Media should be prepared and aliquoted into prelabeled Falcon® 2054 polystyrene tubes prior to thawing the virus.

1. Quickly thaw the pFB-Luc supernatant (nominal titer approximately 2×10⁷/ml) by rapid agitation in a 37° C. H2O bath. Screw caps should be removed in the hood only, and any fluid around the outside lip of the tube or the inside surface of the cap should be carefully wiped with a tissue wetted with 70% ethanol, and the tissue should be disposed of in the hood. Thawed virus should be temporarily stored on ice if not used immediately.

2. Prepare a dilution series from 1:10 to 1:10⁴ in growth medium (2.0 ml dilution per tube in 2054 tubes) supplemented with DEAE-dextran at a final concentration of 10 μg/ml (1:1000 dilution of the 10 mg/ml DEAE-dextran stock). Add 0.8-1.0 ml undiluted supernatant to an additional tube, and supplement with DEAE-dextran to 10 μg/ml.

3. Remove the plates containing the target cells (NIH3T3 cells and target cells) from the incubator.

4. Remove and discard the medium from the wells. For tubes containing undiluted supernatant and for each dilution, add 1.0 ml per well to both the NIH3T3 and target cell. Add 1.0 ml media (no virus) to the sixth well for an uninfected control. The remaining supernatant should be aliquoted and refrozen at −80° C. It should be noted that the titer will drop, resulting in a loss of <50% of the remaining infectious particles with each subsequent freeze-thaw cycle.

5. Return the plates to the 37° C. incubator and incubate for 3 hours.

6. After the 3 hour incubation, add an additional 1.0 ml growth medium to each well.

7. Return the plates to the 37° C. incubator and allow 24-72 hours for analysis of expression of the luciferase protein by luciferase assay, immunocytochemistry, or direct visualization of luminescent cells.

Luciferase Assay. Transduction efficiencies of cells are determined by assaying lysates of virus infected cells for luciferase production. Luciferase may be assayed using commercially available kits. In this example, we describe measuring luciferase production using a Luciferase assay kit from Stratagene (La Jolla, Calif.).

Extracting Luciferase from Tissue Culture Cells. The cell lysis buffer is designed to extract luciferase from mammalian tissue culture cells that are transfected with the luciferase reporter gene. The inclusion of 1% Triton® X-100 in the cell lysis buffer allows the direct lysis of many types of tissue culture cells, such as HeLa cells and fibroblasts. The quantities of the reagents given in this protocol are optimized for a 35-mm tissue culture plate having ˜9.4 cm2 of surface area in each well. The volume of the cell lysis buffer may be adjusted for tissue culture plates of other sizes.

1. Being careful not to dislodge any of the cells, remove the media from the tissue culture plate wells and wash the cells twice with 1×PBS.

2. Using a Pasteur pipet, remove as much PBS as possible from each well.

3. Make 1× cell lysis buffer (25 mM Tris-phosphate (pH 7.8), 2 mM DTT, 2 mM 1,2-diaminocyclohexane-N,N,N′,N′ tetraacetic acid, 10% glycerol, 1% Triton® X-100) by adding 4 milliliters of dH2O per milliliter of the 5× cell lysis buffer. Equilibrate the lysis buffer to room temperature before use.

4. Cover the cells by adding approximately 200-500 μl of 1× cell lysis buffer to each well.

5. Incubate the plate at room temperature for 15 minutes, swirling occasionally.

6. Scrape the cells and buffer from each well into separate microcentrifuge tubes. Place the tubes on ice.

7. Vortex the microcentrifuge tubes for 10-15 seconds. Spin the tubes in a microcentrifuge at 12,000×g for 15 seconds at room temperature or 2 minutes at 4° C.

8. Transfer the supernatant from each tube to a new microcentrifuge tube.

9. Immediately assay the supernatant for luciferase activity according to the protocol provided below or store the supernatant at −80° C. for later use. It should be noted that each freeze-thaw cycle results in a significant loss of luciferase activity (as much as 50%).

Performing Luciferase Activity Assay. The following protocol is based on a single-tube luminometer. Luminometers capable of assaying multi-well plates (e.g., 96-well plates) and sophisticated computer software to process large numbers of samples are also commercially available. Although both scintillation counters and photographic film can be used to detect the light emission, they are not as sensitive.

1. Prepare the luciferase substrate-assay buffer mixture by adding all of the assay buffer (10 ml) to the vial containing the lyophilized luciferase substrate and mixing well.

2. Divide the luciferase substrate-assay buffer mixture into aliquots of an appropriate size to avoid multiple freeze-thaw cycles. The luciferase substrate-assay buffer mixture is best if used within one month when stored at −20° C. or within one year when stored at −70° C. Avoid unnecessary freeze-thaw cycles. Protect the luciferase substrate-assay buffer mixture from light.

3. Allow the luciferase substrate-assay buffer mixture to reach room temperature. Allow the supernatant from step 9 in Extracting Luciferase from Tissue Culture Cells to reach room temperature.

4. Add 100 μl of the luciferase substrate-assay buffer mixture to a polystyrene tube that fits in the luminometer (e.g., a 5-ml BD Falcon polystyrene round bottom tube).

5. Add 5-20 μl of supernatant to the tube, mix gently, and immediately put the tube into the luminometer.

6. Begin measuring the light produced from the reaction ˜8 seconds after adding the supernatant using an integration time of 5-30 seconds.

Immunocytochemistry for Cells Expressing Luciferase. An aliquot of viral transduced cells are cultured for 3 days after which cells were harvested and prepared on cytospin slides. Slides are stained with monoclonal antiluciferase antibody (Novus, Littleton, Colo.) 1:100 for 1 hour, followed by donkey polyclonal antibody to mouse IgG-FITC (Novas) 1:100 for 30 minutes. The slides are mounted with Vectashield medium with DAPI (4′,6-diamidino-2-phenylindole; Vector Laboratory, Burlingame, Calif.). Cultured nontransduced cells are used as negative controls.

Direct Imaging of Luciferase Expressing Cells. Optimal conditions for DNA delivery are identified by adding luciferin (0.5 mg/ml final; Molecular Probes) to the cell culture medium and light emission is used to confirm expression of the reporter gene. Cultures are screened by using an intensified charge-coupled device camera (C2400-32, Hamamatsu Photonics, Hamamatsu City, Japan). Colonies of cells expressing light are expanded for xenotransplantation into mice.

Xenotransplantation of Cells into Mice. Mice are anesthetized by i.p. injection of approximately 40 μl of a ketamine and xylazine (4:1) solutions and injected with approximately 3×10⁶ Luciferase expressing cells in 100 μl of PBS directly into the peritoneal cavity or injected via tail-vein. Injected mice are allowed to recover, maintained in a controlled environment and monitored weekly for 8 weeks to track the migration and final destination of Luciferase expressing cells using Xenogen IVIS Imaging System 3D Series bioluminescence imagers. Luciferase expressing ACTC59(B2) dermal progenitor cells are injected intradermally at doses of 1×10³, 1×10⁴, 1×10⁵, and 1×10⁶ cells in three animals over 4 injections per animal and engraftment and migration of the cells are tracked over three months using Xenogen IVIS Imaging System 3D Series bioluminescence imagers.

Whole Body Imaging of Luc-Marked Cells Injected in Mice. Imaging of mice containing cells expressing Flue reporter genes requires injection of mice with the cofactor Luciferin for light production and anesthetization prior to imaging. Mice are injected by an intraperitoneal route into the animal's lower left abdominal quadrant using 1 cc syringe fitted with a 25 gauge needle with a luciferin solution (15 mg/ml or 30 mg/kg, in PBS, dose of 150 mg/kg; D-Luciferin, Firefly, potassium salt, 1.0 g/vial, Xenogen Catalog #XR-1001) that is allowed to distribute in awake animals for about 5-15 minutes. The mice are placed into a clear plexiglass anesthesia box (2.5-3.5% isofluorane) that allows unimpeded visual monitoring of the animals; e.g. one can easily determine if the animals are breathing. The tube that supplies the anesthesia to the box is split so that the same concentration of anesthesia is plumbed to the anesthesia manifold located inside the imaging chamber. After the mice are fully anesthetized, they are transferred from the box to the nose cones attached to the manifold in the imaging chamber of a Xenogen IVIS Imaging System 3D Series imager, the door is closed, and the “Acquire” button (part of the Xenogen Living Image program) on the computer screen is activated. The imaging time is between one to five minutes per side (dorsal/ventral), depending on the experiment. When the mice are turned from dorsal to ventral (or vice versa), they can be visibly observed for any signs of distress or changes in vitality. The mice are again imaged (maximum five minutes), and the procedure is complete. The mice are returned to their cages where they awake quickly.

Alternatively, for mice containing cells expressing the RLuc reporter genes, an aqueous solution of the substrate coelenterazine (Biotium; 3.5 mg/kg) is injected via tail vein 10 minutes before imaging. The animals are then placed in a light-tight chamber, and a gray-scale body-surface reference image is collected with the chamber door slightly open. For this purpose, a low-light imaging system, comprised of an intensified charge-coupled device camera fitted with a 50-mm f1.2 Nikkor lens (Nikon) and a computer with image-analysis capabilities, is used. Subsequently, the door to the chamber is closed to exclude the room light that obscures the relatively dimmer luciferase bioluminescence. Photons emitted from luciferase within the animal and then transmitted through the tissue are collected and integrated for a period of 5 min. A pseudocolor image representing light intensity (blue least intense and red most intense) is generated on an Argus 20 image processor (Hamamatsu); images are transferred by using a plug-in module (Hamamatsu) to a computer (Macintosh 8100/100) running an image processing application (PHOTOSHOP, Adobe Systems, Mountain View, Calif.). Gray-scale reference images and pseudocolor images are superimposed by using the image-processing software, and annotations are added by using another graphics software package (CANVAS, version 5.0, Deneba, Miami, Fla.).

In whole body imaging approaches using GFP, and derivative, proteins, mice are anesthetized with pentobarbital (70 mg/kg body weight) placed in a warmed light box or directly on the microscope stage. A Leica fluorescence stereo microscope, model LZ12, equipped with a 50-W mercury lamp, is used for high-magnification imaging. Selective excitation of GFP is produced through a D425y60 band-pass filter and 470 DCXR dichroic mirror. Emitted fluorescence is collected through a long-pass filter GG475 (Chroma Technology, Brattleboro, Vt.) on a Hamamatsu C5810 3-chip cooled color charge-coupled device camera (Hamamatsu Photonics Systems, Bridgewater, N.J.). Images are processed for contrast and brightness and analyzed with the use of IMAGE PRO PLUS 3.1 software (Media Cybernetics, Silver Springs, Md.). Images of 1,024 3 724 pixels are captured directly on an IBM PC or continuously through video output on a high-resolution Sony VCR model SLV-R1000 (Sony, Tokyo). Imaging at lower magnification that visualizes the entire animal is carried out in a light box illuminated by blue light fiber optics (Lightools Research, Encinitas, Calif.) and imaged by using the thermoelectrically cooled color charge-coupled device camera, as described above.

### Example 21

### hES-Derived Smooth Muscle Progenitors

Colonies from the hES cell line ACT3 were differentiated using in situ colony differentiation by the removal of LIF-containing medium and the addition of DMEM medium containing 10% FBS. After various periods of time (5, 7, and 9 days of exposure to differentiation medium), the cells were trypsinized, and plated onto 15 cm plates coated with the extracellular matrix protein collagen, and cultured for an additional 20 days to further induce differentiation. The cells were then trypsinized and counted with a Coulter counter, and the cells were plated at increasing dilutions with a volume containing 2,500 cells, 5,000 cells and 25,000 cells introduced into the 15 cm tissue culture plates and subsequently incubated in 5% ambient oxygen undisturbed for two weeks.

Clonal colonies were identified by phase contrast microscopy and those that are uniformly circular and well separated from surrounding colonies were marked for removal using cloning cylinders. The trypsinized cells from within each cloning cylinder were then replated into collagen coated 24 well plates and incubated. Of 61 colonies isolated, 29 grew at a relatively rapid rate of approximately one doubling a day. The cells were karyotyped and determined to be normal human. A total genomic expression analysis using the Illumina system was performed on the cells.

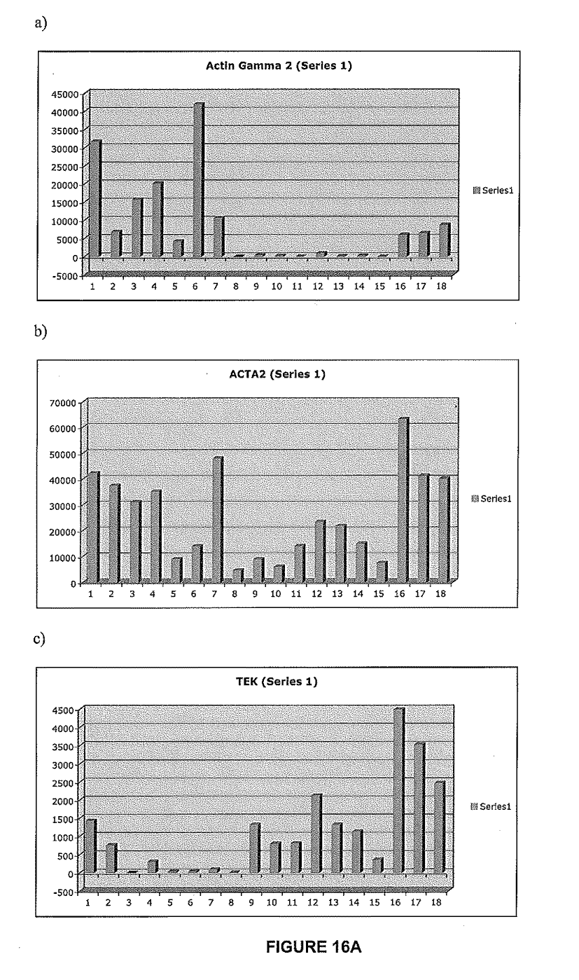

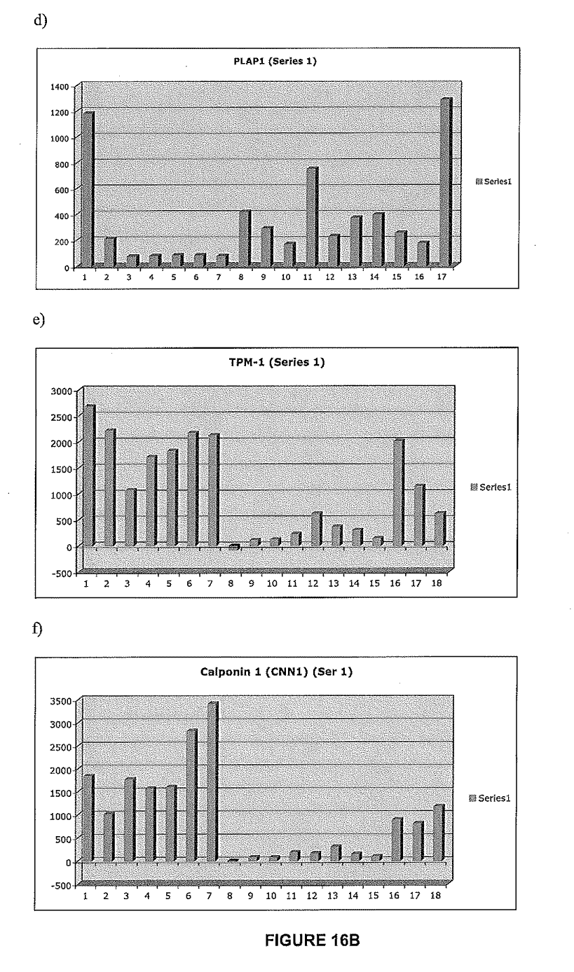

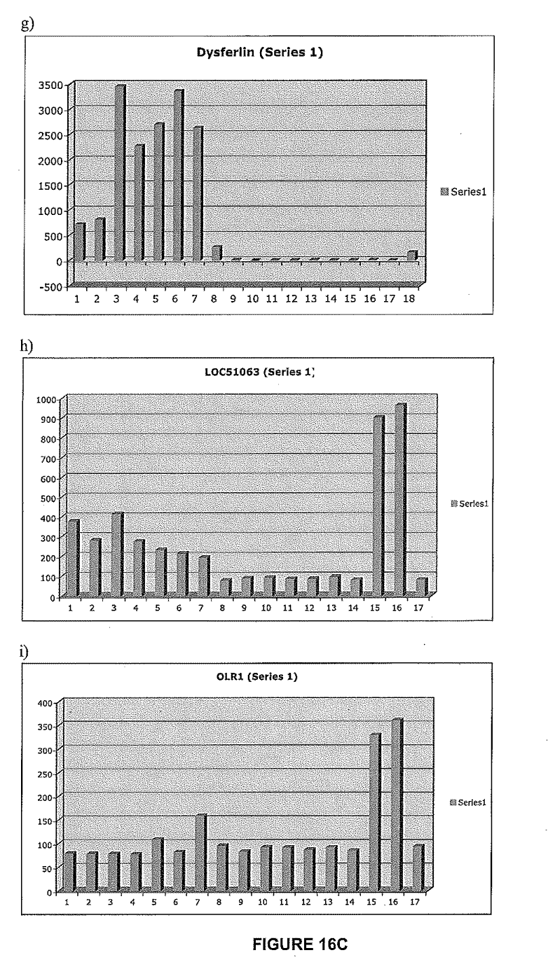

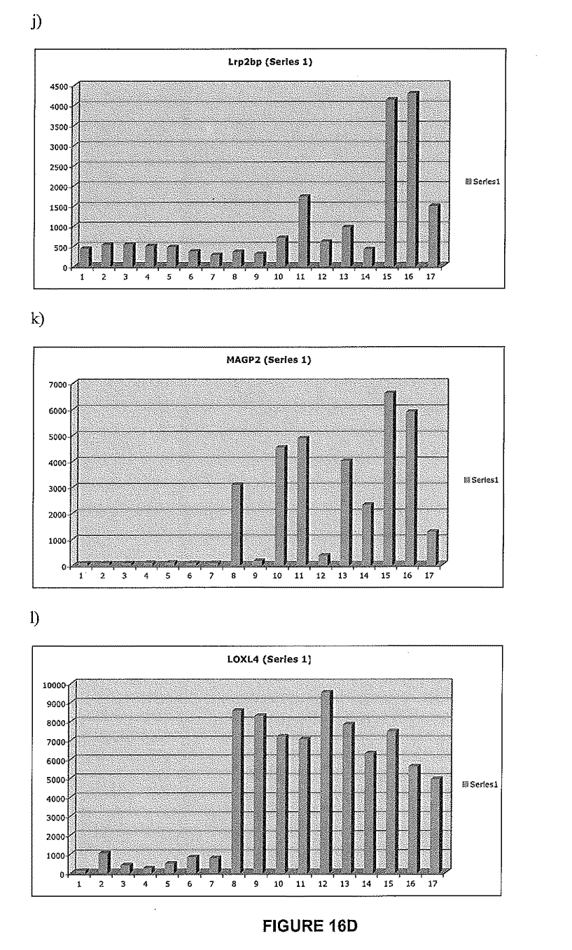

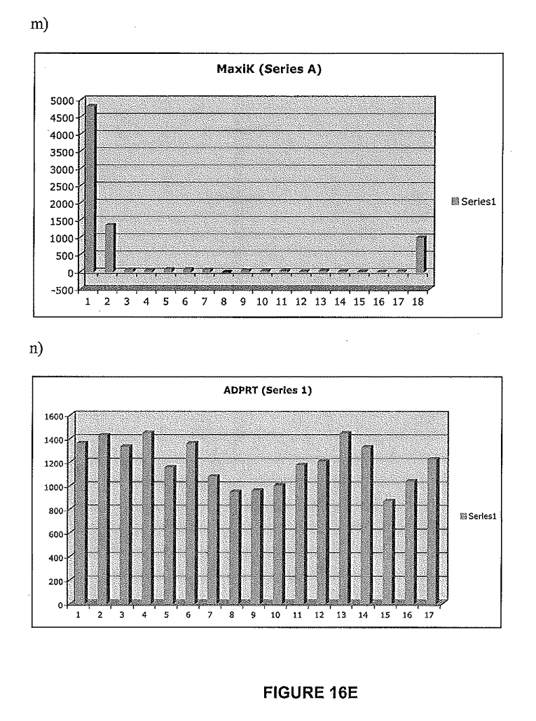

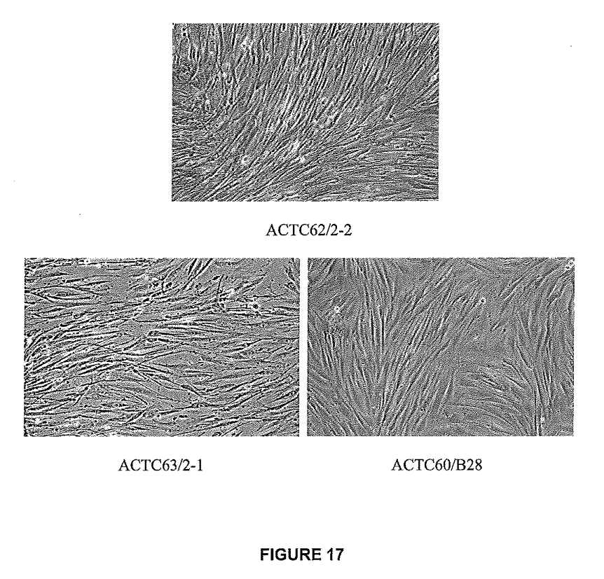

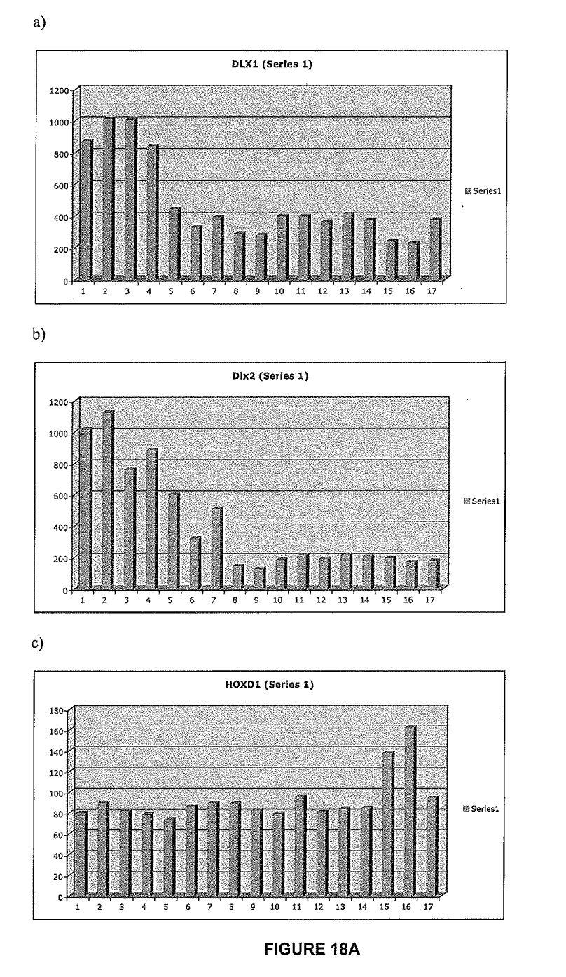

Clones 15 (2-2 or ACTC62), 16 (2-1 or ACTC63) and 17 (B28 or ACTC60) of Series 1 (see Example 17) displayed a pattern of gene expression consistent with smooth muscle progenitors and yet with numerous surprising genes being expressed with clones 15 and 16 of Series 1 displaying a pattern of large artery (aortic) vascular smooth muscle, and clone 17 of Series 1 showing a pattern of enteric smooth muscle in that the lines 15 and 16 expressed relatively high levels of expression of the smooth muscle actin gamma 2 (ACTAG2, Accession No. NM_{—}001615.2, smooth muscle actin (ACTA2, Accession No. NM_{—}001613.1), the endothelial receptor for angiopoietin-1 (TEK, Accession No. NM_{—}000459.1), tropomyosin-1 (TPM-1, Accession No. NM_{—}000366.4), calponin-1 (CNN1, Accession No. NM_{—}001299.3), the unidentified gene L0051063, the oxidized low-density (lectin-like) receptor-1 (OLM1), LRP2 binding protein (Lrp2 bp), MAGP2, LOXL4, and relatively low levels of expression of dysferlin, PLAP1, and MaxiK compared to the housekeeping gene ADPRT. The enteric smooth muscle clonogenic cell line 17 (also referred to as B-28 or ACTC60) showed markers for smooth muscle actin gamma 2, smooth muscle actin (ACTA2), the endothelial receptor for angiopoietin-1 (TEK), PLAP1, levels of tropomyosin-1 (TPM-1) comparable to fibroblast-like cells, calponin-1 (CNN1), LOXL4, MaxiK, and relatively low levels of expression of dysferlin, the unidentified gene L0051063, and OLR1, Lrp2 bp compared to the housekeeping gene ADPRT. See FIG. 16 which shows the relative expression of several of these markers in a data set normalized to other cell lines including those of Series 2. These, or a subset of any combination of these markers are useful in identifying or purifying these cells for use in research and therapy, such as for use in cell-based therapy. A phase contrast photograph of smooth muscle clonogenic cell lines is shown in FIG. 17.

The clonogenic cell line 17 of Series 1 (B-28 or ACTC60) (see Example 17) was deposited with the American Type Culture Collection (“ATCC”; P.O. Box 1549, Manassas, Va. 20108, USA) under the Budapest Treaty on Jun. 7, 2006, and have accession number ATCC PTA-7654. This cell line is an embryonic smooth muscle cell line with potential clinical application in heart disease, aneurysms and other age-related vascular disease, cancer, and intestinal disorders. See also Table X and XI for its CD antigen expression. Large vascular smooth muscle cells with an embryonic (prenatal) pattern of gene expression with high levels of elastogenesis as shown herein have clinical utility in the treatment of vascular disease such as strengthening the arterial wall by direct injection, or by IV injection, allowing the cells to home to sites of vascular lesions such as atheromas or aneurysms. These cells could be modified to carry therapeutic transgenes to the sites of malignancy. These cells could be injected into cardiac or skeletal muscle to strengthen the muscle. Also, particular splicing isoforms of the OLR1 gene known in the art (Biocca et al, Circ. Res. 97(2): 152-158 (2005)) could be introduced to these cells and the cells could then be protective against myocardial infarction, or to be use in the engineering of tissued engineered vascular tissue. Enteric smooth muscle cells are useful in strengthening the wall of the intestine, improving contractility, or the tissue engineering of intestinal tissue.

### Example 22

### The Use of Hox Gene Expression to Identify Clonogenic Cell Lines Derived from Pluripotent Stem Cells Such as hES Cells

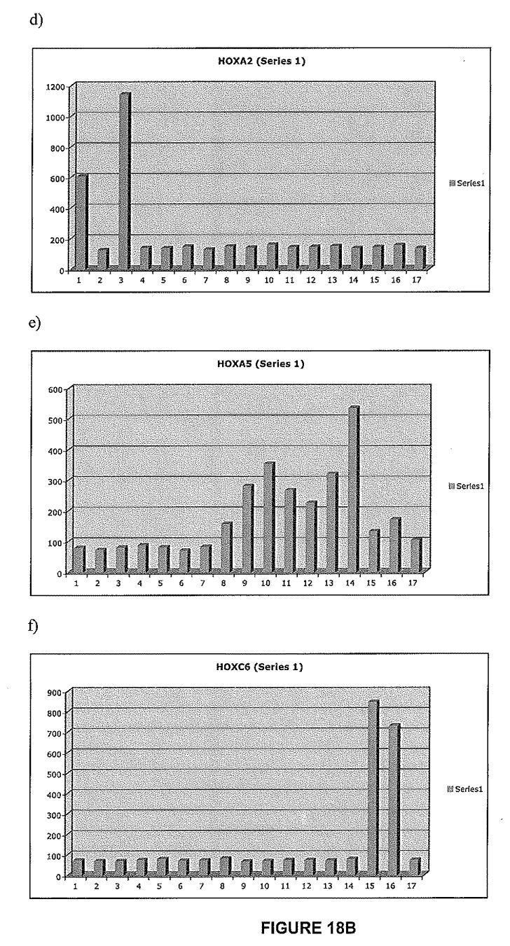

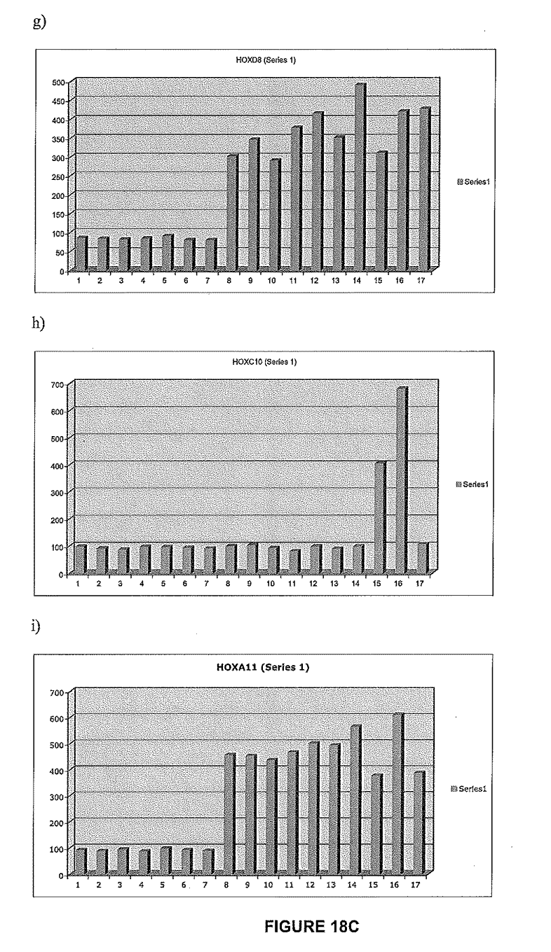

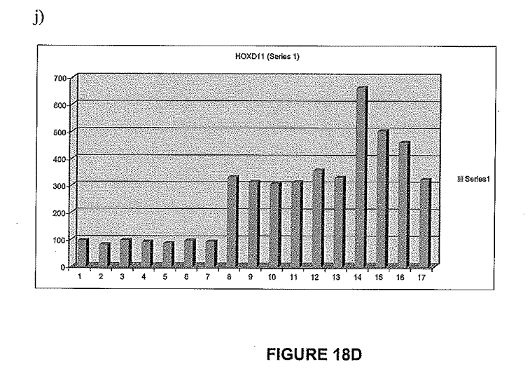

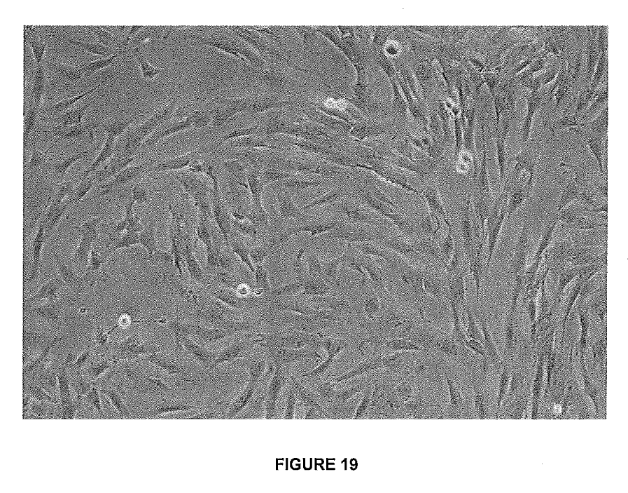

The expression of the Hox genes and other developmentally-regulated segmentation genes provide a useful marker of the origin of the clonogenic cell lines. This is generally not the case where the cells have a heterogeneous origin. By way of example, the cell clones described in example 17 above were compared for relative levels of genes such as the Hox genes and similar developmentally regulated segmentation genes. Those that displayed no expression are not shown. Shown in FIG. 18 are the expression of Dlx1, Dlx2. The expression of Dlx1 and Dlx2, but not Dlx3, Dlx5, Dlx6, or Dlx7, and the expression of HoxA2 and HoxB2 shows that cell clones 1, 3, and 7 of Series 1 (see Example 17) derive from the region of the third and fourth rhombomeres and would migrate to the region of about the dorsal first or more likely the second branchial arch. Clone 7 of Series 1 shows HoxB2 but not HoxA2 expression, confining the region of the cells to the junction of the third and fourth rhombomere. The smooth muscle cell clones 15 and 16 of Series 1 show HoxC6 and HoxC10 expression, consistent with these cells being of thoracic origin. The mesenchymal cell clones 8-14 of Series 1 including cell clone 8 with dermal progenitor characteristics, show HoxA10 and HoxA11 expression consistent with limb bud mesenchymal cells. Lastly, cell clone 17 of Series 1 with enteric smooth muscle characteristics has HoxA10 and HoxA11 expression but not HoxC6 or HoxC10 expression consistent with these cells deriving from somites in the lumbar region. The use of Hox and related developmentally-regulated segmentation genes to identify the nature of cell clones but also in matching the cells to the destination tissue insures that cells most suited for transplantation are obtained and used.

### Example 23

Induction of myocardial progenitors using inducer visceral endoderm cells. Visceral endoderm cells have an inductive effect on splanchnic mesoderm to differentiate into cells of the myocardial lineages. Pluripotent stem cells such as hES, hEC, hED, hEG or splanchnic mesoderm cells produced by the use of the methods of the present invention can be induced to differentiate into cells of cardiac lineages by juxtaposing said stem cells with visceral endoderm cells, including but not limited to cells expressing relatively high levels of AFP (Accession number NM_{—}001134.1) including 4-1, B10, 5, 4, B1, B27, 2, 4-4, B9, CM10-1, 4-2 (ACTC69), and 5-4 (ACTC68). In this example, hES cells are cultured as described herein, then three days following subculture, colonies are scraped from the dish and placed onto confluent cultures of visceral endoderm including those listed above and cultured in PromoCell Skeletal Muscle Medium (Table I, condition #1112) or its equivalent for 2-6 weeks. Myocardial cells can be identified by the use of markers well known in the art, including the presence of myocardial myosin heavy chain MYH7 (accession number NM_{—}000257.1).

### Example 24

hES cell colonies from one six well plate were grown to form embryoid bodies (EB) (see, e.g., U.S. application No. 60/538,964, filed Jan. 23, 2004, international patent publication no. WO05070011, published Aug. 4, 2005 and U.S. patent publication no. 20060018886, published Jan. 26, 2006, the disclosure of each of which is hereby incorporated by reference) and plated out to form epidermal keratinocytes that express a prenatal pattern of gene expression.

Specifically, colonies from the hES cell line H9 were differentiated by the removal of LIF-containing medium and the addition of DMEM medium containing 10% FBS. After 5 days of exposure to differentiation medium, the cells were trypsinized, and plated onto bacteriological plates and cultured for an additional 20 days to further induce differentiation as embryoid bodies. The cells were then trypsinized for 10 minutes with 0.25% trypsin/EDTA, neutralized with DMEM medium containing 10% FBS, counted with a Coulter counter, and the cells were plated at limiting dilutions from 5,000 plated cells, to 2,000 cells to 500 cells introduced into the 15 cm tissue culture plates with EpiLife medium (Cascade Biologics) Cat# M-EP/cf medium supplemented with calcium, LSGS (Cat#S-003-10) and recombinant collagen (Cat#R-011-K) per manufacturer's instructions. The cells were subsequently incubated in 5% ambient oxygen undisturbed for two weeks.

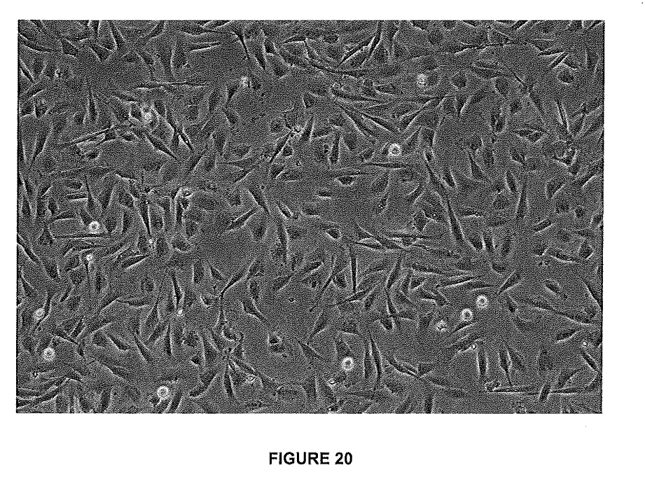

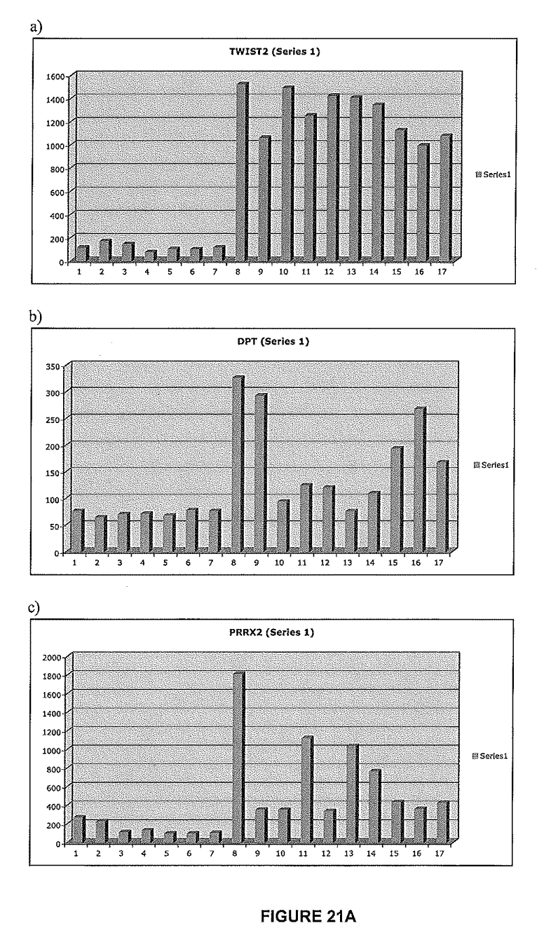

Clonal colonies were identified by phase contrast microscopy and those that are uniformly circular and well separated from surrounding colonies were marked for removal using cloning cylinders. A representative colony is shown in FIG. 20.

The trypsinized cells from within each cloning cylinder are then replated into collagen coated 24 well plates and incubated in the same medium until the cells reach confluency. Those that grow at a relatively rapid rate of approximately one doubling a day are then karyotyped to determine that they are normal human cells. A total genomic expression analysis using the Illumina system is then performed on the cells.

For improved wound repair, the keratinocytes with robust proliferative capacity are combined with dermal fibroblasts with a prenatal pattern of gene expression to produce skin equivalents capable of imparting a regenerative capacity to postnatal skin.

### Example 25

### Cranial Neural Crest Cells

Populations of neural crest cells of cranial, vagal, cardiac, or trunk origins can be derived according to the methods described in the present invention as these cells are formed in association with the differentiating central nervous system, neural tube and many differentiation conditions including in situ differentiation of hES, hEG, hiPS, hEC or hED cells, embryoid bodies formed from hES, hEG, hiPS, human EC or hED cells, or analogous differentiation systems that will form a complex mixture of neural tube-associated cells including the juxtaposition of neuroepithelium with inducing cells such as non-neural ectoderm (presumptive epidermis) in order to increase the number of neural crest progenitors or the administration of retinoic acid to shift the differentiation of neural crest types to a more caudal type. From heterogeneous mixtures of neural crest cells or neural crest progenitors, clonal or oligoclonal populations of the various neural crest cell types can be isolated according to the methods described in the present invention. Such cells may then be characterized through their pattern of gene expression or protein profiles to confirm their identity as neural crest cells. In the case of the human species and many species other than the laboratory mouse or chicken, the particular markers of various neural crest cells are not completely characterized.

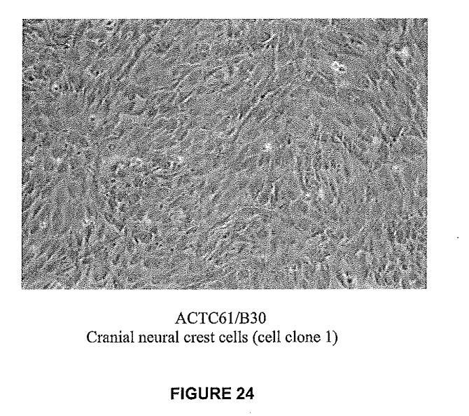

By way of nonlimiting example, example 17 of the present invention describes a method of obtaining clonal cranial neural crest cells from hES cells such as the hES cell line ACT3. Using the methods described in Example 17 above, single cell-derived cranial crest cells (also referred to as cell clone number 1 or ACTC61/B30 of Series 1) were generated. A phase contrast photograph of these cells at passage 7 is shown in FIG. 24.

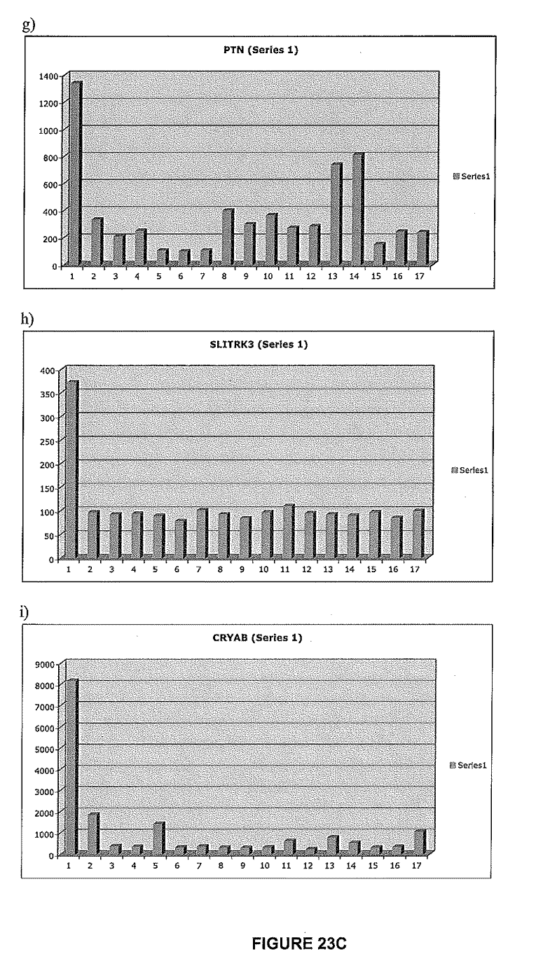

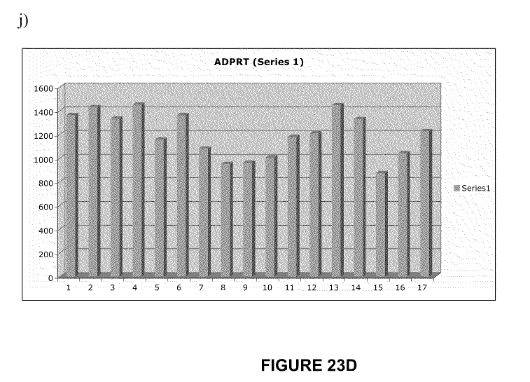

These cells displayed some but not all of the markers reported to correlate with mammalian cranial neural crest as well as novel and unexpected markers. The gene expression profile of cranial neural crest cell clone 1 is depicted in FIG. 23.

Cranial neural crest cells are well known to originate from the 1st-6th rhomomeres of the hindbrain. Depending on the rhombomere from which they originate, they differ in their expression of genes such as the HOX genes. Those originating from the third rhombomere express HOXA2 (Accession No. NM_{—}00673 5.3) and HOXB2, unlike the neural crest cells isolated from mice that express high levels of Sox10 (Sieber-Blum (2004) Dev. Dyn. 231:258-269). Surprisingly, cell clone number 1 (ACTC61/B30) was negative for SOX10 expression (data not shown) but did express SOX11 (Accession No. NM_{—}003108.3) (see FIG. 23). Similarly, cell clone number 1 of Series 1 (ACTC61/B30) did not express detectable levels of NCX (TLX2) expression, even though previous studies have reported that neural crest cells derived from mice and primates from ES cells are positive for this gene (Mizuseki et al (2003) PNAS 100(10):5823-5833) (data not shown). Other markers that distinguish the human cranial neural crest cell clone number 1 of Series 1 (ACTC61/B30) from other cell types include ID4 (Accession No. NM_{—}001546.2), FOXC1 (Accession No. NM_{—}001453.1), Cadherin-6 (Accession No. NM_{—}004932.2), PTN (Accession No. NM_{—}002825.5), SLITRK3 (Accession No. NM_{—}014926.2), and CRYAB (Accession No. NM_{—}0015885.1), as shown in FIG. 23. The relative expression levels of these markers normalized within the Series 1 data set are compared with the expression of the housekeeping ADPRT gene, as shown in FIG. 23.

The cranial neural crest cell clone 1 of Series 1 (ACTC61/B30) is also negative for HOXB1, HOXA3, HOXB3, HOXD3 and HOXB4 expression (data not shown). This further suggests that the cells originated from the third rhombomere and normally would have migrated into the second or third branchial arch largely at the level of the fourth rhombomere. Derivatives of the migrating cranial neural crest derived from the third and fifth rhombomeres stem from the region of the fourth rhombomere and migrate through the second branchial arch include bones such as the lesser horn of the hyoid bone, the stylohyoid ligament, the styloid process, and the stapes, muscles such as the buccinator, platysma, stapedius, stylohyoid, and the posterior belly of the digastric, and cranial nerve VII and are useful in regenerating numerous tissues as described herein.

Such cranial, vagal, cardiac or trunk neural crest cells can be used in a wide variety of applications in veterinary and human medicine for both research and therapeutic applications. By way of nonlimiting example, the cells may be used in either a nongenetically-modified or a genetically-modified form in cell-based assays for drug discovery, used to manufacture extracellular matrix materials or secreted factors such as cytokines, growth factors, and chemokines, or formulated and introduced into the bodies of humans or nonhuman animals in cell therapy to repair or regenerate tissues that these cells normally form in the embryo such as those listed above, or to deliver embryonic cytokines or growth factors such as to promote angiogenesis or neurite outgrowth as described herein.

The desired cell types can be differentiated from the neural crest stem cells by inducing differentiation and obtaining a population of cells enriched in a desired cell type, or by differentiating the neural crest cells into a heterogeneous mixture of downstream cell types and purifying out the desired cell type using techniques known in the art including genetic selection, or the use of affinity purification such as the use of antibodies or peptide ligands to antigens specific to the cell type of interest.

By way of nonlimiting examples, the methods to induce the differentiation of the neural crest cells may include the administration of 10 ng/mL of BMP2 for two weeks to generate chondrocytes, or 10 nM neuregulin-1 for two weeks to generate Schwann cells or peripheral neurons.

### Example 26

Another collection of clonal colonies from hES cells were generated. Methods of this invention are, and could be, used to generate these clonal colonies. These colonies represent the so-called Series 2 experiment. These cells are clonal colonies isolated from hES cells that have reduced differentiation potential than the starting parent hES cells.

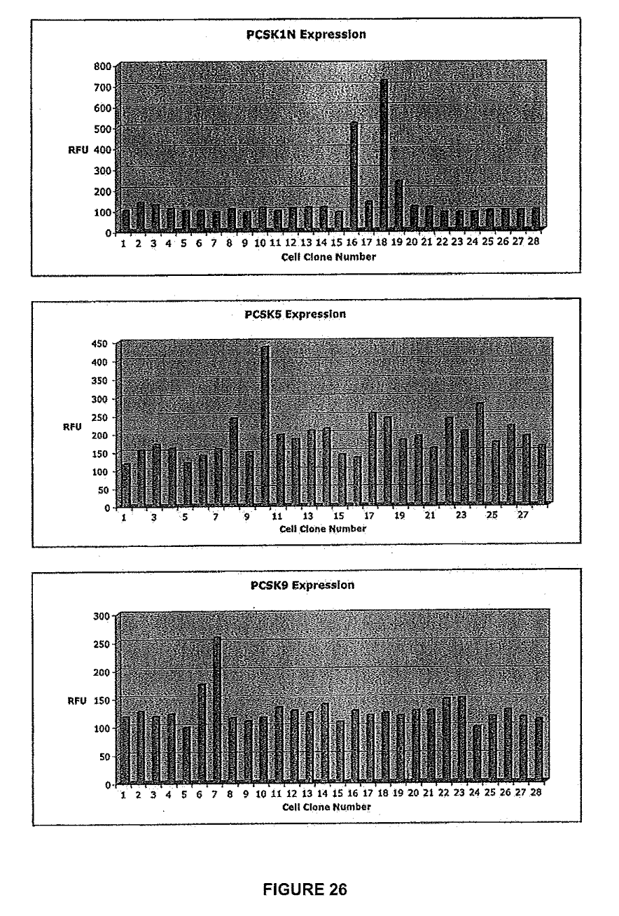

Of the colonies isolated from the Series 2 experiment, 28 colonies were studied. As shown in FIG. 26, normalized together with the data from the Series 1 experiment, the 28 clonal cell lines from Series 2 differentially expressed a number of genes that regulate prohormone convertases. In particular, the prohormone convertases (PCSK9, PCSK5) or the inhibitor of the prohormone convertase PC1 (PCSK1N), were shown to be overexpressed by some of the clonal cell lines from Series 2 (see FIG. 26). The expression of these markers could be plotted as relative expression to the ADPRT housekeeping gene.

Clones 16 and 18 of Series 2 expressed significant levels of PCSK1N (Accession No. NM_{—}013271.2), while clone 10 of Series 2 expressed significant levels of PCSK5 (Accession No. NM_{—}006200.2). Clones 6 and 7 of Series 2 also expressed significant levels of PCSK9 (Accession No. NM_{—}174936.2).

The expression of certain processing enzymes may play an important role during development by activating or inhibiting peptide hormones or growth factors that stimulate or inhibit differentiation. Therefore cell clones 16 and 18 may be used as a source of the PCSK1N protease to activate prohormones, and by analogy, other cell clones expressing other prohormone convertases may be used as a source of their respective convertases, or these convertases may be inhibited by peptides or other inhibitors to alter particular hormonal influences on cell growth or differentiation.

### Example 27

Some cell types do not proliferate well under any known cell culture conditions. To artificially stimulate the proliferation of such cells, the hES cell line H9 is transfected with a plasmid construct containing a temperature sensitive mutant of SV40 T antigen (Tag) regulated by a gamma-interferon promoter as described (Jat et al., Proc Natl Acad Sci USA 88:5096-5100 (1991)). The inducible Tag hES cells are then allowed to undergo a first step of differentiation with Tag in the uninduced state at the nonpermissive temperature of 37° C. and in medium lacking exogenous gamma-interferon in six differing conditions as follows.

Inducible Tag-expressing cells were plated in a standard 6 well tissue culture plate on a feeder layer of mouse embryonic fibroblasts and allowed to grow for 9 days to confluence. The hES cell growth medium was replaced by 6 extracellular matrix/growth media (see Table XVIII) and the hES cells were allowed to differentiate for 3 days.

The cells were trypsinized using 0.05% trypsin and transferred to Corning 6-well, ultra low attachment tissue culture plates containing the same differentiation medium. The embryoid bodies were allowed to differentiate for 7-10 days, collected, washed in phosphate buffered saline, dissociated into single cells with trypsin (0.25% trypsin) and the differentiated cells plated out in extra cellular matrix coated 15 cm plates (Table XVIII) in the same medium supplemented with gamma-interferon as described (Jat et al (1991) PNAS USA 88:5096-5100) under the permissive temperature of 32.5° C. The differentiated cells are allowed to proliferate for 14-20 days and the resulting colonies are cloned and plated in 24 well plates containing the same medium supplemented with gamma-interferon under the permissive temperature of 32.5° C. and extracellular matrix from which they were derived. The cloned colonies are expanded to obtain a stock of cells and the cell line stocks are cryopreserved. To determine the pattern of gene expression, the cells are shifted to the same medium reduced in serum concentration by 20-fold, free of gamma interferon, and at the nonpermissive temperature of 37° C. for five days.

TABLE XVIII
Extracellular Matrix & Growth Medium

| | | Extra |
|---|---|---|
| 15 cm | | Cellular |
| Plate | Selection & Growth Media | Matrix |
| 1 | Smooth Muscle Medium | Gelatin |
| 2 | Neurobasal Medium - B27 | Poly-lysine - |
| | | BioCoat |
| 3 | Epi-Life Medium - LSGS | Collagen IV |
| 4 | Endothelial Cell Growth Medium | Gelatin |
| 5 | Skeletal Muscle Cell Growth | Gelatin |
| | Medium | |
| 6 | DMEM + 10% FBS | Gelatin |

During the clonal expansion protocol, samples of the cell lines are taken for gene expression and immunophenotype analysis.

### Example 28

### Production of ED Endoderm and Pancreatic Beta Cells

Isolated blastomeres or similar ED cells such as isolated morula or ICM cells are isolated, as described in U.S. provisional Application No. 60/839,622, filed Aug. 23, 2006, its disclosure is hereby incorporated by reference. These cells are then added onto mitotically-inactivated feeder cells that express high levels of NODAL or cell lines that express members of the TGF-beta family that activate the same receptor as NODAL, such as CM02 cells that express relatively high levels of Activin-A, but low levels of Inhibins or follistatin. The cells are then incubated for a period of five days in DMEM medium with 0.5% human serum. After five days, the resulting cells which include definitive endodermal cells are purified by flow cytometry or other affinity-based cell separation techniques such as magnetic bead sorting using antibody specific to the CXCR4 receptor and then permeabilized and exposed to cellular extracts from isolated bovine pancreatic beta cells as described in U.S. patent publication 20050014258 (its disclosure being incorporated by reference). The resulting heterogeneous mixture of cells that has been induced toward beta cell differentiation is then cloned using techniques described herein. These cells are then directly differentiated into pancreatic beta cells or beta cell precursors using techniques known in the art for differentiating human embryonic stem cells into such cells or by culturing the hES cells on inducer cell mesodermal cell lines described herein.

### Example 29

### MicroRNA Profiles of Human Embryonic Stem Cells and Differentiated Progeny Cells

Isolation of total and miRNA from human embryonic stem cells and differentiated progeny cells. Total RNA or samples enriched for small RNA species were isolated from cell cultures that underwent serum starvation prior to harvesting RNA to approximate cellular growth arrest observed in many mature tissues. Cellular growth arrest was performed by changing to medium containing 0.5% serum for 5 days, with one medium change 2-3 days after the first addition of low serum medium. RNA were harvested according to the vendors instructions for Qiagen RNEasy kits to isolate total RNA or Ambion mirVana kits to isolate RNA enriched for small RNA species. The RNA concentrations were determined by spectrophotometry and RNA quality determined by denaturing agarose gel electrophoresis to visualize 28S and 18S RNA. Samples with clearly visible 28S and 18S bands without signs of degradation and at a ratio of approximately 2:1, 28S:18S, were used for subsequent miRNA analysis.

Assay for miRNA in samples isolated from human embryonic stem cells and differentiated progeny cells. The miRNAs were quantitated using a Human Panel TaqMan MicroRNA Assay from Applied Biosystems, Inc. This is a two step assay that uses stem-loop primers for reverse transcription (RT) followed by real-time TaqMan®. A total of 330 miRNA assays were performed to quantitate the levels of miRNA in the H9 human embryonic stein cell line, a differentiated fibroblast cell line, and nine cell lines differentiated from human embryonic stem cells. The assay includes two steps, reverse transcription (RT) and quantitative PCR (see FIG. 28). Real-time PCR was performed on an Applied Biosystems 7500 Real-Time PCR System. The copy number per cell was estimated based on the standard curve of synthetic mir-16 miRNA and assuming a total RNA mass of approximately 15 pg/cell.

The reverse transcription reaction was performed using 1×cDNA archiving buffer, 3.35 units MMLV reverse transcriptase, 5 mM each dNTP, 1.3 units AB RNase inhibitor, 2.5 nM 330-plex reverse primer (RP), 3 ng of cellular RNA in a final volume of 5 μl. The reverse transcription reaction was performed on a BioRad or MJ thermocycler with a cycling profile of 20° C. for 30 sec; 42° C. for 30 sec; 50° C. for 1 see, for 60 cycles followed by one cycle of 85° C. for 5 min.

This was followed by a pre-PCR amplification of reverse transcribed products. The 5 μl of reverse transcription reaction mixture was added to a mixture consisting of 1×UMM (no UNG) buffer, 50 nM 330-plex new forward primer (FP), 5 μM UR, 6.25 units AmpliTaqGold, 2 mM dNTP, 1 mM MgCl2 in a final volume of 25 μl. The pre-PCR reaction was performed on a BioRad or MJ thermocycler with a cycling profile of one cycle of 95° C. for 10 min, one cycle of 55° C. for 2 min; and 18 cycles of 95° C. for 1 sec, 65° C. for 1 min. The pre-PCR amplification mixture is subsequently diluted 1:4 by addition of 75 μl H₂O.

TaqMan quantitative PCR (qPCR) reactions were performed using 0.05 μl of diluted pre-PCR reaction mixture, 1×UMM(Fast), 500 nM FP, 200 nM TaqMan-probe, 500 nM UR in a final volume of 5 μl. The real time qPCR was performed on a Applied Biosystems 7500 FAST system using a cycling profile of one cycle of 95° C. for 10 min, followed by 40 cycles of 95° C. for 15 sec.; 60° C. for 1 min.

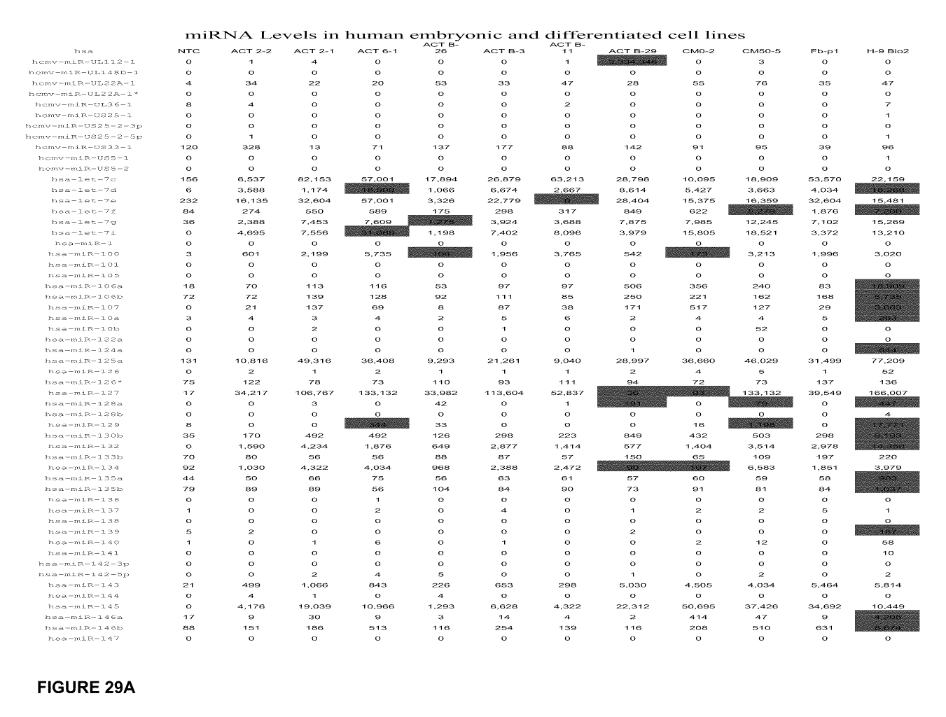

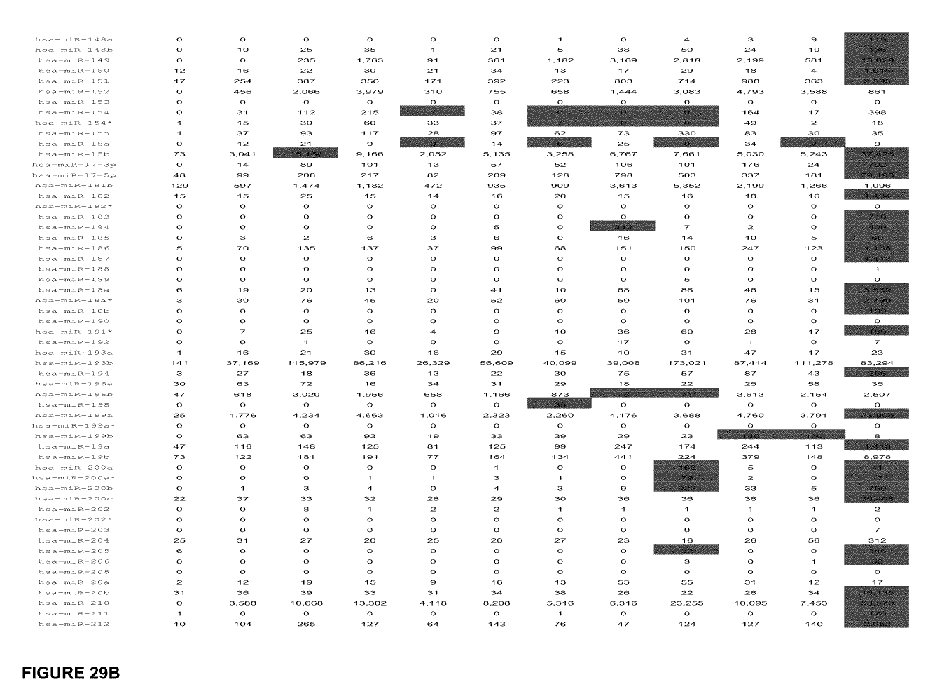

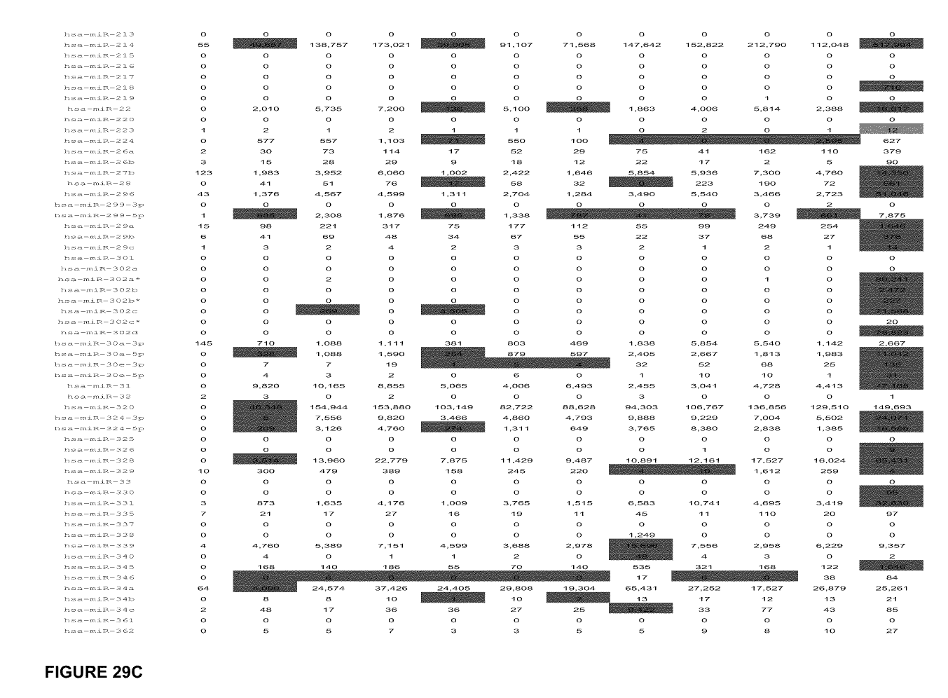

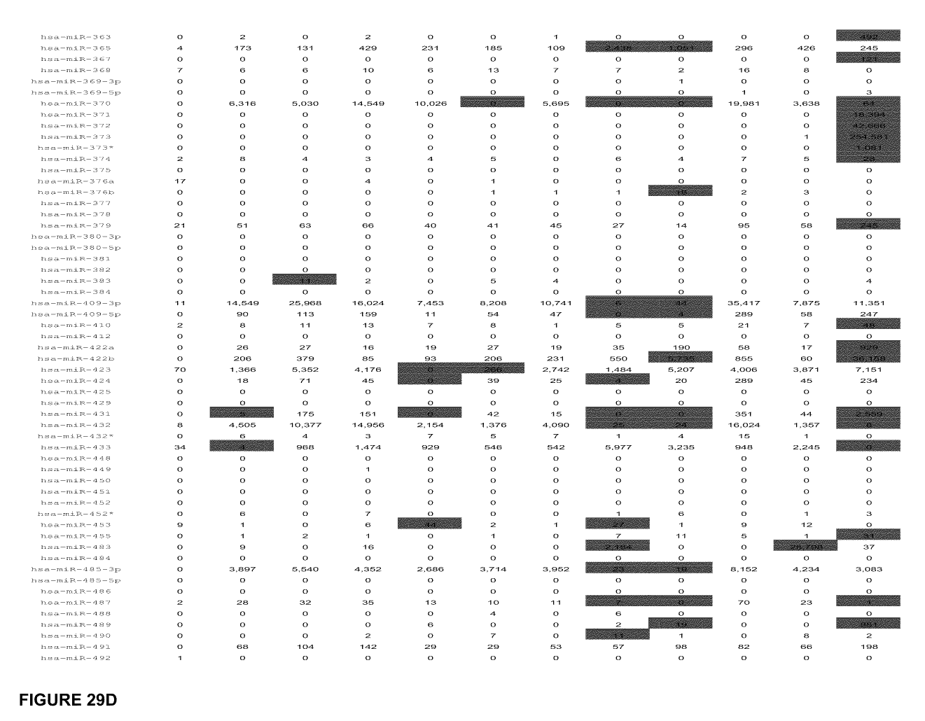

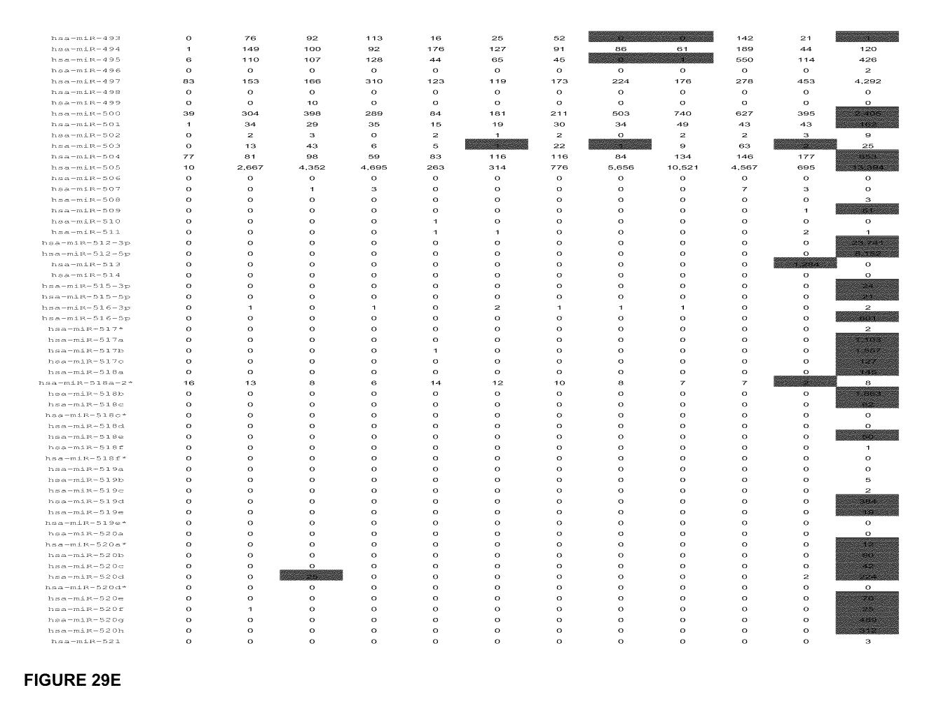

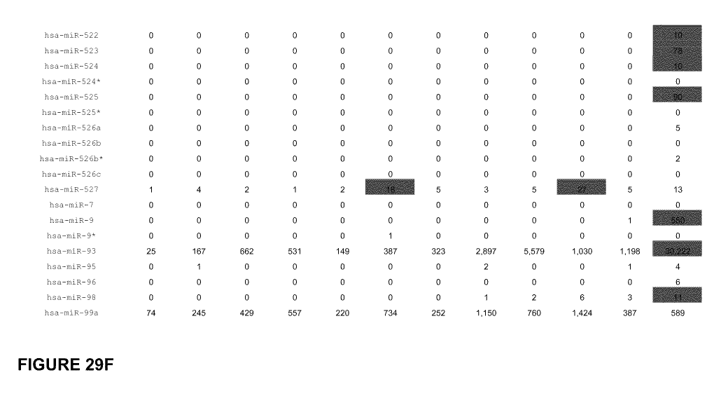

FIG. 29 summarizes the results of cellular miRNA levels in the H9 human embryonic stem cell line, the Fb-p1 fibroblast cell line and nine cell lines differentiated from parental human embryonic stem cells and shows unique miRNA profiles (red highlights) are apparent for all cell lines tested here.

### Example 30

### MicroRNA Expression Analysis from Single Cells Dissected from Tissue Samples

Cell lysate. Tissues from human embryos and adults are collected in DMEM (Gibco, Gaithersburg, Md.) with 0.5% BSA. Tissue fragments are cut out by a glass needle and incubated with 0.05% trypsin and 0.5 mM EDTA, followed by dissociation into single cells by a mouth pipette. Dissociated single cells are picked for miRNA expression analysis by several techniques including picking cells based on morphology, cell sorting or magnetic enrichment for cells expressing specific cell surface antigens, or by random picking. The entire process is performed as quickly as possible in order to minimize the effect of trypsin/EDTA treatment on gene expression. Subsequently, single cell suspensions are washed in 0.1% BSA in PBS twice. Washed single cells are individually introduced into RT reaction solution (without RT and dNTP) and treated at 95° C. for 5 min. Finally, RT, RNase Inhibitor and dNTP are added prior to the RT reaction.

Reverse transcription. One microlitre of total RNA or single cell lysate is used as template for a 5 μl reaction. RT reaction is carried out according to the manufacture's suggestions using the ABI high capacity cDNA archive kit (CN: 4322171). All primers and probes are designed based on miRNA sequences released by the Sanger Institute (http://microrna.sanger.ac.uk/sequences/). The primer and probe design is according to Chen et al. (Chen, C., Ridzon, D. A., Broomer, A. J., Zhou, Z., Lee, D. H., Nguyen, J. T., Barbisin, M., Xu, N. L., Mahuvakar, V. R., Andersen, M. R. et al. (2005) Real-time quantification of microRNAs by stem-loop RT-PCR. Nucleic Acids Res., 33, e179.). For example, for vmiR-16, the miRNA sequence is 5′-UAGCAGCACGUAAAUAUUGGCG-3′. The reverse primer is 5′-CTCAACTGGTGTCGTGGAGTCGGCAATTCAGTTGAGCGCCAATA-3′. The forward primer is 5′-ACACTCCAGCTGGGTAGCAGCACGTAAATA-3′. The TaqMan Probe is (6-FAM)TTCAGTTGAGCGCCAATA (MGB; MGB is a minor grove binder with non fluorescent quencher). For miR-293, the miRNA sequence is 5′-AGUGCCGCAGAGUUUGUAGUGU-3′. The reverse primer is 5′-CTCAACTGGTGTCGTGGAGTCGGCAATTCAGTTGAGACACTACA-3′. The forward primer is 5′-ACACTCCAGCTGGG AGTGCCGCAGAGTTTG-3′. The TaqMan Probe is (6-FAM)TTCAGTTGAGACACTACA (MGB). Briefly, mixtures of 5 nM of each of the 330 miRNA specific reverse primer together with 1.3 U RNase Inhibitor, 16.75 U MMLV RT and 25 M dNTP are used for each RT reaction. The potential non-specific interactions between the looped primers are reduced by using 10-fold less looped primer concentration compared with amounts used in 1-plex looped RT-PCR assay (i.e. 5 nM of each primer instead of 50 nM). All 330 miRNAs are converted into corresponding cDNAs in one RT reaction. A pulsed RT reaction condition is used to increase RT efficiency and further reduce non-specific interactions between primers for different miRNAs. The pulsed RT reaction condition gives 0.5-1 lower Ct value which means better detection sensitivity compared with non-pulsed condition used in 1-plex looped RT-PCR assay. However, there is no amplification of the miRNA cDNAs at this step. The reaction condition is as follows: 16° C. for 30 min, followed by 60 cycles at 20° C. for 30 s, 42° C. for 30 s and 50° C. for 1 s. A final incubation at 85° C. for 5 min is used to inactivate MMLV RT.

Pre-PCR. RT product (5 μl) is used as template for a 25 ul PCR. Briefly, 50 nM of each of the 330 miRNA's Forward Primers, 1× TaqMan Universal Master Mix (ABI), 4 mM dNTP, 2 mM MgCl2, 5 uM Universal Reverse Primer, 6.25 U AmpliGold Taq (ABI) are used for each Pre-PCR. The condition for the PCR is 95° C. for 10 min, 55° C. for 2 min, followed by 18 cycles of 95° C. for 1 s and 65° C. for 1 min. Pre-PCR is an essential step for the 330-plex assay, since without this step there is significant loss of detection sensitivity, and most miRNAs will not be detectable except for those that are expressed at high levels in single cell inputs.

Real-time PCR. Two microlitres of 1:400 diluted Pre-PCR product is used for a 20 ul reaction. All reactions are duplicated. Because the method is very robust, duplicate samples are sufficient and accurate enough to obtain values for miRNA expression levels. TaqMan universal PCR master mix of ABI is used according to manufacture's suggestion. Briefly, 1× TaqMan Universal Master Mix (ABI), 1 uM Forward Primer, 1 uM Universal Reverse Primer and 0.2 uM TaqMan Probe is used for each real-time PCR. The conditions used are as follows: 95° C. for 10 min, followed by 40 cycles at 95° C. for 15 s, and 60° C. for 1 min. All the reactions are run on ABI Prism 7000 Sequence Detection System.

FIG. 30 depicts a schematic representation of real-time PCR-based 330-plex microRNA expression profiling method as described above.

### Example 31

### Gene Expression Analysis from Single Cells Dissected from Tissue Samples

cDNA synthesis from single cells or single-cell level total RNA. Total RNA is purified from cells using the RNeasy Mini kit (Qiagen, Hilden, Germany). For preparation of diluted RNA, we serially dilute the total RNA of approximately 1000 ng/ml to concentrations of 2.5 ng/ul, 250 pg/μl and 25 pg/μl. Then, 0.4 μl (10 pg) of the final dilution (25 pg/μl) is directly added to single-cell lysis buffer (see below).

Tissues from human embryos and adults are collected in DMEM (Gibco, Gaithersburg, Md.) with 0.5% BSA. Tissue fragments are cut out by a glass needle and incubated with 0.05% trypsin and 0.5 mM EDTA, followed by dissociation into single cells by a mouth pipette. Dissociated single cells are picked for single-cell cDNA synthesis by several techniques including picking cells based on morphology, cell sorting or magnetic enrichment for cells expressing specific cell surface antigens, or by random picking. The entire process is performed as quickly as possible in order to minimize the effect of trypsin/EDTA treatment on gene expression.

Isolated single cells, or a single-cell equivalent amount of RNA, are seeded into 0.5 ml thin-walled PCR tubes containing 4.5 ml of cell lysis buffer [1×PCR buffer II (Applied Biosystems, Foster City, Calif.), 1.5 mM MgCl₂ (Applied Biosystems), 0.5% NP40, 5 mM DTT, 0.3 U/μl Prime RNase Inhibitor (Eppendorf, Hamburg, Germany), 0.3 U/μl RNAguard RNase Inhibitor (Amersham Biosciences, Piscataway, N.J.), 0.2 ng/μl primer V1(dT)24 and 0.05 mM each of dATP, dCTP, dGTP and dTTP], containing an appropriate amounts of spike RNAs (see below). The sequence of the V1 (dT)24 primer is 5′-ATATGGATCCGGCGCGCCGTCGACTTTTTTTTTTTTTTTTTTTTTTTT-3′. All the primers described in this paper are purchased from Operon Biotechnology (Huntsville, Ala.). After 15 s centrifugation, cell lysis is performed at 70° C. for 90 s, and the reaction tubes are immediately put on ice for 1 min. A 0.3 μl volume of RT mixture [133.3 U/μl SuperScript III (Invitrogen), 3.33 U/μl RNAguard RNase Inhibitor (Invitrogen, Carlsbad, Calif.), and 1.1-1.3 μg/μl T4 gene 32 protein (Roche, Basel, Switzerland)] are added to each reaction tube. The reaction mixture is incubated at 50° C. for 5 min and heat-inactivated at 70° C. for 10 min. The tubes are immediately put on ice for 1 min, and after 15 s centrifugation, 1.0 ul of Exonuclease I mixture [1× Exonuclease I buffer (Takara, Shiga, Japan) and 0.5 U/μl Exonuclease I (Takara)] is added to each tube. The reaction mixture is incubated at 37° C. for 30 min and heat-inactivated at 80° C. for 25 min. The reaction tubes are then put on ice for 1 min. Poly-A tails are synthesized on the reverse transcribed molecules by adding 6 μl of terminal deoxynucleotidyl transferase (TdT) mixture [1×PCR buffer II, 1.5 mM MgCl₂, 3 mM dATP, 0.1 U/μl RNaseH (Invitrogen) and 0.75 U/ul TdT (Invitrogen)] to each tube, and the mixture incubated at 37° C. for 15 min followed by heat-inactivation at 70° C. for 10 min. The synthesized poly(dA)-tailed RT product in each tube (12 μl) is divided into four 0.2 ml thin-walled PCR tubes (3 μl each). Then, 19 μl of PCR mixture I [1× ExTaq buffer, 0.25 mM each of dATP, dCTP, dGTP and dTTP, 0.02 μg/μl primer V3 (dT)24, and 0.05 U/μl ExTaq Hot Start Version (Takara)] is added to each tube for the first round of PCR: 95° C. for 3 min, 50° C. for 2 min and 72° C. for 3 min. The sequence of V3 (dT)24 is 5′-ATATCTCGAGGGCGCGCCGGATCCTTTTTTTTTTTTTTTTTTTTTTTT-3′. The tubes are immediately put on ice for 1 min, and 19 μl of PCR mixture II is added, with a composition similar to that of PCR buffer I but with primer V1 (dT)24 replacing primer V3 (dT)24. A drop of mineral oil (Sigma-Aldrich, St Louis, Mo.) is added to each tube. A 20-cycle PCR amplification is performed according to the following schedule: 95° C. for 30 s, 67° C. for 1 min and 72° C. for 3 min with a 6 s extension per cycle. The amplified cDNA is purified with a QIAquick PCR kit (Qiagen) and dissolved in 50 μl of buffer EB (10 mM Tris-HCl, pH 8.5). The cDNA products are subjected to another amplification step to allocate the T7 promoter sequence at the 5′-terminus. A 49.4 μl volume of PCR mixture III [1× ExTaq buffer, 0.25 mM each of dATP, dCTP, dGTP and dTTP, 0.02 ug/ul primer T7-V1 (5′-GGCCAGTGAATTGTAATACGACTCACTATAGGGAGGCGGATATGGATCCGGCGCGCCGTCGAC-3), 0.02 μg/μl primer V3 (dT)24 and 0.05 U/μl ExTaq Hot Start Version] is added to each of eight 0.2-ml thin-walled PCR tubes containing 0.63 μl of the 20 cycle amplified cDNA. A nine-cycle amplification is then performed according to the following schedule: 95° C. for 5 min 30 s, 64° C. for 1 min and 72° C. for 5 min 18 s for the first cycle; and 95° C. for 30 s, 67° C. for 1 min and 72° C. for 5 min 18 s with an extension of 6 s per cycle for another eight cycles. The products are mixed together after the reaction, purified with a QIAquick PCR purification kit, and dissolved in 30 μl of buffer EB. The PCR product is purified with 2% agarose gel electrophoresis to remove by-product DNA shorter than 300 bp. The cDNA is extracted from a gel fragment with a QIAquick Gel Extraction kit (Qiagen) and dissolved in 35 μl of buffer EB. A 47.8 μl volume of PCR mixture III is added to each of four 0.2 ml thin-walled PCR tubes containing 2.2 μl of the purified cDNA, and an additional one-cycle PCR (95° C. for 5 min 30 s, 67° C. for 1 min and 72° C. for 16 min) is performed. The products are mixed together after the reaction, purified with the QIAquick PCR purification kit, and dissolved in 30 μl of buffer EB. To prepare the spike RNAs, Escherichia coli cells containing plasmids encoding poly(A)-tailed Bacillus subtilis lys, phe, thr, and dap genes are purchased from the American Type Culture Collection (ATCC, Manassas, Va.; the ATCC numbers are 87482, 87483, 87484 and 87486, respectively). The sense-strand RNAs are synthesized with the MEGAscript T3 kit (Ambion, Austin, Tex.) and purified with the RNeasy Mini kit. An appropriate amount of spike RNA mixture is added to the cell lysis buffer and to 5 ug (5×10⁵ cells) of total RNA for the microarray experiments, so that the reaction mixture contained poly(A)-tailed Lys, Dap, Phe and Thr RNAs at 1000, 100, 20 and 5 copies per cell, respectively.

Microarray hybridization and data processing. Eight independently amplified cDNA samples and cellular total RNA (5 μg in each of eight individual tubes) are subjected to the One-Cycle Target Labeling procedure for biotin labeling by in vitro transcription (IVT) (Affymetrix, Santa Clara, Calif.) or using the Illumina Total Prep RNA Labelling kit. For analysis on Affymetrix gene chips, the cRNA is subsequently fragmented and hybridized to the Human Genome U133 Plus 2.0 Array (Affymetrix) according to the manufacturer's instructions. The microarray image data are processed with the GeneChip Scanner 3000 (Affymetrix) to generate CEL data. The CEL data are then subjected to analysis with dChip software, which has the advantage of normalizing and processing multiple datasets simultaneously. Data obtained from the eight nonamplified controls from cells, from the eight independently amplified samples from the diluted cellular RNA, and from the amplified cDNA samples from 20 single cells are normalized separately within the respective groups, according to the program's default setting. The model based expression indices (MBEI) are calculated using the PM/MM difference mode with log-2 transformation of signal intensity and truncation of low values to zero. The absolute calls (Present, Marginal and Absent) are calculated by the Affymetrix Microarray Software 5.0 (MAS 5.0) algorithm using the dChip default setting. The expression levels of only the Present probes are considered for all quantitative analyses described below. The GEO accession number for the microarray data is GSE4309. For analysis on Illumina Human Sentrix 6 Bead Chips, labeled cRNA are hybridized according to the manufacturer's instructions.

Calculation of coverage and accuracy. A true positive is defined as probes called Present in at least six of the eight nonamplified controls, and the true expression levels are defined as the log-averaged expression levels of the Present probes. The definition of coverage is (the number of truly positive probes detected in amplified samples)/(the number of truly positive probes). The definition of accuracy is (the number of truly positive probes detected in amplified samples)/(the number of probes detected in amplified samples). The expression levels of the amplified and nonamplified samples are divided by the class interval of 20.5 (20, 20.5, 21, 21.5 . . . ), where accuracy and coverage are calculated. These expression level bins are also used to analyze the frequency distribution of the detected probes.

Analysis of gene expression profiles of cells. The unsupervised clustering and class neighbor analyses of the microarray data from cells are performed using GenePattern software (http://www.broad.mit.edu/caneer/software/genepattern/), which performs the signal-to-noise ratio analysis/T-test in conjunction with the permutation test to preclude the contribution of any sample variability, including those from methodology and/or biopsy, at high confidence. The analyses are conducted on the 14 128 probes for which at least 6 out of 20 single cells provided Present calls and at least 1 out of 20 samples provided expression levels >20 copies per cell. The expression levels calculated for probes with Absent/Marginal calls were truncated to zero. To calculate relative gene expression levels, the Ct values obtained with Q-PCR analyses are corrected using the efficiencies of the individual primer pairs quantified either with whole human genome (BD. Biosciences) or plasmids that contain gene fragments. The relative expression levels are further transformed into copy numbers with a calibration line calculated using the spike RNAs included in the reaction mixture (log 10[expression level]=1.05×log 10[copy number]+4.65). The Chi-square test for independence is performed to evaluate the association of gene expressions with Gata4, which represents the difference between cluster 1 and cluster 2 determined by the unsupervised clustering and which is restricted to PE at later stages. The expression levels of individual genes measured with Q-PCR are classified into three categories: high (>100 copies per cell), middle (10-100 copies per cell), and low (<10 copies per cell). The Chi-square and P-values for independence from Gata4 expression are calculated based on this classification. Chisquared is defined as follows: χ²=ΣΣ(n fij−fi fj)²/n fi fj, where i and j represent expression level categories (high, middle or low) of the reference (Gata4) and the target gene, respectively; fi, fj, and fij represent the observed frequency of categories i, j and ij, respectively; and n represents the sample number (n=24). The degrees of freedom are defined as (r−1)×(c−1), where r and c represent available numbers of expression level categories of Gata4 and of the target gene, respectively.

### Example 32

### Pleiotrophin and Midkine-Expressing Cell Lines

Cell lines expressing the factors pleiotrophin (PIN; Accession number NM_{—}002825.5) and/or midkine (MDK; Accession number NM_{—}002391.2) have unique uses in inducing angiogenesis and/or in imparting neuroactive effects such as inhibiting apoptosis following injury to neurons including retinal neurons. The cell line Z11 (ACTC194) derived as described herein in Example 36 expresses high levels of both PTN and MDK Z11 also expresses high levels of the angiogenic factor angiopoietin 2 (ANGPT2; Accession number NM_{—}001147.1). Therefore, Z11 (ACTC194) is useful in delivering these factors via cell therapy in vivo as described herein to impart angiogenic and neurotrophic activity. Other cell lines expressing relatively high levels of PTN include: B30 (ACTC61), ELS5-6 (ACTC118), MEL2, C4ELSR_{—}1, E75 (ACTC102), E72 (ACTC100), B7 (ACTC53), 6-1 (ACTC64), B2 (ACTC51), B25 (ACTC57), B26 (ACTC50), B4 (ACTC66), E111, 6, B17 (ACTC54), SM28 (ACTC150), SK17 (ACTC162), Z8 (ACTC213), Z7 (ACTC200), SM2 (ACTC142), SM49 (ACTC151), EN11 (ACTC215), W10 (ACTC196), EN2 (ACTC139), SM22 (ACTC156), EN55 (ACTC185), EN4 (ACTC144), EN42 (ACTC175), W11 (ACTC197), SK18 (ACTC158), EN28, and EN38 (ACTC202) and the conditions for isolating and propagating are described in the instant application. Other cell lines expressing relatively high levels of MDK include: J13 (ACTC172), MEL2, 5, E75 (ACTC102), E72 (ACTC100), 2-2 (ACTC62), 6-1 (ACTC64), B2 (ACTC51), 2-1 (ACTC63), B25 (ACTC57), B26 (ACTC50), B11 (ACTC58), B3 (ACTC55), B30 (ACTC61), B6 (ACTC56), B17 (ACTC54), B29 (ACTC52), SM8, SK17 (ACTC162), EN7, EN13 (ACTC174), SK5, SM25 (ACTC166), Z8 (ACTC213), SM17 (ACTC182), SM33 (ACTC183), SM4 (ACTC143), Z7 (ACTC200), SM2 (ACTC142), SK50 (ACTC159), SM49 (ACTC151), EN11 (ACTC215), W10 (ACTC196), EN2 (ACTC139), SM22 (ACTC156), EN55 (ACTC185), EN26 (ACTC140), EN27 (ACTC199), EN4 (ACTC144), EN42 (ACTC175), W11 (ACTC197), SK18 (ACTC158), SK46 (ACTC137), EN28, EN47 (ACTC176), and EN31 (ACTC141) and the conditions for isolating and propagating are described in the instant application.

These cells described in this example expressing PTN or MDK may be injected directly into tissues to impart an angiogenic or neuroprotective effect, or alternatively, they may be formulated on or in a matrix including but not limited to a practical device configuration for releasing secreted factors such as cell encapsulation. The cells can be encapsulated (or microencapsulated) collectively or as clusters or individually in porous implantable polynmeric capsules. These can be made of a variety of substances, including but not limited to polysaccharide hydrogels, chitosans, calcium or barium alginates, layered matrices of alginate and polylysine, poly(ethylene glycol) (PEG) polymers, polyacrylates (e.g., hydroxyethyl methacrylate methyl methacrylate), silicon, or polymembranes (e.g., acrylonitrile-co-vinyl chloride) in capillary-like, tube-like or bag-like configurations. Among the requirements for therapeutic utility are chemical definability, the ability to validate structure, stability, resistance to protein absorption, lack of toxicity, permeability to oxygen and nutrients as well as to the released therapeutic compounds, and resistance to antibodies or cellular attack. In addition, the cells may be mitotically inactivated such as with a typical irradiation protocol for this purpose such as exposing the cells to 20 to 50 Gy (2000 to 5000 rads; sometimes up to 100 Gy) from a Cs-137 or C0-60 source as is well-known in the art. Alternatively, such cells may be mitotically inactivated by other means including, but not limited to DNA-damaging molecules such as mitomycin C. A typical protocol using mitomycin C to inactivate the cells would be:

Mitomycin C Treatment of Cells

1. Grow cells to confluence in 15 cm plates or T-150 flasks. 2. Inject 2 ml of sterile water (or PBS) into Mitomycin C (Sigma, Cat# M4287-2MG) vial and dissolve completely. Concentration of Mitomycin C is 1 mg/ml. Once prepared, Mitomycin C is good for about 2 weeks when stored at 4 C. 3. Prepare about 10 ml of warm medium for each plate or flask. Add 100 ul of Mitomycin C to each 10 ml of medium. Concentration of Mitomycin C is 10 ug/ml. 4. Aspirate medium from the plates or flasks and replace with the Mitomycin C medium (10 ml per plate or flask). Place in CO₂ incubator at 37 C for 3 hours. 5. Aspirate Mitomycin C medium into disposal trap that containing bleach. Wash Mitomycin C treated cells 2-4 times with warm PBS. Aspirate PBS into bleach containing trap. 6. Trypsinize cells, neutralize the Trypsin with DMEM+10% FBS and count the number of cells with a Coulter Counter or hemacytometer. 7. Determine the number of cells needed to cover the vessel of interest. For example, for mouse embryonic fibroblasts (MEF) feeder cells, at least 500K cells are needed for one well of a 6 well plate. Increase this cell number by approximately 10-30% to account for cell death during the freezing process. 7. Freeze the cells in aliquots convenient for later use. For example, MEF feeder cells can be frozen in aliquots for single wells (650K), 3 wells (1.75 million) or 6 wells (3.3 million). Freezing medium is the same medium used to grow the cells containing 10% dimethylsulfoxide (DMSO) and freezing solution should be cooled to 2-4 C prior to use. Do not use DMSO freezing medium warmed to 37 C. Medium should contain at least 10% serum for best results. 8. Before discarding any unused Mitomycin C or vessels used in the inactivation procedure, treat with bleach.

### Example 33

### Derivation of Initial Heterogeneity in 5% FBS DMEM

In this series of novel cell line derivation known as series EB3, initial differentiation and generation of heterogeneity was performed in 5% FBS containing DMEM (Table I, conditions 455 and 1103). H9 human embryonic stem (hES) cells were routinely cultured in hES medium (KO-DMEM, 1× nonessential amino acids, 1× Glutamax-1, 55 μM beta-mercaptoethanol, 10% Serum Replacement, 10% Plasmanate, 10 ng/ml LIF, 4 ng/ml bFGF, and penicillin/streptomycin) and passaged by manual dissection. Except where indicated, all tissue culture plastic wares were coated with 0.1% gelatin. Before processing cells to make embryoid bodies, H9 hES cells were cultured for 2 days in DMEM 5% fetal bovine serum (FBS) supplemented with penicillin/streptomycin. To process cells to make embryoid bodies, 119 hES cells were harvested by manual dissection of individual colonies, the cell-clump suspension was replated into non-coated 10 cm plastic bacterial Petri dishes in DMEM 5% FBS and cultured for 7 days at 37 deg C. (10% CO2, 5% O2). Unattached bulk embryoid bodies were harvested by aspirating growth medium and attached cells were harvested by trypsinization and pooled with unattached bulk embryoid bodies. Cells were concentrated by centrifugation and plated for the second step of clonal isolation into 6 well tissue culture dishes in either DMEM 20% FBS (Table I, conditions 457 and 1103, PromoCell Skeletal Muscle Cell Growth medium (Table I, condition 1112), PromoCell Smooth Muscle Cell Growth medium (Table I, condition 1113), PromoCell Endothelial Cell Growth medium (Table I condition 1110), Stem Cell Technology Mesenchymal medium (Table I, condition 1114), or EpiLife LSGS medium (Table I, condition 1109), each supplemented with penicillin/streptomycin (Table I conditions 1127 and 1128). Cells were serially grown in 6 well, and 10 cm tissue culture dishes and finally replated at a density of approximately 1000 to 2000 cells/15 cm tissue culture dish in their respective media with penicillin/streptomycin. In the case of cells grown in EpiLife LSGS medium, the cells were plated at relatively high densities of 2000, 5000 and 10,000 cells/15 cm tissue culture dish. After approximately two weeks of growth in either DMEM 20% FBS, PromoCell Skeletal Muscle Cell Growth medium, PromoCell Smooth Muscle Cell Growth medium, or PromoCell Endothelial Cell Growth medium, colonies were picked. In the case of cells grown in EpiLife LSGS medium, cells were incubated for approximately three months before colonies were picked. Colonies were serially grown in 24 well, 12 well, 6 well tissue culture plates, T25, T75, T150 flasks, and 2 liter Roller Bottles (850 cm² surface area) before freezing and storage in liquid nitrogen. Cell morphologies and cell growth were monitored by phase contract microscopy and recorded by photomicroscopy. Cells were cultured in 6 well tissue culture plates or 6 cm tissue culture Petri dishes to harvest RNA for gene expression analysis using the Illumina human sentra-6 platform.

EB(3) Experiment (252 total colonies picked)

| Line | ACTC | | Line | ACTC | | Line | ACTC | | Line | ACTC | | Line | ACTC | |
|---|---|---|---|---|---|---|---|---|---|---|---|---|---|---|
| Name | No. | Medium | Name | No. | Medium | Name | No. | Medium | Name | No. | Medium | Name | No. | Medium |
| SK1 | 203 | Skeletal | EN1 | 173 | PromoCell | SM1 | | Smooth | DM1 | | DMEM + | ME1 | | Mesenchymal |
| SK2 | | Muscle | EN2 | 139 | Endo- | SM2 | 142 | Muscle | DM2 | | 20% Fetal | ME2 | | Medium |
| SK3 | 168 | | EN3 | | thelial | SM3 | | | DM3 | | Bovine | ME3 | | |
| SK4 | | | EN4 | 144 | Medium | SM4 | 143 | | DM4 | | Serum | ME4 | | |
| SK5 | 157 | | EN5 | 145 | | SM5 | | | DM5 | | | ME5 | | |
| SK6 | | | EN6 | | | SM6 | | | DM6 | | | ME6 | | |
| SK7 | | | EN7 | 184 | | SM7 | | | DM7 | | | ME7 | | |
| SK8 | 190 | | EN8 | 249 | | SM8 | 225 | | DM8 | | | ME8 | | |
| SK9 | | | EN9 | 234 | | SM9 | | | DM9 | | | ME9 | | |
| SK10 | 219 | | EN10 | | | SM10 | | | DM10 | | | ME10 | | |
| SK11 | 250 | | EN11 | 215 | | SM11 | | | DM11 | | | ME11 | | |
| SK12 | | | EN12 | | | SM12 | | | DM12 | | | ME12 | | |
| SK13 | | | EN13 | 174 | | SM13 | | | DM13 | | | ME13 | | |
| SK14 | 218 | | EN14 | | | SM14 | | | DM14 | | | ME14 | | |
| SK15 | | | EN15 | | | SM15 | | | DM15 | | | ME15 | | |
| SK16 | | | EN16 | 221 | | SM16 | | | DM16 | | | ME16 | | |
| SK17 | 162 | | EN17 | | | SM17 | 182 | | DM17 | | | ME17 | | |
| SK18 | 158 | | EN18 | 216 | | SM18 | | | DM18 | | | ME18 | | |
| SK19 | | | EN19 | 237 | | SM19 | | | DM19 | | | ME19 | | |
| SK20 | 199 | | EN20 | 241 | | SM20 | | | DM20 | | | ME20 | | |
| SK21 | | | EN21 | | | SM21 | | | DM21 | | | ME21 | | |
| SK22 | | | EN22 | 187 | | SM22 | 156 | | DM22 | | | ME22 | | |
| SK23 | | | EN23 | 217 | | SM23 | | | DM23 | | | ME23 | | |
| SK24 | | | EN24 | | | SM24 | | | DM24 | | | ME24 | | |
| SK25 | 240 | | EN25 | | | SM25 | 166 | | DM25 | | | ME25 | | |
| SK26 | 163 | | EN26 | 140 | | SM26 | | | DM26 | | | ME26 | | |
| SK27 | | | EN27 | 199 | | SM27 | 177 | | DM27 | | | ME27 | | |
| SK28 | 246 | | EN28 | | | SM28 | 150 | | DM28 | | | ME28 | | |
| SK29 | | | EN29 | | | SM29 | | | DM29 | | | ME29 | | |
| SK30 | 148 | | EN30 | | | SM30 | | | DM30 | | | ME30 | | |
| SK31 | 164 | | EN31 | 141 | | SM31 | | | DM31 | | | ME31 | | |
| SK32 | 165 | | EN32 | | | SM32 | | | DM32 | | | ME32 | | |
| SK33 | | | EN33 | | | SM33 | 183 | | DM33 | | | ME33 | | |
| SK34 | | | EN34 | | | SM34 | | | DM34 | | | ME34 | | |
| SK35 | | | EN35 | | | SM35 | | | DM35 | | | ME35 | | |
| SK36 | | | EN36 | | | SM37 | | | DM36 | | | ME36 | | |
| SK37 | | | EN37 | | | SM38 | | | DM37 | | | ME37 | | |
| SK38 | | | EN38 | 202 | | SM39 | | | | | | ME38 | | |
| SK39 | | | EN39 | | | SM40 | | | LS1 | | EpiLife | ME39 | | |
| SK40 | 214 | | EN40 | | | SM41 | | | LS2 | | LSGS | ME40 | | |
| SK41 | | | EN41 | | | SM42 | 149 | | LS3 | | | ME41 | | |
| SK42 | | | EN42 | 175 | | SM43 | | | LS4 | | | ME42 | | |
| SK43 | 147 | | EN43 | 251 | | SM44 | 201 | | | | | ME43 | | |
| SK44 | 204 | | EN44 | 253 | | SM45 | | | | | | | | |
| SK45 | | | EN45 | | | SM46 | | | | | | | | |
| SK46 | 137 | | EN46 | | | SM47 | | | | | | | | |
| SK47 | 138 | | EN47 | 176 | | SM48 | | | | | | | | |
| SK48 | | | EN48 | | | SM49 | 151 | | | | | | | |
| SK49 | 224 | | EN49 | | | SM50 | | | | | | | | |
| SK50 | 159 | | EN50 | 254 | | SM51 | 248 | | | | | | | |
| SK51 | | | EN51 | 220 | | SM52 | | | | | | | | |
| SK52 | 146 | | EN52 | | | | | | | | | | | |
| SK53 | 169 | | EN53 | | | | | | | | | | | |
| SK54 | 160 | | EN54 | | | | | | | | | | | |
| SK55 | | | EN55 | 185 | | | | | | | | | | |
| SK56 | | | | | | | | | | | | | | |
| SK57 | 205 | | | | | | | | | | | | | |
| SK58 | 188 | | | | | | | | | | | | | |
| SK59 | | | | | | | | | | | | | | |
| SK60 | 192 | | | | | | | | | | | | | |
| SK61 | 181 | | | | | | | | | | | | | |

The cell line SK17 (ACTC162) derived in this example displays both cardiac and neuroectodermal (neural crest) and neuroendocrine markers of cardiac neural crest. While the embryological origin of the human heart conduction fibers has been a matter of dispute and uncertainty, the clonal cell line SK17 displays markers, some of which are characteristic of myocardial progenitor cells and some which are evidence of cells of neural crest origin, including: CEACAM1 (Accession number NM_{—}001712.2), ACTC (Accession number NM_{—}005159.2), MYBPH (Accession number NM_{—}004997.1), MYL4 (Accession number NM_{—}002476.2), FABP3 (Accession number NM_{—}004102.2), FABP4 (Accession number NM_{—}001442.1), MYH3 (Accession number NM_{—}002470.1), MYL1 (Accession number NM_{—}079422.1), TNNT2 (Accession number NM_{—}000364.1), TNNC1 (Accession number NM_{—}003280.1), MYH7 (Accession number NM_{—}000257.1), KBTBD10 (Accession number NM_{—}006063.2), CASQ2 (Accession number NM_{—}001232.1), HOXA5 (Accession number NM_{—}019102.2), SST (Accession number NM_{—}001048.2M), SLN (Accession number NM_{—}003063.1), MYOD1 (Accession number NM_{—}002478.3), PCDH7 (Accession number NM_{—}032457.1), CDH2 (Accession number NM_{—}001792.2), CDH15 (Accession number NM_{—}004933.2), TMEM16C (Accession number NM_{—}031418.1), and PCSK1 (Accession number NM_{—}000439.3). SK17 does not express some markers expected of neural crest-derived cells such as BARX1 (Accession number NM_{—}021570.2) and SOX10 (Accession number NM_{—}006941.3). Some markers similar to cells of neuroectodermal origin are LSAMP (Accession number NM_{—}002338.2), SOSTDC1 (Accession number NM_{—}015464.1), SLIT2 (Accession number NM_{—}004787.1), NEF3 (Accession number NM_{—}005382.1), MEIS1 (Accession number NM_{—}002398.2), FOXG1B (Accession number NM_{—}005249.3), and SILV (Accession number NM_{—}006928.3). SK17 cells or cells closely related to SK17 cells may be purified from heterogeneous mixtures of cells, such as hES-derived, hED-derived, hEC-derived, hEG-derived, parthenogentic embryo-derived, heterogeneous mixtures of cells resulting from the in vitro reprogramming of somatic cells as described herein or heterogeneous mixtures of cells derived by directly differentiating from blastomere, morula, ICM cell or other embryo derived cells or from any heterogenous mixtures using cell surface antigens, such as selecting the cells by affinity purification techniques, immunoselection or cell sorting techniques as described herein targeting the antigens CD66A (CEACAM1; accession number NM_{—}001712.2), CD213A2 (IL13RA2; Accession number NM_{—}000640.2); CDw218A (IL18R1; NM_{—}003855.2), CD225 (IFITM1; Accession number NM_{—}003641.2), CD317 (BST2; NM_{—}004335.2), CD9, CD141, CD13, CD26, CD105, CD106, CD124, CDw218, CD317 and CDw325 (CDH2; Accession number NM_{—}001792.2), as these are the antigens that are expected to be expressed on SK17 cells. Contaminating cells can be removed utilizing antigens expressed by these cells at relatively low levels such as the two antigens, CD141 (THBD; NM_{—}000361.2) and CD9 (CD9; NM_{—}001769.2).

Purification of SK17 cells or cells closely related to SK17 cells from heterogeneous mixtures of cells derived from pluripotent cells may be accomplished by immunoaffinity-based cell selection methods, e.g., with magnetic beads or FACS, using a single antibody or an antibody cocktail to select antigen positive cells from antigen negative cells, or bright from dull cells (referring to the level of fluorescence in cells that have reacted with antibodies to a cell surface antigen, wherein the antibody is tagged directly or indirectly [e.g., via a secondary antibody or biotin-avidin link] with a fluorescent probe or fluorophore), in either a positive or negative direction (typically once positively). The antibody or antibodies may be targeted to one or more of the following antigens that may be expressed on the surface of SK17 cells or cells related to SK17: CD66A (CEACAM1; accession number NM_{—}001712.2), CD213A2 (IL13RA2; Accession number NM_{—}000640.2), CDw218A (IL18R1; NM_{—}003855.2), CD225 (IFITM1; Accession number NM_{—}003641.2), CD317 (BST2; NM_{—}004335.2), CD9, CD141, CD13, CD26, CD105, CD106, CD124, CDw218, CD317 and CDw325 (CDH2; Accession number NM_{—}001792.2). FACS offers much greater capability for multiparameter sorting of these cell subpopulations using numerous antibodies, even when there is overlapping expression of individual markers. An antibody specific for CD66a alone may be sufficient to purify SK17 cells, or cells closely related to SK17 cells by immunoaffinity-based selection or FACS. Alternatively or in addition, these cells can be can be identified and sorted by FACS from other cell types according to qualitative or quantitative differences in antigen expression among the different cell types. Methods of labeling cells using antibodies or antibody cocktails tagged with fluorescent probes or fluorophores, followed by gating and sorting the cell populations according to the amount of fluorescence of different antigens, are widely practiced in the art.

The SK17 cells also have use in vitro in cell-based drug discovery in screening for bioactive agents on myocardium. The SK17 cells can be in the relatively undifferentiated state they are in when cultured in the medium described, or by allowing the cells to become confluent for one or more weeks alone or on vascular endothelial feeder cells, the cells differentiate into terminally differentiated beating myocardium that can be the substrate for drug screening.

The SK17 or analogous myocardial progenitors can be combined with conjugated antibodies such that one antibody recognizes an antigen on the surface of the myocardial progenitors and the other antibody recognizes antigens present in the target tissue such as the heart. Antigens on the surface of the myocardial cells can be by way of nonlimiting example any of those mentioned above with respect to SK17. Antigens specific to the heart include by way of nonlimiting example HCN4 ion channel present in the SA node. Such antibody tagged cells are useful in targeting the cells to the site of interest and for causing the cells to be retained at the injection site.

The cell line SK5 (ACTC157) derived in this example also displays both cardiac and neuroectodermal (neural crest) markers of cardiac neural crest, but markers distinct from SK17, including: ACTC (Accession number NM_{—}005159.2), MYBPH (Accession number NM_{—}004997.1), MYL4 (Accession number NM_{—}002476.2), FABP3 (Accession number NM_{—}004102.2), MYH3 (Accession number NM_{—}002470.1), MYL1 (Accession number NM_{—}079422.1), TNNC1 (Accession number NM_{—}003280.1), KBTBD10 (Accession number NM_{—}006063.2), HOXA5 (Accession number NM_{—}019102.2), MYOD1 (Accession number NM_{—}002478.3), CDH2 (Accession number NM_{—}001792.2), CDH15 (Accession number NM_{—}004933.2), C7 (Accession number NM_{—}000587.2), and TNA (Accession number NM_{—}003278.1). SK5 does not express MYOG (Accession number NM_{—}002479.2) and does not express some markers expected of neural crest-derived cells such as SOX10 (Accession number NM_{—}006941.3) but does express BARX1 (Accession number NM_{—}021570.2), FOXG1B (Accession number NM_{—}005249.3), HOXA2 (Accession number NM_{—}006735.3), and MEIS1 (Accession number NM_{—}002398.2) reported to correlate with neural crest. The cells may be purified from heterogeneous mixtures of cells, such as hES, hED, hEC, hEG, pathenogentic embryo-derived, heterogeneous mixtures of cells resulting from the in vitro reprogramming of somatic cells as described herein using cell surface antigens, such as selecting the cells by affinity purification techniques as described herein targeting the antigens CD42c (GP1BB; accession number NM_{—}000407.3), CD225 (IFITM1; Accession number NM_{—}003641.2), and CDW218A (IL18R1; Accession number NM_{—}003855.2) or other CD antigens differentially expressed in these cells.

The cell lines SK17 (ACTC162) or SK5 (ACTC157) or equivalent cells clustering cells are easily propagated using the medium in which they were clonally expanded using standard cell culture techniques, such as the use of cell culture flasks, roller bottles, beads, tubes, or other standard culture systems and normal trypsinization. In this case, the medium is PromoCell Skeletal Muscle Medium (Cat# C-23260 with Supplementary growth factors (PromoCell Cat#C-39360) (Table I condition 1112). Alternatively, Promocell skeletal muscle medium can be replaced with the basal medium MCDB120 supplemented with 5% Fetal Calf Serum, Fetuin 50 ug/ml, Basic Fibroblast Growth Factor 1 ng/ml, Epidermal Growth Factor 10 ng/ml, Insulin 10 ug/ml, and Dexamethasone 0.4 ug/ml all shown at their final concentrations.

The cell lines SK17 (ACTC162) or SK5 (ACTC157) or equivalent cells clustering cells are useful when injected into myocardium via a syringe, catheter, or other means of introduction known in the art for restoring the functional cells to the heart. SK17 is useful for restoring the conduction fiber system including sinoatrial node, AV node, AVBB, and purkinje fibers following damage to the conduction system by infaction or inherited disease. SK17 also produces PTN, BMP5, and PDGFD useful in inducing angiogenesis in and regenerating infracted heart and are useful in the treatment of chronic ischemic disease of the heart. In addition, they are useful in regenerating heart muscle, the SA node, the AVB, AV node, and purkinje fibers, following myocardial infarction, idiopathic heart disease, or heart failure. SK5, because of its expression of high levels of TNA, is useful in restoring myocardium in the regions of ligament attachment of other regions of the heart wall where high tensile strength is desirable.

The cell lines EN7 and EN13 (ACTC174) show properties of cranial neural crest in that they express relatively high levels of HOXA2, HOXB2, NEF3 (Accession number NM_{—}005382.1), CGI-38 (Accession Number NM_{—}015964.1), NP25 (Accession number NM_{—}013259.1A), and ENO2 (NM_{—}001975.2), showing their normal migration through the second branchial arch and potential for differentiation into bones such as the lesser horn of the hyoid bone, the stylohyoid ligament, the styloid process, and the stapes, muscles such as the buccinator, platysma, stapedius, stylohyoid, and the posterior belly of the digastric, and cranial nerve VII and are useful in regenerating numerous tissues including the dermis of the face and neck with a prenatal pattern of gene expression useful in the scarless regeneration of skin as described herein.

### Example 34

### Derivation of Initial Heterogeneity in Skeletal Muscle Medium

In another series herein designated series EB5, H9 human embryonic stem (hES) cells were routinely cultured in hES medium (KO-DMEM, 1× nonessential amino acids, 1× Glutamax-1, 55 uM beta-mercaptoethanol, 10% Serum Replacement, 10% Plasmanate, 10 ng/ml LIF, 4 ng/ml bFGF, and penicillin/streptomycin) and passaged by manual dissection. Except where indicated, all tissue culture plastic wares were coated with 0.1% gelatin. Before processing cells to make embryoid bodies, H9 hES cells were cultured for 2 days in Skeletal Muscle Cell Growth Medium supplemented with penicillin/streptomycin. To process cells to make embryoid bodies, H9 hES cells were harvested by manual dissection of individual colonies, the cell-clump suspension was replated into non-coated 10 cm plastic bacterial Petri dishes in PromoCell Skeletal Muscle Cell Growth Medium with penicillin/streptomycin and cultured for 4 days at 37 deg C. (10% CO2, 5% O2). Unattached bulk embryoid bodies were harvested by aspirating growth medium and attached cells were harvested by trypsinization and pooled with unattached bulk embryoid bodies. Cells were concentrated by centrifugation and replated at a density of approximately 1000 to 2000 cells/15 cm tissue culture dish in their respective medium. After approximately two weeks of growth, colonies were picked from cells grown in each medium. Colonies were serially grown in 24 well, 12 well, 6 well tissue culture plates, T25, T75, T150 flasks, and 2 liter Roller Bottles (850 cm² surface area) before freezing and storing in liquid nitrogen. Cell morphologies and cell growth were monitored by phase contract microscopy and recorded by photomicroscopy. Cells were cultured in 6 well tissue culture plates or 6 cm tissue culture Petri dishes to harvest RNA for gene expression analysis using the Illumina human sentra-6 platform.

EB(5) Exp.

| Line Name | ACTC No. | Medium |
|---|---|---|
| MW1 | 242 | Skeletal Medium |
| MW2 | 189 | |
| MW3 | | |
| MW4 | | |
| MW5 | | |
| MW6 | 193 | |
| MW7 | | |
| MW8 | | |

TOTAL COLONIES EB(5) = 8

### Example 35

### Derivation of Initial Heterogeneity in 10% Plasmanate in Hanging Drop Suspension

In this series designated series EB4, H9 human embryonic stem (hES) cells were routinely cultured in hES medium (KO-DMEM, 1× nonessential amino acids, 1× Glutamax-1, 55 uM beta-mercaptoethanol, 10% Serum Replacement, 10% Plasmanate, 10 ng/ml LIF, 4 ng/ml bFGF, and penicillin/streptomycin) and passaged by manual dissection. Except where indicated, all tissue culture plastic wares were coated with 0.1% gelatin. Before processing cells to make embroid bodies, 119 hES cells were cultured for 2 days with medium containing KO-DMEM, 1× nonessential amino acids, 1× Glutamax-1, 55 uM beta-mercaptoethanol, 10% Plasmanate, with penicillin/streptomycin. To process cells to make embryoid bodies, 119 hES cells were harvested by manual dissection of individual colonies. The cell-clump suspension was dispersed into 35 hanging-drops (15 ul/drop in medium containing KO-DMEM, 1× nonessential amino acids, 1× Glutamax-1, 55 uM beta-mercaptoethanol, 10% Plasmanate, with penicillin/streptomycin) on the non-coated lid of a 10 cm plastic bacterial Petri dish. After 4 days of culture at 37° C. (10% CO2, 5% O2), embryoid bodies were collected by centrifugation in 10 ml phosphate buffered saline. Harvested embryoid bodies were dispensed to 6 well tissue culture dishes, and cultured in PromoCell Endothelial Cell Growth medium, PromoCell Skeletal Muscle. Cell Growth medium, PromoCell Smooth Muscle Cell Growth medium, Stem Cell Technology Mesenchymal medium, EpiLife LSGS medium, or DMEM containing 20% fetal bovine serum (FBS) (all supplemented with penicillin/streptomycin). Only cells cultured with first three media continued to grow and were subsequently cultured in their respective PromoCell Endothelial Cell Growth medium, PromoCell Skeletal Muscle Cell Growth medium, or PromoCell Smooth Muscle Cell Growth medium. Cells were serially grown in 12 well, 6 well, and 10 cm tissue culture dishes and finally replated at a density of 1000 cells/15 cm tissue culture dish in their respective medium. After approximately two weeks of growth, a total of 11 colonies were picked from, cells grown in each medium, for a total of 33 colonies. Colonies were serially grown in 24 well, 12 well, 6 well tissue culture plates, T25, T75, T150 flasks, and 2 liter Roller Bottles before freezing and storage in liquid nitrogen. Cells were cultured in 6 well tissue culture plates or 6 cm tissue culture Petri dishes to harvest RNA for gene expression analysis using the Illumina human sentra-6 platform. The cell line Z11 was isolated from embryoid bodies cultured in Smooth Muscle Cell Growth medium.

| Line | ACTC | | Line | ACTC | | Line | | |
|---|---|---|---|---|---|---|---|---|
| Name | No. | Medium | Name | No. | Medium | Name | ACTC No. | Medium |
| Q1 | | Skeletal | W1 | | PromoCell | Z1 | | Smooth |
| Q2 | | Muscle | W2 | | Endothelial | Z2 | 255 | Muscle |
| Q3 | | Medium | W3 | | | Z3 | 244 | Media |
| Q4 | | | W4 | | | Z4 | | |
| Q5 | | | W5 | | | Z5 | | |
| Q6 | | | W6 | | | Z6 | 195 | |
| Q7 | 235 | | W7 | 228 | | Z7 | 200 | |
| Q8 | 239 | | W8 | 245 | | Z8 | 213 | |
| Q9 | | | W9 | | | Z9 | | |
| Q10 | | | W10 | 196 | | Z10 | | |
| Q11 | | | W11 | 197 | | Z11 | 194 | |

TOTAl COLONIES EB(4) = 33

### Example 36

### Derivation of Initial Heterogeneity in Neural Basal Medium

In this series designated series EB1, H9 human embryonic stem (hES) cells were routinely cultured in hES medium (KO-DMEM, 1× nonessential amino acids, 1× Glutamax-1, 55 uM beta-mercaptoethanol, 10% Serum Replacement, 10% Plasmanate, 10 ng/ml LIF, 4 ng/ml bFGF, and 1% penicillin/streptomycin and passaged by manual dissection. Except where indicated, all tissue culture plastic wares were coated with 0.1% gelatin. Before processing cells to make embryoid bodies, H9 hES cells were cultured for 2 days in Neural Basal N2 medium supplemented with penicillin/streptomycin. To process cells to make embryoid bodies, H9 hES cells were harvested by trypsinization and replated into non-coated 10 cm plastic bacterial Petri dishes in Neural Basal N2 medium with penicillin/streptomycin and cultured for 11 days at 37 deg C. (10% CO2, 5% O2). Unattached bulk embryoid bodies were harvested by aspirating growth medium and attached cells were harvested by trypsinization and pooled with unattached bulk embryoid bodies. Cells were concentrated by centrifugation and plated into 6 well tissue culture dish in DMEM containing 20% FBS. Cells were grown to confluence and finally replated at a density of approximately 1000 to 2000 cells/15 cm tissue culture dish in either DMEM 20% PBS or Stem Cell Technology Mesenchymal medium supplemented with penicillin/streptomycin. After approximately two weeks of growth, colonies were picked from cells grown in each medium. Colonies were serially grown in 24 well, 12 well, 6 well tissue culture plates, T25, T75, T150 flasks, and 2 liter Roller Bottles (850 cm² surface area) before freezing and storage in liquid nitrogen. Cell morphologies and cell growth were monitored by phase contract microscopy and recorded by photomicroscopy. Cells were cultured in 6 well tissue culture plates or 6 cm tissue culture Petri dishes to harvest RNA for gene expression analysis using the Illumina human sentra-6 platform.

EB(1) Exp.

| Line | ACTC | | | ACTC | |
|---|---|---|---|---|---|
| Name | No. | Medium | | No. | Medium |
| T1 | | DMEM 20% | U1 | | Mesenchymal |
| T2 | | Fetal | U2 | | Media |
| T3 | | Bovine Serum | U3 | | |
| T4 | | | U4 | | |
| T5 | | | U5 | | |
| T6 | | | U6 | | |
| T7 | 186 | | U7 | 186 | |
| T8 | | | U8 | | |
| T9 | | | U9 | | |
| T10 | | | U10 | | |
| T11 | | | U11 | | |
| T12 | | | U12 | | |
| T13 | | | U13 | | |
| T14 | 211 | | U14 | 211 | |
| T15 | | | U15 | | |
| T16 | | | U16 | | |
| T17 | | | U17 | | |
| T18 | | | U18 | | |
| T19 | | | U19 | | |
| T20 | 231 | | U20 | 231 | |
| T21 | | | U21 | | |
| T22 | | | U22 | | |
| T23 | | | U23 | | |
| T24 | | | U24 | | |
| T25 | | | U25 | | |
| T26 | | | U26 | | |
| T27 | | | U27 | | |
| T28 | | | U28 | | |
| T29 | | | U29 | | |
| T30 | | | U30 | | |
| T31 | | | U31 | 236 | |
| T32 | | | U32 | | |
| T33 | | | U33 | | |
| T34 | | | | | |
| T35 | | | | | |
| T36 | 198 | | | | |
| T37 | | | | | |
| T38 | | | | | |
| T39 | | | | | |
| T40 | | | | | |
| T41 | | | | | |
| T42 | 210 | | | | |
| T43 | 120 | | | | |
| T44 | 106 | | | | |
| T45 | | | | | |
| T46 | | | | | |
| T47 | | | | | |
| T48 | | | | | |

TOTAL COLONIES EB(1) = 81

### Example 37

### Derivation of Initial Heterogeneity in 10% FBS DMEM the Clonal Propagation in a Variety of Culture Media

In this series designated series C5, a frozen ampule of approximately 1×10⁶ heterogeneous cells previously remaining from the experiment described in Example 17 and derived from the hES cell line ACT3 differentiated for 7 days was thawed and cultured for five days in 10% FBS DMEM, then trypsinized, counted and 2,000 cells were plated onto gelatinized 15 cm plates in the following media: DMEM 5% FBS (Table I conditions 455 and 1103), DMEM 10% FBS (Table I conditions 456 and 1103), DMEM 20% FBS (Table I conditions 457 and 1103), PromoCell Skeletal Muscle Cell Growth medium (Table I condition 1112), PromoCell Smooth Muscle Cell Growth medium (Table I condition 1113), PromoCell Endothelial Cell Growth medium (Table I condition 1110), Stem Cell Technology Mesenchymal medium (Table I condition 1114), or EpiLife LSGS medium (Table I condition 1109), each supplemented with penicillin/streptomycin (Table I conditions 1127 and 1128). The cell clones picked and the cell lines isolated capable of long-term propagation are shown below.

Experiment C5 (300 colonies picked)

| Line | | ACTC | Line | | ACTC | Line | | ACTC | Line | | ACTC |
|---|---|---|---|---|---|---|---|---|---|---|---|
| Name | Media | No. | Name | Media | No. | Name | Media | No. | Name | Media | No. |
| E9 | DMEM; 20% FBS | | E1 | DMEM; 10% FBS | | E126 | DMEM; 5% FBS | | G1 | Skeletal Muscle | |
| E10 | | 121 | E2 | | | E127 | | | G2 | | |
| E11 | | | E3 | | 88 | E128 | | | G3 | | |
| E12 | | | E4 | | | E129 | | | G4 | | |
| E13 | | | E5 | | | E130 | | | G5 | | |
| E14 | | | E6 | | | E131 | | | G6 | | 134 |
| E15 | | 98 | E7 | | | E132 | | | G7 | | |
| E16 | | | E8 | | 96 | E133 | | | G8 | | |
| E17 | | 94 | E20 | | | E134 | | | G9 | | |
| E18 | | | E21 | | | E135 | | | G10 | | |
| E19 | | 105 | E22 | | | E136 | | | G11 | | |
| E30 | | 179 | E23 | | | E137 | | | G12 | | |
| E31 | | | E24 | | | E138 | | | G13 | | |
| E32 | | | E25 | | | E139 | | | G14 | | |
| E33 | | 114 | E26 | | 80 | E140 | | | G15 | | |
| E34 | | 85 | E27 | | | E141 | | | F1 | Smooth Muscle | |
| E35 | | 113 | E28 | | | E142 | | | F2 | | |
| E36 | | | E29 | | | E143 | | | F3 | | |
| E37 | | | E77 | | | E144 | | | F4 | | |
| E38 | | | E78 | | | E145 | | | F5 | | |
| E39 | | | E79 | | | E146 | | | F6 | | |
| E40 | | 95 | E80 | | 115 | E147 | | | F7 | | |
| E41 | | | E81 | | | E148 | | | F8 | | |
| E42 | | | E82 | | | E149 | | | F9 | | |
| E43 | | | E83 | | | E150 | | | F10 | | |
| E44 | | 170 | E84 | | 116 | E151 | | | F11 | | |
| E45 | | 99 | E85 | | | E152 | | | F12 | | |
| E46 | | | E86 | | | E154 | | | F13 | | |
| E47 | | | E87 | | | E155 | | | F14 | | |
| E48 | | | E88 | | | E156 | | | F15 | | |
| E49 | | | E89 | | | E157 | | | F16 | | |
| E50 | | 178 | E90 | | | E158 | | | M1 | Mesenchymal | |
| E51 | | 86 | E91 | | | E159 | | | M2 | | |
| E52 | | | E92 | | | E160 | | | M3 | | |
| E53 | | 222 | E93 | | 90 | E161 | | | M4 | | |
| E54 | | | E94 | | | E162 | | | M5 | | |
| E55 | | | E95 | | | E163 | | 132 | M6 | | |
| E56 | | | E96 | | | E164 | | 209 | M7 | | |
| E57 | | 91 | E97 | | | E165 | | | M8 | | |
| E58 | | | E98 | | | E166 | | | M9 | | |
| E59 | | | E99 | | | E167 | | | M10 | | 103 |
| E60 | | | E100 | | | E168 | | | M11 | | 233 |
| E61 | | 107 | E101 | | | E169 | | 208 | M12 | | |
| E62 | | | E102 | | | E170 | | | M13 | | 104 |
| E63 | | | E103 | | | | | | M14 | | |
| E64 | | | E104 | | | | | | M15 | | |
| E65 | | 171 | E105 | | | | | | M16 | | |
| E66 | | | E106 | | | | | | M17 | | |
| E67 | | 97 | E107 | | | | | | M18 | | |
| E68 | | 207 | E108 | | 112 | | | | J1 | EpiLife + | |
| E69 | | 101 | E109 | | 117 | | | | J2 | LSGS | 206 |
| E70 | | | E110 | | | | | | J3 | | |
| E71 | | 81 | E111 | | 223 | | | | J4 | | 161 |
| E72 | | 100 | E112 | | | | | | J5 | | |
| E73 | | 89 | E113 | | | | | | J6 | | |
| E74 | | | E114 | | | | | | J7 | | |
| E75 | | 102 | E115 | | | | | | J8 | | 119 |
| E76 | | 93 | E116 | | | | | | J9 | | |
| | | | E117 | | | | | | J10 | | |
| | | | E118 | | | | | | J11 | | |
| | | | E119 | | | | | | J12 | | |
| | | | E120 | | | | | | J13 | | 172 |
| | | | E121 | | | | | | J14 | | |
| | | | E122 | | 180 | | | | J15 | | |
| | | | E123 | | | | | | J16 | | 136 |
| | | | E124 | | | | | | J17 | | |
| | | | | | | | | | J18 | | |

### Example 38

### Derivation of Initial Heterogeneity in 10% FBS DMEM, Mesencult, and EpiLife LSGS Media

In this series designated series C4, hES cell line H9 was subcultured as previously described in Example 17, then after three days of culture after passage, the media in the six well plate containing the colonies was aspirated and replaced with either DMEM 10% FBS (Table I, conditions 1103 and 456), Stem Cell Technology Mesenchymal Media (Mesencult) (Table I, condition 1114) or EpiLife LSGS Media (Table I, condition 1105) and culture for 3 days. Embryoid bodies were then prepared in the same media for each cell culture and the enriched heterogeneous culture was propagated clonally, mRNA isolated and analyzed, and the cell lines were cryopreserved as previously described (Example 37) and the resulting cultures are shown in the table below.

Experiment C4

| Clone | Media | ACTC No. |
|---|---|---|
| 10% 1 | DMEM + 10% FBS | |
| 10% 2 | | |
| 10% 3 | | |
| 10% 4 | | 87 |
| 10% 5 | | |
| 10% 6 | | |
| 10% 7 | | |
| 10% 8 | | |
| ELSR 1 | LSGS | |
| ELSR 2 | | 167 |
| ELSR 3 | | |
| ELSR 4 | | |
| ELSR 5 | | 135 |
| ELSR 6 | | |
| ELSR 7 | | |
| ELSR 8 | | |
| ELSR 9 | | |
| ELSR 10 | | 152 |
| ELSR 11 | | |
| ELSR 12 | | 131 |
| ELSR 13 | | 243 |
| ELSR 14 | | 92 |
| ELSR 15 | | |
| ELSR 16 | | |
| ELSR 17 | | |
| ELSR 18 | | 108 |
| ELSG 1 | LSGS | 130 |
| ELSG 2 | | |
| ELSG 3 | | |
| ELSG 4 | | |
| ELSG 5 | | 135 |
| ELSG 6 | | 118 |
| ELSG 7 | | |
| ELSG 8 | | 238 |
| ELSG 9 | | |
| ELSG 10 | | |
| ELSG 11 | | |
| ELSG 12 | | |
| Mesen 1 | Mesen. | |
| Mesen 2 | | |
| Mesen 3 | | 133 |

TOTAL COLONIES EXPT. C4 = 42

### Example 39

### Derivation of Myocardial Progenitors Similar to SK17 (ACTC 162) from hED Cells

Myocardial progenitors may be generated from hED cells directly differentiated from human preimplantaion embryos without the intermediate step of generating human ES cell lines. Human pluripotent cells from preimplantation embryos, in this example, from a human blastocyst, are obtained by gently tearing the trophectoderm of the blastocyst and plating the opened embryo onto collagen coated six well plates in standard human embryo culture medium. The initial differentiation and generation of heterogeneity is performed in 5% FBS containing DMEM (Table I conditions 455 and 1103). Except where indicated, all tissue culture plastic wares were coated with 0.1% gelatin. Before processing cells to make embryoid bodies, the opened blastocysts are cultured for 5 days in DMEM 5% fetal bovine serum (FBS) supplemented with penicillin/streptomycin. To process cells to make embryoid bodies, the attached cells are harvested by manual dissection of the attached colonies, the cell-clump suspension is replated into a non-coated 10 cm plastic bacterial Petri dish in DMEM 5% PBS and cultured for 7 days at 37 deg C. (10% CO2, 5% O2). Unattached bulk embryoid bodies are harvested by aspirating growth medium and attached cells were harvested by trypsinization and pooled with unattached bulk embryoid bodies. Cells are concentrated by centrifugation and plated for the second step of clonal isolation into 6 well tissue culture dishes in PromoCell Skeletal Muscle Cell Growth medium (Table I condition 1112) supplemented with penicillin/streptomycin (Table I conditions 1127 and 1128). Cells are serially grown in 6 well, and 10 cm tissue culture dishes and finally replated at a density of approximately 1000 to 2000 cells/15 cm tissue culture dish in the same media with penicillin/streptomycin. Cells are plated at high densities of 2000, 5000 and 10,000 cells/15 cm tissue culture dish. After approximately two weeks of growth, colonies are picked. Colonies are serially grown in 24 well, 12 well, 6 well tissue culture plates, T25, T75, T150 flasks, and 2 liter Roller Bottles (850 cm2 surface area) before freezing and storage in liquid nitrogen. Cell morphologies and cell growth are monitored by phase contract microscopy and recorded by photomicroscopy. Cells are cultured in 6 well tissue culture plates or 6 cm tissue culture Petri dishes to harvest RNA for gene expression analysis using the Illumina human sentra-6 platform. Colonies with a pattern of gene expression similar to SK17 can be obtained by using the enrichment step described herein after selecting cells with the cell surface antigens of SK 17. For example, after the initial 5 days of culture of the disrupted blastocyst, and the subsequent 7 days of culture in Promocell Skeletal Muscle Medium, the cells can be detached using a light trypsin treatment, incubated in suspension to repair cell surface antigens, and subjected to flow cytometry using antibodies to the following antigens: CD66A (CEACAM1; accession number NM_{—}001712.2), CD213A2 (IL13RA2; Accession number NM_{—}000640.2), CDw218A (IL18R1; NM_{—}003855.2), CD225 (IFITM1; Accession number NM_{—}003641.2), CD317 (BST2; NM_{—}004335.2), and CDw325 (CDH2; Accession number NM_{—}001792.2). Contaminating cells can be removed utilizing antigens expressed by these cells at relatively low levels such as the two antigens, CD141 (THBD; NM_{—}000361.2) and CD9 (CD9; NM_{—}001769.2). The resulting selected cells can then be plated at clonal densities as described above to obtain an increased frequency of colonies similar to SK17.

Cells similar in gene expression to cell line SKI 7 derived herein display both cardiac and neuroectodermal (neural crest) and neuroendocrine markers of cardiac neural crest. While the embryological origin of the human heart conduction fibers has been a matter of dispute and uncertainty, the clonal cell line SK17 shows the markers, including both markers characteristic of myocardial cells and neuronal cells including CEACAM1, ACTC, MYBPH, MYL4, FABP3, FABP4, MYH3, MYL1, TNNT2, TNNC1, MYH7, KBTBD10, CASQ2, HOXA5, CLDN5, SST, SLN, MYOD1, PCDH7, CDH2, CDH15, TMEM16C, and PCSK1. Some markers similar to cells of neuroectodermal origin are LSAMP, SOSTDC1, SLIT2, NEF3, MEIS1, and SILV. This cell type may be identified in the hED cell colonies by a combination of these markers at levels when compared housekeeping genes such as ADPRT or GAPD or by correlation by hierarchical clustering with the SK17 cell line as described herein. hED cell lines with a gene expression profile similar to the cell line SK17 are useful when injected into myocardium via a syringe, catheter, or other means of introduction known in the art for restoring the conduction fiber system including sinoatrial node, AV node, AVBB, and purkinje fibers following damage to the conduction system by infaction or inherited disease. They also produce PTN, BMP5, and PDGFD useful in inducing angiogenesis in and regenerating infracted heart and are useful in the treatment of chronic ischemic disease of the heart. In addition, they are useful in regenerating heart muscle, the SA node, the AVB, AV node, and purkinje fibers, following myocardial infarction, idiopathic heart disease, or heart failure.

### Example 40

### Initial Heterogeneity Generated in Diverse Temporal Combinations of Differentiation Conditions

Human embryonic stem (hES) cell line H-9 was cultured as described according to the methods of this invention and then passage 48 cells were plated in a standard 6 well tissue culture plate on a feeder layer of mouse embryonic fibroblasts and allowed to grow for 9 days to confluence. The hES cell growth medium was then replaced by 6 specialized media and the hES cells were allowed to differentiate for 3 days. The six media were: DMEM 10% FBS (Table I, conditions 456 and 1103), PromoCell Skeletal Muscle Cell Growth medium (Table I, condition 1112), PromoCell Endothelial Cell Growth medium (Table I, condition 1110), or EpiLife LSGS medium (Table I, condition 1109), Gibco Neurobasal Medium B27 (Table I, condition 1106), and PromoCell Airway Epithelial Medium (Table I, condition 1104) each supplemented with penicillin/streptomycin (Table I conditions 1127 and 1128).

The cells were trypsinized (0.05% trypsin) and transferred to Corning 24-well, ultra low attachment tissue culture plates containing 12 specialized media (see Table XIII) to form embryoid bodies and for further differentiation. One well of differentiated hES cells (6 well plate) was equally divided between 2 wells (24 well plate) containing 2 different media and allowed to form embryoid bodies. For example, well number 1 of the original 6 well plate in which the hES cells were allowed to differentiate in Airway Eiphelial Medium for 3 days were trypsinized and half the cells are placed in a well of an ultra low attachment plate containing the same Airway Epithelial Medium and the other half of the cells transferred to a second well of the ultra low attachment plate containing Epi-Life LSGS Medium.

TABLE XIII
EMBRYOID BODY MEDIA

| | Differentiation | Embryoid Body | | | |
|---|---|---|---|---|---|
| hES Cell Well | Medium | Well (Ultra Low | | | |
| (Original 6 Well | (Original 6 Well | Attachment | EMBRYOID | | |
| Plate) | Plate) | Plate) | BODY MEDIA | Manufacturer | Catalog Number |
| Well 1 | Airway Epithelial | 1 | Airway Epithelial | PromoCell | C-21260 |
| | Medium | | Growth Medium | | |
| | | 2 | Epi-Life (LSGS) | Cascade | M-EPIcf/PRF-500 |
| | | | Medium. | | |
| Well 2 | Neurobasal | 3 | Neurobasal | Gibco | 12348-017 |
| | Medium - B27 | | Medium - B27 | | |
| | | 4 | Neurobasal | Gibco | 12348-017 |
| | | | Medium - N2 | | |
| Well 3 | Epi-Life (LKGS) | 5 | HepatoZyme- | Gibco | 17705-021 |
| | Medium. | | SFM | | |
| | | 6 | Epi-Life (HKGS) | Cascade | M-EPIcf/PRF-500 |
| | | | Medium. | | |
| Well 4 | Endothelial Cell | 7 | Endothelial Cell | PromoCell | C-22221 |
| | Medium | | Growth Medium | | |
| | | 8 | Endothelial Cell | Gibco | 11111-044 |
| | | | SFM | | |
| Well 5 | Skeletal Muscle | 9 | Skeletal Muscle | PromoCell | C-23260 |
| | Cell Medium | | Cell Growth | | |
| | | | Medium | | |
| | | 10 | Smooth Muscle | PromoCell | C-22262 |
| | | | Basal Medium | | |
| Well 6 | DMEM + 10% | 11 | DMEM + 20% | Hyclone | SH302285-03 |
| | FBS | | FBS | | |
| | | 12 | Melanocyte | PromoCell | C-24010 |
| | | | Growth Medium | | |

The embryoid bodies were allowed to differentiate for 7-10 days, collected, washed in phosphate buffered saline, dissociated into single cells with trypsin (0.25% trypsin) and the differentiated cells plated out in extra cellular matrix coated 15 cm plates (Table XIV). The differentiated cells are allowed to proliferate for 7-20 days and the resulting colonies are cloned and plated in 24 well plates containing the same medium and extra cellular matrix from which they were derived. The cloned colonies are expanded to obtain a stock of cells and the cell line stocks are cryopreserved.

TABLE XIV
EXTRACELLULAR MATRIX & GROWTH MEDIUM

| 15 cm Plate | Selection & Growth Media | Extra Cellular Matrix |
|---|---|---|
| 1 | Airway Epithelial Growth | Gelatin |
| | Medium | |
| 2 | Epi-Life (LSGS) Medium. | Collagen IV |
| 3 | Neurobasal Medium - B27 | Poly-lysine - BioCoat |
| 4 | Neurobasal Medium - N2 | Poly-lysine - BioCoat |
| 5 | HepatoZyme-SFM | Collagen IV |
| 6 | Epi-Life (HKGS) Medium. | Collagen IV |
| 7 | Endothelial Cell Growth Medium | Gelatin |
| 8 | Endothelial Cell SFM | Gelatin |
| 9 | Skeletal Muscle Cell Growth | Gelatin |
| | Medium | |
| 10 | Smooth Muscle Basal Medium | Gelatin |
| 11 | DMEM + 20% FBS | Gelatin |
| 12 | Melanocyte Growth Medium | Gelatin |

The cell clones picked were serially passaged into larger culture vessels as previously described. RNA extraction and microarray analysis of gene expression was determined for the cell lines as previously described. Cell lines obtained are shown below:

| Media 1. H-9 hES Cell | | | |
|---|---|---|---|
| Embryoid Body | Media 2. | | |
| Differentiation | Cloning/Expansion | Clone | ACTC |
| Medium□ | Medium□ | Name □ | Number□ |
| PromoCell Endothelial | PromoCell Endothelial | 4-PEND-1 | NA |
| PromoCell Endothelial | PromoCell Endothelial | 4-PEND-2 | NA |
| PromoCell Endothelial | PromoCell Endothelial | 4-PEND-3 | NA |
| PromoCell Endothelial | PromoCell Endothelial | 4-PEND-4 | NA |
| PromoCell Endothelial | PromoCell Endothelial | 4-PEND-5 | NA |
| PromoCell Endothelial | PromoCell Endothelial | 4-PEND-6 | NA |
| PromoCell Endothelial | PromoCell Endothelial | 4-PEND-7 | NA |
| PromoCell Endothelial | PromoCell Endothelial | 4-PEND-8 | NA |
| PromoCell Skeletal | PromoCell Skeletal | 4-SKEL-1 | NA |
| PromoCell Skeletal | PromoCell Skeletal | 4-SKEL-2 | NA |
| PromoCell Skeletal | PromoCell Skeletal | 4-SKEL-3 | NA |
| PromoCell Skeletal | PromoCell Skeletal | 4-SKEL-4 | 126 |
| PromoCell Skeletal | PromoCell Skeletal | 4-SKEL-5 | NA |
| PromoCell Skeletal | PromoCell Skeletal | 4-SKEL-6 | NA |
| PromoCell Skeletal | PromoCell Skeletal | 4-SKEL-7 | NA |
| PromoCell Skeletal | PromoCell Skeletal | 4-SKEL-8 | 110 |
| PromoCell Skeletal | PromoCell Skeletal | 4-SKEL-9 | NA |
| PromoCell Skeletal | PromoCell Skeletal | 4-SKEL-10 | NA |
| PromoCell Skeletal | PromoCell Skeletal | 4-SKEL-11 | NA |
| PromoCell Skeletal | PromoCell Skeletal | 4-SKEL-12 | NA |
| PromoCell Skeletal | PromoCell Skeletal | 4-SKEL-13 | NA |
| PromoCell Skeletal | PromoCell Skeletal | 4-SKEL-14 | NA |
| PromoCell Skeletal | PromoCell Skeletal | 4-SKEL-15 | NA |
| PromoCell Skeletal | PromoCell Skeletal | 4-SKEL-16 | NA |
| PromoCell Skeletal | PromoCell Skeletal | 4-SKEL-17 | NA |
| PromoCell Skeletal | PromoCell Skeletal | 4-SKEL-18 | NA |
| PromoCell Skeletal | PromoCell Skeletal | 4-SKEL-19 | 83 |
| PromoCell Skeletal | PromoCell Skeletal | 4-SKEL-20 | 127 |
| PromoCell Skeletal | PromoCell Skeletal | 4-SKEL-21 | NA |
| DMEM + 10% FBS | DMEM + 20% FBS | 4-D20-1 | NA |
| DMEM + 10% FBS | DMEM + 20% FBS | 4-D20-2 | NA |
| DMEM + 10% FBS | DMEM + 20% FBS | 4-D20-3 | NA |
| DMEM + 10% FBS | DMEM + 20% FBS | 4-D20-4 | NA |
| DMEM + 10% FBS | DMEM + 20% FBS | 4-D20-5 | NA |
| DMEM + 10% FBS | DMEM + 20% FBS | 4-D20-6 | NA |
| DMEM + 10% FBS | DMEM + 20% FBS | 4-D20-7 | NA |
| DMEM + 10% FBS | DMEM + 20% FBS | 4-D20-8 | 84 |
| DMEM + 10% FBS | DMEM + 20% FBS | 4-D20-9 | 82 |
| DMEM + 10% FBS | DMEM + 20% FBS | 4-D20-10 | NA |
| DMEM + 10% FBS | DMEM + 20% FBS | 4-D20-11 | NA |
| DMEM + 10% FBS | DMEM + 20% FBS | 4-D20-12 | NA |
| DMEM + 10% FBS | DMEM + 20% FBS | 4-D20-13 | NA |
| DMEM + 10% FBS | DMEM + 20% FBS | 4-D20-14 | 229 |
| DMEM + 10% FBS | DMEM + 20% FBS | 4-D20-15 | NA |
| DMEM + 10% FBS | DMEM + 20% FBS | 4-D20-16 | NA |
| DMEM + 10% FBS | Melanocyte | MEL-1 | NA |
| DMEM + 10% FBS | Melanocyte | MEL-2 | 268 |

### Example 41

### Initial Heterogeneity Generated by the Addition of Defined Factors

Human embryonic stem (hES) cell line H-9 was cultured as described according to the methods of the invention and then passage 45 cells were plated in a standard 6 well tissue culture plate on a feeder layer of mouse embryonic fibroblasts and allowed to overgrow for 8 days to confluence. The cells were trypsinized (0.05% trypsin) and plated into 12 wells of a Corning 12-well tissue culture plate containing mouse feeder cells and allowed to overgrow (9 days). Differentiation factors (Table I) were added to the wells with each individual factor added to 3 wells of the 12 well plate (4 factors×3 wells=12 wells total). The medium containing the differentiation factors was changed daily. The four factors were all trans retinoic acid (1 uM), recombinant human EGF (50 ng/mL), recombinant human bFGF (5 ng/mL), and recombinant human VEGFB (50 ng/mL).

About 3-6 days in the differentiating medium, the overgrown cells spontaneously detached from each well of the plate and formed a large embryoid body and a few smaller embryoid bodies. The embryoid bodies were allowed to differentiate in the presence of the factors. Each week, for 3 weeks, one well of embryoid bodies treated with each factor were harvested (4 wells per week). The embryoid bodies from each well were carefully collected, washed in phosphate buffered saline, dissociated into single cells with trypsin (0.25% trypsin) and cryopreserved for later use.

All the cryopreserved cells from above were thawed, washed and equally distributed among the 12 wells of a 12 well plate. Cells treated with each factor were aliquoted into their own plate (4 factors=4 plates). The 12 wells of each plate were filled with 1 ml of 12 different medium (Table XIX) and the cells in the 4-12 well plates were allowed to grow to confluence.

TABLE XIX
Growth Media

| | Medium | Manufacturer | Catolog Number |
|---|---|---|---|
| 1 | Airway Eiphelial Growth | PromoCell | C-21260 |
| | Medium | | |
| 2 | Epi-Life (LSGS) Medium. | Cascade | M-EPIcf/PRF-500 |
| 3 | Neurobasal Medium - B27 | Gibco | 12348-017 |
| 4 | MesenCult | Stem Cell | 5041 |
| | | Technologies | |
| 5 | HepatoZyme-SFM | Gibco | 17705-021 |
| 6 | Epi-Life (HKGS) Medium. | Cascade | M-EPIcf/PRF-500 |
| 7 | Endothelial Cell Growth | PromoCell | C-22221 |
| | Medium | | |
| 8 | Endothelial Cell SFM | Gibco | 11111-044 |
| 9 | Skeletal Muscle Cell Growth | PromoCell | C-23260 |
| | Medium | | |
| 10 | Smooth Muscle Basal Medium | PromoCell | C-22262 |
| 11 | DMEM + 20% FBS | Hyclone | SH302285 |
| 12 | Melanocyte Growth Medium | PromoCell | C-24010 |

Only a few wells had viable cells that grew to confluence and the cells from those wells were plated out at clonal densities in 15 cm cell culture dishes (250 cells/15 cm dish, 500 cells/15 cm dish and 1,000 cells/15 cm plate). The cell clones were allowed to grow undisturbed for 14 days and individual colonies picked with cloning rings and transferred to wells of a 24 well plate. Colonies that reached confluence in 24 well plates were transferred to individual wells of a 12 well plate and then to a 6 well plate on reaching confluence in the 12 well plate.

The cells of the 6 well plate were split into 3 parts for different purposes: a) T-25 cm² flasks for expanding the cell line. b) 6 cm dishes for RNA gene expression profiling and c) 8 well microscope slides for immunophenotype analysis.

On confluence, the cells in the T-25 cm² flask were transferred to a T-75 cm² flask and then to a T-150 cm². From a confluent T-150 cm² flask, the cells were transferred to a roller bottle to expand the cell line to obtain a supply for cryostorage. For cryostorage, aliquots of approximately 5 million cells were cryopreserved for later use. mRNA extraction and microarray analysis was performed. The cell lines obtained are shown below.

| Media 1. Overgrown H- | | | |
|---|---|---|---|
| 9 hES cells treated with | | | |
| differentiation factors (in | Media 2. | | |
| hES Media minus LIF | Cloning/Expansion | | |
| and bFGF)□ | Medium□□□ | Clone Name□□□□□ | ACTC Number□□□□□ |
| Retinoic Acid (10⁻⁶M) | PromoCell Endothelial | RA-PEND-1 | NA |
| Retinoic Acid (10⁻⁶M) | PromoCell Endothelial | RA-PEND-2 | NA |
| Retinoic Acid (10⁻⁶M) | PromoCell Endothelial | RA-PEND-3 | NA |
| Retinoic Acid (10⁻⁶M) | PromoCell Endothelial | RA-PEND-4 | 128 |
| Retinoic Acid (10⁻⁶M) | PromoCell Endothelial | RA-PEND-5 | NA |
| Retinoic Acid (10⁻⁶M) | PromoCell Endothelial | RA-PEND-6 | 122 |
| Retinoic Acid (10⁻⁶M) | PromoCell Endothelial | RA-PEND-7 | NA |
| Retinoic Acid (10⁻⁶M) | PromoCell Endothelial | RA-PEND-8 | NA |
| Retinoic Acid (10⁻⁶M) | PromoCell Endothelial | RA-PEND-9 | NA |
| Retinoic Acid (10⁻⁶M) | PromoCell Endothelial | RA-PEND-10 | 123 |
| Retinoic Acid (10⁻⁶M) | PromoCell Endothelial | RA-PEND-11 | NA |
| Retinoic Acid (10⁻⁶M) | PromoCell Endothelial | RA-PEND-12 | NA |
| Retinoic Acid (10⁻⁶M) | PromoCell Endothelial | RA-PEND-13 | NA |
| Retinoic Acid (10⁻⁶M) | PromoCell Endothelial | RA-PEND-14 | NA |
| Retinoic Acid (10⁻⁶M) | PromoCell Endothelial | RA-PEND-15 | 111 |
| Retinoic Acid (10⁻⁶M) | PromoCell Endothelial | RA-PEND-16 | 155 |
| Retinoic Acid (10⁻⁶M) | PromoCell Endothelial | RA-PEND-17 | NA |
| Retinoic Acid (10⁻⁶M) | PromoCell Endothelial | RA-PEND-18 | 129 |
| Retinoic Acid (10⁻⁶M) | PromoCell Endothelial | RA-PEND-19 | 130 |
| Retinoic Acid (10⁻⁶M) | PromoCell Endothelial | RA-PEND-20 | NA |
| Retinoic Acid (10⁻⁶M) | PromoCell Endothelial | RA-PEND-21 | NA |
| Retinoic Acid (10⁻⁶M) | PromoCell Endothelial | RA-PEND-22 | NA |
| Retinoic Acid (10⁻⁶M) | PromoCell Endothelial | RA-PEND-23 | NA |
| Retinoic Acid (10⁻⁶M) | PromoCell Endothelial | RA-PEND-24 | NA |
| Retinoic Acid (10⁻⁶M) | PromoCell Skeletal | RA-SKEL-1 | NA |
| Retinoic Acid (10⁻⁶M) | PromoCell Skeletal | RA-SKEL-2 | NA |
| Retinoic Acid (10⁻⁶M) | PromoCell Skeletal | RA-SKEL-3 | 124 |
| Retinoic Acid (10⁻⁶M) | PromoCell Skeletal | RA-SKEL-4 | NA |
| Retinoic Acid (10⁻⁶M) | PromoCell Skeletal | RA-SKEL-5 | NA |
| Retinoic Acid (10⁻⁶M) | PromoCell Skeletal | RA-SKEL-6 | NA |
| Retinoic Acid (10⁻⁶M) | PromoCell Skeletal | RA-SKEL-7 | NA |
| Retinoic Acid (10⁻⁶M) | PromoCell Skeletal | RA-SKEL-8 | 109 |
| Retinoic Acid (10⁻⁶M) | PromoCell Skeletal | RA-SKEL-9 | 265 |
| Retinoic Acid (10⁻⁶M) | PromoCell Skeletal | RA-SKEL-10 | NA |
| Retinoic Acid (10⁻⁶M) | PromoCell Skeletal | RA-SKEL-11 | 153 |
| Retinoic Acid (10⁻⁶M) | PromoCell Skeletal | RA-SKEL-12 | NA |
| Retinoic Acid (10⁻⁶M) | PromoCell Skeletal | RA-SKEL-13 | NA |
| Retinoic Acid (10⁻⁶M) | PromoCell Skeletal | RA-SKEL-14 | NA |
| Retinoic Acid (10⁻⁶M) | PromoCell Skeletal | RA-SKEL-15 | NA |
| Retinoic Acid (10⁻⁶M) | PromoCell Skeletal | RA-SKEL-16 | NA |
| Retinoic Acid (10⁻⁶M) | PromoCell Skeletal | RA-SKEL-17 | NA |
| Retinoic Acid (10⁻⁶M) | PromoCell Skeletal | RA-SKEL-18 | NA |
| Retinoic Acid (10⁻⁶M) | PromoCell Skeletal | RA-SKEL-19 | NA |
| Retinoic Acid (10⁻⁶M) | PromoCell Skeletal | RA-SKEL-20 | NA |
| Retinoic Acid (10⁻⁶M) | PromoCell Skeletal | RA-SKEL-21 | 125 |
| Retinoic Acid (10⁻⁶M) | PromoCell Skeletal | RA-SKEL-22 | NA |
| Retinoic Acid (10⁻⁶M) | PromoCell Skeletal | RA-SKEL-23 | NA |
| Retinoic Acid (10⁻⁶M) | PromoCell Skeletal | RA-SKEL-24 | NA |
| Retinoic Acid (10⁻⁶M) | PromoCell Smooth | RA-SMO-1 | NA |
| Retinoic Acid (10⁻⁶M) | PromoCell Smooth | RA-SMO-2 | NA |
| Retinoic Acid (10⁻⁶M) | PromoCell Smooth | RA-SMO-3 | NA |
| Retinoic Acid (10⁻⁶M) | PromoCell Smooth | RA-SMO-4 | NA |
| Retinoic Acid (10⁻⁶M) | PromoCell Smooth | RA-SMO-5 | NA |
| Retinoic Acid (10⁻⁶M) | PromoCell Smooth | RA-SMO-6 | NA |
| Retinoic Acid (10⁻⁶M) | PromoCell Smooth | RA-SMO-7 | NA |
| Retinoic Acid (10⁻⁶M) | PromoCell Smooth | RA-SMO-8 | NA |
| Retinoic Acid (10⁻⁶M) | PromoCell Smooth | RA-SMO-9 | NA |
| Retinoic Acid (10⁻⁶M) | PromoCell Smooth | RA-SMO-10 | NA |
| Retinoic Acid (10⁻⁶M) | PromoCell Smooth | RA-SMO-11 | NA |
| Retinoic Acid (10⁻⁶M) | PromoCell Smooth | RA-SMO-12 | 154 |
| Retinoic Acid (10⁻⁶M) | PromoCell Smooth | RA-SMO-13 | NA |
| Retinoic Acid (10⁻⁶M) | PromoCell Smooth | RA-SMO-14 | NA |
| Retinoic Acid (10⁻⁶M) | PromoCell Smooth | RA-SMO-15 | NA |
| Retinoic Acid (10⁻⁶M) | PromoCell Smooth | RA-SMO-16 | NA |
| Retinoic Acid (10⁻⁶M) | PromoCell Smooth | RA-SMO-17 | NA |
| Retinoic Acid (10⁻⁶M) | PromoCell Smooth | RA-SMO-18 | NA |
| Retinoic Acid (10⁻⁶M) | PromoCell Smooth | RA-SMO-19 | 232 |
| Retinoic Acid (10⁻⁶M) | PromoCell Smooth | RA-SMO-20 | NA |
| Retinoic Acid (10⁻⁶M) | PromoCell Smooth | RA-SMO-21 | NA |
| Retinoic Acid (10⁻⁶M) | PromoCell Smooth | RA-SMO-22 | NA |
| Retinoic Acid (10⁻⁶M) | PromoCell Smooth | RA-SMO-23 | NA |
| Retinoic Acid (10⁻⁶M) | PromoCell Smooth | RA-SMO-24 | NA |
| Retinoic Acid (10⁻⁶M) | DMEM + 20% FBS | RA-D20-1 | NA |
| Retinoic Acid (10⁻⁶M) | DMEM + 20% FBS | RA-D20-2 | NA |
| Retinoic Acid (10⁻⁶M) | DMEM + 20% FBS | RA-D20-3 | NA |
| Retinoic Acid (10⁻⁶M) | DMEM + 20% FBS | RA-D20-4 | NA |
| Retinoic Acid (10⁻⁶M) | DMEM + 20% FBS | RA-D20-5 | 226 |
| Retinoic Acid (10⁻⁶M) | DMEM + 20% FBS | RA-D20-6 | 212 |
| Retinoic Acid (10⁻⁶M) | DMEM + 20% FBS | RA-D20-7 | NA |
| Retinoic Acid (10⁻⁶M) | DMEM + 20% FBS | RA-D20-8 | NA |
| Retinoic Acid (10⁻⁶M) | DMEM + 20% FBS | RA-D20-9 | NA |
| Retinoic Acid (10⁻⁶M) | DMEM + 20% FBS | RA-D20-10 | NA |
| Retinoic Acid (10⁻⁶M) | DMEM + 20% FBS | RA-D20-11 | NA |
| Retinoic Acid (10⁻⁶M) | DMEM + 20% FBS | RA-D20-12 | NA |
| Retinoic Acid (10⁻⁶M) | DMEM + 20% FBS | RA-D20-13 | NA |
| Retinoic Acid (10⁻⁶M) | DMEM + 20% FBS | RA-D20-14 | NA |
| Retinoic Acid (10⁻⁶M) | DMEM + 20% FBS | RA-D20-15 | NA |
| Retinoic Acid (10⁻⁶M) | DMEM + 20% FBS | RA-D20-16 | 155 |
| Retinoic Acid (10⁻⁶M) | DMEM + 20% FBS | RA-D20-17 | NA |
| Retinoic Acid (10⁻⁶M) | DMEM + 20% FBS | RA-D20-18 | NA |
| Retinoic Acid (10⁻⁶M) | DMEM + 20% FBS | RA-D20-19 | 230 |
| Retinoic Acid (10⁻⁶M) | DMEM + 20% FBS | RA-D20-20 | NA |
| Retinoic Acid (10⁻⁶M) | DMEM + 20% FBS | RA-D20-21 | NA |
| Retinoic Acid (10⁻⁶M) | DMEM + 20% FBS | RA-D20-22 | NA |
| Retinoic Acid (10⁻⁶M) | DMEM + 20% FBS | RA-D20-23 | NA |
| Retinoic Acid (10⁻⁶M) | DMEM + 20% FBS | RA-D20-24 | NA |
| EGF (50 ug/ml) | PromoCell Smooth | E-SMO-1 | NA |
| EGF (50 ug/ml) | PromoCell Smooth | E-SMO-2 | NA |
| EGF (50 ug/ml) | PromoCell Smooth | E-SMO-3 | NA |
| EGF (50 ug/ml) | PromoCell Smooth | E-SMO-4 | NA |
| EGF (50 ug/ml) | PromoCell Smooth | E-SMO-5 | NA |
| EGF (50 ug/ml) | PromoCell Smooth | E-SMO-6 | NA |
| EGF (50 ug/ml) | PromoCell Smooth | E-SMO-7 | NA |
| EGF (50 ug/ml) | PromoCell Smooth | E-SMO-8 | NA |
| EGF (50 ug/ml) | PromoCell Smooth | E-SMO-9 | NA |
| EGF (50 ug/ml) | PromoCell Smooth | E-SMO-10 | NA |
| EGF (50 ug/ml) | PromoCell Smooth | E-SMO-11 | NA |
| EGF (50 ug/ml) | PromoCell Smooth | E-SMO-12 | NA |
| EGF (50 ug/ml) | PromoCell Smooth | E-SMO-13 | NA |
| EGF (50 ug/ml) | PromoCell Smooth | E-SMO-14 | NA |
| EGF (50 ug/ml) | PromoCell Smooth | E-SMO-15 | NA |
| EGF (50 ug/ml) | PromoCell Smooth | E-SMO-16 | NA |
| EGF (50 ug/ml) | PromoCell Smooth | E-SMO-17 | NA |
| EGF (50 ug/ml) | PromoCell Smooth | E-SMO-18 | NA |
| EGF (50 ug/ml) | PromoCell Smooth | E-SMO-19 | NA |
| EGF (50 ug/ml) | PromoCell Smooth | E-SMO-20 | NA |
| EGF (50 ug/ml) | PromoCell Smooth | E-SMO-21 | NA |
| EGF (50 ug/ml) | PromoCell Smooth | E-SMO-22 | NA |
| EGF (50 ug/ml) | PromoCell Smooth | E-SMO-23 | NA |
| EGF (50 ug/ml) | PromoCell Smooth | E-SMO-24 | NA |
| Basic FGF (5 ng/ml) | PromoCell Endothelial | F-PEND-1 | NA |
| Basic FGF (5 ng/ml) | PromoCell Endothelial | F-PEND-2 | NA |
| Basic FGF (5 ng/ml) | PromoCell Endothelial | F-PEND-3 | NA |
| Basic FGF (5 ng/ml) | PromoCell Endothelial | F-PEND-4 | NA |
| Basic FGF (5 ng/ml) | PromoCell Endothelial | F-PEND-5 | NA |
| Basic FGF (5 ng/ml) | PromoCell Endothelial | F-PEND-6 | NA |
| Basic FGF (5 ng/ml) | PromoCell Endothelial | F-PEND-7 | NA |
| Basic FGF (5 ng/ml) | PromoCell Endothelial | F-PEND-8 | NA |
| Basic FGF (5 ng/ml) | PromoCell Endothelial | F-PEND-9 | NA |
| Basic FGF (5 ng/ml) | PromoCell Endothelial | F-PEND-10 | NA |
| Basic FGF (5 ng/ml) | PromoCell Endothelial | F-PEND-11 | NA |
| Basic FGF (5 ng/ml) | PromoCell Endothelial | F-PEND-12 | NA |
| Basic FGF (5 ng/ml) | PromoCell Endothelial | F-PEND-13 | NA |
| Basic FGF (5 ng/ml) | PromoCell Endothelial | F-PEND-14 | NA |
| Basic FGF (5 ng/ml) | PromoCell Endothelial | F-PEND-15 | NA |
| Basic FGF (5 ng/ml) | PromoCell Endothelial | F-PEND-16 | NA |
| Basic FGF (5 ng/ml) | PromoCell Endothelial | F-PEND-17 | NA |
| Basic FGF (5 ng/ml) | PromoCell Endothelial | F-PEND-18 | NA |
| Basic FGF (5 ng/ml) | PromoCell Endothelial | F-PEND-19 | NA |
| Basic FGF (5 ng/ml) | PromoCell Endothelial | F-PEND-20 | NA |
| Basic FGF (5 ng/ml) | PromoCell Endothelial | F-PEND-21 | NA |
| Basic FGF (5 ng/ml) | PromoCell Endothelial | F-PEND-22 | NA |
| Basic FGF (5 ng/ml) | PromoCell Endothelial | F-PEND-23 | NA |
| Basic FGF (5 ng/ml) | PromoCell Endothelial | F-PEND-24 | NA |
| VEGF (50 ng/ml) | PromoCell Endothelial | V-PEND-1 | NA |
| VEGF (50 ng/ml) | PromoCell Endothelial | V-PEND-2 | NA |
| VEGF (50 ng/ml) | PromoCell Endothelial | V-PEND-3 | NA |
| VEGF (50 ng/ml) | PromoCell Endothelial | V-PEND-4 | NA |
| VEGF (50 ng/ml) | PromoCell Endothelial | V-PEND-5 | NA |
| VEGF (50 ng/ml) | PromoCell Endothelial | V-PEND-6 | NA |
| VEGF (50 ng/ml) | PromoCell Endothelial | V-PEND-7 | NA |
| VEGF (50 ng/ml) | PromoCell Endothelial | V-PEND-8 | NA |
| VEGF (50 ng/ml) | PromoCell Endothelial | V-PEND-9 | NA |
| VEGF (50 ng/ml) | PromoCell Endothelial | V-PEND-10 | NA |
| VEGF (50 ng/ml) | PromoCell Endothelial | V-PEND-11 | NA |
| VEGF (50 ng/ml) | PromoCell Endothelial | V-PEND-12 | NA |
| VEGF (50 ng/ml) | PromoCell Endothelial | V-PEND-13 | NA |
| VEGF (50 ng/ml) | PromoCell Endothelial | V-PEND-14 | NA |
| VEGF (50 ng/ml) | PromoCell Endothelial | V-PEND-15 | NA |
| VEGF (50 ng/ml) | PromoCell Endothelial | V-PEND-16 | NA |
| VEGF (50 ng/ml) | PromoCell Endothelial | V-PEND-17 | NA |
| VEGF (50 ng/ml) | PromoCell Endothelial | V-PEND-18 | NA |
| VEGF (50 ng/ml) | PromoCell Endothelial | V-PEND-19 | NA |
| VEGF (50 ng/ml) | PromoCell Endothelial | V-PEND-20 | NA |
| VEGF (50 ng/ml) | PromoCell Endothelial | V-PEND-21 | NA |
| VEGF (50 ng/ml) | PromoCell Endothelial | V-PEND-22 | NA |
| VEGF (50 ng/ml) | PromoCell Endothelial | V-PEND-23 | NA |
| VEGF (50 ng/ml) | PromoCell Endothelial | V-PEND-24 | NA |

### Example 42

### Laser Capture Microscopy and Microarray Analysis of Whole Organism Tissues, hES, and Differentiated hES Cell Lines

The quantitation of gene expression in whole organism tissues, human embryonic stem cells, and their differentiated progeny, are accomplished by microarray technologies well know to those versed in the art. Tissue samples from biopsies and cell colonies containing differentiated hES cell progeny may be isolated using Laser Capture Microdissection (LCM) to capture small populations of cell for analysis (Baba, et al, 2006, Trans. Res. 148:103-113, Sluka, P. et al, 2002, Biol Repro 67:820-828). In this approach, total RNA is purified from target cells, cell colonies, or tissues and RNA prepared by linear amplification with T7 RNA polymerase such that there is a linear appearance of mRNA product in direct proportion to the amount of RNA template in the samples. These amplified samples are then fluorescently labeled and gene expression levels determined using microarray analysis.

### Selective Collection of Cells by LCM

Biopsy specimens are embedded in Tissue-Tek O.C.T. Compound (Miles, Inc., Elkhart, hid) and frozen in acetone chilled with dry ice. Ten micrometer frozen sections are produced, fixed in a 70% ethanol solution, and stained with hematoxylin and eosin. Cell clusters are selectively picked up by LCM (LM-100; Arcturus Engineering, Inc., Mountain View, Calif.) following the standard protocol as previously described (Emmert-Buck M R, Bonner R F, Smith P D, Chuaqui R F, Zhuang Z, Goldstein S R et al. Laser capture micro-dissection. Science (Wash. DC) 1996; 274:998-1001, Bonner R F, Emmert-Buck M R, Cole K, Pohida T, Chuaqui R, Goldstein S, et al. Laser capture dissection: molecular analysis of tissue. Science (Wash. DC) 1997; 278:1481-2). The entire sampling scheme is repeated three times from the same tissue. LCM is performed using a PixCell II laser capture microdissection microscope (Arcturus Engineering, Mountain View, Calif.), equipped with a fluorescence light source. Each section is pretreated with a PrepStrip tissue preparation strip (Arcturus) to remove loose debris and to flatten the tissue. Sections are then visualized using a 20× objective, and capture is performed using a 30-mm diameter laser spot size set at 20-30 mW with a pulse duration of 5 msec. Cells are captured using CapSure LCM caps (Arcturus) and stored in a desiccator prior to extraction of total RNA.

Extraction of Total RNA from BEC

Total RNA is isolated from the collected cells using a StrataPrep Total RNA Microprep Kit (Stratagene, La Jolla, Calif.), according to the manufacturer's instructions. A preliminary examination is conducted to confirm the quality of the tissues as follows: Total RNA was extracted from the remaining portion of specimens using TRIzol (Gibco BRl, Rockville, Md.) and analyzed by electrophoresis in formaldehyde-agarose gels.

### Gene Amplification by T7 RNA Polymerase

Total RNA extracted from the collected cells is linearly amplified using T7 RNA polymerase, with a MessageAmp aRNA Kit (Ambion, Austin, Tex.). The applied procedure consists of reverse transcription with an oligo (dT) primer bearing a T7 promoter, and in vitro transcription of the resulting DNA with T7 RNA polymerase, generating hundreds to thousands of antisense cRNA copies of each mRNA per sample. To confirm the efficiency and accuracy of the gene amplification procedure, a preliminary examination is performed using a sample of human ovary total RNA (Stratagene, La Jolla, Calif.) as follows. First, 2 μg of human ovary total RNA is amplified twice by the gene amplification procedure. The resulting amount of amplified RNA is then determined and compared with that of the original. Secondly, the genetic composition of the amplified RNA is compared with that of the original by cRNA microarray analysis. cRNA probes are labeled with fluorescent dye, generated using an Illumina Total Prep RNA Labelling kit (Ambion, Inc, Austin, Tex.), from samples of (1) original human ovary total RNA, (2) RNA after refining poly(A)_mRNA (OligotexdT30, (Super)mRNA Purification Kit; Takara Bio, Inc.), (3) RNA after single amplification, and (4) RNA after amplifying twice. All samples are hybridized on a cRNA microarray (Illumina Human Sentrix 6 Beadchip, Illumina, Inc, San Diego, Calif.), and the fluorescence signals of the resulting spots are scanned by an Illumina 500 Beadstation. Correlations are examined by constructing scatter plots of the logarithms of the resulting fluorescent signals. The expression of each gene can be simultaneously analyzed through hybridization of the probes, which are prepared by using RNA obtained from human cells as a template. Control spots can be used to normalize the signal intensity between fluorescence-labeled probes and to determine the background level.

cRNA Microarray Analysis

cRNA probes are generated from the LCM generated RNA samples, amplified twice and labeled with fluorescent dye (Illumina Total Prep RNA Labelling kit, Ambion, Inc, Austin, Tex.). The labelled cRNA probes are then hybridized on an Illumina Human Sentrix-6 microarray and scanned as described above.

### Example 43

### Generation of Canines Secreting the TAT-Tag Fusion Protein

### Construction of TAT-TAg Expression Plasmid

The SV40 large T antigen is amplified by polymerase chain reaction (PCR) with primers flanking the open reading frame. The 5′ PCR oligonucleotide sequence included DNA sequence complementary to the 5′ end of the SV40 large T antigen and DNA sequence encoding the TAT PTD (YGRKKRRQRRR). The PCR product was cloned into the pEF6/V5-His TOPO® TA vector (Invitrogen, Carlsbad, Calif.) according to the manufacturer instructions. Transcription is under the control of the hEF-1alpha promoter (hEF-1alpha) and the fusion protein (TAT-TAg) contains at its C-terminal end a myc and his epitope tags.

### Cell Culture, Transfection, and Replication Labeling

Human cell lines are grown as described above, by the supplying vendor or collaborator, or in DMEM supplemented with 10% fetal bovine serum, 1× glutamax, and nonessential amino acids. To create cell lines secreting TAT-Tag, the human Hela cell line is transfected with the TAT-large T antigen construct using GenePorter Transfection Reagent (Gene Therapy Systems, San Diego, Calif.) by mixing 7 μg of plasmid DNA in 1 ml serum-free DMEM and mixing with 1 ml DMEM containing 35 μl GenePorter reagent. After aspirating medium from a 60 mm culture dish with Hela cells, this solution is added to the cells. After 5 hrs, 2 ml of DMEM containing 20% FCS is added. After another 48 hrs, the drug blasticidin is added to the cultures to select for stable Hela cell transfectants. Blasticidin resistant colonies are picked, expanded and the cell conditioned medium analyzed for the presence of the TAT-Tag fusion protein by immunoblotting cell extracts, conditioned medium and cell pellet as described below.

### Antibodies

The following primary antibodies are used: anti-myc tag mouse monoclonal antibody (clone 9E10); anti-his tag mouse monoclonal antibody (Dianova, Hamburg, Germany); anti-SV40 large T antigen mouse monoclonal antibody (PAB 101). For immunoblot analysis, horseradish peroxidase-conjugated anti-mouse IgG (Amersham, Buckinghamshire, U.K.) is used.

### Immunoblot Analysis

Transfected COS-7 cells are extracted for 30 min on ice in RIPA buffer. In brief, we analyze cell extracts and cell pellets by immunoblot using anti-myc tag mouse monoclonal antibody to detect the TAT-Tag fusion protein.

### Cell Co-Culture

TAT-Tag secreting Hela cell lines are used to treat growth medium appropriate for culture of the recipient cell lines. Briefly, TAT-Tag secreting Hela cells are cultured in growth medium. The medium is harvested by aspiration, filtered and applied to recipient cell cultures. Uptake of the TAT-Tag by recipient cells is monitored by immunoblotting as described above.

### Example 44

### Mitomycin C Treatment of Cells

1. Grow cells to confluence in 15 cm plates or T-150 flasks. 2. Inject 2 ml of sterile water (or PBS) into Mitomycin C (Sigma, Cat# M4287-2MG) vial and dissolve completely. Concentration of Mitomycin C is 1 mg/ml. Once prepared, Mitomycin C is good for about 2 weeks when stored at 4 degree C. 3.

Prepare about 10 ml of warm medium for each plate or flask. Add 100 ul of Mitomycin C to each 10 ml of medium. Concentration of Mitomycin C is 10 ug/ml. 4. Aspirate medium from the plates or flasks and replace with the Mitomycin C medium (10 ml per plate or flask). Place in CO2 incubator at 37 degree C. for 3 hours. 5. Aspirate Mitomycin C medium into disposal trap that containing bleach. Wash Mitomycin C treated cells 2-4 times with warm PBS. Aspirate PBS into bleach containing trap. 6. Trypsinize cells, neutralize the Trypsin with DMEM+10% FBS and count the number of cells with a Coulter Counter or hemacytometer. 7. Determine the number of cells needed to cover the vessel of interest. For example, for mouse embryonic fibroblasts (MEF) feeder cells, at least 500K cells for one well of a 6 well plate are needed. This cell number could be increased by approximately 10-30% to account for cell death during the freezing process. 8. Freeze the cells in aliquots convenient for later use. For example, MEF feeder cells can be frozen in aliquots for single wells (650K), 3 wells (1.75 million) or 6 wells (3.3 million). Freezing medium is the same medium used to grow the cells containing 10% dimethylsulfoxide (DMSO) and freezing solution should be cooled to 2-4 degree C. prior to use. Do not use DMSO freezing medium warmed to 37 degree C. Medium should contain at least 10% serum for best results. 9. Before discarding any unused Mitomycin C or vessels used in the inactivation procedure, treat with bleach.

### Example 45

The cells of this invention (made by the methods of this invention) are useful in the delivery of members of the EGF family of growth factors to tissue for therapeutic effect or for the delivery of such factors to other cells to generate the initial heterogeneous mixture of cells of this invention or for the enrichment or clonal or oligoclonal propagation steps of the methods of this invention. By way of nonlimiting example, the EGF family member AREG (accession number NM_{—}001657.2) is expressed at relatively high levels by the following cell lines produced by the methods of this invention: Cell line 4, SM8, EN7, EN13 (ACTC174), SK5, and EN47 (ACTC176). The methods of derivation and propagation of these cells are described herein. Since these cells express relatively high levels of AREG, they are useful for therapeutic use in the treatment of disorders wherein therapeutic effect is imparted by inducing the proliferation of epithelial cells including the treatment of burns and nonhealing ulcers through the stimulation of keratinocyte proliferation, the induction of the proliferation of the parenchymal cells of the liver such as after liver injury, surgical resection of the liver after the removal of a portion of the liver due to cancer or the induction of the growth of the liver in cirrhosis, the activation of osteoblasts to increase the production of new bone. They are also useful in inducing the initial heterogeneous mixture of cells of the methods of this invention in that they induce or increase the percentage of cells in the heterogeneous mixture of osteoblastic, smooth muscle, and epithelial lineages including keratinocytes, respiratory, middle ear mucosa, intestinal, conjunctival, oral mucosal, mammary, prostatic, pancreatic duct, and urinary tract epithelium. Lastly, these cells expressing relatively high levels of AREG are useful in inducing the proliferation of these same cells in the enrichment step or the clonal propagation step by the use of medium conditioned by these cells or by the co-culture of the cells, or the use of the cells secreting this factor as feeder cells as described herein.

### Example 46

The cells of this invention (made by the methods of this invention) are useful in the delivery of members of the TGFbeta family of growth factors to tissue for therapeutic effect or for the delivery of such factors to other cells to generate the initial heterogeneous mixture of cells of the present invention or for the enrichment or clonal or oligoclonal propagation steps of the present invention. By way of nonlimiting example, the TGFbeta family member BMP4 (accession number NM_{—}130851.1) is expressed at relatively high levels by the following cell lines produced by the methods of this invention: Cell line ELS5-6 (ACTC118), J8, B10, 4-3, B16 (ACTC59), E75 (ACTC102), E72 (ACTC100), 2-2 (ACTC62), B28 (ACTC60), B7 (ACTC53), 6-1 (ACTC64), B2 (ACTC51), 2-1 (ACTC63), B11 (ACTC58), 2-3 (ACTC70), CM10-4, CM30-5, CM0-5, 4, B22, 6, CM30-2 (ACTC78), B15 (ACTC71), B20, B27, 2, 4-4, B9, CM10-1, 5-4 (ACTC68), and B17 (ACTC54). Another nonlimiting example of a TGFbeta family member unexpectedly produced at relatively high levels in the cell lines produced by the methods of this invention includes BMP6 (accession number NM_{—}001718.2). It is expressed at relatively high levels by the following cell lines produced by the methods of this invention: B16 (ACTC59), E75 (ACTC102), 2-2 (ACTC62), B7 (ACTC53), (ACTC64), B2 (ACTC51), 2-1 (ACTC63), B11 (ACTC58), 2-3 (ACTC70), CM20-4 (ACTC79), CM10-4, CM30-5, CM50-5 (ACTC75), E51 (ACTC86), and B17 (ACTC54). The methods of derivation and propagation of these cells are described herein. Since these cells express relatively high levels of BMP4 and/or BMP6 and members of the TGFbeta family are potent inducers of endochondral osteogenesis, they are useful for therapeutic use in the activation of osteoblasts to increase the production of new bone, such as to improve the rate of the healing of bone fractures and to increase the bone mass in the treatment of osteoporosis. Numerous strategies to deliver BMP4 or BMP6 to the site of bone loss have been described, such as the direct injection of the factor, slow release devices, viral gene therapy, and the transfection of the gene into a cell type that can be transplanted into the site of injury. The cells of this invention are unique and an improvement over previous techniques for delivering BMP4 or BMP6, in that the cells described in this example that express relatively high levels of BMP4 or BMP6 are normal human cells in the process of embryonic development, and the high levels of expression of BMP4 or BMP6 can be modified in vivo either to increase or decrease the expression of the gene as needed physiologically. They are also useful in inducing the initial heterogeneous mixture of cells of the present invention in that they induce or increase the percentage of cells in the heterogeneous mixture of osteoblastic, and epithelial lineages including keratinocytes, respiratory, intestinal, oral mucosal, mammary, prostate, and urinary tract epithelium. Lastly, these cells expressing relatively high levels of BMP4 and BMP6 are useful in inducing the proliferation of these osteoblast cells in the enrichment step or the clonal propagation step by the use of medium conditioned by these cells or by the co-culture of the cells, or the use of the cells secreting this factor as feeder cells as described herein.

Another nonlimiting example are those cell lines of their invention that unexpectedly express relatively high levels of the TGFbeta family member TGFbeta3 and useful for therapeutic effect or for the delivery of such factors to other cells to generate the initial heterogeneous mixture of cells of the present invention or for the enrichment or clonal or oligoclonal propagation steps of the present invention. TGFbeta3 (accession number NM_{—}003239.1) is expressed at relatively high levels by the following cell lines produced by the present invention: C4ELSR_{—}1, C4ELSR_{—}2, E45 (ACTC99), E51 (ACTC86), E33 (ACTC114), EN7, and EN13 (ACTC174). The methods of derivation and propagation of these cells are described herein. Since these cells express relatively high levels of TGFbeta3, they are useful for therapeutic use in the treatment of nonhealing skin ulcers, such as to improve the rate of the healing of the skin in the treatment of burns, decubitus and stasis ulcers, and diabetic ulcers. The cells of the present invention are unique and an improvement over previous techniques for delivering TGFbeta3, in that the cells described in this example that express relatively high levels of the factor, are normal human cells in the process of embryonic development, and the high levels of expression of the factor can be modified in vivo either to increase or decrease the expression of the gene as needed physiologically. In addition, the cells can be mitotically inactivated and assembled onto a matrix such that the cells function in a device to locally produce the factor for a limited period of time. They are also useful in inducing the initial heterogeneous mixture of cells of the present invention in that they induce or increase the percentage of cells in the heterogeneous mixture of muscle satellite, mesenchymal, and endothelial cells. Lastly, these cells expressing relatively high levels of TGFbeta3 are useful in inducing the proliferation of muscle satellite, mesenchymal, and endothelial cells in the enrichment step or the clonal propagation step by the use of medium conditioned by these cells or by the co-culture of the cells, or the use of the cells secreting this factor as feeder cells as described herein.

### Example 47

A subset of the cells of this invention have the unexpected property of a relatively high level of expression of follistatin (FST, accession number NM_{—}013409.1). These cells have use in the delivery of FST to tissue for therapeutic effect or for the delivery of such factors to other cells to generate the initial heterogeneous mixture of cells of the present invention or for the enrichment or clonal or oligoclonal propagation steps of the present invention. By way of nonlimiting example, FST is expressed at relatively high levels by the following cell lines produced by this invention: C4ELSR_{—}1, C4ELSR_{—}2, SM8. SM25 (ACTC166), Z8 (ACTC213), SM17 (ACTC182), SM33 (ACTC183), SM4 (ACTC143), SM42 (ACTC149), Z7 (ACTC200), SM2 (ACTC142), SK50 (ACTC159), SM49 (ACTC151), EN2 (ACTC139), SM22 (ACTC156), and EN47 (ACTC176). The methods of derivation and propagation of these cells are described herein. Since these cells express relatively high levels of FST, they are useful for therapeutic use in the treatment of disorders wherein therapeutic effect is imparted by inhibiting the activity of TGFbeta pathways including the treatment of rare disorders such as fibrodysplasia ossificans progressiva characterized by heterotopic ossification of para-vertebral musculature. The introduction of the cells of the present invention are therefore useful in antagonizing these pathways and in reducing such heterotopic bone formation. In addition, the inhibition of the activity of the TGFbeta family member Activin A in arteriosclerosis using the cells of the present invention is useful in inhibiting smooth muscle proliferation and thereby reducing the risk of myocardial infarction. Similarly, these FST-expressing cells are useful in antagonizing the inhibitory activity of Activin A on muscle growth and repair such that these cells expressing relatively high levels of FST if implanted into regions of skeletal muscle in need of growth and repair result in increased muscle mass. The cells of this example expressing relatively high levels of FST are also useful in inducing the initial heterogeneous mixture of cells of the present invention in that they induce or increase the percentage of cells in the heterogeneous mixture of cytotrophoblasts and muscle stem cells. Lastly, these cells expressing relatively high levels of FST are useful in inducing the proliferation of these same cells in the enrichment step or the clonal propagation step by the use of medium conditioned by these cells or by the co-culture of the cells, or the use of the cells secreting this factor as feeder cells as described herein.

### Example 48

Human embryos are attached to collagen-coated tissue culture vessels and cells from the ICM are allowed to attach and spread in SR medium containing 1% DMSO. The cultures are fed daily with SR medium for 4 days and then exchanged into unconditioned SR medium containing both 1% DMSO and 2.5% Na-butyrate, with which they are fed daily for 6 days. They are then replated onto collagen, and cultured in a hepatocyte maturation medium containing: 30 ng/mL hEGF+1% DMSO 1% DMSO+10 ng/mL TGF-\acute{α}+2.5 mM 30 ng/mL HGF+butyrate 2.5 mM butyrate (see U.S. Pat. No. 7,033,831).

The differentiated cells are allowed to grow for 7-10 days to form colonies, the colonies are cloned and plated in 24-well gelatin-coated plates containing the same medium in which they are grown. The individual colonies are expanded to obtain a stock of cells and the cell line stocks are cryopreserved.

During the clonal expansion protocol of step 2, samples of the cell lines are taken for gene expression and immunophenotype analysis.

### Example 49

### Differentiation of Directly-Differentiated Embryo-Derived Cells into Neuronal Cells

Human ICMs are isolated from blastocyst-staged embryos by immunosurgery as is well-known in the art, the ICMs are cultured on tissue culture plastic for five days in Gibco Neural Basal Medium, then placed in DMEM supplemented with 10% (by volume) fetal bovine serum (FBS). After resuspension in DMEM and 10% FBS, 1×106 cells are plated in 5 ml DMEM plus 10% PBS plus 0.5 μM retinoic acid in a 60 mm Fisher brand bacteriological grade Petri dish. In such Petri dishes, embryonic stem cells cannot adhere to the dish, and instead adhere to each other, thus forming small aggregates of cells. Aggregation of cells aids in enabling proper cell differentiation. After two days, aggregates of cells are collected and resuspended in fresh DMEM plus 10% FBS plus 0.5 μM retinoic acid, and replated in Petri dishes for an additional two days. Aggregates, now induced four days with retinoic acid, are trypsinized to form a single-cell suspension, and plated in medium on poly-D-lysine-coated tissue culture grade dishes. The stem cell medium is formulated with Kaighn's modified Ham's F12 as the basal medium with the following supplements added: 15 μg/ml ascorbic acid 0.25% (by volume) calf serum 6.25 μg/ml insulin 6.25 μg/ml transferrin 6.25 μg/ml selenous acid 5.35 μg/ml linoleic acid 30 pg/ml thyroxine (T3) 3.7 ng/ml hydrocortisone 1. ng/ml Heparin 10 ng/ml somatostatin 10 ng/ml Gly-His-Lys (liver cell growth factor) 0.1 μg/ml epidermal growth factor (EGF) 50 μg/ml bovine pituitary extract (BPE) (see U.S. Pat. No. 6,432,711).

The differentiated cells are allowed to grow for 7-10 days to form colonies, the colonies are cloned and plated in 24-well gelatin-coated plates containing the same medium in which they are grown. The individual colonies are expanded to obtain a stock of cells and the cell line stocks are cryopreserved.

During the clonal expansion protocol, samples of the cell lines are taken for gene expression and immunophenotype analysis.

### Example 50

This Example is based on West et al., 2008, Regenerative Medicine vol. 3(3) pp. 287-308, which is incorporated by reference herein in its entirety, including Supplementary Tables Ito VIII. This reference and all Supplementary Data are available as of the filing date of this application at the following website: http://www.futuremedicine.com/doi/full/10.2217/17460751.3.3.287.

Human blastomeres are removed from 8 cell embryos and plated onto collagen-coated tissue culture vessels and cultured for two days in DMEM medium with 10% PBS. The cells are then removed by scraping and placed in Neural basal medium on bacteriological plates. Media is supplemented with the following growth factors: retinoic acid (Sigma): 10-7M (Bain et al (1995) or 10-6M (Bain et al., 1996); TGFâl (Sigma): 2 ng/ml (Slager et al., (1993) Dev. Genet., Vol. 14, pp. 212 224.); and âNGF (New Biotechnology, Israel): 100 ng/ml (Wobus et al., 1988). After 21 days, EBs are plated on 5 μg/cm2 collagen treated plates, either as whole EB's, or as single cells dissociated with trypsin/EDTA. The cultures are maintained for an additional week or 2 days respectively (see U.S. Pat. No. 7,045,353).

The differentiated cells are allowed to grow for 7-10 days to form colonies, the colonies are cloned according to the steps 2 (a) and 2 (b) of the present invention and plated in 24-well gelatin-coated plates containing the same medium in which they are grown. The individual colonies are expanded to obtain a stock of cells and the cell line stocks are cryopreserved.

During the clonal expansion protocol, samples of the cell lines are taken for gene expression and immunophenotype analysis.

### Example 51

Human embryonic stem (hES) cells have significant promise for medical research and cell-based therapy due to their pluripotency1,2 and presumed ability to cascade through the entire catalog of human embryonic progenitor (hEP) cell types. Embryonic progenitors are cells capable of proliferation and differentiation into one or more terminally differentiated cell types while typically expressing transcripts unique to embryonic stages of development. Embryonic progenitors are therefore usually present only during the embryonic stages of development. Examples of hEP cells include: migrating neural crest3, early ectodermal progenitors of the cerebellum4, endodermal progenitors such as those of the primordial liver5, and mesodermal precursors of hematopoietic lineages6. The isolation and culture of hEP cell lines, though largely unexplored, would facilitate the molecular characterization of these cell types and allow more precise studies of the cellular interactions that occur during the development of human tissues. Thus, there is a need for a general method of isolating hEP cell lines to a level of purity useful in basic research and for the manufacturing of such cells for therapeutic application.

The differentiation of hES cells in-vitro is not well understood and current directed differentiation protocols rely heavily on factors previously identified to be necessary for specific aspects of mouse embryonic development in vivo. Accordingly, current protocols employ a strategy wherein hES cells are expanded, exposed to specific differentiation conditions, after which the desired differentiated cell types are purified utilizing affinity-based methods. Since few such purification strategies have been perfected, current differentiation protocols are very inefficient, resulting in heterogeneous populations of differentiated cells wherein the desired cell type represents only a few percent of the population7. There are two major concerns with this strategy from a practical standpoint. First, therapeutic applications require a sufficiently pure formation to insure safety (i.e., minimal risk of contaminating cells proliferating to cause tumors or migrating and adversely affecting normal tissue function)8. Second, therapeutic applications require a robust and economical scale-up protocol. hES cells are among the most difficult of cells to propagate en masse 9 without losing pluripotency or normal karyotype. Therefore, there is a need to improved methods to increase purity and scalability of hEP cell types.

Early efforts in cell purification in vitro included attempts at purifying cells by clonal isolation. While frequently employed in purifying immortalized cells or cells well acclimated to in vitro culture such as fetal fibroblasts10, clonal isolation of most normal human cell types often fails either because suitable culture conditions cannot be identified or because the reduced telomere lengths of most fetal, neonatal, and adult cell types results in replicative senescence before a clonal line can be obtained. While mouse cells generally possess longer telomeres and labile telomerase expression, few tissues even from relatively early in embryonic development, such as E11.5-E13 mouse embryos are capable of generating stable cell lines and <1% of those can be clonally expanded (unpublished results). We reasoned, however, that hES derived hEPs might not have the same limitations as a result of their long initial telomere length and the potential to capture cells at stages of differentiation even earlier than that corresponding to E11.5 mouse cells. In addition, since homologous cells display a surprising degree of spatial diversity due to site specific homeobox expression 11 that plays an important role in embryonic pattern formation12, clonal isolates have the potential to lead to lines with a more uniform pattern of differentiated gene expression. Here we demonstrate the successful derivation of a library of human embryonic progenitor (hEP) cell lines using a novel two-step isolation method that selects clonal cell populations from hES cells grown and differentiated under a large variety of culture conditions. Many of the hEP lines may represent intermediates of human embryonic differentiation that have not previously been identified or characterized. The establishment of a library of clonal hEP cell lines as described here provides a novel and scalable source of cells for regenerative therapies and provides the first initial characterization of cell types that proliferate relatively well and are, therefore likely present in many cultures of ES-derived cells.

### Results

Multiplex Generation and Characterization of hEP Cell Clones

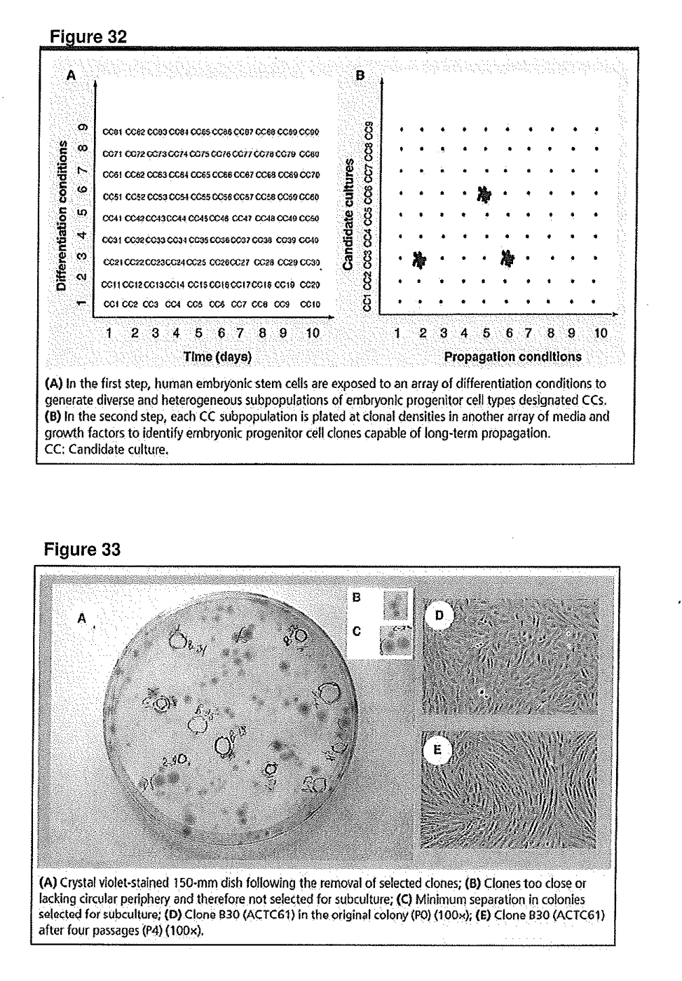

In a “shotgun” strategy to search for hEP cell types capable of propagation in vitro, we implemented a two step multiplex cell line isolation protocol designated ACTCellerate to identify differentiated hES-derived cell types capable of clonal propagation in an array of differentiation and propagation conditions (in addition to the description above for the ACTCellarate process, see U.S. Patent Publication 2008/0070303, incorporated by reference herein in its entirety). In the first step, hES cells (WA09 [H9] and MA03) were differentiated under an array of in vitro conditions that included colony in situ differentiation, differentiation as embryoid bodies (EBs), on nonadherent plastic or hanging drops, differentiation in the presence of different growth factors, and for various periods of time (specific differentiation conditions are described in methods and the conditions for each cell line are shown in Supplementary Table 1 from West et al., 2008, Regenerative Medicine vol. 3(3) pp. 287-308, which is incorporated by reference herein in its entirety. The resultant matrix of cultures are designated “candidate cultures” (CCs) as shown in FIG. 32A). These CC lines are heterogeneous in nature, though due to the specific conditions employed in their differentiation, they are enriched in particular cell types and they can be expanded in culture and cryopreserved although their stability and uniformity over time were not studied. Each of these candidate cultures were subsequently plated at clonal densities in an array of different cell culture media optimized for various stromal and epithelial cell types (FIG. 32B). This two-step technique when expanded to a large number of conditions exposes hES-derived cells to a very large number of combinations of conditions to capture cell lines without a previous understanding of the culture needs of any one of the line. The final culture plates were left undisturbed for 14 days in 5% ambient oxygen and a total of 1090 robust colonies resulting from the combinations of conditions that appeared single cell-derived were removed with cloning cylinders and expanded (FIG. 33). The conditions under which each cell line was derived is summarized in Supplementary Table I from West et al., 2008, Regenerative Medicine vol. 3(3) pp. 287-308, which is incorporated by reference herein in its entirety. Cells that did not display a uniform circular morphology or were too closely approximated to neighboring colonies were not selected for propagation (FIG. 33B), and visibly-distinct colonies were required for selection with a minimum separation similar to that of FIG. 33C. As can be seen in FIG. 33D-E, the original colonies frequently showed highly mitotic and uniform populations of cells. A total of 280 lines (25.7%) expanded to at least four roller bottles and of these, approximately 80% cryopreserved/thawed well (judged by the ability to be cryopreserved, thawed, and subsequently expanded at a propagation rate similar to the cells before freezing). Such cells were considered cell lines and assigned ACTC numbers (see Supplementary Table I from West et al., 2008, Regenerative Medicine vol. 3(3) pp. 287-308, which is incorporated by reference herein in its entirety).

### Gene Expression Analysis

To reduce variations in gene expression due to cell cycle artifacts, and to capture an early gene expression profile of the cells, upon being expanded to six well plates, cells were placed in media with a 10-fold reduction in serum or similar growth supplements for five days and all were re-fed two days prior to harvest to reduce feeding artifacts. cDNA from each cell line was hybridized to microarrays for gene expression analysis. cDNA from 242 cell lines (including three biological replicates for C4ELSR2, two biological replicates for the parental hES cell line 119, two technical replicates of X2.2, and two technical replicates of Z11 give a total of 242+9=251 arrays.

cDNA was hybridized to either Illumina microbead arrays (H6V1 and H8V1) (Illumina 1), Illumina H6V2 (Illumina 2), or Affymetrix U133 Plus 2.0 (Affymetrix) and quantile normalized relative fluorescence units (RFUs) are shown in Supplementary Tables II-IV from West et al., 2008, Regenerative Medicine vol. 3(3) pp. 287-308, which is incorporated by reference herein in its entirety. Included in the Illumina 1 data are results using the following controls from fully differentiated cell types: total brain RNA, human foreskin fibroblasts (Xgene) at passage 1 and 5, purified CD34+ and CD133+ peripheral blood lymphocytes and H9 ES cell RNA. Average background signal was 140 RFU and 84 on the Illumina 1 and 2 platforms respectively and 9 on the Affymetrix arrays. Signal was considered positive if >200 RFU on the Illumina 1 and 2 platforms respectively and >100 on the Affymetrix arrays (based on none of the background control probes showing RFU values greater or equal to these numbers). Since only 49 samples were analyzed by Affymetrix arrays, and such data could not be normalized to the Illumina samples, the Affymetrix data is shown in Supplementary Table IV and generally not discussed in this report [see West et al., 2008, Regenerative Medicine vol. 3(3) pp. 287-308, which is incorporated by reference herein in its entirety]. The large number of cell lines made replicate microarray analysis economically unfeasible, therefore select microarray gene expression levels were compared to that obtained by qPCR demonstrating the probably reliability of the data (Supplementary Table I from West et al., 2008, Regenerative Medicine vol. 3(3) pp. 287-308, which is incorporated by reference herein in its entirety) and select cell lines were routinely repeated as technical replicates wherein the original RNA isolate was subjected to repeat microarray analysis, and biological replicates where the cell line was thawed, grown, RNA isolated and microarray analysis repeated, often by differing microarray core facilities and on different chips. Representative replicates included in this report are biological replicates repeated on the same chips of the parental hES cell line H9 (WA Biol and Bio2), three biological replicates of the hEP cell lines C4ELSR2 (Bio 1-3), two technical replicates of X2.2, two technical replicates of Z11 RAPEND17 (Bio 1 being performed on Illumina 1 and Bio 2 on Affymetrix), and other technical replicates of the hEP cell lines 2-2 (Rep 1-2), Z11 (Rep 1-2), RASKEL18 (Rep 1 being performed on Illumina 1 and Rep 2 performed on Affymetrix), and W8 (Rep 1 being performed on Illumina 1 and Rep 2 on Affymetrix) (See Supplementary Tables I-IV from West et al., 2008, Regenerative Medicine vol. 3(3) pp. 287-308, which are incorporated by reference herein in their entirety). Other biological and technical replicates were performed as a quality control showing similar evidence of reproducibility (data not shown).

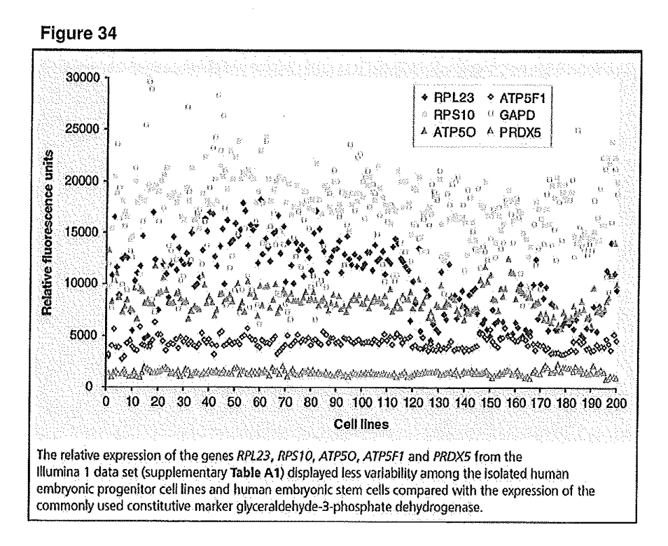

Having obtained gene expression data on so many clonal hES-derived cell lines allowed an unusual opportunity to determine what genes best controls for constitutive expression in both hES cells and their differentiated progeny. Often such data are normalized to the expression of a housekeeping gene such as glyceraldehyde-3-phosphate dehydrogenase (GAPD), however GAPD was never tested against in the context of large arrays and in the breadth of cell types derived in vitro from hES cells. We therefore sorted for genes with the least variation/RFU ratios (quantified as the standard deviation of RFU values/mean RFU values) and identified 5 candidate genes from the Illumina 1 data that display better constitutive expression when compared to GAPD (FIG. 34). It can be seen that while GAPD showed an SD/RFU value of 0.32, the ribosomal component genes RPL23 (SD/RFU of 0.12), and RPS10 (SD/RFU of 0.12), the ATP synthase subunits ATP50 (SD/RFU of 0.14) and ATP5F1 (SD/RFU of 0.13), and the antioxidant enzyme PRDX5 (SD/RFU of 0.14) all were better constitutive markers for hEP cell lines.

### Clonal hEP Cells do not Display hES Markers but Instead Show Markers of Diverse Primitive Embryonic Progenitors

To determine nature and diversity of gene expression in the cultured hEP cell lines, genes in Supplementary Tables II-IV (from West et al., 2008, Regenerative Medicine vol. 3(3) pp. 287-308, which are incorporated by reference herein in their entirety) are rank ordered with genes with the largest RFU value/mean RFU value in all the hEP clones being at the top (high pop analysis) and the horizontal order of the cell lines reflects a hierarchical cluster order (i.e. cells with a similar pattern of gene expression are clustered together). Markers that are relatively highly expressed in each cell line compared to the other lines were determined by rank ordering the ratios of RFU values for each gene in that cell line/average RFU value of that gene for all cell lines (Supplementary Table V from West et al., 2008, Regenerative Medicine vol. 3(3) pp. 287-308, which is incorporated by reference herein in its entirety).

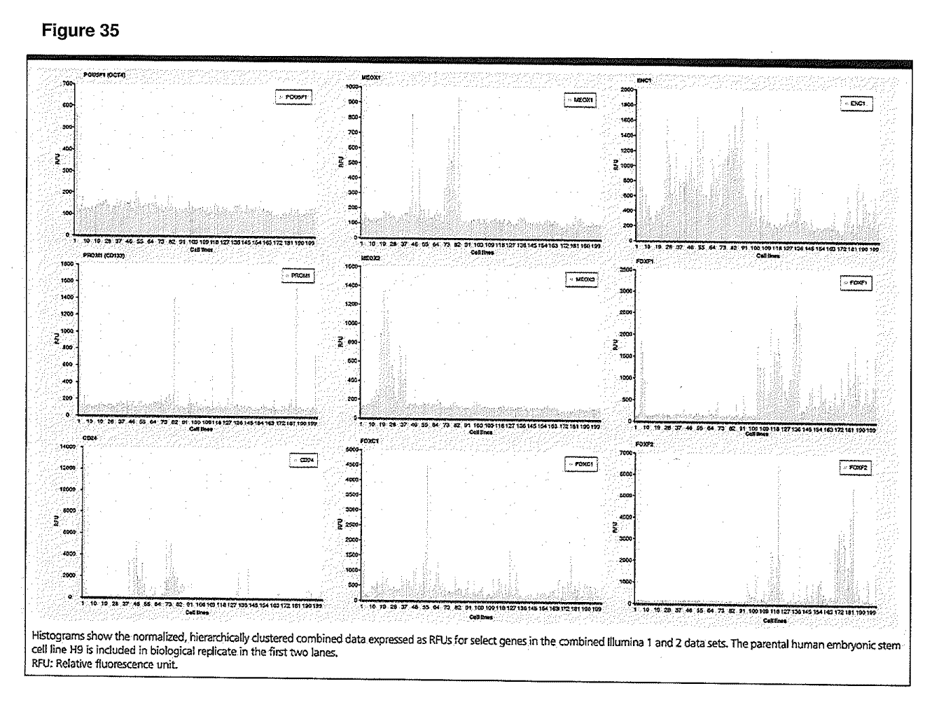

The Illumina 1 and 2 datasets were merged and hierarchically clustered based on sequences the two arrays had in common. Consistent with the cell lines appearing to be at least partially differentiated (i.e. not morphologically similar to the compacted colonies of hES cell lines), as shown in FIG. 35, the EP lines appeared to lack markers of hES cells such as OCT4, though some of the lines expressed markers often associated with stem cells such as CD133, and CD24. In addition, the majority of hEP cell clones expressed markers well known in mouse embryology to be important regulators of cell fate and expressed mainly in embryonic progenitors as opposed to fully differentiated tissues. For example, hierarchical clusters of cell lines expressed relatively high levels of MEOX 1 and MEOX2, are reported to be expressed in early embryonic mesoderm and neural crest derivatives 13,14. The winged helix family of homeobox-containing factors are important in cell fate determination, pattern formation, and organogenesis. Similarly, the winged helix factors such as FOXF1 is mainly expressed in a subset of developing fetal mesodermal cells in the mouse15 is also expressed in various subsets of the hEP cell clones. A total of 136 of 192 (71%) expression results in Illumina 1 data (Supplementary Table II from West et al., 2008, Regenerative Medicine vol. 3(3) pp. 287-308, which is incorporated by reference herein in its entirety) showed RFU values >200 (positive expression) for one of the three embryonic progenitor markers MEOX1, MEOX2, or FOXF1, whereas none of the adult-derived brain, dermal fibroblast, lymphocyte, or hES cell line samples studied expressed the genes. Additional embryonic markers such as the winged helix factor FOXC1 that is reported to be expressed in cranial neural crest, paraxial mesoderm, and somitomeres in the mouse but not adult tissues16 was also highly expressed in numerous hEP cell clones. The gene for the ectoderm-neural cortex protein ENC1 which is mostly expressed in mouse neuroectodermal fated epiblast and brain, and to a lesser extent in some embryonic tissues such as brain, kidney, lung, heart, and liver but exhibits diminished expression in the adult mammal17,18 is similarly expressed in a subset of the clones. Other examples of embryo-specific genes expressed in the lines can be seen in Supplementary Tables II-V (from West et al., 2008, Regenerative Medicine vol. 3(3) pp. 287-308, which are incorporated by reference herein in their entirety) including the relatively high expression of LHX8 in the cell line X7PEND16 (ACTC273) that is reported to be expressed only in the medical ganglionic eminence and perioral mesenchyme of the mouse in the middle embryonic to early postnatal development19, ROR2 which is expressed in the mouse embryo but downregulated in the adult20, SHOX2 which is expressed in embryonic CNS, cranial-facial mesenchyme, heart, and limb mesoderm21, and GPC2, an integral membrane HSPG, is expressed in immature neurons and subsequent to axon formation and terminal differentiation, expression is down-regulated22 as well as other embryo-specific genes (data not shown). Evidence of the potential pluripotency of the clones is seen in the presence of markers of numerous differentiated cell types in some of the lines such as the expression of the neural GFAP, OLIG2, and neuronal markers (E68 [ACTC207]).

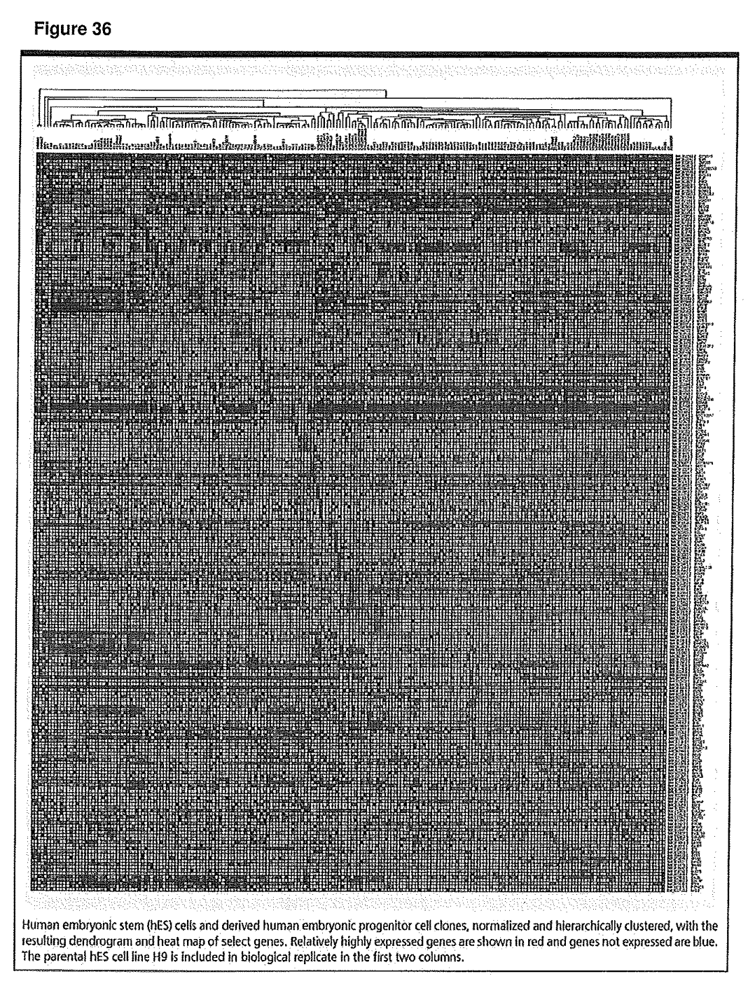

The combined data from Illumina 1 and 2 were subjected to hierarchical clustering and the resulting dendrogram and an abbreviated heat map is shown in FIG. 36 (see also Supplementary Figure A3, from West et al., 2008, Regenerative Medicine vol. 3(3) pp. 287-308, which is incorporated by reference herein in its entirety). As seen in FIG. 36, genes that are expressed in relatively high levels are coded red and low levels of expression are blue. It can be seen that biological replicates of the human ES cell line H9 (WA09) clustered together and showed relatively high levels of CYP26A1, a P450 retinoic acid-inactivating enzyme that while reported to play an important role in anterior-posterior positioning in the gastrulating embryo, has not been reported to be expressed at such high levels in cultured ES cells23. The ES cells, but not the differentiated cell clones also expressed EBAF (lefty2 in the mouse) an inhibitor of nodal and reported to be rapidly down-regulated following hES cell differentiation24, as well as the transcription factors ZNF206 and ZIC3, both reported to be expressed at relatively high levels in hES cells but downregulated during differentiation and to play a role in maintaining an undifferentiated state25,26. It can be seen in FIG. 36 and Supplementary Figure A3 (from West et al., 2008, Regenerative Medicine vol. 3(3) pp. 287-308, which is incorporated by reference herein in its entirety) that there are similar patterns of gene expression in the other biological and technical replicates but a wide array of different differentiated markers among the hEP cell lines. Examples include the genes PLP1, PMP2, GRIN1, and GABRA1 typically expressed in neuroglial cells and highly expressed in the line E68 (ACTC). Other examples are the gene Myosin Va which is involved in the transport of secretory vesicles of neurons and melanocytes27, GARP which is expressed at relatively high levels during murine embryogenesis such as in limb dermis, smooth muscle, and vascular endothelial cells28, EDIL3 (developmentally-regulated endothelial locus-1) which is reported to be involved in the embryonic regulation of vascular morphogenesis29, Col24A1 which is relatively specific to developing bone & cornea30, and SEMA5A which is expressed by oligodendrocytes31. Other selected markers for other lines are shown in FIG. 36 and Supplementary Figure A3 (from West et al., 2008, Regenerative Medicine vol. 3(3) pp. 287-308, which is incorporated by reference herein in its entirety). The expression of these markers, while not definitively diagnostic of the cell types discussed, nevertheless provides evidence of the diversity of cell types that can be propagated clonally from hES cell lines in vitro.

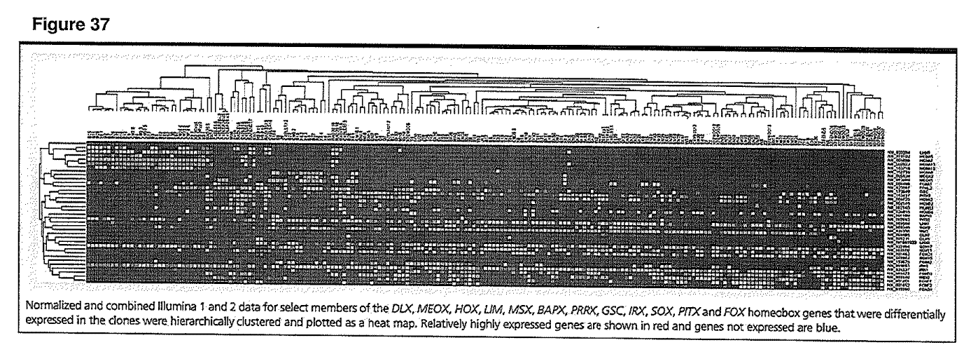

The diversity of clonal derivatives can also be seen through the specific expression of homeobox genes. All differentiated cells, like reports of dermal fibroblasts32 have the potential to vary widely in gene expression from one geographic location in the body to another depending on DLX, MEOX, HOX, LIM, MSX, BAPX, PRRX, GSC, IRX, SOX, PITX, and FOX gene expression. As can be seen in FIG. 37, there is a diversity of homeobox gene expression in the hEP cell lines perhaps reflecting the fact that while there are multiple isolates of lateral plate mesoderm, differences in HOX gene expression are resulting in subtle differences in extracellular matrix and other proteins that lead to the cells being grouped as unique cell types.

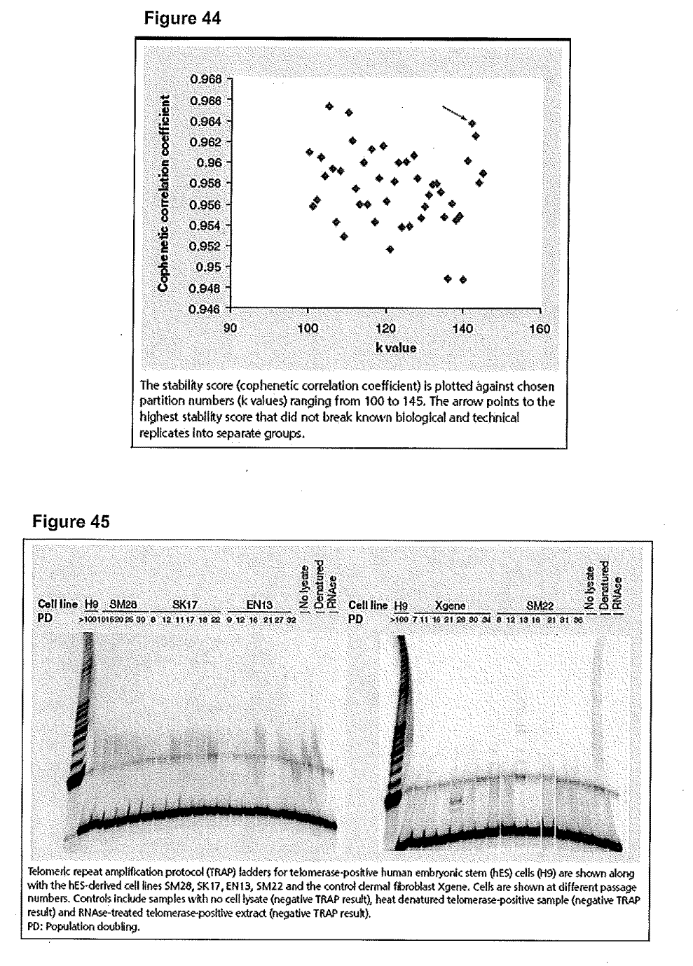

To provide an objective measure of the complexity of the hEP cell library, a grouping using NMF analysis was performed. The k-value was incrementally altered to obtain the highest stability score without scattering known biological replicates (three independent isolations of ELSR2, two biological replicates of H9, and two technical replicates of Z11). The stability scores where k values range from 100-145 are shown in FIG. 44 and the resulting NMF plot is shown in FIG. 38. The cells were assigned group numbers and these group numbers as well as the order in which the cells are displayed in the NMF plot are shown in Supplementary Table I (from West et al., 2008, Regenerative Medicine vol. 3(3) pp. 287-308, which is incorporated by reference herein in its entirety). The most stable k-value was 140 suggesting that the complexity of the cell lines analyzed on the Illumina platform was 140. Consistent with this conclusion, the cells within a given group have similar marker genes and cluster together (FIG. 36). For example, the cells of group 30 (E84, E30, E3, E73, E57, and E67) all have a similar pattern of gene expression markers such as S100A4 (Supplementary Table V from West et al., 2008, Regenerative Medicine vol. 3(3) pp. 287-308, which is incorporated by reference herein in its entirety) and cluster as a discrete group by hierarchical clustering (FIG. 36). Also, the NMF analysis did not split biological or technical replicates. The cell lines analyzed with Affymetrix arrays could not be combined with those lines analyzed with Illumina arrays in the NMF analysis, therefore the estimated complexity is restricted to those cell lines assayed on the Illumina platform. However, because at least one line (MEL2, ACTC) analyzed on the Affymetrix arrays displays numerous unique markers not seen in any cell line analyzed on Illumina bead arrays, but it appear to include cell lines with markers not characterized on the Illumina platform, we conclude that the number of distinguishable hEP cell cultures isolated and described in Supplementary Table I (from West et al., 2008, Regenerative Medicine vol. 3(3) pp. 287-308, which is incorporated by reference herein in its entirety) were >140.

### Immunocytochemical Confirmation of hEP Microarray Gene Expression Analysis

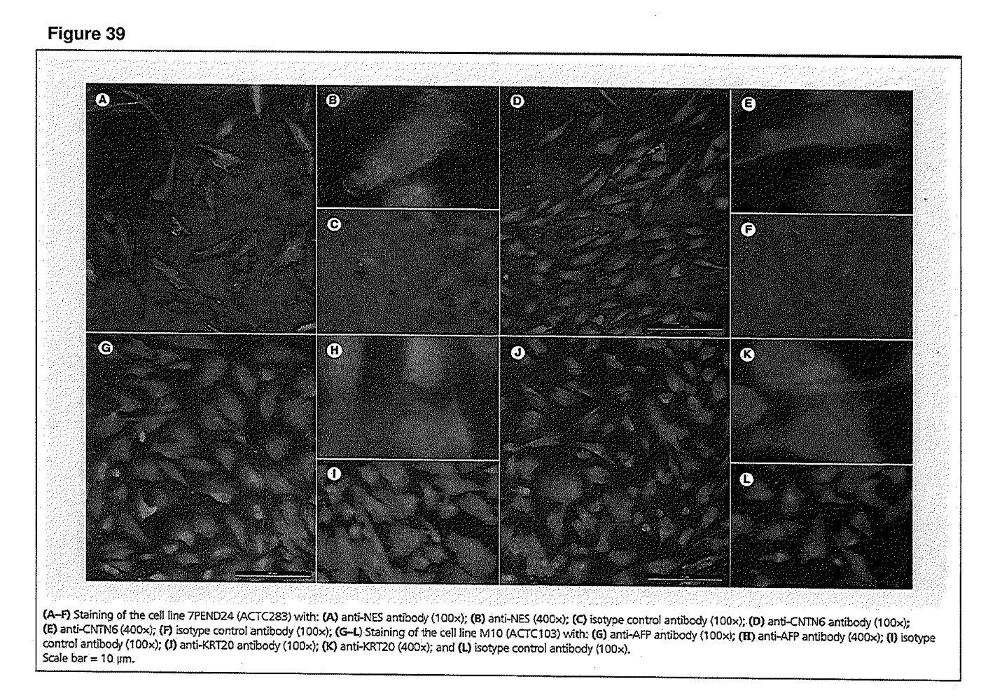

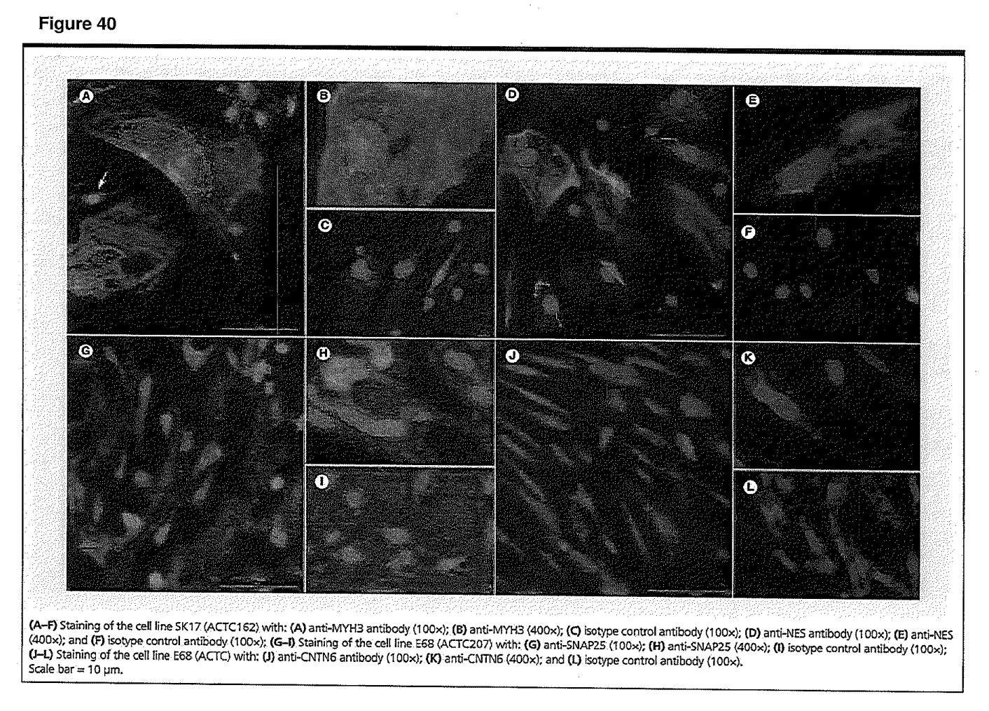

The microarray gene expression data suggested that the hEP cell lines express profiles of numerous primitive neural crest, endodermal, mesodermal, or ectodermal lineages. To determine whether protein expression of several unique markers of differentiation correlated with the relatively high RNA expression levels of the markers in hEP cell lines, we used immunocytochemical analysis. In each of 4 hEP cell lines tested, proteins corresponding to highly expressed mRNAs were readily detected by immunocytochemical staining with the appropriate antibody (FIGS. 39 and 40). Accordingly, the cell line 7PEND24 (ACTC283) expressed genes consistent with being a neural crest line such as the melanocyte markers TYRP1 and EDNRB, peripheral neuron markers such as EGR2, STMN2, DCX, CNTNAP2, GPC2, and PROM1, and cartilage markers such as CILP (See Supplementary Table V from West et al., 2008, Regenerative Medicine vol. 3(3) pp. 287-308, which is incorporated by reference herein in its entirety). The neural progenitor markers nestin (NES)33 and contactin 6 (CNTN6)34 were confirmed on a protein level with specific antibodies in the cell line corresponding with mRNA expression (FIG. 39; a-f). A typical intermediate filament staining pattern for NES was observed under high power (FIG. 39b). In the case of the cell line 7PEND24, the most caudal HOX gene expression was HOXA2, HOXB2, suggesting it corresponded to an origin in the hindbrain.

The cell line M10 (ACTC103) expressed relatively high levels of FOXA2, TCF2(HNF1B), and normal mucosa of esophagus-specific 1 (NMES1) (See Supplementary Table V from West et al., 2008, Regenerative Medicine vol. 3(3) pp. 287-308, which is incorporated by reference herein in its entirety) consistent with the cells being endodermal, possibly oral or esophageal epithelia in nature35-37. The genes alpha-fetoprotein (AFP) and keratin 20 (KRT20)38 were also expressed at relatively high levels and the corresponding proteins were confirmed to also be expressed using specific antibodies (FIG. 39; g-l). A typical keratin filament staining pattern was observed under high power (FIG. 39k). The most caudal HOX gene expression was HOXB5 suggesting that the cell line is foregut in nature.

The mesodermal marker myosin heavy chain 3 (MYH3) and intermediate filament nestin (NES) both of which are known to be expressed in embryonic but not adult heart and skeletal muscle39,40 were detected in the SK17 (ACTC162) cell line which expressed both proteins at detectable levels (FIG. 40; a-f). The MYH3 staining of SK17 resulted in a staining pattern with myocyte-like microfilament morphology (FIG. 40; a-b). The cells also expressed relatively high levels of ACTC, MYBPH, TNNC1, MYOD1, HUMMLC2B (See Supplementary Table VI from West et al., 2008, Regenerative Medicine vol. 3(3) pp. 287-308, which is incorporated by reference herein in its entirety) and most caudal HOX gene expression was HOXA11, HOXB9, and HOXC6. Only the large cells stained positive for MYH3, suggestive of a more primitive cell type in the cultures as well. Interestingly, SK17 also expressed cardiac myosin heavy chain MYH7 and markers normally associated with cardiac cells such as CASQ2, TNNT2, neuronal cell types such as NEF3, and axon guidance molecules such as SPON1, SLIT2, and RTN4 (Supplementary Table V from West et al., 2008, Regenerative Medicine vol. 3(3) pp. 287-308, which is incorporated by reference herein in its entirety). The expression of these neuronal markers and the unique and strong expression of MYBPH which is expressed in skeletal and heart conduction fibers and SLN which is expressed in soleus and artial but not ventricular cardiac muscle, suggests these cells may be a previously unrecognized cardiac progenitor perhaps playing a role in the conduction system of the heart.

As previously discussed, the cell line E68 (ACTC207) expressed numerous gene expression markers of neuroglial lineages but lacked HOX gene expression. The ectodermal markers synaptosomal associated protein 25 (SNAP 25) and contactin 6 (CTNTN6) were detected on a protein level in the E68 cell line that expressed both high levels of both marker mRNAs (FIG. 40; g-l). For the detection of each of the previously-discussed marker proteins, substitution of the primary antibody with an isotype matched control antibody resulted in little or no detection of fluorescent secondary antibody binding (FIG. 39; c,f,i,l and FIG. 40; c,f,i, l). Overall, protein markers of differentiation were appropriately expressed in those hEP lines that over-expressed the corresponding marker gene.

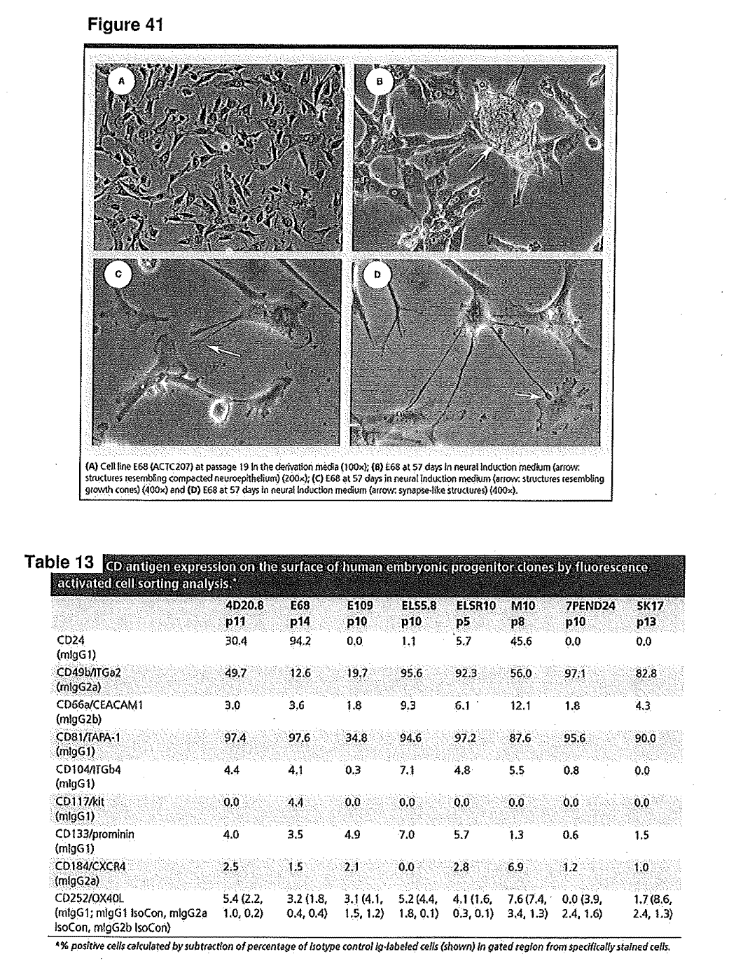

The transfer of E68 to neurobasal medium supplemented with N2 for 57 days, altered the proliferative population of stellate cells FIG. 41A, to cells with a more neuroglial morphology, including clusters of mutually adherent cells resembling neurospheres (FIG. 41B), cells displaying growth cone-like structures (FIG. 41C), and cells with structures resembling synapses (FIG. 41D) consistent with the immunocytochemical markers shown for E68 in FIG. 40 (g-l) and the gene expression markers observed in the line (Supplementary Table V from West et al., 2008, Regenerative Medicine vol. 3(3) pp. 287-308, which is incorporated by reference herein in its entirety), though further physiological studies of the cells to confirm neuron-like activity is warranted.

### Clonal hEP Lines Express Diverse Cell Surface Antigen Expression

The use of affinity methods to purify cell lineages has often been used in blood cell therapy. We therefore investigated whether hEP cell lines that showed differentially-expressed CD antigens predicted the presence of these antigens on the cell surface, potentially facilitating the repeated isolation of desired clones. As seen in Supplementary Table VI (from West et al., 2008, Regenerative Medicine vol. 3(3) pp. 287-308, which is incorporated by reference herein in its entirety), CD antigen gene expression varied widely among the cell lines. We then compared the percent positive cells as determined by flow cytometry to the expression of selected CD antigens in a subset of the cell lines. By gene expression, CD81 was strongly expressed in all the lines and as seen in Table 2, all cell lines were positive for this antigen. In contrast, CD24 gene expression in 4D20.8 (ACTC84) was weakly positive, E68 (ACTC207) was strongly positive, E109 (ACTC117) was negative, ELS5.8 (ACTC238) was negative, ELSR10 (ACTC152) was negative, M10 (ACTC103) was negative, 7PEND24 (ACTC283) was negative, and SK17 (ACTC162) was positive. As seen in Table 2, 30.4% of 4D20.8, 94.2% of E68, and 45.6% of M10 cells were positive, but the other lines were negative. Interestingly, the CD24 antigen distinguished the hindbrain neural crest neural progenitor line 7PEND24 (CD24−) from the HOX-neural progenitor line E68 (CD24+) demonstrating the usefulness of clonally isolated hEP lines in potentially identifying useful cell surface antigens. The variability of expression of CD antigens in differentiated hEP cell lines may be a result of continued differentiation of the cells subsequent to clonal isolation and underscores the need for additional study.

hEP Clones express unique secreted factors

Embryonic cells express a host of secreted factors that regulate complex organogenesis. We profiled those genes known to be processed as secreted proteins and those genes differentially expressed in each line are summarized in Supplementary Table VII (from West et al., 2008, Regenerative Medicine vol. 3(3) pp. 287-308, which is incorporated by reference herein in its entirety). It can be seen that the isolated hEP cell clones show expression of a wide array of transcripts for growth factors, cytokines, proteases, protease inhibitors, and extracellular matrix factors. We then selected an arbitrary subset of the lines and performed ELISA to determine whether we could confirm protein expression in the conditioned medium. Gene expression profile data suggests that the cell lines EN 13 and EN 47 are expressing amphiregulin (AREG) in measurable amounts whereas the cell lines SK 17 and Xgene fibroblasts express very little or no AREG. This observation is validated on a protein level as seen in Supplementary Table VIII (from West et al., 2008, Regenerative Medicine vol. 3(3) pp. 287-308, which is incorporated by reference herein in its entirety) where the lines EN13 and EN47 showed 6.35 ng/ml and 6.36 ng/ml respectively in 72 hour conditioned medium and SK17 and Xgene were negative. Similarly, gene expression profile data also suggests that the cell line ELSR10 may be secreting the following factors: FGF-7, IGFBP-5, PDGF-BB, TGFb-1, TIMP-1 and Vitronectin. Since some of the factors may be secreted in small amounts, below the detection level, the cell culture medium was concentrated 5 fold using a Millipore Ultrafree concentrator (Thermo Fisher Cat # UFV5 BCC 25) with a 5,000 MW cutoff. Medium from the cell lines EN 13, EN 47, SK 17 and Xgene fibroblasts were tested simultaneously for the same factors. Results shown in Supplementary Table VIII (from West et al., 2008, Regenerative Medicine vol. 3(3) pp. 287-308, which is incorporated by reference herein in its entirety) also validate the gene expression levels in that the cell line ELSR10 alone expressed high levels of all these factors relative to the nonexpressing cell lines.

### hEP Cells Lack Tumorigenicity

While hES cells generate benign teratomas when injected into immunocompromised animals, the tumorigenicity of purified hEP types has not been extensively studied. The examination of genes expressed at relatively high levels in each line revealed numerous genes known primarily for their expression in malignancies and in embryonic development (oncofetal genes). For example, SILV is reportedly expressed in a large number of melanomas41 and in embryonic retinal pigment epithelium and neural crest-derived melanoblasts42 and is expressed at relatively high levels in SK17 (ACTC162). Other such oncofetal genes expressed in the isolated hEP cell lines include PLAG1, AMIGO2, HCLS1, SPINK1, PRAME, INSM1, RAGE, ENC1, BCAS1, GRM1, TSGA10, S100A2, A4, and A6, GPC3, EGFL6, PSG5, CEACAM1, CGPC3, SRPUL, DCDC2, LRRN5, SOX11, RUNX3, CA12, STARD10, CXCL1, ANPEP, GAGE6, NCOA6, TACSTD2, and TSPAN8. We therefore tested the tumorigenicity of an arbitrary group of the hEP cell lines in SCID mice. 20 million cells from each of the cell lines B16 (ACTC59), B28 (ACTC60), 6-1 (ACTC64), B26 (ACTC50), B11 (ACTC58), B2 (ACTC51), CM02 (ACTC77), E75 (ACTC102), E15 (ACTC98), 4D20.9 (ACTC82), E72 (ACTC100), EN7 (ACTC184), EN55 (ACTC185), SKIT (ACTC162), and Z11 (ACTC194) were injected (each cell line injected into 2 SCID mice with approximately 10 million cells/mouse or a total of 30 mice and 60 injection sites). Half the cells (5 million) were injected intramuscularly into the right rear leg and the other 5 million subcutaneously into the left rear leg. After 4-6 months, a thorough pathological analysis could reveal no grossly visible abnormalities, dehydration, malnutrition, lesions, hair loss, inflammation or any other evidence of past or current disease process and upon dissection, there was no evidence of tumors, congregation, redness, necrosis, or edema in the limbs, abdomen, thoracic cavity, neck. One exception was the cell line B28 which showed an approximately 1 mm nodule between the skin and leg muscle near the site of injection. In our experience, the injection of similar numbers of hES cells at these sites and for these periods of time would have led to teratoma formation in the majority of animals.

hEP Cells Include Clones with a Robust and Mortal Proliferative Capacity

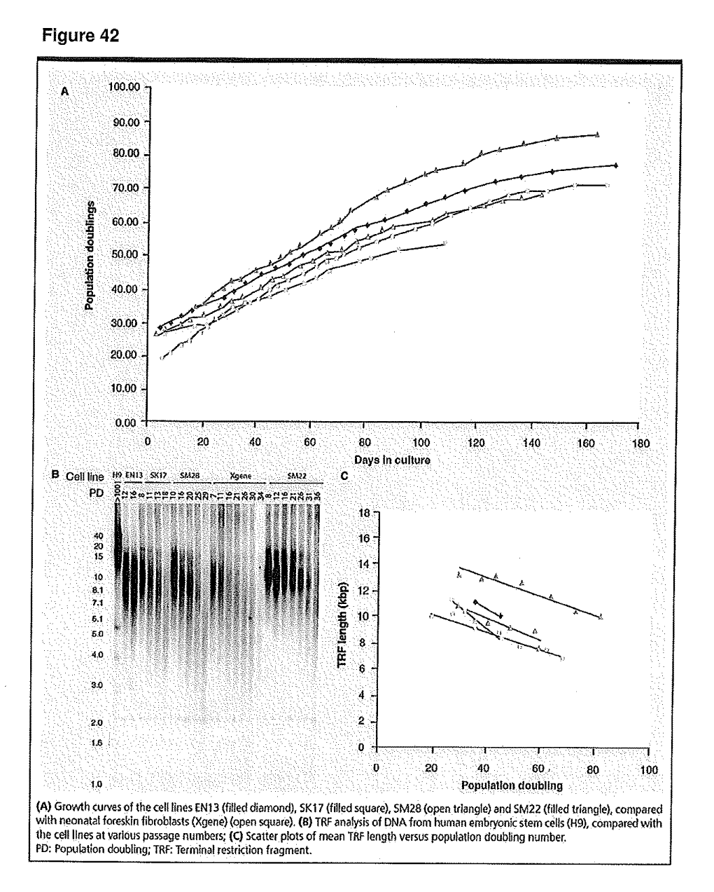

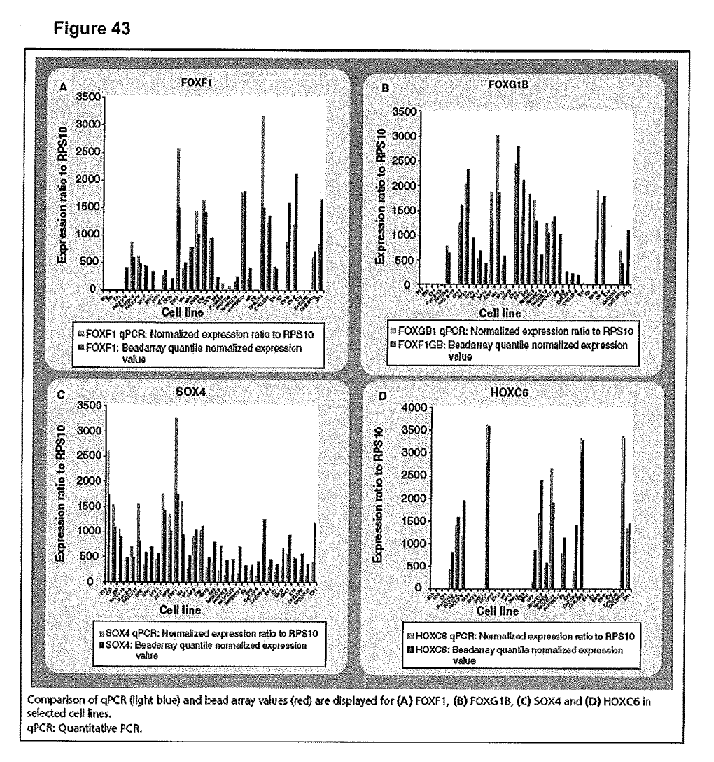

Human germ-line cells such as sperm show relatively long and stable mean telomere restriction fragment lengths of 12-15kbp43. Human ES cells are likely unique among cultured normal human cells in maintaining germ-line telomere length through the activity of telomerase1. We therefore assayed selected early hES-derived hEP cell clones for telomere length by Southern analysis and telomerase activity by the TRAP assay during extended passaging in vitro to provide insight into the proliferation potential of the lines compared to normal human cells of a neonatal origin. As shown in FIG. 42A, the lines EN13, SK17, SM28 and SM22 were propagated and compared to a neonatal foreskin fibroblast cell line Xgene. With the exception of the line SK17, all clonal hEP cell lines showed equal or greater proliferative capacity than the non-clonal neonatal foreskin fibroblasts. Since the majority of human cell clones generally senesce 20 or more doublings earlier than the mass culture from which they were derived (i.e. mass cultures proliferate to the limit of the longest lived constituent clone), and most human cell clones isolated from neonatal or adult sources senesce in less than 50 PD, we conclude that hEP cell clones studied herein may markedly exceed the proliferative capacity of cells derived from neonatal or adult sources. As shown in FIG. 42B, a Southern blot of telomere lengths of the parental hES cell line H9, versus hEP cell clones isolated from that line shows that telomere length is germ-line in length in the line H9 and subsequently shortens in all hEP cell clones studied. As shown in FIG. 42C, the initial telomere lengths appears to be higher in the cell clones in the earliest passages studied despite being clonally isolated, and the mean rate of loss was comparable in the lines with the exception of SK17 which showed an accelerated loss, likely due to poor plating efficiency and/or apoptosis (data not shown). Telomerase activity was high in the hES cell line H9, but low or negative in all hEP cell lines at all passages measured (FIG. 45).

### Discussion

We describe a simple combinatorial protocol that, like the shotgun cloning of genes, allows the nonspecific generation of a library of cell lines that can later be analyzed and collated using microarray and bioinformatics analysis. Surprisingly, many of the lines are capable of expansion in standard adherent culture and appear to display a wide array of markers of embryonic progenitor cell types from endodermal, mesodermal, ectodermal, and neural crest lineages. The presence of diverse but discrete homeobox gene expression in these lines is consistent with the wide variety of homeobox gene expression patterns observed even in homologous cell types such as dermal fibroblasts isolated from various regions of the body32 and suggests that the clonal isolation may have occurred subsequent to the activation of these homeobox genes, though the uniformity of these transcription factors in the clones was not assayed in this study. It should be noted that only a small field of combinations of differentiation conditions, differentiation times, and subsequent clonal propagation medium were used in this study. Therefore, it is possible that further efforts to expand the conditions may yield additional cell types. It should also be noted that the variation of media used in propagating the lines may have been a source of variability in gene expression, and that some degree the diversity observed may be due to the influence of the media, whereas the differentiated state of such cells would otherwise be identical. Further studies are warranted to study these effects.

A study of this scale required that individual assays, such as qPCR to confirm the microarray results, ELISA to measure immunoreactive secreted proteins, immunocytochemistry to confirm protein expression in situ, or telomere assays could only be performed on a small subset of the cell lines. Therefore, further study of the cell lines is required to interpret the gene expression profiles reported. The ability to scale and cryopreserve many diverse hEP cell lines may allow the cells to be distributed and thereby help standardize studies in stem cell biology. The robust proliferative capacity of many of the clones likely reflects the fact that they were recently isolated from hES cells that typically show germ-line telomere length (i.e. approximately 15 kbp TRF length). These unusually long telomeres give hEP cell lines a benefit compared to fetal or adult-derived cells that typically have far shorter telomeres and because they are terminally differentiated do not propagate in vitro. The scalability of hES cell lines may therefore provide a useful point of scalability other than the scaling of hES cell lines themselves. Our initial profiling of hEP cell clones is necessarily limited and preliminary due to the large number of cell lines isolated and the fact that some of the cells were analyzed on the Affymetrix microarray platform and could not be normalized with the cell lines analyzed by Illumina microarrays. Much additional study needs to be performed on the differentiation potential and stability of the lines after being passaged in vitro. The presented data suggests that cloned libraries of hES-derived progenitor lines may provide a useful means of profiling the gene expression profile of primitive cell types in order to identify their differentiation potential, cell surface antigens including growth factor receptors, and secreted proteins such as growth factors and cytokines. The potential of such cells for use in therapy awaits definition of the developmental potential of the cell lines and studies of the survival and function of such primitive cells in normal or pathological adult tissue (heterochronic transplantation). Because these lines could easily be documented by photomicroscopy to have a differentiated morphology when originally plated as a single cell, clonal propagation may provide a useful means of insuring the absence of contaminating hES cells in formulations or other cell types that could lead to tumor formation or the differentiation of undesired cell types.

The prospect of generating larger libraries of hEP cell clones and the complex and poorly characterized markers for early human embryonic lineages with a complexity that likely exceeds 103, highlights the need to database the markers and cell surface antigens of the early lineages of the human developmental tree44. Such a database, and a large library of defined cell lines may facilitate the translation of the developmental potential of hES cells into actual cell therapies.

### Methods

hES cell culture and generation of candidate cultures. The hES cell lines used in this study were previously described H9 (National Institutes of Health-registered as WA09) and the line (MA03) derived at Advanced Cell Technology. hES cells were routinely cultured in hES medium (KO-DMEM (Invitrogen, Carlsbad, Calif.), 1× nonessential amino acids (Invitrogen, Carlsbad, Calif.), 1× Glutamax-1 (Invitrogen, Carlsbad, Calif.), 55 uM beta-mercaptoethanol (Invitrogen, Carlsbad, Calif.), 8% Knock-Out Serum Replacement (Invitrogen, Carlsbad, Calif.), 8% Plasmanate, 10 ng/ml LIF (Millipore, Billerica, Mass.), 4 ng/ml bFGF (Millipore, Billerica, Mass.), 50 unit/ml Penicillin-50 units/ml Streptomycin (Invitrogen, Carlsbad, Calif.). The cells lines are maintained in and all subsequent experiments are carried out at 37° C. in an atmosphere of 10% CO2 and 5% O2 on Mitomycin-C treated mouse embryonic fibroblasts (MEFs) and passaged by trypsinization. hES cells were plated at 500-10,000 cells per 15 cm dish. Candidate culture differentiation experiments were performed with either adherent hES cells grown on MEFs or with hES embryoid bodies (EB). For adherent differentiation experiments, hES cells were allowed to grow to confluence and differentiated by a variety of methods described in Supplementary Table I (from West et al., 2008, Regenerative Medicine vol. 3(3) pp. 287-308, which is incorporated by reference herein in its entirety). For example, in the case of colony in situ differentiation in DMEM with 10% FCS, growth medium was replaced with DMEM medium containing 10% FBS for differentiation and after various time periods (1, 2, 3, 4, 5, 7, and 9 days in differentiation medium), the cells are dissociated with 0.25% trypsin (Invitrogen, Carlsbad, Calif.) and plated in 150 cm2 flasks for expansion. The candidate cells from each time point in the 150 cm2 flasks were plated out for cloning and expansion as described below. For EB differentiation experiments, confluent hES cultures were treated for 15 minutes at 37° C. with 1 mg/ml Collagenase IV (in DMEM, Invitrogen, Carlsbad, Calif.) to release the colonies. The detached, intact colonies were scraped and collected by centrifugation (150×g for 5 minutes), resuspended in differentiation medium described in Table 13 and transferred to a single well of a 6-well Ultra-Low Binding plate (Corning, distributed by Fisher Scientific, Pittsburgh, Pa.) containing the same differentiation medium. The EBs were allowed to differentiate, depending on the experiment, from 4-7 days and the differentiated EBs dissociated with 0.25% trypsin, plated in 6-well plates containing various expansion medium. The candidate cultures in the 6 well plates are allowed to grow to confluence and plated out for cloning and expansion as described below.

Isolation and expansion of clonal cell lines. The differentiated candidate cell cultures described above were dissociated with 0.25% trypsin to single cells and plated onto duplicate 15 cm gelatin coated plates at cloning densities of approximately 500 and/or 1,000 and/or 2,000 and/or 5,000 cells per plate for further differentiation and expansion in a variety of growth media described in Table 13. The clonal density cells were allowed to grow, undisturbed, for 10-14 days and colonies that develop were identified and collected with cloning cylinders and trypsin using standard techniques10a. The cloned colonies were transferred onto gelatin coated 24 well plates for expansion. As the clones become confluent in the 24 well plates, they were sequentially expanded to 12 well, 6 well, T-25 flask, T-75 flask, T-150 or T-225 flasks and, finally, roller bottles. Clonal cell lines that expand to the roller bottle stage are assigned a unique ACTC identification number, photographed and cryopreserved in aliquots for later use. Once cells reached a confluent T-25 flask, they were passaged to a T-75 flask and a fraction of the cells (5×105) were removed for plating in a gelatinized 6 cm dish for gene expression profile analysis. Following removal of the cell clones from the cloning plates, remaining colonies were visualized by Crystal violet staining (Sigma HT9132-1L) in 100% ethanol per manufacturer's instructions. Cell Culture media utilized in experiments and described in text and Table 13: Smooth muscle cell basal medium (Cat# C-22062B) and growth supplement (Cat# C-39267), Skeletal muscle basal medium (Cat# 22060B) and growth supplement (Cat# C-39365), Endothelial cell basal medium (Cat# C-22221) and growth supplement (Cat# C-39221), Melanocyte cell basal medium (Cat# C-24010B) and growth supplement (Cat# C-39415) were obtained from PromoCell GmbH (Heidelberg, Germany). Epi-Life, calcium free/phenol red free medium (Cat# M-EPIcf/PRF-500) and low serum growth supplement (Cat# S-003-10) were purchased from Cascade Biologics (Portland, Oreg.). Mesencult basal medium (Cat#05041) and supplement (Cat#5402) were obtained from Stem Cell Technologies (Vancouver, BC). Dulbecco's modified Eagle's medium (Cat#11960-069) and Fetal bovine serum (Cat# SH30070-03) were purchased from Invitrogen (Carlsbad, Calif.) and Hyclone (Logan, Utah) respectively. Medium and supplements were combined according to manufacturer's instructions.

### Gene Expression Analysis:

Total RNA was extracted directly from cells growing in 6-well or 6 cm tissue culture plates using Qiagen RNeasy mini kits according to the manufacturer's instructions. RNA concentrations were measured using a Beckman DU530 or Nanodrop spectrophotometer and RNA quality determined by denaturing agarose gel electrophoresis or an Agilent 2100 bioanalyzer. Whole-genome expression analysis was carried out using Affymetrix Human Genome U133 Plus 2.0 GeneChip® system, Illumina Human-6 v1 and HumanRef-8 v1 Beadchips (Illumina 1), and Illumina Human-6 v2 Beadchips (Illumina 2), and RNA levels for certain genes were confirmed by quantitative PCR. For Illumina BeadArrays, total RNA was linearly amplified and biotin-labeled using Illumina TotalPrep kits (Ambion), and cRNA was quality controlled using an Agilent 2100 Bioanalyzer. cRNA was hybridized to Illumina BeadChips, processed, and read using a BeadStation array reader according to the manufacturer's instructions (Illumina). For Affymetrix genechip analysis, a two cycle cRNA amplification and labeling was performed. 100 ng of total RNA from each sample was used for the first cycle of double-stranded cDNA synthesis using in vitro transcription (IVT) amplification of cRNA (MEGAscript T7 kit, Ambion,) followed by two-cycles of target labeling (Affymetrix). Labelled cRNA (15 ug) was fragmented and hybridized according to the manufacturer's instructions. Relative Fluorescence Unit (RFU) values for all of the cell lines with common probe sets were quantile normalized. In FIG. 34, variation of the levels of expression of a single gene across cell lines was calculated as the ratio of the standard deviation of RFU values/mean RFU and is reported as the SD/RFU ratio. In Supplementary Tables II-IV (from West et al., 2008, Regenerative Medicine vol. 3(3) pp. 287-308, which are incorporated by reference herein in their entirety) the genes are displayed in rank order (highest-lowest) for the ratio of (highest RFU value observed for the gene in the entire set of cell lines−Average RFU value)/Ave RFU value. In Supplementary Table V (from West et al., 2008, Regenerative Medicine vol. 3(3) pp. 287-308, which is incorporated by reference herein in its entirety) the top 45 differentially expressed genes rank ordered (highest-lowest) for the ratio of (highest RFU value observed for the gene in the individual cell line/Ave RFU value for all cell lines. In Supplementary Table VI (from West et al., 2008, Regenerative Medicine vol. 3(3) pp. 287-308, which is incorporated by reference herein in its entirety) the genes corresponding to recognized CD antigens are displayed in rank order (highest-lowest) and also (lowest to highest) for the ratio of highest RFU value observed for the gene in the entire set of cell lines/Ave RFU value and lowest RFU value observed for the gene in the entire set of cell lines/Ave RFU value respectively. In Supplementary Table VII (from West et al., 2008, Regenerative Medicine vol. 3(3) pp. 287-308, which is incorporated by reference herein in its entirety) the genes corresponding to secreted proteins are displayed in rank order (highest-lowest) for the ratio of highest RFU value observed for the gene in the entire set of cell lines/Ave RFU value.

To validate the expression observed in beadarray and genechip data sets, qPCR was used to independently measure RNA levels for FOXF1, FOXG1B, HOXA10, HOXA5, HOXB2, HOXB7, HOXB8, HOXB9, HOXC6, MYOD1, MYOG, PRDX5, RPL24, SOX11, SOX4 and SOX8 genes in the cell lines cell lines B29, 1330, E51, RAD20-19, RAD20-5, RAD20-16, SK57, SK60, SK61, SK17, SK30, EN31, W4, W10, SM28, EN5, EN13, SK5, RASKEL6, RASKEL8, RASKEL18, W8, RAPEND17, E68, C4ELS5-8, C4ELS5-6, E44, E3, EN18, EN47, E15, C4ELSR2, C4ELSR13 and EN1. RNA used samples used for qPCR were the same as used for gene expression analysis with the Illumina Beadchips or Affymetrix genechips. The cDNA was synthesized with Invitrogen SuperScript III First-Strand Synthesis SuperMix for qRT-PCR and QPCR was performed using a BIORAD iCycler with an iQ5 Multicolor Real-Time PCR Detection System. The reactions used Invitrogen SYBR GreenER qPCR Super Mix for the iCycler.

### NMF Consensus Description:

Gene expression data were analyzed using non-negative matrix factorization (NMF)45. NMF is an unsupervised learning algorithm which identifies molecular patterns when applied to gene expression data by detecting context-dependent patterns of gene expression in complex biological systems46. The NMF analysis was run in GenePattern downloaded from the Broad Institute (http://www.broad.mit.edu/cancer/software/genepattern/) at MIT47. The parameters used for the NMF analysis shown in the NMF Consensus Plot (FIG. 38) were N=3232 most differentially expressed gene; M=202 cell lines. NMF analyses were iteratively calculated with increasing k from 1 to 150 and selected a k=140 based on stability of the calculated co-phenetic coefficient to minimize the divergence norm. The default NMF Consensus settings of number of clusterings=20, number of iterations=2000, stop.convergence=40, stop.frequency=10 33.

Tumorigenicity in Mice. Approximately 20 million cells from each of the cell lines B16, B28, 6-1, B26, B11, B2, CM02, E75, E15, 4D20.9, E72, EN7, EN55, SK17, and Z11 were each injected into 2 SCID mice with approximately (or 10 million cells/mouse). Half the cells (5 million) were injected intramuscularly into the right rear leg and the other 5 million subcutaneously into the left rear leg. After 4-6 months, each mouse was placed supine on the table, and under an operating microscope, bilateral skin incisions were made starting at the knee joint, and extending to the abdomen and then medially to the spine. The skin was then peeled back exposing all the surface leg muscles. The surface of the skin was examined, as well as the muscle surface. The muscles were transected every 2 mm. The femur was exposed and examined. Following bilateral limb dissection and examination, the abdominal incision was extended anteriorly to the thymus gland, exposing all abdominal organs, tissues as well as the lungs and myocardium. Every organ and tissue (thymus gland, heart, lungs, kidneys, adrenal glands, liver, gastrointestinal organs, reproductive tract and the inner lining of the thoracic and abdominal cavity) were examined both on the surface and following transsection, under the operating microscope.

Flow Cytometry Analysis of Cell Surface Antigens. A representative number of cell lines at defined passage (p) numbers (4D20.8, p11; E68, p14; E109, p10; ELS5.8, p10; ELSR10, p15; M10, p8; 7PEND24, p10; SK17, p13) were analyzed by immunostaining for various cell surface antigens and flow cytometry analysis. Adherent cells were detached using ESGRO Complete Accutase (Chemicon/Millipore, Temecula, Calif.) to minimize antigen degradation. Cell aliquots were then incubated with the following standard panel of mouse monoclonal CD antibodies: CD24 (Chemicon, CBL561), CD49b (Southern Biotech, Birmingham, Ala.; 9426-01), CD66a (R&D Systems, Minneapolis, Minn.; MAB2244), CD81 (Santa Cruz Biotechnology, Santa Cruz, Calif.; sc-7637), CD117 (Southern Biotech; 9816-01), CD133 (Abcam, Cambridge, Mass.; ab5558), CD184 (Becton-Dickinson, San Jose, Calif.; 555971), CD252 (R&D Systems; MAB10541) at the manufacturers' recommended concentrations or at 10 ug/ml, or an equivalent concentration of mouse isotype control IgG1, IgG2a or IgG2b (Southern Biotech). The cells were then stained with Alexa Fluor 488-conjugated goat anti-mouse IgG (H+L) antibody (Invitrogen, Carlsbad, Calif.; A11029) and analyzed using a FACSCalibur flow cytometer (Becton-Dickinson) and FloJo software (Tree Star, Inc. Ashland, Oreg.).

ELISA. Cell culture medium from selected cell lines were quantitated for factors secreted into the medium utilizing the following ELISA or Duoset (R & D Systems) kits: Amphiregulin (Catalog # DY262, R & D Systems, Minneapolis; MN), FGF-7/KGF (Catalog # DY251, R & D Systems, Minneapolis, Minn.), IGFBP-5 (Catalog # DY875, R & D Systems, Minneapolis, Minn.), PDGF-BB (Catalog # DY220, R & D Systems, Minneapolis, Minn.), TGFb-1 (Catalog # DY240, R & D Systems, Minneapolis, Minn.), TIMP-1 (Catalog # DY970, R & D Systems, Minneapolis, Minn.), Vitronectin (Catalog # TAK-MK102, Takara Bio distributed by Thermo Fisher Scientific, Waltham, Mass.). The factors were quantitated in duplicate determinations.

### Telomerase Assays and TRF Analysis

Telomeric Repeat Amplification Protocol (TRAP) assays were performed using a TRAPez Kit (Chemicon). CHAPS lysates were prepared from cells, and aliquots were frozen. Upon thawing, the lysates were subjected to protein quantification using the quick-start Bradford assay system (Biorad). Twenty six cycle PCR-TRAPs were performed in linear range of the assay using 300 ng of total protein lysate per reaction. TRAP products were resolved on 15% polyacrylamide large gels and exposed to phosphorimager screens. TRAP was performed as described above. Telomere length Restriction Fragment length (TRF) analysis was performed as described before48. In brief, genomic DNA was extracted from cells at different population doublings and subjected to restriction with Hinfl and RsaI and 2 μg of the digested DNA was resolved on 0.5% agarose gels. The resulting denatured gels were directly incubated with a telomeric 32P labeled (C3TA2)3 probe. The dried gels were subsequently washed and exposed to phoshoimager screens for detection of the telomeric signal.

See the Description of Figures above (Brief Description of the Drawings section) for FIGS. 32 to 42 and Supplementary Tables, which are from West et al., 2008, Regenerative Medicine vol. 3(3) pp. 287-308, incorporated by reference herein in its entirety.

### REFERENCES CITED IN EXAMPLE 51

1. Thomson, J. A. et al. Embryonic stein cell lines derived from human blastocysts. Science 282, 1145-1147 (1998).
2. Shamblott, M. J. et al. Derivation of pluripotent stem cells from cultured human primordial germ cells. Proc Natl Acad Sci USA 95, 13726-13731 (1998).
3. Blentic, A., Tandon, P., Payton, S., Walshe, J., Carney, T., Kelsh, R. N., Mason, I., & Graham, A. The emergence of ectomesenchyme. Dev. Dyn. January 25 [Epub ahead of print] (2008).
4. Carletti, B., Grimaldi, P., Magrassi, L., & Rossi, F. Specification of cerebellar progenitors after heterotopic-heterochronic transplantation to the embryonic CNS in vivo and in vitro. J. Neurosci. 22, 7132-7146 (2002).
5. Minguet, S. et al. A population of c-Kit(low)(CD45/TER119)-hepatic cell progenitors of 11-day postcoitus mouse embryo liver reconstitutes cell-depleted liver organoids. J. Clin. Invest. 112, 1152-1163.
6. Quackenbush, E. J., Wershil, B. K., Aguirre, V., & Gutierrez-Ramos, J. C. Eotaxin modulates myelopoiesis and mast cell development from embryonic hematopoietic progenitors. Blood 92, 1887-1897 (1998).
7. Kitisin, K. et al. Tgf-Beta signaling in development. Sci STKE 2007, cml (2007).
8. Correia, A. S., Anisimov, S. V., Li, J. Y. & Brundin, P. Stem cell-based therapy for Parkinson's disease. Ann Med 37, 487-498 (2005).
9. Abranches, E., Bekman, E., Henrique, D. & Cabral, J. M. Expansion of mouse embryonic stem cells on microcarriers. Biotechnol Bioeng 96, 1211-1221 (2007).
10. Smith, J. R., Pereira-Smith, O. M., & Schneider, E. L. Colony size distributions as a measure of in vivo and in vitro aging. PNAS(USA) 75, 1353-1356 (1978).
11. Rinn, J. L., Bondre, C., Gladstone, H. B., Brown, P. O., & Chang, H. Y. Anatomic demarcation by positional variation in fibroblast gene expression programs. PLoS Genet 2:e119 (2006).
12. McGinnis, W. & Krumlauf, R. Homeobox genes and axial patterning. Cell 68, 283-302 (1992).
13. Candia, A. F. et al. Mox-1 and Mox-2 define a novel homeobox gene subfamily and are differentially expressed during early mesodermal patterning in mouse embryos. Development 116, 1123-1136 (1992).
14. Candia, A. F. & Wright, C. V. Differential localization of Mox-1 and Mox-2 proteins indicates distinct roles during development. Int J Dev Biol 40, 1179-1184 (1996).
15. Aitola, M., Carlsson, P., Mahlapuu, M., Enerback, S. & Pelto-Huikko, M. Forkhead transcription factor FoxF2 is expressed in mesodermal tissues involved in epithelio-mesenchymal interactions. Dev Dyn 218, 136-149 (2000).
16. Sasaki, H. & Hogan, B. L. Differential expression of multiple fork head related genes during gastrulation and axial pattern formation in the mouse embryo. Development 118, 47-59 (1993).
17. Kim, T. A. et al. The BTB domain of the nuclear matrix protein NRP/B is required for neurite outgrowth. J Cell Sci 118, 5537-5548 (2005).
18. Kim, T. A. et al. NRP/B, a novel nuclear matrix protein, associates with p110(RB) and is involved in neuronal differentiation. J Cell Biol 141, 553-566 (1998).
19. Kitanaka, J., Takemura, M., Matsumoto, K., Mori, T. & Wanaka, A. Structure and chromosomal localization of a murine LIM/homeobox gene, Lhx8. Genomics 49, 307-309 (1998).
20. Al-Shawi, R., Ashton, S. V., Underwood, C. & Simons, J. P. Expression of the Ror1 and Ror2 receptor tyrosine kinase genes during mouse development. Dev Genes Evol 211, 161-171 (2001).
21. Blaschke, R. J., Monaghan, A. P., Schiller, S., Schechinger, B., Rao, E., Padilla-Nash, H., Ried, T., & Rappold, G. A. SHOT, a SHOX-related homeobox gene, is implicated in craniofacial, brain, heart, and limb development. PNAS(USA) 95, 2406-2411 (1998).
22. Stipp, C. S., Litwack, E. D. & Lander, A. D. Cerebroglycan: an integral membrane heparan sulfate proteoglycan that is unique to the developing nervous system and expressed specifically during neuronal differentiation. J Cell Biol 124, 149-160 (1994).
23. Ribes, V., Fraulob, V., Petkovieh, M. & Dolle, P. The oxidizing enzyme CYP26a1 tightly regulates the availability of retinoic acid in the gastrulating mouse embryo to ensure proper head development and vasculogenesis. Dev Dyn 236, 644-653 (2007).
24. Besser, D. Expression of nodal, lefty-a, and lefty-B in undifferentiated human embryonic stem cells requires activation of Smad2/3. J Biol Chem 279, 45076-45084 (2004).
25. Wang, Z. X. et al. Zfp206 is a transcription factor that controls pluripotency of embryonic stem cells. Stem Cells 25, 2173-2182 (2007).
26. Lim, L. S. et al. Zic3 is required for maintenance of pluripotency in embryonic stem cells. Mol Biol Cell 18, 1348-1358 (2007).
27. Thuret, S., Bhatt, L., O'Leary, D. D. & Simon, H. H. Identification and developmental analysis of genes expressed by dopaminergic neurons of the substantia nigra pars compacta. Mol Cell Neurosci 25, 394-405 (2004).
28. Roubin, R. et al. Structure and developmental expression of mouse Garp, a gene encoding a new leucine-rich repeat-containing protein. Int J Dev Biol 40, 545-555 (1996).
29. Hidai, C. et al. Cloning and characterization of developmental endothelial locus-1: an embryonic endothelial cell protein that binds the alphavbeta3 integrin receptor. Genes Dev 12, 21-33 (1998).
30. Koch, M. et al. Collagen XXIV, a vertebrate fibrillar collagen with structural features of invertebrate collagens: selective expression in developing cornea and bone. J Biol Chem 278, 43236-43244 (2003).
31. Goldberg, J. L. et al. An oligodendrocyte lineage-specific semaphorin, Sema5A, inhibits axon growth by retinal ganglion cells. J Neurosci 24, 4989-4999 (2004).
32. Rinn, J. L., Bondre, C., Gladstone, H. B., Brown, P. O. & Chang, H. Y. Anatomic demarcation by positional variation in fibroblast gene expression programs. PLoS Genet 2, e119 (2006).
33. Dahlstrand, J., Lardelli, M. & Lendahl, U. Nestin mRNA expression correlates with the central nervous system progenitor cell state in many, but not all, regions of developing central nervous system. Brain Res Dev Brain Res 84, 109-129 (1995).
34. Falk, J., Bonnon, C., Girault, J. A. & Faivre-Sarrailh, C. F3/contactin, a neuronal cell adhesion molecule implicated in axogenesis and myelination. Biol Cell 94, 327-334 (2002).
35. Besnard, V., Wert, S. E., Kaestner, K. H., Whitsett, J. A. Stage-specific regulation of Foxa1 and Foxa2 in mouse embryos and adult tissues. Gene Expr Patterns 5, 193-208 (2004).
36. Cereghini, S., Ott, M. O., Power, S., & Maury, M. Expression patterns of nHNF1 and HNF1 homeoproteins in early postimplantation embryos suggest distinct and sequential developmental roles. Development 116, 783-797 (1992).
37. Zhou, J., Wang, H., Lu, A., Hu, G., Luo, A., Ding, F., Zhang, J., Wang, X., Wu, M., & Liu, Z. A novel gene, NMES1, downregulated in human esophageal squamous cell carcinoma. Int J Cancer 101, 311-316 (2002).
38. Moll, R., Simbelmann, R., Goldschmidt, M. D., Keith, M., Laufer, J., Kasper, M., Koch, P. J., & Franke, W. W. The human gene encoding cytokeratin 20 and its expression during fetal development and in gastrointestinal carcinomas. Differentiation 53, 75-93 (1993).
39. Karsch-Mizrachi, I., Travis, M., Blau, H. & Leinwand, L. A. Expression and DNA sequence analysis of a human embryonic skeletal muscle myosin heavy chain gene. Nucleic Acids Res 17, 6167-6179 (1989).
40. Kachinsky, A. M., Dominov, J. A. & Miller, J. B. Intermediate filaments in cardiac myogenesis: nestin in the developing mouse heart. J Histochem Cytochem 43, 843-847 (1995).
41. Wagner, S. N., Wagner, C., Hofler, H., Atkinson, M. J. & Goos, M. Expression cloning of the cDNA encoding a melanoma-associated Ag recognized by mAb HMB-45. Identification as melanocyte-specific Pmel 17 cDNA. Lab Invest 73, 229-235 (1995).
42. Baxter, L. L. & Pavan, W. J. Pmel17 expression is Mitf-dependent and reveals cranial melanoblast migration during murine development. Gene Expr Patterns 3, 703-707 (2003).
43. Allsopp, R. C., Vaziri, H. Patterson, C., Goldstein, S., Younglai, E. V., Futcher, A. B., Greider, C. W., and Harley, C. B. Telomere length predicts replicative capacity of human fibroblasts. PNAS(USA), 89, 10114-10118 (1992).
44. West, M. D. & Mason, C. Mapping the human embryome: 1 to 10e13 and all the cells in between. Regen Med 2, 329-333 (2007).
45. Lee, D. D. & Seung, H. S. Learning the parts of objects by non-negative matrix factorization. Nature 401, 788-791 (1999).
46. Brunet, J. P., Tamayo, P., Golub, T. R. & Mesirov, J. P. Metagenes and molecular pattern discovery using matrix factorization. Proc Natl Acad Sci USA 101, 4164-4169 (2004).
47. Reich, M. et al. GenePattern 2.0. Nat Genet. 38, 500-501 (2006).
48. Vaziri, H. et al. ATM-dependent telomere loss in aging human diploid fibroblasts and DNA damage lead to the post-translational activation of p53 protein involving poly(ADP-ribose) polymerase. Embo J 16, 6018-6033 (1997).

### Example 52

The following example provides methods for producing terminally differentiated cells from relatively undifferentiated cells described herein. These representative differentiation protocols work on embryonic progenitor cell lines of the present invention, where the embryonic progenitor cell lines are mesodermal or neural crest-derived undifferentiated mesenchyme.

hES-cell derived neural crest cells are first cultured in αMEM containing 10% Fetal Bovine Serum for 42 weeks in uncoated tissue-culture grade dishes. FACS sorting of the cells is performed, after which the cells are placed in the following four different conditions for generation of adipocytes, chondrocytes, osteocytes and myocytes, respectively.

1) For the generation of adipocytes, the mesenchymal precursor cells are grown to confluence and exposed to 1 mM dexamethasone, 10 mg/ml insulin, and 0.4 mM isobutylxanthine in αMEM medium with 10% FBS for 2-4 weeks.

2) For the generation of chondrocytes, the mesenchymal precursor cells are exposed 10 ng/ml TGFb-3 and 200 mMAA in αMEM medium with 10% FBS for 3-4 weeks.

3) For the generation of osteocytes, the mesenchymal precursor cells are plated with 10 mM □-glycerol phosphate, 0.1 mM dexamtethasone, and 200 mM AA in αMEM medium with 10% FBS for 3-4 weeks.

4) For the generation of myocytes, FACS sorting for NCAM expression is performed on mesenchymal precursor cells that have been passaged in αMEM medium with 10% FBS. The NCAM+ cells are grown to confluence in the αMEM medium with 10% FBS and induced to differentiate with N2 medium. For differentiation of neural crest cells into peripheral nerve cells, the hES cell derived NCS cells that are FGF2/EGF expanded are placed in medium that contains BDNF, GDNF, NGF, and dbcAMP. For differentiation of neural crest cells into Schwann cells, the hES cell derived NCS cells that are FGF2/EGF expanded are placed in medium that contains CNTF, neuregulin, bFGF (10 ng/ml) and dbcAMP in addition to BDNF, GDNF and NGF.

### REFERENCES

Lee, G., H. Kim, et al. (2007). “Isolation and directed differentiation of neural crest stem cells derived from human embryonic stem cells.” Nat Biotechnol 25(12): 1468-75.
Barberi, T., L. Willis, et al. (2005). “Derivation of Multipotent Mesenchymal Precursors from Human Embryonic Stem Cells.” PLOS Medicine 2(6): 554-560.

### Example 53

The cells of the present invention are useful for the discovery of ligands such as antibodies and phage displayed and selected ligands that differentially bind to specific early embryonic cell types. By way of example, the cell lines of the present invention B16b, J13, J16, SK17, and B2 were exposed to a 12mer peptide phage display library. Sequencing of the phage revealed enrichment of sequences that were specific to particular cell lines and others that were common to all of the lines.

### Example 54

Tables 14 to 32 provide gene expression data for specific cell types (using Illumina and Affymetrix platforms as indicated). The genes listed are rank ordered, with genes at the top of each column are preferred.

The number shown in the tables is the fold-over or fold-under the mean value of that gene's expression in all the lines tested. In using these tables, one skilled in the art could choose a cell line(s) that expresses a particular secreted protein of interest to them, in certain cases selecting a cell in which the gene of interest is expressed at the highest value over the mean. As another example, in the case of surface-expressed antigens, one would choose screen for the expression of antigens having relatively high or low expression levels that would aid in the separation of the cell type of interest (e.g., by FACS).

The data provided in these tables can be used for any variety of purposes, which are apparent to those in the art, and as such any use of the data described herein is not meant to be limiting.

### Example 55

An example of a functional differentiation assay utilizing the cells of the present invention uses micromass and pellet protocols well known in the art as capable of causing bone marrow, adipose, and tooth-derived mesenchymal stem cells to differentiate into chondrogenic lineages. To demonstrate that individual cell lines are capable of differentiating into chondrogenic lineages we assayed by qPCR transcript levels for COL2A1, ACAN, CRTL1, CILP, BGN, and CEP68. In the case of the Chondrogenic Pellet Protocol,

1. Cells are cultured in gelatin (0.1%) coated Corning tissue culture treated cultureware and detached with 0.25% trypsin/EDTA (Invitrogen, Carlsbad, Calif., Gibco) diluted 1:3 with PBS (Ca, Mg free). After detachment and addition of growth medium cells are counted using a Coulter counter and appropriate number of cells needed for experiment (e.g. 10×10e6 or more) are transferred into a sterile polyproylene tube and spun at 150 g for 5 min at room temperature.

2. The supernatant is aspirated and discarded. The cells are washed with the addition of Incomplete Chondrogenic Medium consisting of hMSC Chondro BulletKit (PT-3925) to which is added supplements (Lonza, Basel, Switzerland, Poietics Single-Quots, Cat. # PT-4121). Supplements added to prepare Incomplete Chondrogenic Medium are: Dexamethasone (PT-4130G), Ascorbate (PT-4131G), ITS+supplements (4113G), Pyruvate (4114G), Proline (4115G), Gentamicin (4505G), Glutamine (PT-4140G).

3. Cells are spun at 150 g at room temperature, the supernatant is aspirated and cell the pellet is resuspended (once more) with 1.0 ml Incomplete Chondrogenic Medium per 7.5×10⁵ cells, and spun at 150×g for 5 minutes. The supernatant is aspirated and discarded. The Chondrogenesis culture protocol as described by Lonza is followed with some modifications (as written below).

4. Cell pellets are resuspended in Complete Chondrogenic medium to a concentration of 5.0×10⁵ cells per ml. Complete Chondrogenic Medium consists of Lonza Incomplete Medium plus TGFb3 (Lonza, PT-4124). Sterile lyophilized TGFb3 is reconstituted with the addition of sterile 4 mM HCl containing 1 mg/ml BSA to a concentration of 20 ug/ml and is stored after aliquoting at −80° C. Complete Chondrogenic medium is prepared just before use by the addition of 1 ul of TGFb3 for each 2 ml of Incomplete Chondrogenic medium (final TGFb3 concentration is 10 ng/ml).

5. An aliquot of 0.5 ml (2.5×10⁵ cells) of the cell suspension is placed into sterile 15 ml polypropylene culture tubes. Cells are spun at 150×g for 5 minutes at room temperature.

6. Following centrifugation the caps of the tubes are loosened one half turn to allow gas exchange. The tubes are placed in an incubator at 37° C., in a humidified atmosphere of 10% CO2 and 5% O2. Pellets are not disturbed for 24 hours.

7. Cell pellets are fed every 2-3 days by completely replacing the medium in each tube by aspirating the old medium with sterile 1-200 ul pipette tip and adding 0.5 ml of freshly prepared Complete Chondrogenic Medium to each tube.

8. After replacing the medium and ensuring that the pellet is free-floating, caps are loosened and tubes returned to the incubator.

9. Pellets are harvested after varying time points in chondrogenic medium and prepared for histology by fixation with Neutral Buffered Formalin and/or the pellets are combined and prepared for RNA extraction using RNeasy mini Kits (Qiagen, Germantown, Md., Cat. No. 74104).

The protocol for RNA extraction is followed as described by the Qiagen Handbook. RNA yield is maximized by using Qiagen's QiaShredder (Cat. #79654) to homogenize samples following lysis of cell pellets with RLT buffer (provided in RNeasy mini kits) prior to RNA extraction.

In the case of chondrogenic differentiation protocols using 10 ul micromass culture instead of pellets:

1. Cells are cultured in gelatin (0.1%) coated Corning tissue culture treated cultureware and detached with 0.25% trypsin/EDTA (Gibco) diluted 1:3 with PBS (Gibco Ca, Mg free). After detachment and addition of growth medium cells are counted using a Coulter counter and appropriate number of cells needed for experiment (e.g. 10×10e6 cells or more) are resuspended at a cell density of 20×10e6 cells/ml in growth medium.

2. 10 ul aliquots are seeded onto Corning Tissue Culture Treated Polystyrene plates or dishes. Twenty five or more micromass aliquots (200,000 cells/10 ul aliquot) are seeded.

3. The seeded micromasses are placed in a humidified incubator at 37° with 5% O₂ and 10% CO₂ for 90 minutes to 2 hours for attachment.

4. Growth medium is added and the following morning is replaced, after aspiration and washing with PBS (Ca, Mg free), with Complete Chondrogenic Medium (prepared as described above for the pellet micromasses). For example 6 ml Complete Chondrogenic medium/10 cm dish is added. Cells are maintained in a humidified incubator at 37° with 5% O₂, 10% CO₂ and chondrogenic medium replaced with freshly prepared medium every 2-3 days.

5. After varying periods of time in chondrogenic medium RNA is extracted using Qiagen RNeasy kits (Qiagen Cat. No. 74104) as described in the Qiagen Handbook. RNA yield is maximized by using Qiagen's QiaShredder (Cat. #79654 to homogenize samples following lysis of micromasses with RLT buffer, (which is provided with the RNeasy mini kits) prior to RNA extraction

An alternative to Lonza Chondrogenic medium is CellGro (Cat. No. 15-013-CV). from Media Tech and add to each 500 ml the following supplements are added: 5.0 ml Pen/Strep (Gibco Cat. No. 15140), 5.0 ml Glutamax (Gibco Cat. No. 35050), Dexamethasone (Sigma, St. Louis, Mo., Cat. No. D1756-100) −500 ul of 0.1 mM for a final concentration of 0.1 uM; L-Proline (Sigma Cat. No. D49752) −500 ul 0.35M; Final concentration of 0.35 mM; Ascorbic Acid-2-phosphate (Sigma, Cat. No. 49792, Fluka) −500 ul 0.17M. Final concentration 0.17 mM; ITS Premix (BD, Franklin Lakes, N.J., sterile Cat. No. 47743-628) −500 ul of 1000× concentrate Final 6.25 ug/ml insulin, 6.25 ug/ml transferrin, 6.25 ng/ml selenious acid, serum albumin 1.25 mg/ml, 5.35 ug/ml linoleic acid.

Following addition of constituents above the media is filtered through a 500 ml Corning 0.2 micron filter unit.

As an alternative to Lonza TGFb3 described above we use TGFb3 (R&D Systems, Minneapolis Minn., Cat. No. 243-B3-010). It is prepared, aliquoted and stored and used similarly to that purchased from Lonza.

The cell lines of the present invention EN13, EN47, EN31, EN2, Z11, 7SMOO7, 7PEND24, and 4D20.8 were assayed as described above compared to bone marrow mesenchymal stem cells passage 3 (Lonza), and normal human articular chonodrocytes. After 14 days of micromass and pellet chondrogenic conditions as described, the lines Z11, 7PEND24, and 4D20.8 expressed elevated COL2A1 expression, with 4D20.8 expressing higher relative levels of transcript than normal human articular chondrocytes. Bone marrow mesenchymal stem cells at passage 3 expressed little if any transcript. The lines Z11, 7PEND24, and 4D20.8 express markers of neural crest and therefore are useful in modeling neural crest chondrogenesis and in clinical cell-based therapy, such as where said cell types are manufactured from hES, hED, or hiPS parental pluripotent stem cells, and transplanted for the repair of cartilage defects such as arthritis, for trauma such as in the induction of bone formation, mandibular atrophy, and related bone and cartilage degenerative disease. The cell line 4D20.8 strongly expresses the marker gene LHX8, a marker of perioral mesenchyme, such as that producing the secondary palate and would therefore be useful in the repair of cleft palate.

### Example 56

The cell lines 7PEND24, and 4D20.8 along with control bone marrow mesenchymal stem cells (Lonza) adult dental pulp mesenchymal stem cells, and foreskin dermal fibroblasts were synchronized in growth arrest with 0.5% serum containing media as described in Example 29, or differentiated in chondrogenic conditions as pellets or micromasses for 1, 2, or 14 days. RNA was harvested as described herein and hybridized to Illimina Human Ref-8 v3 microarrays for gene expression analysis. Bone marrow mesenchymal stem cells responded to both pellet and micromass chondrogenic conditions with a marked up-regulation of chondrocyte gene expression. Examples of chondrocyte differentiation markers include COL2A1, MGP, MATN4, PENK, EPYC, COL9A2, and LECT1. While COL2A1, EPYC, MATN4, and LECT1, induction are relatively specific to chondrogenesis, the genes PENK and MGP are more nonspecific. A comparison of gene expression in the undifferentiated vs 14 days in micromass conditions in the cell line D20.8 showed an upregulation of MGP expression of 479×, MATN4 of 10×, PENK of 369×, COL2A1 of 60×, EPYC of 42×, COL9A2 of 25×, LECT1 of 24×, and similarly, with MSCs, the differentiation showed an upregulation of MGP expression of 5× (though the undifferentiated MSCs expressed relatively high basal levels of expression unlike 4D20.8), MATN4 of 20×, PENK of 6× (again, relatively high levels in undifferentiated MSCs compared to no expression in undifferentiated 4D20.8), COL2A1 of 613×, EPYC of 48×, COL9A2 of 117×, LECT1 of 34×. In contract, dermal fibroblasts showed an upregulation of MGP expression of 37×, PENK of 369× (as expected since these are not strictly chondrocyte-specific), but no expression of COL2A1, EPYC, or COL9A2 either before or after experimental treatment (consistent with them making some, but not chondrocyte-specific markers). The wisdom tooth-derived dental pulp mesenchymal stem cells showed an induction of MGP expression of 74×, COL9A2 of 3×, PENK of 4×, and unlike mesenchymal stem cells and 4D20.8 no induction of COL2A1, EPYC, LECT1, or MATN4. Therefore, the cell line of the present invention 4D20.8, while showing site-specific homeobox gene expression of perioral mesenchyme, such as LHX8 similar to the dental pulp mesenchymal stem cells, they nevertheless were distinct from both the bone marrow mesenchymal stem cells in numerous markers. The bone marrow mesenchymal stem cells were positive for caudal HOX gene expression and PITX1 (a marker of lower limbs), but negative for LHX8, while the line 4D20.8 expressed no HOX genes, were LHX8+, but unlike dental pulp mesenchyme, 4D20.8 expressed numerous genes differently, including those of robust chondrogenesis, consistent with their role in normal development in forming the palate and mandible. The cell line 7PEND24 showed detectable though lower levels of chondrocyte markers.

TABLE I
Culture Variables

EGF Ligands

| | |
|---|---|
| 1) | Amphiregulin |
| 2) | Betacellulin |
| 3) | EGF |
| 4) | Epigen |
| 5) | Epiregulin |
| 6) | HB-EGF |
| 7) | Neuregulin-3 |
| 8) | NRG1 isoform GGF2 |
| 9) | NRG1 Isoform SMDF |
| 10) | NRG1-alpha/HRG1-alpha |
| 11) | TGF-alpha |
| 12) | TMEFF1/Tomoregulin-1 |
| 13) | TMEFF2 |
| 14) | EGF Ligands pooled (1-13 above) |

EGF R/ErbB Receptor Family

| | |
|---|---|
| 15) | EGF Receptor |
| 16) | ErbB2 |
| 17) | ErbB3 |
| 18) | ErbB4 |
| 19) | EGF/ErbB Receptors pooled (15-18 above) |

EGF Ligands

| | |
|---|---|
| 20) | FGF acidic |
| 21) | FGF basic |
| 22) | FGF-3 |
| 23) | FGF-4 |
| 24) | FGF-5 |
| 25) | FGF-6 |
| 26) | KGF/FGF-7 |
| 27) | FGF-8 |
| 28) | FGF-9 |
| 29) | FGF-10 |
| 30) | FGF-11 |
| 31) | FGF-12 |
| 32) | FGF-13 |
| 33) | FGF-14 |
| 34) | FGF-15 |
| 35) | FGF-16 |
| 36) | FGF-17 |
| 37) | FGF-18 |
| 38) | FGF-19 |
| 39) | FGF-20 |
| 40) | FGF-21 |
| 41) | FGF-22 |
| 42) | FGF-23 |
| 43) | FGF Ligands pooled (20-38 above) |

FGF Receptors

| | |
|---|---|
| 40) | FGF R1 |
| 41) | FGF R2 |
| 42) | FGF R3 |
| 43) | FGF R4 |
| 44) | FGF R5 |
| 45) | FGF Receptors pooled (40-44 above) |

FGF Regulators

| | |
|---|---|
| 46) | FGF-BP |

Hedgehogs

| | |
|---|---|
| 47) | Desert Hedgehog |
| 48) | Sonic Hedgehog |
| 49) | Indian Hedgehog |
| 50) | Hedgehogs pooled (47-49 above) |

Hedgehog Regulators

| | |
|---|---|
| 51) | Gas1 |
| 52) | Hip |
| 53) | Hedgehog Regulators pooled (51-52 above) |

IGF Ligands

| | |
|---|---|
| 54) | IGF-I |
| 55) | IGF-II |
| 56) | IGF Ligands pooled (54-55 above) |

IGF-I Receptor (CD221)

| | |
|---|---|
| 57) | IGF-1 R |

GF Binding Protein (IGFBP) Family

| | |
|---|---|
| 58) | ALS |
| 59) | IGFBP-4 |
| 60) | CTGF/CCN2 |
| 61) | IGFBP-5 |
| 62) | Endocan |
| 63) | IGFBP-6 |
| 64) | IGFBP-1 |
| 65) | IGFBP-rp1/IGFBP-7 |
| 66) | IGFBP-2 |
| 67) | NOV/CCN3 |
| 68) | IGFBP-3 |
| 69) | GF Binding Protein Family pooled (58-68 |
| | above) |

Receptor Tyrosine Kinases

| | |
|---|---|
| 70) | Ax1 |
| 71) | Clq R1/CD93 |
| 72) | DDR1 |
| 73) | Flt-3 |
| 74) | DDR2 |
| 75) | HGF R |
| 76) | Dtk |
| 77) | IGF-II R |
| 78) | Eph |
| 79) | Insulin R/CD220 |
| 80) | EphA1 |
| 81) | M-CSF R |
| 82) | EphA2 |
| 83) | Mer |
| 84) | EphA3 |
| 85) | MSP R/Ron |
| 86) | EphA4 |
| 87) | MuSK |
| 88) | EphA5 |
| 89) | PDGF R alpha |
| 90) | EphA6 |
| 91) | PDGF R beta |
| 92) | EphA7 |
| 93) | Ret |
| 94) | EphA8 |
| 95) | ROR1 |
| 96) | EphB1 |
| 97) | ROR2 |
| 98) | EphB2 |
| 99) | SCF R/c-kit |
| 100) | EphB3 |
| 101) | Tie-1 |
| 102) | EphB4 |
| 103) | Tie-2 |
| 104) | EphB6 |
| 105) | TrkA |
| 106) | TrkB |
| 107) | TrkC |
| 108) | VEGF R1/Flt-1 |
| 109) | VEGF R2/Flk-1 |
| 110) | VEGF R3/Flt-4 |
| 111) | Receptor Tyrosine Kinases pooled (70-110 |
| | above) |

Proteoglycans

| | |
|---|---|
| 112) | Aggrecan |
| 113) | Lumican |
| 114) | Biglycan |
| 115) | Mimecan |
| 116) | Decorin |
| 117) | NG2/MCSP |
| 118) | Endocan |
| 119) | Osteoadherin |
| 120) | Endorepellin |
| 121) | Syndecan-1/CD138 |
| 122) | Glypican 2 |
| 123) | Syndecan-3 |
| 124) | Glypican 3 |
| 125) | Testican 1/SPOCK1 |
| 126) | Glypican 5 |
| 127) | Testican 2/SPOCK2 |
| 128) | Glypican 6 |
| 129) | Testican 3/SPOCK3 |
| 130) | Heparan sulfate proteoglycan |
| 131) | Heparin |
| 132) | Chondroitin sulfate proteoglycan |
| 133) | Hyaluronic acid |
| 134) | Dermatan sulfate proteoglycan |

Proteoglycan Regulators

| | |
|---|---|
| 135) | Arylsulfatase A/ARSA |
| 136) | HAPLN1 |
| 137) | Exostosin-like 2 |
| 138) | HS6ST2 |
| 139) | Exostosin-like 3 |
| 140) | IDS |
| 141) | Proteoglycan Regulators pooled (135-140 |
| | above) |

SCF, Flt-3 Ligand & M-CSF

| | |
|---|---|
| 142) | Flt-3 |
| 143) | M-CSF R |
| 144) | Flt-3 Ligand |
| 145) | SCF |
| 146) | M-CSF |
| 147) | SCF R/c-kit |
| 148) | Pooled factors (142-147 above) |

Activins

| | |
|---|---|
| 149) | Activin A |
| 150) | Activin B |
| 151) | Activin AB |
| 152) | Activin C |
| 153) | Pooled Activins (149-152 above) |

BMPs (Bone Morphogenetic Proteins)

| | |
|---|---|
| 154) | BMP-2 |
| 155) | BMP-3 |
| 156) | BMP-3b/GDF-10 |
| 157) | BMP-4 |
| 158) | BMP-5 |
| 159) | BMP-6 |
| 160) | BMP-7 |
| 161) | BMP-8 |
| 162) | Decapentaplegic |
| 163) | Pooled BMPs (154-162 above) |

GDFs (Growth Differentiation Factors)

| | |
|---|---|
| 164) | GDF-1 |
| 165) | GDF-2 |
| 166) | GDF-3 |
| 167) | GDF-4 |
| 168) | GDF-5 |
| 169) | GDF-6 |
| 170) | GDF-7 |
| 171) | GDF-8 |
| 172) | GDF-9 |
| 173) | GDF-10 |
| 174) | GDF-11 |
| 175) | GDF-12 |
| 176) | GDF-13 |
| 177) | GDF-14 |
| 178) | GDF-15 |
| 179) | GDFs pooled (164-178 above) |

GDNF Family Ligands

| | |
|---|---|
| 180) | Artemin |
| 181) | Neurturin |
| 182) | GDNF |
| 183) | Persephin |
| 184) | GDNF Ligands pooled (180-183 above) |

TGF-beta

| | |
|---|---|
| 185) | TGF-beta |
| 186) | TGF-beta 1 |
| 187) | TGF-beta 1.2 |
| 188) | TGF-beta 2 |
| 189) | TGF-beta 3 |
| 190) | TGF-beta 4 |
| 191) | TGF-beta 5 |
| 192) | LAP (TGF-beta 1) |
| 193) | Latent TGF-beta 1 |
| 194) | TGF-beta pooled (185-193 above) |

Other TGF-beta Superfamily Ligands

| | |
|---|---|
| 195) | Lefty |
| 196) | Nodal |
| 197) | MIS/AMH |
| 198) | Other TGF-beta Ligands pooled (195-197 |
| | above) |

TGF-beta Superfamily Receptors

| | |
|---|---|
| 199) | Activin RIA/ALK-2 |
| 200) | GFR alpha-1 |
| 201) | Activin RIB/ALK-4 |
| 202) | GFR alpha-2 |
| 203) | Activin RIIA |
| 204) | GFR alpha-3 |
| 205) | Activin RIIB |
| 206) | GFR alpha-4 |
| 207) | ALK-1 |
| 208) | MIS RII |
| 209) | ALK-7 |
| 210) | Ret |
| 211) | BMPR-IA/ALK-3 |
| 212) | TGF-beta RI/ALK-5 |
| 213) | BMPR-IB/ALK-6 |
| 214) | TGF-beta RII |
| 215) | BMPR-II |
| 216) | TGF-beta RIIb |
| 217) | Endoglin/CD105 |
| 218) | TGF-beta RIII |
| 219) | TGF-beta family receptors pooled (199-218 |
| | above) |

TGF-beta Superfamily Modulators

| | |
|---|---|
| 220) | Amnionless |
| 221) | GASP-2/WFIKKN |
| 222) | BAMBI/NMA |
| 223) | Gremlin |
| 224) | Caronte |
| 225) | NCAM-1/CD56 |
| 226) | Cerberus 1 |
| 227) | Noggin |
| 228) | Chordin |
| 229) | PRDC |
| 230) | Chordin-Like 1 |
| 231) | Chordin-Like 2 |
| 232) | Smad1 |
| 233) | Smad4 |
| 234) | Smad5 |
| 235) | Smad7 |
| 236) | Smad8 |
| 237) | CRIM1 |
| 238) | Cripto |
| 239) | Crossveinless-2 |
| 240) | Cryptic |
| 241) | SOST |
| 242) | DAN |
| 243) | Latent TGF-beta bp1 |
| 244) | TMEFF1/Tomoregulin-1 |
| 245) | FLRG |
| 246) | TMEFF2 |
| 247) | Follistatin |
| 248) | TSG |
| 249) | Follistatin-like 1 |
| 250) | Vasorin |
| 251) | GASP-1/WFIKKNRP |
| 252) | TGF Modulators pooled (220-251 above) |

VEGF/PDGF Family

| | |
|---|---|
| 253) | Neuropilin-1 |
| 254) | PIGF |
| 255) | PIGF-2 |
| 256) | Neuropilin-2 |
| 257) | PDGF |
| 258) | VEGF R1/Flt-1 |
| 259) | PDGF R alpha |
| 260) | VEGF R2/Flk-1 |
| 261) | PDGF R beta |
| 262) | VEGF R3/Flt-4 |
| 263) | PDGF-A |
| 264) | VEGF |
| 265) | PDGF-B |
| 266) | VEGF-B |
| 267) | PDGF-C |
| 268) | VEGF-C |
| 269) | PDGF-D |
| 270) | VEGF-D |
| 271) | PDGF-AB |
| 272) | VEGF/PDGF Family pooled (253-271 above) |

Dickkopf Proteins & Wnt Inhibitors

| | |
|---|---|
| 273) | Dkk-1 |
| 274) | Dkk-2 |
| 275) | Dkk-3 |
| 276) | Dkk-4 |
| 277) | Soggy-1 |
| 278) | WIF-1 |
| 279) | Pooled factors (273-278 above) |

Frizzled & Related Proteins

| | |
|---|---|
| 280) | Frizzled-1 |
| 281) | Frizzled-2 |
| 282) | Frizzled-3 |
| 283) | Frizzled-4 |
| 284) | Frizzled-5 |
| 285) | Frizzled-6 |
| 286) | Frizzled-7 |
| 287) | Frizzled-8 |
| 288) | Frizzled-9 |
| 289) | sFRP-1 |
| 290) | sFRP-2 |
| 291) | sFRP-3 |
| 292) | sFRP-4 |
| 293) | MFRP |
| 294) | Factors pooled (280-293 above) |

Wnt Ligands

| | |
|---|---|
| 295) | Wnt-1 |
| 296) | Wnt-2 |
| 297) | Wnt-3 |
| 298) | Wnt-3a |
| 299) | Wnt-4 |
| 300) | Wnt-5 |
| 301) | Wnt-5a |
| 302) | Wnt-6 |
| 303) | Wnt-7 |
| 304) | Wnt-8 |
| 305) | Wnt-8a |
| 306) | Wnt-9 |
| 307) | Wnt-10a |
| 308) | Win-10b |
| 309) | Wnt-11 |
| 310) | Win Ligands pooled (295-309 above) |

Other Wnt-related Molecules

| | |
|---|---|
| 311) | beta-Catenin |
| 312) | LRP-6 |
| 313) | GSK-3 |
| 314) | ROR1 |
| 315) | Kremen-1 |
| 316) | ROR2 |
| 317) | Kremen-2 |
| 318) | WISP-1/CCN4 |
| 319) | LRP-1 |
| 320) | Pooled factors (311-319 above) |

Other Growth Factors

| | |
|---|---|
| 321) | CTGF/CCN2 |
| 322) | NOV/CCN3 |
| 323) | EG-VEGF/PK1 |
| 324) | Osteocrin |
| 325) | Hepassocin |
| 326) | PD-ECGF |
| 327) | HGF |
| 328) | Progranulin |
| 329) | beta-NGF |
| 330) | Thrombopoietin |
| 331) | Pooled factors (321-330 above) |

Steroid Hormones

| | |
|---|---|
| 332) | 17beta-Estradiol |
| 333) | Testosterone |
| 334) | Cortisone |
| 335) | Dexamethasone |

Extracellular/Membrane Proteins

| | |
|---|---|
| 336) | Plasma Fibronectin |
| 337) | Tissue Fibronectin |
| 338) | Fibronectin fragments |
| 339) | Collagen Type I (gelatin) |
| 340) | Collagen Type II |
| 341) | Collagen Type III |
| 342) | Tenascin |
| 343) | Matrix Metalloproteinase 1 |
| 344) | Matrix Metalloproteinase 2 |
| 345) | Matrix Metalloproteinase 3 |
| 346) | Matrix Metalloproteinase 4 |
| 347) | Matrix Metalloproteinase 5 |
| 348) | Matrix Metalloproteinase 6 |
| 349) | Matrix Metalloproteinase 7 |
| 350) | Matrix Metalloproteinase 8 |
| 351) | Matrix Metalloproteinase 9 |
| 352) | Matrix Metalloproteinase 10 |
| 353) | Matrix Metalloproteinase 11 |
| 354) | Matrix Metalloproteinase 12 |
| 355) | Matrix Metalloproteinase 13 |
| 356) | ADAM-1 |
| 357) | ADAM-2 |
| 358) | ADAM-3 |
| 359) | ADAM-4 |
| 360) | ADAM-5 |
| 361) | ADAM-6 |
| 362) | ADAM-7 |
| 363) | ADAM-8 |
| 364) | ADAM-9 |
| 365) | ADAM-10 |
| 366) | ADAM-11 |
| 367) | ADAM-12 |
| 368) | ADAM-13 |
| 369) | ADAM-14 |
| 370) | ADAM-15 |
| 371) | ADAM-16 |
| 372) | ADAM-17 |
| 373) | ADAM-18 |
| 374) | ADAM-19 |
| 375) | ADAM-20 |
| 376) | ADAM-21 |
| 377) | ADAM-22 |
| 378) | ADAM-23 |
| 379) | ADAM-24 |
| 380) | ADAM-25 |
| 381) | ADAM-26 |
| 382) | ADAM-27 |
| 383) | ADAM-28 |
| 384) | ADAM-29 |
| 385) | ADAM-30 |
| 386) | ADAM-31 |
| 387) | ADAM-32 |
| 388) | ADAM-33 |
| 389) | ADAMTS-1 |
| 390) | ADAMTS-2 |
| 391) | ADAMTS-3 |
| 392) | ADAMTS-4 |
| 393) | ADAMTS-5 |
| 394) | ADAMTS-6 |
| 395) | ADAMTS-7 |
| 396) | ADAMTS-8 |
| 397) | ADAMTS-9 |
| 398) | ADAMTS-10 |
| 399) | ADAMTS-11 |
| 400) | ADAMTS-12 |
| 401) | ADAMTS-13 |
| 402) | ADAMTS-14 |
| 403) | ADAMTS-15 |
| 404) | ADAMTS-16 |
| 405) | ADAMTS-17 |
| 406) | ADAMTS-18 |
| 407) | ADAMTS-19 |
| 408) | ADAMTS-20 |
| 409) | Arg-Gly-Asp |
| 410) | Arg-Gly-Asp-Ser |
| 411) | Arg-Gly-Asp-Ser-Pro-Ala-Ser-Ser-Lys-Pro |
| 412) | Arg-Gly-Glu-Ser |
| 413) | Arg-Phe-Asp-Ser |
| 414) | SPARC |
| 415) | Cys-Asp-Pro-Gly-Tyr-Ile-Gly-Ser-Arg |
| 416) | Cys-Ser-Arg-Ala-Arg-Lys-Gln-Ala-Ala-Ser-Ile- |
| | Lys-Val-Ser-Ala-Asp-Arg |
| 417) | Elastin |
| 418) | Tropelastin |
| 419) | Gly-Arg-Gly-Asp-Ser-Pro-Lys |
| 420) | Gly-Arg-Gly-Asp-Thr-Pro |
| 421) | Laminin |
| 422) | Leu-Gly-Thr-Ile-Pro-Gly |
| 423) | Ser-Asp-Gly-Arg-Gly |
| 424) | Vitronectin |
| 425) | Superfibronectin |
| 426) | Thrombospondin |
| 427) | TIMP-1 |
| 428) | TIMP-2 |
| 429) | TIMP-3 |
| 430) | TIMP-4 |
| 431) | Fibromodulin |
| 432) | Flavoridin |
| 433) | Collagen IV |
| 434) | Collagen V |
| 435) | Collagen VI |
| 436) | Collagen VII |
| 437) | Collagen VIII |
| 438) | Collagen IX |
| 439) | Collagen X |
| 440) | Collagen XI |
| 441) | Collagen XII |
| 442) | Entactin |
| 443) | Fibrillin |
| 444) | Syndecan-1 |
| 445) | Keratan sulfate proteoglycan |

Ambient Oxygen

| | |
|---|---|
| 446) | 0.1-0.5% Oxygen |
| 447) | 0.5-1% Oxygen |
| 448) | 1-2% Oxygen |
| 449) | 2-5% Oxygen |
| 450) | 5-10% Oxygen |
| 451) | 10-20% Oxygen |

Animal Serum

| | |
|---|---|
| 452) | 0.1% Bovine Serum |
| 453) | 0.5% Bovine Serum |
| 454) | 1.0% Bovine Serum |
| 455) | 5.0% Bovine Serum |
| 456) | 10% Bovine Serum |
| 457) | 20% Bovine Serum |
| 458) | 10% Horse Serum |

Interleukins

| | |
|---|---|
| 459) | IL-1 |
| 460) | IL-2 |
| 461) | IL-3 |
| 462) | IL-4 |
| 463) | IL-5 |
| 464) | IL-6 |
| 465) | IL-7 |
| 466) | IL-8 |
| 467) | IL-9 |
| 468) | IL-10 |
| 469) | IL-11 |
| 470) | IL-12 |
| 471) | IL-13 |
| 472) | IL-14 |
| 473) | IL-15 |
| 474) | IL-16 |
| 475) | IL-17 |
| 476) | IL-18 |

Proteases

| | |
|---|---|
| 477) | MMP-1 |
| 478) | MMP-2 |
| 479) | MMP-3 |
| 480) | MMP-4 |
| 481) | MMP-5 |
| 482) | MMP-6 |
| 483) | MMP-7 |
| 484) | MMP-8 |
| 485) | MMP-9 |
| 486) | MMP-10 |
| 487) | MMP-11 |
| 488) | MMP-12 |
| 489) | MMP-13 |
| 490) | MMP-14 |
| 491) | MMP-15 |
| 492) | MMP-16 |
| 493) | MMP-17 |
| 494) | MMP-18 |
| 495) | MMP-19 |
| 496) | MMP-20 |
| 497) | MMP-21 |
| 498) | MMP-22 |
| 499) | MMP-23 |
| 500) | MMP-24 |
| 501) | Cathepsin B |
| 501) | Cathepsin C |
| 503) | Cathepsin D |
| 504) | Cathepsin G |
| 505) | Cathepsin H |
| 506) | Cathepsin L |
| 507) | Trypsin |
| 508) | Pepsin |
| 509) | Elastase |
| 510) | Carboxypeptidase A |
| 511) | Carboxypeptidase B |
| 512) | Carboxypeptidase G |
| 513) | Carboxypeptidase P |
| 514) | Carboxypeptidase W |
| 515) | Carboxypeptidase Y |
| 516) | Chymotrypsin |
| 517) | Plasminogen |
| 518) | Plasmin |
| 519) | u-type Plasminogen activator |
| 520) | t-type Plasminogen activator |
| 521) | Plasminogen activator inhibitor-1 |
| 522) | Carboxypeptidase Z |

Amino Acids

| | |
|---|---|
| 522) | Alanine |
| 523) | Arginine |
| 524) | Asparagine |
| 525) | Aspartic acid |
| 526) | Cysteine |
| 527) | Glutamine |
| 528) | Glutamic acid |
| 529) | Glycine |
| 530) | Histidine |
| 531) | Isoleucine |
| 532) | Leucine |
| 533) | Lysine |
| 534) | Methionine |
| 535) | Phenylalanine |
| 536) | Proline |
| 537) | Serine |
| 538) | Threonine |
| 539) | Tryptophan |
| 540) | Tyrosine |
| 541) | Valine |

Prostaglandins

| | |
|---|---|
| 542) | Prostaglandin A1 |
| 543) | Prostaglandin A2 |
| 544) | Prostaglandin B1 |
| 545) | Prostaglandin B2 |
| 546) | Prostaglandin D2 |
| 547) | Prostaglandin E1 |
| 548) | Prostaglandin E2 |
| 549) | Prostaglandin F1alpha |
| 550) | Prostaglandin F2alpha |
| 551) | Prostaglandin H |
| 552) | Prostaglandin I2 |
| 553) | Prostaglandin J2 |
| 554) | 6-Keto-Prostaglandin F1a |
| 555) | 16,16-Dimethyl-Prostaglandin E2 |
| 556) | 15d-Prostaglandin J2 |
| 557) | Prostaglandins pooled (542-556 above) |

Retinoid receptor agonists/Antagonists

| | |
|---|---|
| 558) | Methoprene Acid |
| 559) | All trans retinoic acid |
| 560) | 9-Cis Retinoic Acid |
| 561) | 13-Cis Retinoic Acid |
| 562) | Retinoid agonists pooled (558-561 above) |
| 563) | Retinoid antagonists |
| 564) | Retinoic acid receptor isotype RARalpha |
| 565) | Retinoic acid receptor isotype RARbeta |
| 566) | Retinoic acid receptor isotype RARgamma |
| 567) | Retinoic X receptor isotype RXRalpha |
| 568) | Retinoic X receptor isotype RXRbeta |
| 569) | Retinoic X receptor isotype RARgamma |

Miscellaneous Inducers

| | |
|---|---|
| 570) | Plant lectins |
| 571) | Bacterial lectins |
| 572) | forskolin |
| 573) | Phorbol myristate acetate |
| 574) | Poly-D-lysine |
| 575) | 1,25-dihydroxyvitamin D |
| 576) | Inhibin |
| 577) | Heregulin |
| 578) | Glycogen |
| 579) | Progesterone |
| 580) | IL-1 |
| 581) | Serotonin |
| 582) | Fibronectin-45 kDa Fragment |
| 583) | Fibronectin-70 kDa Fragment |
| 584) | glucose |
| 585) | beta mercaptoethanol |
| 586) | heparinase |
| 587) | pituitary extract |
| 588) | chorionic gonadotropin |
| 589) | adrenocorticotropic hormone |
| 590) | thyroxin |
| 591) | Bombesin |
| 592) | Neuromedin B |
| 593) | Gastrin-Releasing Peptide |
| 594) | Epinephrine |
| 595) | Isoproterenol |
| 596) | Ethanol |
| 597) | DHEA |
| 598) | Nicotinic Acid |
| 599) | NADH |
| 600) | Oxytocin |
| 601) | Vasopressin |
| 602) | Vasotocin |
| 603) | Angiotensin I |
| 604) | Angiotensin II |
| 605) | Angiotensin I Converting Enzyme |
| 606) | Angiotensin I Converting Enzyme Inhibitor |
| 607) | Chondroitinase AB |
| 608) | Chondroitinase C |
| 609) | Brain natriuretic peptide |
| 610) | Calcitonin |
| 611) | Calcium ionophore I |
| 612) | Calcium ionophore II |
| 613) | Calcium ionophore III |
| 614) | Calcium ionophore IV |
| 615) | Bradykinin |
| 616) | Albumin |
| 617) | Plasmonate |
| 618) | LIF |
| 619) | PARP inhibitors |
| 620) | Lysophosphatidic acid |
| 621) | (R)-METHANANDAMIDE |
| 622) | 1,25-DIHYDROXYVITAMIN D3 |
| 623) | 1,2-DIDECANOYL-GLYCEROL (10:0) |
| 624) | 1,2-DIOCTANOYL-SN-GLYCEROL |
| 625) | 1,2-DIOLEOYL-GLYCEROL (18:1) |
| 626) | 10-hydroxycamptothecin |
| 627) | 11,12-EPOXYEICOSATRIENOIC ACID |
| 628) | 12(R)-HETE |
| 629) | 12(S)-HETE |
| 630) | 12(S)-HPETE |
| 631) | 12-METHOXYDODECANOIC ACID |
| 632) | 13(S)-HODE |
| 633) | 13(S)-HPODE |
| 634) | 13,14-DIHYDRO-PGE1 |
| 635) | 13-KETOOCTADECADIENOIC ACID |
| 636) | 14,15-EPOXYEICOSATRIENOIC ACID |
| 637) | 1400 W |
| 638) | 15(S)-HETE |
| 639) | 15(S)-HPETE |
| 640) | 15-KETOEICOSATETRAENOIC ACID |
| 641) | 17-Allylamino-geldanamycin |
| 642) | 17-OCTADECYNOIC ACID |
| 643) | 17-PHENYL-TRINOR-PGE2 |
| 644) | 1-ACYL-PAF |
| 645) | 1-HEXADECYL-2-ARACHIDONOYL-522) |
| 646) | GLYCEROL |
| 647) | 1-HEXADECYL-2-METHYLGLYCERO-3 PC |
| 648) | 1-HEXADECYL-2-O-ACETYL-GLYCEROL |
| 649) | 1-HEXADECYL-2-O-METHYL-GLYCEROL |
| 650) | 1-OCTADECYL-2-METHYLGLYCERO-3 PC |
| 651) | 1-OLEOYL-2-ACETYL-GLYCEROL |
| 652) | 1-STEAROYL-2-LINOLEOYL-GLYCEROL |
| 653) | 1-STEAROYL-2-ARACHIDONOYL-GLYCEROL |
| 654) | 2,5-ditertbutylhydroquinone |
| 655) | 24(S)-hydroxycholesterol |
| 656) | 24,25-DIHYDROXYVITAMIN D3 |
| 657) | 25-HYDROXYVITAMIN D3 |
| 658) | 2-ARACHIDONOYLGLYCEROL |
| 659) | 2-FLUOROPALMITIC ACID |
| 660) | 2-HYDROXYMYRISTIC ACID |
| 661) | 2-methoxyantimycin A3 |
| 662) | 3,4-dichloroisocoumarin |
| 663) | granzyme B inhibitor |
| 664) | 4-AMINOPYRIDINE |
| 665) | 4-HYDROXYPHENYLRETINAMIDE |
| 666) | 4-OXATETRADECANOIC ACID |
| 667) | 5(S)-HETE |
| 668) | 5(S)-HPETE |
| 669) | 5,6-EPOXYEICOSATRIENOIC ACID |
| 670) | 5,8,11,14-EICOSATETRAYNOIC ACID |
| 671) | 5,8,11-EICOSATRITYNOIC ACID |
| 672) | 5-HYDROXYDECANOATE |
| 673) | 5-iodotubercidin |
| 674) | 5-KETOEICOSATETRAENOIC ACID |
| 675) | 5′-N-Ethylcarboxamidoadenosine (NECA) |
| 676) | 6,7-ADTN HBr |
| 677) | 6-FORMYLINDOLO [3,2-B] CARBAZOLE |
| 678) | 7,7-DIMETHYLEICOSADIENOIC ACID |
| 679) | 8,9-EPOXYEICOSATRIENOIC ACID |
| 680) | 8-methoxymethyl-IBMX |
| 681) | 9(S)-HODE |
| 682) | 9(S)-HPODE |
| 683) | 9,10-OCTADECENOAMIDE |
| 684) | A-3 |
| 685) | AA-861 |
| 686) | acetyl (N)-s-farnesyl-1-cysteine |
| 687) | ACETYL-FARNESYL-CYSTEINE |
| 688) | Ac-Leu-Leu-Nle-CHO |
| 689) | ACONITINE |
| 690) | actinomycin D |
| 691) | ADREINIC ACID (22:4, n-6) |
| 692) | 1 mM |
| 693) | AG-1296 |
| 694) | AG1478 |
| 695) | AG213 (Tyrphostin 47) |
| 696) | AG-370 |
| 697) | AG-490 |
| 698) | AG-879 |
| 699) | AGC |
| 700) | AGGC |
| 701) | Ala-Ala-Phe-CMK |
| 702) | alamethicin |
| 703) | Alrestatin |
| 704) | AM 92016 |
| 704) | AM-251 |
| 706) | AM-580 |
| 707) | AMANTIDINE |
| 708) | AMILORIDE |
| 709) | Amino-1,8-naphthalimide [4-Amino-1,8-522) |
| | naphthalimide] |
| 710) | Aminobenzamide (3-ABA) [3-522) |
| | aminobenzamide (3-ABA)] |
| 711) | AMIODARONE |
| 712) | ANANDAMIDE (18:2, n-6) |
| 713) | ANANDAMIDE (20:3, n-6) |
| 714) | ANANDAMIDE (20:4, n-6) |
| 715) | ANANDAMIDE (22:4, n-6) |
| 716) | anisomycin |
| 717) | aphidicolin |
| 718) | ARACHIDONAMIDE |
| 719) | ARACHIDONIC ACID (20:4, n-6) |
| 720) | ARACHIDONOYL-PAF |
| 721) | aristolochic acid |
| 722) | Arvanil |
| 723) | ascomycin (FK-520) |
| 724) | B581 |
| 725) | BADGE |
| 726) | bafilomycin A1 |
| 727) | BAPTA-AM |
| 728) | BAY 11-7082 |
| 729) | BAY K-8644 |
| 730) | BENZAMIL |
| 731) | BEPRIDIL |
| 732) | Bestatin |
| 733) | beta-lapachone |
| 734) | Betulinic acid |
| 735) | bezafibrate |
| 736) | Blebbistatin |
| 737) | BML-190 |
| 738) | Boc-GVV-CHO |
| 739) | bongkrekic acid |
| 740) | brefeldin A |
| 741) | Bromo-7-nitroindazole [3-Bromo-7- |
| | nitroindazole] |
| 742) | Bromo-cAMP [8-Bromo-cAMP] |
| 743) | Bromo-cGMP [8-Bromo-cGMP] |
| 744) | bumetanide |
| 745) | BW-B 70C |
| 746) | C16 CERAMIDE |
| 747) | C2 CERAMIDE |
| 748) | C2 DIHYDROCERAMIDE |
| 749) | C8 CERAMIDE |
| 750) | C8 CERAMINE |
| 750) | C8 DIHYDROCERAMIDE |
| 751) | CA-074-Me |
| 753) | calpeptin |
| 754) | calphostin C |
| 755) | calyculin A |
| 756) | camptothecin |
| 757) | cantharidin |
| 758) | CAPE |
| 759) | capsacin(E) |
| 760) | capsazepine |
| 761) | CARBACYCLIN |
| 762) | castanospermine |
| 763) | CDC |
| 764) | Cerulenin |
| 765) | CGP-37157 |
| 766) | chelerythrine |
| 767) | CIGLITAZONE |
| 768) | CIMATEROL |
| 769) | CinnGEL 2Me |
| 770) | CIRAZOLINE |
| 771) | CITCO |
| 772) | CLOFIBRATE |
| 773) | clonidine |
| 774) | CLOPROSTENOL Na |
| 775) | clozapine |
| 776) | C-PAF |
| 777) | Curcumin |
| 778) | Cycle [Arg-Gly-Asp-D-Phe-Val] |
| 779) | cycloheximide |
| 780) | protein synthesis inhibitor |
| 781) | cycloheximide-N-ethylethanoate |
| 782) | cyclopamine |
| 783) | CYCLOPIAZONIC ACID |
| 784) | cyclosporin A |
| 785) | cypermethrin |
| 786) | cytochalasin B |
| 787) | cytochalasin D |
| 788) | D12-PROSTAGLANDIN J2 |
| 789) | D609 |
| 790) | damnacanthal |
| 791) | DANTROLENE |
| 792) | decoyininc |
| 793) | Decylubiquinone |
| 794) | deoxymannojirimycin(1) |
| 795) | deoxynorjrimycin(1) |
| 796) | Deprenyl |
| 797) | DIAZOXIDE |
| 798) | dibutyrylcyclic AMP |
| 799) | dibutyrylcyclic GMP |
| 800) | DICHLOROBENZAMIL |
| 801) | DIHOMO-GAMMA-LINOLENIC ACID |
| 802) | DIHYDROSPHINGOSINE |
| 803) | DIINDOLYLMETHANE |
| 804) | DILTIAZEM |
| 805) | diphenyleneiodonium C1 |
| 806) | dipyridamole |
| 807) | DL-DIHYDROSPHINGOSINE |
| 808) | DL-PDMP |
| 809) | DL-PPMP |
| 810) | DOCOSAHEXAENOIC ACID (22:6 n-3) |
| 811) | DOCOSAPENTAENOIC ACID |
| 812) | DOCOSATRIENOIC ACID (22:3 n-3) |
| 813) | doxorubicin |
| 814) | DRB |
| 815) | E-4031 |
| 816) | E6 berbamine |
| 817) | E-64-d |
| 818) | Ebselen |
| 819) | EHNA HCl |
| 820) | EICOSA-5,8-DIENOIC ACID (20:2 n-12) |
| 821) | EICOSADIENOIC ACID (20:2 n-6) |
| 822) | EICOSAPENTAENOIC ACID (20:5 n-3) |
| 823) | EICOSATRIENOIC ACID (20:3 n-3) |
| 824) | ENANTIO-PAF C16 |
| 825) | epibatidine (+/−) |
| 826) | etoposide |
| 827) | FARNESYLTHIOACETIC ACID |
| 828) | FCCP |
| 829) | FIPRONIL |
| 830) | FK-506 |
| 831) | FLECAINIDE |
| 832) | FLUFENAMIC ACID |
| 833) | FLUNARIZINE |
| 834) | FLUPROSTENOL |
| 835) | FLUSPIRILINE |
| 836) | FPL-64176 |
| 837) | Fumonisin B1 |
| 838) | Furoxan |
| 839) | GAMMA-LINOLENIC ACID (18:3 n-6) |
| 840) | geldanamycin |
| 841) | genistein |
| 842) | GF-109203X |
| 843) | GINGEROL |
| 844) | Gliotoxin |
| 845) | GLIPIZIDE |
| 846) | GLYBURIDE |
| 847) | GM6001 |
| 848) | Go6976 |
| 849) | GRAYANOTOXIN III |
| 850) | GW-5074 |
| 851) | GW-9662 |
| 852) | H7] |
| 853) | H-89 |
| 854) | H9 |
| 855) | HA-1004 |
| 856) | HA1077 |
| 857) | HA14-1 |
| 858) | HBDDE |
| 859) | Helenalin |
| 860) | Hinokitiol |
| 861) | HISTAMINE |
| 862) | HNMPA-(AM)3 |
| 863) | Hoechst 33342 (cell permeable) |
| | (BisBenzimide) |
| 864) | Huperzine A [(-)-Huperzine A] |
| 865) | IAA-94 |
| 866) | IB-MECA |
| 867) | IBMX |
| 868) | ICRF-193 |
| 869) | Ikarugamyin |
| 870) | Indirubin |
| 871) | Indirubin-3′-monoxime |
| 872) | indomethacin |
| 873) | juglone |
| 874) | K252A |
| 875) | Kavain (+/−) |
| 876) | KN-62 |
| 877) | KT-5720 |
| 878) | L-744,832 |
| 879) | Latrunculin B |
| 880) | Lavendustin A |
| 881) | L-cis-DILTIAZEM |
| 882) | LEUKOTOXIN A (9,10-EODE) |
| 883) | LEUKOTOXIN B (12,13-EODE) |
| 884) | LEUKOTRIENE B4 |
| 885) | LEUKOTRIENE C4 |
| 886) | LEUKOTRIENE D4 |
| 887) | LEUKOTRIENE E4 |
| 888) | Leupeptin |
| 889) | LFM-A13 |
| 890) | LIDOCAINE |
| 891) | LINOLEAMIDE |
| 892) | LINOLEIC ACID |
| 893) | LINOLENIC ACID (18:3 n-3) |
| 894) | LIPOXIN A4 |
| 895) | L-NAME |
| 896) | L-NASPA |
| 897) | LOPERAMIDE |
| 898) | LY-171883 |
| 899) | LY-294002 |
| 900) | LY-83583 |
| 901) | Lycorine |
| 902) | LYSO-PAF C16 |
| 903) | Manoalide |
| 904) | manumycin A |
| 905) | MAPP, D-erythro |
| 906) | MAPP, L-erythro |
| 907) | mastoparan |
| 908) | MBCQ |
| 909) | MCI-186 |
| 910) | MDL-28170 |
| 911) | MEAD ACID (20:3 n-9) |
| 912) | MEAD ETHANOLAMIDE |
| 913) | methotrexate |
| 914) | METHOXY VERAPAMIL |
| 915) | Mevinolin (lovastatin) |
| 916) | MG-132 |
| 917) | Milrinone |
| 918) | MINOXIDIL |
| 919) | MINOXIDIL SULFATE |
| 920) | MISOPROSTOL, FREE ACID |
| 921) | mitomycin C |
| 922) | ML7 |
| 923) | ML9 |
| 924) | MnTBAP |
| 925) | Monastrol |
| 926) | monensin |
| 927) | MY-5445 |
| 928) | Mycophenolic acid |
| 929) | N,N-DIMETHYLSPHINGOSINE |
| 930) | N9-Isopropylolomoucine |
| 931) | N-ACETYL-LEUKOTRIENE E4 |
| 932) | NapSul-Ile-Trp-CHO |
| 933) | N-ARACHIDONOYLGLYCINE |
| 934) | NICARDIPINE |
| 935) | NIFEDIPINE |
| 936) | NIFLUMIC ACID |
| 937) | Nigericin |
| 938) | NIGULDIPINE |
| 939) | Nimesulide |
| 940) | NIMODIPINE |
| 941) | NITRENDIPINE |
| 942) | N-LINOLEOYLGLYCINE |
| 943) | nocodazole |
| 944) | N-PHENYLANTHRANILIC (CL) |
| 945) | NPPB |
| 946) | NS-1619 |
| 947) | NS-398 |
| 948) | NSC-95397 |
| 949) | OBAA |
| 950) | okadaic acid |
| 951) | oligomycin A |
| 952) | olomoucine |
| 953) | ouabain |
| 954) | PAF C16 |
| 955) | PAF C18 |
| 956) | PAF C18:1 |
| 957) | PALMITYLETHANOLAMIDE |
| 958) | Parthenolide |
| 959) | PAXILLINE |
| 960) | PCA 4248 |
| 961) | PCO-400 |
| 962) | PD 98059 |
| 963) | PENITREM A |
| 964) | pepstatin |
| 965) | PHENAMIL |
| 966) | Phenanthridinone [6(5H)-Phenanthridinone] |
| 967) | Phenoxybenzamine |
| 968) | PHENTOLAMINE |
| 969) | PHENYTOIN |
| 970) | PHOSPHATIDIC ACID, DIPALMITOYL |
| 971) | Piceatannol |
| 972) | pifithrin |
| 973) | PIMOZIDE |
| 974) | PINACIDIL |
| 975) | piroxicam |
| 976) | PP1 |
| 977) | PP2 |
| 978) | prazocin |
| 979) | Pregnenolone 16alpha carbonitrile |
| 980) | PRIMA-1 |
| 981) | PROCAINAMIDE |
| 982) | PROPAFENONE |
| 983) | propidium iodide |
| 984) | propranolol (S-) |
| 985) | puromycin |
| 986) | quercetin |
| 987) | QUINIDINE |
| 988) | QUININE |
| 989) | QX-314 |
| 990) | rapamycin |
| 991) | resveratrol |
| 992) | RETINOIC ACID, ALL TRANS |
| 993) | REV-5901 |
| 994) | RG-14620 |
| 995) | RHC-80267 |
| 996) | RK-682 |
| 997) | Ro 20-1724 |
| 998) | Ro 31-8220 |
| 999) | Rolipram |
| 1000) | roscovitine |
| 1001) | Rottlerin |
| 1002) | RWJ-60475-(AM)3 |
| 1003) | RYANODINE |
| 1004) | SB 202190 |
| 1005) | SB 203580 |
| 1006) | SB-415286 |
| 1007) | SB-431542 |
| 1008) | SDZ-201106 |
| 1009) | S-FARNESYL-L-CYSTEINE ME |
| 1010) | Shikonin |
| 1011) | siguazodan |
| 1012) | SKF-96365 |
| 1013) | SP-600125 |
| 1014) | SPHINGOSINE |
| 1015) | Splitomycin |
| 1016) | SQ22536 |
| 1017) | SQ-29548 |
| 1018) | staurosporine |
| 1019) | SU-4312 |
| 1020) | Suramin |
| 1021) | swainsonine |
| 1022) | tamoxifen |
| 1023) | Tanshinone IIA |
| 1024) | taxol = paclitaxel |
| 1025) | TETRAHYDROCANNABINOL-7-OIC ACID |
| 1026) | TETRANDRINE |
| 1027) | thalidomide |
| 1028) | THAPSIGARGIN |
| 1029) | Thiocitrulline [L-Thiocitrulline HCl] |
| 1030) | Thiorphan |
| 1031) | TMB-8 |
| 1032) | TOLAZAMIDE |
| 1033) | TOLBUTAMIDE |
| 1034) | Tosyl-Phe-CMK (TPCK) |
| 1035) | TPEN |
| 1036) | Trequinsin |
| 1037) | trichostatin-A |
| 1038) | trifluoperazine |
| 1039) | TRIM |
| 1040) | Triptolide |
| 1041) | TTNPB |
| 1042) | Tunicamycin |
| 1043) | tyrphostin 1 |
| 1044) | tyrphostin 9 |
| 1045) | tyrphostin AG-126 |
| 1046) | tyrphostin AG-370 |
| 1047) | tyrphostin AG-825 |
| 1048) | Tyrphostin-8 |
| 1049) | U-0126 |
| 1050) | U-37883A |
| 1051) | U-46619 |
| 1052) | U-50488 |
| 1053) | U73122 |
| 1054) | U-74389G |
| 1055) | U-75302 |
| 1056) | valinomycin |
| 1057) | Valproic acid |
| 1058) | VERAPAMIL |
| 1059) | VERATRIDININE |
| 1060) | vinblastine |
| 1061) | vinpocetine |
| 1062) | W7 |
| 1063) | WIN 55,212-2 |
| 1064) | Wiskostatin |
| 1065) | Wortmannin |
| 1066) | WY-14643 |
| 1067) | Xestospongin C |
| 1068) | Y-27632 |
| 1069) | YC-1 |
| 1070) | Yohimbine |
| 1071) | Zaprinast |
| 1072) | Zardaverine |
| 1073) | ZL3VS |
| 1074) | ZM226600 |
| 1075) | ZM336372 |
| 1076) | Z-prolyl-prolinal |
| 1077) | zVAD-FMK |
| 1078) | Ascorbate |
| 1079) | 5-azacytidine |
| 1080) | 5-azadeoxycytidine |
| 1081) | Hexamethylene bisacetamide (HMBA) |
| 1082) | Sodium butyrate |
| 1083) | Dimethyl sulfoxide |
| 1084) | Goosecoid |
| 1085) | Glycogen synthase kinase-3 |
| 1086) | Galectin-1 |
| 1087) | Galectin-3 |

Cell Adhesion Molecules

| | |
|---|---|
| 1086) | Cadherin 1 (E-Cadherin) |
| 1087) | Cadherin 2 (N-Cadherin) |
| 1088) | Cadherin 3 (P-Cadherin) |
| 1089) | Cadherin 4 (R-Cadherin) |
| 1090) | Cadherin 5 (VE-Cadherin) |
| 1091) | Cadherin 6 (K-Cadherin) |
| 1092) | Cadherin 7 |
| 1093) | Cadherin 8 |
| 1094) | Cadherin 9 |
| 1095) | Cadherin 10 |
| 1096) | Cadherin 11 (OB-Cadherin) |
| 1097) | Cadherin 12 (BR-Cadherin) |
| 1098) | Cadherin 13 (H-Cadherin) |
| 1099) | Cadherin 14 (same as Cadherin 18) |
| 1100) | Cadherin 15 (M-Cadherin) |
| 1101) | Cadherin 16 (KSP-Cadherin) |
| 1102) | LI Cadherin |

Culture Media

| | |
|---|---|
| 1103) | DMEM (Dulbecco's Modified Eagle's Medium). |
| | HyClone Cat. No. SH30285.03 |
| 1104) | Airway Epithelial Growth Medium (PromoCell |
| | Cat. No. C-21260 with supplement Cat No. |
| | C-39160) |
| 1105) | Epi-Life (LSGS) Medium (Cascade Cat. No. |
| | M-EPIcf/PRF-500 with supplement Cat. No. |
| | S-003-10) |
| 1106) | Neural Basal Medium B-27 (Gibco Cat. No. |
| | 12348-017 with B-27 supplement Cat. No. |
| | 12587-010) |
| 1107) | Neural Basal Medium N-2 (Gibco Cat. No. |
| | 12348-017 with N-2 supplement Cat. No. |
| | 17502-048) |
| 1108) | HepatoZyme-SFM (Gibco Cat. No. 17705-021) |
| 1109) | Epi-Life (HKGS) Medium (Cascade Cat. No. M |
| | EPIcf/PRF-500 with supplement Cat. No. |
| | S-001-5) |
| 1110) | Endothelial Cell Growth Medium (PromoCell |
| | Cat. No. C-22221 with supplement Cat No. |
| | C-39221) |
| 1111) | Endothelial Cell SFM (Gibco Cat. No. |
| | 11111-044 with basic fibroblast growth |
| | factor Cat. No. 13256-029, epidermal growth |
| | factor Cat. No. 13247-051 and fibronectin |
| | Cat. No. 33016-015) |
| 1112) | Skeletal Muscle Medium (PromoCell Cat No. |
| | C-23260 with supplement Cat No. C-39360) |
| 1113) | Smooth Muscle Basal Medium (PromoCell Cat. |
| | No. C-22262 with supplement Cat. No. |
| | C-39262) |
| 1114) | MesenCult Medium (Stem Cell Technologies Cat |
| | No. 05041 with supplement Cat. No. 05402) |
| 1115) | Melanocyte Growth Medium (PromoCell Cat. No. |
| | C 24010 with supplement Cat. No. C-39410) |
| 1116) | Ham's F-10 Medium |
| 1117) | Ham's F-12 Medium |
| 1118) | DMEM/Ham's F-12 50/50 mix |
| 1119) | Iscove's Modified Dulbecco's Medium (IMDM) |
| 1120) | Leibovitz's L-15 Medium |
| 1121) | McCoy's 5A Medium Modified |
| 1122) | RPMI 1640 Medium |
| 1123) | Glasgow's MEM (GMEM) |
| 1124) | Eagle's Medium |
| 1125) | Medium 199 |
| 1126) | MEM Eagle-Earle's |

Antibiotics

| | |
|---|---|
| 1127) | Penicillin |
| 1128) | Streptomycin |
| 1129) | Gentamycin |
| 1130) | Neomycin |
| 1131) | G418 |

Other Factors

| | |
|---|---|
| 1132) | Human plasma |
| 1133) | Chick embryo extract |
| 1134) | Human plasmanate |

TABLE II
Differentiated Cells and Tissues

```
Heart
  1) Ventricular myocardium
  2) Auricular myocardium
  3) Sinus node myocardium
  4) anterior, middle and posterior internodal tracts
  5) atrioventricular (AV) node
  6) His bundle
  7) right and left bundle branches
  8) anterior-superior and posterior-inferior divisions of the left bundle
  9) The Purkinje network
Musculo-Skeletal
10) Cartilage - Hyaline
11) Cartilage - Elastic
12) Cartilage - Fibrous
13) Bone - compact
14) Bone - cancellous
15) Intervertebral disc
16) Skeletal muscle
Nervous Tissues
17) Dopaminergic neurons of the substantia nigra
18) Autonomic - Parasympathetic
19) Autonomic - Sympathetic
20) Schwann cells
20) Cranial nerves
21) Myelinating - Schwann cells
22) Motor neurons
27) Outer neuroblastic layer of the developing retina
28) Inner neuroblastic layer of the developing retina
29) Outer nuclear layer of the retina
30) Outer plexiform layer of the retina
31) Inner nuclear layer of the retina
32) Inner plexiform layer of the retina
33) Ganglion cell layer of the retina
34) Thalamus
35) Hippocampus
36) Hypothalamus
37) Cerebral cortex
Respiratory System
38) Trachea
39) Tracheobronchial epithelium
40) Brochi
41) Lungs
42) Type I pneumocytes
43) Type II pneumocytes
Endocrine System
44) Pancreatic beta cells
45) Anterior pituitary
46) Neural pituitary
46) Adrenal cortex
47) Adrenal medulla
48) Thyroid gland
49) Parathyroid gland
Vascular System
50) Aorta
51) Pulmonary vein
52) capillaries
53) Vascular endothelium
54) Vascular smooth muscle
55) Pericytes
56) Adventitial cells
Hematopoietic system
55) Hematopoietic stem cells
56) Lymphoid progenitors
57) B lymphocytes
58) T lymphocytes
59) Myeloid progenitors
Integumentary system
60) Dermis
61) Epidermis
62) Hair follicles
63) Sebaceous glands
63) Sweat glands
64) Subcutaneous adipose tissue
Urinary System
65) Kidney
66) Renal tubule epithelial cells
67) Renal cortex
68) Ureters
69) Bladder
70) Urethra
Gastrointestinal system
71) Oral epithelium
72) Cheek epithelium
72) Teeth
72) Esophagus
72) Gastric mucosa
73) Jejunum
74) Ileum
75) Duodenum
76) Colon
77) Pancreas
78) Hepatic parenchymal cells
79) Hepatic Stellate (Ito) cells
Sensory systems
79) Olfactory epithelium
24) Inner ear
25) Lens
26) Cornea
23) Sensory neurons
25) Eye
26) Retinal pigment epithelium
```

TABLE III
Differentiating Cell Types (includes SPF chick embryonic tissues,
nonhuman animal embryonic/fetal cells and
tissues, and human embryonic/fetal cells and tissues

```
Endoderm - Embryonic
  1) Definitive endodermal (entodermal) cells
  2) Foregut endodermal cells
  3) Midgut endodermal cells
  4) Hindgut endodermal cells
  5) Ventral pancreatic bud cells
Mesoderm - Embryonic
  6) Intraembryonic mesodermal cells
  7) Prechordal plate mesodermal cells
  8) Notochordal plate mesodermal cells
  9) Notochord mesodermal cells
10) Paraxial mesodermal cells
11) Intermediate mesodermal cells
12) Lateral plate mesodermal cells
13) Splanchnopleuiric mesodermal cells
14) Somatopleuric mesodermal cells
15) Somitomeric mesodermal cells
16) Somite mesodermal cells
17) Cervical somite mesodermal cells
18) Thoracic somite mesodermal cells
19) Lumbar somite mesodermal cells
20) Sacral somite mesodermal cells
21) Sclerotome mesodermal cells
22) Myotome mesodermal cells
23) Epimere myotome mesodermal cells
24) Hypomere myotome mesodermal cells
25) Dermatome mesodermal cells
26) Angioblasts
27) Mural progenitor cells
28) Vascular smooth muscle cells
29) Pericytes
30) Myoepithelial cells
31) Enteric (intestinal) smooth muscle cells
32) Limb bud mesenchyme
33) Osteoblasts
34) Synoviocytes
35) Hemangioblasts
36) Angioblasts
37) Skeletal muscle myoblasts
38) cardiogenic mesoderm
39) Endocardial primordial cells
40) Epi-myocardial primordial cells
41) Dorsal mesocardial cells
Ectoderm - Embryonic
42) cranial neural crest
43) cardiac neural crest
44) vagal neural crest
45) trunk neural crest
Extraembryonic Cells
46) Hypoblast (primary endoderm)
47) Extraembryonic endodermal cells
49) Amnioblasts
49) Syncytiotrophoblasts
50) Cytotrophoblasts
51) Extraembryonic mesodermal cells
```

TABLE IV
Teratogens

Abovis
Acebutolol
Acebutolol hydrochloride
Acemetacin
Acepreval
Acetaldehyde
Acetamide
5-Acetamide-1,3,4-thiadiazole-2-sulfonamide
Acetazolamide sodium
Acetic acid methylnitrosaminomethyl ester
Acetohydroxamic acid
Acetonitrile
3-(alpha-Acetonyl-para-nitrobenzyl)-4-hydroxy-coumarin
para-Acetophenetidide
17-Acetoxy-19-nor-17-alpha-pregn-4-EN-20-YN-3-one
Acetoxyphenylmercury
Acetoxytriphenylstannane
1-alpha-Acetylmethadol hydrochloride
Acetylsalicylic acid
Acetyltryptophan
Acid red 92
4,-(9-Acridinylamino) methanesulphon-meta-anisidide
Acriflavin hydrochloride
Acrylic acid
Acrylonitrile
Actihaemyl
Actinomycin
Actinomycin C
Actinomycin D
Acyclovir
Acyclovir sodium salt
Adalat
1-Adamantanamine hydrochloride
Adapin
Adenine
Adenosine-3,-(alpha-amino-para-methoxyhydrocinnamamido)-3,-deoxy-n,n-dimethyl
Adipic acid bis (2-ethylhexyl) ester
Adipic acid dibutyl ester
Adipic acid di(2-hexyloxyethyl) ester
Adobiol
Adona trihydrate
1-Adrenaline chloride
Adrenocorticotrophic hormone
Adriamycin
Aflatoxin
Aflatoxin B1
Afridol blue
Agent orange
Alclometasone dipropionate
Alcohol sulphate
Aldactazide
Aldecin
Aldimorph
Aldrin
alpha-Alkenesulfonic acid
Alkyl dimethylbenzyl ammonium chloride
3-(Alkylamino) propionitrile
Alkylbenzenesulfonate
Allantoxanic acid, potassium salt
Alloxan
Allyl chloride
Allyl glucosinolate
Allyl isothiocyanate
6-Allyl-6,7-dihydro-5h-dibenz (c,e) azepine phosphate
Allylestrenol
(4-Allyloxy-3-chlorophenyl)acetic acid
Alternariol
Alternariol monomethyl ether and alternariol (1:1)
Alternariol-9-methyl ether
Aluminum aceglutamide
Aluminum chloride
Aluminum chloride hexahydrate
Aluminum lactate
Aluminium (III) nitrate, nonahydrate (1:3:9)
Aluminium potassium sulfate, dodecahydrate
Ambroxol hydrochloride
Ametycin
Amfenac sodium monohydrate
Amicardine
N1-Amidinosulfanilamide
Amidoline
5-((2-Aminoacetamido) methyl)-1-(4-chloro-2-(orthochlorobenzoyl) phenyl)-n,n-dimethyl-1H-S-triazole-3-carboxamide,
hydrochloride, dihydrate
Aminoacetonitrile bisulfate
Aminoacetonitrile sulfate
2-Aminobenzimidazole
2-Amino-6-benzimidazolyl phenylketone
Aminobenzylpenicillin
5-Amino-1-bis (dimethylamide) phosphoryl-3-phenyl-1,2,4-triazole
2-Amino-5-bromo-6-phenyl-4 (1h)-pyrimidinone
4-Amino-2-(4-butanoylhexahydro-1h-1,4-diazepin-1-yl)-6,7-dimethoxyquinazoline hydrochloride
2-Amino-5-butylbenzimidazole
5-Amino-1,6-dihydro-7h-v-triazolo (4,5-d) pyrimidin-7-one
3-(2-aminoethyl) indol-5-ol
3-(2-aminoethyl) indol-5-ol creatinine sulfate
trans-4-Aminoethylcyclohexane-1-carboxylic acid
Aminoglutethimide
2-Amino-3-hydroxybenzoic acid
8-Amino-7-hydroxy-3,6-napthalenedisulfonic acid, sodium salt
4-Amino-n-(6-methoxy-3-pyridazinyl)-benzenesulfonamide
3-Amino-4-methylbenzenesulfonylcyclohexylurea
2-Amino-6-(1,-methyl-4,-nitro-5,-imidazolyl) mercaptopurine
1-(4-Amino-2-methylpyrimidin-5-yl)methyl-3-(2-chloroethyl)-3-nitrosourea
2-Amino-4-(methylsulfinyl) butyric acid
5-Amino-2-napthalenesulfonic acid sodium salt
6-Aminonicotinamide
2-Amino-4-nitroaniline
4-Amino-2-nitroaniline
Aminonucleoside puromycin
2-Aminophenol
3-Aminophenol
4-Aminophenol
meta-Aminophenol, chlorinated
7-(d-alpha-aminophenylacetamido) desacetoxycephalosporanic acid
3-Aminopropionitrile
beta-Aminopropionitrile fumarate
Aminopropyl aminoethylthiophosphate
3-(2-Aminopropyl) indole
Aminopteridine
2-Aminopurine-6-thiol
Aminopyrine sodium sulfonate
Aminopyrine-barbital
5-Amino-2-beta-d-ribofuranosyl-as-triazin-3-(2H)-one
4-Amino-2,2,5,5-tetrakis (trifluoromethyl)-3-imidazoline
2-Amino-1,3,4-thiadiazole
2-Amino-1,3,4-thiadiazolehydrochloride
2-Amino-1,3,4-thiadiazole-5-sulfonamide sodium salt
1-Amino-2-(4-thiazolyl)-5-benzimidazolecarbamic acid isopropyl ester
Amitriptyline-n-oxide
Amitrole
Ammonium vanadate
Amosulalol hydrochloride
Amoxicillin trihydrate
dl-Amphetamine sulfate
Ampicillin trihydrate
Amrinone
Amsacrine lactate
Amygdalin
Anabasine
Anatoxin I
Androctonus amoreuxi venom
Androfluorene
Androfurazanol
Androstanazol
Androstenediol dipropionate
Androstenedione
Androstenolone
Androstestone-M
Angel dust
Angiotonin
Anguidin
Aniline violet
6-(para-anilinosulfonyl) metanilamide
2-Anthracenamine
Antibiotic BB-K8
Antibiotic BB-K8 sulfate
Antibiotic BL-640
Antibiotic MA 144A1
Antimony oxide
Apholate
9-beta-d-Arabino furanosyl adenine
Arabinocytidine
Ara-C palmitate
Araten phosphate
Arathane
1-Arginine monohydrochloride
Aristocort
Aristocort acetonide
Aristocort diacetate
Aristolic acid
Aristospan
Aromatol
Arotinoic acid
Arotinoic methanol
Arotinoid ethyl ester
Arsenic
ortho-Arsenic acid
Arsenic acid, disodium salt, heptahydrate
Arsenic acid, sodium salt
Arsenic trioxide
Asalin
1-Ascorbic acid
1-Asparaginase
Atrazine
Atromid S
Atropine
Atropine sulfate (2:1)
Auranofin
Aureine
1-Aurothio-d-glucopyranose
Ayush-47
Azabicyclane citrate
Azactam
Azacytidine
Azaserine
Azathioprine
Azelastine hydrochloride
1-2-Azetidinecarboxylic acid
Azinphos methyl
Azo blue
Azo ethane
Azosemide
Azoxyethane
Azoxymethane
Baccidal
Bacmecillinam
Bal
Barbital sodium
Barium ferrite
Barium fluoride
Bayer 205
Baythion
Befunolol hydrochloride
Bendacort
Bendadryl hydrochloride
Benedectin
Benomyl
Benzarone
d-Benzedrine sulfate
Benzenamine hydrochloride
Benzene
Benzene hexachloride-g-isomer
1-Benzhydryl-4-(2-(2-hydroxyethoxy)ethyl)piperazine
Benzidamine hydrochloride
2-Benzimidazolecarbamic acid
1-(2-Benzimidazolyl)-3-methylurea
1,2-Benzisothiazol-3 (2H)-one-1,1-dioxide
1,2-Benzisoxazole-3-methanesulfonamide
Benzo (alpha) pyrene
Benzo (e) pyrene
Benzoctamine hydrochloride
para-Benzoquinone monoimine
Benzothiazole disulfide
2-Benzothiazolethiol
2-Benzothiazolyl-N-morpholinosulfide
2-(meta-Benzoylphenyl) propionic acid
2-Benzylbenzimidazole
Benzyl chloride
Benzyl penicillinic acid sodium salt
Beryllium chloride
Beryllium oxide
Bestrabucil
Betamethasone
Betamethasone acetate and betamethasone phosphate
Betamethasone benzoate
Betamethasone dipropionate
Betamethasone disodium phosphate
Betel nut
Betnelan phosphate
BHT (food grade)
Bindon ethyl ether
Binoside
4-Biphenylacetic acid
2-Biphenylol
2-Biphenylol, sodium salt
3-(4-Biphenylylcarbonyl) propionic acid
2,2-Bipyridine
Bis(para-acetoxyphenyl)-2-methylcylcophexylidenemethane
4,4-Bis(1-amino-8-hydroxy-2,4-disulfo-7-napthylazo)-3,3,-bitolyl,tetrasodium salt
1,4-Bis(3-bromopropionyl)-piperazine
1,3-Bis(carbamoylthio)-2-(N,N-dimethylamino)propane hydrochloride
trans-N,N,-Bis(2-chlorobenzyl)-1,4 cyclohexanebis (methylamine) dihydrochloride
Bis(2-chloroethyl) amine hydrochloride
4,-(Bis (2-chloroethyl) amino) acetanilide
4,-(Bis (2-chloroethyl) amino)-2-fluoro acetanilide
dl-3-(para-(Bis (2-chloroethyl) amino) phenyl)alanine
Bis(beta-chloroethyl) methylamine
Bis(2-chloroethyl) methylamine hydrochloride
Bis (2-chloroethyl) sulfide
N,N,-Bis (2-chloroethyl)-N-nitrosourea
N,N,-Bis (2-chloroethyl)-para-phenylenediamine
Bis (para-chlorophenyl) acetic acid
2,2-Bis (ortho, para-chlorophenyl)-1,1,1-trichloroethane
1,1-Bis (para-chlorophenyl)-2,2,2-trichloroethanol
Bis (beta-cyanoetyl) amine
Bis (dichloroacetyl)-1,8-diaminooctane
3,5-Bis-dimethylamino-1,2,4-dithiazolium chloride
Bis (dimethyldithiocarbamato) zinc
(((3,5-Bis(1,1-dimethylethyl)-4-hydroxyphenyl)methyl)thio)acetic acid 2-ethylhexyl ester
Bis (dimethylthiocarbamoyl) sulfate
2,4-Bis (ethylamino)-6-chloro-s-triazine
Bis (ethylmercuri) phosphate
Bis-HM-A-TDA
Bishydroxycoumarin
Bis (4-hydroxy-3-coumarin) acetic acid ethyl ester
1,4-Bis ((2-((2-hydroxyethyl) amino) ethyl) amino)-9,10-athracenedione diacetate
Bis (isooctyloxycarbonylmethylthio) dioctyl stannane
Bis (2-methoxy ethyl) ether
Bisphenol A
1,4-Bis (phenyl amino) benzene
Bis (tributyl tin) oxide
2-(3,5-Bis (trifluoromethyl) phenyl)-N-methyl-hydrazinecarbothioamide (9CI)
Bladex
Bleomycin sulfate
Bomt
Bracken fern, dried
Bradykinin
Bredinin
Bremfol
Bromacil
Bromazepam
Bromocriptine
Bromocriptine mesilate
5-Bromo-2,-deoxyuridine
2-Bromo-d-lysergic acid diethylamide
6-Bromo-1,2-napththoquinone
Bromoperidol
Bromophenophos
4-Bromophenyl chloromethyl sulfone
Buclizine dihydrochloride
Budesonide
Bunitrolol hydrochloride
Buprenorphine hydrochloride
1,3-Butadiene
Butamirate citrate
1,4-Butanediamine
1,4-Butanediol dimethyl sulfonate
4-Butanolide
Butobarbital
Butoctamide semisuccinate
Butorphanol tartrate
Butoxybenzyl hyoscyamine bromide
2-Butoxyethanol
para-Butoxyphenylacetohydroxamic acid
Butriptyline
Bromoperidol
Bromophenophos
4-Bromophenyl chloromethyl sulfone
Buclizine dihydrochloride
Budesonide
Bunitrolol hydrochloride
Buprenorphine hydrochloride
1,3-Butadiene
Butamirate citrate
1,4-Butanediamine
1,4-Butanediol dimethyl sulfonate
4-Butanolide
Butobarbital
Butoctamide semisuccinate
Butorphanol tartrate
Butoxybenzyl hyoscyamine bromide
2-Butoxyethanol
para-Butoxyphenylacetohydroxamic acid
Butriptyline
n-Butyl acetate
n-Butyl alcohol
sec-Butyl alcohol
tert-Butyl alcohol
alpha,-((tert-Butyl amino) methyl)-4-hydroxy-meta-xylene-alpha,alpha-diol
Butyl carbamate
Butyl carbobutoxymethyl phthalate
Butyl dichlorophenoxyacetate
Butyl ethyl acetic acid
Butyl flufenamate
n-Butyl glycidyl ether
n-Butyl mercaptan
n-Butyl-3,ortho-acetyl-12-b-13-alpha-dihydrojervine
1-(tert-Butylamino)-3-(2-chloro-5-methylphenoxy)-2-propanol hydrochloride
alpha-Butylbenzenemethanol
5-Butyl-2-benzimidazolecarbamic acid methyl ester
5-Butyl-1-cylcohexylbarbituric acid
2-sec-Butyl-4,6-dinitrophenol
4-Butyl-1,2-diphenyl-3,5-dioxo pyrazolidine
n-Butyl-N-nitroso-1-butamine
N-Butyl-N-nitroso ethyl carbamate
n-Butylnitrosourea
1-Butyl-2′,6′-pipecoloxylidide
1-Butyl-3-sulfanilyl urea
1-Butyl-3-(para-tolyl sulfonyl) urea
1-Butyl-3-(para-tolylsulfonyl) urea, sodium salt
Butyl-2,4,5-trichlorophenoxyacetate
1-Butyryl-4-(phenylallyl) piperazine hydrochloride
Buzepide methiodide
Cadmium
Cadmium (II) acetate
Cadmium chloride
Cadmium chloride, dihydrate
Cadmium compounds
Cadmium oxide
Cadmium sulfate (1:1)
Cadmium sulfate (1:1) hydrate (3:8)
Cadralazine
Caffeic acid
Caffeine
Calcium EbrA complex
Calcium fluoride
Calcium phosphonomycin hydrate
Calcium trisodium diethylene triamine pentaacetate
Calcium valproate
Calcium-N-2-ethylhexyl-beta-oxybutyramide semisuccinate
Cambendazole
Camphorated oil
Candida albicans glycoproteins
Cannabidiol
Cannabinol
Cannabis
Cap
Caprolactam
Captafol
Captan
Carbamates
Carbaryl
Carbendazim and sodium nitrite (5:1)
Carbidopa
Carbinilic acid isopropyl ester
Carbofuran
Carbon dioxide
Carbon disulfide
Carbon monoxide
Carbon tetrachloride
Carboprost tromethamine
Cargutocin
Carmetizide
Carmofur
1-Carnitine hydrochloride
Carnosine
Carzinophilin
Cassava, manihot utilissima
Catatoxic steroid No. 1
d-Catechol
CAZ pentahydrate
Cefamandole sodium
Cefotaxime sodium
Cefazedone
Cefazolin sodium salt
Cefmetazole
Cefmetazole sodium
Cefroxadin
Cefuroxim
Celestan-depot
Cellryl
Cellulose acetate monophthalate
Centbucridine hydrochloride
Centchroman
Cephalothin
Cervagem
Cesium arsenate
Cethylamine hydrofluoride
alpha-Chaconine
Chenodeoxycholic acid
Chlodithane
Chlorambucil
Chloramphenicol
Chloramphenicol monosuccinate sodium salt
Chloramphenicol palmitate
Chlorcyclizine hydrochloride
Chlorcyclizine hydrochloride A
Chlorcyclohexamide
Chlordane
Chlorimipramine
Chlorinated camphene
Chlorinated dibenzo dioxins
Chlorisopropamide
Chlormadinon
para-Chloro dimethylaminoazobenzene
2-Chloroadenosine
1-(3-Chloroallyl)-3,5,7-triaza-1-azoniaadamantane chloride
3-Chloro-4-aminoaniline
1-((para-(2-(Chloro-ortho-anisamido)ethyl)phenyl)sulfonyl)-3-cylcohexyl urea
Chlorobenzene
ortho-Chlorobenzylidene malononitrile
1-para-Chlorobenzyl-1H-indazole-3-carboxylic acid
7-Chloro-5-(ortho-chlorophenyl)-1,3-dihydro-3-hydroxy-2H-1,4-benzodiazepin-2-one
Chlorocylcine
6-Chloro-5-Cyclohexyl-1-indancarboxylic acid
6-Chloro-5-(2,3-dichlorophenoxy)-2-methylthio-benzimidazole
5-Chloro-2-(2-(diethylamino)ethoxy)benzanilide
7-Chloro-1,3-dihydro-5-phenyl,2H-1,4-benzodiazepin-2-one
Chloroethyl mercury
1-(2-Chloroethyl)-3-cylcohexyl-1-nitrosourea
1-Chloro-3-ethyl-1-penten-4-YN-3-OL
Chloroform
4-Chloro-N-furfuryl-5-sulfamoylanthranilic acid
Chlorogenic acid
endo-4-Chloro-N-(hexahydro-4,7-methanoisoindol-2-YL)-3-sulfamoylbenzamide
(−)-N-((5-Chloro-8-hydroxy-3-methyl-1-OXO-7-isochromanyl) carbonyl)-3-phenylalanine
5-Chloro-7-iodo-8-quinolinol
(4-Chloro-2-methylphenoxy) acetic acid
2-(4-Chloro-2-methylphenoxy) propanoic acid (R) (9CI)
4-Chloro-2-methylphenoxy-alpha-propionic acid
7-Chloro-1-methyl-5-phenyl-1H-1,5-benzodiazepine-2,4(3H,5H)-dione
2-Chloro-11-(4-methylpiperazino) dibenzo (b,f) (1,4) thiazepine
4-((5-Chloro-2-OXO-3(2H)-benzothiazolyl)acetyl)-1-piperazineethanol
4-(3-(2-Chlorophenothiazin-10-YL)propyl)-1-piperazineethanol
4-Chlorophenylalanine
1-(para-Chloro-alpha-phenylbenzyl)-4-(2-((2-hydroxyethoxy) ethyl)piperazine)
1-(meta-Chlorophenyl)-3-N,N-dimethylcarbamoyl-5-methoxypyrazole
3-(para-Chlorophenyl)-1,1,dimethylurea
5,(2-Chlorophenyl)-7-ethyl-1-methyl-1,3-dihydro-2H-thieno (2,3-e) (1,4) diazepin-2-one
N-3-Chlorophenylisopropylcarbamate
3-(4-Chlorophenyl)-1-methoxy-1-methylurea
2-(ortho-Chlorophenyl)-2-(methylamino)cyclohexanone hydrochloride
3-(para-Chlorophenyl)-1-methyl-1-(1-methyl-2-propynyl) urea
4-(para-Chlorophenyl)-2-phenyl-5-thiazoleacetic acid
1-(para-Chlorophenylsulfonyl)-3-propylurea
para-Chlorophenyl-2,4,5-trichlorophenyl sulfone
4-Chlorophenyl-2,4,5-trichlorophenylazosulfide mixed with 1,1-bis(4-chlorophenyl)ethanol
Chloropromazine
Chloropromazine hydrochloride
Chloroquine
Chloroquine diphosphate
N-(3-Chloro-ortho-tolyl) anthranilic acid
2-((4-Chloro-ortho-tolyl)oxy)propionic acid potassium salt
Chloro(triethylphosphine)gold
Chlorovinylarsine dichloride
4-Chloro-3,5-xylenol
Chlorphentermine
g-(4-(para-Chlorphenyl)-4-hydroxiperidino)-para-fluorbutyrophenone
Cholecalciferol
Cholesterol
Cholestyramine
Chorionic gonadotropin
Chromium chloride
Chromium (VI) oxide (1:3)
Chromium trichloride hexahydrate
Chromomycin A3
C.I. 45405
C.I. Direct blue 1, tetrasodium salt
C.I. Direct blue 6, tetrasodium salt
C.I. Direct blue 14, tetrasodium salt
C.I. Direct blue 15, tetrasodium salt
Cilostazol
Cinoxacin
Citreoviridin
Citrinin
Citrus hystrix DC., fruit peel extract
Clavacin
Clindamycin-2-palmitate monohydrochloride
Clindamycin-2-phosphate
Cloazepam
Clobetasone butyrate
Cloconazole hydrochloride
Clofedanol hydrochloride
Clofexamide phenylbutazone
Clomiphene
racemic-Clomiphene citrate
trans-Clomiphene citrate
Clonidine hydrochloride
Clonixic acid
Cloxazolazepam
Clozapine
Coagulase
Cobalt (III) acetylacetonate
Cobalt (II) chloride
Corn oil
Corticosterone
Corticosterone acetate
Cortisol
Cortisone
Cortisone-21-acetate
Cottonseed oil (unhydrogenated)
Coumarin
Cravetin
meta-Cresol
Cumoesterol
S-1-Cyano-2-hydroxy-3-butene
Cyanotrimethylandrostenolone
Cycasin
Cyclocytidine hydrochloride
Cycloguanyl
Cyclohexanamine hydrochloride
Cycloheximide
Cyclohexylamine
Cyclohexylamine sulfate
2-(Cyclohexylamino)ethanol
N-Cyclohexyl-2-benzothiazolesulfenamide
4-(4-Cyclohexyl-3-chlorophenyl)-4-oxobutyric acid
1-Cyclohexyl-3-para-tolysulfonylurea
Cyclonite
Cyclopamine
Cyclophosphamide hydrate
Cyclophosphoramide
alpha-Cyclopiazonic acid
5-(Cyclopropylcarbonyl)-2-benzimidazolecarbamic acid methyl ester
Cyprosterone acetate
Cysteine-germanic acid
Cytochalasin B
Cytochalasin E
Cytostasan
Cytoxal alcohol
Cytoxyl amine
Demeton-O + Demeton-S
Demeton-O-methyl
Demetrin
Denopamine
11-Deoxo-12-beta,13-alpha-dihydro-11-alpha-hydroxyjervine
11-Deoxojervine-4-EN-3-one
2,-Deoxy-5-fluorouridine
2-Deoxyglucose
2,-Deoxy-5-iodouridine
4-Deoxypyridoxol hydrochloride
Dephosphate bromofenofos
Depofemin
Depo-medrate
N-Desacetylthiocolchicine
Desoxymetasone
2-Desoxyphenobarbital
Detergents, Liquid containing AES
Detergents, Liquid containing LAS
Dexamethasone acetate
Dexamethasone 17,21-dipropionate
Dexamethasone palmitate
Dextran 1
Dextran 70
Dextropropoxyphene napsy
alpha-DFMO
Diabenor
Diacetylmorphine hydrochloride
Dialifor
Diamicron
2,4-Diamino-6-methyl-5-phenylpyrimidine
2,4-Diamino-5-phenyl-6-ethylpyrimidine
2,4-Diamino-5-phenyl-6-propylpyrimidine
2,4-Diamino-5-phenylpyrimidine
2,5-Diaminotoluene dihydrochloride
Diazepam
Diazinon
6-Diazo-5-oxonorleucine
Diazoxide
Dibekacin
5H-Dibenz (b,f) azepine-5-carboxamide
5H-Dibenz (b,f) azepine, 3-chloro-5-(3-(4-carbamoyl-4-piperidinopiperine
Dibenz (b,f) (1,4) oxazepine
Dibenzacepin
Dibenzyline hydrochloride
1,2-Dibromo-3-chloropropane
3,5-Dibromo-4-hydroxyphenyl-2-ethyl-3-benzofuranyl ketone
Dibromomaleinimide
1,6-Dibromomannitol
Dibutyl phthalate
N,N-Di-n-butylformamide
Dibutyryl cyclic amp
Dicarbadodecaboranylmethylethyl sulfide
Dicarbadodecaboranylmethylpropyl sulfide
1-(2,4-Dichlorbenzyl)indazole-3-carboxylic acid
Dichloroacetonitrile
(ortho-((2,6-Dichloroanilino)phenyl) acetic acid sodium salt
ortho-Dichlorobenzene
para-Dichlorobenzene
4,5-Dichloro-meta-benzenedisulfonamide
2,2,-Dichlorobiphenyl
Dichloro-1,3-butadiene
1,4-Dichloro-2-butene
2,2-Dichloro-1,1-difluorethyl methyl ether
5,5-Dichloro-2,2,-dihydroxy-3,3,-dinitrobiphenyl
1,1-Dichloroethane
2,3-Dichloro-N-ethylmaleinimide
Dichloromaleimide
Dichloro-N-methylmaleimide
2,4-Dichloro-4,-nitrodiphenyl ether
2,4-Dichlorophenol
(2,4-Dichlorophenoxy) acetic acid butoxyethyl ester
(2,4-Dichlorophenoxy) acetic acid dimethylamine
4-(2,4-Dichlorophenoxy) butyric acid
2-(2,4-Dichlorophenoxy) propionic acid
(+)-2-(2,4-Dichlorophenoxy) propionic acid
3,4-Dichlorophenoxyacetic acid
2,4-Dichlorophenoxyacetic acid propylene glycol butyl ether ester
2-(2,6-Dichlorophenylamino)-2-imidazoline
3,6-Dichloro-2-pyridinecarboxylic acid
Dichlorvos
Dicyclohexyl adipate
Dicyclohexyl-18-crown-6
Dicyclopentadienyldichlorotitanium
7,8-Didehydroretinoic acid
Dieldrin
Diethyl carbitol
Diethyl carbonate
Diethyl mercury
Diethyl phthalate
Diethyl sulfate
2-(Diethylamino)-2′,6′-acetoxylidide
2-Diethylamino-2′,6′-acetoxylidide hydrochloride
ortho-(Diethylaminoethoxy) benzanilide
2-(2-(Diethylamino)ethoxy)-5-bromobenzanilide
2-(2-(Diethylamino)ethoxy)-2,-chloro-benzanilide
2-(2-(Diethylamino)ethoxy)-3,-chloro-benzanilide
2-(2-(Diethylamino)ethoxy)-3,-chloro-methylbenzanilide
(para-2-Diethylaminoethoxyphenyl)-1-phenyl-2-para-anisylethanol
1-(2-(Diethylamino)ethyl)reserpine
7-Diethylamino-5-methyl-s-triazolo(1,5-alpha) pyrimidine
N,N-Diethylbenzenesulfonamide
Diethylcarbamazine
Diethylcarbamazine acid citrate
Diethyldiphenyl dichloroethane
Diethylene glycol
Diethylene glycol monomethyl ether
1,2-Diethylhydrazine
1,2-Diethylhydrazine dihydrochloride
N,N-Diethyllsergamide
N,N-Diethyl-4-methyl-3-oxo-5-alpha-4-azaandrostane-17-beta-carboxamide
3,3-Diethyl-1-(meta-pyridyl)triazene
a,a-Diethyl-(E)-4,4,-stilbenediol bis(dihydrogen phosphate)
a,a-Diethyl-4,4,-stilbenediol disodium salt
Diethylstilbesterol
Diethylstilbestrol dipalmitate
Diethylstilbestrol dipropionate
Diflorasone diacetate
Diflucortolone valerate
dl-alpha-Difluoromethylornithine
5-(2,4-Difluorophenyl) salicylic acid
Difluprednate
Digoxin
Dihydantoin
Dihydrocodeinone bitartrate
Dihydrodiethylstilbestrol
3,4-Dihydro-6-(4-(3,4-dimethoxybenzoyl)-1-piperazinyl)-2(1H)-quinolinone
5,6-Dihydro-N-(3-(dimethylamino)propyl)-11H-dibenz(b,e)azepine
10,11-Dihydro-5-(3-(dimethylamino)propyl)-5H-dibenz(b,f)azepine hydrochloride
5,6-Dihydro-para-dithiin-2,3-dicarboximide
12,b,13,alpha-Dihydrojervine
10,11-Dihydro-5-(3-(methylamino)propyl)-5H-dibenz(b,f)azepine hydrochloride
1,7-Dihydro-6H-purin-6-one
7,8-Dihydroretinoic acid
Dihydrostreptomycin
4-Dihydrotestosterone
3-alpha,17-beta-Dihydroxy-5-alpha-androstane
3-alpha,7-beta-Dihydroxy-6-beta-cholan-24-OIC acid
1 alpha,25-Dihydroxycholecalciferol
3,4-Dihydroxy-alpha-((isopropylamino)methyl)benzyl alcohol
1-Dihydroxyphenyl-1-alanine
1-(−)-3-(3,4-Dihydroxyphenyl)-2-methylanine
17R,21-alpha-Dihydroxy-4-propylajmalanium hydrogen tartrate
DI(2-Hydroxy-n-propyl) amine
Diisobutyl adipate
Diisobutyl phthalate
alpha-(2-(Diisopropylamino)ethyl)-alpha-phenyl-2-pyridineacetamide
Dilantin
Dilaudid
Diltiazem hydrochloride
Dimatif
Dimethoxy ethyl phthalate
1,2-Dimethoxyethane
3,6-Dimethoxy-4-sulfanilamidopyridazine
Dimethyl adipate
O,O-Dimethyl methylcarbamoylmethyl phosphordithioate
Dimethyl phthalate
Dimethyl sulfate
Dimethyl sulfoxide
O,S-Dimethyl phosphoramidothioate
N,N-Dimethylacetamide
O,O-Dimethyl-S-(2-(acetylamino)ethyl) dithiophosphate
4-(Dimethylamine)-3,5-XYLYL-N-methylcarbamate
Dimethylaminoantipyrine
4-Dimethylaminoazobenzene
para-Dimethylaminobenzenediazosodium sulphonate
5-(3-(Dimethylamino)propyl)-2-hydroxy-10,11-dihydro-5H-dibenz(b,f)azephine
11-(3-Dimethylaminopropylidene-6,11-dihydrodibenzo(b,e)thiepine hydrochloride
10-(2-(Dimethylamino)propyl)phenothiazine
Dimethylbenzanthracene
1,1-Dimethylbiguanide
1-(2-(1,3-Dimethyl-2-butenylidene)hydrazino)phthalazine
Dimethyldicetylammonium chloride
9,9-Dimethyl-10-dimethylaminopropylacridan hydrogen tartrate
6-alpha,21-Dimethylethisterone
N-(5-(((1,1-Dimethylethyl)amino)sulfonyl)-1,3,4-thiadiazol-2-YL)acetamide monsodium salt
N,N-Dimethyl-para((para-fluorophenyl)azo)aniline
Dimethylformamide
1,1-Dimethylhydrazine
1,2-Dimethylhydrazine
2,6-Dimethylhydroquinone
Dimethylimipramine
1,3-Dimethylisothiourea
1,3-Dimethylnitrosourea
3,3-Dimethyl-1-phenyltriazene
Dimethylthiomethylphosphate
N,N-Dimethyl-4-(para-tolylazo)aniline
5-(3,3-Dimethyl-1-triazeno)imidazole-4-carboxamide citrate
2,6-Dimethyl-4-tridecylmorpholine
1,3-Dimethylurea
2,4-Dinitroaniline
4,6-Dinitro-ortho-cresol ammonium salt
2,6-Dinitro-N,N-dipropyl-4-(trifluoromethyl)benzenamine
2,4-Dinitrophenol
2,4-Dinitrophenol sodium salt
Dinitrosopiperazine
2,4-Dinitrotoluene
2,6-Dinitrotoluene
Dinoprost methyl ester
Dinoprostone
n-Dioctyl phthalate
Dioxane
meta-Dioxane-4,4-dimethyl
1,4-Di-N-oxide of dihydroxymethylquinoxaline
1,3-Dioxolane-4-methanol
3-(2-(1,3-Dioxo-2-methylindanyl)) glutarimide
3-(2-(1,3-Dioxo-2-phenyl-4,5,6,7-tetrahydro-4,7-dithiaindanyl)) glutarimide
2-(2,6-Dioxopiperiden-3YL)phthalimide
N-(2,6-Dioxo-3-piperidyl)phthalimidine
1,3-Dioxo-2-(3-pyridylmethylene)indan
Diphenylamine
Diphenylguanidine
Diphenylhydantoin and phenobarbital
3-(3,3-Diphenylpropylamino)propyl-3′,4′,5′-trimethoxybenzoate hydrochloride
Dipropyl adipate
Diquat
DI-sec-octyl phthalate
Disodium ethylene-1,2-bisidithiocarbamate
Disodium etidronate
Disodium inosinate
Disodium methanearsenate
Disodium molybdate dihydrate
Disodium phosphonomycin
Disodium selenate
Disulfiram
Dithane M-45
2,2-Dithiobis(pyridine-1-oxide)magnesium sulfate trihydrate
2,2-Dithiodipyridine-1,1,-dioxide
Diuron
alpha-DFMO
Dobutamine hydrochloride
Domperidone
Dopamine
Dopamine hydrochloride
Doriden
Doxifluridine
Doxycycline
1-Dromoran tartrate
Duazomycin
Durabolin
Duricef
Dydrogesterone
Dye C
Econazole nitrate
Eflornithine hydrochloride
Elasiomycin
Elavil
Elavil hydrochloride
Elymoclavine
EM 255
Emoquil
Emorfazone
Enalapril maleate
Enavid
Endosulfan
Endrin
Enflurane
Enoxacin
Epe
Ephedrine
Epichlorohydrin
Epidehydrocholesterin
2-alpha,3-alpha-Epithio-5-alpha-androstan-17-beta-OL
4,5-Epithiovaleronitrile
EPN
Epocelin
1,2-Epoxyethylbenzene
Eraldin
Ergochrome AA (2,2)-5-beta,6-alpha,10-beta-5′,6′-alpha,1-,-beta
Ergocornine methanesulfonate (salt)
Ergotamine tartrate
Ergoterm TGO
Erythromycin
Escherichia coli endotoxin
Escin
beta-Escin
Escin, sodium salt
Estradiol
Estradiol dipropionate
Estradiol polyester with phosphoric acid
Estradiol-17-valerate
Estradiol-3-benzoate
Estradiol-3-benzoate mixed with progesterone (1:14 moles)
Estradiol-17-caprylate
Estramustin phosphate sodium
Estra-1,3,5(10)-triene-17-beta-diol-17-tetrahydropyranyl ether
Estriol
Estrone
Ethanolamine
Ethinamate
Ethinyl estradiol
Ethinyl estradiol and norethindrone acetate
17-alpha-Ethinyl-5,10-estrenolone
dl-Ethionine
Ethisterone and diethylstilbestrol
6-Ethoxy-2-benzothiazolesulfonamide
2-Ethoxyethanol
2-Ethoxyethyl acetate
Ethyl alcohol
Ethyl all-trans-9-(4-methoxy-2,3,6-trimethylphenyl)-3,7-dimethyl-2,4,6,8-nonatetraenoate
Ethyl apovincaminate
Ethyl benzene
Ethyl (2,4-dichlorophenoxy) acetate
Ethyl fluclozepate
Ethyl hexylene glycol
Ethyl mercury chloride
Ethyl methacrylate
Ethyl methanesulfonate
Ethyl methyl 1,4-dihydro-2,6-dimethyl-4-(meta-nitrophenyl)-3,5-pyridinedicarboxylate
Ethyl morphine hydrochloride dihydrate
Ethyl thiourea
alpha-((Ethylamino)methyl)-meta-hydroxybenzyl alcohol
2-Ethylamino-1,3,4-thiadiazole
1-Ethyl-1,4-dihydro-7-methyl-4-oxo-1,8-naphthyridine-3-carboxylic acid
Ethyl-S-dimethylaminoethyl methylphosphonothiolate
Ethyl-N,N-dimethyl carbamate
Ethylene bis(dithiocarbamato)) zinc
Ethylene chlorohydrin
1,2-Ethylene dibromide
Ethylene dichloride
Ethylene glycol
Ethylene glycol diethyl ether
Ethylene glycol methyl ether
Ethylene oxide
Ethylenebis (dithiocarbamato) manganese and zinc acetate (50:1)
Ethylenediamine hydrochloride
Ethylenediaminetetraacetic acid
Ethylenediaminetetraacetic acid, disodium salt
Ethyleneimine
Ethylestrenol
2-Ethylhexanol
Ethyl-para-hydroxyphenyl ketone
Ethylmercuric phosphate
Ethyl-N-methyl carbamate
Ethyl-2-methyl-4-chlorophenoxyacetate
5-Ethyl-N-methyl-5-phenylbarbituric acid
2-Ethyl-2-methylsuccinimide
1-Ethyl-4-(2-morpholinoethyl)-3,3-diphenyl-2-pyrrolidinone
N-Ethyl-N-nitrosobiuret
1-Ethyl-1-nitrosourea
Ethylnorgestrienone
17-Ethyl-19-nortestosterone
N-Ethyl-para-(phenylazo) aniline
5-Ethyl-5-phenylbarbituric acid
1-5-Ethyl-5-phenylhydantoin
3-Ethyl-5-phenylhydantoin
5-(2-Ethylphenyl)-3-(3-methoxyphenyl)-s-triazole
2-Ethylthioisonicotinamide
Ethyltrichlorphon
Ethyl-3,7,11-trimethyldodeca-2,4-dienoate
Ethylurea and sodium nitrite (1:1)
Ethylurea and sodium nitrite (2:1)
Ethynodiol
Ethynylestradiol mixed with norethindrone
2-alpha-Ethynyl-alpha-nor-17-alpha-pregn-20-YNE-2-beta,17-beta-diol
Etizolam
Etoperidone
ETP
E. typhosa lipopolysaccharide
False hellebore
Famfos
Famotidine
FD&C red No. 2
FD&C yellow NO. 5
Feldene
Fencahlonine
Fenestrel
Fenoprofen calcium dihydrate
Fenoterol hydrobromide
Fenthion
Fenthiuram
Ferbam
Ferrous sulfate
Fertodur
Fiboran
Firemaster BP-6
Firemaster FF-1
Flavoxate hydrochloride
Flomoxef sodium
Floxapen sodium
Flubendazole
Flucortolone
Flunarizine dihydrochloride
Flunisolide
Flunitrazepam
Fluoracizine
N-Fluoren-2-YL acetamide
Fluorobutyrophenone
Fluorocortisone
5-Fluoro-2,-deoxycytidine
3-Fluoro-4-dimethylaminoazobenzene
Fluorohydroxyandrostenedione
2-Fluoro-alpha-methyl-(1,1,-biphenyl)-4-acetic acid 1-(acetyloxy) ethyl ester
4,-Fluoro-4-(4-methylpiperidino)butyrophenone hydrochloride
3-Fluoro-4-phenylhydratropic acid
5-Fluoro-1-(tetrahydrofuran-2-YL)uracil
Fluorouracil
Flutamide
Flutazolam
Flutoprazepam
Flutropium bromide hydrate
Folic acid
Fominoben hydrochloride
Fonazine mesylate
Formaldehyde
Formamide
Formhydroxamic acid
Formoterol fumarate dihydrate
N-Formyl-N-hydroxyglycine
N-Formyljervine
Forphenicinol
Fortimicin A
Fortimicin A sulfate
Fotrin
Fulvine
Fumidil
Furapyrimidone
Furazosin hydrochloride
2-(2-Furyl)-3-(5-nitro-2-furyl)acrylamide
Fusarenone X
Fusaric acid calcium salt
Fusariotoxin T 2
Fusidine
Fyrol FR 2
Gabexate mesylate
Galactose
Gastrozepin
Gentamycin
Gentamycin sulfate
Gentisic acid
Germanium dioxide
Gestoral
Gindarine hydrochloride
Glucagon
2-(beta-d-Glucopyranosyloxy) isobutyronitrile
d-Glucose
Gludiase
Glutaraldehyde
Glutril
Glycidol
Glycinonitrile
Glycinonitrile hydrochloride
Glycol ethers
Glycyrrhizic acid, ammonium salt
Gold sodium thiomalate
Gonadotropin releasing hormone agonist
Gossypol acetic acid
Grisofulvin
Guanabenz acetate
Guanazodine
Guanfacine hydrochloride
Guanine-3-N-oxide
Guanosine
HBK
Haloanisone
Halofantrine hydrochloride
Haloperidol decanoate
Halopredone acetate
Halothane
Haloxazolam
HCDD
Heliotrine
Hematoidin
Heptamethylphenylcyclotetrasiloxane
Heptyl phthalate
Heroin
Hexabromonaphthalene
Hexachlorobenzene
2,2′,4,4′,5′5′-Hexachloro-1,1,-biphenyl
3,3′,4,4′,5,5′-Hexachlorobiphenyl
Hexachlorobutadiene
Hexachlorocyclopentadiene
1,2,3,4,7,8-Hexachlorodibenzofuran
Hexachlorophene
4,5,6,7,8,8-Hexachlor-D1,5-tetrahydro-4,7-methanoinden
1-Hexadecanamine
Hexadecyltrimethylammonium bromide
Hexafluoroacetone
Hexafluoro acetone trihydrate
Hexamethonium bromide
Hexamethylmelamine
n-Hexane
1,6-Hexanediamine
2-Hexanone
Hexocyclium methylsulfate
Hexone
Hexoprenaline dihydrochloride
Hexoprenaline sulfate
n-Hexyl carborane
Histamethizine
Histamine diphosphate
Homofolate
Human immunoglobin COG-78
Hyaluronic acid, sodium salt
Hycanthone methanesulfonate
Hydantoin
Hydralazine
Hydralazine hydrochloride
Hydrazine
Hydrochlorbenzethylamine dimaleate
Hydrochloric acid
Hydrocortisone sodium succinate
Hydrocortisone-21-acetate
Hydrocortisone-17-butyrate
Hydrocortisone-17-butyrate-21-propionate
Hydrocortisone-21-phosphate
Hydrofluoric acid
10-beta-Hydroperoxy-17-alpha-ethynyl-4-estren-17-beta-OL-3-one
Hydroquinone-beta-d-glucopyranoside
N-Hydroxy ethyl carbamate
4,-Hydroxyacetanilide
N-Hydroxy-N-acetyl-2-aminofluorene
N-Hydroxyadenine
6-N-Hydroxyadenosine
3-alpha-Hydroxy-17-androston--one
17-beta-Hydroxy-5-beta-androstan-3-one
3-Hydroxybenzoic acid
para-Hydroxybenzoic acid ethyl ester
5-(alpha-Hydroxybenzyl)-2-benzimidazolecarbamic acid methyl ester
1-Hydroxycholecalciferol
Hydroxydimethylarsine oxide
Hydroxydimethylarsine oxide, sodium salt
9-Hydroxyellipticine
2-(2-Hydroxyethoxy)ethyl-N-(alpha,alpha,alpha-trifluoro-meta-tolyl)anthranilate
Hydroxyethyl starch
beta-Hydroxyethylcarbamate
1-Hydroxyethylidene-1,1-diphosphonic acid
17-beta-Hydroxy-7-alpha-methylandrost-5-ENE-3-one
7-Hydroxymethyl-12-methylbenz(alpha)anthracene
1-Hydroxymethyl-2-methylditmide-2-oxide
5-Hydroxymethyl-4-methyluracil
2-Hydroxymethylphenol
5-(1-Hydroxy-2-((1-methyl-3-phenylpropyl)amino)ethyl) salicyclamide hydrochloride
N-(Hydroxymethyl)phthalimide
3-(1-Hydroxy-2-piperidinoethyl)-5-phenylisoxazole citrate
2-Hydroxy-N-(3-(meta-(piperidinomethyl)phenoxy)propyl)acetamide acetate (ester hydrochloride)
Hydroxyprogesterone caproate
beta-(N-(3-Hydroxy-4-pyridone))-alpha-aminopropionic acid
4-Hydroxysalicylic acid
5-Hydroxytetracycline
5-Hydroxytetracycline hydrochloride
17-beta-Hydroxy-4,4,17-alpha-trimethyl-androst-5-ENE(2,3-d) isoxazole
Hydroxytriphenylstannane
dl-Hydroxytryptophan
5-Hydroxy-1-tryptophan
dl-Hydroxytryptophan
5-Hydroxy-1-tryptophan
Hydroxyurea
3-Hydroxyxanthine
Hydroxyzine pamoate
Hyoscine hydrobromide
Hypochlorous acid
Hypoglycine B
Ibuprofen piconol
Ifenprodil tartrate
IMET 3106
4-Imidazo (1,2-alpha) pyridin-2-YL-alpha-methylbenzeneacetic acid
Imidazole mustard
2-Imidazolidinethione
2-Imidazolidinethione mixed with sodium nitrite
2-Imino-5-phenyl-4-oxazolidinone
Improsulfan tosylate
Indacrinone
Indanazoline hydrochloride
1,3-Indandione
Indapamide
Indeloxazine hydrochloride
Inderal
Indium
Indium nitrate
1H-Indole-3-acetic acid
Indole-3-carbinol
Indomethacin
Inolin
Insulin
Insulin protamine zinc
Iocarmate meglumine
Iodoacetic acid
Iopramine hydrochloride
Iotroxate meglumine
Ipratropium bromide
Iron-dextran complex
Iron nickel zinc oxide
Iron-poly (sorbitol-gluconic acid) complex
Iron-sorbitol
Isoamygdalin
Isoamyl 5,6-dihydro-7,8-dimethyl-4,5-dioxo-4H-pyrano (3,2-c) quinoline-2-carboxylate
Isobutyl methacrylate
para-Isobutylhydratropic acid
Isocarboxazid
Isodecyl methacrylate
Isodonazole nitrate
Isoflurane
Isonicotinic acid hydrazide
Isonicotinic acid-2-isopropylhydrazide
Isooctyl-2,4-dichlorophenoxyacetate
Isophosphamide
Isoprenaline hydrochloride
Isoprenyl chalcone
Isopropyl alcohol
Isopropyl-2,4-D ester
Isopropylidine azastreptonigrin
4,4,-Isopropylidenediphenol, polymer with 1-chloro-2,3-epoxypropane
Isopropylmethanesulfonate
Isosafrole-n-octylsulfoxide
Isothiacyanic acid, ethylene ester
Isothiocyanic acid, phenyl ester
Isothiourea
Jervine
Jervine-3-acetate
Josamycin
Kanamycin
Kanamycin sulfate (1:1) salt
KAO 264
Karminomycin
Kepone
Kerlone
Ketamine
Ketoprofen sodium
Ketotifen fumarate
KF-868
Khat leaf extract
KM-1146
KPE
Lactose
Latamoxef sodium
Lead
Lead (II) acetate
Lead chloride
Lead (II) nitrate (1:2)
Lecithin iodide
Lenampicillin hydrochloride
Lendormin
Lente insulin
Lentinan
Leptophos
1-Leucine
Leurocristine
Leurocristine sulfate (1:1)
Levamisole hydrochloride
Levorin
Levothyroxine sodium
Librium
d-Limonene
Linear alkylbenzenesulfonate, sodium salt
Linoleic acid (oxidized)
Liothyronine
Lipopolysaccharide, escherichia coli
Lipopolysaccharide, from B. Abortus Bang.
Lithium carbonate (2:1)
Lithium carmine
Lithium chloride
Lividomycin
Lobenzarit disodium
Locoweed
Lofetensin hydrochloride
Lucanthone metabolite
Luteinizing hormone antiserum
Luteinizing hormone-releasing hormone
Luteinizing hormone-releasing hormone, diacetate (salt)
Luteinizing hormone-releasing hormone, diacetate, tetrahydrate
Lyndiol
Lysenyl hydrogen maleate
d-Lysergic acid diethylamide tartrate
Lysergide tartrate
Lysine
Mafenide acetate
Magnesium glutamate hydrobromide
Magnesium sulfate (1:1)
Malathion
Maleimide
Malotilate
Maltose
Manganese (II) chloride
Manganese (II) ethylenebis (dithiocarbamate)
Manganese (II) sulfate (1:1)
Maprotiline hydrochloride
Marezine hydrochloride
Maytansine
Mazindol
Mec
Meclizine dihydrochloride
Meclizine hydrochloride
Medemycin
Medrogestone
Medroxyprogesterone
Medroxyprogesterone acetate
Medullin
Melengestrol acetate
Mentha arvensis, oil
Mepiprazole dihydrochloride
Mepyrapone
Mequitazine
2-Mercapto-1-methylimidazole
1-(d-3-Mercapto-2-methyl-1-oxopropyl)-1-proline (S,S)
N-(2-Mercapto-2-methylpropanoyl)-1-cysteine
6-Mercaptopurine monohydrate
6-Mercaptopurine 3-N-oxide
Mercaptopurine ribonucleoside
d,3-Mercaptovaline
Mercuric acetate
Mercuric oxide
Mercury
Mercury (II) chloride
Mercury (II) iodide
Mercury methylchloride
Merthiolate sodium
Mervan ethanolamine salt
Mescaline
Mesoxalylurea monohydrate
Mestranol mixed with norethindrone
Metalutin
Metaproterenol sulfate
Methadone
Methadone hydrochloride
dl-Methadone hydrochloride
Methallyl-19-nortestosterone
Methaminodiazepoxide hydrochloride
1-Methamphetamine hydrochloride
Methaqualone hydrochloride
Methedrine
dl-Methionine
1-Methionine
Methionine sulfoximine
Methofadin
Methophenazine difumarate
Methotrexate
Methotrexate sodium
Methoxyacetic acid
3-Methoxycarbonylaminophenyl-N-3,-methylphenylcarbamate
Methoxychlor
5-Methoxyindoleacetic acid
4-(6-Methoxy-2-naphthyl)-2-butanone
(+)-2-(Methoxy-2-naphthyl)-propionic acid
2-(3-Methoxyphenyl)-5,6-dihydro-s-triazolo (5,1-alpha) isoquinoline
2-(para-(6-Methoxy-2-phenyl-3-indenyl)phenoxy)triethylamine hydrochloride
2-(para-(para-Methoxy-alpha-phenylphenethyl)phenoxy)triethylamine hydrochloride
N1-(3-Methoxy-2-pyrazinyl)sulfanilamide
Methyl alcohol
Methyl azoxymethyl acetate
Methyl benzimidazole-2-YL carbamate
2-Methyl butylacrylate
Methyl chloride
Methyl chloroform
Methyl (beta)-11-alpha-16-dihydroxy-16-methyl-9-oxoprost-13-EN-1-OATE
Methyl ethyl ketone
Methyl hydrazine
Methyl isocyanate
Methyl mesylate
Methyl methacrylate
Methyl (methylthio) mercury
Methyl parathion
Methyl pentachlorophenate
Methyl phenidyl acetate
Methyl salicylate
Methyl thiourea
Methyl urea and sodium nitrite
Methylacetamide
Methyl-5-benzoyl benzimidazole-2-carbamate
1-Methyl-2-benzylhydrazine
1-Methyl-5-chloroindoline methylbromide
Methylchlortetracycline
3-Methylcholanthrene
N-Methyl-4-cyclochexene-1,2-dicarboximide
N-Methyl-N-desacetylcolchicine
N-Methyl-dibromomaleinimide
beta-Methyldigoxin
17-alpha-Methyldihydrotestosterone
N-Methyl-3,6-dithia-3,4,5,6-tetrahydrophthalimide
Methylene chloride
Methylene dimethanesulfonate
N,N,-Methylenebis(2-amino-1,3,4-thiadiazole)
2-Methylenecyclopropanylalanine
Methylergonovine maleate
3-(1-Methylethyl)-1H-2,1,3-benzothiazain-4(3H)-one-2,2-dioxide
4-Methylethylenethiourea
3-Methyl-5-ethyl-5-phenylhydantoin
3-Methylethynylestradiol
x-Methylfolic acid
N-Methylformamide
Methylhesperidin
(alpha-(2-Methylhydrazino)-para-toluoyl)urea, monohydrobromide
4-Methyl-7-hydroxycoumarin
Methyl-ortho-(4-hydroxy-3-methoxycinnamoyl) reserpate
2-Methyl-1,3-indandione
N-Methyljervine
N-Methyllorazepam
Methylmercuric dicyandiamide
Methylmercuric phosphate
Methylmercury
Methylmercury hydroxide
1-Methyl-6-(1-methylallyl)-2,5-dithiobiurea
d-3-Methyl-N-methylmorphinan phosphate
N-Methyl-alpha-methyl-alpha-phenylsuccinimide
2-Methyl-1,4-naphthoquinone
2-Methyl-5-nitroimidazole-1-ethanol
N-Methyl-N′-nitro-N-nitrosoguanidine
4-(N-Methyl-N-nitrosamino)-1-(3-pyridyl)-1-butanone
N-Methyl-N-nitrosoaniline
N-Methyl-N-nitrosoethylcarbamate
N-Methyl-N-nitroso-1-propanamine
N-Methyl-N-nitrosourea
(3-Methyl-4-oxo-5-piperidino-2-thiazolidinylidene) acetic acid ethyl ester
10-Methylphenothiazine-2-acetic acid
N-Methyl-para-(phenylazo) aniline
3-Methyl-2-phenylmorpholine hydrochloride
N-Methyl-2-phenyl-succinimide
Methyl-4-phthalimido-dl-glutaramate
N-Methyl-2-phthalimidoglutarimide
N-Methylpyrrolidone
Methylsulfonyl chloramphenicol
17-Methyltestosterone
N-Methyl-3,4,5,6-tetrahydrophthalimide
Methylthioinosine
6-Methylthiouracil
6-Methyluracil
Metiapine
Meticrane
Metoprine
Metoprolol tartrate
Metrizamide
Mexiletine hydrochloride
Mezinium methyl sulfate
Mezlocillin
Mibolerone
Miconazole nitrate
Micromycin
Midodrine
Mikelan
Miloxacin
Miltown
Mineral oil
Mineral oil, petroleum extracts, heavy naphthenic distillate solvent
Mirex
Mithramycin
MN-1695
Mobilat
Molybdenum
Monoethylhexyl phthalate
Monoethylphenyltriazene
8-Monohydro mirex
Monosodium glutamate
Morphine hydrochloride
Morphine sulfate
Morphocycline
Moxestrol
Moxnidazole
Mucopolysaccharide, polysulfuric acid ester
Muldamine
Mycosporin
Nafoxidine hydrochloride
Naftidrofuryl oxalate
Naja nigricollis venom
Naloxone hydrochloride
Naphthalene
beta-Naphthoflavone
1-Naphthol
Navaron
Neem oil
Nembutal sodium
Neocarzinostatin
Neoprene
Neoproserine
Neosynephrine
Netilmicin sulfate
Nickel
Nickel carbonyl
Nickel compounds
Nickel subsulfide
Nickelous chloride
Nicotergoline
Nicotine
Nicotine tartrate (1:2)
N-Nicotinoyltryptamide
Nipradilol
Nisentil
Nitric acid
Nitrilotriacetic acid trisodium salt monohydrate
Nitrobenzene
Nitrofurantoin
Nitrofurazone
4-((5-Nitrofurfurylidene)amino)-3-methylthiomorpholine-1,1-dioxide
Nitrogen dioxide
Nitrogen oxide
Nitroglycerin
1-(2-Nitroimidazol-1-YL-3-methoxypropan-2-OL
Nitromifene citrate
2-Nitropropane
4-Nitroquinoline-N-oxide
Nitroso compounds
N-Nitroso compounds
N-Nitrosobis(2-oxopropyl)amine
Nitrosocimetidine
N-Nitrosodiethylamine
N-Nitrosodimethylamine
N-Nitrosodi-N-propylamine
N-Nitroso-N-ethyl aniline
N-Nitroso-N-ethylurethan
N-Nitroso-N-ethylvinylamine
N-Nitrosohexahydroazepine
N-Nitrosoimidazolidinethione
N-Nitrosopiperidine
1-(Nitrosopropylamino)-2-propanol
N-Nitroso-N-propylurea
Nizofenone fumarate
Norchlorcyclizine
Norchlorcyclizine hydrochloride
1-Norepinephrine
19-Norethisterone
Norethisterone enanthate
Norgestrel
1-Norgestrel
19-Norpregn-4-ENE-3,20-dione
19-Nor-17-alpha-pregn-5(10)-EN-20-YNE-3-alpha,17-diol
19-Nor-17-alpha-pregn-5(10)-EN-20-YNE-3-beta,17-diol
19-Nor-17-alpha-pregn-4-EN-20-YN-17-OL
Novadex
Nutmeg oil, east indian
Nystatin
Ochratoxin
Ochratoxin A sodium salt
Octabromodiphenyl
Octachlorodibenzodioxin
Octoclothepine
Ofloxacin
Oleamine
Oleylamine hydrofluoride
Oncodazole
Ophthazin
Orgoteins
Orphenadrine hydrochloride
Oxaprozin
Oxatimide
Oxazolazepam
Oxepinac
Oxfendazole
Oxibendazole
Oxiranecarboxylic acid, 3-(((3-methyl-1-(((3-methylbutyl)amino) carbonyl)-,ethyl ester, (2S-(2-alpha-3-beta)R*)))
N-(2-Oxo-3,5,7-cylcoheptatrien-1-YL)aminooxoacetic acid ethyl ester
2-(3-Oxo-1-indanylidene)-1,3-indandione
Oxolamine citrate
N-(2-Oxo-3-piperidyl)phthalimide
Oxybutynin chloride
Oxymorphinone hydrochloride
beta-Oxypropylpropylnitrosamine
Ozone
Padrin
Palm oil
Panoral
d-Pantethine
Pantocrin
Papain
Papaverine chlorohydrate
Paradione
Paramathasone acetate
Paraquat dichloride
Parathion
Paraxanthine
Pavisoid
PE-043
Penfluridol
Penicillic acid
Penitrem A
Pentachlorobenzene
2,3,4,7,8-Pentachlorodibenzofuran
Pentachloronitrobenzene
Pentachlorophenol
Pentafluorophenyl chloride
Pentazocine hydrochloride
Pentostatin
Pentothal
Pentothal sodium
Pentoxyphylline
Perchloroethylene
Perdipine
Perfluorodecanoic acid
Periactin hydrochloride
Periactinol
Perphenazine hydrochloride
Pharmagel A
1,10-Phenanthroline
Phenazin-5-oxide
Phenethyl alcohol
Phenfluoramine hydrochloride
Phenol
4-Phenoxy-3-(pyrrolidinyl)-5-sulfamoylbenzoic acid
Phenyl salicylate
Phenylacetic acid
(Phenylacetyl) urea
1-Phenylalanine
17-beta-Phenylaminocarbonyloxyoestra-1,3,5(10)-triene-3-methyl ether
para-(Phenylazo)aniline
2-Phenyl-5-benzothiazoleacetic acid
1-Phenyl-3,3-diethyltriazene
2-Phenyl-5,5-dimethyl-tetrahydro-1,4-oxazine hydrochloride
1-Phenyl-2-(1′,1′-diphenylpropyl-3′-amino)propane
4-Phenyl-1,2-diphenyl-3,5-pyrazolidinedione
meta-Phenylenediamine
2-Phenylethylhydrazine
Phenylmethylcylosiloxane, mixed copolymer
N-Phenylphthalimidine
Phenyl-2-pyridylmethyl-beta-N,N-dimethylaminoethyl ether succinate
2-(Phenylsulfonylamino)-1,3,4-thiadiazole-5-sulfonamide
1-Phenyl-2-thiourea
Phomopsin
Phorbol myristate acetate
Phosphonacetyl-1-aspartic acid
Phosphoramide mustard cyclohexylamine salt
Phthalazinol
Phthalic anhydride
Phthalimide
Phthalimidomethyl-O,O-dimethyl phosphorodithioate
N-Phthaloly-1-aspartic acid
N-Phthalylisoglutamine
Physostigmine sulfate
Phytohemagglutinin
Picloram
Pilocarpine monohydrochloride
Pimozide
2,6-Piperazinedione-4,4,-propylene dioxopiperazine
Piperidine
3-Piperidine-1,1-diphenyl-propanol-(1) methanesulphonate
Piperin
Piperonyl butoxide
Pipethanate ethylbromide
Pipram
Pituitary growth hormone
Plafibride
cis-Platinous diammine dichloride
Platinum thymine blue
Podophyllin
Podophyllotoxin
Polybrominated biphenyls
Polychlorinated biphenyl (Aroclor 1248)
Polychlorinated biphenyl (Aroclor 1254)
Polychlorinated biphenyl (Kanechlor 300)
Polychlorinated biphenyl (Kanechlor 400)
Polychlorinated biphenyl (Kanechlor 500)
Polyoxyethylene sorbitan monolaurate
Potassium bichromate
Potassium canrenoate
Potassium chromate (VI)
Potassium clavulanate
Potassium cyanide
Potassium fluoride
Potassium iodide
Potassium nitrate
Potassium nitrite (1:1)
Potassium perchlorate
Potassium thiocyanate
Potato blossoms, glycoalkaloid extract
Potato, green parts
Pranoprofen
Prednisolone succinate
Prednisone 21-acetate
Predonin
9-beta,10-alpha-Pregna-4,6-diene-3,20-dione and 17-alpha-hydroxypregn-4-ENE-3,2 ortho-dione (9:10)
5-alpha-17-alpha-Pregna-2-EN-20-YN-17-OL, acetate
Premarin
Primaquine phosphate
Primobolan
Prinadol hydrobromide
Procarbazine
Procarbazine hydrochloride
Procaterol hydrochloride
Prochlorpromazine
Progesterone
Prolinomethyltetracycline
Promethazine hydrochloride
Propadrine hydrochloride
Propane sultone
1,3-Propanediamine
1,2-Propanediol
Propanidide
3-Propanolamine
Proparthrin
Propazone
Propiononitrile
Propoxur
2-Propoxyethyl acetate
d-Propoxyphene hydrochloride
Propyl carbamate
Propyl cellosolve
n-Propyl gallate
Propylene glycol diacetate
Propylene glycol monomethyl ether
Propylene oxide
2-Propylpentanoic acid
2-Propylpiperidine
6-Propyl-2-thiouracil
Propylthiouracil and iodine
2-Propylvaleramide
2-Propylvaleric acid sodium salt
Prostaglandin A1
Prostaglandin E1
Prostaglandin E2 sodium salt
Prostaglandin F1-alpha
Prostaglandin F2-alpha
Prostaglandin F2-alpha-tham
Protizinic acid
Proxil
Pseudolaric acid A
Pseudolaric acid B
Purapuridine
Purine-6-thiol
Pyrantel pamoate
Pyrazine-2,3-dicarboxylic acid imide
Pyrazole
Pyrbuterol hydrochloride
Pyridinamine (9CI)
2,3-Pyridinedicarboximide
3,4-Pyridinedicarboximide
1-(Pyridyl-3)-3,3-dimethyl triazene
1-Pyridyl-3-methyl-3-ethyltriazene
5-(para-(2-Pyridylsulfamoyl)phenylazo)salicyclic acid
Pyrimidine-4,5-dicarboxylic acid imide
N1-2-Pyrimidinyl-sulfanilamide
Pyrogallol
Pyronaridine
N-(1-Pyrrolidinylmethyl)-tetracycline
Quaalude
Quercetin
Quinine
2-Quinoline thioacetamide hydrochloride
Ralgro
Refosporen
Reptilase
Reserpine
Retinoid etretin
all-trans-Retinylidene methyl nitrone
Rhodamine 6G extra base
2-beta-d-Ribofuranosyl-as-triazine-3,5(2H,4H)-dione
1-beta-d-Ribofuranosyl-1,2,4-triazole-3-carboxamide
Ricin
Rifamycin AMP
Rifamycin SV
Ripcord
Ritodrine hydrochloride
Rizaben
Robaveron
Ronnel
Rose bengal sodium
Rotenone
Rowachol
Rowatin
R Salt
Rubratoxin B
Rythmodan
Salicyclaldehyde
Salicyclamide
Salicyclic acid
Salicyclic acid, compounded with morpholine (1:1)
ortho-Salicylsalicylic acid
Salipran
Salmonella enteritidis endotoxin
Sarkomycin
SCH 20569
Scopolamine
Sefril
Selenium
Selenodiglutathione
Semicarbazide hydrochloride
Serum gonadotropin
Sfericase
Silicone 360
Sisomicin
S. Marcescens lipopolysaccharide
Smoke condensate, cigarette
Smokeless tobacco
Sodium para-aminosalicylate
Sodium arsenite
Sodium benzoate
Sodium bicarbonate
Sodium chloride
Sodium chlorite
Sodium chondroitin polysulfate
Sodium cobaltinitrite
Sodium colistinemethanesulfonate
Sodium cyanide
Sodium cyclamate
Sodium dehydroacetic acid
Sodium dichlorocyanurate
Sodium diethyldithiocarbamate
Sodium diphenyldiazo-bis(alpha-naphthylaminesulfonate)
Sodium fluoride
Sodium (E)-3-(para-(1H-imidazol-1-methyl)phenyl)-2-propenoate
Sodium iodide
Sodium lauryl sulfate
Sodium luminal
Sodium nigericin
Sodium nitrite
Sodium nitrite and carbendazime (1:1)
Sodium nitrite and 1-citrulline (1:2)
Sodium nitrite and 1-(methylethyl) urea
Sodium nitroferricyanide
Sodium pentachlorophenate
Sodium picosulfate
Sodium piperacillin
Sodium retinoate
Sodium saccharin
Sodium salicylate
Sodium selenite
Sodium selenite pentahydrate
Sodium sulfate (2:1)
Sodium d-thyroxine
Sodium tolmetin dihydrate
Sodium-2,4-dichlorophenoxyacetate
(22s,25r)-5-alpha-Solanidan-3-beta-OL
Solanid-5-ENE-3-beta, 12-alpha-diol
(22s,25r)-Solanid-5-EN-3-beta-OL
Solanine
Solcoseryl
Spectogard
Spiclomazine hydrochloride
Spiramycin
Spiroperidol
SRC-II, heavy distillate
1-ST-2121
Sterculia foetida oil
Steroids
Stimulexin
Streptomycin
Streptomycin and dihydrostreptomycin
Streptomycin sesquisulfate
Streptomycin sulphate
Streptonigran
Streptonigrin methyl ester
Streptozoticin
STS 557
Styrene
Subtigen
Succinic anhydride
Succinonitrile
Sucrose
Sulfadiazine silver salt
Sulfadimethoxypyrimidine
Sulfadimethyldiazine
Sulfamonomethoxin
Sulfamoxole-trimethoprim mixture
Sulfanilamide
6-Sulfanilamido-2,4-dimethoxypyrimidine
5-Sulfanilamido-3,4-dimethyl-isoxazole
Sulfanilylurea
N-Sulfanylacetamide
alpha-Sulfobenzylpenicillin disodium
Sulfur dioxide
Sulfuric acid
Suloctidyl
Sultopride hydrochloride
Supercortyl
Superprednol
Surgam
Surital sodium
Surmontil maleate
Suxibuzone
Sweet pea seeds
Sygethin
meta-Synephrine hydrochloride
Synephrine tartrate
Synsac
2,4,5-T
T-1982
T-2588
Tagamet
Tarweed
TCDD
Tellurium
Tellurium dioxide
Temephos
Tenormin
Terbutaline sulphate
Terodiline hydrochloride
Testosterone
Testosterone heptanoate
Testosterone propionate
1,1,3,3-Tetrabutylurea
2,3,7,8-Tetrachlododibenzofuran
Tetrachloroacetone
1,1,3,3-Tetrachloroacetone
3,3′,4,4′-Tetrachloroazoxbenzene
1,2,3,4-Tetrachlorobenzene
3,3′,4,4′-Tetrachlorobiphenyl
2,4,5,6-Tetrachlorophenol
Tetracycline
Tetracycline hydrochloride
Tetraethyl lead
1-trans-D9-Tetrahydrocannabinol
2-(para-(1,2,3,4-Tetrahydro-2-(para-chlorophenyl)naphthyl) phenoxy) triethyl amine
2,3,4,5-Tetrahydro-2,8-dimethyl-5-(2-(6-methyl-3-pyridyl)ethyl)-1H-pyrid 0-(4,3-beta) indole
Tetrahydro-3,5-dimethyl-4H,1,3,5-oxadiazine-4-thione
5,6,7,8-Tetrahydrofolic acid
2-(1,2,3,4-Tetrahydro-1-naphthylamino)-2-imidazoline hydrochloride
4,-O-Tetrahydropyranyladriamycin hydrochloride
para-(1,1,3,3-Tetramethylbutyl)phenol, polymer with ethylene oxide and formaldehyde
2,2,9,9-Tetramethyl-1,10-decanediol
Tetramethyl lead
Tetramethylsuccinonitrile
Tetramethylthiourea
1,1,3,3-Tetramethylurea
Tetranicotylfructose
Tetrapotassium hexacyanoferrate
Tetrasodium fosfestrol
Tetrazosin hydrochloride dihydrate
Thalidomide
Thallium acetate
Thallium chloride
Thallium compounds
Thallium sulfate
Thebaine hydrochloride
para-(2-Thenoyl) hydratropic acid
Theobromine
Theobromine sodium salicylate
Theophylline
1-(Theophyllin-7-YL)ethyl-2-(2-(para-chlorophenoxy)-2-methylpropionate
Thiamine chloride
2-(Thiazol-4-YL) benzimidazole
2-(4-Thiazolyl)-5-benzimidazolecarbamic acid methyl ester
Thioacetamide
Thioinosine
Thiotriethylenephosphoramide
2-Thiouracil
Thiram
Thymidine
Thyroid
1-Thyroxin
Thyroxine
Tiapride hydrochloride
Ticarcillin sodium
Ticlodone
Timepidium bromide
Timiperone
Tinactin
Tindurin
Tinidazole
Tinoridine hydrochloride
Tiquizium bromide
2,4,5-T isooctyl ester
Titanium (wet powder)
Tizanidine hydrochloride
Tobacco
Tobacco leaf, nicotiana glauca
Tobramycin
Todralazine hydrochloride hydrate
Togal
Tolmetine
Toluene
para-Toluenediamine sulfate
ortho-Toluidine
Tormosyl
2,4,5-T propylene glycol butyl ether ester
Traxanox sodium pentahydrate
Triaminoguanidine nitrate
para,para,-Triazenylenedibenzenesulfonamide
Triazolam
Trichloroacetonitrile
1,2,4-Trichlorobenzene
Trichloroethylene
2,4,4,-Trichloro-2,-hydroxydiphenyl ether
(2,2,2-Trichloro-1-hydroxyethyl) dimethylphosphonate
N-(Trichloromethylthio)phthalimide
4-(2,4,5-Trichlorophenoxy) butyric acid
alpha-(2,4,5-Trichlorophenoxy) propionic acid
Trichloropropionitrile
Triclopyr
Tricosanthin
Tridemorph
Tridiphane
Triethyl lead chloride
Triethylenetetramine
2,2,2-Trifluoroethyl vinyl ether
3,-Trifluoromethyl-4-dimethylaminoazobenzene
Trifluoromethylperazine
2-(8,-Trifluoromethyl-4,-quinolylamino)benzoic acid, 2,3-dihydroxy propyl ester
Trifluperidol
Triglyme
Trimebutine maleate
(beta)-Trimethoquinol
Trimethoxazine
5-(3,4,5-Trimethoxybenzyl)-2,4-diaminopyrimidine
Trimethyl lead chloride
Trimethyl phosphate
Trimethyl phosphite
3,3,5-Trimethyl-2,4-diketooxazolidine
Trimethylenedimethanesulfonate
exo-Trimethylenenorbornane
1,1,3-Trimethyl-3-nitrosourea
1,3,5-Trimethyl-2,4,6-tris(3,5-DI-tert-butyl-4-hydroxybenzyl) benzene
Triparanol
Tris
Tris (1-aziridinyl)-para-benzoquinone
Tris-(1-aziridinyl) phosphine oxide
Trisaziridinyltriazine
Tris (1-methylethylene) phosphoric triamide
Tritolyl phosphate
Tropacaine hydrochloride
1-Tryptophan
TSH-releasing hormone
Tungsten
dl-meta-Tyrosine
1-Tyrosine
Ubiquinone 10
Uracil
Uracil mixture with tegafur (4:1)
Uranyl acetate dihydrate
Urapidil
Urbacide
Urbason soluble
Urethane
Urfamicin hydrochloride
Uridion
Urokinase
Valbazen
Valison
Vanadium pentoxide (dust)
Vasodilan
Vasodilian
Vasodistal
Vasotonin
Venacil
Ventipulmin
Veratramine
Veratrine
Veratrylamine
Vincaleukoblastine
Vincaleukoblastine sulfate (1:1) (salt)
Vinyl chloride
Vinyl pivalate
Vinyl toluene
Vinylidene chloride
R-5-Vinyl-2-oxazolidinethione
Viomycin
Vipera berus venom
Viriditoxin
Visken
Vistaril hydrochloride
Vitamin A
Vitamin A acetate
Vitamin A acid
13-cis-Vitamin A acid
Vitamin A palmitate
Vitamin B7
Vitamin B12 complex
Vitamin B12, methyl
Vitamin D2
Vitamin K
Vitamin MK 4
Volidan
Vomitoxin
Wait's green mountain antihistamine
Warfarin
Warfarin sodium
White spirit
Xamoterolfumarate
Xanax
Xanthinol nicotinate
Xylene
meta-Xylene
ortho-Xylene
para-Xylene
Xylostatin
N-(2,3-Xylyl)anthranilic acid
Ytterbium chloride
Zaroxolyn
Zearalenone
Zimelidine dihydrochloride
Zinc carbonate (1:1)
Zinc chloride
Zinc (II) EbrA complex
Zinc oxide
Zinc (N,N,-propylene-1,2-bis(dithiocarbamate))
Zinc pyridine-2-thiol-1-oxide
Zinc sulfate
Zoapatle, crude leaf extract
Zoapatle, semi-purified leaf extract
Zotepine
Zygosporin A
Zyloprim

TABLE V
Antibodies Used to Determine
the Differentiated Status of Cells

| | | |
|---|---|---|
| Antibody | Antigen | Cell Specificity |

Panel I: Undifferentiated Cells

| | |
|---|---|
| SSEA-1 | human ES/ICM |
| SSEA-4 | human ES/ICM |
| TRA-1-60 | human ES/ICM |
| TRA-1-81 | human ES/ICM |
| SOX-2 | human ES/ICM |
| Oct-4 | human ES/ICM |
| Nanog | human ES/ICM |

Panel II: Broad Differentiated Cell Characterization

| | |
|---|---|
| Cxcr4 | Definitive endoderm |
| Vimentin | Connective tissue cell./ |
| | primitive neuroepithelium |
| Cytokeratins | Epithelial cell |
| Neurofilaments | Neurons |
| L, M, H | |

Panel III: Narrow Differentiated Cell Characterization

| | | |
|---|---|---|
| Ectoderm | Nestin | Neural progenitor |
| | S-100 | Neuroectoderm |
| | CD56 | Neuroectoderm |
| | CD57 | Neuroectoderm |
| | CD99 | Neuroectoderm |
| | Neuron- | Neuroectoderm |
| | specific | |
| | enolase | |
| | Microtubule | Dendritic neurons |
| | Basic Protein | |
| | (MAP 2) | |
| | GFAP | Astrocytes |
| | CD133 | Neural stem cells |
| | Myelin basic | Oligodendrocytes |
| | Protein | |
| | Neural | Differentiated neurons |
| | Tubulin | |
| | Noggin | Neurons |
| | | Mesoderm |
| | Bone | Mesenchymal Progenitors |
| | morphogenic | |
| | protein receptor | |
| | Fetal liver | Endothelial progenitor |
| | kinase-1 (Flk1) | |
| | Smooth muscle | Smooth muscle |
| | myosin | |
| | VE-Cadherin | Smooth muscle |
| | Desmin | Muscle cell (multinucleate) |
| | Bone-specific | Osteoblast |
| | alkaline phosphatase | |
| | Osteocalcin | Osteoblast |
| | CD34 | Hematopoietic/muscle |
| | | satellite/Endothelial |
| | CD44 | Mesenchymal progenitors |
| | c-kit | Hematopoietic and |
| | | mesenchymal progenitors |
| | Stem cell | Hematopoietic/ |
| | antigen-1 | mesenchymal |
| | (sca-1) | |
| | Stro-1 | Bone marrow stromal/ |
| | | Mesenchymal stem cells |
| | Collagen II | Chondrocytes |
| | Collagen IV | Chondrocytes |
| | CD29 | Stromal cells |
| | CD44 | Stromal cells |
| | CD73 | Stromal cells |
| | CD166 | Stromal cells |
| | Brachyury | Mesoderm (Notochord) |
| Endoderm | Sox17 | Visceral and definitive |
| Endoderm | Goosecoid (+) | Definitive endoderm |
| | Goosecoid (−) | Visceral endoderm |
| | Albumin | Hepatocytes |
| | B-1 Integrin | Hepatocytes |

TABLE X
CD antigens expression

| CD designation | Gene name | Accession | CM10-1 | B-1 | 4 | CM50-4 | B-16 | 2-2 | 2-1 | B-28 | B-7 | 6-1 | B-25 | B-26 |
|---|---|---|---|---|---|---|---|---|---|---|---|---|---|---|
| CD41 | ITGA2B | NM_000419.2 | 95 | 103 | 117 | 115 | 95 | 98 | 103 | 105 | 120 | 114 | 116 | 99 |
| CD73 | NT5E | NM_002526.1 | 1830 | 1933 | 3846 | 789 | 877 | 1041 | 1531 | 2049 | 1617 | 1852 | 2838 | 3134 |
| CD97 | CD97 (v2) | NM_001784.2 | 1041 | 1378 | 972 | 733 | 950 | 1122 | 1215 | 1906 | 931 | 1135 | 846 | 1035 |
| CD100 | SEMA4D | NM_006378.2 | 180 | 132 | 122 | 129 | 147 | 124 | 121 | 129 | 136 | 215 | 166 | 162 |
| CD107b | INDO | NM_002164.3 | 111 | 108 | 111 | 113 | 111 | 89 | 105 | 113 | 97 | 107 | 110 | 83 |
| CD133 | PROM1 | NM_006017.1 | 108 | 99 | 963 | 74 | 79 | 91 | 87 | 85 | 96 | 93 | 64 | 64 |
| CD140b | PDGFRB | NM_002609.2 | 1653 | 713 | 603 | 3487 | 2428 | 2353 | 3548 | 5164 | 3873 | 6236 | 2020 | 3613 |
| CD151 | CD151 | NM_004357.3 | 1055 | 1030 | 1129 | 525 | 830 | 523 | 1106 | 896 | 516 | 752 | 734 | 1139 |
| CD172A | PTPN61 | NM_080792.1 | 4935 | 1661 | 2295 | 1533 | 1080 | 2912 | 3240 | 1438 | 1303 | 2582 | 1705 | 2077 |
| CD184 | CXCR4 | NM_003467.1 | 107 | 115 | 115 | 102 | 101 | 91 | 107 | 103 | 99 | 97 | 99 | 99 |
| CD225 | IFITM1 | NM_003641.2 | 183 | 222 | 121 | 334 | 1494 | 289 | 475 | 823 | 3601 | 4467 | 1981 | 1964 |
| CD230 | PRNP | NM_183079.1 | 5466 | 4631 | 7840 | 3093 | 7805 | 7995 | 8377 | 5553 | 5130 | 4702 | 7945 | 6262 |
| CD280 | MRC2 | NM_006039.1 | 757 | 806 | 605 | 1275 | 950 | 2331 | 3701 | 3232 | 1889 | 3231 | 2725 | 3257 |
| CD317 | BST2 | NM_004335.2 | 114 | 134 | 123 | 121 | 349 | 107 | 123 | 176 | 225 | 197 | 287 | 191 |
| CD321 | F11R | NM_144501.1 | 163 | 223 | 143 | 98 | 125 | 103 | 101 | 116 | 97 | 112 | 112 | 105 |
| CD324 | CDH1 | NM_004360.2 | 106 | 102 | 163 | 113 | 101 | 108 | 122 | 135 | 91 | 91 | 102 | 104 |
| CD326 | TACSTD1 | NM_002354.1 | 175 | 246 | 190 | 115 | 93 | 104 | 98 | 124 | 99 | 119 | 96 | 112 |
| CD333 | FGFR3 | NM_022965.1 | 150 | 114 | 132 | 118 | 112 | 113 | 117 | 124 | 114 | 102 | 123 | 107 |
| CD334 | FGFR4 | NM_022963.1 | 239 | 160 | 147 | 95 | 90 | 103 | 94 | 107 | 97 | 95 | 100 | 105 |
| CDW210B | IL10RB | NM_000626.3 | 1014 | 674 | 944 | 769 | 1016 | 1322 | 1065 | 1109 | 928 | 1460 | 1046 | 1423 |
| CD designation | Gene name | Accession | B-3 | B-11 | B-2 | B-29 | B-6 | B-17 | B-30 | CM30-2 | CM0-2 | 2-3 | CM10-4 | CM20-4 |
| CD41 | ITGA2B | NM_000419.2 | 100 | 102 | 104 | 139 | 105 | 139 | 121 | 112 | 115 | 93 | 82 | 109 |
| CD73 | NT5E | NM_002526.1 | 1970 | 2235 | 1606 | 291 | 745 | 562 | 2083 | 1681 | 461 | 1320 | 1798 | 1927 |
| CD97 | CD97 (v2) | NM_001784.2 | 979 | 751 | 1415 | 486 | 437 | 1062 | 584 | 573 | 542 | 1051 | 957 | 1281 |
| CD100 | SEMA4D | NM_006378.2 | 152 | 183 | 127 | 316 | 147 | 245 | 154 | 217 | 216 | 115 | 103 | 112 |
| CD107b | INDO | NM_002164.3 | 94 | 105 | 106 | 99 | 103 | 103 | 119 | 112 | 92 | 113 | 111 | 109 |
| CD133 | PROM1 | NM_006017.1 | 87 | 102 | 76 | 67 | 91 | 87 | 88 | 75 | 92 | 75 | 92 | 79 |
| CD140b | PDGFRB | NM_002609.2 | 3708 | 3296 | 5220 | 4920 | 6210 | 6307 | 4437 | 2576 | 3649 | 1741 | 1502 | 1365 |
| CD151 | CD151 | NM_004357.3 | 939 | 1076 | 615 | 832 | 680 | 580 | 761 | 648 | 612 | 912 | 756 | 887 |
| CD172A | PTPNS1 | NM_080792.1 | 1759 | 1542 | 1822 | 1637 | 1201 | 2147 | 1176 | 4232 | 2439 | 3045 | 2900 | 3119 |
| CD184 | CXCR4 | NM_003467.1 | 102 | 106 | 103 | 107 | 97 | 107 | 98 | 98 | 115 | 94 | 101 | 109 |
| CD225 | IFITM1 | NM_003641.2 | 1468 | 1217 | 5077 | 217 | 224 | 417 | 177 | 173 | 152 | 203 | 161 | 176 |
| CD230 | PRNP | NM_183079.1 | 8812 | 5882 | 8971 | 3567 | 4425 | 4211 | 2693 | 5149 | 3754 | 6537 | 8009 | 8736 |
| CD280 | MRC2 | NM_006039.1 | 3287 | 2976 | 2800 | 1532 | 2231 | 2313 | 2013 | 820 | 992 | 1092 | 947 | 1119 |
| CD317 | BST2 | NM_004335.2 | 222 | 192 | 443 | 225 | 131 | 189 | 127 | 160 | 129 | 105 | 113 | 117 |
| CD321 | F11R | NM_144501.1 | 96 | 111 | 118 | 181 | 113 | 118 | 108 | 111 | 126 | 117 | 94 | 104 |
| CD324 | CDH1 | NM_004360.2 | 98 | 123 | 92 | 449 | 107 | 103 | 84 | 121 | 166 | 116 | 127 | 111 |
| CD326 | TACSTD1 | NM_002354.1 | 95 | 104 | 85 | 124 | 107 | 123 | 113 | 162 | 118 | 117 | 121 | 115 |
| CD333 | FGFR3 | NM_022965.1 | 91 | 109 | 103 | 142 | 171 | 173 | 441 | 132 | 257 | 126 | 108 | 116 |
| CD334 | FGFR4 | NM_022963.1 | 86 | 94 | 100 | 155 | 96 | 107 | 107 | 122 | 204 | 97 | 91 | 104 |
| CDW210B | IL10RB | NM_000628.3 | 1197 | 1075 | 1398 | 677 | 615 | 923 | 597 | 760 | 650 | 943 | 695 | 1022 |

| CD designation | Gene name | Accession | CM30-5 | CM50-5 | CM0-5 | CM0-3 | B-14 | H9-B1 | H9-B2 |
|---|---|---|---|---|---|---|---|---|---|
| CD41 | ITGA2B | NM_000419.2 | 101 | 101 | 111 | 114 | 101 | 455 | 471 |
| CD73 | NT5E | NM_002526.1 | 1665 | 1063 | 1297 | 1673 | 682 | 99 | 92 |
| CD97 | CD97 (v2) | NM_001784.2 | 1136 | 1347 | 1114 | 1070 | 719 | 196 | 185 |
| CD100 | SEMA4D | NM_006378.2 | 101 | 138 | 129 | 115 | 105 | 912 | 926 |
| CD107b | INDO | NM_002164.3 | 106 | 97 | 99 | 95 | 92 | 805 | 950 |
| CD133 | PROM1 | NM_006017.1 | 90 | 69 | 80 | 77 | 91 | 511 | 544 |
| CD140b | PDGFRB | NM_002609.2 | 2034 | 3202 | 3744 | 3792 | 701 | 114 | 107 |
| CD151 | CD151 | NM_004357.3 | 854 | 707 | 663 | 853 | 579 | 199 | 189 |
| CD172A | PTPNS1 | NM_080792.1 | 1867 | 1373 | 1287 | 1334 | 1080 | 216 | 227 |
| CD184 | CXCR4 | NM_003467.1 | 109 | 100 | 95 | 104 | 115 | 962 | 1132 |
| CD225 | IFITM1 | NM_003641.2 | 302 | 362 | 457 | 180 | 256 | 9924 | 8642 |
| CD230 | PRNP | NM_183079.1 | 8735 | 5623 | 4548 | 3609 | 3490 | 643 | 632 |
| CD280 | MRC2 | NM_006039.1 | 1223 | 1313 | 1187 | 1072 | 695 | 209 | 215 |
| CD317 | BST2 | NM_004335.2 | 119 | 125 | 116 | 166 | 114 | 229 | 265 |
| CD321 | F11R | NM_144501.1 | 106 | 98 | 96 | 99 | 93 | 750 | 715 |
| CD324 | CDH1 | NM_004360.2 | 118 | 125 | 102 | 98 | 94 | 2630 | 2515 |
| CD326 | TACSTD1 | NM_002354.1 | 117 | 104 | 94 | 109 | 95 | 2647 | 3956 |
| CD333 | FGFR3 | NM_022965.1 | 106 | 105 | 122 | 139 | 103 | 541 | 533 |
| CD334 | FGFR4 | NM_022963.1 | 91 | 96 | 103 | 91 | 89 | 588 | 850 |
| CDW210B | IL10RB | NM_000628.3 | 1000 | 905 | 1103 | 973 | 581 | 164 | 178 |

TABLE XI
CD antigens expression

| CD designation | Gene name | Accession | CM10-1 | B-1 | 4 | CM50-4 | B-16 | 2-2 | 2-1 |
|---|---|---|---|---|---|---|---|---|---|
| CD13 | ANPEP | NM_001150.1 | 108 | 114 | 91 | 945 | 927 | 913 | 1594 |
| CD24 | CD24 | NM_013230.1 | 2095 | 1612 | 670 | 119 | 110 | 139 | 135 |
| CD26 | DPP4 | NM_001935.2 | 171 | 144 | 224 | 206 | 1545 | 1523 | 1183 |
| CD31 | PECAM1 | NM_000442.2 | 123 | 124 | 112 | 109 | 196 | 179 | 201 |
| CD42c | GP1BB | NM_000407.3 | 198 | 172 | 242 | 1528 | 197 | 559 | 432 |
| CD49a | ITGA1 | NM_181501.1 | 134 | 107 | 117 | 153 | 79 | 109 | 100 |
| CD49d | ITGA4 | NM_000885.2 | 86 | 90 | 95 | 215 | 153 | 298 | 409 |
| CD55 | DAF | NM_000574.2 | 423 | 358 | 654 | 475 | 609 | 580 | 941 |
| CD61 | ITGB3 | NM_000212.1 | 413 | 380 | 276 | 108 | 116 | 121 | 137 |
| CD70 | TNFSF7 | NM_001252.2 | 237 | 417 | 154 | 117 | 143 | 163 | 215 |
| CD71 | TFRC | NM_003234.1 | 498 | 638 | 504 | 223 | 567 | 229 | 349 |
| CD75 | ST6GAL1 | NM_173217.1 | 353 | 288 | 524 | 210 | 122 | 157 | 159 |
| CD77 | A4GALT | NM_017436.3 | 150 | 131 | 150 | 174 | 289 | 167 | 177 |
| CD83 | CD83 | NM_004233.2 | 157 | 201 | 145 | 45 | 107 | 115 | 135 |
| CD87 | PLAUR | NM_002659.1 | 1180 | 522 | 250 | 252 | 202 | 203 | 191 |
| CD90 | THY1 | NM_006288.2 | 243 | 384 | 153 | 643 | 1196 | 691 | 1387 |
| CD106 | VCAM1 | NM_001078.2 | 336 | 721 | 122 | 190 | 154 | 108 | 114 |
| CD117 | KIT | NM_000222.1 | 182 | 130 | 188 | 120 | 110 | 103 | 100 |
| CD118 | LIFR | NM_002310.2 | 102 | 102 | 86 | 115 | 140 | 112 | 124 |
| CD120B | TNFRSF1B | NM_001066.2 | 106 | 100 | 109 | 119 | 157 | 121 | 129 |
| CD121a | IL1R1 | NM_000877.2 | 159 | 179 | 119 | 450 | 3154 | 502 | 859 |
| CD127 | IL7R | NM_002185.2 | 163 | 121 | 131 | 114 | 133 | 115 | 117 |
| CD133 | PROM1 | NM_006017.1 | 108 | 99 | 983 | 74 | 79 | 91 | 87 |
| CD140a | PDGFRA | NM_006206.2 | 125 | 98 | 179 | 695 | 749 | 346 | 642 |
| CD141 | THBD | NM_000361.2 | 618 | 461 | 694 | 125 | 640 | 95 | 101 |
| CD142 | F3 | NM_001993.2 | 1587 | 2495 | 1638 | 102 | 275 | 121 | 132 |
| CD155 | PVR | NM_006505.2 | 465 | 357 | 474 | 63 | 142 | 307 | 490 |
| CDw156c | ADAM10 | NM_001110.1 | 711 | 427 | 421 | 358 | 459 | 370 | 373 |
| CD157 | BST1 | NM_004334.1 | 167 | 160 | 146 | 153 | 441 | 580 | 447 |
| CD164 | CD164 | NM_006016.3 | 1253 | 570 | 459 | 832 | 463 | 152 | 143 |
| CD166 | ALCAM | NM_001627.1 | 793 | 461 | 410 | 145 | 329 | 118 | 160 |
| CD202b | TEK | NM_000459.1 | 134 | 105 | 105 | 38 | 315 | 2146 | 2764 |
| CD208 | LAMP3 | NM_014398.2 | 91 | 97 | 99 | 290 | 115 | 273 | 396 |
| CD213A2 | IL13RA2 | NM_000640.2 | 105 | 104 | 122 | 99 | 238 | 112 | 99 |
| CDw217 | IL17R | NM_014339.3 | 127 | 117 | 115 | 117 | 105 | 112 | 135 |
| CDW218A | IL18R1 | NM_003855.2 | 102 | 194 | 109 | 132 | 166 | 124 | 107 |
| CD221 | IGF1R | NM_000875.2 | 144 | 146 | 148 | 158 | 138 | 156 | 241 |
| CD225 | IFITM1 | NM_003641.2 | 183 | 222 | 121 | 334 | 1494 | 289 | 475 |
| CD227 | MUC1 | NM_002456.3 | 128 | 122 | 135 | 172 | 159 | 167 | 225 |
| CD227 | MUC1 | NM_182741.1 | 117 | 114 | 106 | 137 | 109 | 121 | 165 |
| CD243 | ABCB1 | NM_000927.3 | 354 | 280 | 407 | 114 | 101 | 115 | 103 |
| CD249 | ENPEP | NM_001977.2 | 126 | 128 | 105 | 106 | 114 | 107 | 118 |
| CD252 | TNFSF4 | NM_003326.2 | 209 | 174 | 164 | 180 | 126 | 444 | 350 |
| CD253 | TNFSF10 | NM_003810.2 | 387 | 712 | 101 | 107 | 124 | 94 | 101 |
| CD264 | TNFRSF10D | NM_003840.3 | 327 | 465 | 426 | 162 | 129 | 169 | 208 |
| CD273 | PDCD1LG2 | NM_025239.2 | 207 | 243 | 230 | 126 | 118 | 135 | 153 |
| CD282 | TLR2 | NM_003264.2 | 224 | 426 | 148 | 110 | 114 | 100 | 99 |
| CD284 | TLR4 | NM_138557.1 | 196 | 245 | 219 | 126 | 114 | 138 | 108 |
| CD317 | BST2 | NM_004335.2 | 114 | 134 | 123 | 121 | 349 | 107 | 123 |
| CD318 | CDCP1 | NM_022842.3 | 274 | 589 | 308 | 118 | 133 | 113 | 112 |
| CD326 | TACSTD1 | NM_002354.1 | 175 | 246 | 190 | 115 | 93 | 104 | 98 |
| CD333 | FGFR3 | NM_022965.1 | 150 | 114 | 132 | 118 | 112 | 113 | 117 |
| CD334 | FGFR4 | NM_022963.1 | 239 | 160 | 147 | 95 | 90 | 103 | 94 |
| CD339 | JAG1 | NM_000214.1 | 608 | 468 | 519 | 194 | 163 | 160 | 165 |
| CD designation | Gene name | Accession | B-28 | B-7 | 6-1 | B-25 | B-26 | B-3 | B-11 |
| CD13 | ANPEP | NM_001150.1 | 1023 | 925 | 1431 | 1635 | 2306 | 2043 | 1902 |
| CD24 | CD24 | NM_013230.1 | 334 | 105 | 102 | 111 | 103 | 92 | 101 |
| CD26 | DPP4 | NM_001935.2 | 160 | 1181 | 828 | 1903 | 1194 | 1501 | 597 |
| CD31 | PECAM1 | NM_000442.2 | 122 | 153 | 138 | 132 | 138 | 223 | 158 |
| CD42c | GP1BB | NM_000407.3 | 2603 | 578 | 752 | 241 | 294 | 352 | 521 |
| CD49a | ITGA1 | NM_181501.1 | 235 | 74 | 92 | 89 | 95 | 84 | 89 |
| CD49d | ITGA4 | NM_000885.2 | 309 | 116 | 125 | 146 | 134 | 163 | 195 |
| CD55 | DAF | NM_000574.2 | 470 | 304 | 385 | 598 | 663 | 623 | 566 |
| CD61 | ITGB3 | NM_000212.1 | 127 | 113 | 126 | 138 | 132 | 129 | 122 |
| CD70 | TNFSF7 | NM_001252.2 | 225 | 190 | 397 | 761 | 869 | 463 | 510 |
| CD71 | TFRC | NM_003234.1 | 635 | 268 | 208 | 818 | 567 | 676 | 468 |
| CD75 | ST6GAL1 | NM_173217.1 | 182 | 120 | 186 | 152 | 143 | 156 | 159 |
| CD77 | A4GALT | NM_017436.3 | 191 | 372 | 372 | 323 | 421 | 344 | 243 |
| CD83 | CD83 | NM_004233.2 | 106 | 130 | 117 | 116 | 124 | 115 | 108 |
| CD87 | PLAUR | NM_002659.1 | 176 | 112 | 176 | 175 | 203 | 169 | 179 |
| CD90 | THY1 | NM_006288.2 | 1516 | 497 | 1678 | 908 | 1356 | 1138 | 1224 |
| CD106 | VCAM1 | NM_001078.2 | 157 | 151 | 147 | 144 | 127 | 126 | 131 |
| CD117 | KIT | NM_000222.1 | 180 | 131 | 137 | 161 | 126 | 120 | 141 |
| CD118 | LIFR | NM_002310.2 | 151 | 147 | 245 | 211 | 218 | 180 | 190 |
| CD120B | TNFRSF1B | NM_001066.2 | 107 | 178 | 210 | 218 | 214 | 135 | 128 |
| CD121a | IL1R1 | NM_000877.2 | 657 | 2043 | 5257 | 3141 | 4413 | 3680 | 1965 |
| CD127 | IL7R | NM_002185.2 | 119 | 129 | 122 | 128 | 136 | 120 | 133 |
| CD133 | PROM1 | NM_006017.1 | 85 | 96 | 93 | 84 | 84 | 87 | 102 |
| CD140a | PDGFRA | NM_006206.2 | 976 | 2873 | 3383 | 1565 | 2493 | 1910 | 1510 |
| CD141 | THBD | NM_000361.2 | 144 | 174 | 285 | 446 | 368 | 136 | 164 |
| CD142 | F3 | NM_001993.2 | 111 | 98 | 165 | 159 | 169 | 128 | 154 |
| CD155 | PVR | NM_006505.2 | 332 | 153 | 246 | 288 | 308 | 310 | 330 |
| CDw156c | ADAM10 | NM_001110.1 | 325 | 243 | 290 | 307 | 363 | 351 | 344 |
| CD157 | BST1 | NM_004334.1 | 222 | 234 | 633 | 466 | 551 | 289 | 480 |
| CD164 | CD164 | NM_006016.3 | 153 | 130 | 190 | 127 | 140 | 149 | 148 |
| CD166 | ALCAM | NM_001627.1 | 166 | 133 | 179 | 202 | 186 | 173 | 184 |
| CD202b | TEK | NM_000459.1 | 1991 | 553 | 426 | 644 | 1031 | 1158 | 1639 |
| CD208 | LAMP3 | NM_014398.2 | 218 | 99 | 99 | 116 | 121 | 124 | 242 |
| CD213A2 | IL13RA2 | NM_000640.2 | 99 | 175 | 179 | 355 | 298 | 170 | 152 |
| CDw217 | IL17R | NM_014339.3 | 133 | 135 | 138 | 143 | 142 | 138 | 120 |
| CDW218A | IL18R1 | NM_003855.2 | 115 | 303 | 382 | 296 | 328 | 393 | 244 |
| CD221 | IGF1R | NM_000875.2 | 233 | 153 | 206 | 193 | 232 | 225 | 250 |
| CD225 | IFITM1 | NM_003641.2 | 823 | 3601 | 4467 | 1981 | 1964 | 1468 | 1217 |
| CD227 | MUC1 | NM_002456.3 | 289 | 229 | 396 | 326 | 343 | 311 | 261 |
| CD227 | MUC1 | NM_182741.1 | 185 | 145 | 226 | 193 | 208 | 213 | 195 |
| CD243 | ABCB1 | NM_000927.3 | 130 | 101 | 104 | 106 | 106 | 104 | 107 |
| CD249 | ENPEP | NM_001977.2 | 183 | 108 | 100 | 104 | 115 | 114 | 107 |
| CD252 | TNFSF4 | NM_003326.2 | 247 | 145 | 183 | 171 | 180 | 213 | 192 |
| CD253 | TNFSF10 | NM_003810.2 | 111 | 121 | 203 | 134 | 163 | 119 | 128 |
| CD264 | TNFRSF10D | NM_003840.3 | 181 | 126 | 146 | 164 | 176 | 160 | 188 |
| CD273 | PDCD1LG2 | NM_025239.2 | 204 | 131 | 129 | 137 | 123 | 145 | 130 |
| CD282 | TLR2 | NM_003264.2 | 116 | 117 | 130 | 127 | 119 | 114 | 109 |
| CD284 | TLR4 | NM_138557.1 | 152 | 136 | 177 | 123 | 146 | 130 | 132 |
| CD317 | BST2 | NM_004335.2 | 176 | 225 | 197 | 287 | 191 | 222 | 192 |
| CD318 | CDCP1 | NM_022842.3 | 223 | 160 | 172 | 261 | 268 | 193 | 112 |
| CD326 | TACSTD1 | NM_002354.1 | 124 | 99 | 119 | 96 | 112 | 95 | 104 |
| CD333 | FGFR3 | NM_022965.1 | 124 | 114 | 102 | 123 | 107 | 91 | 109 |
| CD334 | FGFR4 | NM_022963.1 | 107 | 97 | 95 | 100 | 105 | 86 | 94 |
| CD339 | JAG1 | NM_000214.1 | 429 | 221 | 283 | 207 | 255 | 278 | 265 |
| CD designation | Gene name | Accession | B-2 | B-29 | B-6 | B-17 | B-30 | CM30-2 | CM0-2 |
| CD13 | ANPEP | NM_001150.1 | 1970 | 122 | 197 | 183 | 190 | 155 | 116 |
| CD24 | CD24 | NM_013230.1 | 107 | 3564 | 152 | 267 | 126 | 198 | 3247 |
| CD26 | DPP4 | NM_001935.2 | 968 | 152 | 110 | 157 | 103 | 272 | 257 |
| CD31 | PECAM1 | NM_000442.2 | 166 | 112 | 108 | 136 | 125 | 128 | 108 |
| CD42c | GP1BB | NM_000407.3 | 531 | 1336 | 5504 | 3628 | 8758 | 748 | 980 |
| CD49a | ITGA1 | NM_181501.1 | 91 | 101 | 104 | 137 | 288 | 92 | 96 |
| CD49d | ITGA4 | NM_000885.2 | 108 | 81 | 99 | 107 | 138 | 91 | 93 |
| CD55 | DAF | NM_000574.2 | 697 | 287 | 556 | 467 | 421 | 554 | 393 |
| CD61 | ITGB3 | NM_000212.1 | 127 | 160 | 134 | 117 | 133 | 121 | 236 |
| CD70 | TNFSF7 | NM_001252.2 | 316 | 1178 | 303 | 781 | 106 | 701 | 376 |
| CD71 | TFRC | NM_003234.1 | 328 | 550 | 443 | 451 | 579 | 242 | 420 |
| CD75 | ST6GAL1 | NM_173217.1 | 118 | 400 | 366 | 411 | 371 | 381 | 298 |
| CD77 | A4GALT | NM_017436.3 | 618 | 165 | 110 | 136 | 108 | 123 | 142 |
| CD83 | CD83 | NM_004233.2 | 116 | 195 | 151 | 123 | 130 | 123 | 146 |
| CD87 | PLAUR | NM_002659.1 | 203 | 112 | 143 | 126 | 104 | 415 | 381 |
| CD90 | THY1 | NM_006288.2 | 1198 | 683 | 749 | 596 | 156 | 717 | 459 |
| CD106 | VCAM1 | NM_001078.2 | 115 | 216 | 123 | 124 | 140 | 111 | 627 |
| CD117 | KIT | NM_000222.1 | 123 | 272 | 515 | 169 | 337 | 250 | 215 |
| CD118 | LIFR | NM_002310.2 | 208 | 113 | 136 | 113 | 84 | 105 | 116 |
| CD120B | TNFRSF1B | NM_001066.2 | 237 | 104 | 110 | 100 | 121 | 98 | 102 |
| CD121a | IL1R1 | NM_000877.2 | 4147 | 200 | 174 | 219 | 154 | 123 | 186 |
| CD127 | IL7R | NM_002185.2 | 137 | 142 | 106 | 120 | 111 | 171 | 127 |
| CD133 | PROM1 | NM_006017.1 | 76 | 87 | 91 | 87 | 88 | 75 | 92 |
| CD140a | PDGFRA | NM_006206.2 | 2969 | 373 | 1278 | 1744 | 1370 | 991 | 278 |
| CD141 | THBD | NM_000361.2 | 350 | 675 | 1483 | 1438 | 4751 | 1309 | 847 |
| CD142 | F3 | NM_001993.2 | 98 | 120 | 102 | 112 | 91 | 690 | 208 |
| CD155 | PVR | NM_006505.2 | 176 | 294 | 256 | 270 | 261 | 203 | 182 |
| CDw156c | ADAM10 | NM_001110.1 | 351 | 302 | 446 | 383 | 228 | 754 | 744 |
| CD157 | BST1 | NM_004334.1 | 479 | 152 | 201 | 195 | 242 | 199 | 150 |
| CD164 | CD164 | NM_006016.3 | 159 | 176 | 154 | 174 | 131 | 1364 | 967 |
| CD166 | ALCAM | NM_001627.1 | 139 | 313 | 435 | 276 | 311 | 754 | 679 |
| CD202b | TEK | NM_000459.1 | 917 | 94 | 346 | 710 | 1082 | 156 | 146 |
| CD208 | LAMP3 | NM_014398.2 | 94 | 200 | 405 | 374 | 232 | 113 | 148 |
| CD213A2 | ILI3RA2 | NM_000640.2 | 308 | 90 | 100 | 106 | 93 | 93 | 87 |
| CDw217 | IL17R | NM_014339.3 | 146 | 136 | 164 | 167 | 137 | 127 | 128 |
| CDW218A | IL18R1 | NM_003855.2 | 443 | 114 | 101 | 98 | 80 | 95 | 115 |
| CD221 | IGF1R | NM_000875.2 | 187 | 271 | 334 | 323 | 419 | 184 | 149 |
| CD225 | IFITM1 | NM_003641.2 | 5077 | 217 | 224 | 417 | 177 | 173 | 152 |
| CD227 | MUC1 | NM_002456.3 | 308 | 232 | 214 | 300 | 182 | 185 | 149 |
| CD227 | MUC1 | NM_182741.1 | 192 | 147 | 155 | 181 | 146 | 129 | 102 |
| CD243 | ABCB1 | NM_000927.3 | 95 | 239 | 102 | 105 | 103 | 112 | 224 |
| CD249 | ENPEP | NM_001977.2 | 100 | 104 | 100 | 117 | 92 | 102 | 132 |
| CD252 | TNFSF4 | NM_003326.2 | 132 | 214 | 216 | 230 | 213 | 235 | 169 |
| CD253 | TNFSF10 | NM_003810.2 | 144 | 142 | 104 | 100 | 106 | 98 | 145 |
| CD264 | TNFRSF10D | NM_003840.3 | 135 | 165 | 256 | 156 | 160 | 370 | 321 |
| CD273 | PDCD1LG2 | NM_025239.2 | 124 | 106 | 128 | 113 | 148 | 230 | 137 |
| CD282 | TLR2 | NM_003264.2 | 132 | 171 | 141 | 190 | 112 | 113 | 122 |
| CD284 | TLR4 | NM_138557.1 | 159 | 111 | 149 | 146 | 193 | 175 | 124 |
| CD317 | BST2 | NM_004335.2 | 443 | 225 | 131 | 189 | 127 | 160 | 129 |
| CD318 | CDCP1 | NM_022842.3 | 112 | 166 | 101 | 137 | 122 | 115 | 165 |
| CD326 | TACSTD1 | NM_002354.1 | 85 | 124 | 107 | 123 | 113 | 162 | 118 |
| CD333 | FGFR3 | NM_022965.1 | 103 | 142 | 171 | 173 | 441 | 132 | 257 |
| CD334 | FGFR4 | NM_022963.1 | 100 | 155 | 96 | 107 | 107 | 122 | 204 |
| CD339 | JAG1 | NM_000214.1 | 172 | 725 | 615 | 330 | 1715 | 247 | 396 |

| CD designation | Gene name | Accession | 2-3 | CM10-4 | CM20-4 | CM30-5 | CM50-5 | CM0-5 |
|---|---|---|---|---|---|---|---|---|
| CD13 | ANPEP | NM_001150.1 | 507 | 746 | 1084 | 1329 | 636 | 1483 |
| CD24 | CD24 | NM_013230.1 | 112 | 101 | 348 | 110 | 241 | 106 |
| CD26 | DPP4 | NM_001935.2 | 279 | 1191 | 847 | 845 | 227 | 307 |
| CD31 | PECAM1 | NM_000442.2 | 130 | 136 | 152 | 161 | 142 | 131 |
| CD42c | GP1BB | NM_000407.3 | 976 | 641 | 225 | 578 | 2273 | 1687 |
| CD49a | ITGA1 | NM_181501.1 | 92 | 105 | 89 | 90 | 100 | 126 |
| CD49d | ITGA4 | NM_000885.2 | 235 | 521 | 347 | 333 | 454 | 272 |
| CD55 | DAF | NM_000574.2 | 663 | 1908 | 1665 | 738 | 610 | 577 |
| CD61 | ITGB3 | NM_000212.1 | 119 | 129 | 136 | 123 | 120 | 125 |
| CD70 | TNFSF7 | NM_001252.2 | 102 | 105 | 274 | 140 | 124 | 131 |
| CD71 | TFRC | NM_003234.1 | 176 | 250 | 398 | 313 | 326 | 320 |
| CD75 | ST6GAL1 | NM_173217.1 | 174 | 191 | 144 | 161 | 213 | 172 |
| CD77 | A4GALT | NM_017436.3 | 144 | 150 | 225 | 145 | 150 | 204 |
| CD83 | CD83 | NM_004233.2 | 109 | 108 | 118 | 121 | 118 | 110 |
| CD87 | PLAUR | NM_002659.1 | 348 | 486 | 1066 | 812 | 397 | 375 |
| CD90 | THY1 | NM_006288.2 | 1009 | 1027 | 1502 | 1894 | 1187 | 1014 |
| CD106 | VCAM1 | NM_001078.2 | 120 | 130 | 109 | 159 | 212 | 173 |
| CD117 | KIT | NM_000222.1 | 162 | 122 | 98 | 126 | 103 | 181 |
| CD118 | LIFR | NM_002310.2 | 104 | 106 | 109 | 110 | 123 | 130 |
| CD120B | TNFRSF1B | NM_001066.2 | 105 | 103 | 109 | 112 | 98 | 112 |
| CD121a | IL1R1 | NM_000877.2 | 182 | 219 | 386 | 631 | 384 | 787 |
| CD127 | IL7R | NM_002185.2 | 117 | 112 | 110 | 119 | 97 | 110 |
| CD133 | PROM1 | NM_006017.1 | 75 | 92 | 79 | 90 | 69 | 80 |
| CD140a | PDGFRA | NM_006206.2 | 360 | 406 | 642 | 800 | 638 | 668 |
| CD141 | THBD | NM_000361.2 | 108 | 97 | 228 | 118 | 98 | 116 |
| CD142 | F3 | NM_001993.2 | 229 | 116 | 778 | 121 | 212 | 120 |
| CD155 | PVR | NM_006505.2 | 236 | 309 | 204 | 266 | 225 | 210 |
| CDw156c | ADAM10 | NM_001110.1 | 336 | 424 | 489 | 493 | 508 | 442 |
| CD157 | BST1 | NM_004334.1 | 482 | 718 | 450 | 495 | 229 | 185 |
| CD164 | CD164 | NM_006016.3 | 413 | 784 | 1163 | 1185 | 973 | 1115 |
| CD166 | ALCAM | NM_001627.1 | 264 | 370 | 278 | 349 | 258 | 257 |
| CD202b | TEK | NM_000459.1 | 1119 | 1857 | 2505 | 1740 | 1982 | 953 |
| CD208 | LAMP3 | NM_014398.2 | 186 | 180 | 153 | 166 | 400 | 212 |
| CD213A2 | IL13RA2 | NM_000640.2 | 104 | 103 | 98 | 100 | 85 | 101 |
| CDw217 | IL17R | NM_014339.3 | 95 | 115 | 122 | 120 | 116 | 115 |
| CDW218A | IL18R1 | NM_003855.2 | 99 | 155 | 105 | 135 | 110 | 131 |
| CD221 | IGF1R | NM_000875.2 | 140 | 136 | 125 | 134 | 158 | 160 |
| CD225 | IFITM1 | NM_003641.2 | 203 | 181 | 178 | 302 | 362 | 457 |
| CD227 | MUC1 | NM_002456.3 | 122 | 116 | 164 | 150 | 215 | 191 |
| CD227 | MUC1 | NM_182741.1 | 117 | 119 | 119 | 116 | 142 | 135 |
| CD243 | ABCB1 | NM_000927.3 | 114 | 109 | 113 | 102 | 106 | 92 |
| CD249 | ENPEP | NM_001977.2 | 114 | 107 | 108 | 91 | 108 | 103 |
| CD252 | TNFSF4 | NM_003326.2 | 170 | 152 | 179 | 180 | 228 | 203 |
| CD253 | TNFSF10 | NM_003810.2 | 91 | 88 | 113 | 100 | 119 | 103 |
| CD264 | TNFRSF10D | NM_003840.3 | 324 | 319 | 456 | 286 | 276 | 203 |
| CD273 | PDCD1LG2 | NM_025239.2 | 189 | 229 | 218 | 210 | 193 | 152 |
| CD282 | TLR2 | NM_003264.2 | 116 | 108 | 106 | 97 | 111 | 101 |
| CD284 | TLR4 | NM_138557.1 | 143 | 193 | 207 | 189 | 182 | 146 |
| CD317 | BST2 | NM_004335.2 | 105 | 113 | 117 | 119 | 125 | 116 |
| CD318 | CDCP1 | NM_022842.3 | 140 | 103 | 139 | 134 | 130 | 108 |
| CD326 | TACSTD1 | NM_002354.1 | 117 | 121 | 115 | 117 | 104 | 94 |
| CD333 | FGFR3 | NM_022965.1 | 126 | 106 | 116 | 106 | 105 | 122 |
| CD334 | FGFR4 | NM_022963.1 | 97 | 91 | 104 | 91 | 96 | 103 |
| CD339 | JAG1 | NM_000214.1 | 200 | 161 | 157 | 195 | 167 | 257 |

| CD designation | Gene name | Accession | CM0-3 | B-14 | H9-B1 | H9-B2 |
|---|---|---|---|---|---|---|
| CD13 | ANPEP | NM_001150.1 | 816 | 404 | 94 | 93 |
| CD24 | CD24 | NM_013230.1 | 102 | 115 | 7698 | 9263 |
| CD26 | DPP4 | NM_001935.2 | 134 | 592 | 160 | 136 |
| CD31 | PECAM1 | NM_000442.2 | 126 | 266 | 109 | 105 |
| CD42c | GP1BB | NM_000407.3 | 3673 | 207 | 250 | 237 |
| CD49a | ITGA1 | NM_181501.1 | 235 | 96 | 87 | 98 |
| CD49d | ITGA4 | NM_000885.2 | 262 | 201 | 87 | 93 |
| CD55 | DAF | NM_000574.2 | 537 | 331 | 285 | 318 |
| CD61 | ITGB3 | NM_000212.1 | 128 | 116 | 100 | 92 |
| CD70 | TNFSF7 | NM_001252.2 | 168 | 104 | 106 | 111 |
| CD71 | TFRC | NM_003234.1 | 264 | 197 | 1626 | 1760 |
| CD75 | ST6GAL1 | NM_173217.1 | 157 | 113 | 801 | 839 |
| CD77 | A4GALT | NM_017436.3 | 242 | 131 | 157 | 166 |
| CD83 | CD83 | NM_004233.2 | 114 | 91 | 152 | 153 |
| CD87 | PLAUR | NM_002659.1 | 413 | 250 | 98 | 127 |
| CD90 | THY1 | NM_006288.2 | 865 | 652 | 253 | 322 |
| CD106 | VCAM1 | NM_001078.2 | 219 | 94 | 88 | 119 |
| CD117 | KIT | NM_000222.1 | 166 | 104 | 289 | 348 |
| CD118 | LIFR | NM_002310.2 | 112 | 129 | 94 | 95 |
| CD120B | TNFRSF1B | NM_001066.2 | 103 | 107 | 97 | 107 |
| CD121a | IL1R1 | NM_000877.2 | 298 | 282 | 89 | 110 |
| CD127 | IL7R | NM_002185.2 | 104 | 124 | 102 | 105 |
| CD133 | PROM1 | NM_006017.1 | 77 | 91 | 511 | 544 |
| CD140a | PDGFRA | NM_006206.2 | 281 | 285 | 97 | 112 |
| CD141 | THBD | NM_000361.2 | 180 | 123 | 97 | 107 |
| CD142 | F3 | NM_001993.2 | 497 | 143 | 143 | 191 |
| CD155 | PVR | NM_006505.2 | 175 | 142 | 114 | 124 |
| CDw156c | ADAM10 | NM_001110.1 | 395 | 260 | 226 | 315 |
| CD157 | BST1 | NM_004334.1 | 175 | 375 | 90 | 101 |
| CD164 | CD164 | NM_006016.3 | 1162 | 243 | 238 | 446 |
| CD166 | ALCAM | NM_001627.1 | 241 | 209 | 126 | 141 |
| CD202b | TEK | NM_000459.1 | 961 | 729 | 175 | 209 |
| CD208 | LAMP3 | NM_014398.2 | 154 | 96 | 132 | 131 |
| CD213A2 | IL13RA2 | NM_000640.2 | 96 | 99 | 95 | 93 |
| CDw217 | IL17R | NM_014339.3 | 121 | 111 | 115 | 113 |
| CDW218A | IL18R1 | NM_003855.2 | 149 | 108 | 87 | 111 |
| CD221 | IGF1R | NM_000875.2 | 161 | 127 | 177 | 174 |
| CD225 | IFITM1 | NM_003641.2 | 180 | 256 | 9924 | 8642 |
| CD227 | MUC1 | NM_002456.3 | 154 | 93 | 115 | 102 |
| CD227 | MUC1 | NM_182741.1 | 130 | 109 | 100 | 101 |
| CD243 | ABCB1 | NM_000927.3 | 90 | 111 | 95 | 95 |
| CD249 | ENPEP | NM_001977.2 | 162 | 115 | 105 | 112 |
| CD252 | TNFSF4 | NM_003326.2 | 197 | 218 | 123 | 126 |
| CD253 | TNFSF10 | NM_003810.2 | 108 | 108 | 101 | 105 |
| CD264 | TNFRSF10D | NM_003840.3 | 237 | 229 | 107 | 113 |
| CD273 | PDCD1LG2 | NM_025239.2 | 192 | 133 | 94 | 73 |
| CD282 | TLR2 | NM_003264.2 | 94 | 86 | 109 | 120 |
| CD284 | TLR4 | NM_138557.1 | 168 | 135 | 115 | 98 |
| CD317 | BST2 | NM_004335.2 | 166 | 114 | 229 | 265 |
| CD318 | CDCP1 | NM_022842.3 | 109 | 132 | 132 | 118 |
| CD326 | TACSTD1 | NM_002354.1 | 109 | 95 | 2647 | 3956 |
| CD333 | FGFR3 | NM_022965.1 | 139 | 103 | 541 | 533 |
| CD334 | FGFR4 | NM_022963.1 | 91 | 89 | 588 | 850 |
| CD339 | JAG1 | NM_000214.1 | 513 | 114 | 165 | 168 |

TABLE XII
Single Cell-Derived Cell Lines of Series 1 and 2

| Series 1 Exp. | | | Series 2 Exp. | | |
|---|---|---|---|---|---|
| Line | ACTC | | Line | ACTC | |
| Name | No. | Medium | Name | No. | Medium |
| 1 | | DMEM 10% Fetal | CM0-1 | | DMEM 10% |
| 2 | | Bovine Serum | CM0-2 | 77 | Fetal Bovine |
| 3 | | | CM0-3 | 73 | Serum |
| 4 | | | CM0-4 | | |
| 5 | | | CM0-5 | 74 | |
| 6 | | | CM10-1 | | |
| B-1 | | | CM10-2 | | |
| B-2 | 51 | | CM10-3 | | |
| B-3 | 55 | | CM10-4 | | |
| B-4 | 66 | | CM20-1 | | |
| B-5 | | | CM20-2 | | |
| B-6 | 56 | | CM20-3 | | |
| B-7 | 53 | | CM20-4 | 79 | |
| B-9 | | | CM20-5 | | |
| B-10 | | | CM30-1 | | |
| B-11 | 58 | | CM30-2 | 78 | |
| B-12 | 65 | | CM30-3 | | |
| B-13 | | | CM30-4 | | |
| B-14 | 67 | | CM30-5 | | |
| B-15 | 71 | | CM50-1 | | |
| B-16 | 59 | | CM50-2 | 76 | |
| B-17 | 54 | | CM50-3 | | |
| B-18 | | | CM50-4 | 72 | |
| B-19 | | | CM50-5 | 75 | |

| | |
|---|---|
| B-20 | TOTAL COLONIES |
| B-21 | SERIES 2 = 24 |

| | |
|---|---|
| B-22 | |
| B-23 | |
| B-24 | |
| B-25 | 57 |
| B-26 | 50 |
| B-27 | |
| B-28 | 60 |
| B-29 | 52 |
| B-30 | 61 |
| B-31 | |
| B-32 | |
| B-33 | |
| B-34 | |
| B-35 | |
| 2-1 | 63 |
| 2-2 | 62 |
| 2-3 | 70 |
| 2-4 | |
| 4-1 | |
| 4-2 | 69 |
| 4-3 | |
| 4-4 | |
| 5-1 | |
| 5-2 | |
| 5-3 | |
| 5-4 | 68 |
| 5-5 | |
| 6-1 | 64 |

TOTAL COLONIES
SERIES 1 = 54

What is claimed is:

1. A progenitor cell line capable of propagating in vitro for at least 20 doublings, wherein said progenitor cell line has a gene expression profile similar to any cell line in Tables XX to XXIV.

2. The progenitor cell line of claim 1, wherein said cell line is clonal.

3. The progenitor cell line of claim 1, wherein said cell line is oligoclonal.

4. The progenitor cell line of claim 1, wherein said cell line is polyclonal.

5. The progenitor cell line of claim 1, wherein said progenitor cell line is a human progenitor cell line.

6. The progenitor cell line of claim 1, wherein the progenitor cell line is derived from an ES cell or an iPS cell.

7. The progenitor cell line of claim 1, wherein the gene expression profile is maintained for at least 100 doublings.

8. The progenitor cell line of claim 1, wherein the progenitor cell line is selected from the cell lines listed in Table XX.

9. A method for determining the differentiation potential of a progenitor cell line comprising the steps of:
i. culturing the progenitor cell line under one or more culture conditions, wherein said one or more culture conditions is selected from Table 1; and
ii. determining a gene expression pattern in each of said progenitor cell line cultures to obtain gene expression results; and
iii. analyzing the gene expression results for markers of cell differentiation, thereby determining the differentiation potential of the progenitor cell line.

10. The method of claim 9, wherein the culturing step comprises culturing the progenitor cell line in micromass culture conditions.

11. The method of claim 9, wherein the culturing step comprises culturing the progenitor cell line in ovo.

12. The method of claim 9, wherein the culturing step comprises culturing the progenitor cell line in vivo.


SEQUENCE LISTING (22 sequences)

SEQ ID NO: 1 - protein, 11 aa, Artificial Sequence
  Synthetic peptide
YGRKKRRQRRR

SEQ ID NO: 2 - protein, 9 aa, Artificial Sequence
  Synthetic peptide
RKKRRQRRR

SEQ ID NO: 3 - protein, 11 aa, Artificial Sequence
  Synthetic peptide
YARKARRQARR

SEQ ID NO: 4 - protein, 12 aa, Artificial Sequence
  Synthetic peptide
YARZLAARQARA

SEQ ID NO: 5 - protein, 11 aa, Artificial Sequence
  Synthetic peptide
YARAARRAARR

SEQ ID NO: 6 - protein, 11 aa, Artificial Sequence
  Synthetic peptide
RARAARRAARA

SEQ ID NO: 7 - protein, 15 aa, Artificial Sequence
  Synthetic peptide
RQIKIWFQNRRMKWM

SEQ ID NO: 8 - protein, 34 aa, Artificial Sequence
  Synthetic peptide
DAATATRGRSAASRPTERPRAPARSASRPRRPVE

SEQ ID NO: 9 - protein, 7 aa, Artificial Sequence
  Synthetic peptide
RRRRRRR

SEQ ID NO: 10 - protein, 11 aa, Artificial Sequence
  Synthetic peptide
YARAAARQARA

SEQ ID NO: 11 - protein, 11 aa, Artificial Sequence
  Synthetic peptide
YARARARQARA

SEQ ID NO: 12 - RNA, 22 nt, Artificial Sequence
  Synthetic miRNA
uagcagcacguaaauauuggcg

SEQ ID NO: 13 - DNA, 44 bp, Artificial Sequence
  Synthetic primer
ctcaactggtgtcgtggagtcggcaattcagttgagcgccaata

SEQ ID NO: 14 - DNA, 30 bp, Artificial Sequence
  Synthetic primer
acactccagctgggtagcagcacgtaaata

SEQ ID NO: 15 - DNA, 18 bp, Artificial Sequence
  Synthetic probe
ttcagttgagcgccaata

SEQ ID NO: 16 - RNA, 22 nt, Artificial Sequence
  Synthetic miRNA
agugccgcagaguuuguagugu

SEQ ID NO: 17 - DNA, 44 bp, Artificial Sequence
  Synthetic primer
ctcaactggtgtcgtggagtcggcaattcagttgagacactaca

SEQ ID NO: 18 - DNA, 30 bp, Artificial Sequence
  Synthetic primer
acactccagctgggagtgccgcagagtttg

SEQ ID NO: 19 - DNA, 18 bp, Artificial Sequence
  Synthetic probe
ttcagttgagacactaca

SEQ ID NO: 20 - DNA, 48 bp, Artificial Sequence
  Synthetic primer
atatggatccggcgcgccgtcgactttttttttttttttttttttttt

SEQ ID NO: 21 - DNA, 48 bp, Artificial Sequence
  Synthetic primer
atatctcgagggcgcgccggatcctttttttttttttttttttttttt

SEQ ID NO: 22 - DNA, 63 bp, Artificial Sequence
  Synthetic primer
ggccagtgaattgtaatacgactcactatagggaggcggatatggatccggcgcgccgtcgac